(19) United States
(12) Patent Application Publication
(10) Pub. No.: US 2026/0226493 A1
(43) Pub. Date: Aug. 6, 2026

(54) HERBICIDE RESISTANT PLANTS

(71) Applicant: SYNGENTA CROP PROTECTION AG, Basel (CH)

(72) Inventors: Richard Paul DALE, Bracknell, Berkshire (GB); Anthea Karin BATCHELOR, Bracknell, Berkshire (GB); David BROCKLEHURST, Bracknell, Berkshire (GB); Zane Lewis DUXBURY, Bracknell, Berkshire (GB); Emma Catherine LINNEY, Bracknell, Berkshire (GB)

(73) Assignee: SYNGENTA CROP PROTECTION AG, Basel (CH)

(21) Appl. No.: 19/153,098

(22) PCT Filed: Feb. 1, 2024
(86) PCT No.: PCT/EP2024/052561

§ 371 Date: Aug. 1, 2025

(30) Foreign Application Priority Data
Feb. 3, 2023 (EP) 23154964.3

(51) Int. Cl. C12N 15/82; C12N 9/00; C12N 9/10

(52) U.S. Cl. CPC C12N 15/8274; C12N 9/1096; C12N 9/93; C12N 15/8201; C07K 2319/07; C12Y 206/01062; C12Y 603/03003

(57) ABSTRACT

The present invention relates to plants and pans thereof that have been modified to comprise a BioA and/or BIO3-BIO1 (BioDA) enzyme which confers at least partial resistance to compounds which inhibit the biotin synthesis pathway, such as herbicides, wherein in some cases the enzymes may be overexpressed to confer resistance, or may comprise modifications which confer resistance. The invention further relates to such modified BioA or BIO3-BIO1 enzymes, having modifications which improve resistance to such compounds, as well as polynucleotides and proteins encoding such enzymes. The invention also relates to methods of growing and propagating such plants, improving plant growth and controlling unwanted vegetation using such plants and parts thereof.

### FIELD OF THE INVENTION

The invention relates to plants and parts thereof that have been modified to comprise a BioA or BIO3-BIO1 (BioDA) enzyme which confers at least partial resistance to compounds which inhibit the biotin synthesis pathway, such as herbicides. The invention further relates to modified BioA or BIO3-BIO1 enzymes, having modifications which improve resistance to such compounds, as well as polynucleotides and proteins encoding such enzymes. The invention also relates to methods of growing and propagating such plants, improving plant growth and controlling unwanted vegetation using such plants and parts thereof.

### BACKGROUND

The present invention relates to the production of plants that are resistant to herbicides that inhibit the biotin synthesis pathway, specifically to herbicides which inhibit the BIO3-BIO1 enzyme in plants.

Biotin, also known as vitamin B7, is an essential co-factor for enzymes involved in cellular processes including metabolism of fats, proteins or carbohydrates. Plants and most fungi/bacteria are able to synthesise biotin in contrast to animals which derive biotin from dietary sources or gut bacteria.

In bacteria, biotin is synthesised from pimeloyl-CoA and Alanine via the activity of four enzymes (BioF, BioA, BioD and BioB) which are located in an operon. BioF catalyses the production of 7-keto-8-Aminopelargonic Acid (KAPA) from pimeloyl-CoA and Alanine. BioA, also known as 7,8-diaminopelargonic acid aminotransferase (EC. 2.6.1.62) then carries out the next step, converting KAPA to 7,8 Diaminopelargonic Acid (DAPA). BioD, also known as dethiobiotin synthase (EC 6.3.3.3), subsequently converts DAPA to Dethiobiotin which is in turn converted to Biotin via the activity of BioB. (Entcheva et al, Applied Microbiology and Biotechnology volume 61, 21-31 (2003).

In plants, the pathway is similar, but the BioA and BioD enzyme activities are found in a single bifunctional protein known as BIO3-BIO1, BioDA, or bifunctional dethiobiotin synthetase. The BioD activity is found in the BIO3 sequence and BioA activity is provided by BIO1. Loss of function mutations in the BIO3-BIO1 gene of plants leads to an embryo lethal effect which can be rescued by exogenous application of biotin (Meinke et al, Plant Physiol. 2008 January; 146(1): 60-73.)

Given that the biotin pathway is essential to the survival of plants, it has been identified as a herbicidal target. Recently, effective herbicidal compounds which inhibit one or more of the enzymes of this pathway, especially BIO3-BIO1, have been developed. Upon contact with plants, the herbicides cause cell death and eventually death of the plant. Industrially, herbicides are used in agriculture to remove unwanted vegetation such as weeds from cultivated crops. However non-specific herbicides which target essential pathways such as those that target the biotin synthesis pathway, will affect both crops and the unwanted vegetation, making them difficult to use without destroying the valuable crop. Therefore in order to effectively use these herbicides, it would be desirable to protect the crop plants so that they have resistance to the herbicidal compounds. Plants that have resistance to the herbicides can then be contacted with the herbicide and will not be affected, whilst non-resistant unwanted vegetation is affected and controlled. Means to protect crop plants from other herbicides in the past have involved providing the plant with a mutated form of the enzyme which is targeted by the herbicide, thereby imparting resistance to the plant.

However, there has not yet been a link shown between modification of the BIO3-BIO1 genes or BioA gene, either by overexpression or mutagenesis, and increased resistance to herbicidal compounds which target the biotin pathway in plants.

### SUMMARY OF INVENTION

According to a first aspect of the present invention there is provided a plant, or part thereof, modified to comprise a BIO3-BIO1 and/or BioA enzyme that provides the plant or part thereof with increased resistance to a compound which inhibits the biotin synthesis pathway relative to an unmodified plant.
In one embodiment, there is provided a plant or a part thereof modified to comprise a polynucleotide encoding a BIO3-BIO1 and/or BioA enzyme, the expression of which provides the plant or part thereof with increased resistance to a compound which inhibits the biotin synthesis pathway relative to an unmodified plant.
In one embodiment, the plant or part thereof may be modified to comprise both a BIO3-BIO1 and a BioA enzyme which provide the plant or part thereof with increased resistance to a compound which inhibits the biotin synthesis pathway relative to an unmodified plant. Any of the aspects or embodiments herein may relate to a plant or part thereof comprising both enzymes, or modified to comprise both enzymes. However, in a preferred embodiment, the plant or part thereof is modified to comprise one of a BIO3-BIO1 enzyme or a BioA enzyme that provides the plant or part thereof with increased resistance to a compound which inhibits the biotin synthesis pathway relative to an unmodified plant.

According to a second aspect of the present invention there is provided a method of producing a modified plant or part thereof having an increased resistance to a compound which inhibits the biotin synthesis pathway relative to an unmodified plant, the method comprising: modifying the plant or part thereof to comprise a BIO3-BIO1 and/or BioA enzyme that provides the increased resistance.
In one embodiment, the method of producing a modified plant or part thereof having an Increased resistance to a compound which inhibits the biotin synthesis pathway relative to an unmodified plant, comprises: transforming the plant or part thereof with a polynucleotide encoding a BIO3-BIO1 and/or BioA enzyme, the expression of which provides the plant or part thereof with increased resistance to a compound which inhibits the biotin synthesis pathway relative to an unmodified plant.
In one embodiment, the polynucleotide is comprised on an expression construct or vector, and suitably comprises a plant promoter, the promoter being capable of driving expression of the polynucleotide.

According to a third aspect of the present invention there is provided a method for increasing the resistance of a plant or part thereof to a compound which inhibits the biotin synthesis pathway relative to an unmodified plant, the method comprising: modifying the plant or part thereof to comprise a BIO3-BIO1 and/or BioA enzyme that provides the increased resistance.
In one embodiment, the method of increasing the resistance of a plant or part thereof to a compound which inhibits the biotin synthesis pathway relative to an unmodified plant, comprises: transforming the plant or part thereof with a polynucleotide encoding a BIO3-BIO1 and/or BioA enzyme, the expression of which provides the plant or part thereof with increased resistance to a compound which inhibits the biotin synthesis pathway relative to an unmodified plant.
In one embodiment, the polynucleotide is comprised on an expression construct or vector, and suitably comprises a plant promoter, the promoter being capable of driving expression of the polynucleotide.

According to a third aspect of the present invention there is provided a modified plant or part thereof produced by the methods of the second or third aspects.
In one embodiment, the modified plant or part thereof comprises in at least some of its cells a BIO3-BIO1 and/or BioA enzyme that provides the plant or part thereof with increased resistance to a compound which inhibits the biotin synthesis pathway relative to an unmodified plant. Suitably comprising in at least some of its cells a polynucleotide encoding a BIO3-BIO1 and/or BioA enzyme, the expression of which provides the plant or part thereof with increased resistance to a compound which inhibits the biotin synthesis pathway relative to an unmodified plant.
In one embodiment, the modified plant part, such as a plant cell, is capable of regenerating a plant comprising in at least some of its cells a BIO3-BIO1 and/or BioA enzyme that provides the plant or part thereof with increased resistance to a compound which inhibits the biotin synthesis pathway relative to an unmodified plant. Suitably comprising in at least some of its cells a polynucleotide encoding a BIO3-BIO1 and/or BioA enzyme, the expression of which provides the plant or part thereof with increased resistance to a compound which inhibits the biotin synthesis pathway relative to an unmodified plant.
In one embodiment, the polynucleotide is comprised on an expression construct or vector, and suitably comprises a plant promoter, the promoter being capable of driving expression of the polynucleotide.

According to a fourth aspect of the present invention there is provided one or more seeds produced from the modified plant of the first or third aspects, or one or more plant products prepared from a modified plant of the first or third aspects.
In one embodiment, the seed is capable of germination into a plant comprising in at least some of its cells a BIO3-BIO1 and/or BioA enzyme that provides the plant or part thereof with increased resistance to a compound which inhibits the biotin synthesis pathway relative to an unmodified plant.
In one embodiment, the seed is capable of germination into a plant comprising in at least some of its cells a polynucleotide encoding a BIO3-BIO1 and/or BioA enzyme, the expression of which provides the plant or part thereof with increased resistance to a compound which inhibits the biotin synthesis pathway relative to an unmodified plant.
In one embodiment, the plant product prepared from the plant or part thereof comprises in at least some of its cells a BIO3-BIO1 and/or BioA enzyme which would provide a plant or part thereof with increased resistance to a compound which inhibits the biotin synthesis pathway relative to an unmodified plant.
In one embodiment, the plant product prepared from the plant or part thereof comprises in at least some of its cells a polynucleotide encoding a BIO3-BIO1 and/or BioA enzyme the expression of which would provide a plant or part thereof with increased resistance to a compound which inhibits the biotin synthesis pathway relative to an unmodified plant.
In one embodiment, the polynucleotide is comprised on an expression construct or vector, and suitably comprises a plant promoter, the promoter being capable of driving expression of the polynucleotide.

According to a fifth aspect of the present invention there is provided a modified BIO3-BIO1 enzyme having one or more of the following sequence motifs:

[Motif 1 (SEQ ID NO: 208)]
W; (H/Y/W); P; F; (A/Q/S/T); Q; (H/Q/V); X; X; X
[Motif 2 (SEQ ID NO: 209)]
(I/L/V); (D/E); (S/G); (R/A); X; (A/D/G/K); (E/D
/N); X; (F/Y)
[Motif 3 (SEQ ID NO: 210)]
(F/I/L/V/Y); D; (A/G); (C/I/P/S); (A/G/S); S; W;
W; (T/S/V); (I/Q)
[Motif 4 (SEQ ID NO: 211)]
(F/Y); (G/D); (H/Q); (A/I/V); (M/I/L); (F/L/Y);
(A/L/P); (E/G/H/L/N/S/T)
[Motif 5 (SEQ ID NO: 212)]
(A/G/T); (F/L/T/Q/V); X; (G/N/D/E/R); (S/C/G); Y
; H; G; D; T; (L/I/M); (G/S); (A/C/S/T/V); (M/L/
T); (D/E/N); X; (A/E/K/Q/R/S/T); (A/E/I/Q/V/T);
(E/G/I/K/P/S); (C/E/N/S/T); X; (F/T/Y); (M/N/S/
T); X
[Motif 6 (SEQ ID NO: 213)]
(A/E/K/Q/R/S/T); (E/H/I/Q/V/T); (D/E/H/P); (S/W);
(F/H/Y); X; (G/P/Q/R/S); (E/K/Q/R/W)
[Motif 7 (SEQ ID NO: 214)]
(F/S/W); (F/I/L/P); X; (A/C/F/P/V/Y); (P/S); X;
(I/L/M/T/V)
[Motif 8 (SEQ ID NO: 215)]
(A/G); (A/S); (A/G); G; M; X; (F/M/L); (A/C/I/V)
[Motif 9 (SEQ ID NO: 216)]
L; L; H; (G/R); H; S; (F/Y); (N/S/T); (A/G/T);
(H/N/Y)
[Motif 10 (SEQ ID NO: 217)]
(A/E/G/H/K/P/Q/S/V); A/C/G/K/N/Q/R/S/T/V/Y); (H/
I/L/V); (A/D/E/H/K/L/N/Q/R/S); (G/K/R/S/Y); (A/C
/V/L/T/V); (F/I/M/T/V/W); (A/D/G/N/S/V; (I/L/M/Q
/V)
[Motif 11 (SEQ ID NO: 218)]
(A/I/L/V/Y); (A/I/L/N/Q/R/V); (A/D/E/I/K/L/M/N/T
/Q/R/S); (A/D/E/F/H/K/M/N/Q/R/S/T/V/Y); (F/L);
(A/H/K/L/M/R/S/T/Y); X; X; (F/G)
[Motif 12 (SEQ ID NO: 219)]
(A/I/L/M/N/V); (F/H/L/Q/Y); (A/C/E/I/L/M/S/T); R;
(A/I/P/S/V); L; G; (D/K/N; I/T/V); (F/I/L/M/V);
Y

wherein one or more positions of the or each motif are mutated.
In one embodiment of the fifth aspect there is provided a modified BIO3-BIO1 enzyme having an amino acid sequence comprising one or more mutations at positions selected from: P347, F348, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, R797 of SEQ ID NO:1, or at corresponding positions thereto.
In one embodiment of the fifth aspect there is provided a modified BIO3-BIO1 enzyme having at least 30% identity to an amino acid sequence according to any of SEQ ID Numbers 1-14, 271-276 and 319 or a functional fragment thereof, wherein the amino acid sequence or fragment comprises one or more mutations at positions selected from: P347, F348, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, R797 defined in relation to SEQ ID NO:1, or at corresponding positions thereto, such as in SEQ ID NOs: 2 to 14, 271-276 and 319.

According to a sixth aspect of the present invention there is provided a modified BioA enzyme comprising an amino acid sequence having at least 30% identity to a sequence according to any of SEQ ID Numbers 159-199 or a functional fragment thereof. In some embodiments, the modified BioA enzyme of SEQ ID NO: 159-199 comprises a mitochondrial targeting peptide.
In one embodiment of the seventh aspect, there is provided a modified BioA enzyme comprising or consisting of an amino acid sequence according to SEQ ID Number 201.

According to a seventh aspect of the present invention there is provided an isolated polynucleotide encoding a modified BIO3-BIO1 enzyme according to the fifth aspect or a modified BioA enzyme according to the sixth aspect.

According to a eighth aspect of the present invention there is provided an expression construct comprising a polynucleotide encoding a modified BIO3-BIO1 enzyme according to the fifth aspect and/or a polynucleotide encoding a modified BioA enzyme according to the sixth aspect, operably linked to one or more expression elements.

According to a ninth aspect of the present invention there is provided a vector comprising the expression construct of the eighth aspect.

According to a tenth aspect of the present invention there is provided a plant or part thereof comprising one or more of: the modified BIO3-BIO1 enzyme according to the fifth aspect, the modified BioA enzyme according to the sixth aspect, the polynucleotide according to the seventh aspect, the expression construct according to the eighth aspect, the vector according to the ninth aspect.

According to an eleventh aspect of the present invention there is provided a method of controlling undesired vegetation in the vicinity of a plant or at the locus for growth of a plant according to the first aspect or twentieth aspect, the method comprising applying an effective amount of at least one compound which inhibits the biotin synthesis pathway to the undesired vegetation and the plant, or the locus, and optionally planting a seed at the locus wherein the seed is capable of producing a plant according to the first aspect or twentieth aspect.
In one embodiment, the seed is as defined according to the fourth aspect.

According to an twelfth aspect of the present invention there is provided a method of enhancing growth of a plant according to the first aspect or twentieth aspect, by controlling undesired vegetation in the vicinity of the plant, the method comprising applying an effective amount of at least one compound which inhibits the biotin synthesis pathway to the undesired vegetation and the plant.

According to a thirteenth aspect of the present invention there is provided the use of a compound which inhibits the biotin synthesis pathway in combination with a plant modified to comprise a BIO3-BIO1 and/or BioA enzyme that provides the plant or part thereof with increased resistance to said compound.
In one embodiment of the thirteenth aspect, the plant is as defined in the first aspect or twentieth aspect.

According to a fourteenth aspect of the present invention there is provided a kit comprising a container and instructions for use, the container comprising a compound which inhibits the biotin synthesis pathway, and the instructions comprising a direction to apply the compound to a plant modified to comprise a BIO3-BIO1 enzyme and/or BioA enzyme that provides the plant or part thereof with increased resistance to said compound.
In one embodiment of the fourteenth aspect, the plant is as defined in the first aspect or twentieth aspect.

According to a fifteenth aspect of the present invention there is provided the use of a plant according to the first aspect or twentieth aspect for breeding a plant variety or plant hybrid.

According to a sixteenth aspect of the present invention there is provided a method of producing a hybrid seed comprising crossing a first plant comprising the polynucleotide according to the seventh aspect, the expression construct according to the eighth aspect, or the vector according to the ninth aspect with a second plant; and obtaining one or more seeds therefrom.

According to a seventeenth aspect of the present invention there is provided the use of a modified BIO3-BIO1 enzyme according to the fifth aspect, or a polynucleotide encoding said enzyme according to the seventh aspect, as a selectable marker in plant transformation.
In one embodiment, the marker is used for selecting plants having an increased resistance to a compound which inhibits the biotin synthesis pathway relative to an unmodified plant.

According to an eighteenth aspect of the present invention, there is provided a method of selecting a plant comprising an increased resistance to a compound which inhibits the biotin synthesis pathway relative to an unmodified plant, comprising:
(a) Providing a plant;
(b) Optionally mutagenizing the plant;
(c) Exposing the plant to an effective amount of a compound which inhibits the biotin synthesis pathway; and
(d) Selecting the plant if the plant displays resistance to the compound.
In one embodiment the plant may have been produced by the method of the second aspect.

According to a nineteenth aspect of the present invention, there is provided a method of identifying a modified BIO3-BIO1 enzyme and/or a BioA enzyme which comprises an increased resistance to a compound which inhibits the biotin synthesis pathway, comprising:
(a) Generating a library of modified BIO3-BIO1 and/or BioA encoding polynucleotides;
(b) Screening a population of the resulting modified BIO3-BIO1 and/or BioA encoding polynucleotides by expressing each of said polynucleotides in a bacteria, a plant or a plant part and exposing the plant or part thereof to an effective amount of a compound which inhibits the biotin synthesis pathway;
(c) Selecting the modified BIO3-BIO1 and/or BioA encoding polynucleotides which provide the bacteria, plant or plant part thereof with increased resistance to said compound compared to an reference bacteria, plant or plant part thereof containing an unmodified BIO3-BIO1 and/or BioA encoding polynucleotide.

According to a twentieth aspect of the present invention, there is provided a method of identifying a compound which inhibits the biotin synthesis pathway comprising:
(a) generating a modified plant or part thereof of the first aspect or twentieth aspect;
(b) applying a test compound to the plant or part thereof of step (a) and to an unmodified reference plant;
(c) selecting the test compounds which confer reduced growth to the unmodified reference plant as compared to the growth of the modified plant or part thereof.

In one embodiment, the modified plant or part thereof is generated by a method according to the second or third aspects.

According to a twentieth aspect of the present invention, there is provided a plant, or part thereof, modified to comprise a heterologous BIO3-BIO1 enzyme that provides the plant or part thereof with increased resistance to a compound which inhibits the biotin synthesis pathway relative to an unmodified plant.
In one embodiment, the plant is as defined in the first aspect.
In one embodiment the plant may have been produced by the method of the second aspect.
In one embodiment, the heterologous BIO3-BIO1 enzyme is an Arabidopsis thaliana BIO3-BIO1 enzyme. For example, the heterologous BIO3-BIO1 enzyme comprises an amino acid sequence according to SEQ ID NOs: 1 or 294. For example, the heterologous BIO3-BIO1 enzyme consists of an amino acid sequence according to SEQ ID NOs: 1 or 294.
In one embodiment, the heterologous BIO3-BIO1 enzyme is an Zea mays BIO3-BIO1 enzyme. For example, the heterologous BIO3-BIO1 enzyme comprises an amino acid sequence according to SEQ ID NOs: 2 or 295. For example, the heterologous BIO3-BIO1 enzyme consists of an amino acid sequence according to SEQ ID NOs: 2 or 295.
In one embodiment, the heterologous BIO3-BIO1 enzyme is an Nannochloropsis gaditana BIO3-BIO1 enzyme. For example, the heterologous BIO3-BIO1 enzyme comprises an amino acid sequence according to SEQ ID NOs: 3 or 296. For example, the heterologous BIO3-BIO1 enzyme consists of an amino acid sequence according to SEQ ID NOs: 3 or 296.
In one embodiment, the heterologous BIO3-BIO1 enzyme is an Taxus chinensis BIO3-BIO1 enzyme. For example, the heterologous BIO3-BIO1 enzyme comprises an amino acid sequence according to SEQ ID NOs: 4 or 296. For example, the heterologous BIO3-BIO1 enzyme consists of an amino acid sequence according to SEQ ID NOs: 4 or 296.
In one embodiment, the heterologous BIO3-BIO1 enzyme is an Physcomitrium patens BIO3-BIO1 enzyme. For example, the heterologous BIO3-BIO1 enzyme comprises an amino acid sequence according to SEQ ID NOs: 5 or 297. For example, the heterologous BIO3-BIO1 enzyme consists of an amino acid sequence according to SEQ ID NOs: 5 or 297.
In one embodiment, the heterologous BIO3-BIO1 enzyme is an Adiantum nelumboides BIO3-BIO1 enzyme. For example, the heterologous BIO3-BIO1 enzyme comprises an amino acid sequence according to SEQ ID NOs: 6. For example, the heterologous BIO3-BIO1 enzyme consists of an amino acid sequence according to SEQ ID NOs: 6.
In one embodiment, the heterologous BIO3-BIO1 enzyme is an Setaria italica BIO3-BIO1 enzyme. For example, the heterologous BIO3-BIO1 enzyme comprises amino acid sequence according to SEQ ID NOs: 7 or 298. For example, the heterologous BIO3-BIO1 enzyme consists of an amino acid sequence according to SEQ ID NOs: 7 or 298.
In one embodiment, the heterologous BIO3-BIO1 enzyme is an Phoenix dactylifera BIO3-BIO1 enzyme. For example, the heterologous BIO3-BIO1 enzyme comprises an amino acid sequence according to SEQ ID NOs: 8 or 299. For example, the heterologous BIO3-BIO1 enzyme consists of an amino acid sequence according to SEQ ID NOs: 8 or 299.
In one embodiment, the heterologous BIO3-BIO1 enzyme is an Ostreococcus tauri BIO3-BIO1 enzyme. For example, the heterologous BIO3-BIO1 enzyme comprises an amino acid sequence according to SEQ ID NOs: 9 or 300. For example, the heterologous BIO3-BIO1 enzyme consists of an amino acid sequence according to SEQ ID NOs: 9 or 300.
In one embodiment, the heterologous BIO3-BIO1 enzyme is an Helianthus annuus BIO3-BIO1 enzyme. For example, the heterologous BIO3-BIO1 enzyme comprises an amino acid sequence according to SEQ ID NOs: 10 or 301. For example, the heterologous BIO3-BIO1 enzyme consists of an amino acid sequence according to SEQ ID NOs: 10 or 301.
In one embodiment, the heterologous BIO3-BIO1 enzyme is an Quercus robur BIO3-BIO1 enzyme. For example, the heterologous BIO3-BIO1 enzyme comprises an amino acid sequence according to SEQ ID NOs: 11 or 302. For example, the heterologous BIO3-BIO1 enzyme consists of an amino acid sequence according to SEQ ID NOs: 11 or 302.
In one embodiment, the heterologous BIO3-BIO1 enzyme is an Thraustotheca clavate BIO3-BIO1 enzyme. For example, the heterologous BIO3-BIO1 enzyme an amino acid sequence according to SEQ ID NOs: 12 or 303. For example, the heterologous BIO3-BIO1 enzyme consists of an amino acid sequence according to SEQ ID NOs: 12 or 303.
In one embodiment, the heterologous BIO3-BIO1 enzyme is an Schizosaccharomyces japonicus BIO3-BIO1 enzyme. For example, the heterologous BIO3-BIO1 enzyme comprises an amino acid sequence according to SEQ ID NOs: 13. For example, the heterologous BIO3-BIO1 enzyme consists of an amino acid sequence according to SEQ ID NOs: 13.
In one embodiment, the heterologous BIO3-BIO1 enzyme is an Gibberella zeae BIO3-BIO1 enzyme. For example, the heterologous BIO3-BIO1 enzyme comprises an amino acid sequence according to SEQ ID NOs: 14 or 304. For example, the heterologous BIO3-BIO1 enzyme consists of an amino acid sequence according to SEQ ID NOs: 14 or 304.
In one embodiment, the heterologous BIO3-BIO1 enzyme is an Hordeum vulgare BIO3-BIO1 enzyme. For example, the heterologous BIO3-BIO1 enzyme comprises an amino acid sequence according to SEQ ID NOs: 271 or 305. For example, the heterologous BIO3-BIO1 enzyme consists of an amino acid sequence according to SEQ ID NOs: 271 or 305.
In one embodiment, the heterologous BIO3-BIO1 enzyme is an Brassica napus BIO3-BIO1 enzyme. For example, the heterologous BIO3-BIO1 enzyme comprises an amino acid sequence according to SEQ ID NOs: 272 or 306. For example, the heterologous BIO3-BIO1 enzyme consists of an amino acid sequence according to SEQ ID NOs: 272 or 306.
In one embodiment, the heterologous BIO3-BIO1 enzyme is an Gossypium hirsutum BIO3-BIO1 enzyme. For example, the heterologous BIO3-BIO1 enzyme comprises an amino acid sequence according to SEQ ID NOs: 273 or 307. For example, the heterologous BIO3-BIO1 enzyme consists of an amino acid sequence according to SEQ ID NOs: 273 or 307.
In one embodiment, the heterologous BIO3-BIO1 enzyme is an Oryza sativa BIO3-BIO1 enzyme. For example, the heterologous BIO3-BIO1 enzyme comprises an amino acid sequence according to SEQ ID NOs: 274 or 308. For example, the heterologous BIO3-BIO1 enzyme consists of an amino acid sequence according to SEQ ID NOs: 274 or 308.
In one embodiment, the heterologous BIO3-BIO1 enzyme is an Glycine max BIO3-BIO1 enzyme. For example, the heterologous BIO3-BIO1 enzyme comprises an amino acid sequence according to SEQ ID NOs: 275 or 309. For example, the heterologous BIO3-BIO1 enzyme consists of an amino acid sequence according to SEQ ID NOs: 275 or 309.
In one embodiment, the heterologous BIO3-BIO1 enzyme is an Triticum aestivum BIO3-BIO1 enzyme. For example, the heterologous BIO3-BIO1 enzyme comprises an amino acid sequence according to SEQ ID NOs: 276 or 310. For example, the heterologous BIO3-BIO1 enzyme consists of an amino acid sequence according to SEQ ID NOs: 276 or 310.
In one embodiment, the heterologous BIO3-BIO1 enzyme is an Triticum aestivum BIO3-BIO1 enzyme. For example, the heterologous BIO3-BIO1 enzyme comprises an amino acid sequence according to SEQ ID NOs: 319 or 320. For example, the heterologous BIO3-BIO1 enzyme consists of an amino acid sequence according to SEQ ID NOs: 319 or 320.

The invention will now be further described under the following headed sections, features under any given section may apply to any embodiment or aspect in any combination.

### DETAILED DESCRIPTION

### Modified Plants

The invention primarily relates to plants that have been modified to comprise a BIO3-BIO1 and/or BioA enzyme which provides the plants with increased resistance to a compound which inhibits the biotin synthesis pathway. Suitable compounds which inhibit the biotin synthesis pathway are defined hereinbelow.

As used herein, reference to a “BIO3-BIO1 enzyme” and the amino acid sequences thereof also refers to and is intended to encompass isolated polynucleotides encoding such an enzyme.

BIO3-BIO1 refers to an enzyme that catalyses the conversion of 7-keto-8-Aminopelargonic Acid (KAPA) into Dethiobiotin, the final intermediate in the biotin pathway before the formation of biotin. BIO3-BIO1 is identified by the enzyme number EC 2.6.1.62. BIO3-BIO1 enzyme may refer to any protein that is capable of carrying out the conversion of KAPA into Dethiobiotin. Examples of suitable BIO3-BIO1 enzymes are provided herein in SEQ ID NOs 1 to 155. BIO3-BIO1 may also be referred to as ‘BioDA’ or bifunctional dethiobiotin synthetase, these terms are used interchangeably herein.

As used herein, reference to a “BioA enzyme” and the amino acid sequences thereof also refers to and is intended to encompass isolated polynucleotides encoding such an enzyme.

BioA refers to an enzyme that catalyzes the conversion of KAPA to 7,8 Diaminopelargonic Acid (DAPA) in the biotin pathway. BioA is identified by the enzyme number EC. 2.6.1.62. BioA enzyme may refer to any protein that is capable of carrying out the conversion of KAPA into DAPA. Examples of suitable BioA enzymes are provided herein in SEQ ID NOs 159 to 199. BioA may also be referred to as 7,8-diaminopelargonic acid (DAPA) aminotransferase, these terms are used interchangeably herein.

Suitably the plant has been modified to increase expression of a BIO3-BIO1 enzyme and/or a BioA enzyme, suitably within the plant or a part thereof. Suitably the plant may have been modified to overexpress a BIO3-BIO1 and/or BioA enzyme, suitably within the plant or a part thereof.

Suitably the expression of the BIO3-BIO1 and/or BioA enzyme is increased to a level which provides the plant with increased resistance to a compound that inhibits the biotin synthesis pathway relative to an unmodified plant.

Suitably the expression of the BIO3-BIO1 and/or BioA enzyme is increased to a level of at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% greater than the expression thereof in an unmodified plant.

Suitably the expression of the BIO3-BIO1 and/or BioA enzyme is increased to a level from 5% to 150%, from 10% to 150%, 20% to 150%, 30% to 150%, 40% to 150%, 50% to 150%, 60% to 150%, 70% to 150%, 80% to 150%, 90% to 150%, 100% to 150%, 110% to 150%, 120% to 150%, 130% to 150% or 140% to 150% greater than the expression thereof in an unmodified plant.

Suitably the expression of the BIO3-BIO1 and/or BioA enzyme is increased to a level from 5% to 100%, from 10% to 100%, 20% to 100%, 30% to 100%, 40% to 100%, 50% to 100%, 60% to 100%, 70% to 100%, 80% to 100%, 90% to 100% greater than the expression thereof in an unmodified plant.

Suitably the expression of the BIO3-BIO1 and/or BioA enzyme is increased to a level from 5% to 90%, from 10% to 90%, 20% to 90%, 30% to 90%, 40% to 90%, 50% to 90%, 60% to 90%, 70% to 90%, 80% to 90% greater than the expression thereof in an unmodified plant.

Suitably the expression of the BIO3-BIO1 and/or BioA enzyme is increased to a level from 5% to 80%, from 10% to 80%, 20% to 80%, 30% to 80%, 40% to 80%, 50% to 80%, 60% to 80%, 70% to 80%, greater than the expression thereof in an unmodified plant.

Suitably the expression of the BIO3-BIO1 and/or BioA enzyme is increased to a level from 5% to 70%, from 10% to 70%, 20% to 70%, 30% to 70%, 40% to 70%, 50% to 70%, 60% to 70% greater than the expression thereof in an unmodified plant.

Suitably the expression of the BIO3-BIO1 and/or BioA enzyme is increased to a level from 5% to 60%, from 10% to 60%, 20% to 60%, 30% to 60%, 40% to 60%, 50% to 60%, greater than the expression thereof in an unmodified plant.

Suitably the expression of the BIO3-BIO1 and/or BioA enzyme is increased to a level from 5% to 50%, from 10% to 50%, 20% to 50%, 30% to 50%, 40% to 50% greater than the expression thereof in an unmodified plant.

Suitably the expression of the BIO3-BIO1 and/or BioA enzyme is increased to a level from 5% to 40%, from 10% to 40%, 20% to 40%, 30% to 40%, greater than the expression thereof in an unmodified plant.

Suitably the expression of the BIO3-BIO1 and/or BioA enzyme is increased to a level from 5% to 30%, from 10% to 30%, 20% to 30%, greater than the expression thereof in an unmodified plant.

Suitably the expression of the BIO3-BIO1 and/or BioA enzyme is increased to a level from 5% to 20%, from 10% to 20% greater than the expression thereof in an unmodified plant.

Suitably the expression of the BIO3-BIO1 and/or BioA enzyme is increased to a level from 5% to 10% greater than the expression thereof in an unmodified plant.

Suitably increases in expression of an enzyme may be determined by measuring an increase in expression of the gene encoding the enzyme, such as by known molecular biology techniques including RT-PCR, qPCR, RNA-seq and the like. Alternatively, expression of the enzyme may be measured directly by other known molecular biology techniques including western blots, or fluorescence based imaging techniques.

Suitably the plant has been modified to increase activity of a BIO3-BIO1 enzyme and/or a BioA enzyme, suitably within the plant or a part thereof. Suitably the plant may have been modified to comprise a modified BIO3-BIO1 and/or BioA enzyme having increased activity when compared to the unmodified, suitably wildtype, BIO3-BIO1 and/or BioA, suitably within the plant or a part thereof.

Suitably the activity of the BIO3-BIO1 and/or BioA enzyme is increased to a level which provides the plant with increased resistance to a compound that inhibits the biotin synthesis pathway relative to an unmodified plant.

Suitably the activity of the BIO3-BIO1 and/or BioA enzyme is increased to a level of at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% greater than the expression thereof in an unmodified plant.

Suitably the activity of the BIO3-BIO1 and/or BioA enzyme is increased to a level from 5% to 150%, from 10% to 150%, 20% to 150%, 30% to 150%, 40% to 150%, 50% to 150%, 60% to 150%, 70% to 150%, 80% to 150%, 90% to 150%, 100% to 150%, 110% to 150%, 120% to 150%, 130% to 150% or 140% to 150% greater than the activity thereof in an unmodified plant.

Suitably the activity of the BIO3-BIO1 and/or BioA enzyme is increased to a level from 5% to 100%, from 10% to 100%, 20% to 100%, 30% to 100%, 40% to 100%, 50% to 100%, 60% to 100%, 70% to 100%, 80% to 100%, 90% to 100% greater than the activity thereof in an unmodified plant.

Suitably the activity of the BIO3-BIO1 and/or BioA enzyme is increased to a level from 5% to 90%, from 10% to 90%, 20% to 90%, 30% to 90%, 40% to 90%, 50% to 90%, 60% to 90%, 70% to 90%, 80% to 90% greater than the expression thereof in an activity plant.

Suitably the activity of the BIO3-BIO1 and/or BioA enzyme is increased to a level from 5% to 80%, from 10% to 80%, 20% to 80%, 30% to 80%, 40% to 80%, 50% to 80%, 60% to 80%, 70% to 80%, greater than the activity thereof in an unmodified plant.

Suitably the activity of the BIO3-BIO1 and/or BioA enzyme is increased to a level from 5% to 70%, from 10% to 70%, 20% to 70%, 30% to 70%, 40% to 70%, 50% to 70%, 60% to 70% greater than the activity thereof in an unmodified plant.

Suitably the activity of the BIO3-BIO1 and/or BioA enzyme is increased to a level from 5% to 60%, from 10% to 60%, 20% to 60%, 30% to 60%, 40% to 60%, 50% to 60%, greater than the activity thereof in an unmodified plant.

Suitably the activity of the BIO3-BIO1 and/or BioA enzyme is increased to a level from 5% to 50%, from 10% to 50%, 20% to 50%, 30% to 50%, 40% to 50% greater than the activity thereof in an unmodified plant.

Suitably the activity of the BIO3-BIO1 and/or BioA enzyme is increased to a level from 5% to 40%, from 10% to 40%, 20% to 40%, 30% to 40%, greater than the activity thereof in an unmodified plant.

Suitably the activity of the BIO3-BIO1 and/or BioA enzyme is increased to a level from 5% to 30%, from 10% to 30%, 20% to 30%, greater than the activity thereof in an unmodified plant.

Suitably the activity of the BIO3-BIO1 and/or BioA enzyme is increased to a level from 5% to 20%, from 10% to 20% greater than the activity thereof in an unmodified plant.

Suitably the activity of the BIO3-BIO1 and/or BioA enzyme is increased to a level from 5% to 10% greater than the activity thereof in an unmodified plant.

Suitably increase in the activity of an enzyme may be measured by an enzyme assay, which measures the consumption of a substrate or production of a product over time, suitably in an in vitro environment. Such assays may be spectrophotometric, fluorometric, calorimetric, chemiluminescent, light scattering or microscale thermophoresis. In one embodiment, a fluorometric assay as described in example 6 may be used, in which the fluorescent product produced by the reaction of DAPA with o-phthalaldehyde and β-mercaptoethanol is measured.

Suitably the plant may have been modified to increase the expression and/or the activity of a BIO3-BIO1 enzyme and/or a BioA enzyme, suitably within the plant or a part thereof. Suitably the increase in expression and the increase in activity are as defined above.

As noted above, the plant or part thereof may be modified to comprise either or both of a BIO3-BIO1 and a BioA enzyme which provide the plant or part thereof with increased resistance to a compound which inhibits the biotin synthesis pathway relative to an unmodified plant.

In some embodiments, the BIO3-BIO1 enzyme and/or the BioA enzyme may be a wild type enzyme, suitably an enzyme which is found in nature and which is unmodified. Suitably such wild type enzymes may be overexpressed in the plant, suitably to provide the plant with increased resistance to a compound that inhibits the biotin synthesis pathway relative to an unmodified plant.

The BIO3-BIO1 enzyme is found in plants, algae, fungi, or oomycetes. Therefore, suitably the BIO3-BIO1 enzyme may be an endogenous or a heterologous enzyme to the plant. Suitably the BIO3-BIO1 enzyme may be derived from a plant, from an algae, or a from a fungus. In one embodiment, the BIO13-BIO1 enzyme is derived from a plant. In one embodiment, the BIO3-BIO1 enzyme is introduced into a heterologous plant species. In another embodiment, the BIO3-BIO1 enzyme is introduced into a plant of the same species or to a crossable plant species. In one embodiment, the BIO13-BIO1 enzyme is derived from an algae. In one embodiment, the BIO13-BIO1 enzyme is derived from a fungus.

Suitably a BIO3-BIO1 or BioA enzyme may be defined by comprising a common motif, which is suitably shared by most BIO3-BIO1 and by most BioA enzymes. Suitably the BIO3-BIO1 or BioA enzyme comprises any one or more of the following motifs:

[Motif 13: SEQ ID NO: 220]
(A/C/G/S); (F/Y); H; G; (D/E); T; (F/I/L/M/V/W);
(A/D/E/G/K/M/Q); (A/C/G/P/T/V); (I/L/M/V); (A/D
/E/N/S); (A/C/I/L/M/T/V)
[Motif 14: SEQ ID NO: 221]
(F/Y); (F/L/Y); (A/C/N/S/V); D; (D/N/S); G; (A/S
); (A/C/E/I/S/T/V); (A/C/G/S); (C/I/M/T/V); (D/E
); (C/I/V); (A/G/S); (I/L/M/V); K; (A/I/M/V); (A
/C/S/V)

Alternatively, or additionally, a BIO3-BIO1 or BioA enzyme may be defined by comprising a common motif, which is suitably shared by most BIO3-BIO1 and by most BioA enzymes, with the exception of BioA from Escherichia coli. In such an embodiment, the BIO3-BIO1 or BioA enzyme comprises the following motif:

(SEQ ID NO: 270)
(H/Q); (C/I/M/S/T/V); (I/L/M/T/V); (F/L/Y);
(A/G/L/P); (D/E/G/M/N); (A/C/F/I/L/M/N/Y);
(A/G/I/L/S/T/V); (H/N/Y); (E/K/N/P/Q/S/V);
(A/G/K/Q/P/T)(Motif 17)

Wherein the amino acid residues are given their standard single letter code, wherein alternate amino acids at a given position are indicated in parentheses, and wherein ‘X’ indicates any amino acid.

Suitably the BIO3-BIO1 or BioA enzyme may comprise any combination of the above motifs 13, 14, and/or 17. Suitably the BIO3-BIO1 or BioA enzyme may comprise one or all of the above motifs 13, 14, and 17. Suitably therefore, a BIO3-BIO1 or BioA enzyme may be defined by comprising one or more of the above motifs 13, 14 and 17.

Suitably the BIO3-BIO1 or BioA enzyme may comprise an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence of SEQ ID NO: 1 to 14, 271-276 and 319 (or SEQ ID NO: 294 to 310 and 320), or 159 to 199 respectively, or a functional fragment thereof. Suitably the BIO3-BIO1 or BioA enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 1 to 14, 271-276 and 319, (or SEQ ID NO: 294 to 310 and 320), or 159 to 199 respectively, or a functional fragment thereof, and comprises one or more of the above motifs 13, 14, and/or 17. Suitably the BIO3-BIO1 or BioA enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 1 to 14, 271-276 and 319, (or SEQ ID NO: 294 to 310 and 320), or 159 to 199 respectively, or a functional fragment thereof, and comprises the above motifs 13, 14 and 17.

Suitably herein reference may be made to the BIO3-BIO1 enzymes according to SEQ ID NOs 1 to 14 and 271 to 276, such references may equally be replaced throughout the present disclosure with references to the BIO3-BIO1 enzymes according to SEQ ID NOs 294 to 310 and 320. Suitably the BIO3-BIO1 enzymes according to SEQ ID NOs 1-4 and 271-276 and 319 are the same as the sequences according to SEQ ID NOs 294 to 310 and 320 with the exception that SEQ ID NOs 294 to 310 and 320 do not comprise a targeting peptide. Suitably any reference herein to SEQ ID NOs 1-14, 271 to 276, may be replaced with the corresponding sequence from the same organism as defined in SEQ ID NOs 294 to 310 and 320, for example SEQ ID NO:1 may be replaced with SEQ ID NO:294, SEQ ID NO:2 may be replaced with SEQ ID NO:295, etc.

Suitably therefore BIO3-BIO1 enzyme may comprise an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence of SEQ ID NO: 294 to 310 and 320, or a functional fragment thereof. Suitably the BIO3-BIO1 enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 294 to 310 and 320, or a functional fragment thereof, and comprises one or more of the above motifs 13, 14 and/or 17. Suitably the BIO3-BIO1 enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 294 to 310 and 320, or a functional fragment thereof, and comprises the above motifs 13, 14 and 17.

Suitably a BIO3-BIO1 enzyme may be defined by comprising an amino acid motif, which is suitably shared by most BIO3-BIO1 enzymes. Suitably the BIO3-BIO1 enzyme comprises any one or more of the following motifs:

[Motif 1 (SEQ ID NO: 208)]
W; (H/Y/W); P; F; (A/Q/S/T); Q; (H/Q/V);
X; X; X
[Motif 2 (SEQ ID NO: 209)]
(I/L/V); (D/E); (S/G); (R/A); X; (A/D/G/K);
(E/D/N); X; (F/Y)
[Motif 3 (SEQ ID NO: 210)]
(F/I/L/V/Y); D; (A/G); (C/I/P/S); (A/G/S);
S; W; W; (T/S/V); (I/Q)
[Motif 4 (SEQ ID NO: 211)]
(F/Y); (G/D); (H/Q); (A/I/V); (M/I/L); (F/L/Y);
(A/L/P); (E/G/H/L/N/S/T)
[Motif 5 (SEQ ID NO: 212)]
(A/G/T); (F/L/T/Q/V); X; (G/N/D/E/R); (S/C/G);
Y; H; G; D; T; (L/I/M); (G/S); (A/C/S/T/V); (M/
L/T); (D/E/N); X; (A/E/K/Q/R/S/T); (A/E/I/Q/V/T);
(E/G/I/K/P/S); (C/E/N/S/T); X; (F/T/Y); (M/N/S/T);
X
[Motif 6 (SEQ ID NO: 213)]
(A/E/K/Q/R/S/T); (E/H/I/Q/V/T); (D/E/H/P); (S/W);
(F/H/Y); X; (G/P/Q/R/S); (E/K/Q/R/W)
[Motif 7 (SEQ ID NO: 214)]
(F/S/W); (F/I/L/P); X; (A/C/F/P/V/Y); (P/S); X;
(I/L/M/T/V)
[Motif 8 (SEQ ID NO: 215)]
(A/G); (A/S); (A/G); G; M; X; (F/M/L); (A/C/I/V)
[Motif 9 (SEQ ID NO: 216)]
L; L; H; (G/R); H; S; (F/Y); (N/S/T); (A/G/T);
(H/N/Y)
[Motif 10 (SEQ ID NO: 217)]
(A/E/G/H/K/P/Q/S/V); A/C/G/K/N/Q/R/S/T/V/Y);
(H/I/L/V);
(A/D/E/H/K/L/N/Q/R/S); (G/K/R/S/Y); (A/C/I/L/T/V);
(F/I/M/T/V/W); (A/D/G/N/S/V; (I/L/M/Q/V)
[Motif 11 (SEQ ID NO: 218)]
(A/I/L/V/Y); (A/I/L/N/Q/R/V); (A/D/E/I/K/L/M/N/
T/Q/R/S);(A/D/E/F/H/K/M/N/Q/R/S/T/V/Y); (F/L);
(A/H/K/L/M/R/S/T/Y); X; X; (F/G)
[Motif 12 (SEQ ID NO: 219)]
(A/I/L/M/N/V); (F/H/L/Q/Y); (A/C/E/I/L/M/S/T); R;
(A/I/P/S/V); L; G; (D/K/N; I/T/V); (F/I/L/M/V); Y

Wherein the amino acid residues are given their standard single letter code, wherein alternate amino acids at a given position are indicated in parentheses, and wherein ‘X’ indicates any amino acid.

Suitably the BIO3-BIO1 enzyme may comprise any combination of the above motifs 1 to 12. Suitably the BIO3-BIO1 enzyme may comprise any of motif 1, 2, 3, 4, 5, 6, 7, 8, 9, 10, 11 12, 13, 14 and/or 17. Suitably the BIO3-BIO1 enzyme may comprise all of the above motifs 1 to 14 and 17.

Suitably the BIO3-BIO1 enzyme may be derived from any plant species. Suitably the BIO3-BIO1 enzyme may be derived from any of the following plant species: Arabidopsis thaliana, Zea mays, Quercus robur, Triticum aestivum, Glycine max, Setaria italica, Oryza sativa, Heliosperma pusillum, Taxus chinensis, Carpinus fangiana, Cinnamomum micranthum, Apostasia shenzhenica, Asparagus officinalis, Phoenix dactylifera, Zostera marina, Amborella trichopoda, Adiantum nelumboides Echinochloa crus-galli, Zingiber officinale, Thlaspi arvense, Vitis vinifera, Helianthus annuus, Brassica oleracea, Hordeum vulgare, Brassica napus, Selaginella moellendorffii, Gossypium hirsutum and Amaranthus palmeri. In one embodiment, the BIO3-BIO1 enzyme may be derived from any of the following plant species: Setaria italica, Arabidopsis thaliana, Helianthus annuus, Quercus robur, Phoenix dactylifera, Physcomitrium patens, Taxus chinensis, Adiantum nelumboides, Zea mays, Hordeum vulgare, Brassica napus, Gossypium hirsutum, Oryza sativa, Triticum aestivum, Selaginella moellendorffii and Glycine max. In one embodiment, the BIO3-BIO1 enzyme is derived from Arabidopsis thaliana, or Zea mays. In other embodiments, the BIO3-BIO1 enzyme is the endogenous BIO3-BIO1 enzyme from a plant of interest.

Suitably the BIO3-BIO1 enzyme may be derived from any algal species. Suitably the BIO3-BIO1 enzyme may be derived from any of the following species of algae: Nannochloropsis gaditana, Pedinophyceae sp., Trebouxia sp., Ostreococcus tauri, and Micromonas pusilla. In one embodiment, the BIO3-BIO1 enzyme may be derived from any of the following species of algae: Nannochloropsis gaditana, and Ostreococcus tauri. In one embodiment, the BIO3-BIO1 enzyme is derived from Nannochloropsis gaditana.

Suitably the BIO3-BIO1 enzyme may be derived from any fungal species. Suitably the BIO3-BIO1 enzyme may be derived from any of the following species of fungi: Aspergillus candidus, Blastocladiella emersonii, Paraphysoderma sedebokerense, Talaromyces proteolyticus, Pseudomassariella vexata, Microthyrium microscopicum, Lophium mytilinum, Monilinia fructicola, Cryomyces minteri, Coniosporium apollinis, Polytolypa hystricis, Xylona heveae, Calocera cornea, Rhinocladiella mackenziei, Coniosporium apollinis, Schizosaccharomyces japonicus, Yarrowia lipolytica, Aspergillus niger, Gibberella zeae, and Aspergillus nidulans. In one embodiment, the BIO3-BIO1 enzyme may be derived from any of the following species of fungi: Schizosaccharomyces japonicus and Gibberella zeae.

Suitably the BIO3-BIO1 enzyme may be derived from any oomycete species. Suitably the BIO3-BIO1 enzyme may be derived from any of the following species of oomycete: Thraustotheca clavata, Albugo laibachii, Achlya hypogyna, Phytophthora spp. such as Phytophthora cactorum, Phytophthora rubi, Phytophthora capsici, and Phytophthora sojae. In one embodiment the BIO3-BIO1 enzyme may be derived from any of the following species of oomycete: Thraustotheca clavata,

Suitably the BIO3-BIO1 enzyme comprises an amino acid sequence having at least 30% identity to an amino acid sequence of SEQ ID NO: 1 to 14, 271-276 and 319, (or SEQ ID NO: 294 to 310 and 320), or a functional fragment thereof. Suitably the BIO3-BIO1 enzyme comprises an amino acid sequence having at least 30% identity to an amino acid sequence of SEQ ID NO: 1 to 14, 271-276 and 319, (or SEQ ID NO: 294 to 310 and 320), or a functional fragment thereof, and comprises one or more of motifs 1 to 14 and/or 17. Suitably the BIO3-BIO1 enzyme comprises an amino acid sequence having at least 30% identity to an amino acid sequence of SEQ ID NO: 1 to 14, 271-276 and 319, (or SEQ ID NO: 294 to 310 and 320), or a functional fragment thereof, and comprises motifs 1 to 14 or any combination of one or more motifs 1, 2, 3, 4, 5, 6, 7, 8, 9, 10, 11, 12, 13, 14, or 17.

Suitably the BIO3-BIO1 enzyme may comprise an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity to an amino acid sequence of SEQ ID NO: 1 to 14, 271-276 and 319, (or SEQ ID NO: 294 to 310 and 320), or a functional fragment thereof. Suitably the BIO3-BIO1 enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 1 to 14, 271-276 and 319, (or SEQ ID NO: 294 to 310 and 320), or a functional fragment thereof, and comprises one or more of motifs 1 to 14, and/or 17. Suitably the BIO3-BIO1 enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 1 to 14, 271-276 and 319, (or SEQ ID NO: 294 to 310 and 320), or a functional fragment thereof, and comprises motifs 1 to 14 and 17.

In some embodiments, the BIO3-BIO1 enzyme may consist of an amino acid sequence according to SEQ ID NO: 1 to 14, 271-276 and 319, (or SEQ ID NO: 294 to 310 and 320), or a functional fragment thereof.

In one embodiment, the BIO3-BIO1 enzyme is derived from Arabidopsis thaliana and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO:1. In one embodiment, the BIO3-BIO1 enzyme is derived from Arabidopsis thaliana and consists of an amino acid sequence according to SEQ ID NO:1.

In one embodiment, the BIO3-BIO1 enzyme is derived from Zea mays and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO:2. In one embodiment, the BIO3-BIO1 enzyme is derived from Zea mays and consists of an amino acid sequence according to SEQ ID NO:2.

In one embodiment, the BIO3-BIO1 enzyme is derived from Nannochloropsis gaditana and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO:3. In one embodiment, the BIO3-BIO1 enzyme is derived from Nannochloropsis gaditana and consists of an amino acid sequence according to SEQ ID NO:3.

In one embodiment, the BIO3-BIO1 enzyme is derived from Ostreococcus tauri and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO:9. In one embodiment, the BIO3-BIO1 enzyme is derived from Ostreococcus tauri and consists of an amino acid sequence according to SEQ ID NO:9.

In one embodiment, the BIO3-BIO1 enzyme is derived from Schizosaccharomyces japonicus and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO:13. In one embodiment, the BIO3-BIO1 enzyme is derived from Schizosaccharomyces japonicus and consists of an amino acid sequence according to SEQ ID NO:13.

In one embodiment, the BIO3-BIO1 enzyme is derived from Selaginella moellendorffii and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO:319. In one embodiment, the BIO3-BIO1 enzyme is derived from Selaginella moellendorffii and consists of an amino acid sequence according to SEQ ID NO:319.

In one embodiment, the BIO3-BIO1 enzyme is derived from Oryza sativa and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO:274. In one embodiment, the BIO3-BIO1 enzyme is derived from Oryza sativa and consists of an amino acid sequence according to SEQ ID NO:274.

In one embodiment, the BIO3-BIO1 enzyme is derived from Helianthus annuus and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO:10. In one embodiment, the BIO3-BIO1 enzyme is derived from Helianthus annuus and consists of an amino acid sequence according to SEQ ID NO:10.

In one embodiment, the BIO3-BIO1 enzyme is derived from Setaria italica and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO:7. In one embodiment, the BIO3-BIO1 enzyme is derived from Setaria italica and consists of an amino acid sequence according to SEQ ID NO:7.

The BioA enzyme is a found in bacteria. Therefore suitably the BioA enzyme is always heterologous to the plant. In one embodiment, the BioA enzyme is derived from a bacterium.

Suitably a BioA enzyme may be defined by comprising an amino acid motif, which is suitably shared by most BioA enzymes. Suitably a BioA enzyme comprises any one or more of the following motifs:

Motif 15
(SEQ ID NO: 268)
(A/G/S); (F/Y); H; G; (D/E); T; (F/I/L/M/V/W);
(A/D/E/G/K/M/Q); (A/G/P/T); (I/L/M/V); (A/E/S);
(A/I/L/T/V)
Motif 16
(SEQ ID NO: 269)
D; E; (I/V); (A/F/L/M); (T/V); G; (F/L/W); G;
(K/R); (C/S/T); G; (A/E/K/L/Q/P/R/S/T); (F/L
/M/N/R/W); F; (A/G/S)

Wherein the amino acid residues are given their standard single letter code, wherein alternate amino acids at a given position are indicated in parentheses, and wherein ‘X’ indicates any amino acid.

Suitably a BioA enzyme may comprise any combination of the above motifs 13, 14, 15, 16 and/or 17. Suitably the BioA enzyme may comprise all of the above motifs 13, 14, 15,16 and 17. Suitably a BioA enzyme may comprise any combination of the above motifs 15 and/or 16.

Suitably the BioA enzyme may be derived from any bacterial, protist, or archaeon species. Suitably the BioA enzyme is derived from any of the following bacterial, protist, or archaeon species: E. coli, Cryptosporidium andersoni, Agrobacterium tumefaciens, Citrobacter portucalensis, Cedecea sp. nfix57 BioA, Xenorhabdus sp. xeno-1, Methanobrevibacter olleyae, Bacillus subtilis, Pantoea ananatis, Bacillus licheniformis, Streptomyces hygroscopicus, Streptomyces viridochromogenes, Stenotrophomonas maltophilia, Pseudomonas fluorescens, Bacillus thuringiensis, Candidatus Midichloria mitochondrii, Thiofilum flexile, Panacibacter microcysteis, Spirosoma linguale, Prochlorococcus marinus, Chitinophaga polysaccharea, Chitinophaga filiformis, Pedobacterhartonius, Fluviicola spxm-24bin1, Flavobacterium foetidum, Chroococcidiopsis sp. ccmee 29, Tenacibaculum adriaticum, Pseudobacteriovorax antillogorgiicola, Texcoconibacillus texcoconensis, Nitrobacter sp. 62-13, Wigglesworthia glossinidia, Methylomarinum vadi, Flocculibacter collagenilyticus, Leptolyngbya ectocarpi, Psychrosphaera aestuarii, Fragilariopsis cylindrus, Deferrisoma camini, Chlorobaculum tepidum, Chlamydia pneumoniae, Pedobacter psychrophilus, and Pseudopedobacter saltans.

Suitably the BioA enzyme may be derived from Bacillus subtilis. Suitably the BioA enzyme may be derived from Pantoea ananatis. Suitably the BioA enzyme may be derived from Stenotrophomonas maltophilia. Suitably the BioA enzyme may be derived from Chroococcidiopsis sp. CCMEE 29. Suitably the BioA enzyme may be derived from Streptomyces viridochromogenes. Suitably the BioA enzyme may be derived from Pedobacter hartonius.

Suitably the BioA enzyme may be derived fromChitinophaga filiformis. Suitably the BioA enzyme may be derived from Pedobacter hartonius. Suitably the BioA enzyme may be derived from Tenacibaculum adriaticum. Suitably the BioA enzyme may be derived from Streptomyces hygroscopicus.

Suitably the BioA enzyme comprises an amino acid sequence having at least 30% identity to an amino acid sequence of SEQ ID NO: 159 to 199 or a functional fragment thereof. Suitably the BioA enzyme comprises an amino acid sequence having at least 30% identity to an amino acid sequence of SEQ ID NO: 159 to 199 or a functional fragment thereof, and comprises one or more of motifs 13, 14, 15, 16 and/or 17. Suitably the BioA enzyme comprises an amino acid sequence having at least 30% identity to an amino acid sequence of SEQ ID NO: 159 to 199, or a functional fragment thereof, and comprises motifs 13, 14, 15, 16 and/or 17.

Suitably the BioA enzyme may comprise an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence of SEQ ID NO: 159 to 199 or a functional fragment thereof. Suitably the BioA enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 159 to 199 or a functional fragment thereof, and comprises one or more of motifs 13, 14, 15, 16 and/or 17. Suitably the BioA enzyme may comprise an amino acid sequence having at least 80%, at least 90%, at least 91%, at least 92%, at least 93%, at least 94%, at least 95%, at least 96%, at least 97%, at least 98%, at least 99% identity to an amino acid sequence of SEQ ID NO: 159 to 199 or a functional fragment thereof, and comprises motifs 13, 14, 15, 16 and 17.

In some embodiments the BioA enzyme may consist of an amino acid sequence according to SEQ ID NO: 159 to 199 or a functional fragment thereof. In one embodiment, the BioA enzyme is derived from E. coli and consists of an amino acid sequence according to SEQ ID NO:159.

In one embodiment, the BioA enzyme is derived from Pantoea ananatis and comprises an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence according to SEQ ID NO:167. In one embodiment, the BioA enzyme is derived from Pantoea ananatis and consists of an amino acid sequence according to SEQ ID NO:167.

In one embodiment, the BioA enzyme is derived from Stenotrophomonas maltophilia and comprises an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence according to SEQ ID NO:171. In one embodiment, the BioA enzyme is derived from Stenotrophomonas maltophilia and consists of an amino acid sequence according to SEQ ID NO:171.

In one embodiment, the BioA enzyme is derived from Chroococcidiopsis sp. CCMEE 29 and comprises an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence according to SEQ ID NO:184. In one embodiment, the BioA enzyme is derived from Chroococcidiopsis sp. CCMEE 29 and consists of an amino acid sequence according to SEQ ID NO:184.

In one embodiment, the BioA enzyme is derived from Bacillus subtilis and comprises an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence according to SEQ ID NO:166. In one embodiment, the BioA enzyme is derived from Bacillus subtilis and consists of an amino acid sequence according to SEQ ID NO:166.

In one embodiment, the BioA enzyme is derived from Streptomyces viridochromogenes and comprises an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence according to SEQ ID NO:170. In one embodiment, the BioA enzyme is derived from Streptomyces viridochromogenes and consists of an amino acid sequence according to SEQ ID NO:170.

In one embodiment, the BioA enzyme is derived from Pedobacter hartonius and comprises an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence according to SEQ ID NO:181. In one embodiment, the BioA enzyme is derived from Pedobacter hartonius and consists of an amino acid sequence according to SEQ ID NO:181.

In one embodiment, the BioA enzyme is derived from Chitinophaga filiformis and comprises an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence according to SEQ ID NO: 180. In one embodiment, the BioA enzyme is derived from Chitinophaga filiformis and consists of an amino acid sequence according to SEQ ID NO:180.

In one embodiment, the BioA enzyme is derived from Tenacibaculum adriaticum and comprises an amino acid sequence having at least 35%, at least 40%, at least 45%, at least 50%, at least 55%, at least 60%, at least 65%, at least 70%, at least 75%, least 80%, at least 90%, at least 91%, at least 92%, at least 93%, at least 94%, at least 95%, at least 96%, at least 97%, at least 98%, at least 99% identity to an amino acid sequence according to SEQ ID NO:185. In one embodiment, the BioA enzyme is derived from Tenacibaculum adriaticum and consists of an amino acid sequence according to SEQ ID NO:185.

Alternatively, instead of using wild-type BIO3-BIO1 or BioA enzymes in the present invention, the BIO3-BIO1 and/or BioA enzymes may be modified. In some embodiments, the BIO3-BIO1 and/or BioA enzymes may comprise one or more modifications, suitably one or more mutations. Suitably in such embodiments, the plant has been modified to comprise a BIO3-BIO1 and/or BioA enzyme having one or more mutations.

In one embodiment, the plant or part thereof may be modified to comprise both a BIO3-BIO1 and a BioA enzyme wherein one or both of the enzymes comprises one or more mutations which provide the plant or part thereof with increased resistance to a compound which inhibits the biotin synthesis pathway relative to an unmodified plant.

Suitably the one or more mutations provide the enzyme, and therefore the plant, with increased resistance to a compound which inhibits the biotin synthesis pathway relative to an unmodified plant.

Suitably in some embodiments, the BIO3-BIO1 and/or BioA enzyme may be modified and may also be overexpressed in the plant or part thereof. Suitable increases in expression and overexpression are described above. Suitable modifications are described below. Suitably the one or more modifications and the increased expression provide the plant with increased resistance to a compound which inhibits the biotin synthesis pathway relative to an unmodified plant.

Suitably the one or more mutations are selection from deletions, insertions, substitutions etc. In one embodiment the mutations are amino acid substitutions. Suitable modifications to the BIO3-BIO1 and/or BioA enzymes are defined in the relevant sections hereinbelow. As explained therein, in one embodiment, the BIO3-BIO1 enzyme is modified, in one embodiment the BIO3-BIO1 enzyme comprises one or more amino acid substitutions. In another embodiment, the native mitochondrial targeting peptide of the wild type BIO3-BIO1 enzyme or the modified BIO3-BIO1 enzyme is replaced with a heterologous mitochondrial targeting peptide, optionally in addition to the one or more amino acid substitutions. In one embodiment, the BioA enzyme is not modified and is a wild type enzyme fused to a mitochondrial targeting peptide. In another embodiment, a modified BioA enzyme is fused to a heterologous mitochondrial targeting peptide.

The plants of the present invention include both non-transgenic plants and transgenic plants.

By “non-transgenic plant” is intended to mean a plant lacking recombinant DNA in its genome, but containing a mutant nucleic acid molecule in the plant cell genome which has been mutated using mutagenic techniques, such as chemical mutagenesis, gene editing or by those methods provided herein. Non-transgenic plants may encompass those plants having mutant or modified sequences as a result of natural processes, such as plants including spontaneous BIO3-BIO1 enzymes that provide the desired resistance to compounds that inhibit the biotin synthesis pathway or by the use of gene editing techniques. In specific embodiments, the non-transgenic plant comprises a modified BIO3-BIO1 enzyme that has been altered through gene editing to comprise at least one or more of the modifications disclosed herein. Such gene editing modifications will increase the resistance of the plant to the herbicide of interest.

By “transgenic plant” is intended to mean a plant comprising recombinant DNA in its genome. As used herein, “recombinant,” when referring to nucleic acid or polypeptide, indicates that such material has been altered as a result of human application of a recombinant technique, such as by polynucleotide restriction and ligation, by polynucleotide overlap-extension, or by genomic insertion or transformation. In one embodiment, recombinant in relation to nucleic acids or polypeptides refers to nucleic acids or polypeptides that are produced or altered outside of a host cell into which they are intended to be transformed (such as a plant cell or plant as described herein). Therefore ‘transgenic plants’ as referred to herein are not produced by gene editing. A gene sequence open reading frame is recombinant if that nucleotide sequence has been removed from it natural text and cloned into any type of artificial nucleic acid vector. The term recombinant also can refer to an organism having a recombinant material, e.g., a plant that comprises a recombinant nucleic acid can be considered a recombinant plant. Such a transgenic plant can be produced by introducing recombinant DNA into the genome of the plant. When such recombinant DNA is incorporated into the genome of the transgenic plant, progeny of the plant can also comprise the recombinant DNA. A progeny plant that comprises at least a portion of the recombinant DNA of at least one progenitor transgenic plant is also a transgenic plant.

As used herein, “heterologous” in reference to a polypeptide or polynucleotide sequence is a sequence that originates, for example, from a cell or an organism from a foreign species. Alternatively, if the sequence originates from the same species, it is derived from a cell or organism having a different genetic background; or if from the same genetic background, it is substantially modified from its native form in composition and/or genomic locus by deliberate human intervention. As such, heterologous sequences are in a configuration not found in nature.

The term “spontaneous mutant” refers to mutants or variants that arise from the parent strain without the intentional use of mutagens i.e. they are considered as not genetically modified (non-GMO). Spontaneous mutants in respect of plants may also be known as sports, breaks, or chimeras.

Suitably the plant or part thereof of the invention is transgenic. In other embodiments, the plant or plant part thereof of the invention is non-transgenic and comprises a gene edit that increases the plant's or plant part's tolerance to a herbicide of interest.

In one embodiment, therefore, the plant or part thereof comprises a recombinant polynucleotide encoding a BIO3-BIO1 and/or BioA enzyme. Suitably the recombinant polynucleotide may be operable to express the BIO3-BIO1 and/or BioA enzyme at increased levels compared to an unmodified plant. Suitably wherein the increased expression of said polynucleotide provides or confers to the plant or part thereof an increased resistance to a compound which inhibits the biotin synthesis pathway as defined above. In such an embodiment, suitably the BIO3-BIO1 and/or BioA enzyme may be a wild type enzyme as described hereinabove.

In another embodiment, the plant or part thereof comprises a polynucleotide encoding a modified or mutated BIO3-BIO1 and/or BioA enzyme. Suitably the polynucleotide encoding the BIO3-BIO1 and/or BioA enzyme may comprise one or more modifications. Suitably therefore the polynucleotide may be operable to express a BIO3-BIO1 and/or BioA enzyme having one or more modifications or mutations. Suitably wherein the expression of said polynucleotide provides or confers to the plant or part thereof increased resistance to a compound which inhibits the biotin synthesis pathway. Suitably the or each modification in the BIO3-BIO1 and/or BioA enzyme provides increased resistance to a compound which inhibits the biotin synthesis pathway. Suitable such modifications are defined hereinbelow. In specific embodiments, the modified or mutated BIO3-BIO1 and/or BioA enyzme is encoded by a recombinant polynucleotide stably integrated into the plant's genome. Alternatively, the modified or mutated BIO3-BIO1 and/or BioA enzyme is encoded by a polynucleotide, suitably a gene, comprising a non-transgenic modification, such as an edit, within the genome of the plant. Suitably in such embodiments, the polynucleotide encoding a modified or mutated BIO3-BIO1 and/or BioA enzyme may also be operable to express the BIO3-BIO1 and/or BioA enzyme at increased levels compared to an unmodified plant.

In one embodiment, the plant or part thereof may be modified to comprise both a BIO3-BIO1 and a BioA enzyme wherein the BioA enzyme is overexpressed and wherein the BIO3-BIO1 enzyme comprises one or more mutations which provide the plant or part thereof with increased resistance to a compound which inhibits the biotin synthesis pathway relative to an unmodified plant.

In one embodiment, the plant has been transformed with said recombinant polynucleotide. Suitable means of transformation are described hereinbelow.

The transformed parts of plants, transformed plant cells or a transformed plant protoplasts as described herein may be regenerated to produce a modified plant as described herein.

When adequate numbers of transformed cells or protoplasts containing a recombinant BIO3-BIO1 and/or BioA enzyme are obtained, the cells can be cultured, then regenerated into whole plants. “Regeneration” refers to the process of growing a plant from a plant cell (for example, plant protoplast or explant). Such regeneration techniques rely on manipulation of certain phytohormones in a tissue culture growth medium, typically relying on a biocide and/or herbicide marker that has been introduced together with the desired nucleotide sequences. Choice of methodology for the regeneration step is not critical. See, for example, Ammirato et al., Handbook of Plant Cell Culture—Crop Species. Macmillan Publ. Co. (1984); Shimamoto et al., Nature 338:274-276 (1989); Fromm, UCLA Symposium on Molecular Strategies for Crop Improvement, Apr. 16-22, 1990. Keystone, Colo. (1990); Vasil et al., Bio/Technology 8:429-434 (1990); Vasil et al., Bio/Technology 10:667-674 (1992); Hayashimoto, Plant Physiol. 93:857-863 (1990); and Datta et al., Bio-technology 8:736-740 (1990). Such regeneration techniques are described generally in Klee et al., Ann. Rev. Plant Phys. 38:467-486 (1987).

### Modified BIO3-BIO1 Enzyme

As described above, in some embodiments, the BIO3-BIO1 enzyme may be modified with one or more mutations. Suitably the one or more mutations provide the enzyme, and therefore the plant, with increased resistance to a compound which inhibits the biotin synthesis pathway relative to an unmodified plant. In another aspect of the invention there is also provided a modified BIO3-BIO1 enzyme.

In some aspects and embodiments of the invention, the BIO3-BIO1 enzyme may be modified, suitably it may comprise one or more modifications, suitably one or more mutations. Suitably the BIO3-BIO1 enzyme may comprise one or more mutations in one or more of the motifs 1 to 14, or 17 identified above. By mutation, it is meant any substitution, deletion or insertion of 1, 2, 3, 4, 5, 6, 7, 8, 9, 10 or more amino acids. Mutated BIO3-BIO1 enzymes of the invention may comprise such a mutation at one or more positions of any of motifs 1 to 14, and 17. In one embodiment, the BIO3-BIO1 enzyme of the invention may comprise a substitution mutation at one or more of positions of any of motifs 1 to 14 and 17. Suitable positions of each of motifs 1 to 14 which may be modified are defined hereinbelow.

As used herein with reference to amino acid sequences, the use of “/” between amino acid residues is used to denote “or”. For example, the substitution of an amino acid residue with amino acid residues X orY may be denoted as “X/Y”. In the context of a motif, modified residue positions may be denoted by including the possible substituents of the position in brackets i.e. amino acid residue two of WW substituted with X may be denoted as “(W/X)”. In addition, in relation to motifs as used herein “bold” type face is used to denote substituents at a position of the motif.

### Motif 1

Suitably the third residue (position 3) of motif 1 may be substituted. For example, by an A/E substitution. In some examples, residue P347 of SEQ ID NO:1, or a corresponding residue thereto, is substituted with an A or an E amino acid. For example, the BIO3-BIO1 enzyme may comprise the motif:

(SEQ ID NO: 222)
W; (H/Y/W); (A/E); F; (A/Q/S/T); Q; (H/Q/V);
X; X; X

Suitably residue 4 (i.e. position 4) of motif 1 may be substituted. For example, by a A/C/D/E/I/K/M/N/Q/S/TV substitution. In some examples, residue F348 of SEQ ID NO:1, or a corresponding residue thereto, is substituted with an A/C/D/E/I/K/M/N/Q/S/TV amino acid. For example, the BIO3-BIO1 enzyme may comprise the motif:

(SEQ ID NO: 223)
W;(H/Y/W);P;(A/C/D/E/I/K/M/N/Q/S/T/V);(A/Q/S/T);Q;
(H/Q/V);X;X;X

Suitably residue 6 (i.e. position 6) of motif 1 may be substituted. For example, by an H/S substitution. In some examples, residue Q350 of SEQ ID NO:1, or a corresponding position thereto is substituted with an H/S amino acid. For example, the BIO3-BIO1 enzyme may comprise the motif:

(SEQ ID NO: 224)
W;(H/Y/W);P;F;(A/Q/S/T);(H/S);(H/Q/V);X;X;X

Suitably residue 10 (i.e. position 10) of motif 1 may be substituted. For example, by an A/E/L/N/T substitution. In some examples, residue V354 of SEQ ID NO:1, or a corresponding position thereto is substituted with an A/E/L/N/T amino acid. For example, the BIO3-BIO1 enzyme may comprise the motif:

(SEQ ID NO: 225)

W;(H/Y/W);P;F;(A/Q/S/T);Q;(H/Q/V);X;X;(A/E/L/N/T)

In one embodiment, therefore, the above substitutions may be present in one motif, the modified BIO3-BIO1 enzyme may comprise the modified motif 1:

| (SEQ ID NO: 226) | |
|---|---|
| | W;(H/Y/W);(A/E);(A/C/D/E/I/K/M/N/Q/S/T/V); |
| | (A/Q/S/T);(H/S);(H/Q/V);X;X;(A/E/L/N/T) |

### Motif 2

Suitably the ninth amino acid (position 9) of motif 2 may be substituted. For example, by an L substitution. In some examples, residue F370 of SEQ ID NO:1, or a corresponding residue thereto, is substituted with an L amino acid. For example, the BIO3-BIO1 enzyme may comprise the motif:

(SEQ ID NO: 227)
(I/L/V);(D/E);(S/G);(R/A);X;(A/D/G/K);(E/D/N);X;L

### Motif 3

Suitably the fourth amino acid (position 4) of motif 3 may be substituted. For example, by an D/M/T substitution. In some examples, residue C388 of SEQ ID NO:1, or a corresponding residue thereto, is substituted with an D/M/T amino acid. For example, the BIO3-BIO1 enzyme may comprise the motif:

(SEQ ID NO: 228)
(F/I/L/V/Y);D;(A/G);(D/M/T);(A/G/S);S;W;W;(T/S/V);
(I/Q)

Suitably the fifth amino acid (position 5) of motif 3 may be substituted. For example, by an F substitution. In some examples, residue A389 of SEQ ID NO:1, or a corresponding residue thereto, is substituted with an F amino acid. For example, the BIO3-BIO1 enzyme may comprise the motif:

(SEQ ID NO: 229)
(F/I/L/V/Y);D;(A/G);(C/I/P/S);F;S;W;W;(T/S/V);(I/Q)

Suitably the sixth amino acid (position 6) of motif 3 may be substituted. For example, by a C substitution. In some examples, residue S390 of SEQ ID NO:1, or a corresponding residue thereto, is substituted with a C amino acid. For example, the BIO3-BIO1 enzyme may comprise the motif:

(SEQ ID NO: 230)

(F/I/L/V/Y);D;(A/G);(C/I/P/S);(A/G/S);C;W;W;
(T/S/V);(I/Q)

Suitably the seventh amino acid (position 7) of motif 3 may be substituted. For example, by a F/L/M substitution. In some examples, residue W391 of SEQ ID NO:1, or a corresponding residue thereto, is substituted with a F/L/M amino acid. For example, the BIO3-BIO1 enzyme may comprise the motif:

(SEQ ID NO: 231)
(F/I/L/V/Y);D;(A/G);(C/I/P/S);(A/G/S);S;(F/L/M);W;
(T/S/V);(I/Q)

Suitably the eighth amino acid (position 8) of motif 3 may be substituted. For example, by a A/C/D/G/M/S substitution. In some examples, residue W392 of SEQ ID NO:1, or a corresponding residue thereto, is substituted with a A/C/D/G/M/S amino acid. For example, the BIO3-BIO1 enzyme may comprise the motif:

| (SEQ ID NO: 232) | |
|---|---|
| | (F/I/L/V/Y);D;(A/G);(C/I/P/S);(A/G/S);S;W; |
| | (A/C/D/G/M/S);(T/S/V);(I/Q) |

Suitably the ninth amino acid (position 9) of motif 3 may be substituted. For example, by a V substitution. In some examples, residue T393 of SEQ ID NO:1, or a corresponding residue thereto, is substituted with a V amino acid. For example, the BIO3-BIO1 enzyme may comprise the motif:

(SEQ ID NO: 233)

(F/I/L/V/Y);D;(A/G);(C/I/P/S);(A/G/S);S;W;W;V;
(I/Q)

In one embodiment, therefore, the above substitutions may be present in one motif, the modified BIO3-BIO1 enzyme may comprise the modified motif 3:

| (SEQ ID NO: 234) | |
|---|---|
| | (F/I/L/V/Y);D;(A/G);(D/M/T);F;C;(F/L/M); |
| | (A/C/D/G/M/S);V;(I/Q) |

### Motif4

Suitably the fifth amino acid (position 5) of motif 4 may be substituted. For example, by an I substitution. In some examples, residue M419 of SEQ ID NO:1, or a corresponding residue thereto, is substituted with an I amino acid. For example, the BIO3-BIO1 enzyme may comprise the motif:

(SEQ ID NO: 235)

(F/Y);(G/D);(H/Q);(A/I/V);I;(F/L/Y);(A/L/P);
(E/G/H/L/N/S/T)

Suitably the sixth amino acid (position 6) of motif 4 may be substituted. For example, by an I substitution. In some examples, residue F420 of SEQ ID NO:1, or a corresponding residue thereto, is substituted with an I amino acid. For example, the BIO3-BIO1 enzyme may comprise the motif:

(SEQ ID NO: 236)

(F/Y);(G/D);(H/Q);(A/I/V);(M/I/L);I;(A/L/P);
(E/G/H/L/N/S/T)

Suitably the seventh amino acid (position 7) of motif 4 may be substituted. For example, by an A/E/G/L/W substitution. In some examples, residue P421 of SEQ ID NO:1, or a corresponding residue thereto, is substituted with an A/E/G/L/W amino acid. For example, the BIO3-BIO1 enzyme may comprise the motif:

| (SEQ ID NO: 237) | |
|---|---|
| | (F/Y);(G/D);(H/Q);(A/I/V);(M/I/L);(F/L/Y); |
| | (A/E/G/L/W);(E/G/H/L/N/S/T) |

In one embodiment, therefore, the modified BIO3-BIO1 enzyme may comprise the modified motif 4:

(SEQ ID NO: 238)
(F/Y); (G/D); (H/Q); (A/I/V); I; I; (A/E/G/L/W);
(E/G/H/L/N/S/T)

### Motif 5

Suitably the fifth amino acid (position 5) of motif 5 may be substituted. For example, by an A substitution. In some examples, residue S494 of SEQ ID NO:1, or a corresponding residue thereto, is substituted with an A amino acid. For example, the BIO3-BIO1 enzyme may comprise the motif:

(SEQ ID NO: 239)
(A/G/T); (F/L/T/Q/V); X; (G/N/D/E/R); A; Y; H; G;
D; T; (L/I/M); (G/S); (A/C/S/T/V); (M/L/T);
(D/E/N); X; (A/E/K/Q/R/S/T); (A/E/I/Q/V/T);
(E/G/I/K/P/S); (C/E/N/S/T); X; (F/T/Y); (M/N/S/T);
X

Suitably the seventh amino acid (position 7) of motif 5 may be substituted. For example, by an S substitution. In some examples, residue H496 of SEQ ID NO:1, or a corresponding residue thereto, is substituted with an S amino acid. For example, the BIO3-BIO1 enzyme may comprise the motif:

(SEQ ID NO: 240)
(A/G/T); (F/L/T/Q/V); X; (G/N/D/E/R); (S/C/G); Y;
S; G; D; T; (L/I/M); (G/S); (A/C/S/T/V); (M/L/T);
(D/E/N); X; (A/E/K/Q/R/S/T); (A/E/I/Q/V/T);
(E/G/I/K/P/S); (C/E/N/S/T); X; (F/T/Y); (M/N/S/T);
X

Suitably the seventeenth amino acid (position 17) of motif 5 may be substituted. For example, by an A substitution. In some examples, residue Q506 of SEQ ID NO:1, or a corresponding residue thereto, is substituted with an A amino acid. For example, the BIO3-BIO1 enzyme may comprise the motif:

(SEQ ID NO: 241)
(A/G/T); (F/L/T/Q/V); X; (G/N/D/E/R); (S/C/G); Y;
H; G; D; T; (L/I/M); (G/S); (A/C/S/T/V); (M/L/T);
(D/E/N); X; A; (A/E/I/Q/V/T); (E/G/I/K/P/S);
(C/E/N/S/T); X; (F/T/Y); (M/N/S/T); X

Suitably the eighteenth amino acid (position 18) of motif 5 may be substituted. For example, by an K/S substitution. In some examples, residue A507 of SEQ ID NO:1, or a corresponding residue thereto, is substituted with an K/S amino acid. For example, the BIO3-BIO1 enzyme may comprise the motif:

(SEQ ID NO: 242)
(A/G/T); (F/L/T/Q/V); X; (G/N/D/E/R); (S/C/G); Y;
H; G; D; T; (L/I/M); (G/S); (A/C/S/T/V); (M/L/T);
(D/E/N); X; (A/E/K/Q/R/S/T); (K/S); (E/G/I/K/P/S);
(C/E/N/S/T); X; (F/T/Y); (M/N/S/T); X

Suitably the nineteenth amino acid (position 19) of motif 5 may be substituted. For example, by an L/T substitution, or a deletion. In some examples, residue P508 of SEQ ID NO:1, or a corresponding residue thereto, is substituted with an L/T amino acid, or is deleted. For example, the BIO3-BIO1 enzyme may comprise the motif:

(SEQ ID NO: 243)
(A/G/T); (F/L/T/Q/V); X; (G/N/D/E/R); (S/C/G); Y;
H; G; D; T; (L/I/M); (G/S); (A/C/S/T/V); (M/L/T);
(D/E/N); X; (A/E/K/Q/R/S/T); (A/E/I/Q/V/T);
(DELETION/L/T); (C/E/N/S/T); X; (F/T/Y);
(M/N/S/T); X

Suitably the twentieth amino acid (position 20) of motif 5 may be substituted. For example, by an A/C/D/E/F/G/H/I/K/L/M/N/Q/R/S/T/V/W/Y substitution. In some examples, residue S509 of SEQ ID NO:1, or a corresponding residue thereto, is substituted with an A/C/D/E/F/G/H/I/K/L/M/N/Q/R/S/T/V/W/Y amino acid. For example, the BIO3-BIO1 enzyme may comprise the motif:

(SEQ ID NO: 244)
(A/G/T); (F/L/T/Q/V); X; (G/N/D/E/R); (S/C/G);
Y; H; G; D; T; (L/I/M); (G/S); (A/C/S/T/V);
(M/L/T); (D/E/N); X; (A/E/K/Q/R/S/T);
(A/E/I/Q/V/T); (E/G/I/K/P/S);
(A/C/D/E/F/G/H/I/K/L/M/N/Q/R/S/T/V/W/Y); X;
(F/T/Y); (M/N/S/T); X

Suitably the twenty-first amino acid (position 21) of motif 5 may be substituted. For example, by an A/C/E/L/Q/V substitution. In some examples, residue P510 of SEQ ID NO:1, or a corresponding residue thereto, is substituted with an A/C/E/L/Q/V amino acid. For example, the BIO3-BIO1 enzyme may comprise the motif:

(SEQ ID NO: 245)
(A/G/T); (F/L/T/Q/V); X; (G/N/D/E/R); (S/C/G);
Y; H; G; D; T; (L/I/M); (G/S); (A/C/S/T/V);
(M/L/T); (D/E/N); X; (A/E/K/Q/R/S/T);
(A/E/I/Q/V/T); (E/G/I/K/P/S); (C/E/N/S/T);
(A/C/E/L/Q/V); (F/T/Y); (M/N/S/T); X

Suitably the twenty-second amino acid (position 22) of motif 5 may be substituted. For example, by an C/D/E/F/H/I/K/M/P/Q/R/V/W substitution. In some examples, residue Y511 of SEQ ID NO:1, or a corresponding residue thereto, is substituted with an C/D/E/F/H/I/K/M/P/Q/R/V/W amino acid. For example, the BIO3-BIO1 enzyme may comprise the motif:

(SEQ ID NO: 246)
(A/G/T); (F/L/T/Q/V); X; (G/N/D/E/R); (S/C/G); Y;
H; G; D; T; (L/I/M); (G/S); (A/C/S/T/V); (M/L/T);
(D/E/N); X; (A/E/K/Q/R/S/T); (A/E/I/Q/V/T);
(E/G/I/K/P/S); (C/E/N/S/T); X;
(C/D/E/F/H/I/K/M/P/Q/R/V/W); (M/N/S/T); X

Suitably the twenty-third amino acid (position 23) of motif 5 may be substituted. For example, by an C/D/G/I/N/Q/R/V/W substitution. In some examples, residue T512 of SEQ ID NO:1, or a corresponding residue thereto, is substituted with an C/D/G/I/N/Q/R/V/W amino acid. For example, the BIO3-BIO1 enzyme may comprise the motif:

(SEQ ID NO: 247)
(A/G/T); (F/L/T/Q/V); X; (G/N/D/E/R); (S/C/G); Y;
H; G; D; T; (L/I/M); (G/S); (A/C/S/T/V); (M/L/T);
(D/E/N); X; (A/E/K/Q/R/S/T); (A/E/I/Q/V/T);
(E/G/I/K/P/S); (C/E/N/S/T); X; (F/T/Y);
(C/D/G/I/N/Q/R/V/W); X

Suitably the twenty-fourth amino acid (position 13) of motif 5 may be substituted. For example, by an A/L/P substitution, or a deletion. In some examples, residue G513 of SEQ ID NO:1, or a corresponding residue thereto, is substituted with an A/L/P amino acid, or is deleted. For example, the BIO3-BIO1 enzyme may comprise the motif:

(SEQ ID NO: 248)
(A/G/T); (F/L/T/Q/V); X; (G/N/D/E/R); (S/C/G); Y;
H; G; D; T; (L/I/M); (G/S); (A/C/S/T/V); (M/L/T);
(D/E/N); X; (A/E/K/Q/R/S/T); (A/E/I/Q/V/T);
(E/G/I/K/P/S); (C/E/N/S/T); X; (F/T/Y); (M/N/S/T);
(DELETION/A/L/P)

In one embodiment, therefore, the above substitutions may be present in one motif, the modified BIO3-BIO1 enzyme may comprise the modified motif 5:

(SEQ ID NO: 249)
(A/G/T); (F/L/T/Q/V); X; (G/N/D/E/R); A; Y; S; G;
D; T; (L/I/M); (G/S); (A/C/S/T/V); (M/L/T);
(D/E/N); X; A; (K/S); (DELETION/L/T);
(A/C/D/E/F/G/H/I/K/L/M/N/Q/R/S/T/V/W/Y);
(A/C/E/L/Q/V); (C/D/E/F/H/I/K/M/P/Q/R/V/W);
(C/D/G/I/N/Q/R/V/W); (DELETION/A/L/P)

### Motif 6

Suitably the first amino acid (position 1) of motif 6 may be substituted. For example, by an C substitution. In some examples, residue Q516 of SEQ ID NO:1, or a corresponding residue thereto, is substituted with an C amino acid. For example, the BIO3-BIO1 enzyme may comprise the motif:

(SEQ ID NO: 250)
C; (E/H/I/Q/V/T); (D/E/H/P); (S/W); (F/H/Y); X;
(G/P/Q/R/S); (E/K/Q/R/W)

Suitably the second amino acid (position 2) of motif 6 may be substituted. For example, by an D/F/H/I/M/T/W/Y substitution. In some examples, residue Q517 of SEQ ID NO:1, or a corresponding residue thereto, is substituted with an D/F/H/I/M/T/W/Y amino acid. For example, the BIO3-BIO1 enzyme may comprise the motif:

(SEQ ID NO: 251)
(A/E/K/Q/R/S/T); (D/F/H/I/M/T/W/Y); (D/E/H/P);
(S/W); (F/H/Y); X; (G/P/Q/R/S); (E/K/Q/R/W)

Suitably the fifth amino acid (position 5) of motif 6 may be substituted. For example, by an N/W substitution. In some examples, residue Y520 of SEQ ID NO:1, or a corresponding residue thereto, is substituted with an N/W amino acid. For example, the BIO3-BIO1 enzyme may comprise the motif:

(SEQ ID NO: 252)
(A/E/K/Q/R/S/T); (E/H/I/Q/V/T); (D/E/H/P); (S/W);
(N/W); X;(G/P/Q/R/S); (E/K/Q/R/W)

In one embodiment, therefore, the above substitutions may be present in one motif, the modified BIO3-BIO1 enzyme may comprise the modified motif 6:

(SEQ ID NO: 253)
C; (D/F/H/I/M/T/W/Y); (D/E/H/P); (S/W); (N/W); X; (G/P/Q/R/S); (E/K/Q/R/W)

### Motif 7

Suitably the fourth amino acid (position 4) of motif 7 may be substituted. For example, by an A substitution. In some examples, residue P529 of SEQ ID NO:1, or a corresponding residue thereto, is substituted with an A amino acid. For example, the BIO3-BIO1 enzyme may comprise the motif:

(SEQ ID NO: 254)
(F/S/W); (F/I/L/P); X; A; (P/S); X; (I/L/M/T/V)

### Motif 8

Suitably the first amino acid (position 1) of motif 8 may be substituted. For example, by an A/E/I substitution. In some examples, residue G608 of SEQ ID NO:1, or a corresponding residue thereto, is substituted with an A/E/I amino acid. For example, the BIO3-BIO1 enzyme may comprise the motif:

(SEQ ID NO: 255)
(A/E/I); (A/S); (A/G); G; M; X; (F/M/L); (A/C/I/V)

Suitably the second amino acid (position 2) of motif 8 may be substituted. For example, by an C/F/H/I/K/M/N/R/T/V/W/Y substitution. In some examples, residue A609 of SEQ ID NO:1, or a corresponding residue thereto, is substituted with an C/F/H/I/K/M/N/R/T/V/W/Y amino acid. For example, the BIO3-BIO1 enzyme may comprise the motif:

(SEQ ID NO: 256)
(A/G); (C/F/H/I/K/M/N/R/T/V/W/Y); (A/G); G; M; X; (F/M/L); (A/C/I/V)

Suitably the third amino acid (position 3) of motif 8 may be substituted. For example, by an H substitution. In some examples, residue G610 of SEQ ID NO:1, or a corresponding residue thereto, is substituted with an H amino acid. For example, the BIO3-BIO1 enzyme may comprise the motif:

(SEQ ID NO: 257)
(A/G); (A/S); H; G; M; X; (F/M/L); (A/C/I/V)

Suitably the fifth amino acid (position 5) of motif 8 may be substituted. For example, by an L substitution. In some examples, residue M612 of SEQ ID NO:1, or a corresponding residue thereto, is substituted with an L amino acid. For example, the BIO3-BIO1 enzyme may comprise the motif:

(SEQ ID NO: 258)
(A/G); (A/S); (A/G); G; L; X; (F/M/L); (A/C/I/V)

In one embodiment, the above substitutions may be present in one motif, therefore, the modified BIO3-BIO1 enzyme may comprise the modified motif 8:

(SEQ ID NO: 259)
(A/E/I); (C/F/H/I/K/M/N/R/T/V/W/Y); H; G; L; X; (F/M/L); (A/C/I/V)

### Motif 9

Suitably the fourth amino acid (position 4) of motif 9 may be substituted. For example, by an A/C/S substitution. In some examples, residue G700 of SEQ ID NO:1, ora corresponding residue thereto, is substituted with an A/C/S amino acid. For example, the BIO3-BIO1 enzyme may comprise the motif:

(SEQ ID NO: 260)
L; L; H; (A/C/S); H; S; (F/Y); (N/S/T); (A/G/T); (H/N/Y)

Suitably the eighth amino acid (position 8) of motif 9 may be substituted. For example, by an H/P substitution. In some examples, residue S704 of SEQ ID NO:1, or a corresponding residue thereto, is substituted with an H/P amino acid. For example, the BIO3-BIO1 enzyme may comprise the motif:

(SEQ ID NO: 261)
L; L; H; (G/R); H; S; (F/Y); (H/P); (A/G/T);
(H/N/Y)

In one embodiment, therefore, the above substitutions may be present in one motif, the modified BIO3-BIO1 enzyme may comprise the modified motif 9:

(SEQ ID NO: 262)
L; L; H; (A/C/S); H; S; (F/Y); (H/P); (A/G/T); (H/N/Y)

### Motif 10

Suitably the fifth amino acid (position 5) of motif 10 may be substituted. For example, by an S/K substitution. In some examples, residue R756 of SEQ ID NO:1, or a corresponding residue thereto, is substituted with an S/K amino acid. For example, the BIO3-BIO1 enzyme may comprise the motif:

(SEQ ID NO: 263)
(A/E/G/H/K/P/Q/S/V); (A/C/G/K/N/Q/R/S/T/V/Y); (H/I/L/V); (A/D/E/H/K/L/N/Q/R/S);
(S/K); (A/C/I/L/T/V); (F/I/M/T/V/W); (A/D/G/N/S/V; (I/L/M/Q/V)

### Motif 11

Suitably the second amino acid (position 2) of motif 11 may be substituted. For example, by an S substitution. In some examples, residue L786 of SEQ ID NO:1, or a corresponding residue thereto, is substituted with an S amino acid. For example, the BIO3-BIO1 enzyme may comprise the motif:

(SEQ ID NO: 264)
(A/I/L/V/Y); S; (A/D/E/I/K/L/M/N/T/Q/R/S); (A/D/E/F/H/K/M/N/Q/R/S/T/V/Y); (F/L);
(A/H/K/L/M/R/S/T/Y); X; X; (F/G)

Suitably the sixth amino acid (position 6) of motif 11 may be substituted. For example, by a C substitution. In some examples, residue L790 of SEQ ID NO:1, or a corresponding residue thereto, is substituted with a C amino acid. For example, the BIO3-BIO1 enzyme may comprise the motif:

(SEQ ID NO: 265)
(A/I/L/V/Y); (A/I/L/N/Q/R/V); (A/D/E/I/K/L/M/N/T/Q/R/S); (A/D/E/F/H/K/
M/N/Q/R/S/T/V/Y); (F/L); C; X; X; (F/G)

In one embodiment, therefore, the above substitutions may be present in one motif, the modified BIO3-BIO1 enzyme may comprise the modified motif 11:

(SEQ ID NO: 266)
(A/I/L/V/Y); S; (A/D/E/I/K/L/M/N/T/Q/R/S); (A/D/E/F/H/K/M/N/Q/R/S/T/V/Y); (F/L);
C; X; X; (F/G)

### Motif 12

Suitably the fourth amino acid (position 4) of motif 12 may be substituted. For example, by a Q substitution. In some examples, residue R797 of SEQ ID NO:1, or a corresponding residue thereto, is substituted with a Q amino acid. For example, the BIO3-BIO1 enzyme may comprise the motif:

(SEQ ID NO: 267)
(A/I/L/M/N/V); (F/H/L/Q/Y); (A/C/E/I/L/M/S/T); Q; (A/I/P/S/V); L; G; (D/K/N; I/T/V);
(F/I/L/M/V); Y

Any combination of any number of the variable positions of any of the motifs 1 to 12 may be changed according to design or requirement.

As such, a modified BIO3-BIO1 enzyme of the invention may comprise a motif according to any of SEQ ID NOs 222 to 267. As such, a modified BIO3-BIO1 enzyme of the invention may comprise more than one motif according to any of SEQ ID NOs 222 to 267, in any combination. As such, a modified BIO3-BIO1 enzyme of the invention may comprise each of the motifs according to SEQ ID NOs 222 to 267. In particular, the modified BIO3-BIO1 enzyme of the invention may comprise each of the motifs according to SEQ ID Nos 226, 227, 234, 238, 249, 253, 254, 259, 262, 263, 266, and 267.

As such, a modified BIO3-BIO1 enzyme of the invention may comprise a sequence according to any of SEQ ID NO:1-14, 271-276 and 319, (or SEQ ID NO: 294 to 310 and 320), or a sequence having 30%, 35%, 40%, 45%, 50%, 55%, 60%, 65%, 70%, 75%, 80%, 85%, 90%, 91%, 92%, 93%, 94%, 95%, 96%, 97%, 98% or 99% thereto, and comprising a motif according to any of SEQ ID NOs 222 to 267. As such, a modified BIO3-BIO1 enzyme of the invention may comprise a sequence according to any of SEQ ID NO:1-14, 271-276 and 319, (or SEQ ID NO: 294 to 310 and 320), or a sequence having 30%, 35%, 40%, 45%, 50%, 55%, 60%, 65%, 70%, 75%, 80%, 85%, 90%, 91%, 92%, 93%, 94%, 95%, 96%, 97%, 98% or 99% thereto, and more than one motif according to any of SEQ ID NOs 222 to 267, in any combination. As such, a modified BIO3-BIO1 enzyme of the invention may comprise a sequence according to any of SEQ ID NO:1-14, 271-276 and 319, (or SEQ ID NO: 294 to 310 and 320), or a sequence having 30%, 35%, 40%, 45%, 50%, 55%, 60%, 65%, 70%, 75%, 80%, 85%, 90%, 91%, 92%, 93%, 94%, 95%, 96%, 97%, 98% or 99% thereto, and each of the motifs according to SEQ ID Nos 222 to 267. In particular, the modified BIO3-BIO1 enzyme of the invention may comprise a sequence according to any of SEQ ID NO:1-14, 271-276 and 319, (or SEQ ID NO: 294 to 310 and 320), or a sequence having 30%, 35%, 40%, 45%, 50%, 55%, 60%, 65%, 70%, 75%, 80%, 85%, 90%, 91%, 92%, 93%, 94%, 95%, 96%, 97%, 98% or 99% thereto, and each of the motifs according to SEQ ID Nos 226, 227, 234, 238, 249, 253, 254, 259, 262, 263, 266, and 267.

The modified BIO3-BIO1 enzyme may comprise an amino acid sequence that has at least 30% sequence identity to an amino acid sequence according to SEQ ID NO: 1, wherein positions 347 to 797 thereof, or corresponding positions thereto, include at least one mutation. Suitably wherein positions 347 to 370, 388 to 393, 419 to 421, 506 to 529, 608 to 612, 700 to 704, 756 to 797 of SEQ ID NO:1, or corresponding positions thereto, include at least one mutation. Suitably wherein positions 347 to 354, 370, 388 to 393, 419 to 421, 506 to 513, 516 to 520, 529, 608 to 612, 700 to 704, 756, 786 to 790, 797 of SEQ ID NO:1, or corresponding positions thereto, include at least one mutation.

Other variants of such an enzyme may comprise an amino acid sequence of at least 35%, at least 40%, at least 45%, at least 50%, at least 55%, at least 60%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 1, wherein positions 347 to 797 thereof, or corresponding positions thereto, include at least one mutation. Suitably wherein positions 347 to 370, 388 to 393, 419 to 421, 506 to 529, 608 to 612, 700 to 704, 756 to 797 or corresponding positions thereto, include at least one mutation. Suitably wherein positions 347 to 354, 370, 388 to 393, 419 to 421, 506 to 513, 516 to 520, 529, 608 to 612, 700 to 704, 756, 786 to 790, 797 or corresponding positions thereto, include at least one mutation.

A modified BIO3-BIO1 enzyme of the invention may comprise an amino acid sequence according to any of SEQ ID NO: 1-14, 271-276 and 319, (or SEQ ID NO: 294 to 310 and 320), wherein positions 347 to 797 as defined in relation to SEQ ID NO:1, or corresponding positions thereto in any of SEQ ID NOs 2-14, 271-276 and 319, include at least one mutation. Suitably wherein positions 347 to 370, 388 to 393, 419 to 421, 506 to 529, 608 to 612, 700 to 704, 756 to 797 as defined in relation to SEQ ID NO:1, or corresponding positions thereto in any of SEQ ID NOs 2-14, 271-276 and 319, (or SEQ ID NO: 294 to 310 and 320), include at least one mutation. Suitably wherein positions 347 to 354, 370, 388 to 393, 419 to 421, 506 to 513, 516 to 520, 529, 608 to 612, 700 to 704, 756, 786 to 790, 797 as defined in relation to SEQ ID NO:1, or corresponding positions thereto in any of SEQ ID NOs 2-14, 271-276 and 319, (or SEQ ID NO: 294 to 310 and 320), include at least one mutation.

A modified BIO3-BIO1 enzyme of the invention may comprise an amino acid sequence according to SEQ ID NO:1 (Arabidopsis thaliana BIO3-BIO1) wherein positions 347 to 797 thereof include at least one mutation. Suitably wherein positions 347 to 370, 388 to 393, 419 to 421, 506 to 529, 608 to 612, 700 to 704, 756 to 797 of SEQ ID NO:1 include at least one mutation. Suitably wherein positions 347 to 354, 370, 388 to 393, 419 to 421, 506 to 513, 516 to 520, 529, 608 to 612, 700 to 704, 756, 786 to 790, 797 of SEQ ID NO:1 include at least one mutation.

Positions 347 to 797 of SEQ ID NO: 1 correspond to positions 332 to 785 of SEQ ID NO: 2 (which relates to Zea mays BIO3-BIO1). As such, the modified BIO3-BIO1 enzyme may have an amino acid sequence according to SEQ ID NO: 2 wherein positions 332 to 785 thereof include at least one mutation.

The modified BIO3-BIO1 enzyme may comprise an amino acid sequence that has at least 70% sequence identity to an amino acid sequence according to SEQ ID NO: 2, wherein positions 332 to 785 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 2 wherein positions 332 to 785 thereof include at least one mutation as described herein.

Positions 347 to 797 of SEQ ID NO: 1 correspond to positions 290 to 808 of SEQ ID NO: 3 (which relates to Nannochloropsis gaditana BIO3-BIO1). As such, the modified BIO3-BIO1 enzyme may have an amino acid sequence according to SEQ ID NO: 3 wherein positions 290 to 808 thereof include at least one mutation.

The modified BIO3-BIO1 enzyme may comprise an amino acid sequence that has at least 70% sequence identity to an amino acid sequence according to SEQ ID NO: 3, wherein positions 290 to 808 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 3 wherein positions 290 to 808 thereof include at least one mutation as described herein.

Positions 347 to 797 of SEQ ID NO: 1 correspond to positions 333 to 791 of SEQ ID NO: 7 (which relates to Setaria italica BIO3-BIO1). As such, the modified BIO3-BIO1 enzyme may have an amino acid sequence according to SEQ ID NO: 7 wherein positions 333 to 791 thereof include at least one mutation.

The modified BIO3-BIO1 enzyme may comprise an amino acid sequence that has at least 70% sequence identity to an amino acid sequence according to SEQ ID NO: 7, wherein positions 333 to 791 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 7 wherein positions 333 to 791 thereof include at least one mutation as described herein.

Positions 347 to 797 of SEQ ID NO: 1 correspond to positions 343 to 799 of SEQ ID NO: 10 (which relates to Helianthus annuus BIO3-BIO1). As such, the modified BIO3-BIO1 enzyme may have an amino acid sequence according to SEQ ID NO: 10 wherein positions 343 to 799 thereof include at least one mutation.

The modified BIO3-BIO1 enzyme may comprise an amino acid sequence that has at least 70% sequence identity to an amino acid sequence according to SEQ ID NO: 10, wherein positions 343 to 799 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 10 wherein positions 343 to 799 thereof include at least one mutation as described herein.

Positions 347 to 797 of SEQ ID NO: 1 correspond to positions 365 to 822 of SEQ ID NO: 8 (which relates to Phoenix dactylifera BIO3-BIO1). As such, the modified BIO3-BIO1 enzyme may have an amino acid sequence according to SEQ ID NO: 8 wherein positions 365 to 822 thereof include at least one mutation.

The modified BIO3-BIO1 enzyme may comprise an amino acid sequence that has at least 70% sequence identity to an amino acid sequence according to SEQ ID NO: 8, wherein positions 365 to 822 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 8 wherein positions 365 to 822 thereof include at least one mutation as described herein.

Positions 347 to 797 of SEQ ID NO: 1 correspond to positions 359 to 815 of SEQ ID NO: 11 (which relates to Quercus robur BIO3-BIO1). As such, the modified BIO3-BIO1 enzyme may have an amino acid sequence according to SEQ ID NO: 11 wherein positions 359 to 815 thereof include at least one mutation.

The modified BIO3-BIO1 enzyme may comprise an amino acid sequence that has at least 70% sequence identity to an amino acid sequence according to SEQ ID NO: 11, wherein positions 359 to 815 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 11 wherein positions 359 to 815 thereof include at least one mutation as described herein.

Positions 347 to 797 of SEQ ID NO: 1 correspond to positions 373 to 778 of SEQ ID NO: 4 (which relates to Taxus chinensis BIO3-BIO1). As such, the modified BIO3-BIO1 enzyme may have an amino acid sequence according to SEQ ID NO: 4 wherein positions 373 to 778 thereof include at least one mutation.

The modified BIO3-BIO1 enzyme may comprise an amino acid sequence that has at least 70% sequence identity to an amino acid sequence according to SEQ ID NO: 4, wherein positions 373 to 778 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 4 wherein positions 373 to 778 thereof include at least one mutation as described herein.

Positions 347 to 797 of SEQ ID NO: 1 correspond to positions 331 to 817 of SEQ ID NO: 9 (which relates to Ostreococcus tauri BIO3-BIO1). As such, the modified BIO3-BIO1 enzyme may have an amino acid sequence according to SEQ ID NO: 9 wherein positions 331 to 817 thereof include at least one mutation.

The modified BIO3-BIO1 enzyme may comprise an amino acid sequence that has at least 70% sequence identity to an amino acid sequence according to SEQ ID NO: 9, wherein positions 331 to 817 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 9 wherein positions 331 to 817 thereof include at least one mutation as described herein.

Positions 347 to 797 of SEQ ID NO: 1 correspond to positions 442 to 897 of SEQ ID NO: 5 (which relates to Physcomitrium patens BIO3-BIO1). As such, the modified BIO3-BIO1 enzyme may have an amino acid sequence according to SEQ ID NO: 5 wherein positions 442 to 897 thereof include at least one mutation.

The modified BIO3-BIO1 enzyme may comprise an amino acid sequence that has at least 70% sequence identity to an amino acid sequence according to SEQ ID NO: 5, wherein positions 442 to 897 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 5 wherein positions 442 to 897 thereof include at least one mutation as described herein.

Positions 347 to 797 of SEQ ID NO: 1 correspond to positions 323 to 871 of SEQ ID NO: 6 (which relates to Adiantum nelumboides BIO3-BIO1). As such, the modified BIO3-BIO1 enzyme may have an amino acid sequence according to SEQ ID NO: 6 wherein positions 323 to 871 thereof include at least one mutation.

The modified BIO3-BIO1 enzyme may comprise an amino acid sequence that has at least 70% sequence identity to an amino acid sequence according to SEQ ID NO: 6, wherein positions 323 to 871 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 6 wherein positions 323 to 871 thereof include at least one mutation as described herein.

Positions 347 to 797 of SEQ ID NO: 1 correspond to positions 262 to 754 of SEQ ID NO: 13 (which relates to Schizosaccharomyces japonicus BIO3-BIO1). As such, the modified BIO3-BIO1 enzyme may have an amino acid sequence according to SEQ ID NO: 13 wherein positions 262 to 754 thereof include at least one mutation.

The modified BIO3-BIO1 enzyme may comprise an amino acid sequence that has at least 70% sequence identity to an amino acid sequence according to SEQ ID NO: 13, wherein positions 262 to 754 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 13 wherein positions 262 to 754 thereof include at least one mutation as described herein.

Positions 347 to 797 of SEQ ID NO: 1 correspond to positions 269 to 733 of SEQ ID NO: 14 (which relates to Gibberella zeae BIO3-BIO1). As such, the modified BIO3-BIO1 enzyme may have an amino acid sequence according to SEQ ID NO: 14 wherein positions 269 to 733 thereof include at least one mutation.

The modified BIO3-BIO1 enzyme may comprise an amino acid sequence that has at least 70% sequence identity to an amino acid sequence according to SEQ ID NO: 14, wherein positions 269 to 733 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 14 wherein positions 269 to 733 thereof include at least one mutation as described herein.

Positions 347 to 797 of SEQ ID NO: 1 correspond to positions 303 to 732 of SEQ ID NO: 12 (which relates to Thraustotheca clavata BIO3-BIO1). As such, the modified BIO3-BIO1 enzyme may have an amino acid sequence according to SEQ ID NO: 12 wherein positions 303 to 732 thereof include at least one mutation.

The modified BIO3-BIO1 enzyme may comprise an amino acid sequence that has at least 70% sequence identity to an amino acid sequence according to SEQ ID NO: 12, wherein positions 303 to 732 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 12 wherein positions 303 to 732 thereof include at least one mutation as described herein.

Positions 347 to 797 of SEQ ID NO: 1 correspond to positions 329 to 788 of SEQ ID NO: 271 (which relates to Hordeum vulgare BIO3-BIO1). As such, the modified BIO3-BIO1 enzyme may have an amino acid sequence according to SEQ ID NO: 271 wherein positions 329 to 788 thereof include at least one mutation.

The modified BIO3-BIO1 enzyme may comprise an amino acid sequence that has at least 70% sequence identity to an amino acid sequence according to SEQ ID NO: 271, wherein positions 329 to 788 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 271 wherein positions 329 to 788 thereof include at least one mutation as described herein.

Positions 347 to 797 of SEQ ID NO: 1 correspond to positions 347 to 802 of SEQ ID NO: 272 (which relates to Brassica napus BIO3-BIO1). As such, the modified BIO3-BIO1 enzyme may have an amino acid sequence according to SEQ ID NO: 272 wherein positions 347 to 802 thereof include at least one mutation.

The modified BIO3-BIO1 enzyme may comprise an amino acid sequence that has at least 70% sequence identity to an amino acid sequence according to SEQ ID NO: 272, wherein positions 347 to 815 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 272 wherein positions 347 to 815 thereof include at least one mutation as described herein.

Positions 347 to 797 of SEQ ID NO: 1 correspond to positions 355 to 815 of SEQ ID NO: 273 (which relates to Gossypium hirsutum BIO3-BIO1). As such, the modified BIO3-BIO1 enzyme may have an amino acid sequence according to SEQ ID NO: 273 wherein positions 355 to 815 thereof include at least one mutation.

The modified BIO3-BIO1 enzyme may comprise an amino acid sequence that has at least 70% sequence identity to an amino acid sequence according to SEQ ID NO: 273, wherein positions 355 to 748 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 273 wherein positions 355 to 748 thereof include at least one mutation as described herein.

Positions 347 to 797 of SEQ ID NO: 1 correspond to positions 331 to 787 of SEQ ID NO: 274 (which relates to Oryza sativa BIO3-BIO1). As such, the modified BIO3-BIO1 enzyme may have an amino acid sequence according to SEQ ID NO: 274 wherein positions 331 to 787 thereof include at least one mutation.

The modified BIO3-BIO1 enzyme may comprise an amino acid sequence that has at least 70% sequence identity to an amino acid sequence according to SEQ ID NO: 274, wherein positions 331 to 787 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 274 wherein positions 331 to 787 thereof include at least one mutation as described herein.

Positions 347 to 797 of SEQ ID NO: 1 correspond to positions 330 to 783 of SEQ ID NO: 275 (which relates to Glycine max BIO3-BIO1). As such, the modified BIO3-BIO1 enzyme may have an amino acid sequence according to SEQ ID NO: 275 wherein positions 330 to 783 thereof include at least one mutation.

The modified BIO3-BIO1 enzyme may comprise an amino acid sequence that has at least 70% sequence identity to an amino acid sequence according to SEQ ID NO: 275, wherein positions 330 to 783 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 275 wherein positions 330 to 783 thereof include at least one mutation as described herein.

Positions 347 to 797 of SEQ ID NO: 1 correspond to positions 329 to 787 of SEQ ID NO: 276 (which relates to Triticum aestivum BIO3-BIO1). As such, the modified BIO3-BIO1 enzyme may have an amino acid sequence according to SEQ ID NO: 276 wherein positions 329 to 787 thereof include at least one mutation.

The modified BIO3-BIO1 enzyme may comprise an amino acid sequence that has at least 70% sequence identity to an amino acid sequence according to SEQ ID NO: 276, wherein positions 329 to 787 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 276 wherein positions 329 to 787 thereof include at least one mutation as described herein.

Positions 347 to 797 of SEQ ID NO: 1 correspond to positions P357, F358, Q360, V364, F380, C397, A398, S399, W400, W401, T402, M428, F429, P430, Q514, A515, P516, S517, P518, Y519, T520, S521, Q524, Q525, Y528, P537, A615, A616, G617, M619, G707, T711, R763, V794, R798, and R806 of SEQ ID NO: 319 (which relates to Selaginella moellendorffii BIO3-BIO1). As such, the modified BIO3-BIO1 enzyme may have an amino acid sequence according to SEQ ID NO: 319 wherein positions P357, F358, Q360, V364, F380, C397, A398, S399, W400, W401, T402, M428, F429, P430, Q514, A515, P516, S517, P518, Y519, T520, S521, Q524, Q525, Y528, P537, A615, A616, G617, M619, G707, T711, R763, V794, R798, and R806 thereof include at least one mutation.

The modified BIO3-BIO1 enzyme may comprise an amino acid sequence that has at least 70% sequence identity to an amino acid sequence according to SEQ ID NO: 319, wherein positions P357, F358, Q360, V364, F380, C397, A398, S399, W400, W401, T402, M428, F429, P430, Q514, A515, P516, S517, P518, Y519, T520, S521, Q524, Q525, Y528, P537, A615, A616, G617, M619, G707, T711, R763, V794, R798, and R806 include at least one mutation. Other variants of such an enzyme may comprise an amino acid sequence of at least 75%, at least 80%, at least 85%, at least 90%, at least 95%, at least 98% or at least 99% sequence identity to an amino acid sequence according to SEQ ID NO: 319 wherein positions P357, F358, Q360, V364, F380, C397, A398, S399, W400, W401, T402, M428, F429, P430, Q514, A515, P516, S517, P518, Y519, T520, S521, Q524, Q525, Y528, P537, A615, A616, G617, M619, G707, T711, R763, V794, R798, and R806 thereof include at least one mutation as described herein

In any aspect of the invention as described herein, a BIO3-BIO1 enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NOs 1 to 14, 271-276 and 319, (or SEQ ID NO: 294 to 310 and 320), herein, which may also be referred to as reference sequences.

In any aspect of the invention, a BIO3-BIO1 enzyme which has resistance to a compound which inhibits the biotin synthesis pathway may be a homologue of any of SEQ ID NOs 1 to 14, 271-276 and 319, (or SEQ ID NO: 294 to 310 and 320),. As used herein, “homologue” refers to a protein that is functionally equivalent i.e. has the same enzymatic activity as an enzyme having an amino acid sequence according to SEQ ID NO 1 to 14, 271-276 and 319, (or SEQ ID NO: 294 to 310 and 320), (i.e. acts as a BIO3-BIO1 enzyme as defined herein), but may have a limited number of amino acid substitutions, deletions, insertions or additions in the amino acid sequence. Homologues may have lower sequences identities, for example at least 20%, at least 25%, at least 30%, at least 35% or at least 40% or more sequence identity to a BIO3-BIO1 enzyme identified herein, but are capable of carrying out the same enzymatic reaction. The invention therefore includes any isoforms of BIO3-BIO1 enzymes and their mutations as defined herein.

“Identity” or “percent identity” refers to the degree of sequence variation between two given nucleic acid or amino acid sequences. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters. Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection. One example of an algorithm that is suitable for determining percent sequence identity and sequence similarity is the BLAST algorithm, which is described in Altschul et al., J. Mol. Biol.215: 403-410 (1990). Software for performing BLAST analyses is publicly available through the National Center for Biotechnology Information (on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighbourhood word score threshold (Altschul et al., J. Mol. Biol.215: 403-410 (1990)). These initial neighbourhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see, Henikoff & Henikoff, Proc. Natl. Acad. Sci. USA 89: 10915 (1989)). In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, In one embodiment less than about 0.01, and In one embodiment less than about 0.001.

Unless otherwise stated, “percent identity” as used herein refers to the value obtained using the Needleman and Wunsch algorithm ((1970) J. Mol. Biol. 48:443-453) implemented in the EMBOSS Needle alignment tool using default matrix files EBLOSUM62 for protein with default parameters (Gap Open=10, Gap Extend=0.5, End Gap Penalty=False, End Gap Open=10, End Gap Extend=0.5) or DNAfull for nucleic acids with default parameters (Gap Open=10, Gap Extend=0.5, End Gap Penalty=False, End Gap Open=10, End Gap Extend=0.5); or any equivalent program thereof. EMBOSS Needle is available, e.g., from EMBL-EBI such as at the following website: ebi.ac.uk/Tools/psa/emboss_needle/and as described in the following publication: “The EMBL-EBI search and sequence analysis tools APIs in 2019.” Madeira et al. Nucleic Acids Research, June 2019, 47(W1):W636-W641. The term “equivalent program” as used herein refers to any sequence comparison program that, for any two sequences in question, generates an alignment having identical nucleotide or amino acid residue matches and an identical percent sequence identity when compared to the corresponding alignment generated by EMBOSS Needle

A BIO3-BIO1 enzyme encoded by a nucleic acid or a BIO3-BIO1 enzyme of the invention may be a functional fragment of a BIO3-BIO1 enzyme as described herein. A “functional fragment” refers to a protein fragment that retains protein function. As such, a functional fragment of an BIO3-BIO1 enzyme is a fragment, portion or part of a BIO3-BIO1 protein that is capable of catalysing the conversion of KAPA into dethiobiotin.

The mutations defined herein may be located at a position ‘corresponding to’ an amino acid position listed in another BIO3-BIO1 or BioA enzyme. It is possible to compare BIO3-BIO1 or BioA polypeptides by sequence comparison and locating conserved regions that correspond to the amino acid positions listed, as is shown in FIG. 1 which provides an alignment of BIO3-BIO1 enzymes from various origins, or FIG. 3 which provides an alignment of BioA enzymes from various origins. The term “equivalent amino acids” or “corresponding amino acids” refers to amino acids in a sequence of interest, which correspond to those amino acids of an identified reference sequence, typically herein the reference sequence is SEQ ID NO:1 for BIO3-BIO1 enzymes, or SEQ ID NO:159 for BioA enzymes. A region of equivalent or corresponding amino acids may be determined by aligning the amino acid sequences of the proteins from the different species, using an alignment program such as BLAST® or ClustalW. Note that the corresponding positions in a sequence of interest should be determined by comparison with a like for like reference sequence. Should it be desired to determine the corresponding positions in a BIO3-BIO1 enzyme lacking a targeting peptide, then the reference BIO3-BIO1 sequence should also lack a targeting peptide. Suitably in such embodiments the reference sequence used herein may be SEQ ID NO: 294 for BIO3-BIO1 enzymes. Any amino acid positions listed herein in relation to a BIO3-BIO1 enzyme sequences comprising a targeting peptide still apply to a BIO3-BIO1 enzyme sequence from the same organism without a targeting peptide.

A “corresponding” amino acid position to a given SEQ ID NO can be determined using Geneious as a global alignment with free end gaps having the following parameters: cost matrix Blossum 62, gap open penalty 12, gap extension penalty 3, refinement iterations 2; or an equivalent program thereof or an equivalent program thereof.

Unless otherwise stated, a “corresponding” amino acid position to a given SEQ ID NO is determined using EMBOSS Needle default parameters: BLOSUM62; Gap Open 10, GAP EXTEND 0.5; END GAP OPEN 10 and END GAP EXTEND 0.5, or an equivalent program thereof. See, Madeira et al. (2022) Nucleic Acids Research, 1 Jul. 2022, 50(W1):W276-W279; PMID: 35412617 PMCID: PMC9252731. The term “equivalent program” as used herein refers to any sequence comparison program that, for any two sequences in question, generates an alignment having identical corresponding nucleotide or amino acid residue matches when compared to the corresponding alignment generated by the program provided above.

Mutations may include deletions or substitutions or combinations thereof. For example, the mutations may be conservative or non-conservative amino acid substitutions.

“Conservative amino acid substitutions” refer to the interchangeability of residues having similar side chains, and thus typically involves substitution of an amino acid in a polypeptide with amino acids within the same or similar defined class of amino acids. By way of example, an amino acid with an aliphatic side chain may be substituted with another aliphatic amino acid, e.g., alanine, valine, leucine, and isoleucine; an amino acid with hydroxyl side chain may be substituted with another amino acid with a hydroxyl side chain, e.g., serine and threonine; an amino acids having aromatic side chains may be substituted with another amino acid having an aromatic side chain, e.g., phenylalanine, tyrosine, tryptophan, and histidine; an amino acid with a basic side chain may be substituted with another amino acid with a basic side chain, e.g., lysine and arginine; an amino acid with an acidic side chain may be substituted with another amino acid with an acidic side chain, e.g., aspartic acid or glutamic acid; and a hydrophobic or hydrophilic amino acid may be substituted with another hydrophobic or hydrophilic amino acid, respectively. Exemplary conservative substitutions are provided below:

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C | None |
| P | None |

“Non-conservative substitution” refers to substitution of an amino acid in a polypeptide with an amino acid with significantly differing side chain properties. Non-conservative substitutions may use amino acids between, rather than within, the defined groups and may affect (a) the structure of the peptide backbone in the area of the substitution (e.g., proline for glycine) (b) the charge or hydrophobicity, or (c) the bulk of the side chain. By way of example, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification of a polypeptide by removal of one or more amino acids in comparison to a wild-type or control polypeptide. Deletions can comprise removal of 1 or more amino acids, 2 or more amino acids, or 3, 4, 5, 6, 7, 8, 9, 10 or more amino acids of the polypeptide while retaining enzymatic activity. Deletions can comprise a continuous segment or can be discontinuous.

The modified BIO3-BIO1 enzyme may comprise one or more mutations at positions selected from: P347, F348, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, R797 of SEQ ID NO:1 (WT Arabidopsis thaliana sequence), or at corresponding positions thereto. Suitably, the corresponding positions thereto may be those in any homologous sequence to that of SEQ ID NO:1, such as those in SEQ ID NOs 2-14, 271-276 and 319, for example as shown in Table 1.

In one embodiment, modified BIO3-BIO1 enzyme may comprise one or more mutations at positions selected from: C388, A507, F348, G700, P421, P508, R756, S509, W391, and/or W392 of SEQ ID NO:1 (WT Arabidopsis thaliana sequence), or at corresponding positions thereto. Suitably, the corresponding positions thereto may be those in any homologous sequence to that of SEQ ID NO:1, such as those in SEQ ID NOs 2-14, 271-276 and 319, for example as shown in Table 1.

Suitably the modified BIO3-BIO1 enzyme may comprise one or more mutations at positions selected from any of those described in Table 1 below:

TABLE 1
corresponding positions for mutation in homologous BIO3-BIO1 enzymes

| Arabidopsis | Selaginella | | Nannochloropsis | Setaria | Helianthus | Phoenix |
|---|---|---|---|---|---|---|
| thaliana | moellendorffii | Zea mays | gaditana | italica | annuus | dactylifera |
| BIO3-BIO1 | BIO3-BIO1 | BIO3-BIO1 | BIO3-BIO1 | BIO3-BIO1 | BIO3-BIO1 | BIO3-BIO1 |
| (SEQ ID NO: 1) | (SEQ ID NO: 319) | (SEQ ID NO: 2) | (SEQ ID NO: 3) | (SEQ ID NO: 7) | (SEQ ID NO: 10) | (SEQ ID NO: 8) |
| P347 | P357 | P332 | P290 | P333 | P343 | P365 |
| F348 | F358 | F333 | F291 | F334 | F344 | F366 |
| Q350 | Q360 | Q335 | Q293 | Q336 | Q346 | Q368 |
| V354 | V364 | V339 | L297 | V340 | V350 | V372 |
| F370 | F380 | F355 | Y313 | F356 | F366 | F388 |
| C388 | C397 | C372 | C339 | C375 | C384 | C407 |
| A389 | A398 | A373 | A340 | A376 | A385 | A408 |
| S390 | S399 | S374 | S341 | S377 | S386 | S409 |
| W391 | W400 | W375 | W342 | W378 | W387 | W410 |
| W392 | W401 | W376 | W343 | W379 | W388 | W411 |
| T393 | T402 | T377 | T344 | T380 | T389 | T412 |
| M419 | M428 | M403 | I369 | M406 | M415 | M438 |
| F420 | F429 | F404 | F370 | F407 | F416 | F439 |
| P421 | P430 | P405 | P371 | P408 | P417 | P440 |
| Q506 | Q514 | Q494 | A467 | Q497 | Q506 | Q529 |
| A507 | A515 | A495 | A468 | A498 | A507 | A530 |
| P508 | P516 | P496 | P469 | P499 | P508 | P531 |
| S509 | S517 | S497 | T470 | S500 | S509 | S532 |
| P510 | P518 | A498 | I471 | A501 | P510 | S533 |
| Y511 | Y519 | Y499 | F472 | Y502 | Y511 | Y534 |
| T512 | T520 | T500 | T474 | T503 | T512 | T535 |
| G513 | S521 | S501 | G475 | S504 | G513 | G536 |
| Q516 | Q524 | Q504 | Q476 | Q507 | Q516 | Q539 |
| Q517 | Q525 | Q505 | H477 | Q508 | Q517 | Q540 |
| Y520 | Y528 | Y508 | Y480 | Y511 | Y520 | Y543 |
| P529 | P537 | P517 | V489 | P520 | P529 | P552 |
| G608 | A615 | G595 | G600 | G601 | G610 | A633 |
| A609 | A616 | A596 | A601 | A602 | A611 | A634 |
| G610 | G617 | G597 | A602 | G603 | G612 | G635 |
| M612 | M619 | M599 | M604 | M605 | M614 | M637 |
| G700 | G707 | G687 | G692 | G693 | G702 | G725 |
| S704 | T711 | T691 | T696 | T697 | S706 | S729 |
| R756 | R763 | R743 | R765 | R749 | R758 | R781 |
| L786 | V794 | V773 | V797 | V779 | L788 | V811 |
| R790 | R798 | R777 | R801 | R783 | R792 | R815 |
| R797 | R806 | R785 | R808 | R791 | R799 | R822 |

| Quercus | Taxus | Ostreococcus | Physcomitrium | Adiantum | Schizosaccharomyces |
|---|---|---|---|---|---|
| robur | chinensis | tauri | patens | nelumboides | japonicus |
| BIO3-BIO1 | BIO3-BIO1 | BIO3-BIO1 | BIO3-BIO1 | BIO3-BIO1 | BIO3-BIO1 |
| (SEQ ID NO: 11) | (SEQ ID NO: 4) | (SEQ ID NO: 9) | (SEQ ID NO: 5) | (SEQ ID NO: 6) | (SEQ ID NO: 13) |
| P359 | P373 | P331 | P442 | P323 | P262 |
| F360 | F374 | F332 | F443 | F324 | F263 |
| Q362 | Q376 | Q334 | Q445 | Q326 | Q265 |
| V366 | V380 | V338 | V449 | A330 | V269 |
| F382 | F396 | F354 | F465 | F347 | Y285 |
| C400 | C418 | A374 | C486 | S373 | S324 |
| A401 | A419 | A375 | A487 | A374 | A325 |
| S402 | S420 | S376 | S488 | S375 | S326 |
| W403 | W421 | W377 | W489 | W376 | W327 |
| W404 | W422 | W378 | W490 | W377 | W328 |
| T405 | T423 | T379 | T491 | W378 | T329 |
| M431 | M449 | M405 | M517 | L403 | L354 |
| F432 | Y450 | F406 | F518 | F404 | L355 |
| P433 | P451 | P407 | P519 | P405 | P356 |
| Q522 | A533 | Q503 | Q605 | C515 | C433 |
| A523 | F534 | S504 | A606 | E516 | P434 |
| P524 | F535 | P505 | P607 | G517 | P435 |
| S525 | I536 | S506 | S608 | N518 | N436 |
| S526 | E537 | V507 | A609 | I519 | V437 |
| Y527 | M538 | F508 | Y610 | F520 | Y438 |
| T528 | T539 | T509 | T611 | N521 | N439 |
| G529 | M540 | G510 | G612 | — | — |
| Q532 | S544 | Q513 | Q615 | K523 | E442 |
| Q533 | E545 | T514 | Q616 | V524 | V443 |
| Y536 | Y548 | Y517 | H619 | Y527 | Y445 |
| P545 | P557 | P526 | P628 | P536 | S454 |
| G626 | A589 | G621 | G708 | G622 | G525 |
| A627 | S590 | A622 | A709 | A623 | A526 |
| G628 | G591 | G623 | G710 | G624 | G527 |
| M630 | M593 | M625 | M712 | M626 | M529 |
| G718 | G681 | G713 | G800 | G744 | G633 |
| S722 | S685 | T717 | T804 | T748 | T637 |
| R774 | R737 | R777 | R856 | S819 | S708 |
| L804 | V767 | V806 | V886 | L849 | L736 |
| R808 | R771 | R810 | R890 | R853 | R740 |
| R815 | R778 | R817 | R897 | R871 | R754 |

| Gibberella | Thraustotheca | Hordeum | Brassica | Gossypium |
|---|---|---|---|---|
| zeae | clavata | vulgare | napus | hirsutum |
| BIO3-BIO1 | BIO3-BIO1 | BIO3-BIO1 | BIO3-BIO1 | BIO3-BIO1 |
| (SEQ ID NO: 14) | (SEQ ID NO: 12) | (SEQ ID NO. 271) | (SEQ ID NO. 272) | (SEQ ID NO. 273) |
| P269 | P303 | P329 | P347 | P355 |
| F270 | F304 | F330 | F348 | F356 |
| Q272 | Q306 | Q332 | Q350 | Q358 |
| V276 | — | V336 | V354 | V362 |
| F292 | F324 | F352 | F370 | F378 |
| S310 | C341 | C371 | C388 | C396 |
| A311 | A342 | A372 | A389 | A397 |
| S312 | S343 | S373 | S390 | S398 |
| W313 | W344 | W374 | W391 | W399 |
| W314 | W345 | W375 | W392 | W400 |
| S315 | T346 | T376 | T393 | T401 |
| M340 | M371 | M402 | M419 | M427 |
| F341 | F372 | F403 | F420 | F428 |
| A342 | P373 | P404 | P421 | P429 |
| A421 | A456 | Q494 | Q510 | Q518 |
| E422 | E457 | A495 | A511 | A519 |
| P423 | P458 | P496 | P512 | P520 |
| C424 | S459 | S497 | S513 | S521 |
| V425 | V460 | A498 | P514 | S522 |
| Y426 | Y461 | Y499 | Y515 | F523 |
| N427 | N462 | T500 | T516 | T524 |
| — | — | S501 | G517 | G525 |
| K429 | Q465 | Q504 | Q520 | Q528 |
| I430 | H466 | Q505 | Q521 | Q529 |
| Y433 | Y469 | Y508 | Y524 | Y532 |
| Y442 | P478 | P517 | P533 | P541 |
| G516 | G552 | G598 | G613 | G623 |
| A517 | A553 | A599 | A614 | A624 |
| G518 | G554 | G600 | G615 | G625 |
| M520 | M556 | M602 | M617 | M627 |
| G626 | G644 | G690 | G705 | G715 |
| T630 | T648 | T694 | S709 | S719 |
| G689 | R693 | R746 | R761 | R774 |
| Q718 | L721 | I776 | L791 | V804 |
| R722 | R725 | R780 | R795 | R808 |
| R733 | R732 | R788 | R802 | R815 |

| Oryza | Glycine | Triticum |
|---|---|---|
| sativa | max | aestivum |
| BIO3-BIO1 | BIO3-BIO1 | BIO3-BIO1 |
| (SEQ ID NO. 274) | (SEQ ID NO. 275) | (SEQ ID NO. 276) |
| P331 | P330 | P329 |
| F332 | F331 | F330 |
| Q334 | Q333 | Q332 |
| V338 | V337 | V336 |
| F354 | F353 | F352 |
| C371 | C371 | C371 |
| A372 | A372 | A372 |
| S373 | S373 | S373 |
| W374 | W374 | W374 |
| W375 | W375 | W375 |
| T376 | T376 | T376 |
| M402 | M402 | M402 |
| F403 | F403 | F403 |
| P404 | P404 | P404 |
| Q493 | Q493 | Q493 |
| A494 | A594 | A494 |
| P495 | P595 | P495 |
| S496 | S496 | S496 |
| A497 | S497 | A497 |
| Y498 | Y498 | Y498 |
| T499 | T499 | T499 |
| S500 | G500 | S500 |
| Q503 | Q503 | Q503 |
| Q504 | Q504 | Q504 |
| Y507 | Y507 | Y507 |
| P516 | P516 | P516 |
| G597 | G594 | G597 |
| A598 | A595 | A598 |
| G599 | G596 | G599 |
| M601 | M598 | M601 |
| G689 | G686 | G689 |
| T693 | S690 | T693 |
| R745 | R742 | R745 |
| I775 | L772 | I775 |
| R779 | R776 | R779 |
| R787 | R783 | R787 |

For example, the modified BIO3-BIO1 enzyme may comprise one or more mutations at positions selected from: P332, F333, Q335, V339, F355, C372, A373, S374, W375, W376, T377, M403, F404, P405, Q494, A495, P496, T497, A498, Y499, T500, G501, Q504, Q505, Y508, P517, G595, A596, G597, M599, G687, T691, R743, V773, R777, and R785 of SEQ ID NO:2 (WT Zea mays sequence).

For example, the modified BIO3-BIO1 enzyme may comprise one or more mutations at positions selected from: P290, F291, Q293, L297, Y313, 0339, A340, S341, W342, W343, T344, 1369, F370, P371, A467, A468, P469, T470, 1471, F472, T474, G475, Q476, H477, Y480, V489, G600, A601, A602, M604, G692, T696, R765, V797, R801, and R808 of SEQ ID NO:3 (WT Nannochloropsis gaditana sequence).

For example, the modified BIO3-BIO1 enzyme may comprise one or more mutations at positions selected from: P331, F332, Q334, V338, F354, A374, A375, S376, W377, W378, T379, M405, F406, P407, Q503, P504, P505, N506, V507, F508, T509, G510, Q513, T514, Y517, P526, G621, A622, G623, M625, G713, T717, R777, V806, R810, and R817. Of SEQ ID NO:9 (WT Ostreococcus tauri sequence).

For example, the modified BIO3-BIO1 enzyme may comprise one or more mutations at positions selected from: P262, F263, Q265, V269, Y285, S324, A325, S326, W327, W328, T329, L354, L355, P356, 0433, P434, P435, N436, V437, Y438, N439, E442, V443, Y445, S454, G525, A526, G527, M529, G633, T637, S708, L736, R740, and R754. of SEQ ID NO:13 (WT Schizosaccharomyces japonicus sequence).

For example, the modified BIO3-BIO1 enzyme may comprise one or more mutations at positions selected from: P331, F332, Q334, V338, F354, C371, A372, S373, W374, W375, T376, M402, F403, P404, Q493, A494, P495, S496, A497, Y498, T499, S500, Q503, Q504, Y507, P516, G597, A598, G599, M601, G689, T693, R745, 1775, R779, and R787 of SEQ ID NO:274 (WT Oryza sativa sequence).

For example, the modified BIO3-BIO1 enzyme may comprise one or more mutations at positions selected from: P343, F344, Q346, V350, F366, C384, A385, S386, W387, W388, T389, M415, F416, P417, Q506, A507, P508, S509, P510, Y511, T512, G513, Q516, Q517, Y520, P529, G610, A611, G612, M614, G702, S706, R758, L788, R792, and R799 of SEQ ID NO:10 (WT Helianthus annuus sequence).

For example, the modified BIO3-BIO1 enzyme may comprise one or more mutations at positions selected from: P333, F334, Q336, V340, F356, C375, A376, S377, W378, W379, T380, M406, F407, P408, Q497, A498, P499, S500, A501, Y502, T503, S504, Q507, Q508, Y511, P520, G601, A602, G603, M605, G693, T697, R749, V779, R783, and R791 of SEQ ID NO:10 (WT Setaria italica sequence).

For example, the modified BIO3-BIO1 enzyme may comprise one or more mutations at positions selected from: P357, F358, Q360, V364, F380, C397, A398, S399, W400, W401, T402, M428, F429, P430, Q514, A515, P516, S517, P518, Y519, T520, S521, Q524, Q525, Y528, P537, A615, A616, G617, M619, G707, T711, R763, V794, R798, and R806 of SEQ ID NO:319 (WT Selaginella moellendorffii sequence).

In one embodiment, the modified BIO3-BIO1 enzyme is derived from Ostreococcus tauri and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO:9 or 300 and comprises the mutation A374D. In one embodiment, the BIO3-BIO1 enzyme is derived from Ostreococcus tauri and comprises an amino acid sequence according to SEQ ID NO:321. In one embodiment, the BIO3-BIO1 enzyme is derived from Ostreococcus tauri and consists of an amino acid sequence according to SEQ ID NO:321.

In one embodiment, the modified BIO3-BIO1 enzyme is derived from Ostreococcus tauri and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO: 9 or 300 and comprises the mutation F332D. In one embodiment, the BIO3-BIO1 enzyme is derived from Ostreococcus tauri and comprises an amino acid sequence according to SEQ ID NO:322. In one embodiment, the BIO3-BIO1 enzyme is derived from Ostreococcus tauri and consists of an amino acid sequence according to SEQ ID NO:322.

In one embodiment, the modified BIO3-BIO1 enzyme is derived from Ostreococcus tauri and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO: 9 or 300 and comprises the mutation P407A. In one embodiment, the BIO3-BIO1 enzyme is derived from Ostreococcus tauri and comprises an amino acid sequence according to SEQ ID NO:323. In one embodiment, the BIO3-BIO1 enzyme is derived from Ostreococcus tauri and consists of an amino acid sequence according to SEQ ID NO:323.

In one embodiment, the modified BIO3-BIO1 enzyme is derived from Zea mays and comprises an amino acid sequence having at least 35%, at least 40%, at least 45%, at least 50%, at least 55%, at least 60%, at least 65%, at least 70%, at least 75%, least 80%, at least 90%, at least 95%, at least 96%, at least 97%, at least 98%, at least 99% identity according to SEQ ID NO: 2 or 295 and comprises the mutation C372D. In one embodiment, the BIO3-BIO1 enzyme is derived from Zea mays and comprises an amino acid sequence according to SEQ ID NO:324. In one embodiment, the BIO3-BIO1 enzyme is derived from Zea mays and consists of an amino acid sequence according to SEQ ID NO:324.

In one embodiment the modified BIO3-BIO1 enzyme may have at least 30% identity to an amino acid sequence according to any of SEQ ID Numbers 1-14, 271-276 and 319, (or SEQ ID NO: 294 to 310 and 320), or a functional fragment thereof, wherein the amino acid sequence or fragment comprises one or more mutations at positions selected from: P347, F348, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, R797 defined in relation to SEQ ID NO:1, or at corresponding positions thereto. Suitably, the corresponding positions thereto may be those in any homologous sequence to that of SEQ ID NO:1, such as those in SEQ ID NOs 2-14, 271-276 and 319, for example as shown in Table 1.

In one embodiment the modified BIO3-BIO1 enzyme may have at least 30% identity to an amino acid sequence according to any of SEQ ID Numbers 1-14, 271-276 and 319, (or SEQ ID NO: 294 to 310 and 320), or a functional fragment thereof, wherein the amino acid sequence or fragment comprises one or more mutations at positions selected from: C388, A507, F348, G700, P421, P508, R756, S509, W391, and/or W392 defined in relation to SEQ ID NO:1, or at corresponding positions thereto. Suitably, the corresponding positions thereto may be those in any homologous sequence to that of SEQ ID NO:1, such as those in SEQ ID NOs 2-14, 271-276 and 319, for example as shown in Table 1.

In one embodiment the modified BIO3-BIO1 enzyme may have at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID Numbers 1-14, 271-276 and 319, (or SEQ ID NO: 294 to 310 and 320), or a functional fragment thereof, wherein the amino acid sequence or fragment comprises one or more mutations at positions selected from: P347, F348, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, R797 defined in relation to SEQ ID NO:1, or at corresponding positions thereto. Suitably, the corresponding positions thereto may be those in any homologous sequence to that of SEQ ID NO:1, such as those in SEQ ID NOs 2-14, 271-276 and 319, for example as shown in Table 1.

In one embodiment the modified BIO3-BIO1 enzyme may have at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID Numbers 1-14, 271-276 and 319, (or SEQ ID NO: 294 to 310 and 320), or a functional fragment thereof, wherein the amino acid sequence or fragment comprises one or more mutations at positions selected from: C388, A507, F348, G700, P421, P508, R756, S509, W391, and/or W392 defined in relation to SEQ ID NO:1, or at corresponding positions thereto. Suitably, the corresponding positions thereto may be those in any homologous sequence to that of SEQ ID NO:1, such as those in SEQ ID NOs 2-14, 271-276 and 319, for example as shown in Table 1.

The modified BIO3-BIO1 enzyme may comprise an amino acid sequence that has a mutation at position P347 of SEQ ID NO:1 (WT Arabidopsis thaliana sequence) or at a corresponding position thereto, such as position P332 of SEQ ID NO:2 (Zea mays sequence), position P331 of SEQ ID NO:274 (Oryza sativa sequence), position P343 of SEQ ID NO:10 (Helianthus annuus sequence), position P333 of SEQ ID NO:7 (Setaria italica sequence), position P290 of SEQ ID NO:3 (Nannochloropsis gaditana sequence), position P331 of SEQ ID NO:9 (Ostreococcus tauri sequence), or position P262 of SEQ ID NO:13 (Schizosaccharomyces japonicus sequence). The mutation may be a substitution of P. The substitution may be a non-conservative mutation. P may be substituted with an A or E residue. In such an embodiment, the BIO3-BIO1 enzyme may comprise or consist of a sequence according to SEQ ID NO: 100 or 101, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position F348 of SEQ ID NO:1 (WT Arabidopsis thaliana sequence) or at a corresponding position thereto, such as position F333 of SEQ ID NO:2 (Zea mays sequence), position F332 of SEQ ID NO:274 (Oryza sativa sequence), position F344 of SEQ ID NO:10 (Helianthus annuus sequence), position F334 of SEQ ID NO:7 (Setaria italica sequence),position F291 of SEQ ID NO:3 (Nannochloropsis gaditana sequence), position F332 of SEQ ID NO:9 (Ostreococcus tauri sequence), or position F263 of SEQ ID NO:13 (Schizosaccharomyces japonicus sequence). The mutation may be a substitution of F. The substitution may be a non-conservative mutation. F may be substituted with an A/C/D/E/I/K/M/N/Q/S/T/V residue. In such an embodiment, the BIO3-BIO1 enzyme may comprise or consist of a sequence according to SEQ ID NO: 28-33, 84-89, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position Q350 of SEQ ID NO:1 (WT Arabidopsis thaliana sequence) or at a corresponding position thereto, such as position Q335 of SEQ ID NO:2 (Zea mays sequence), position Q334 of SEQ ID NO:274 (Oryza sativa sequence), position Q346 of SEQ ID NO:10 (Helianthus annuus sequence), position Q336 of SEQ ID NO:7 (Setaria italica sequence), position Q293 of SEQ ID NO:3 (Nannochloropsis gaditana sequence), position Q334 of SEQ ID NO:9 (Ostreococcus tauri sequence) or position Q265 of SEQ ID NO:13 (Schizosaccharomyces japonicus sequence). The mutation may be a substitution of Q. The substitution may be a non-conservative mutation. Q may be substituted with an H or S residue. In such an embodiment, the BIO3-BIO1 enzyme may comprise or consist of a sequence according to SEQ ID NO: 112-113, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position V354 of SEQ ID NO:1 (WT Arabidopsis thaliana sequence) or at a corresponding position thereto, such as position V339 of SEQ ID NO:2 (Zea mays sequence), position V338 of SEQ ID NO:274 (Oryza sativa sequence), position V350 of SEQ ID NO:10 (Helianthus annuus sequence), position V340 of SEQ ID NO:7 (Setaria italica sequence), position L297 of SEQ ID NO:3 (Nannochloropsis gaditana sequence), position V338 of SEQ ID NO:9 (Ostreococcus tauri sequence) or position V269 of SEQ ID NO:13 (Schizosaccharomyces japonicus sequence). The mutation may be a substitution of V. The substitution may be a non-conservative mutation. V may be substituted with an A/E/L/N/T residue. In such an embodiment, the BIO3-BIO1 enzyme may comprise or consist of a sequence according to SEQ ID NO: 140-144, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position F370 of SEQ ID NO:1 (WT Arabidopsis thaliana sequence) or at a corresponding position thereto, such as position F355 of SEQ ID NO:2 (Zea mays sequence), position F354 of SEQ ID NO:274 (Oryza sativa sequence), position F366 of SEQ ID NO:10 (Helianthus annuus sequence), position F356 of SEQ ID NO:7 (Setaria italica sequence), position Y313 of SEQ ID NO:3 (Nannochloropsis gaditana sequence), position F354 of SEQ ID NO:9 (Ostreococcus tauri sequence) or position Y285 of SEQ ID NO:13 (Schizosaccharomyces japonicus sequence). The mutation may be a substitution of F. The substitution may be a non-conservative mutation. F may be substituted with an L residue. In such an embodiment, the BIO3-BIO1 enzyme may comprise or consist of a sequence according to SEQ ID NO: 90, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position C388 of SEQ ID NO:1 (WT Arabidopsis thaliana sequence) or at a corresponding position thereto, such as position C372 of SEQ ID NO:2 (Zea mays sequence), position C371 of SEQ ID NO:274 (Oryza sativa sequence), position C384 of SEQ ID NO:10 (Helianthus annuus sequence), position C375 of SEQ ID NO:7 (Setaria italica sequence), position C339 of SEQ ID NO:3 (Nannochloropsis gaditana sequence), position A374 of SEQ ID NO:9 (Ostreococcus tauri sequence) or position S324 of SEQ ID NO:13 (Schizosaccharomyces japonicus sequence). The mutation may be a substitution of C. The substitution may be a non-conservative mutation. C may be substituted with an D/M/T residue. In such an embodiment, the BIO3-BIO1 enzyme may comprise or consist of a sequence according to SEQ ID NO: 26-27, 83, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position A389 of SEQ ID NO:1 (WT Arabidopsis thaliana sequence) or at a corresponding position thereto, such as position A373 of SEQ ID NO:2 (Zea mays sequence), position A372 of SEQ ID NO:274 (Oryza sativa sequence), position A385 of SEQ ID NO:10 (Helianthus annuus sequence), position A376 of SEQ ID NO:7 (Setaria italica sequence), position A340 of SEQ ID NO:3 (Nannochloropsis gaditana sequence), position A375 of SEQ ID NO:9 (Ostreococcus tauri sequence) or position A325 of SEQ ID NO:13 (Schizosaccharomyces japonicus sequence). The mutation may be a substitution of A. The substitution may be a non-conservative mutation. A may be substituted with an F residue. In such an embodiment, the BIO3-BIO1 enzyme may comprise or consist of a sequence according to SEQ ID NO: 79, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position S390 of SEQ ID NO:1 (WT Arabidopsis thaliana sequence), at a corresponding position thereto, such as position S374 of SEQ ID NO:2 (Zea mays sequence), position S373 of SEQ ID NO:274 (Oryza sativa sequence), position S386 of SEQ ID NO:10 (Helianthus annuus sequence), position S377 of SEQ ID NO:7 (Setaria italica sequence), position S341 of SEQ ID NO:3 (Nannochloropsis gaditana sequence), position S376 of SEQ ID NO:9 (Ostreococcus tauri sequence) or position S326 of SEQ ID NO:13 (Schizosaccharomyces japonicus sequence). The mutation may be a substitution of S. The substitution may be a non-conservative mutation. S may be substituted with a C residue. In such an embodiment, the BIO3-BIO1 enzyme may comprise or consist of a sequence according to SEQ ID NO: 120, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position W391 of SEQ ID NO:1 (WT Arabidopsis thaliana sequence) or at a corresponding position thereto, such as position W375 of SEQ ID NO:2 (Zea mays sequence), position W374 of SEQ ID NO:274 (Oryza sativa sequence), position W387 of SEQ ID NO:10 (Helianthus annuus sequence), position W378 of SEQ ID NO:7 (Setaria italica sequence), position W342 of SEQ ID NO:3 (Nannochloropsis gaditana sequence), position W377 of SEQ ID NO:9 (Ostreococcus tauri sequence) or position W327 of SEQ ID NO:13 (Schizosaccharomyces japonicus sequence). The mutation may be a substitution of W. The substitution may be a non-conservative mutation. W may be substituted with an F/L/M residue. In such an embodiment, the BIO3-BIO1 enzyme may comprise or consist of a sequence according to SEQ ID NO: 66-67, 145, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position W392 of SEQ ID NO:1 (WT Arabidopsis thaliana sequence) or at a corresponding position thereto, such as position W376 of SEQ ID NO:2 (Zea mays sequence), position W375 of SEQ ID NO:274 (Oryza sativa sequence), position W388 of SEQ ID NO:10 (Helianthus annuus sequence), position W379 of SEQ ID NO:7 (Setaria italica sequence), position W343 of SEQ ID NO:3 (Nannochloropsis gaditana sequence), position W378 of SEQ ID NO:9 (Ostreococcus tauri sequence) or position W328 of SEQ ID NO:13 (Schizosaccharomyces japonicus sequence). The mutation may be a substitution of W. The substitution may be a non-conservative mutation. W may be substituted with an A/C/D/G/M/S residue. In such an embodiment, the BIO3-BIO1 enzyme may comprise or consist of a sequence according to SEQ ID NO: 68-70, 146-148, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position T393 of SEQ ID NO:1 (WT Arabidopsis thaliana sequence), or at a corresponding position thereto, such as position T377 of SEQ ID NO:2 (Zea mays sequence), position T376 of SEQ ID NO:274 (Oryza sativa sequence), position T389 of SEQ ID NO:10 (Helianthus annuus sequence), position T380 of SEQ ID NO:7 (Setaria italica sequence), position T344 of SEQ ID NO:3 (Nannochloropsis gaditana sequence), position T379 of SEQ ID NO:9 (Ostreococcus tauri sequence) or position T329 of SEQ ID NO:13 (Schizosaccharomyces japonicus sequence). The mutation may be a substitution of T. The substitution may be a non-conservative mutation. T may be substituted with a V residue. In such an embodiment, the BIO3-BIO1 enzyme may comprise or consist of a sequence according to SEQ ID NO: 135, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position M419 of SEQ ID NO:1 (WT Arabidopsis thaliana sequence) or at a corresponding position thereto, such as position M403 of SEQ ID NO:2 (Zea mays sequence), position M402 of SEQ ID NO:274 (Oryza sativa sequence), position M415 of SEQ ID NO:10 (Helianthus annuus sequence), position M406 of SEQ ID NO:7 (Setaria italica sequence), position 1369 of SEQ ID NO:3 (Nannochloropsis gaditana sequence), position M405 of SEQ ID NO:9 (Ostreococcus tauri sequence) or position L354 of SEQ ID NO:13 (Schizosaccharomyces japonicus sequence). The mutation may be a substitution of M. The substitution may be a non-conservative mutation. M may be substituted with an I residue. In such an embodiment, the BIO3-BIO1 enzyme may comprise or consist of a sequence according to SEQ ID NO: 98, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position F420 of SEQ ID NO:1 (WT Arabidopsis thaliana sequence), or at a corresponding position thereto, such as position F404 of SEQ ID NO:2 (Zea mays sequence), position F403 of SEQ ID NO:274 (Oryza sativa sequence), position F416 of SEQ ID NO:10 (Helianthus annuus sequence), position F407 of SEQ ID NO:7 (Setaria italica sequence), position F370 of SEQ ID NO:3 (Nannochloropsis gaditana sequence), position F406 of SEQ ID NO:9 (Ostreococcus tauri sequence) or position L355 of SEQ ID NO:13 (Schizosaccharomyces japonicus sequence). The mutation may be a substitution of F. The substitution may be a non-conservative mutation. F may be substituted with an I residue. In such an embodiment, the BIO3-BIO1 enzyme may comprise or consist of a sequence according to SEQ ID NO: 91, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position P421 of SEQ ID NO:1 (WT Arabidopsis thaliana sequence) or at a corresponding position thereto, such as position P405 of SEQ ID NO:2 (Zea mays sequence), position P404 of SEQ ID NO:274 (Oryza sativa sequence), position P417 of SEQ ID NO:10 (Helianthus annuus sequence), position P408 of SEQ ID NO:7 (Setaria italica sequence), position P371 of SEQ ID NO:3 (Nannochloropsis gaditanaa sequence), position P407 of SEQ ID NO:9 (Ostreococcus tauri sequence) or position P356 of SEQ ID NO:13 (Schizosaccharomyces japonicus sequence). The mutation may be a substitution of P. The substitution may be a non-conservative mutation. P may be substituted with an A/E/G/L/W residue. In such an embodiment, the BIO3-BIO1 enzyme may comprise or consist of a sequence according to SEQ ID NO: 41, 102-105, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position Q506 of SEQ ID NO:1 (WT Arabidopsis thaliana sequence) or at a corresponding position thereto, such as position Q494 of SEQ ID NO:2 (Zea mays sequence), position Q493 of SEQ ID NO:274 (Oryza sativa sequence), position Q506 of SEQ ID NO:10 (Helianthus annuus sequence), position Q497 of SEQ ID NO:7 (Setaria italica sequence), position A467 of SEQ ID NO:3 (Nannochloropsis gaditana sequence), position Q503 of SEQ ID NO:9 (Ostreococcus tauri sequence) or position P407 of SEQ ID NO:9 (Ostreococcus tauri sequence) or position C433 of SEQ ID NO:13 (Schizosaccharomyces japonicus sequence). The mutation may be a substitution of Q. The substitution may be a non-conservative mutation. Q may be substituted with an A residue. In such an embodiment, the BIO3-BIO1 enzyme may comprise or consist of a sequence according to SEQ ID NO: 46, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position A507 of SEQ ID NO:1 (WT Arabidopsis thaliana sequence) or at a corresponding position thereto, such as position A495 of SEQ ID NO:2 (Zea mays sequence), position A494 of SEQ ID NO:274 (Oryza sativa sequence), position A507 of SEQ ID NO:10 (Helianthus annuus sequence), position A498 of SEQ ID NO:7 (Setaria italica sequence), position A468 of SEQ ID NO:3 (Nannochloropsis gaditana sequence), position S504 of SEQ ID NO:9 (Ostreococcus tauri sequence) or position P434 of SEQ ID NO:13 (Schizosaccharomyces japonicus sequence). The mutation may be a substitution of A. The substitution may be a non-conservative mutation. A may be substituted with a K or S residue. In such an embodiment, the BIO3-BIO1 enzyme may comprise or consist of a sequence according to SEQ ID NO: 15-16, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position P508 of SEQ ID NO:1 (WT Arabidopsis thaliana sequence) or at a corresponding position thereto, such as position P496 of SEQ ID NO:2 (Zea mays sequence), position P495 of SEQ ID NO:274 (Oryza sativa sequence), position P508 of SEQ ID NO:10 (Helianthus annuus sequence), position P499 of SEQ ID NO:7 (Setaria italica sequence), position P499 of SEQ ID NO:3 (Nannochloropsis gaditana sequence), position P505 of SEQ ID NO:9 (Ostreococcus tauri sequence) or position P435 of SEQ ID NO:13 (Schizosaccharomyces japonicus sequence). The mutation may be a substitution of P or a deletion of P. The substitution may be a non-conservative mutation. P may be substituted with an L or T residue. In such an embodiment, the BIO3-BIO1 enzyme may comprise or consist of a sequence according to SEQ ID NO: 42-44, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position S509 of SEQ ID NO:1 (WT Arabidopsis thaliana sequence) or at a corresponding position thereto, such as position S497 of SEQ ID NO:2 (Zea mays sequence), position S496 of SEQ ID NO:274 (Oryza sativa sequence), position S509 of SEQ ID NO:10 (Helianthus annuus sequence), position S500 of SEQ ID NO:7 (Setaria italica sequence), position T470 of SEQ ID NO:3 (Nannochloropsis gaditana sequence), position S506 of SEQ ID NO:9 (Ostreococcus tauri sequence) or position N436 of SEQ ID NO:13 (Schizosaccharomyces japonicus sequence). The mutation may be a substitution of S. The substitution may be a non-conservative mutation. S may be substituted with a A/C/D/E/F/G/H/I/K/L/M/N/Q/R/S/T/V/W/Y residue. In such an embodiment, the BIO3-BIO1 enzyme may comprise or consist of a sequence according to SEQ ID NO: 54-59, 122-133, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position P510 of SEQ ID NO:1 (WT Arabidopsis thaliana sequence) or at a corresponding position thereto, such as position A498 of SEQ ID NO:2 (Zea mays sequence), position A497 of SEQ ID NO:274 (Oryza sativa sequence), position P510 of SEQ ID NO:10 (Helianthus annuus sequence), position A501 of SEQ ID NO:7 (Setaria italica sequence), position 1471 of SEQ ID NO:3 (Nannochloropsis gaditana sequence), position V507 of SEQ ID NO:9 (Ostreococcus tauri sequence) or position V437 of SEQ ID NO:13 (Schizosaccharomyces japonicus sequence). The mutation may be a substitution of P. The substitution may be a non-conservative mutation. P may be substituted with a A/C/E/LQ/V residue. In such an embodiment, the BIO3-BIO1 enzyme may comprise or consist of a sequence according to SEQ ID NO: 45, 106-110, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position Y511 of SEQ ID NO:1 (WT Arabidopsis thaliana sequence), at a corresponding position thereto, such as position Y499 of SEQ ID NO:2 (Zea mays sequence), position Y498 of SEQ ID NO:274 (Oryza sativa sequence), position Y511 of SEQ ID NO:10 (Helianthus annuus sequence), position Y502 of SEQ ID NO:7 (Setaria italica sequence), position F472 of SEQ ID NO:3 (Nannochloropsis gaditana sequence), position F508 of SEQ ID NO:9 (Ostreococcus tauri sequence) or position Y438 of SEQ ID NO:13 (Schizosaccharomyces japonicus sequence). The mutation may be a substitution of Y. The substitution may be a non-conservative mutation. Y may be substituted with a C/D/E/F/H/I/K/M/P/Q/R/V/W residue. In such an embodiment, the BIO3-BIO1 enzyme may comprise or consist of a sequence according to SEQ ID NO: 71-78, 149-153, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position T512 of SEQ ID NO:1 (WT Arabidopsis thaliana sequence) or at a corresponding position thereto, such as position T500 of SEQ ID NO:2 (Zea mays sequence), position T499 of SEQ ID NO:274 (Oryza sativa sequence), position T512 of SEQ ID NO:10 (Helianthus annuus sequence), position T503 of SEQ ID NO:7 (Setaria italica sequence), position T474 of SEQ ID NO:3 (Nannochloropsis gaditana sequence), position T509 of SEQ ID NO:9 (Ostreococcus tauri sequence) or position N439 of SEQ ID NO:13 (Schizosaccharomyces japonicus sequence). The mutation may be a substitution of T. The substitution may be a non-conservative mutation. T may be substituted with a C/D/G/I/N/Q/R/V/W residue. In such an embodiment, the BIO3-BIO1 enzyme may comprise or consist of a sequence according to SEQ ID NO: 61-65, 136-139, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position G513 of SEQ ID NO:1 (WT Arabidopsis thaliana sequence) or at a corresponding position thereto, such as position S501 of SEQ ID NO:2 (Zea mays sequence), position S500 of SEQ ID NO:274 (Oryza sativa sequence), position G513 of SEQ ID NO:10 (Helianthus annuus sequence), position S504 of SEQ ID NO:7 (Setaria italica sequence), position G475 of SEQ ID NO:3 (Nannochloropsis gaditana sequence) or position G510 of SEQ ID NO:9 (Ostreococcus tauri sequence). The mutation may be a substitution of G, or a deletion of G. The substitution may be a non-conservative mutation. G may be substituted with a A/LIP residue. In such an embodiment, the BIO3-BIO1 enzyme may comprise or consist of a sequence according to SEQ ID NO: 34-36, 92, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position Q516 of SEQ ID NO:1 (WT Arabidopsis thaliana sequence) or at a corresponding position thereto, such as position Q504 of SEQ ID NO:2 (Zea mays sequence), position Q503 of SEQ ID NO:274 (Oryza sativa sequence), position Q516 of SEQ ID NO:10 (Helianthus annuus sequence), position Q507 of SEQ ID NO:7 (Setaria italica sequence), position Q476 of SEQ ID NO:3 (Nannochloropsis gaditana sequence), position Q513 of SEQ ID NO:9 (Ostreococcus tauri sequence) or position E442 of SEQ ID NO:13 (Schizosaccharomyces japonicus sequence). The mutation may be a substitution of Q. The substitution may be a non-conservative mutation. Q may be substituted with a C residue. In such an embodiment, the BIO3-BIO1 enzyme may comprise or consist of a sequence according to SEQ ID NO: 47, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position Q517 of SEQ ID NO:1 (WT Arabidopsis thaliana sequence) or at a corresponding position thereto, such as position Q505 of SEQ ID NO:2 (Zea mays sequence), position Q504 of SEQ ID NO:274 (Oryza sativa sequence), position Q517 of SEQ ID NO:10 (Helianthus annuus sequence), position Q508 of SEQ ID NO:7 (Setaria italica sequence), position H477 of SEQ ID NO:3 (Nannochloropsis gaditana sequence), position T514 of SEQ ID NO:9 (Ostreococcus tauri sequence) or position V443 of SEQ ID NO:13 (Schizosaccharomyces japonicus sequence). The mutation may be a substitution of Q. The substitution may be a non-conservative mutation. Q may be substituted with a D/F/H/1/M/T/W/Y residue. In such an embodiment, the BIO3-BIO1 enzyme may comprise or consist of a sequence according to SEQ ID NO: 48-52, 114-116, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position Y520 of SEQ ID NO:1 (WT Arabidopsis thaliana sequence), or at a corresponding position thereto, such as position Y508 of SEQ ID NO:2 (Zea mays sequence), position Y507 of SEQ ID NO:274 (Oryza sativa sequence), position Y520 of SEQ ID NO:10 (Helianthus annuus sequence), position Y511 of SEQ ID NO:7 (Setaria italica sequence), position Y480 of SEQ ID NO:3 (Nannochloropsis gaditana sequence), position Y517 of SEQ ID NO:9 (Ostreococcus tauri sequence) or position Y445 of SEQ ID NO:13 (Schizosaccharomyces japonicus sequence). The mutation may be a substitution of Y. The substitution may be a non-conservative mutation. Y may be substituted with a N or W residue. In such an embodiment, the BIO3-BIO1 enzyme may comprise or consist of a sequence according to SEQ ID NO: 154-155, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position P529 of SEQ ID NO:1 (WT Arabidopsis thaliana sequence) or at a corresponding position thereto, such as position P517 of SEQ ID NO:2 (Zea mays sequence), position P516 of SEQ ID NO:274 (Oryza sativa sequence), position P529 of SEQ ID NO:10 (Helianthus annuus sequence), position P520 of SEQ ID NO:7 (Setaria italica sequence), position V489 of SEQ ID NO:3 (Nannochloropsis gaditana sequence), position P526 of SEQ ID NO:9 (Ostreococcus tauri sequence) or position S454 of SEQ ID NO:13 (Schizosaccharomyces japonicus sequence). The mutation may be a substitution of P. The substitution may be a non-conservative mutation. P may be substituted with an A residue. In such an embodiment, the BIO3-BIO1 enzyme may comprise or consist of a sequence according to SEQ ID NO: 111, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position G608 of SEQ ID NO:1 (WT Arabidopsis thaliana sequence) or at a corresponding position thereto, such as position G595 of SEQ ID NO:2 (Zea mays sequence), position G597 of SEQ ID NO:274 (Oryza sativa sequence), position G610 of SEQ ID NO:10 (Helianthus annuus sequence), position G601 of SEQ ID NO:7 (Setaria italica sequence), position G600 of SEQ ID NO:3 (Nannochloropsis gaditana sequence), position G621 of SEQ ID NO:9 (Ostreococcus tauri sequence) or position G525 of SEQ ID NO:13 (Schizosaccharomyces japonicus sequence). The mutation may be a substitution of G. The substitution may be a non-conservative mutation. G may be substituted with an A/E/I residue. In such an embodiment, the BIO3-BIO1 enzyme may comprise or consist of a sequence according to SEQ ID NO: 37, 93-94, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position A609 of SEQ ID NO:1 (WT Arabidopsis thaliana sequence), or at a corresponding position thereto, such as position A596 of SEQ ID NO:2 (Zea mays sequence), position A598 of SEQ ID NO:274 (Oryza sativa sequence), position A611 of SEQ ID NO:10 (Helianthus annuus sequence), position A602 of SEQ ID NO:7 (Setaria italica sequence), position A601 of SEQ ID NO:3 (Nannochloropsis gaditana sequence), position A622 of SEQ ID NO:9 (Ostreococcus tauri sequence) or position A526 of SEQ ID NO:13 (Schizosaccharomyces japonicus sequence). The mutation may be a substitution of A. The substitution may be a non-conservative mutation. A may be substituted with an C/F/H/I/K/M/N/R/T/V/W/Y residue. In such an embodiment, the BIO3-BIO1 enzyme may comprise or consist of a sequence according to SEQ ID NO: 17-25, 80-82, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position G610 of SEQ ID NO:1 (WT Arabidopsis thaliana sequence) or at a corresponding position thereto, such as position G597 of SEQ ID NO:2 (Zea mays sequence), position G599 of SEQ ID NO:274 (Oryza sativa sequence), position G612 of SEQ ID NO:10 (Helianthus annuus sequence), position G603 of SEQ ID NO:7 (Setaria italica sequence), position G623 of SEQ ID NO:9 (Ostreococcus tauri sequence), position A602 of SEQ ID NO:3 (Nannochloropsis gaditana sequence) or position G527 of SEQ ID NO:13 (Schizosaccharomyces japonicus sequence). The mutation may be a substitution of G. The substitution may be a non-conservative mutation. G may be substituted with an H residue. In such an embodiment, the BIO3-BIO1 enzyme may comprise or consist of a sequence according to SEQ ID NO: 95, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position M612 of SEQ ID NO:1 (WT Arabidopsis thaliana sequence) or at a corresponding position thereto, such as position M599 of SEQ ID NO:2 (Zea mays sequence), position M601 of SEQ ID NO:274 (Oryza sativa sequence), position M614 of SEQ ID NO:10 (Helianthus annuus sequence), position M605 of SEQ ID NO:7 (Setaria italica sequence), position M604 of SEQ ID NO:3 (Nannochloropsis gaditana sequence), position M625 of SEQ ID NO:9 (Ostreococcus tauri sequence) or position M529 of SEQ ID NO:13 (Schizosaccharomyces japonicus sequence). The mutation may be a substitution of M. The substitution may be a non-conservative mutation. M may be substituted with an L residue. In such an embodiment, the BIO3-BIO1 enzyme may comprise or consist of a sequence according to SEQ ID NO: 99, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position G700 of SEQ ID NO:1 (WT Arabidopsis thaliana sequence) or at a corresponding position thereto, such as position G687 of SEQ ID NO:2 (Zea mays sequence), position G689 of SEQ ID NO:274 (Oryza sativa sequence), position G702 of SEQ ID NO:10 (Helianthus annuus sequence), position G693 of SEQ ID NO:7 (Setaria italica sequence), position G692 of SEQ ID NO:3 (Nannochloropsis gaditana sequence), position G713 of SEQ ID NO:9 (Ostreococcus tauri sequence) or position G633 of SEQ ID NO:13 (Schizosaccharomyces japonicus sequence). The mutation may be a substitution of G. The substitution may be a non-conservative mutation. G may be substituted with an A/C/S residue. In such an embodiment, the BIO3-BIO1 enzyme may comprise or consist of a sequence according to SEQ ID NO: 38-39, 96, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position S704 of SEQ ID NO:1 (WT Arabidopsis thaliana sequence) or at a corresponding position thereto, such as position T691 of SEQ ID NO:2 (Zea mays sequence), position T693 of SEQ ID NO:274 (Oryza sativa sequence), position S706 of SEQ ID NO:10 (Helianthus annuus sequence), position T697 of SEQ ID NO:7 (Setaria italica sequence), position T696 of SEQ ID NO:3 (Nannochloropsis gaditana sequence), position T717 of SEQ ID NO:9 (Ostreococcus tauri sequence) or position T637 of SEQ ID NO:13 (Schizosaccharomyces japonicus sequence). The mutation may be a substitution of S. The substitution may be a non-conservative mutation. S may be substituted with an H or P residue. In such an embodiment, the BIO3-BIO1 enzyme may comprise or consist of a sequence according to SEQ ID NO: 60, 134, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position R756 of SEQ ID NO:1 (WT Arabidopsis thaliana sequence) or at a corresponding position thereto, such as position R743 of SEQ ID NO:2 (Zea mays sequence), position R745 of SEQ ID NO:274 (Oryza sativa sequence), position R758 of SEQ ID NO:10 (Helianthus annuus sequence), position R749 of SEQ ID NO:7 (Setaria italica sequence), position R765 of SEQ ID NO:3 (Nannochloropsis gaditana sequence), position R777 of SEQ ID NO:9 (Ostreococcus tauri sequence) or position S708 of SEQ ID NO:13 (Schizosaccharomyces japonicus sequence). The mutation may be a substitution of R. The substitution may be a non-conservative mutation. R may be substituted with an S or K residue. In such an embodiment, the BIO3-BIO1 enzyme may comprise or consist of a sequence according to SEQ ID NO: 53, 117, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position L786 of SEQ ID NO:1 (WT Arabidopsis thaliana sequence) or at a corresponding position thereto, such as position V773 of SEQ ID NO:2 (Zea mays sequence), position 1775 of SEQ ID NO:274 (Oryza sativa sequence), position L788 of SEQ ID NO:10 (Helianthus annuus sequence), position V779 of SEQ ID NO:7 (Setaria italica sequence), position V797 of SEQ ID NO:3 (Nannochloropsis gaditana sequence), position V806 of SEQ ID NO:9 (Ostreococcus tauri sequence) or position L736 of SEQ ID NO:13 (Schizosaccharomyces japonicus sequence). The mutation may be a substitution of L. The substitution may be a non-conservative mutation. L may be substituted with an S residue. In such an embodiment, the BIO3-BIO1 enzyme may comprise or consist of a sequence according to SEQ ID NO: 40, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position R790 of SEQ ID NO:1 (WT Arabidopsis thaliana sequence) or at a corresponding position thereto, such as position R777 of SEQ ID NO:2 (Zea mays sequence), position R779 of SEQ ID NO:274 (Oryza sativa sequence), position R792 of SEQ ID NO:10 (Helianthus annuus sequence), position R783 of SEQ ID NO:7 (Setaria italica sequence), position R801 of SEQ ID NO:3 (Nannochloropsis gaditana sequence), position R810 of SEQ ID NO:9 (Ostreococcus tauri sequence) or position R740 of SEQ ID NO:13 (Schizosaccharomyces japonicus sequence). The mutation may be a substitution of R. The substitution may be a non-conservative mutation. R may be substituted with a C residue. In such an embodiment, the BIO3-BIO1 enzyme may comprise or consist of a sequence according to SEQ ID NO: 118, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise an amino acid sequence that has a mutation at position R797 of SEQ ID NO:1 (WT Arabidopsis thaliana sequence) or at a corresponding position thereto, such as position R785 of SEQ ID NO:2 (Zea mays sequence), position and R787 of SEQ ID NO:274 (Oryza sativa sequence), position and R799 of SEQ ID NO:10 (Helianthus annuus sequence), position and R791 SEQ ID NO:7 (Setaria italica sequence), position R808 of SEQ ID NO:3 (Nannochloropsis gaditana sequence), position R817 of SEQ ID NO:9 (Ostreococcus tauri sequence) or position R754 of SEQ ID NO:13 (Schizosaccharomyces japonicus sequence). The mutation may be a substitution of R. The substitution may be a non-conservative mutation. R may be substituted with a Q residue. In such an embodiment, the BIO3-BIO1 enzyme may comprise or consist of a sequence according to SEQ ID NO: 119, or a sequence having at least at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity thereto, or a fragment thereof.

The modified BIO3-BIO1 enzyme may comprise any combination of more than one mutation described hereinabove, suitably two or more, three or more, four or more, five or more, six or more, seven or more, eight or more, nine or more, ten or more mutations, up to 21 mutations as listed above, suitably any combination of mutations as described hereinabove is envisaged.

In one embodiment, modified BIO3-BIO1 enzyme may comprise one or more of the following mutations selected from: C388D, C388T, A507S, F348C, F348N, F348S, F348T, F348V, G700A, G700S, P421L, P508T, R756S, S509V, S509W, W391G, and/or W392S of SEQ ID NO:1 (WT Arabidopsis thaliana sequence), or corresponding mutations thereto. Suitably, the corresponding mutations thereto may be those in any homologous sequence to that of SEQ ID NO:1, such as those in SEQ ID NOs 2-14, 271-276 and 319, for example as shown in Table 1.

In one embodiment, modified BIO3-BIO1 enzyme may comprise one or more of the following mutations selected from: C388T or C388D of SEQ ID NO:1 (WT Arabidopsis thaliana sequence), or corresponding mutations thereto. Suitably, the corresponding mutations thereto may be those in any homologous sequence to that of SEQ ID NO:1, such as those in SEQ ID NOs 2-14, 271-276 and 319, for example as shown in Table 1.

Suitably the BIO3-BIO1 enzyme may further comprise a transit peptide, suitably a mitochondrial or chloroplast transit peptide, suitably at the C terminus thereof. In one embodiment the transit peptide is a mitochondrial transit peptide.

Suitably such mitochondrial transit peptides are present in wild type BIO3-BIO1 enzymes. Suitably therefore the BIO3-BIO1 enzyme may comprise a native, endogenous mitochondrial transit peptide. Alternatively, the endogenous mitochondrial transit peptide may be replaced with a heterologous mitochondrial transit peptide, derived from a different BIO3-BIO1 enzyme, or a heterologous chloroplast transit peptide.

Suitably therefore in some embodiments, the BIO3-BIO1 enzyme may further be modified by the addition of a sequence. Suitably by the addition of a sequence to its N or C terminus. Suitably the sequence is a heterologous transit peptide. Suitably the heterologous transit peptide is added to the C terminus of the BIO3-BIO1 enzyme.

In one embodiment, therefore, the modified BIO3-BIO1 enzyme comprises a heterologous transit peptide, suitably a heterologous mitochondrial or chloroplast transit peptide, suitably at the C terminus thereof. In one embodiment the transit peptide is a heterologous mitochondrial transit peptide. Suitably the heterologous transit peptide is added to the BIO3-BIO1 enzyme, suitably such that the BIO3-BIO1 enzyme is produced as a fusion protein with the heterologous transit peptide.

Suitably the heterologous mitochondrial transit peptide is a plant mitochondrial transit peptide. Suitably the heterologous mitochondrial transit peptide may be derived from a heterologous BIO3-BIO1 enzyme, suitably from a wild type heterologous BIO3-BIO1 enzyme. Suitably the heterologous mitochondrial transit peptide may be derived from a heterologous plant BIO3-BIO1 enzyme, suitably from any of the BIO3-BIO1 enzymes defined herein.

Suitable mitochondrial transit peptides may be selected from: SEQ ID NO: 200 (MTP) for example, or from any of the underlined sequences present in the BIO3-BIO1 enzyme sequences of SEQ ID NO: 1, 2, 4, 5, 7, 8, 9, 10, 11, 12, 14, 271, 272, 273, 274, 275, 276, or 319.

Suitably therefore the MTP may also be selected from any of the following sequences (underlined parts of SEQ ID NO: 1, 2, 4, 5, 7, 8, 9, 10, 11, 12, 14, 271, 272, 273, 274, 275, and 276):

(SEQ ID NO: 277)
MIPVTATLIRHRLRHLRHRIRF
(SEQ ID NO: 278)
MVRRLFLHHARRRLHSGTASSIPLSTPIFAIFGANTGVGKTLVSAGLATA
LLSSPSPAVSSVAYLKPLQTGYPVDSDASFVFSRTPALLRAFPPCRATRL
IASCRTFF
(SEQ ID NO: 279)
MASLALTRRRFNPVSRLSLHKCRSTF
(SEQ ID NO: 280)
MHLLLLLPLRRRCTNPIAPRIAHQSRFLVSTAGACSPLPRHLLSGIWGR
CL
(SEQ ID NO: 281)
MLPRLLLRSRHRRRY
(SEQ ID NO: 282)
MPSCPLLPLLPRRRPPPSPLLLLLLSRSLHSS
(SEQ ID NO: 283)
MSAPIARRASSVARGRTRWLTSTSIERSREWFVRS
(SEQ ID NO: 284)
MFHPPLLFTLSRHFLRRHHHRH
(SEQ ID NO: 285)
MYFLISLHRRGHRHLRHVKDL
(SEQ ID NO: 286)
MLTLSMRMRPRTLMKMSFSTRAGLGQLGCLT
(SEQ ID NO: 287)
MAPVPALLWRSLRTHQVYGANTDVGKTIFSTVL
(SEQ ID NO: 288)
MLRRLLLRHARRLHY
(SEQ ID NO: 289)
MSPVTATLLRHRLRHLRHHHIRLN
(SEQ ID NO: 290)
MLLRRHHHHRLFRLRLLLLHHHHHQFQSF
(SEQ ID NO: 291)
MLRLLRHARRH
(SEQ ID NO: 292)
MFRFPSVLLSRRLHRREL
(SEQ ID NO: 293)
MLRRLLLRQARRRHCSSSFSSALP

Suitable MTPs in any BIO3-BIO1 enzyme sequence may be determined by using TargetP-2.0 https://services.healthtech.dtu.dk/service.php?TargetP, as described in Jos6 Juan Almagro Armenteros et al. Life Science Alliance 2 (5), e201900429. doi:10.26508/lsa.201900429

A mitochondrial transit peptide used in a modified BIO3-BIO1 enzyme may have at least 60% sequence identity to SEQ ID NO: 200, or to any of the sequences of SEQ ID NO: 277 to 293. For example, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO:200, or any of the sequences of SEQ ID NO: 277 to 293.

Suitably, the modified BIO3-BIO1 enzyme may comprise an amino acid sequence that has at least 30% sequence identity to an amino acid sequence according to any of SEQ ID NOs 1 to 14, 271-276 and 319, (or SEQ ID NO: 294 to 310 and 320), fused to a heterologous mitochondrial transit peptide, such as for example SEQ ID NO:200, or any of the sequences of SEQ ID NO: 277 to 394. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to any of SEQ ID NO: 1-14, 271-276 and 319, (or SEQ ID NO: 294 to 310 and 320), fused to a heterologous mitochondrial transit peptide such as for example SEQ ID NO:200, or any of the sequences of SEQ ID NO: 277 to 293.

Suitably, the modified BIO3-BIO1 enzyme may comprise an amino acid sequence that has at least 30% sequence identity to an amino acid sequence according to any of SEQ ID NOs. 347 to 487. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to any of SEQ ID NO: 347 to 487.

### Modified BioA Enzyme

As described above, embodiments of the invention comprise a non-modified BioA enzyme. Suitably a wild type BioA enzyme may be used.

Suitably in such embodiments, the BioA enzyme may comprise an amino acid sequence that has at least 30% identity to an amino acid sequence according to any of SEQ ID NOs: 159 to 199. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 159 to 199.

In one embodiment, the BioA enzyme is not an E. coli BioA enzyme. In one embodiment, therefore, the BioA enzyme may comprise an amino acid sequence that has at least 30% identity to an amino acid sequence according to any of SEQ ID NOs: 160 to 199. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 160 to 199. In one embodiment, therefore, the BioA enzyme may comprise an amino acid sequence that has at least 70% identity to an amino acid sequence according to any of SEQ ID NOs: 160 to 199. In one embodiment, therefore, the BioA enzyme may comprise an amino acid sequence that has at least 80% identity to an amino acid sequence according to any of SEQ ID NOs: 160 to 199. In one embodiment, therefore, the BioA enzyme may comprise an amino acid sequence that has at least 90% identity to an amino acid sequence according to any of SEQ ID NOs: 160 to 199. In one embodiment, therefore, the BioA enzyme may comprise an amino acid sequence that has at least 95% identity to an amino acid sequence according to any of SEQ ID NOs: 160 to 199.

Suitably in such embodiments, the BioA enzyme may comprise an amino acid sequence that has at least 30% identity to an amino acid sequence according to any of SEQ ID NOs: 167. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 167.

Suitably in such embodiments, the BioA enzyme may comprise an amino acid sequence that has at least 30% identity to an amino acid sequence according to any of SEQ ID NOs: 169. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 169.

Suitably in such embodiments, the BioA enzyme may comprise an amino acid sequence that has at least 30% identity to an amino acid sequence according to any of SEQ ID NOs: 170. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 170.

Suitably in such embodiments, the BioA enzyme may comprise an amino acid sequence that has at least 30% identity to an amino acid sequence according to any of SEQ ID NOs: 171. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 171.

Suitably in such embodiments, the BioA enzyme may comprise an amino acid sequence that has at least 30% identity to an amino acid sequence according to any of SEQ ID NOs: 184. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 184.

Suitably in such embodiments, the BioA enzyme may comprise an amino acid sequence that has at least 30% identity to an amino acid sequence according to any of SEQ ID NOs: 166. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 166.

Suitably in such embodiments, the BioA enzyme may comprise an amino acid sequence that has at least 30% identity to an amino acid sequence according to any of SEQ ID NOs: 181. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 181.

Suitably in such embodiments, the BioA enzyme may comprise an amino acid sequence that has at least 30% identity to an amino acid sequence according to any of SEQ ID NOs: 180. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 180.

Suitably in such embodiments, the BioA enzyme may comprise an amino acid sequence that has at least 30% identity to an amino acid sequence according to any of SEQ ID NOs: 184. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% identity to an amino acid sequence according to any of SEQ ID NO: 184.

In one embodiment, therefore, the BioA enzyme may comprise an amino acid sequence that has at least 70% identity to an amino acid sequence according to SEQ ID NOs: 167 or 170. In one embodiment, therefore, the BioA enzyme may comprise an amino acid sequence that has at least 80% identity to an amino acid sequence according to SEQ ID NOs: 167 or 170. In one embodiment, therefore, the BioA enzyme may comprise an amino acid sequence that has at least 90% identity to an amino acid sequence according to SEQ ID NOs: 167 or 170. In one embodiment, therefore, the BioA enzyme may comprise an amino acid sequence that has at least 95% identity to an amino acid sequence according to SEQ ID NOs: 167 or 170.

In any aspect of the invention as described herein, a BioA enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NOs 159 to 199 herein, which may also be referred to as reference sequences.

In any aspect of the invention as described herein, a BioA enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NO 167 herein, which may also be referred to as reference sequences.

In any aspect of the invention as described herein, a BioA enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NO 169 herein, which may also be referred to as reference sequences.

In any aspect of the invention as described herein, a BioA enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NO 170 herein, which may also be referred to as reference sequences.

In any aspect of the invention as described herein, a BioA enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NO 171 herein, which may also be referred to as reference sequences.

In any aspect of the invention as described herein, a BioA enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NO 184 herein, which may also be referred to as reference sequences.

In any aspect of the invention as described herein, a BioA enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NO 166 herein, which may also be referred to as reference sequences.

In any aspect of the invention as described herein, a BioA enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NO 181 herein, which may also be referred to as reference sequences.

In any aspect of the invention as described herein, a BioA enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NO 180 herein, which may also be referred to as reference sequences.

In any aspect of the invention as described herein, a BioA enzyme which has resistance to a compound which inhibits the biotin synthesis pathway, whether partial or complete, may comprise an amino acid which has at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to any of SEQ ID NO 185 herein, which may also be referred to as reference sequences.

A BioA enzyme encoded by a nucleic acid or a BioA enzyme of the invention may be a functional fragment of a BioA enzyme as described herein. A “functional fragment” refers to a protein fragment that retains protein function. As such, a functional fragment of an BioA enzyme is a fragment, portion or part of a BioA protein that is capable of catalysing the conversion of KAPA to 7,8 Diaminopelargonic Acid (DAPA).

In any aspect of the invention, a BioA enzyme which has resistance to a compound which inhibits the biotin synthesis pathway may be a homologue of any of SEQ ID NOs 159 to 199. Suitably, a BioA enzyme which has resistance to a compound which inhibits the biotin synthesis pathway may be a homologue of any of SEQ ID NO 167. Suitably, a BioA enzyme which has resistance to a compound which inhibits the biotin synthesis pathway may be a homologue of any of SEQ ID NO 169. Suitably, a BioA enzyme which has resistance to a compound which inhibits the biotin synthesis pathway may be a homologue of any of SEQ ID NO 170. Suitably, a BioA enzyme which has resistance to a compound which inhibits the biotin synthesis pathway may be a homologue of any of SEQ ID NO 171. Suitably, a BioA enzyme which has resistance to a compound which inhibits the biotin synthesis pathway may be a homologue of any of SEQ ID NO 184. Suitably, a BioA enzyme which has resistance to a compound which inhibits the biotin synthesis pathway may be a homologue of any of SEQ ID NO 166. Suitably, a BioA enzyme which has resistance to a compound which inhibits the biotin synthesis pathway may be a homologue of any of SEQ ID NO 181. Suitably, a BioA enzyme which has resistance to a compound which inhibits the biotin synthesis pathway may be a homologue of any of SEQ ID NO 180. Suitably, a BioA enzyme which has resistance to a compound which inhibits the biotin synthesis pathway may be a homologue of any of SEQ ID NO 185. As used herein, “homologue” refers to a protein that is functionally equivalent i.e. has the same enzymatic activity as an enzyme having an amino acid sequence according to SEQ ID NO 159 to 199 (i.e. acts as a BioA enzyme as defined herein), but may have a limited number of amino acid substitutions, deletions, insertions or additions in the amino acid sequence. Homologues may have lower sequences identities, for example at least 20%, at least 25%, at least 30%, at least 35% or at least 40% or more sequence identity to a BioA enzyme identified herein, but are capable of carrying out the same enzymatic reaction. The invention therefore includes any isoforms of BioA enzymes and their mutations as defined herein.

In one embodiment, the BioA enzyme is derived from E. coli and may comprise an amino acid sequence that has at least 70%, at least 75%, at least 80%, at least 85%, at least 90%, at least 91%, at least 92%, at least 93%, at least 94%, at least 95%, at least 98% or at least 99% identity to an amino acid sequence according to SEQ ID NO: 159. In one embodiment, the BioA enzyme is derived from E. coli and consists of an amino acid sequence according to SEQ ID NO:159.

In one embodiment, the BioA enzyme is derived from Pantoea ananatis and may comprise an amino acid sequence that has at least 70%, at least 75%, at least 80%, at least 85%, at least 90%, at least 91%, at least 92%, at least 93%, at least 94%, at least 95%, at least 98% or at least 99% identity to an amino acid sequence according to SEQ ID NO: 167. In one embodiment, the BioA enzyme is derived from Pantoea ananatis and consists of an amino acid sequence according to SEQ ID NO:167.

In one embodiment, the BioA enzyme is derived from Streptomyces hygroscopicus and may comprise an amino acid sequence that has at least 70%, at least 75%, at least 80%, at least 85%, at least 90%, at least 91%, at least 92%, at least 93%, at least 94%, at least 95%, at least 98% or at least 99% identity to an amino acid sequence according to SEQ ID NO: 169. In one embodiment, the BioA enzyme is derived from Streptomyces hygroscopicus and consists of an amino acid sequence according to SEQ ID NO:169.

In one embodiment, the BioA enzyme is derived from Streptomyces viridochromogenes and may comprise an amino acid sequence that has at least 70%, at least 75%, at least 80%, at least 85%, at least 90%, at least 91%, at least 92%, at least 93%, at least 94%, at least 95%, at least 98% or at least 99% identity to an amino acid sequence according to SEQ ID NO: 170. In one embodiment, the BioA enzyme is derived from Streptomyces viridochromogenes and consists of an amino acid sequence according to SEQ ID NO:170.

In one embodiment, the BioA enzyme is derived from Stenotrophomonas maltophilia and may comprise an amino acid sequence that has at least 70%, at least 75%, at least 80%, at least 85%, at least 90%, at least 91%, at least 92%, at least 93%, at least 94%, at least 95%, at least 98% or at least 99% identity to an amino acid sequence according to SEQ ID NO: 171. In one embodiment, the BioA enzyme is derived from Stenotrophomonas maltophilia and consists of an amino acid sequence according to SEQ ID NO:171.

In one embodiment, the BioA enzyme is derived from Chroococcidiopsis sp. CCMEE 29 and may comprise an amino acid sequence that has at least 70%, at least 75%, at least 80%, at least 85%, at least 90%, at least 91%, at least 92%, at least 93%, at least 94%, at least 95%, at least 98% or at least 99% identity to an amino acid sequence according to SEQ ID NO: 184. In one embodiment, the BioA enzyme is derived from Chroococcidiopsis sp. CCMEE 29 and consists of an amino acid sequence according to SEQ ID NO:184.

In one embodiment, the BioA enzyme is derived from Bacillus subtilis and may comprise an amino acid sequence that has at least 70%, at least 75%, at least 80%, at least 85%, at least 90%, at least 91%, at least 92%, at least 93%, at least 94%, at least 95%, at least 98% or at least 99% identity to an amino acid sequence according to SEQ ID NO: 166. In one embodiment, the BioA enzyme is derived from Bacillus subtilis and consists of an amino acid sequence according to SEQ ID NO:166.

In one embodiment, the BioA enzyme is derived from Chitinophaga filiformis and may comprise an amino acid sequence that has at least 70%, at least 75%, at least 80%, at least 85%, at least 90%, at least 91%, at least 92%, at least 93%, at least 94%, at least 95%, at least 98% or at least 99% identity to an amino acid sequence according to SEQ ID NO: 180. In one embodiment, the BioA enzyme is derived from Chitinophaga filiformis and consists of an amino acid sequence according to SEQ ID NO:180.

In one embodiment, the BioA enzyme is derived from Tenacibaculum adriaticum and may comprise an amino acid sequence that has at least 70%, at least 75%, at least 80%, at least 85%, at least 90%, at least 91%, at least 92%, at least 93%, at least 94%, at least 95%, at least 98% or at least 99% identity to an amino acid sequence according to SEQ ID NO: 185. In one embodiment, the BioA enzyme is derived from Tenacibaculum adriaticum and consists of an amino acid sequence according to SEQ ID NO: 185.

However, in some embodiments the BioA enzyme may be modified. Suitably the BioA enzyme may comprise one or more modifications, which may be insertions, deletions, additions or substitutions as described hereinabove. In another aspect of the invention there is also provided a modified BioA enzyme.

Suitably the BioA enzyme is modified by the addition of a sequence. Suitably by the addition of a sequence to its N or C terminus. Suitably the sequence is a transit peptide. Suitably the transit peptide is added to the C terminus of the BioA enzyme.

In one embodiment, therefore, the modified BioA enzyme comprises a transit peptide, suitably a mitochondrial or chloroplast transit peptide, suitably at the C terminus thereof. In one embodiment the transit peptide is a mitochondrial transit peptide. Suitably the transit peptide is added to the BioA enzyme, suitably such that the BioA enzyme is produced as a fusion protein with the transit peptide.

Suitably the mitochondrial transit peptide is a plant mitochondrial transit peptide. Suitably therefore the mitochondrial transit peptide is heterologous to the BioA enzyme. Suitably the mitochondrial transit peptide may be derived from a BIO3-BIO1 enzyme, suitably from a wild type BIO3-BIO1 enzyme. Suitably the mitochondrial transit peptide may be derived from a plant BIO3-BIO1 enzyme, suitably from any of the BIO3-BIO1 enzymes defined herein.

Suitable mitochondrial transit peptides may be selected from: SEQ ID NO: 200 (MTP) for example, or from any of the underlined sequences present in the BIO3-BIO1 enzyme sequences of SEQ ID NO: 1, 2, 4, 5, 7, 8, 9, 10, 11, 12, 14, 271, 272, 273, 274, 275, or 276.

Suitably therefore the MTP may also be selected from any of the following sequences (underlined parts of SEQ ID NO: 1, 2, 4, 5, 7, 8, 9, 10, 11, 12, 14, 271, 272, 273, 274, 275, and 276):

(SEQ ID NO: 277)
MIPVTATLIRHRLRHLRHRIRF
(SEQ ID NO: 278)
MVRRLFLHHARRRLHSGTASSIPLSTPIFAIFGANTGVGKTLVSAGLATA
LLSSPSPAVSSVAYLKPLQTGYPVDSDASFVFSRTPALLRAFPPCRATRL
IASCRTFF
(SEQ ID NO: 279)
MASLALTRRRFNPVSRLSLHKCRSTF
(SEQ ID NO: 280)
MHLLLLLPLRRRCTNPIAPRIAHQSRFLVSTAGACSPLPRHLLSGIWGR
CL
(SEQ ID NO: 281)
MLPRLLLRSRHRRRY
(SEQ ID NO: 282)
MPSCPLLPLLPRRRPPPSPLLLLLLSRSLHSS
(SEQ ID NO: 283)
MSAPIARRASSVARGRTRWLTSTSIERSREWFVRS
(SEQ ID NO: 284)
MFHPPLLFTLSRHFLRRHHHRH
(SEQ ID NO: 285)
MYFLISLHRRGHRHLRHVKDL
(SEQ ID NO: 286)
MLTLSMRMRPRTLMKMSFSTRAGLGQLGCLT
(SEQ ID NO: 287)
MAPVPALLWRSLRTHQVYGANTDVGKTIFSTVL
(SEQ ID NO: 288)
MLRRLLLRHARRLHY
(SEQ ID NO: 289)
MSPVTATLLRHRLRHLRHHHIRLN
(SEQ ID NO: 290)
MLLRRHHHHRLFRLRLLLLHHHHHQFQSF
(SEQ ID NO: 291)
MLRLLRHARRH
(SEQ ID NO: 292)
MFRFPSVLLSRRLHRREL
(SEQ ID NO: 293)
MLRRLLLRQARRRHCSSSFSSALP.

A mitochondrial transit peptide used in a modified BioA enzyme may have at least 60% sequence identity to SEQ ID NO: 200, or to any of the sequences of SEQ ID NO: 277 to 293. For example, at least 60%, at least 61%, at least 62%, at least 63%, at least 64%, at least 65%, at least 66%, at least 67%, at least 68%, at least 69%, least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identity to SEQ ID NO:200, or to any of the sequences of SEQ ID NO: 277 to 293.

Suitably, the modified BioA enzyme may comprise an amino acid sequence that has at least 30% sequence identity to an amino acid sequence according to any of SEQ ID NOs 159-199 fused to a mitochondrial transit peptide according to SEQ ID NO:200, or to any of the sequences of SEQ ID NO: 277 to 293. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to any of SEQ ID NO: 159-199 fused to SEQ ID NO:200, or to any of the sequences of SEQ ID NO: 277 to 293.

Suitably, the modified BioA enzyme may comprise an amino acid sequence that has at least 30% sequence identity to an amino acid sequence according to any of SEQ ID NOs 160-199 fused to a mitochondrial transit peptide according to SEQ ID NO:200, or to any of the sequences of SEQ ID NO: 277 to 293. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to any of SEQ ID NO: 160-199 fused to SEQ ID NO:200, or to any of the sequences of SEQ ID NO: 277 to 293.

In one embodiment, the modified BioA enzyme may comprise an amino acid sequence that has at least 30% sequence identity to SEQ ID NO: 201 (E. coli BioA with MTP). Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 201.

Suitably, the modified BioA enzyme may comprise an amino acid sequence that has at least 30% sequence identity to an amino acid sequence according to SEQ ID NO: 167 fused to a mitochondrial transit peptide according to SEQ ID NO:200, or to any of the sequences of SEQ ID NO: 277 to 293. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 167 fused to SEQ ID NO:200, or to any of the sequences of SEQ ID NO: 277 to 293.

Suitably, the modified BioA enzyme may comprise an amino acid sequence that has at least 30% sequence identity to an amino acid sequence according to SEQ ID NO: 169 fused to a mitochondrial transit peptide according to SEQ ID NO:200, or to any of the sequences of SEQ ID NO: 277 to 293. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 169 fused to SEQ ID NO:200, or to any of the sequences of SEQ ID NO: 277 to 293.

Suitably, the modified BioA enzyme may comprise an amino acid sequence that has at least 30% sequence identity to an amino acid sequence according to SEQ ID NO: 170 fused to a mitochondrial transit peptide according to SEQ ID NO:200, or to any of the sequences of SEQ ID NO: 277 to 293. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 170 fused to SEQ ID NO:200, or to any of the sequences of SEQ ID NO: 277 to 293.

Suitably, the modified BioA enzyme may comprise an amino acid sequence that has at least 30% sequence identity to an amino acid sequence according to SEQ ID NO: 171 fused to a mitochondrial transit peptide according to SEQ ID NO:200, or to any of the sequences of SEQ ID NO: 277 to 293. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 171 fused to SEQ ID NO:200, or to any of the sequences of SEQ ID NO: 277 to 293.

Suitably, the modified BioA enzyme may comprise an amino acid sequence that has at least 30% sequence identity to an amino acid sequence according to SEQ ID NO: 184 fused to a mitochondrial transit peptide according to SEQ ID NO:200, or to any of the sequences of SEQ ID NO: 277 to 293. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 184 fused to SEQ ID NO:200, or to any of the sequences of SEQ ID NO: 277 to 293.

Suitably, the modified BioA enzyme may comprise an amino acid sequence that has at least 30% sequence identity to an amino acid sequence according to SEQ ID NO: 166 fused to a mitochondrial transit peptide according to SEQ ID NO:200, or to any of the sequences of SEQ ID NO: 277 to 293. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 166 fused to SEQ ID NO:200, or to any of the sequences of SEQ ID NO: 277 to 293.

Suitably, the modified BioA enzyme may comprise an amino acid sequence that has at least 30% sequence identity to an amino acid sequence according to SEQ ID NO: 180 fused to a mitochondrial transit peptide according to SEQ ID NO:200, or to any of the sequences of SEQ ID NO: 277 to 293. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 180 fused to SEQ ID NO:200, or to any of the sequences of SEQ ID NO: 277 to 293.

Suitably, the modified BioA enzyme may comprise an amino acid sequence that has at least 30% sequence identity to an amino acid sequence according to SEQ ID NO: 185 fused to a mitochondrial transit peptide according to SEQ ID NO:200, or to any of the sequences of SEQ ID NO: 277 to 293. Other variants of such an enzyme may comprise an amino acid sequence of at least 35%, at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 98% or at least 99% sequence identity to an amino acid sequence according to SEQ ID NO: 185 fused to SEQ ID NO:200, or to any of the sequences of SEQ ID NO: 277 to 293.

The modified BioA enzyme may optionally comprise other modifications, suitably the BioA enzyme may comprise one or more mutations. Suitably the BioA enzyme may comprise one or more mutations in the BioA motifs identified above. By mutation is meant any substitution, deletion or insertion of 1, 2, 3, 4, 5, 6, 7, 8, 9, 10 or more amino acids. Modified BioA enzymes of the invention may comprise such a mutation at any one or more positions of any of the motifs identified above. In one embodiment, BioA enzymes of the invention may comprise a substitution mutation at any one or more of the positions of:

Motif 15
(SEQ ID NO: 268)
(A/G/S); (F/Y); H; G; (D/E); T; (F/I/L/M/V/W);
(A/D/E/G/K/M/Q); (A/G/P/T); (I/L/M/V); (A/E/S);
(A/I/L/T/V),
and/or
Motif 16
(SEQ ID NO: 269)
D; E; (I/V); (A/F/L/M); (T/V); G; (F/L/W); G;
(K/R); (C/S/T); G; (A/E/K/L/Q/P/R/S/T);
(F/L/M/N/R/W); F; (A/G/S).

Suitably such mutations may be in addition to or alternative to the targeting peptide modification.

Suitable amino acid substitutions may be those that confer an increased resistance to biotin-pathway inhibitors. Suitable mutations may be conserved or non-conserved as defined elsewhere herein. Suitable means to screen for such mutations are described elsewhere herein.

Combined Resistance and/or Additional Traits of Interest

The plants or parts thereof of the invention may further be modified to comprise an additional trait of interest. In one embodiment, the additional trait of interest increases resistance to a different compound which inhibits a different plant metabolic process. Suitably, the plants or parts thereof of the invention may further be modified to comprise increased resistance to a compound which inhibits a plant metabolic process other than the biotin synthesis pathway. Suitably this may be regarded as ‘stacking’ of resistance. Suitably resistance to a compound which inhibits the biotin synthesis pathway may be stacked with resistance to another compound which inhibits a different metabolic pathway, in the plants of the invention. Suitably therefore the plant or part thereof exhibits a second compound-resistant trait.

Suitably the plants or parts thereof of the invention may further be modified to comprise increased resistance to a compound which inhibits a different plant metabolic process to that of the biotin synthesis pathway. Suitably the plants or parts thereof of the invention may further be modified to comprise increased resistance to a compound which inhibits an essential plant metabolic process. By inhibiting a plant metabolic process, this may mean inhibiting one or more enzymes of a plant metabolic process. Suitably the plants or parts thereof of the invention may further be modified to comprise increased resistance to a compound which is not a compound that targets the biotin synthesis pathway, but which targets a different essential plant metabolic process.

Suitably the plant or part thereof may comprise an additional modified enzyme, suitably which has been modified to increase its resistance to the compound which inhibits a different plant metabolic process to that of the biotin synthesis pathway. Suitably such compounds are herbicides. Suitably the plant or part thereof may comprise increased resistance to a herbicide which inhibits the biotin synthesis pathway and increased resistance to another herbicide, suitably which inhibits a different plant metabolic process. Suitably therefore the plant or part thereof exhibits a second herbicide-resistant trait.

The BioA and BIO3-BIO1 enzymes and variants thereof provided herein can therefore be stacked with one or more additional modified enzymes which confers a desirable trait such as, for example, insect, disease or herbicide resistance or other desirable agronomic traits of interest including, but not limited to, traits associated with high oil content; traits associated with short stature, increase protein content, increased digestibility; balanced amino acid content; improved drought resistance, modified maturity and/or flowering time, and high energy content. Such traits may refer to properties of both seed and non-seed plant tissues, or to food or feed prepared from plants or seeds having such traits.

As used herein, gene or trait “stacking” comprises combining desired genes or traits into one transgenic plant line. Stacking can include the introduction of transgenic traits of interest, genome edited traits of interest or native traits of interest. The additional polynucleotide can be introduced by a variety of approaches, as described herein in relation to polynucleotides encoding the BIO3-BIO1 and BioA enzymes, including by transgenic means, by breeding, by genome editing or by cisgenesis. As one approach, plant breeders stack transgenic traits by making crosses between parents that each have a desired trait and then identifying offspring that have both of these desired traits (so-called “breeding stacks”). Another way to stack genes is by transferring two or more genes into the cell nucleus of a plant at the same time during transformation. In embodiments, the two or more genes may be transferred via distinct expression cassettes or via a common expression cassette. Another way to stack genes is by re-transforming a transgenic plant comprising a desired trait with another gene of interest conferring another desired trait to thereby provide a progeny transgenic plant comprising the combination of traits. Such methods can include, for example, random integration techniques or targeted integration via a gene editing system such as Crispr or meganucleases. For example, gene stacking can be used to combine an herbicide resistant trait disclosed herein, with one or more of an insect resistance trait, an additional herbicide resistant trait, an, agronomic performance trait (such as short stature corn), or a disease resistance trait. The use of a selectable marker in addition to a gene of interest would also be considered gene stacking. In embodiments, the offspring or progeny plant having the desired combination of traits is identified through the use of genetic markers or molecular markers including but not limited to SNPs, QTLs, primers or probes directed to desired trait-associated genes or transgenes, promoters, microRNAs, siRNAs, mRNAs, ds RNAs, transcriptional profiles, and methylation patterns.

As used herein, “cisgenic” or “cisgenesis” involves the insertion, optionally into a genome (e.g., a plant genome), of one or more genes of the same or a related species, or from a crossable donor. As used herein a “cisgenic construct” is a recombinant nucleic acid sequence present in a cell, and optionally integrated into the cell's genome, wherein the recombinant nucleic acid sequence comprises a native gene comprising a regulatory element operably linked to a nucleic acid sequence for a gene of interest, wherein the regulatory element and gene of interest are both native to the plant, or from a related species, or from a crossable donor, and are operably linked in the native cell at a genomic location different from the genomic location where they are integrated as the cisgenic construct. In other embodiments, the cisgenic construct comprises the native genomic sequence of the gene of interest (native regulatory element and native coding region), wherein the native genomic sequence has been modified by one or more gene edit.

As such, in some embodiments, the BIO3-BIO1 polypeptide can be deployed as a “cisgenic construct”. Such cisgenic constructs are integrated into the genome in a heterologous location (that is, a location different from the native location in the genome). In such instances, the cisgenic constructs encoding the BIO3-BIO1polypeptide comprises one or more gene edits that increase resistance to a herbicide upon expression in a plant. The cisgenic construct can be stably integrated into the genome via any method, including for example, targeted integration or random integration. In one non-limiting embodiment, the cisgenic construct comprise the native genomic sequence of the BIO3-BIO1 polypeptide from corn, soybean, sunflower, rice, or wheat having at least one gene edit that increase resistance to a herbicide as disclosed herein.

The cisgenic construct when integrated via targeted insertion can be deployed to neighbour either native traits of interest and/or other heterologous traits of interest, such as herbicide tolerance traits or insect control traits or other traits of interest as disclosed herein.

In some embodiments, a polynucleotide or vector described herein, can include an additional coding sequence for one or more polypeptides or double stranded RNA molecules (dsRNA) of interest for agronomic traits that primarily are of benefit to a seed company, grower or grain processor. A polypeptide of interest can be any polypeptide encoded by a nucleotide sequence of interest, such as an enzyme. Non-limiting examples of polypeptides of interest that are suitable for production in plants include those resulting in agronomically important traits such as herbicide resistance (also sometimes referred to as “herbicide tolerance”), disease resistance, virus resistance, bacterial pathogen resistance, insect resistance, nematode resistance, or fungal resistance, such as modified enzymes conferring these traits. See, e.g., U.S. Pat. Nos. 5,569,823; 5,304,730; 5,495,071; 6,329,504; and 6,337,431. The polypeptide also can be one that increases plant vigor or yield (including traits that allow a plant to grow at different temperatures, soil conditions and levels of sunlight and precipitation), or one that allows identification of a plant exhibiting a trait of interest (e.g., a selectable marker, seed coat color, relative maturity group, etc.). Various polypeptides of interest, as well as methods for introducing these polypeptides into a plant, are described, for example, in U.S. Pat. Nos. 4,761,373; 4,769,061; 4,810,648; 4,940,835; 4,975,374; 5,013,659; 5,162,602; 5,276,268; 5,304,730; 5,495,071; 5,554,798; 5,561,236; 5,569,823; 5,767,366; 5,879,903, 5,928,937; 6,084,155; 6,329,504 and 6,337,431; as well as US Patent Publication No. 2001/0016956.

Suitably the additional polypeptide of interest or modified polypeptide such as an enzyme may comprise any known polypeptide of interest or modified polypeptide such as an enzyme in the art that increases resistance to a known compound which inhibits a plant metabolic process. Suitably such compounds are herbicides. Suitably therefore the additional polypeptide of interest or modified polypeptide such as an enzyme may comprise any known polypeptide of interest or modified polypeptide such as an enzyme in the art that provides an additional herbicide resistance trait, suitably by providing increased herbicide resistance. Suitably the plant or part thereof may comprise a modified or unmodified BIO3-BIO1 and/or BioA enzyme as disclosed herein, which provides increased resistance to a herbicide which inhibits the biotin synthesis pathway and an additional polypeptide of interest or modified polypeptide such as an enzyme which provides increased resistance to another herbicide, suitably which inhibits a different plant metabolic process.

Polynucleotides, suitably which encode a polypeptide of interest or modified polypeptide of interest such as an enzyme, conferring resistance/tolerance to a herbicide that inhibits the growing point or meristem, such as an imidazalinone or a sulfonylurea may be suitable in some embodiments. Exemplary polynucleotides in this category code for mutant ALS and AHAS enzymes as described, e.g., in U.S. Pat. Nos. 5,767,366 and 5,928,937. U.S. Pat. Nos. 4,761,373 and 5,013,659 are directed to plants resistant to various imidazalinone or sulfonamide herbicides. U.S. Pat. No. 4,975,374 relates to plant cells and plants containing a nucleic acid encoding a modified glutamine synthetase (GS) enzyme resistant to inhibition by herbicides that are known to inhibit GS, e.g., phosphinothricin and methionine sulfoximine. U.S. Pat. No. 5,162,602 discloses plants resistant to inhibition by cyclohexanedione and aryloxyphenoxypropanoic acid herbicides. The resistance is conferred by a modified acetyl coenzyme A carboxylase (ACCase) enzyme. Additional herbicide tolerant traits that confer tolerance to ACCase inhibitors can be found in WO2014144951, WO2017138986, WO2018205995, WO2021088601, WO2011028836, and WO2011028833.

Polypeptides, encoded by polynucleotide sequences conferring resistance to glyphosate are also suitable for the disclosure. See, e.g., U.S. Pat. Nos. 4,940,835 and 4,769,061. U.S. Pat. No. 5,554,798 discloses transgenic glyphosate resistant maize plants, which resistance is conferred by a modified 5-enolpyruvyl-3-phosphoshikimate (EPSP) synthase gene. Polynucleotides coding for resistance to phosphono compounds such as glufosinate ammonium or phosphinothricin, and pyridinoxy or phenoxy propionic acids and cyclohexones are also suitable. See, European Patent Application No. 0 242 246. See also, U.S. Pat. Nos. 5,879,903, 5,276,268 and 5,561,236.

Other suitable polynucleotides which confer resistance to herbicides that inhibit photosynthesis, such as a triazine and a benzonitrile (nitrilase) See, U.S. Pat. No. 4,810,648. Additional suitable polynucleotides coding for herbicide resistance include those coding for resistance to 2,2-dichloropropionic acid, sethoxydim, haloxyfop, imidazolinone herbicides, sulfonylurea herbicides, triazolopyrimidine herbicides, s-triazine herbicides and bromoxynil. Also suitable are polynucleotides encoding modified enzymes that confer resistance to a protox enzyme, or that provide enhanced resistance to plant diseases; enhanced tolerance of adverse environmental conditions (abiotic stresses) including but not limited to drought, excessive cold, excessive heat, or excessive soil salinity or extreme acidity or alkalinity; and alterations in plant architecture or development, including changes in developmental timing. See, e.g., U.S. Patent Publication No. 2001/0016956 and U.S. Pat. No. 6,084,155.

Additional herbicide tolerant traits include, PPO tolerant traits including, for example, one or more PPO enzyme trait set forth in US20190062777, U.S. Ser. No. 10/370,677, U.S. Ser. No. 11/124,803, WO2017217793, WO2020251313, U.S. Ser. No. 10/392,630, U.S. Ser. No. 10/378,023, WO2016099153, WO2019117579, WO2019117578, and U.S. Ser. No. 10/100,329, each of which is herein incorporated by reference in their entirety. HPPD tolerant traits include, for example, WO2009144079, U.S. Pat. No. 8,642,748, EP2453012, WO2013026740, U.S. Pat. No. 9,078,446, U.S. Ser. No. 10/793,872, U.S. Ser. No. 10/508,089, U.S. Ser. No. 10/400,249, U.S. Ser. No. 10/597,674, WO2018119364, WO2018119361, U.S. Ser. No. 11/180,770, US20200157086, US20210147866, U.S. Ser. No. 11/279,944, US202000331866, WO2019227036, WO2019227028, WO2022115296, and WO2011068567, each of which is herein incorporated by reference in their entirety. ACCase tolerant traits include, for example, US20120284812, US20120284853, US20160108423, US20160244780, US20160264990, US20170275645, US20210153448, U.S. Ser. No. 10/696,975B2, U.S. Ser. No. 10/370,678, CN109082416, U.S. Ser. No. 10/694,694, US20170265469, US20170231225, each of which is herein incorporated by reference. Additional herbicides tolerant traits of interest for stacking include glucosyl transferase enzymes as set forth in 2018213022 or solanesyl diphosphate synthase enzymes as set forth in WO2020236790, each of which is herein incorporated by reference in their entirety.

Disease resistance proteins such as enzymes that increase resistance to various plant disease including rust, include, but are not limited to, one or more of the various resistance genes set forth in: WO2019103918; WO202100878; WO2021022022; WO2021260673; WO2022173659; WO2022159341; WO2021154632A1, WO2021022026, WO2021022101, US20220135997; U.S. Ser. No. 10/842,097; or WO2022140257; each of which is incorporated by reference in their entirety. In other embodiments, the BioA or BIO3-BIO1 enzyme described herein or active variant or fragments thereof is stacked with a native trait that confers disease resistance. For example, the various intervals, locus or resistance genes as set forth in WO2009079729, U.S. Pat. No. 9,091,681, WO2010009404, WO2017222827, WO2021000878, WO2021022026, WO2021022101, WO2021154632, WO2022173659, (each of which is incorporated by reference in their entirety) can used to introduce a trait of interest. Disease resistance proteins and/or native traits that increase resistance to various plant diseases including NCLB include, for example, U.S. Pat. No. 8,921,646, US2021000059, U.S. Ser. No. 10/858,668, US20200199610, WO2022/013268, WO2022/013268, U.S. Pat. No. 9,040,772, U.S. Ser. No. 10/897,862, EP3839073, each of which is herein incorporated by reference.

Additional suitable polynucleotides include those coding for insecticidal polypeptides such as insecticidal enzymes. These polypeptides may be produced in amounts sufficient to control, for example, insect pests (i.e., insect controlling amounts). It is recognized that the amount of production of an insecticidal polypeptide in a plant necessary to control insects or other pests may vary depending upon the cultivar, type of pest, environmental factors and the like. Polynucleotides useful for additional insect or pest resistance include, for example, those that encode toxins identified in Bacillus organisms. Polynucleotides comprising nucleotide sequences encoding Bacillus thuringiensis (Bt) Cry proteins from several subspecies have been cloned and recombinant clones have been found to be toxic to lepidopteran, dipteran and/or coleopteran insect larvae. Examples of such Bt insecticidal proteins include the Cry proteins such as Cry1Aa, Cry1Ab, Cry1Ac, Cry1B, Cry1C, Cry1D, Cry1Ea, Cry1Fa, Cry3A, Cry9A, Cry9B, Cry⁹C, and the like, as well as vegetative insecticidal proteins such as Vip1, Vip2, Vip3, and the like. A full list of Bt-derived proteins can be found on the worldwide web at Bacillus thuringiensis Toxin Nomenclature Database maintained by the University of Sussex (see also, Crickmore et al. (1998) Microbiol. Mol. Biol. Rev. 62:807-813).

In embodiments, an additional polypeptide is an insecticidal polypeptide, such as an enzyme, derived from a non-Bt source, including without limitation, an alphα-amylase, a peroxidase, a cholesterol oxidase, a patatin, a protease, a protease inhibitor, a urease, an alphα-amylase inhibitor, a pore-forming protein, a chitinase, a lectin, an engineered antibody or antibody fragment, a Bacillus cereus insecticidal protein, a Xenorhabdus spp. (such as X. nematophila or X. bovienil) insecticidal protein, a Photorhabdus spp. (such as P. luminescens or P. asymobiotica) insecticidal protein, a Brevibacillus spp. (such as B. laterosporous) insecticidal protein, a Lysinibacillus spp. (such as L. sphearicus) insecticidal protein, a Chromobacterium spp. (such as C. subtsugae or C. piscinae) insecticidal protein, a Yersinia spp. (such as Y. entomophaga) insecticidal protein, a Paenibacillus spp. (such as P. propylaea) insecticidal protein, a Clostridium spp. (such as C. bifermentans) insecticidal protein, a Pseudomonas spp. (such as P. fluorescens) and a lignin.

In certain embodiments, the additional polypeptide or enzyme is a resistance protein such as an enzyme conferring enhanced pathogen resistance, such as enhanced resistance to any one of the following pathogens: soy cyst nematode, bacterial pustule, root knot nematode, frog eye leaf spot, phytopthora, brown stem rot, nematode, Asian Soybean Rust, smut, Golovinomyces cichoracearum, Erysiphe cichoracearum, Blumeria graminis, Podosphaera xanthii, Sphaerotheca fuliginea, Pythium ultimum, Uncinula necator, Mycosphaerella pinodes, Magnaporthe grisea, Bipolaris oryzae, Magnaporthe grisea, Rhizoctonia solani. Phytophthora sojae, Schizaphis graminum, Bemisia tabaci, Rhopalosiphum maidis, Deroceras reticulatum, Diatraea saccharalis, Schizaphis graminum, Myzus persicae, Sclerotinia sclerotiorum, Macrophomina phaseolina, or Fusarium virguliforme. Exemplary polynucleotides encoding proteins that confer increased pathogen resistance that may be stacked with the BIOA or BIO3-Biol enzymes of the invention include polynucleotides encoding proteins such as enzymes that confer increased ASR resistance as described in US Patent publication Nos. US 20200354739 and PCT Publications Nos. WO2019103918, WO2021154632A1, WO2021022022, WO2021022026, WO2021022101, WO2021260673, and WO2021263249, each of which is incorporated by reference in its entirety.

Polypeptides that are suitable for production in plants further include those that improve or otherwise facilitate the conversion of harvested plants or plant parts into a commercially useful product, including, for example, increased or altered carbohydrate content or distribution, improved fermentation properties, increased oil content, increased protein content, improved digestibility, and increased nutraceutical content, e.g., increased phytosterol content, increased tocopherol content, increased stanol content or increased vitamin content. Polypeptides of interest also include, for example, those resulting in or contributing to a reduced content of an unwanted component in a harvested crop, e.g., phytic acid, or sugar degrading enzymes. By “resulting in” or “contributing to” is intended that the polypeptide of interest can directly or indirectly contribute to the existence of a trait of interest (e.g., increasing cellulose degradation by the use of a heterologous cellulase enzyme). Any such polypeptides may be stacked with the BIOA or BIO3-BIO1 enzymes of the invention.

In some embodiments, the polypeptide contributes to improved digestibility for food or feed. Xylanases are hemicellulolytic enzymes that improve the breakdown of plant cell walls, which leads to better utilization of the plant nutrients by an animal. This leads to improved growth rate and feed conversion. Also, the viscosity of the feeds containing xylan can be reduced. Heterologous production of xylanases in plant cells also can facilitate lignocellulosic conversion to fermentable sugars in industrial processing. Numerous xylanases from fungal and bacterial microorganisms have been identified and characterized (see, e.g., U.S. Pat. No. 5,437,992; Coughlin et al. (1993) “Proceedings of the Second TRICEL Symposium on Trichoderma reesei Cellulases and Other Hydrolases” Espoo; Souminen and Reinikainen, eds. (1993) Foundation for Biotechnical and Industrial Fermentation Research 8:125-135; U.S. Patent Publication No. 2005/0208178; and PCT Publication No. WO 03/16654). In particular, three specific xylanases (XYL-I, XYL-II, and XYL-III) have been identified in T. reesei (Tenkanen et al. (1992) Enzyme Microb. Technol. 14:566; Torronen et al. (1992) Bio/Technology 10:1461; and Xu et al. (1998) Appl. Microbiol. Biotechnol. 49:718). Any such polypeptides may be stacked with the BIOA or BIO3-BIO1 enzymes of the invention.

In other embodiments, a polypeptide useful for the disclosure can be a polysaccharide degrading enzyme. Plants of this disclosure producing such an enzyme may be useful for generating, for example, fermentation feedstocks for bioprocessing. In some embodiments, enzymes useful for a fermentation process include alpha amylases, proteases, pullulanases, isoamylases, cellulases, hemicellulases, xylanases, cyclodextrin glycotransferases, lipases, phytases, laccases, oxidases, esterases, cutinases, granular starch hydrolyzing enzyme and other glucoamylases. Polysaccharide-degrading enzymes include: starch degrading enzymes such as α-amylases (EC 3.2.1.1), glucuronidases (E.C. 3.2.1.131); exo-1,4-α-D glucanases such as amyloglucosidases and glucoamylase (EC 3.2.1.3), β-amylases (EC 3.2.1.2), α-glucosidases (EC 3.2.1.20), and other exo-amylases; starch debranching enzymes, such as a) isoamylase (EC 3.2.1.68), pullulanase (EC 3.2.1.41), and the like; b) cellulases such as exo-1,4-3-cellobiohydrolase (EC 3.2.1.91), exo-1,3-β-D-glucanase (EC 3.2.1.39), β-glucosidase (EC 3.2.1.21); c) L-arabinases, such as endo-1,5-α-L-arabinase (EC 3.2.1.99), α-arabinosidases (EC 3.2.1.55) and the like; d) galactanases such as endo-1,4-β-D-galactanase (EC 3.2.1.89), endo-1,3-β-D-galactanase (EC 3.2.1.90), α-galactosidase (EC 3.2.1.22), β-galactosidase (EC 3.2.1.23) and the like; e) mannanases, such as endo-1,4-β-D-mannanase (EC 3.2.1.78), β-mannosidase (EC 3.2.1.25), α-mannosidase (EC 3.2.1.24) and the like; f) xylanases, such as endo-1,4-β-xylanase (EC 3.2.1.8), β-D-xylosidase (EC 3.2.1.37), 1,3-β-D-xylanase, and the like; and g) other enzymes such as α-L-fucosidase (EC 3.2.1.51), α-L-rhamnosidase (EC 3.2.1.40), levanase (EC 3.2.1.65), inulanase (EC 3.2.1.7), and the like. In one embodiment, the α-amylase is the synthetic α-amylase, Amy797E, described is U.S. Pat. No. 8,093,453, herein incorporated by reference in its entirety. Any such polypeptides may be stacked with the BIOA or BIO3-BIO1 enzymes of the invention.

Further enzymes which may be used with the disclosure include proteases, such as fungal and bacterial proteases. Fungal proteases include, but are not limited to, those obtained from Aspergillus, Trichoderma, Mucor and Rhizopus, such as A. niger, A. awamori, A. oryzae and M. miehei. In some embodiments, the polypeptides of this disclosure can be cellobiohydrolase (CBH) enzymes (EC 3.2.1.91). In one embodiment, the cellobiohydrolase enzyme can be CBH1 or CBH2. Any such polypeptides may be stacked with the BIOA or BIO3-BIO1 enzymes of the invention.

Other enzymes useful with the disclosure include, but are not limited to, hemicellulases, such as mannases and arabinofuranosidases (EC 3.2.1.55); ligninases; lipases (e.g., E.C. 3.1.1.3), glucose oxidases, pectinases, xylanases, transglucosidases, alpha 1,6 glucosidases (e.g., E.C. 3.2.1.20); esterases such as ferulic acid esterase (EC 3.1.1.73) and acetyl xylan esterases (EC 3.1.1.72); and cutinases (e.g. E.C. 3.1.1.74). Any such polypeptides may be stacked with the BIOA or BIO3-BIO1 enzymes of the invention.

In other embodiments, the BIOA or BIO3-BIO1 enzymes described herein may be stacked with polynucleotides that encode polypeptides which increase protein content, and/or alter seed composition and/or fatty acid content. Such sequences include, but are not limited to, sequences disclosed in PCT Appl. No. PCT/CN2022/075977 and PCT Appl. No. PCT/CN2022/075982 both filed on Feb. 11, 2022, WO2021/044027; US2020/0131524; and US2021/0403933, each of which is incorporated by reference in its entirety.

In one embodiment, the BIO3-BIO1 and/or BioA enzymes described herein are stacked with a PPO resistance trait. Suitably therefore the plant or part thereof described herein comprises a PPO resistance trait, suitably it further comprises a modified PPO enzyme. In one embodiment therefore, the additional modified enzyme is a modified protoporphyrinogen oxidase (PPO) enzyme.

Suitably therefore the plant or part thereof may further be modified to comprise a PPO enzyme that provides the plant or part thereof with increased resistance to a compound which inhibits a PPO enzyme relative to an unmodified plant. Suitably therefore the plant or part thereof may be modified to comprise a PPO enzyme having one or more modifications which provide the plant or part thereof with a PPO resistance trait, suitably by providing increased resistance to a compound which inhibits a PPO enzyme relative to an unmodified plant. Suitably the plant or part thereof may comprise a recombinant polynucleotide encoding a PPO enzyme having one or more modifications. Suitably the or each modification provides the plant with an increased resistance to compound which inhibits the PPO enzyme relative to an unmodified plant. Suitably the or each modification provides the plant with an increased resistance to a herbicide which inhibits the PPO enzyme. Suitable such modifications to PPO enzymes are described in any of the applications referenced hereinabove.

Suitable such herbicides which may inhibit the PPO enzyme include benzoxazinone derivatives or phenylpyridine derivatives. Suitable such herbicides which may inhibit the PPO enzyme include Tiafenacil, Saflufenacil, Butfenacil, Flumioxazin, Fomesafen, Actifluorfen, Oxyfluorfen, Sulfentrazone, Pentoxazone, Pyraflufen-ethyl, Oxadiozon, Fluthiacet-methyl, Pyraclonil or a combination thereof.

Suitably increased resistance to a compound or herbicide which inhibits a PPO enzyme relative to an unmodified plant is defined in the same way as the increased resistance to the compound/herbicide which inhibits the biotin synthesis pathway elsewhere herein. Suitably the plant or part thereof may be modified to comprise a PPO enzyme in the same manner as it is modified to comprise a BIO3-BIO1 or BioA enzyme described elsewhere herein, suitably by transformation, targeted insertion, molecular stack or breeding stack, or any other method described herein.

Suitably the PPO enzyme may be derived from a plant, fungus, algae or bacterium. In some embodiments, the PPO enzyme is derived from a plant. Suitably the PPO enzyme may be an endogenous or a heterologous enzyme to the plant of the invention. In one embodiment, the PPO enzyme is a heterologous enzyme. Suitably the PPO enzyme may be derived from Arabidopsis thaliana, Amaranthus tuberculatus, Alopecurus myosuroides, Zea mays, Tritucum aestivum, Glycine max, Oryza sativa, Brassica napus, for example. In some embodiments, the PPO enzyme derived from a bacterium. In some embodiments, the PPO enzyme is a HemG or HemY PPO. Suitably the PPO enzyme may be derived from E. coli, Oscillatoria nigro-viridis, Lyngby asp., Halothece sp., Microcoleus vaginatus, Thermosynechococcus elongatus, Synechococcus sp., Thermosynechococcus vulcanus, Xanthomonas campestris, Chitinophaga pinensis, Enterobacter cloacae, Pectobacterium carotovorum, for example.

Suitably the or each modification to the PPO enzyme may be a deletion, insertion, addition or substitution as is described elsewhere herein. Suitably the or each modification to the PPO enzyme is an amino acid substitution. Suitably the or each PPO enzyme, and the or each modification thereto, is selected from any one of those PPO tolerant traits mentioned hereinabove, and any of those described in WO2007/024739, WO2012/080975, WO2013/189984, WO2015/022636, WO2015/022639, WO2015/022640, WO2015/092706, WO2016/099153, WO2016/203307, WO2017/023778, WO2017/039969, WO2017/217793, WO2017/217794, WO2018/019860, WO2018/114759, WO2019/118726, WO2020/251313, which are incorporated herein by reference.

### Breeding Plants

The development or regeneration of transgenic plants containing a nucleic acid molecule that encodes recombinant BIO3-BIO1 and/or BioA of the invention is well known in the art. The regenerated plants may be self-pollinated to provide homozygous transgenic plants, as discussed above. Otherwise, pollen obtained from the regenerated plants is crossed to seed-grown plants of agronomically important lines. Conversely, pollen from plants of these important lines is used to pollinate regenerated plants.

The at least partially resistant plants and progeny of such plants described herein (such as transformed, modified or transgenic plants described herein), can be used in methods for preparing at least partially resistant plants, plants having increased tolerance to compounds which inhibit the biotin synthesis pathway, and seeds of such plants. Thus, for example, the plants exemplified herein may be used in breeding programs to develop additional at least partially herbicide resistant plants, such as commercial varieties of such plants. In accordance with such methods, a first parent plant may be used in crosses with a second parent plant, where at least one of the first or second parent plants contains a BIO3-BIO1 and/or BioA enzyme as described herein. One application of the process is in the production of F1 hybrid plants. Another aspect of this process is that the process can be used for the development of novel parent, dihaploid or inbred lines. For example, a plant line as described herein could be crossed to any second plant, and the resulting hybrid progeny each selfed and/or sibbed for about 5 to 7 or more generations, thereby providing a large number of distinct, parent lines. These parent lines could then be crossed with other lines and the resulting hybrid progeny analysed for beneficial characteristics. In this way, novel lines conferring desirable characteristics could be identified. Various breeding methods may be used in the methods, including haploidy, pedigree breeding, single-seed descent, modified single seed descent, recurrent selection, and backcrossing.

The plants and progeny thereof may display a synergistic effect rather than additive effect of tolerance to compounds which inhibit the biotin synthesis pathway, whereby the level of tolerance in the plants and the progeny thereof comprising multiple mutations is greater than the combined tolerance of plants comprising a single BIO3-BIO1 and/or BioA enzyme.

Plant lines containing the BIO3-BIO1 and/or BioA of the present invention can be crossed by either natural or mechanical techniques. Mechanical pollination can be effected either by controlling the types of pollen that can be transferred onto the stigma or by pollinating by hand.

Any breeding method may be used in the methods of the present invention. In one example, the resistant plants of the present invention may be bred using a haploid method. In such methods, parents having the genetic basis for the desired complement of characteristics are crossed in a simple or complex cross. Crossing (or cross-pollination) refers to the transfer of pollen from one plant to a different plant. Progeny of the cross are grown and microspores (immature pollen grains) are separated and filtered, using techniques known to those skilled in the art [(e.g. Swanson, E. B. et al, (1987) Plant Cell Reports, 6: 94-97, “Efficient isolation of microspores and the production of microspore-derived embryos in Brassica napus, L.; and Swanson, E. B., (1990) Microspore culture in Brassica, pp. 159-169 in Methods in Molecular Biology, vol. 6, Plant Cell and Tissue Culture, Humana Press]. These microspores exhibit segregation of genes. The microspores are cultured in the presence of an appropriate AHAS-inhibitor herbicide, such as imazethapyr (e.g. PURSUIT™) or imazamox (e.g. SOLO™, BEYOND™, and RAPTOR™) or a 50/50 mix of imazethapyr and imazamox (e.g. ODYSSEY™), which kills microspores lacking the mutations responsible for resistance to the herbicide. Microspores carrying the genes responsible for resistance to the herbicide survive and produce embryos, which form haploid plants. Their chromosomes are then doubled to produce doubled haploids.

Other breeding methods may also be used in accordance with the present invention. For example, pedigree breeding may be used for the improvement of largely self-pollinating crops such as Brassica and canola. Pedigree breeding starts with the crossing of two genotypes, each of which may have one or more desirable characteristics that is lacking in the other or which complements the other. If the two original parents do not provide all of the desired characteristics, additional parents can be included in the crossing plan. These parents may be crossed in a simple or complex manner to produce a simple or complex F1. An F2 population is produced from the F1 by selfing one or several F1 plants, or by intercrossing two F1's (i.e., sib mating). Selection of the best individuals may begin in the F2 generation, and beginning in the F3 the best families, and the best individuals within the best families are selected. Replicated testing of families can begin in the F4 generation to improve the effectiveness of selection for traits with low heritability. At an advanced stage of inbreeding (i.e., F6 and F7), the best lines or mixtures of phenotypically similar lines may be tested for potential release as new cultivars. However, the pedigree method is more time-consuming than the haploidy method for developing improved plants which are at least partially resistant to biotin pathway inhibiting compounds, because the plants exhibit segregation for multiple generations, and the recovery of desirable traits is relatively low.

The single seed descent (SSD) procedure may also be used to breed improved varieties. The SSD procedure in the strict sense refers to planting a segregating population, harvesting a sample of one seed per plant, and using the population of single seeds to plant the next generation. When the population has been advanced from the F2 to the desired level of inbreeding, the plants from which lines are derived will each trace to different F2 individuals. The number of plants in a population declines each generation due to failure of some seeds to germinate or some plants to produce at least one seed. As a result, not all of the plants originally sampled in the F2 population will be represented by a progeny when generation advance is completed.

In a multiple-seed procedure, canola breeders commonly harvest one or more pods from each plant in a population and thresh them together to form a bulk. Part of the bulk is used to plant the next generation and part is put in reserve. The procedure has been referred to as modified single-seed descent or the pod-bulk technique. The multiple-seed procedure has been used to save labour at harvest. It is considerably faster to thresh pods with a machine than to remove one seed from each by hand for the single-seed procedure. The multiple-seed procedure also makes it possible to plant the same number of seeds of a population each generation of inbreeding. Enough seeds are harvested to make up for those plants that did not germinate or produce seed.

Backcross breeding can be used to transfer a gene or genes for a simply inherited, highly heritable trait from a source variety or line (the donor parent) into another desirable cultivar or inbred line (the recurrent parent). After the initial cross, individuals possessing the phenotype of the donor parent are selected and are repeatedly crossed (backcrossed) to the recurrent parent. When backcrossing is complete, the resulting plant is expected to have the attributes of the recurrent parent and the desirable trait transferred from the donor parent.

Improved varieties may also be developed through recurrent selection. In this method, genetically variable population of heterozygous individuals is either identified or created by intercrossing several different parents. The best plants are selected based on individual superiority, outstanding progeny, or excellent combining ability. The selected plants are intercrossed to produce a new population in which further cycles of selection are continued.

At least partially resistant plants can be produced by cross-pollinating a first plant with a second plant and allowing the pollen acceptor plant (can be either the first or second plant) to produce seed from this cross pollination. Seeds and progeny plants generated therefrom can have the mutation crossed into the genome of the seed and/or progeny plants. The pollen-acceptor plant can be either the first or second plant. The first plant comprises nucleic acid encoding a BIO3-BIO1 or BioA enzyme as disclosed herein. The second plant can be any compatible plant and may comprise a second same or different BIO3-BIO1 and/or BioA enzyme. The first and second enzymes may comprise a nucleic acid encoding the same or different amino acid substitution(s) or deletions relative to a wild-type BIO3-BIO1 and/or BioA enzyme. Seeds or progeny plants arising from the cross which comprise one or two nucleic acids encoding BIO3-BIO1 and/or BioA enzymes can be selected.

When the first and second plants are homozygous for the first and second nucleic acid molecules, respectively, each of the resulting progeny plants comprises one copy of each of the first and second nucleic acid molecules and the selection step can be omitted. When at least one of the first and second plants is heterozygous, progeny plants comprising both nucleic acid molecules can be selected, for example, by analyzing the DNA of progeny plants to identify progeny plants comprising both the first and second nucleic acid molecules or by testing the progeny plants for increased herbicide tolerance.

Descendent and/or progeny plants may be evaluated for the nucleic acid molecules of the present invention by any method to determine the presence of a specific BIO3-BIO1 and/or BioA nucleic acid or enzyme.

Therefore, also provided herein are methods of selecting a plant or part thereof that includes a BIO3-BIO1 and/or BioA enzyme of the invention, or a nucleic acid or expression vector encoding it, by exposing the plant or part thereof to an effective amount of a compound which inhibits the biotin synthesis pathway sufficient to prevent or reduce the growth of a plant that does not include at least a BIO3-BIO1 and/or BioA enzyme of the invention, or a nucleic acid or expression vector encoding it. It may then be determined by the methods described herein whether the plant has been affected (e.g. has reduced growth or reduced damage) by the compound. Plants that are unaffected by the compound may then be selected.

Methods of determining whether a plant includes the BIO3-BIO1 and/or BioA enzyme of the invention, or a nucleic acid or expression vector encoding it and/or is affected by a compound which inhibits the biotin synthesis pathway include phenotypic evaluations, genotypic evaluations, or combinations thereof. The progeny plants may be evaluated in subsequent generations for resistance to the compound, and other desirable traits. Resistance to compounds which inhibit the biotin synthesis pathway may be evaluated by exposing plants to one or more appropriate compounds and evaluating injury. Some traits, such as lodging resistance and plant height, may be evaluated through visual inspection of the plants, while earliness of maturity may be evaluated by a visual inspection of seeds within pods (siliques). Other traits, such as oil percentage, protein percentage, and total glucosinolates of seeds may be evaluated using techniques such as Near Infrared Spectroscopy and/or liquid chromatography and/or gas chromatography.

Plants of the present invention can also be identified using any genotypic analysis method. Genotypic evaluation of the plants includes using techniques such as Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), Allele-specific PCR (AS-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as “Microsatellites”. Additional compositions and methods for analyzing the genotype of the plants provided herein include those methods disclosed in U.S. Publication No. 2004/0171027, U.S. Publication No. 2005/02080506, and U.S. Publication No. 2005/0283858, the entireties of which are hereby incorporated by reference.

Evaluation and manipulation (through exposure to one or more appropriate compounds which inhibit the biotin synthesis pathway) may occur over several generations. The performance of the new lines may be evaluated using objective criteria in comparison to check varieties. Lines showing the desired combinations of traits are either crossed to another line or self-pollinated to produce seed.

“Sequencing DNA” refers to determining the nucleic acid sequence of a piece of DNA, e.g. of a gene. Standard methods and commercial services are known in the art. Basic methods for DNA sequencing include the Maxam-Gilbert method and the chain termination method. High-throughput techniques have also been developed and may be used in the method of the present invention. These high-throughput techniques include, but are not limited to, Massively parallel signature sequencing (MPSS), Polony sequencing, 454 pyrosequencing, Illumina (Solexa) sequencing, Combinatorial probe anchor synthesis (cPAS), SOLiD sequencing, Ion Torrent semiconductor sequencing, DNA nanoball sequencing, Heliscope single molecule sequencing, Single molecule real time (SMRT) sequencing and Nanopore DNA sequencing.

Sequencing may be carried out using primers that are capable of binding to an isolated polynucleotide of the invention. For example, primers that complimentary to at least a portion of an isolated polynucleotide of the invention.

As used herein, the term “primer” refers to an oligonucleotide which is capable of annealing to a polynucleotide target and serving as a point of initiation of DNA synthesis when placed under conditions in which synthesis of a primer extension product is induced (e.g., in the presence of nucleotides and an agent for polymerization such as DNA polymerase and at a suitable temperature and pH). A primer (in some examples an extension primer and in some examples an amplification primer) may be single stranded for maximum efficiency in extension and/or amplification. The primer may be an oligodeoxyribonucleotide. A primer is typically sufficiently long to prime the synthesis of extension and/or amplification products in the presence of the agent for polymerization. The minimum length of the primer can depend on many factors, including, but not limited to temperature and composition (A/T vs. G/C content) of the primer. In the context of amplification primers, these are typically provided as a pair of bi-directional primers consisting of one forward and one reverse primer or provided as a pair of forward primers as commonly used in the art of DNA amplification such as in PCR amplification.

As such, it will be understood that the term “primer,” as used herein, can refer to more than one primer, particularly in the case where there is some ambiguity in the information regarding the terminal sequence(s) of the target region to be amplified. Hence, a “primer” can include a collection of primer oligonucleotides containing sequences representing the possible variations in the sequence or includes nucleotides which allow a typical base pairing. Primers can be prepared by any suitable method known in the art. Methods for preparing oligonucleotides of specific sequence are known in the art, and include, for example, cloning and restriction of appropriate sequences and direct chemical synthesis. Chemical synthesis methods can include, for example, the phospho di- or tri-ester method, the diethylphosphoramidate method and the solid support method disclosed in U.S. Pat. No. 4,458,066.

Primers can be labelled, if desired, by incorporating detectable moieties by for instance spectroscopic, fluorescence, photochemical, biochemical, immunochemical, or chemical moieties. Primers diagnostic (i.e. able to identify or select based on presence of BIO3-BIO1 or BioA encoding nucleic acids and the BIO3-BIO1 or BioA enzymes thereof as described herein) for resistance to compounds which inhibit the biotin synthesis pathway can be created by any known methods. The PCR method is well described in handbooks and known to the skilled person. After amplification by PCR, target polynucleotides can be detected by hybridization with a probe polynucleotide, which forms a stable hybrid with the target sequence under stringent to moderately stringent hybridization and wash conditions. If it is expected that the probes are essentially completely complementary (i.e., about 99% or greater) to the target sequence, stringent conditions can be used.

If some mismatching is expected, for example if variant polynucleotides are expected with the result that the probe will not be completely complementary, the stringency of hybridization can be reduced. In some examples, conditions are chosen to rule out non-specific/adventitious binding. Conditions that affect hybridization, and that select against non-specific binding are known in the art, and are described in, for example, Sambrook & Russell (2001) Molecular Cloning: A Laboratory Manual, Third Edition, Cold Spring Harbor Laboratory Press, Cold Spring Harbor, New York, United States of America. Generally, lower salt concentration and higher temperature hybridization and/or washes increase the stringency of hybridization conditions.

### Seeds

Also included herein are seeds that are capable of producing a plant or part thereof of the invention. Suitably seeds that comprise a BIO3-BIO1 and/or BioA enzyme, or a polynucleotide or expression vector encoding a BIO3-BIO1 and/or BioA enzyme, which provides increased resistance to a compound which inhibits the biotin synthesis pathway relative to an unmodified plant.

The term “seed” embraces seeds and plant propagules of all kinds including but not limited to true seeds, seed pieces, suckers, corms, bulbs, fruit, tubers, grains, cuttings, cut shoots and the like.

Seeds may be treated or untreated seeds. For example, the seeds can be treated to improve germination, for example, by priming the seeds, or by disinfection to protect against seed-borne pathogens. In another example, seeds can be coated with any available coating to improve, for example, plantability, seed emergence, and protection against seed-borne pathogens. Seed coating can be any form of seed coating including, but not limited to pelleting, film coating, and encrustments.

The seed may be germinated and used to produce or grow a plant or part thereof of the invention. That is a plant or part thereof including a BIO3-BIO1 or BioA enzyme which provides increased resistance to a compound which inhibits the biotin synthesis pathway relative to an unmodified plant.

Also provided herein is a container including seeds of the invention. A container of seeds may contain any number, weight or volume of seeds. For example, a container can contain at least, or greater than, about 10, 25, 50, 75, 100, 200, 300, 400, 500, 600, 700, 800, 900, 1000, 1500, 2000, 2500, 3000, 3500, 4000, 4500, 5000 or more seeds. Alternatively, the container can contain at least, or greater than, about 1 ounce, 5 ounces, 10, ounces, 1 pound, 2 pounds, 3 pounds, 4 pounds, 5 pounds or more seeds.

Containers of plant seeds may be any container available in the art. By way of non-limiting example, a container may be a box, a bag, a packet, a pouch, a tape roll, a pail, a foil, or a tube.

Seeds contained in a containers may be treated or untreated seeds. For example, the seeds can be treated to improve germination, for example, by priming the seeds, or by disinfection to protect against seed-borne pathogens. In another example, seeds can be coated with any available coating to improve, for example, plantability, seed emergence, and protection against seed-borne pathogens. Seed coating can be any form of seed coating including, but not limited to pelleting, film coating, and encrustments.

At least 10% of seeds within a container may be seeds of the invention. For example, at least 10%, 20%, 30%, 40%, 50%, 60%, 70%, 80%, 90%, 95%, 99% or 100% of the seeds in the container may be seeds of the invention.

The seeds of the invention may be hybrid seeds produced by a method including crossing a first plant according to the invention, with a second plant; and obtaining seeds. For example, crossing a plant including BIO3-BIO1 and/or BioA enzyme, or a polynucleotide or expression vector encoding such a BIO3-BIO1 and/or BioA enzyme, which provides increased resistance to a compound which inhibits biotin synthesis with another plant.

The term “hybrid seed” refers to a seed produced by cross-pollinating two plants. Plants grown from hybrid seeds may have improved agricultural characteristics, such as better yield, greater uniformity, and/or disease resistance. Hybrid seeds do not breed true, i.e., the seed produced by self-fertilizing a hybrid plant (the plant grown from a hybrid seed) does not reliably result the next generation in an identical hybrid plant. Therefore, new hybrid seeds must be produced from the parent plant lines for each planting. Since most crop plants have both male and female organs, hybrid seeds can only be produced by preventing self-pollination of the female parent and allowing or facilitating pollination with the desired pollen. There are a variety of methods to prevent self-pollination of the female parent, one method by which self-pollination is prevented is mechanical removal of the pollen producing organ before pollen shed. Commercial hybrid maize seed (maize, Zea mays) production typically involves planting the desired male and female parental lines, usually in separate rows or blocks in an isolated field, treating the female parent plant to prevent pollen shed, ensuring pollination of the female by only the designated male parent, and harvesting hybrid seed from only the female parent. Hybrid seeds may be the result of a single cross (e.g., a first generation cross between two inbred lines), a modified single cross (e.g., a first generation cross between two inbred lines, one or other of which may have been modified slightly by the use of closely related crossing), a double cross (e.g., a first generation of a cross between two single crosses), a three-way cross (e.g., a first generation of a cross between a single cross and an inbred line), a top cross (e.g., the first generation of a cross between an inbred line and an open-pollinated variety, or the first generation of a cross between a single-cross and an open-pollinated variety), or an open pollinated variety (e.g., a population of plants selected to a standard which may show variation but has characteristics by which a variety can be differentiated from other varieties).

As used herein, the terms “cross” or “crossed” refer to the fusion of gametes via pollination to produce progeny (e.g., cells, seeds or plants). The term encompasses both sexual crosses (the pollination of one plant by another) and selfing (self-pollination, e.g., when the pollen and ovule are from the same plant). The term “crossing” refers to the act of fusing gametes via pollination to produce progeny.

### Plants or Parts Thereof

The present invention may be for use with any plant species and the progeny thereof, including, but not limited to, monocots and dicots.

Examples of plant species of interest include, but are not limited to, corn or maize (Zea mays), Brassica sp. (e.g., B. napus, B. rapa, B. juncea), including those Brassica species useful as sources of seed oil, alfalfa (Medicago sativa), rice (Oryza sativa), rye (Secale cereale), sorghum (Sorghum bicolor, Sorghum vulgare), millet (e.g., pearl millet (Pennisetum glaucum), proso millet (Panicum miliaceum), foxtail millet (Setaria italica), finger millet (Eleusine coracana)), sunflower (Helianthus annuus), safflower (Carthamus tinctorius), wheat (Triticum aestivum, T. Turgidum ssp. durum), soybean (Glycine max), tobacco (Nicotiana tabacum), potato (Solarium tuberosum), peanuts (Arachis hypogaea), cotton (Gossypium barbadense, Gossypium hirsutum), sweet potato (Ipomoea batatus), cassava (Manihot esculenta), coffee (Coffea spp.), coconut (Cocos nucifera), pineapple (Ananas comosus), citrus trees (Citrus spp.), cocoa (Theobroma cacao), tea (Camellia sinensis), banana (Musa spp.), avocado (Persea americana), fig (Ficus casica), guava (Psidium guajava), mango (Mangifera indica), olive (Olea europaea), papaya (Carica papaya), cashew (Anacardium occidentale), macadamia (Macadamia integrifolia), almond (Primus amygdalus), sugar beets (Beta vulgaris), sugarcane (Saccharum spp.), oats (Avena sativa), barley (Hordeum vulgare), Gemmiferous Spikemoss (Selaginella moellendorffii) vegetables, ornamentals, and conifers.

In one embodiment, plants of the present invention are crop plants (for example, sunflower, Brassica sp., cotton, sugar, beet, soybean, peanut, alfalfa, safflower, tobacco, corn, rice, wheat, rye, barley triticale, sorghum, millet, etc.).

In another embodiment, plants of the present invention may also include various types of cover crop plants. Exemplary cover crop plants include, but are not limited to, Brassica sp. (e.g., B. carinata, B. napus, B. rapa, B. hirta, B. juncea, B. nigra), radish (Raphanus sativus), Camelina sp. (e.g., C. sativa), pennycress (Thlaspi arvense), clover (Trifolium sp., e.g., T. incarnatum, T. pratense, T. repens, T. subterraneum), field peas (Pisum sativum), Vicia sp. (e.g., V. villosa, V. lutea, V. nigricans, V. sativa), rye (Secale cereale), barley (Hordeum vulgare), winter wheat (Triticum aestivum), oats (Avena sativa), annual ryegrass (Lolium multiflorum), buckwheat (Fagopyrum esculentum), Sinapsis a/ba, alfalfa (Medicago sativa)

As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

As used herein unless clearly indicated otherwise, the term “plant” is intended to mean a plant at any developmental stage, as well as any part or parts of a plant that may be attached to or separate from a whole intact plant. Such parts of a plant include, but are not limited to, organs, tissues, and cells of a plant including, plant calli, plant clumps, plant protoplasts and plant cell tissue cultures from which plants can be regenerated. Examples of particular plant parts include a stem, a leaf, a root, an inflorescence, a flower, a floret, a fruit, a pedicle, a peduncle, a stamen, an anther, a stigma, a style, an ovary, a petal, a sepal, a carpel, a root tip, a root cap, a root hair, a leaf hair, a seed hair, a pollen grain, a microspore, an embryos, an ovule, a cotyledon, a hypocotyl, an epicotyl, xylem, phloem, parenchyma, endosperm, a companion cell, a guard cell, and any other known organs, tissues, and cells of a plant. Furthermore, it is recognized that a seed is a plant part.

A “plant cell” is a structural and physiological unit of a plant, comprising a protoplast and a cell wall. The plant cell may be in the form of an isolated single cell or a cultured cell, or as a part of a higher organized unit such as, for example, plant tissue, a plant organ, or a whole plant. A “plant part” is a distinct and visibly structured and differentiated part of a plant such as a root, stem, leaf, flower bud, or embryo.

Further provided are genomes having stably integrated a polynucleotide encoding at least one of the BIO3-BIO1 and/or BioA enzymes provided herein.

The plants, progeny thereof or parts thereof of the invention express at least one of a BIO3-BIO1 and/or BioA enzyme. Expression of the enzymes, polynucleotides encoding said enzymes, or expression vectors of the invention provides a plant that is at least partially resistant to compounds which inhibit the biotin synthetic pathway, such as those herbicides described herein. For example, the plants, progeny thereof or parts thereof of the invention have increased resistance to a herbicide which inhibits the biotin synthesis pathway. The increase in resistance may be determined by comparison to a wild-type or control plant as described herein. For example, a plant that has not been modified to include or express the BIO3-BIO1, BioA enzymes, polynucleotides encoding them, or expression vectors of the invention.

### Methods of Producing Modified Plants or Increasing Resistance of a Plant

Provided herein are methods of conferring increased resistance to compounds which inhibit the biotin synthesis pathway to a plant or part thereof by modifying the plant to comprise a BIO3-BIO1 and/or BioA enzyme which provides said resistance. Suitably such methods may include (i) a method of producing a modified plant or part thereof having an increased resistance to a compound which inhibits the biotin synthesis pathway, and (ii) a method of increasing the resistance of a plant or part thereof to a compound which inhibits the biotin synthesis pathway.

Suitably either method comprises a step of modifying the plant or part thereof to comprise a BIO3-BIO1 and/or BioA enzyme that provides the increased resistance.

Suitably modifying the plant may comprise increasing the expression of, or overexpressing, a BIO3-BIO1 and/or BioA enzyme in the plant or part thereof, suitably wherein the increased expression provides the increased resistance. Alternatively, or additionally, modifying the plant may comprise providing the plant or part thereof with a BIO3-BIO1 and/or BioA enzyme having one or more modifications, wherein expression of said enzyme in the plant provides the increased resistance. Suitably, expression of said enzyme in the plant provides the increased resistance.

Suitably wherein the increased resistance may be caused by or conferred by one or more of the modifications to the BIO3-BIO1 and/or BioA enzyme.

Suitably such steps may comprise providing the plant or part thereof with a recombinant polynucleotide encoding a BIO3-BIO1 and/or a BioA enzyme. Suitably the recombinant polynucleotide, may encode a wild type, unmodified BIO3-BIO1 enzyme and/or BioA enzyme, or may encode a modified BIO3-BIO1 and/or BioA enzyme as described above.

Suitably providing may comprise introducing the recombinant polynucleotide encoding a BIO3-BIO1 and/or a BioA enzyme into the plant or part thereof, or introducing the BIO3-BIO1 and/or BioA protein into the plant or part thereof. Suitably the BIO3-BIO1 or BioA enzyme may be introduced into a plant or part thereof by introducing a polynucleotide of the invention which encodes a BIO3-BIO1 and/or BioA enzyme. Thus, plants of the invention may be referred to as modified or transgenic plants.

Suitably introducing the BIO3-BIO1 and/or BioA enzyme into a plant or part thereof may be carried out by transforming the plant or part thereof with a recombinant polynucleotide encoding a BIO3-BIO1 and/or a BioA enzyme, which may be a modified or unmodified BIO3-BIO1 and/or a BioA enzyme.

Suitably the recombinant polynucleotide may further encode a transit peptide, such as a mitochondrial transit peptide. Suitably as a fusion with the BIO3-BIO1 and/or a BioA enzyme. Suitably therefore the recombinant polynucleotide may comprise a chimeric polynucleotide encoding a wild type or modified BIO3-BIO1 and/or BioA as described above and a transit peptide operably linked thereto, suitably a mitochondrial transit peptide operably linked thereto. Suitable transit peptides are described elsewhere herein.

Suitably the recombinant polynucleotide may be part of an expression construct, or comprised on an expression vector. Suitably the expression construct or vector may comprise one or more expression elements such as a promoter, as is described elsewhere herein. Suitably therefore the methods may comprise providing, introducing or transforming the plant or part thereof with an expression construct or expression vector comprising a polynucleotide encoding a BIO3-BIO1 and/or BioA enzyme, suitably which may be a recombinant polynucleotide

Suitably the methods may further comprise a step of expressing the recombinant polynucleotide to produce the BIO3-BIO1 and/or BioA enzyme in the plant or part thereof. Suitably the expression may be constitutive, suitably therefore the polynucleotide encoding the BIO3-BIO1 and/or BioA enzyme may be under the control of a constitutive promoter. Alternatively, expression of the polynucleotide may comprise inducing expression thereof, suitably by contacting the plant or part thereof with an inducer. Suitably in such embodiments the polynucleotide encoding the BIO3-BIO1 and/or BioA enzyme may be under the control of an inducible promoter, suitably therefore the expression construct or vector may comprise an inducible promoter operably linked to the polynucleotide encoding the BIO3-BIO1 and/or BioA enzyme. Suitable inducible promoters and inducers are well known in the art.

In addition to or alternatively a plant may be modified by in situ editing of the endogenous genetic material in order to provide a gene that expresses a BIO3-BIO1 enzyme which provides increased resistance to compounds which inhibit the biotin synthesis pathway.

Suitably in such embodiments, a plant may be provided with the components of a gene editing system for modifying an endogenous gene sequence of the plant encoding BIO3-BIO1 enzyme at one or more positions to produce a modified gene sequence encoding a BIO3-BIO1 enzyme which provides increased resistance to a compound which inhibits the biotin synthesis pathway. Suitably the plant may be transformed with one or more polynucleotides encoding a gene editing system for modifying an endogenous gene sequence of the plant encoding BIO3-BIO1 enzyme at one or more positions to produce a modified gene sequence encoding a BIO3-BIO1 enzyme which provides increased resistance to a compound which inhibits the biotin synthesis pathway. Thus providing a plant or part thereof that expresses a BIO3-BIO1 enzyme which provides increased resistance to a compound which inhibits the biotin synthesis pathway.

An endogenous BIO3-BIO1 encoding gene sequence may be edited in situ by way of gene editing techniques in order to provide a modified BIO3-BIO1 enzyme that is at least partially resistant and/or provides increased resistance to a to compound that inhibits the biotin synthesis pathway, such as those described herein and as such a modified plant as described herein. Such genome editing and/or mutagenesis technologies are well known in the art. As well, introduction may be accomplished by any manner known in the art, including: introgression, transgenic, cisgenic, or site-directed nucleases (SDN). Particularly, the modification to the gene sequence is introduced by way of site-directed nuclease (SDN). More particularly, the SDN is selected from: meganuclease, zinc finger, transcription activator-like effector nucleases system (TALEN) or Clustered Regularly Interspaced Short Palindromic Repeats system (CRISPR) system. SDN is also referred to as “genome editing”, or genome editing with engineered nucleases (GEEN). This is a type of genetic engineering in which DNA is inserted, deleted or replaced in the genome of an organism using engineered nucleases that create site-specific double-strand breaks (DSBs) at desired locations in the genome. The induced double-strand breaks are repaired through nonhomologous end-joining (NHEJ) or homologous recombination (HR), resulting in targeted mutations (‘edits’). Particularly SDN may comprises techniques such as: Meganucleases, Zinc finger nucleases (ZFNs), Transcription Activator-Like Effector-based Nucleases (TALEN) (Feng et al. 2013 Cell Res. 23, 1229-1232, Sander & Joung Nat. Biotechnol. 32, 347-355 2014), and the Clustered Regularly Interspaced Short Palindromic Repeats (CRISPR-Cas) system. Gene editing may also be achieved by SDN-2. SDN-2 is similar to SDN, but also provides a small nucleotide template complementary to the area of the break. The template contains one or more sequences modifications to the genomic DNA which are incorporated to create the mutation to the target gene. In one embodiment, the gene editing system may include a CRISPR-Cas system.

As used herein, the term “guide RNA” or “gRNA” generally refers to an RNA molecule (or a group of RNA molecules collectively) that can bind to a CRISPR system effector, such as a Cas or a Cpf 1 protein, and aid in targeting the Cas or Cpfl protein to a specific location within a target polynucleotide (e.g., a DNA). A guide RNA of the invention can be an engineered, single RNA molecule (sgRNA), where for example the sgRNA comprises a crRNA segment and optionally a tracrRNA segment. A guide RNA of the invention can also be a dual-guide system, where the crRNA and tracrRNA molecules are physically distinct molecules which then interact to form a duplex for recruitment of a CRISPR system effector, such as Cas9, and for targeting of that protein to the target polynucleotide.

As used herein, the term “crRNA” or “crRNA segment” refers to an RNA molecule or to a portion of an RNA molecule that includes a polynucleotide targeting guide sequence, a stem sequence involved in protein-binding, and, optionally, a 3′-overhang sequence. The polynucleotide targeting guide sequence is a nucleic acid sequence that is complementary to a sequence in a target DNA (for example a gene encoding a BIO3-BIO1 enzyme). This polynucleotide targeting guide sequence is also referred to as the “protospacer”. In other words, the polynucleotide targeting guide sequence of a crRNA molecule interacts with a target DNA in a sequence-specific manner via hybridization (i.e., base pairing). As such, the nucleotide sequence of the polynucleotide targeting guide sequence of the crRNA molecule may vary and determines the location within the target DNA that the guide RNA and the target DNA will interact.

The polynucleotide targeting guide sequence of a crRNA molecule can be modified (e.g., by genetic engineering) to hybridize to any desired sequence within a target DNA. The polynucleotide targeting guide sequence of a crRNA molecule of the invention can have a length from about 12 nucleotides to about 100 nucleotides. For example, the polynucleotide targeting guide sequence of a crRNA can have a length of from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the polynucleotide targeting guide sequence of a crRNA can have a length of from about 17 nt to about 27 nts.

For example, the polynucleotide targeting guide sequence of a crRNA can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. The nucleotide sequence of the polynucleotide targeting guide sequence of a crRNA can have a length at least about 12 nt. In some embodiments, the polynucleotide targeting guide sequence of a crRNA is 20 nucleotides in length. In some embodiments, the polynucleotide targeting guide sequence of a crRNA is 19 nucleotides in length.

The present invention also provides a guide RNA comprising an engineered crRNA, wherein the crRNA comprises a bait RNA segment capable of hybridizing to a genomic target sequence. This engineered crRNA may be a physically distinct molecule, as in a dual-guide system.

As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The present invention also provides a guide RNA comprising an engineered tracrRNA, wherein the tracrRNA further comprises a bait RNA segment that is capable of binding to a donor DNA molecule. The engineered tracrRNA may be a physically distinct molecule, as in a dual-guide system, or may be a segment of a sgRNA molecule.

The guide RNA, either as a sgRNA or as two or more RNA molecules, does not contain a tracrRNA, as it is known in the art that some CRISPR-associated nucleases, such as Cpfl (also known as Casl2a), do not require a tracrRNA for its RNA-mediated endonuclease activity (Qi et al., (2013), Cell, 152: 1173-1183; Zetsche et al., (2015), Cell 163: 759-771). Such a guide RNA of the invention may comprise a crRNA with the bait RNA operably linked at the 5′ or 3′ end of the crRNA. Cpfl also has RNase activity on its cognate pre-crRNA (Fonfara et al., (2016), Nature, doi.org/10.1038/naturel7945).

A guide RNA of the invention may comprise multiple crRNAs which the Cpfl possesses to mature crRNAs. Each of these crRNAs may be operably linked to a bait RNA. At least one of these crRNAs may be operably linked to a bait RNA. The bait RNA may be specific to a sequence of interest (SOI), or it may be a “universal” bait, which has a corresponding “universal” prey sequence on the donor DNA molecule.

The present invention also provides a polynucleotide comprising a sequence encoding a guide RNA of the invention. The polynucleotide may be a DNA or an RNA molecule. The polynucleotide molecule may be circularized or linear. The polynucleotide may be single stranded, partially double-stranded, or double-stranded. The polynucleotide may be complexed with at least one polypeptide. The polypeptide may have a nucleic acid recognition or nucleic acid binding domain. The polypeptide may be a shuttle for mediating delivery of, for example, a polynucleotide of the invention, a nuclease, and optionally a donor molecule. The polypeptide may be a Feldan Shuttle (U.S. Patent Publication No. 20160298078, herein incorporated by reference). The polynucleotide may comprise an expression cassette capable of driving the expression of the polynucleotide. The polynucleotide may further comprise additional expression cassettes, capable of expressing, for example, a nuclease such as a CRISPR-associated nuclease.

Suitably therefore the plant or part thereof may be provided with, specifically transformed with, a Cas enzyme, or one or more polynucleotides encoding a Cas enzyme, and a polynucleotide sequence encoding a guide RNA. Suitably the guide RNA is complementary to a BIO3-BIO1 gene or regulatory sequences thereof, in the plant or part thereof. Suitably the guide RNA is operable to target the Cas enzyme to edit the BIO3-BIO1 gene and provide the plant or part thereof with increased resistance to a compound which inhibits the biotin synthesis pathway relative to an unmodified plant. A suitable example of such a polynucleotide and of gene editing is shown in example 9. Suitably therefore the plant or part thereof may be provided with, specifically transformed with, a polynucleotide sequence according to any one of SEQ ID NO: 312, 313, or 314.

In addition to or alternatively a plant may be modified by providing the plant or part thereof with one or more regulatory RNA sequences operable to target a gene that encodes a BIO3-BIO1 enzyme or a regulatory sequence thereof. Suitably the plant or part thereof may be transformed with one or more regulatory RNA sequences operable to target a gene that encodes a BIO3-BIO1 enzyme or a regulatory sequence thereof. Suitably the regulatory RNA sequence may be complementary to, and bind to, a gene that encodes a BIO3-BIO1 enzyme or a regulatory sequence thereof in the plant or part thereof, and act to provide increased resistance to compounds which inhibit the biotin synthesis pathway. Suitably the regulatory RNA sequence may provide increased resistance to compounds which inhibit the biotin synthesis pathway by increasing expression of a gene that encodes a BIO3-BIO1 enzyme in the plant or part thereof, or a regulatory sequence which controls expression of a BIO3-BIO1 gene such as an enhancer or promoter. Alternatively, the regulatory RNA sequence may provide increased resistance to compounds which inhibit the biotin synthesis pathway by inhibiting the expression of a regulatory sequence which controls expression of the BIO3-BIO1 gene, such as a repressor. Suitable regulatory RNA sequences may be miRNA, lncRNA, siRNA etc.

“Transformation” refers to a process of introducing an exogenous nucleic acid molecule (for example, a recombinant polynucleotide) into a cell or protoplast and that exogenous nucleic acid molecule is incorporated into a host cell genome or an organelle genome (for example, chloroplast or mitochondria) or is capable of autonomous replication. “Transformed” refers to a cell, tissue, organ, or organism into which a foreign nucleic acid, such as an expression vector or recombinant nucleic acid molecule has been introduced. The nucleic acid molecule can be stably integrated into the genome of the host or the nucleic acid molecule can also be present as an extrachromosomal molecule or transiently expressed. Such an extrachromosomal molecule can be auto-replicating. The nucleic acid molecule can also be introduced into the genome of the chloroplast or the mitochondria of a plant cell.

Methods of transformation of plant cells or tissues include, but are not limited to Agrobacterium mediated transformation method and the Biolistics or particle-gun mediated transformation method. Suitable plant transformation vectors for the purpose of Agrobacterium mediated transformation include-those elements derived from a tumor inducing (Ti) plasmid of Agrobacterium tumefaciens, for example, right border (RB) regions and left border (LB) regions, and others disclosed by Herrera-Estrella et al., Nature 303:209 (1983); Bevan, Nucleic Acids Res. 12:8711-8721 (1984); Klee et al., Bio-Technology 3(7):637-642 (1985). In addition to plant transformation vectors derived from the Ti or root-inducing (Ri) plasmids of Agrobacterium, alternative methods can be used to insert the nucleic acid molecules of this invention into plant cells. Such methods may involve, but are not limited to, for example, the use of liposomes, electroporation, chemicals that increase free DNA uptake, free DNA delivery via microprojectile bombardment, and transformation using viruses or pollen.

A modified cell or plant as provided herein also includes progeny of the cell or plant and progeny produced from a breeding program employing such a modified plant as a parent in a cross and exhibiting an altered phenotype resulting from the presence of the foreign nucleic acid molecule.

The modified cell or plant may be homozygous for the polynucleotide encoding a BIO3-BIO1 and/or BioA enzyme described herein (i.e. those that contain two added genes encoding the enzyme at the same position on each chromosome of the chromosome pair). Homozygous plants may be obtained by crossing (self-pollinating) independent plant isolates containing a single added gene, germinating some of the resulting seeds, and transforming the resulting plant with the target gene.

### Nucleic Acids

The invention further provides isolated polynucleotides that encode a modified BIO3-BIO1 and/or BioA enzyme or fragment thereof as defined in any aspect or embodiment herein. As such, also provided are modified BIO3-BIO1 and/or BioA enzymes or functional fragments thereof that may be expressed from such isolated polynucleotides.

Suitably a polynucleotide encoding a BIO3-BIO1 enzyme may comprise a sequence according to SEQ ID NO: 156 to 158, or a sequence having at least 30%, at least 35%, at least 40%, at least 45%, at least 50%, at least 55%, at least 60%, at least 70%, at least 75%, at least 80%, at least 85%, at least 90%, at least 95%, at least 96%, at least 97%, at least 98%, at least 99% identity thereto. Suitably the polynucleotide sequence according to any of SEQ ID NO: 156 to 158 may be modified to encode a modified BIO3-BIO1 enzyme as described above herein. Suitably such polynucleotides may be comprised in an expression construct or on an expression vector as described herein.

Suitably a polynucleotide encoding a BioA enzyme may comprise a sequence according to SEQ ID NO: 202 or 203, or a sequence having at least 30%, at least 35%, at least 40%, at least 45%, at least 50%, at least 55%, at least 60%, at least 70%, at least 75%, at least 80%, at least 85%, at least 90%, at least 95%, at least 96%, at least 97%, at least 98%, at least 99% identity thereto. Suitably the polynucleotide sequence according to any of SEQ ID NO: 202 to 203 may be modified to encode a modified BioA enzyme as described above herein. Suitably such polynucleotides may be comprised in an expression construct or on an expression vector as described herein.

An “isolated” polynucleotide is substantially separated away from other polynucleotide sequences with which the polynucleotide is normally associated, such as, from the chromosomal or extrachromosomal DNA of a cell in which the polynucleotide naturally occurs. A polynucleotide may be an isolated polynucleotide when it comprises a transgene or part of a transgene present in the genome of another organism. The term also embraces polynucleotides that are biochemically purified so as to substantially remove contaminating polynucleotides and other cellular components. Isolated polynucleotides are substantially free of sequences (such as protein encoding sequences) that naturally flank the nucleic acid (i.e., sequences located at the 5′ and 3′ ends of the polynucleotide) in the genomic DNA of the organism from which the polynucleotide is derived. For example, the isolated polynucleotide can contain less than about 5 kb, 4 kb, 3 kb, 2 kb, 1 kb, 0.5 kb, or 0.1 kb of nucleotide sequences that naturally flank the polynucleotide in genomic DNA of the cell from which the polynucleotide is derived. The isolated polynucleotide may be flanked by its native genomic sequences that control its expression in the cell, for example, the native promoter, or native 3′ untranslated region.

An “isolated polypeptide” as used herein refers to a protein which is free of at least some proteins with which it would normally be found, is essentially free of other proteins from the same source, e.g., from the same cell or species, has been separated from at least about 50 percent of polynucleotides, lipids, carbohydrates, or other materials with which it is naturally found when isolated from the source cell, is not linked (by covalent or noncovalent interaction) to all or a portion of a polypeptide to which the “isolated polypeptide” is linked in nature,. Preferably, the isolated protein is substantially free from other contaminating proteins or polypeptides or other contaminants that are found in its natural environment.

### Expression Constructs and Vectors

The expression constructs and vectors, which include at least one polynucleotide of the present invention inserted therein may be any construct or vector capable of delivering the polynucleotide into a host or host cell and allowing expression of the polynucleotide to provide a functional BIO3-BIO1 and/or BioA enzyme as described herein or fragment thereof.

Such constructs or vectors may contain heterologous polynucleotide sequences, that is polynucleotide sequences that are not naturally found adjacent to polynucleotides of the present invention and that may be derived from a species other than the species from which the polynucleotide molecule(s) are derived. The construct or vector can be either RNA or DNA, either prokaryotic or eukaryotic, and typically the vector is a virus or a plasmid.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. For example the vector may be pBIN 19 (Bevan, Nucl. Acids Res. (1984)).

The expression vector of the invention may include one or more regulatory sequences. For example, the expression vectors can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal. Such a portion of an expression vector may be referred to as an expression construct The expression construct may include one or more regulatory sequences that are functional in plants. Thus allowing expression of the polynucleotide encoding an BIO3-BIO1 and/or BioA enzyme of the invention in a plant.

“Expression construct” as used herein means a nucleic acid sequence capable of directing expression of a particular nucleic acid sequence in an appropriate host cell, comprising a promoter operably linked to the polynucleotide of interest which is operably linked to termination signal sequences. It also typically comprises sequences required for proper translation of the polynucleotide sequence. The expression construct comprising the polynucleotide of interest may be chimeric, meaning that at least one of its components is heterologous with respect to at least one of its other components. The expression construct may also be one that is naturally occurring but has been obtained in a recombinant form useful for heterologous expression. In one embodiment, the expression construct is heterologous with respect to the host, i.e., the particular polynucleotide of the expression cassette does not occur naturally in the host cell and must have been introduced into the host cell or an ancestor of the host cell by a transformation event. The expression of the polynucleotide sequence in the expression construct may be under the control of, for example, a constitutive promoter or of an inducible promoter that initiates transcription only when the host cell is exposed to some particular external stimulus. In the case of a multicellular organism, such as a plant, the promoter can also be specific to a particular tissue, or organ, or stage of development.

The term “regulatory element” or “regulatory sequence” as used herein refers to a nucleic acid that is capable of regulating the transcription and/or translation of an operably linked polynucleotide. Regulatory elements include, but are not limited to, promoters, enhancers, introns, 5′ UTRs, and 3′ UTRs.

Expression cassettes may include in the 5′- 3′ direction of transcription, a transcriptional and translational initiation region (e.g., a promoter), a polynucleotide sequence encoding BIO3-BIO1 and/or BioA of the invention, and a transcriptional and translational termination region (e.g., termination region) functional in plants.

Any promoter can be used in the production of the expression construct and vectors including such expression constructs as described herein. The promoter may be native or analogous, or foreign or heterologous, to the plant host and/or to the polynucleotide sequences encoding BIO3-BIO1 and/or BioA of the invention. Additionally, the promoter may be a natural sequence or alternatively a synthetic sequence. Where the promoter is “foreign” or “heterologous” to the plant host, it is intended that the promoter is not found in the native plant into which the promoter is introduced. Where the promoter is “foreign” or “heterologous” to the polynucleotide encoding BIO3-BIO1 and/or BioA of the invention, it is intended that the promoter is not the native or naturally occurring promoter for the operably linked polynucleotide of the invention.

While it may be preferable to express the polynucleotide encoding BIO3-BIO1 and/or BioA of the invention using heterologous promoters, the native promoter sequences may be used in the preparation of the expression constructs. Such expression constructs may change expression levels of the BIO3-BIO1 and/or BioA enzyme in the plant or plant cell. Thus, the phenotype of the plant or plant cell is altered.

Any promoter can be used in the preparation of expression constructs to control the expression of the polynucleotide encoding BIO3-BIO1 and/or BioA, such as promoters providing for constitutive, tissue-preferred, inducible, or other promoters for expression in plants. Constitutive promoters include, for example, the core promoter of the Rsyn7 promoter and other constitutive promoters disclosed in WO 99/43 838 and U.S. Pat. No. 6,072,050; the core CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); MAS (Velten et al. (1984) EMBO J. 3:2723-2730); ALS promoter (U.S. Pat. No. 5,659,026), and the like. Other constitutive promoters include, for example, U.S. Pat. Nos. 5,608,149; 5,608,144; 5,604,121; 5,569,597; 5,466,785; 5,399,680; 5,268,463; 5,608,142; and 6,177,611.

Tissue-preferred promoters can be utilized to direct expression of the BIO3-BIO1 and/or BioA enzymes of the invention within a particular plant tissue. Such tissue-preferred promoters include, but are not limited to, leaf-preferred promoters, root-preferred promoters, seed-preferred promoters, and stem-preferred promoters. Tissue-preferred promoters include those described in Yamamoto et al. (1997) Plant J. 12(2):255-265; Kawamata et al. (1997) Plant Cell Physiol. 38(7):792-803; Hansen et al. (1997) Mol Gen Genet. 254(3):337-343; Russell et al. (1997) Transgenic Res. 6(2): 157-168; Rinehart et al. (1996) Plant Physiol. 1 12(3):1331-1341; Van Camp et al. (1996) Plant Physiol. 1 12(2):525-535; Canevascini et al. (1996) Plant Physiol. 112(2): 513-524; Yamamoto et al. (1994) Plant Cell Physiol. 35(5):773-778; Lam (1994) Results Probl. Cell Differ. 20:181-196; Orozco et al (1993) Plant Mol Biol. 23(6):1129-1138; Matsuoka et al. (1993) Proc Natl. Acad. Sci. USA 90(20):9586-9590; and Guevara-Garcia et al. (1993) Plant J. 4(3):495-505.

The expression constructs may also comprise transcription termination regions. Where transcription terminations regions are used, any termination region may be used in the preparation of the expression cassettes. For example, the termination region may be native to the transcriptional initiation region, may be native to the operably linked polynucleotide of interest, may be native to the plant host, or may be derived from another source (i.e., foreign or heterologous to the promoter, the polynucleotide of interest encoding BIO3-BIO1 and/or BioA, the plant host, or any combination thereof). Examples of termination regions that are available for use in the expression constructs and vectors of the present invention include those from the Ti-plasmid of A. tumefaciens, such as the octopine synthase and nopaline synthase termination regions. See also Guerineau et al. (1991) Mol. Gen. Genet. 262: 141-144; Sanfacon et al. (1991) Genes Dev. 5:141-149; Mogen et al. (1990) Plant Cell 2:1261-1272; Munroe et al. (1990) Gene 10 91:151-158; Ballas et al. (1989) Nucleic Acids Res. 17:7891-7903; and Joshi et al. (1987) Nucleic Acid Res. 15:9627-9639.

For example, the expression construct may comprise a Tomato Mosaic Virus (TMV) omega 5′ leader and a BIO3-BIO1 and/or BioA encoding gene of interest is excised using Xhol/Kpnl and cloned into pBIN 19 behind a double enhanced 35S promoter and ahead of a NOS 3′ transcription terminator. A suitable exemplary vector comprising such an expression construct is provided herein as SEQ ID NO: 206.

The polynucleotides may be optimized for increased expression in a transformed plant. That is, the polynucleotides encoding the BIO3-BIO1 and/or BioA enzymes can be synthesized using plant-preferred codons for improved expression. See, for example, Campbell and Gowri (1990) Plant Physiol. 92:1-11 for a discussion of host-preferred codon usage. Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

In addition, other sequence modifications can be made to the polynucleotides of the invention. For example, additional sequence modifications that are known to enhance gene expression in a cellular host. These include elimination of sequences encoding spurious polyadenylation signals, exon/intron splice site signals, transposon-like repeats, and other such well-characterized sequences that may be deleterious to gene expression. The G-C content of the sequence may also be adjusted to levels average for a target cellular host, as calculated by reference to known genes expressed in the host cell. In addition, the sequence can be modified to avoid predicted hairpin secondary mRNA structures.

Other polynucleotide sequences may also be used in the preparation of the expression constructs of the present invention, for example to enhance the expression of the BIO3-BIO1 and/or BioA encoding polynucleotide sequence. Such polynucleotide sequences include the introns of the maize Adhl, intron I gene (Callis et al. (1987) Genes and Development 1:1183-1200), and leader sequences, (W-sequence) from the Tobacco Mosaic virus (TMV), Maize Chlorotic Mottle Virus and Alfalfa Mosaic Virus (Gallie et al (1987) Nucleic Acid Res. 15:8693-8711, and Skuzeski et al. (1990) Plant Mol. Biol. 15:65-79, 1990). The first intron from the shrunken-1 locus of maize has been shown to increase expression of genes in chimeric gene constructs. U.S. Pat. Nos. 5,424,412 and 5,593,874 disclose the use of specific introns in gene expression constructs, and Gallie et al. ((1994) Plant Physiol. 106:929-939) also have shown that introns are useful for regulating gene expression on a tissue specific basis. Plant cells transformed with such modified expression constructs or vectors, then, may exhibit overexpression or constitutive expression of a polynucleotide of the invention.

Expression constructs may additionally contain 5′ leader sequences. Such leader sequences can act to enhance translation. Translation leaders are known in the art and include: picornavirus leaders, for example, EMCV leader (Encephalomyocarditis 5′ noncoding region) (Elroy-Stein et al. (1989) Proc. Natl. Acad. ScL USA 86:6126-6130); potyvirus leaders, for example, TEV leader (Tobacco Etch Virus) (Gallie et al. (1995) Gene 165(2):233-238), MDMV leader (Maize Dwarf Mosaic Virus) (Virology 154:9-20), and human immunoglobulin heavy-chain binding protein (BiP) (Macejak et al. (1991) Nature 353: 90-94); untranslated leader from the coat protein mRNA of alfalfa mosaic virus (AMV RNA 4) (Jobling et al. (1987) Nature 325: 622-625); tobacco mosaic virus leader (TMV) (Gallie et al. (1989) in Molecular Biology of RNA, ed. Cech (Liss, New York), pp. 237-256); and maize chlorotic mottle virus leader (MCMV) (Lommel et al. (1991) Virology 81:382-385). See also, Della-Cioppa et al. (1987) Plant Physiol. 84:965-968.

In preparing the expression constructs and expression vectors described herein, the various polynucleotides may be manipulated, so as to provide for the polynucleotides in the proper orientation and, as appropriate, in the proper reading frame. Toward this end, adapters or linkers may be employed to join the nucleic acid molecules or other manipulations may be involved to provide for convenient restriction sites, removal of superfluous polynucleotides, removal of restriction sites, or the like. For this purpose, in vitro mutagenesis, primer repair, restriction, annealing, resubstitutions, e.g., transitions and transversions, may be involved.

Expression vectors may include additional features. For example, gRNA promoters to regulate expression of the at least one gRNA, e.g. prOsU3-01, which is the Rice U3 promoter for pol Ill dependent transcription of non-coding. Vectors may similarly include additional features such as selectable markers, e.g. Phosphomannose Isomerase (PMI), and antibiotic resistance genes that can be used to aid recovery of stably transformed plants.

By “operably linked” or “operably associated” as used herein, it is meant that the indicated elements are functionally related to each other, and are also generally physically related. Thus, the term “operably linked” or “operably associated” as used herein, refers to polynucleotides on a single nucleic acid molecule that are functionally associated. Thus, a first polynucleotide sequence or nucleic acid molecule that is operably linked to a second polynucleotide sequence or nucleic acid molecule, means a situation when the first polynucleotide sequence or nucleic acid molecule is placed in a functional relationship with the second polynucleotide sequence or nucleic acid molecule. For instance, a promoter is operably associated with a polynucleotide sequence or nucleic acid molecule if the promoter effects the transcription or expression of said polynucleotide sequence or nucleic acid molecule. Those skilled in the art will appreciate that the control sequences (e.g., promoter) need not be contiguous with the polynucleotide sequence or nucleic acid molecule to which it is operably associated, as long as the control sequences function to direct the expression thereof. Thus, for example, intervening untranslated, yet transcribed, sequences can be present between a promoter and a polynucleotide sequence or nucleic acid molecule, and the promoter can still be considered “operably linked” to or “operatively associated” with the polynucleotide sequence or nucleic acid molecule.

### Herbicide Resistance

The plants, parts thereof according to the invention have an increased resistance to a compound which inhibits the biotin synthesis pathway when compared to control plants, not expressing the polypeptides of the invention. The present invention further relates to a method of controlling the growth of undesired vegetation in the vicinity of such a plant according to the present invention, the method comprising applying an effective amount of at least one compound which inhibits the biotin synthesis pathway.

Suitably the compound which inhibits the biotin synthesis pathway is a herbicide. By ‘herbicide’ it is meant a chemical compound which is toxic to plants, typically used to control undesired vegetation such as weeds. Suitably therefore the plants of the invention are resistant to a herbicide which inhibits the biotin synthesis pathway, and therefore such plants can be used in methods where these herbicides are applied.

Suitably the compound which inhibits the biotin synthesis pathway may inhibit any one or more of the enzymes in the biotin synthesis pathway. Suitably in the plant biotin synthesis pathway. Suitably therefore, the compound inhibits one or more of the following enzymes: Pimeloyl CoA synthetase, KAPA synthetase (BIO4), DAPA aminotransferase (BIO1), DTB synthetase (BIO3), bifunctional dethiobiotin synthetase (BIO3-BIO1), and biotin synthetase (BIO2). Suitably the compound may inhibit more than one of the enzymes in this pathway.

Optionally the compound may inhibit a biochemical pathway upstream or downstream of the biotin synthesis pathway. For example, the compound may inhibit the upstream fatty acid synthesis pathway or polyketide synthesis pathway.

In one embodiment the compound inhibits bifunctional dethiobiotin synthetase (BIO3-BIO1). In one embodiment, the herbicide inhibits bifunctional dethiobiotin synthetase (BIO3-BIO1). Suitably therefore the invention relates to increasing the resistance of plants to herbicides which inhibit bifunctional dethiobiotin synthetase (BIO3-BIO1).

A plant having increased resistance to a compound which inhibits the biotin synthesis pathway may be referred to as an “herbicide-tolerant” or “herbicide-resistant” plant. Such plants are tolerant or at least partially resistant to at least one compound which inhibits the biotin synthesis pathway at a level that would normally kill, or inhibit the growth of, a normal, control or wild-type plant lacking the BIO3-BIO1 or BioA enzymes, polynucleotides encoding said enzymes or expression vectors of the invention. Generally, the term “herbicide” is used herein to mean an active ingredient that kills, controls or otherwise adversely modifies the growth of plants.

For example, plants of the invention may have at least a 2-fold increase in resistance to a compound which inhibits the biotin synthesis pathway, such as the inhibiting herbicides described herein. For example, plants of the invention may have at least a 2-fold, 3-fold, 4-fold, 5-fold 6-fold, 7-fold, 8-fold, 9-fold, 10-fold increase in resistance. In one embodiment, the plants of the invention may have at least a 2-fold increase in resistance to any of compounds A to M described herein, compared to an unmodified plant. In one embodiment, the plants of the invention may have at least a 2-fold, 3-fold, 4-fold, 5-fold 6-fold, 7-fold, 8-fold, 9-fold, 10-fold increase in resistance to any of compounds A to M described herein, compared to an unmodified plant.

Resistance to compounds which inhibit the biotin synthesis pathway may be determined by any known methods for comparing the growth, damage or other properties of two plants after application of compound which inhibits the biotin synthesis pathway to a plant. For example, the resistance of a plant of the invention may be determined by comparing the percentage of damaged caused to the plant in comparison to a wild-type or control plant after application of an compound which inhibits the biotin synthesis pathway, such as the herbicides described herein.

Without being bound by theory, increased resistance of the plant may be provided by an increase in the activity of the BIO3-BIO1 and/or BioA enzymes of the invention in the plant, in comparison to wild-type plants. This increase in activity may be provided by modification of the enzymes and/or overexpression of the enzymes in the plant. In one embodiment, as described above, the BIO3-BIO1 enzyme in the plant is a modified enzyme. In one embodiment, the BioA enzyme is overexpressed in the plant, and may optionally comprise a modification. The BIO3-BIO1 and/or BioA enzymes may also be less susceptible to inhibiting herbicides. In the case of BIO3-BIO1 this may be due to modifications in the structure of the enzyme leading to reduced binding of inhibiting herbicides, for BioA this may be due to the bacterial origins of this enzyme which may have a different structure to that of the plant enzymes to which the herbicides are targeted.

Increased resistance to a compound which inhibits the biotin synthesis pathway refers in the context of the invention to a plant which has been modified to comprise a BIO3-BIO1 and/or BioA enzyme, where the plant has an improved or increased resistance to a compound which inhibits the biotin synthesis pathway, relative to an unmodified plant. Suitably the increased resistance may be due to an increased activity of the BIO3-BIO1 and/or BioA enzyme in the plant, compared to the activity of such enzymes in an unmodified plant, when in the presence of at least one compound that is known to interfere with BIO3-BIO1 enzyme activity in plants at a concentration or level that is to known to inhibit the activity of the wild-type BIO3-BIO1 protein. Improved resistance means that the plant comprising the BIO3-BIO1 and/or BioA enzyme has an increase in resistance to a compound which inhibits the biotin synthesis pathway when compared to a plant not expressing the BIO3-BIO1 or BioA enzyme of the invention. Partially resistant plants of the invention may still have some decrease in biotin synthesis when exposed to a compound which inhibits the biotin synthesis pathway, such as at most a 1%, 2%, 3%, 4%, 5%, 6%, 7%, 8%, 9%, 10%, 15%, 20%, 25%, 30%, 35%, 40%, 45%, 50%, decrease in enzymatic activity. Suitably therefore the BIO3-BIO1 and/or BioA enzymes used in the invention may be partially resistant, and may still have some decrease in enzymatic activity when exposed to a compound which inhibits the biotin synthesis pathway. In some embodiments, the plants modified to comprise the enzymes may have total or near total resistance to a compound which inhibits the biotin synthesis pathway. Suitably the plants may have a statistically significant increase in resistance to the compound that inhibits biotin synthesis, including for example, at least a 60%, 70%, 80%, 90%, 91%, 92%, 93%, 94%, 95%, 96%, 97%, 98%, 99% or 100% resistance to a compound which inhibits the biotin synthesis pathway. Suitably therefore the BIO3-BIO1 and/or BioA enzymes used in the invention may have no substantial decrease in enzymatic activity when exposed to a compound which inhibits the biotin synthesis pathway.

However, any decreases in activity is less than a decrease in activity relative to the activity of a wild-type BIO3-BIO1 or BioA protein, when in the presence of at least one compound that is known to interfere with the biotin synthesis pathway and at a concentration or level of the compound that is to known to inhibit the activity of the wild-type BIO3-BIO1 protein. A decrease in activity seen for a partially resistant BIO3-BIO1 or BioA enzyme may be a decrease in activity that does not have a negative effect on the growth, propagation or development of a plant comprising a partially resistant BIO3-BIO1 and/or BioA enzyme. Furthermore, the activity of such a partially resistant BIO3-BIO1 and/or BioA protein may be referred to herein as “herbicide-tolerant” or “herbicide-resistant” enzyme. Plants which are at least partially “resistant” to at least one compound that inhibits the biotin synthesis pathway the exhibit few, if any, necrotic, lytic, chlorotic or other lesions when subjected to the compound at concentrations and rates which are typically employed by the agricultural community to kill unwanted vegetation in the vicinity of the plant such as a field.

The BIO3-BIO1 or BioA enzymes described herein may be compared to a reference, unmodified, or wild-type enzyme, which otherwise may be termed a control enzyme. The term “wild-type” is used to refer to a nucleic acid molecule or protein that can be found in nature as distinct from being artificially produced or mutated by man. A reference or unmodified BIO3-BIO1 enzyme or BioA enzyme may be a BIO3-BIO1 enzyme or BioA enzyme derived from the same source species as the modified enzyme, that does not include any modifications of the invention as described herein. In some embodiments, the reference enzyme may be a wild type enzyme. However, the use of the term “unmodified” or “reference” is not intended to necessarily imply that a plant, plant tissue, plant cell, or other host cell lacks any recombinant DNA in its genome, and/or does not possess herbicide resistant characteristics that are different from those disclosed herein.

Reference, or unmodified BIO3-BIO1 or BioA enzymes as referred to herein may well include other mutations or modifications that do not affect resistance to compounds which inhibit the biotin synthesis pathway. For example, a reference enzyme may include mutations or modifications to improve or alter expression, translation or targeting of the control enzyme to specific tissues, organs or cells.

A wild-type, reference unmodified BIO3-BIO1 enzyme may be an enzyme encoded by SEQ ID NO: 1. A wild-type, reference unmodified BioA enzyme may be a BioA enzyme encoded by SEQ ID NO: 159.

Many herbicides which inhibit the biotin synthesis pathway are known in the art and include a structurally diverse range of chemistry. Accordingly, the skilled person will appreciate that a broad range of biotin pathway inhibiting herbicides have utility in the present invention, especially BIO3-BIO1 inhibiting herbicides.

In one embodiment, the compound, suitably the herbicide, which inhibits the biotin synthesis pathway is selected from the group consisting of:
(i) a herbicidal cinnoline compound referred to in WO2021/233786, which is expressly incorporated herein by reference, for example ethyl 5-acetyl-4-oxo-1-(2,2,3,3-tetrafluoro-1,4-benzodioxin-6-yl)cinnoline-3-carboxylate (Compound A);
(ii) a herbicidal compound referred to in WO2022/064490, for example [5-carboxy-1-(1-hydroxyethyl)pentyl]ammonium chloride (Compound B);
(iii) a herbicidal pyridone compound disclosed in WO2022/117445, which is expressly incorporated herein by reference, for example 2-(3,4-dichlorophenyl)-5-(2,4-difluorophenyl)-1-ethyl-6-methyl-4-oxo-pyridine-3-carboxylic acid (Compound C) or 5-[2-chloro-5-(trifluoromethoxy)phenyl]-2-(3,4-dichlorophenyl)-1-ethyl-6-methyl-4-oxo-pyridine-3-carboxylic acid (Compound D);
(iv) a herbicidal compound referred to in WO2022/157780, which is expressly incorporated herein by reference, for example [(1R)-2-(6-carboxypyridin-1-ium-2-yl)oxy-1-methyl-ethyl]ammonium dichloride (Compound E);
(v) a herbicidal pyrrolidine-2-one referred to in WO2022/200208, which is expressly incorporated herein by reference, for example a compound selected from the group consisting of 2-[1-[(2,3-difluorophenyl)methyl]-5-oxo-pyrrolidin-2-yl]-N-(2-methyl-1,2,4-triazol-3-yl)acetamide (Compound F), 2-[1-[(2,3-difluorophenyl)methyl]-5-oxo-pyrrolidin-2-yl]acetic acid (Compound G), 2-[5-oxo-1-[(2,3,5-trifluorophenyl)methyl]pyrrolidin-2-yl]acetic acid (Compound H), 2-(4-fluorophenoxy)ethyl 2-[1-[(2,3-difluorophenyl)methyl]-5-oxo-pyrrolidin-2-yl]acetate (Compound I), 2-methoxyethyl 2-[1-[(2,3-difluorophenyl)methyl]-5-oxo-pyrrolidin-2-yl]acetate (Compound J), 2-[5-oxo-1-[(2,3,4-trifluorophenyl)methyl]pyrrolidin-2-yl]acetic acid (Compound K);
(vi) a N-benzyl azole herbicidal compound referred to in WO2023/110664, which is expressly incorporated herein by reference, for example 2-(3,4-dichlorophenyl)-1-ethyl-4-oxo-6-[[3-(trifluoromethyl)pyrazol-1-yl]methyl]pyridine-3-carboxylic acid (Compound L) and
(vii) a herbicidal quinolone compound referred to in WO2023/156323, which is expressly incorporated herein by reference, for example 2-(3,4-dichlorophenyl)-1-ethyl-6-fluoro-4-oxo-quinoline-3-carboxylic acid (Compound M).

Note that compounds A to M are tested in the examples below.

Other biotin pathway inhibiting herbicides which have utility in the present invention include, for example, cinnoline compounds disclosed in WO2021/233787 which is expressly incorporated herein by reference, pyridone compounds disclosed in WO2022/117446 which is expressly incorporated herein by reference, cinnoline compounds disclosed in WO2023/088921 which is expressly incorporated herein by reference, pyridone compounds disclosed in WO2023208710 which is expressly incorporated herein by reference, pyridone compounds disclosed in WO2023232673 which is expressly incorporated herein by reference, pyridone compounds disclosed in WO2023232674 which is expressly incorporated herein by reference and pyridone compounds disclosed WO2023232676 which is expressly incorporated herein by reference.

The compound which inhibits the biotin synthesis pathways may be any combination of one or more such inhibitory compounds, suitably one or more of the compounds listed above. For example, 1, 2, 3, 4, 5 or more of the inhibitory compounds as described herein.

The plants of the invention, that include a BIO3-BIO1 and/BioA enzyme of the invention, may be used in methods of controlling undesired vegetation in the vicinity of the plant. The methods may include applying an effective amount of at least one compound, such as those listed above, which inhibits the biotin synthesis pathway to the undesired vegetation and the plant.

In addition, plants of the invention, that include a BIO3-BIO1 and/or BioA enzyme of the invention may be used in methods of enhancing plant growth by controlling undesired vegetation in the vicinity of the plant. The methods may include applying an effective amount at least one compound, such as those listed above, which inhibits the biotin synthesis pathway to the undesired vegetation and the plant.

Suitably certain modified BIO3-BIO1 enzymes may perform better when used with certain compounds which inhibit the biotin synthesis pathway. Suitably certain modified BIO3-BIO1 enzymes may provide better resistance to certain compounds which inhibit the biotin synthesis pathway compared to other compounds which inhibit the biotin synthesis pathway. Suitably therefore certain combinations of BIO3-BIO1 modifications and compounds may be optimal for use in the methods of the invention.

In one embodiment of such methods, the plant of the invention may comprise a BIO3-BIO1 enzyme having a mutation at position F348 of SEQ ID NO:1, or at a corresponding position thereto, and the at least one compound is selected from compounds L, M, A, C and E. Suitably in such an embodiment, the BIO3-BIO1 enzyme may comprise one of the following mutations F348C, F348D, F348N, F348S, F348T, F348V, F348A, F348E, F3481, F348K, F348Q in SEQ ID NO:1, or corresponding mutations thereto. Suitably in such an embodiment, the plant comprises an increase in resistance to at least one of compounds L, M, A, C and E compared to an unmodified plant.

In one embodiment of such methods, the plant of the invention may comprise a BIO3-BIO1 enzyme having a mutation at position C388 of SEQ ID NO:1, or at a corresponding position thereto, and the at least one compound is selected from any of compounds A to M. Suitably in such an embodiment, the BIO3-BIO1 enzyme may comprise the mutation C388D in SEQ ID NO:1, or a corresponding mutation thereto. Suitably in such an embodiment, the plant comprises an increase in resistance to at least one of compounds A to M compared to an unmodified plant.

In one embodiment of such methods, the plant of the invention may comprise a BIO3-BIO1 enzyme having a mutation at position W392 of SEQ ID NO:1, or at a corresponding position thereto, and the at least one compound is selected from compounds L, M, C and B. Suitably in such an embodiment, the BIO3-BIO1 enzyme may comprise the mutation W392S in SEQ ID NO:1, or a corresponding mutation thereto. Suitably in such an embodiment, the plant comprises an increase in resistance to at least one of compounds L, M, C and B compared to an unmodified plant.

In one embodiment of such methods, the plant of the invention may comprise a BIO3-BIO1 enzyme having a mutation at position Y511 of SEQ ID NO:1, or at a corresponding position thereto, and the at least one compound is selected from compounds A and L. Suitably in such an embodiment, the BIO3-BIO1 enzyme may comprise the mutation Y511E in SEQ ID NO:1, or a corresponding mutation thereto. Suitably in such an embodiment, the plant comprises an increase in resistance to at least one of compounds A and L compared to an unmodified plant.

In one embodiment of such methods, the plant of the invention may comprise a BIO3-BIO1 enzyme having a mutation at position S509 of SEQ ID NO:1, or at a corresponding position thereto, and the at least one compound is selected from compounds L, A, D, B and E. Suitably in such an embodiment, the BIO3-BIO1 enzyme may comprise the mutation S509V in SEQ ID NO:1, or a corresponding mutation thereto. Suitably in such an embodiment, the plant comprises an increase in resistance to at least one of compounds L, A, D, B and E compared to an unmodified plant.

The control of undesired vegetation is understood as meaning the killing of undesired vegetation and/or otherwise retarding or inhibiting the normal growth of the undesired vegetation. Undesired vegetation, in the broadest sense, refers to all those plants which grow in locations where they are undesired.

Undesired vegetation may include, for example, dicotyledonous and monocotyledonous weeds. Dicotyledonous weeds include, but are not limited to, weeds of the genera: Sinapis, Lepidium, Galium, Slellaria, Matricaria, Anthemis, Galinsoga, Chenopodium, Urtica, Senecio, Amaranthus, Portulaca, Xanthium, Convolvulus, Ipomoea, Polygonum, Sesbania, Ambrosia, Cirsium, Carduus, Sonchus, Solanum, Rorippa, Rotala, Lindernia, Lamium, Veronica, Abutilon, Emex, Datura, Viola, Galeopsis, Papaver, Centaurea, Trifolium, Ranunculus, and Taraxacum. Monocotyledonous weeds include, but are not limited to, weeds of the genera: Echinochloa, Setaria, Panicum, Digitaria, Phleum, Poa, Festuca, Eleusine, Brachiaria, Lolium, Bromus, Avena, Cyperus, Sorghum, Agropyron, Cynodon, Monochoria, Fimbristyslis, Sagittaria, Eleocharis, Scirpus, Paspalum, Ischaemum, Sphenoclea, Dactyloctenium, Agrostis, Alopecurus, and Apera.

In addition, undesired vegetation can include, for example, crop plants that are growing in an undesired location. For example, a volunteer maize plant that is in a field that predominantly comprises soybean plants can be considered a weed, if the maize plant is undesired in the field of soybean plants.

An “effective amount” or “effective concentration” refers to an amount and concentration, respectively, of a compound that inhibits the biotin synthesis pathway, that is sufficient to kill or inhibit the growth of a similar, wild-type, plant, plant tissue, plant cell, microspore, or host cell, but that said amount does not kill or inhibit as severely the growth of the at least partially resistant plants, parts thereof, plant tissues, plant cells, and seeds of the invention. Typically, the effective amount is an amount that is routinely used in agricultural production systems to kill unwanted vegetation of interest. Such an amount is known to those of ordinary skill in the art, or can be easily determined using methods known in the art. Furthermore, it is recognized that the effective amount in an agricultural production system might be substantially different than an effective amount for a plant culture system such as, for example, the microspore culture system.

An effective amount may be at least 10 grams of active compound per hectare (g ai/ha). For example a compound that inhibits the biotin synthesis pathway may be applied at a concentration of at least 10, 15, 20, 25, 30, 35, 40, 45, 50, 55, 60, 65, 70, 75, 80, 85, 90, 95, 100,105,110,115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500 grams of active compound per hectare.

It should be understood that in the aforementioned methods the compound may be applied to the vicinity of the plant pre-emergence of the crop and/or post-emergence of the crop—a so-called “over-the-top” application.

“Preemergent” refers to compound which is applied to the vicinity of an at least partially resistant plant of the invention (e.g., a field or area of cultivation) before the plant emerges visibly from the soil and/or before germination of a seed. “Postemergent” refers to an compound which is applied to the vicinity of an at least partially resistant plant of the invention after a plant emerges visibly from the soil. In some instances, the terms “preemergent” and “postemergent” are used with reference to a weed or undesired vegetation in the vicinity of an at least partially resistant plant of the invention, and in some instances these terms are used with reference to a crop plant in the vicinity of an at least partially resistant plant of the invention. When used with reference to a weed or undesired vegetation, these terms may apply to only a particular type of weed or species of weed or undesired vegetation that is present or believed to be present in the area of interest. While any compound which inhibits the biotin synthesis pathway may be applied in a preemergent and/or postemergent treatment, some such compounds are known to be more effective in controlling a weed or weeds or undesired plants when applied either preemergence or postemergence. The compound may be applied “preplant incorporation” which involves the incorporation of the compound into the soil prior to planting.

The rates of application of a compound which inhibits the biotin synthesis pathway may vary within wide limits and depend on the nature of the soil, the method of application (pre-emergence; post-emergence; application to the seed furrow; no tillage application etc.), the plant, the undesired vegetation to be controlled, the prevailing climatic conditions, and other factors governed by the method of application, the time of application and the target plant. The compound which inhibits the biotin synthesis pathway may be applied at a rate of at least 10 L/ha. For example at least 10, 15, 20, 25, 30, 35, 40, 45, 50, 55, 60, 65, 70, 75, 80, 85, 90, 95, 100,105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000 L/ha. In some examples, the compound may be applied at a rate of 200 L/ha.

The application is generally made by spraying the compound, typically by tractor mounted sprayer for large areas, but other methods such as dusting (for powders), drip or drench can also be used.

According to the present invention, “enhancing plant growth of a plant” means an improvement in plant vigour, an improvement in plant quality, improved tolerance to stress factors, and/or improved input use efficiency.

The invention describe herein also relates to a kit comprising a container and instructions for use, the container comprising a compound which inhibits the biotin synthesis pathway, and the instructions comprising a direction to apply the compound to a plant modified to comprise a BIO3-BIO1 enzyme and/or BioA enzyme that provides the plant or part thereof with increased resistance to said compound.

Suitably the compound may be any one of those as defined above. Suitably the direction to apply the compound may comprise direction to apply the compound in an effective amount as defined above. Suitably the direction to apply the compound may comprise direction to apply the compound at a particular rate as described above. Suitably the direction to apply the compound may comprise direction to apply the compound in a particular method such as by spraying as defined above. Suitably the direction to apply the compound may comprise direction to apply the compound at a particular time such as pre-emergence of the crop and/or post-emergence of the crop, or during a particular season or month.

Suitably the instructions may further comprise direction to prepare the compound such that it can be applied in the desired effective amount and by the desired method. Suitably such directions may include direction to dilute the compound, suitably in a solvent such as water, suitably to an effective concentration.

### Methods of Screening

Further provided are various methods of screening for plants having an increased resistance to a compound which inhibits the biotin synthesis pathway, for modified BIO3-BIO1 enzyme and/or a BioA enzyme which comprises an increased resistance to a compound which inhibits the biotin synthesis pathway, and also for compounds which inhibits the biotin synthesis pathway.

In one aspect of the invention there is provided a method of selecting a plant having an increased resistance to a compound which inhibits the biotin synthesis pathway relative to an unmodified plant, comprising: (a) providing a plant; (b) optionally mutagenizing the plant; (c) exposing the plant to an effective amount of a compound which inhibits the biotin synthesis pathway; and (d) selecting the plant if the plant displays resistance to the compound.

In one embodiment, the plant may be a plant according to the first aspect of the invention, and may comprise any of the features defined in relation to such a plant recited herein.

Suitably step (b) may comprise mutagenizing the plant by any known means such as EMS or other chemical treatment, or by X-ray or another radiation treatment. Suitably to induce modifications, suitably one or more mutations, in the BIO3-BIO1 gene. Suitably the one or more mutations be any of those describe hereinabove.

Suitably after step (b), the plant comprises one or more modifications to the endogenous BIO3-BIO1 gene, suitably one or more mutations to the endogenous BIO3-BIO1 gene. Suitably the modified BIO3-BIO1 gene may encode a modified BIO3-BIO1 enzyme, suitably as described herein. Suitably therefore, after step (b) the plant may comprise a modified BIO3-BIO1 enzyme as described herein, suitably having one or more mutations as described herein.

Suitably step (c) comprises exposing the plant to an effective amount of a compound as described hereinabove. Suitably to determine if the one or more modifications to the endogenous BIO3-BIO1 gene, and the resulting modified BIO3-BIO1 enzyme, created in step (b) confer resistance to the compound.

Suitably selecting the plant if the plant displays resistance to the compound may comprise selecting those plants that do not display any signs of biotin deficiency. Suitably such signs may include stunted growth, wilting, necrosis, discolouration, and the like. In one embodiment, the sign is reduced growth compared to an unmodified plant. Suitably therefore selecting the plant if the plant displays resistance to the compound may comprise selecting those plants that retain a wild type phenotype, suitably which retain normal growth.

Further provided is a method of identifying a modified BIO3-BIO1 enzyme and/or a BioA enzyme which comprises an increased resistance to a compound which inhibits the biotin synthesis pathway, comprising: (a) generating a library of modified BIO3-BIO1 and/or BioA encoding polynucleotides; (b) screening a population of the resulting modified BIO3-BIO1 and/or BioA encoding polynucleotides by expressing each of said polynucleotides in a bacteria, a plant or a plant part and exposing the bacteria, plant or part thereof to an effective amount of a compound which inhibits the biotin synthesis pathway; (c) selecting the modified BIO3-BIO1 and/or BioA encoding polynucleotides which provide the bacteria, plant or plant part thereof with increased resistance to said compound compared to an reference bacteria, plant or plant part thereof containing an unmodified BIO3-BIO1 and/or BioA encoding polynucleotide.

Suitably generating a library of modified BIO3-BIO1 and/or BioA encoding polynucleotides may be carried out by any known technique to generate a library of genes comprising different modifications, suitably mutations throughout the gene sequence. Suitably these methods may be random or directed. Such methods may include a step of exposing the BIO3-BIO1 and/or BioA encoding polynucleotides, such as SEQ ID NOs:156 to 158, or 202 to 203, to a mutagen such as a chemical or radiation, or carrying out error prone PCR on the BIO3-BIO1 and/or BioA encoding polynucleotides for example. Other molecular techniques may include performing DNA shuffling or staggered extension processes on BIO3-BIO1 and/or BioA encoding polynucleotides, such as SEQ ID NOs:156 to 158, or 202 to 203.

Suitably expressing the modified polynucleotides in a bacteria, a plant or a plant part, such that the modified BIO3-BIO1 and/or BioA enzymes are expressed, comprises transforming a bacteria, a plant or a plant part with each of the modified polynucleotides. Suitable means of transformation are described elsewhere herein, as are suitable constructs/vectors for expression of a BIO3-BIO1 and/or BioA encoding polynucleotide.

Suitably exposing the bacteria, plant or part thereof to an effective amount of a compound which inhibits the biotin synthesis pathway comprises using an effective amount as described hereinabove, of a suitable compound as described hereinabove.

Suitably selecting the modified BIO3-BIO1 and/or BioA encoding polynucleotides which provide the bacteria, plant or plant part thereof with increased resistance to said compound, comprises selecting those host bacteria, plants or plant parts thereof that display resistance to the compound, suitably selecting those that do not display any signs of biotin deficiency. Suitably such signs in plants may include stunted growth, wilting, necrosis, discolouration, and the like. Suitably such signs in bacteria may include stunted growth or death. In one embodiment, the sign is reduced growth compared to an unmodified bacteria or unmodified plant.

Also provided is a method of identifying a compound which inhibits the biotin synthesis pathway comprising: (a) generating a modified plant or part as described herein, (b) applying a test compound to the plant or part thereof of step (a) and to an unmodified reference plant; (c) selecting the test compounds which confer reduced growth to the unmodified reference plant as compared to the growth of the modified plant or part thereof.

In one embodiment, the modified plant or plant part is a plant having increased resistance to compounds which inhibit the biotin synthesis pathway, as defined according to the first aspect of the invention, and may comprise any of the features defined in relation to such a plant recited herein.

Suitably the test compound may be any compound which may have, or which is expected to have an inhibitory effect on the biotin synthesis pathway of the plant. Suitably the test compound may be related to, derived from, or synthesised from a compound as defined herein, which may be a known compound which inhibits the biotin synthesis pathway, such as a known herbicidal compound.

Suitably the test compound may be applied in a test amount, suitably the test amount may be the same as any known effective amount for such herbicidal compounds, suitable amounts are defined hereinabove.

In another aspect of the invention, there are provided one or more compounds identified by such a method, optionally for use as herbicides.

The invention may further be defined by one or more of the following non-limiting numbered paragraphs:
1. A plant, or part thereof, modified to comprise a BIO3-BIO1 enzyme and/or BioA enzyme that provides the plant or part thereof with increased resistance to a compound which inhibits the biotin synthesis pathway relative to an unmodified reference plant.
2. A plant, or part thereof, according to paragraph 1, wherein the plant or a part thereof is modified to comprise a polynucleotide encoding a BIO3-BIO1 and/or BioA enzyme, the expression of which provides the plant or part thereof with increased resistance to a compound which inhibits the biotin synthesis pathway relative to an unmodified plant.
3. A plant, or part thereof, according to paragraph 1 wherein the plant or part thereof is modified to increase expression of the BIO3-BIO1 enzyme and/or a BioA enzyme, preferably wherein the plant or part thereof is modified to overexpress the BIO3-BIO1 enzyme and/or a BioA enzyme.
4. A plant, or part thereof, according to paragraph 2 or 3, wherein the expression of the BIO3-BIO1 enzyme and/or a BioA enzyme is increased or overexpressed to a level which provides the plant or part thereof with increased resistance to a compound which inhibits the biotin synthesis pathway relative to an unmodified plant, preferably wherein the expression of the BIO3-BIO1 enzyme and/or a BioA enzyme is increased or overexpressed to a level of at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% greater than the expression of the BIO3-BIO1 enzyme and/or a BioA enzyme in an unmodified reference plant.
5. A plant, or part thereof, according to any preceding paragraph wherein the BIO3-BIO1 enzyme and/or the BioA enzyme is a wild type enzyme.
6. A plant, or part thereof, according to any preceding paragraph wherein the BIO3-BIO1 enzyme is derived from a plant, from an algae, from an oomycete, or a from a fungus, preferably wherein the BIO3-BIO1 enzyme is derived from a plant, more preferably the BIO3-BIO1 enzyme is derived from any of the following plant species: Setaria italica, Arabidopsis thaliana, Helianthus annuus, Quercus robur, Phoenix dactylifera, Physcomitrium patens, Taxus chinensis, Adiantum nelumboides, Zea mays, Ostreococcus tauri, Hordeum vulgare, Brassica napus, Gossypium hirsutum, Oryza sativa, Glycine max, and Triticum aestivum.
7. A plant, or part thereof, according to any preceding paragraph wherein the BioA enzyme is derived from a bacterium, protist, or archaeon, preferably the BioA enzyme is derived from any of the following bacterial, protist, or archaeon species: Escherichia coli, Cryptosporidium andersoni, Agrobacterium tumefaciens, Citrobacter portucalensis, Cedecea sp. nfix57 BioA, Xenorhabdus sp. xeno-1, Methanobrevibacter olleyae, Bacillus subtilis, Pantoea ananatis, Bacillus licheniformis, Streptomyces hygroscopicus, Streptomyces viridochromogenes, Stenotrophomonas maltophilia, Pseudomonas fluorescens, Bacillus thuringiensis, Candidatus Midichloria mitochondrii, Thiofilum flexile, Panacibacter microcysteis, Spirosoma linguale, Prochlorococcus marinus, Chitinophaga polysaccharea, Chitinophaga filiformis, Pedobacter hartonius, Fluviicola sp xm-24bin1, Flavobacterium foetidum, Chroococcidiopsis sp. CCMEE 29, Tenacibaculum adriaticum, Pseudobacteriovorax antillogorgiicola, Texcoconibacillus texcoconensis, Nitrobacter sp. 62-13, Wigglesworthia glossinidia, Methylomarinum vadi, Flocculibacter collagenilyticus, Leptolyngbya ectocarpi, Psychrosphaera aestuarii, Fragilariopsis cylindrus, Deferrisoma camini, Chlorobaculum tepidum, Chlamydia pneumoniae, Pedobacter psychrophilus, and Pseudopedobacter saltans.
8. A plant, or part thereof, according to any preceding paragraph wherein the BIO3-BIO1 enzyme and the BioA enzyme are heterologous to the plant or part thereof.
9. A plant, or part thereof, according to any preceding paragraph wherein the BIO3-BIO1 enzyme and/or the BioA enzyme comprise any one or more of the following motifs:

[Motif 13: SEQ ID NO: 220]
(A/C/G/S); (F/Y); H; G; (D/E); T;(F/I/L/M/V/W);
(A/D/E/G/K/M/Q);(A/C/G/P/T/V);(I/L/M/V);
(A/D/E/N/S); (A/C/I/L/M/T/V);
[Motif 14: SEQ ID NO: 221]
(F/Y); (F/L/Y); (A/C/N/S/V); D; (D/N/S); G; (A/S);
(A/C/E/I/S/T/V); (A/C/G/S); (C/I/M/T/V); (D/E);
(C/I/V); (A/G/S);(I/L/M/V); K; (A/I/M/V);
(A/C/S/V);
and
(SEQ ID NO: 270)
(H/Q); (C/I/M/S/T/V); (I/L/M/T/V); (F/L/Y);
(A/G/L/P); (D/E/G/M/N); (A/C/F/I/L/M/N/Y);
(A/G/I/L/S/T/V); (H/N/Y); (E/K/N/P/Q/S/V);
(A/G/K/Q/P/T)

10. A plant, or part thereof, according to any preceding paragraph wherein the BIO3-BIO1 enzyme comprises any one or more of the following motifs:
   W;(H/Y/W);P;F;(A/Q/S/T);Q;(H/Q/V);X;X;X [Motif 1 (SEQ ID NO:208)];
   (I/L/V);(D/E);(S/G);(R/A);X;(A/D/G/K);(E/D/N);X;(F/Y) [Motif 2 (SEQ ID NO:209)]; (F/I/L/V/Y);D;(A/G);(C/I/P/S);(A/G/S);S;W;W;(T/S/V);(I/Q) [Motif 3 (SEQ ID NO:210)];
   (F/Y);(G/D);(H/Q);(A/I/V);(M/I/L);(F/L/Y);(A/L/P);(E/G/H/L/N/S/T) [Motif 4 (SEQ ID NO:211)];
   (A/G/T);(F/L/T/Q/V);X;(G/N/D/E/R);(S/C/G);Y;H;G;D;T;(L/I/M);(G/S);(A/C/S/T/V);(M/L/T);(D/E/N);X;(A/E/K/Q/R/S/T);(A/E/I/Q/V/T);(E/G/I/K/P/S);(C/E/N/S/T);X;(F/T/Y);(M/N/S/T);X [Motif 5 (SEQ ID NO:212)];
   (A/E/K/Q/R/S/T);(E/H/I/Q/V/T);(D/E/H/P);(S/W);(F/H/Y);X;(G/P/Q/R/S);(E/K/Q/R/W) [Motif 6 (SEQ ID NO:213)];
   (F/S/W);(F/I/L/P);X;(A/C/F/P/V/Y);(P/S);X;(I/L/M/T/V) [Motif 7 (SEQ ID NO:214)];
   (A/G);(A/S);(A/G);G;M;X;(F/M/L);(A/C/I/V) [Motif 8 (SEQ ID NO:215)];
   L;L;H;(G/R);H;S;(F/Y);(N/S/T);(A/G/T);(H/N/Y) [Motif 9 (SEQ ID NO:216)];
   (A/E/G/H/K/P/Q/S/V);A/C/G/K/N/Q/R/S/T/V/Y);(H/I/L/V);(A/D/E/H/K/L/N/Q/R/S);(G/K/R/S/Y);(A/C/I/L/T/V);(F/1/M/T/V/W);(A/D/G/N/S/V;(I/L/M/Q/V) [Motif 10 (SEQ ID NO:217)];
   (A/I/L/V/Y);(A/I/L/N/Q/R/V);(A/D/E/I/K/L/M/N/T/Q/R/S);(A/D/E/F/H/K/M/N/Q/R/S/T/V/Y);(F/L);(A/H/K/L/M/R/S/T/Y);X;X;(F/G) [Motif 11 (SEQ ID NO:218)]; and
   (A/I/L/M/N/V);(F/H/L/Q/Y);(A/C/E/I/L/M/S/T);R;(A/1/P/S/V);L;G;(D/K/N;I/T/V);(F/I/L/M/V);Y [Motif 12 (SEQ ID NO:219)], wherein ‘X’ indicates any amino acid.
11. A plant, or part thereof, according to any preceding paragraph wherein the BIO3-BIO1 enzyme comprises or consists of a sequence having at least 30% identity to any of SEQ ID NOs 1 to 14, 271-276, or a functional fragment thereof.
12. A plant, or part thereof, according to any preceding paragraph wherein the BioA enzyme comprises any one or more of the following motifs:

(SEQ ID NO: 268)
(A/G/S); (F/Y); H; G; (D/E); T; (F/I/L/M/V/W);
(A/D/E/G/K/M/Q); (A/G/P/T); (I/L/M/V); (A/E/S);
(A/I/L/T/V);
(SEQ ID NO: 269)
D; E; (I/V); (A/F/L/M); (T/V); G; (F/L/W); G;
(K/R); (C/S/T); G; (A/E/K/L/Q/P/R/S/T);
(F/L/M/N/R/W); F; (A/G/S)

13. A plant, or part thereof, according to any preceding paragraph wherein the BioA enzyme comprises or consists of a sequence having at least 30% identity to any of SEQ ID NOs 159-159, or a functional fragment thereof.
14. A plant, or part thereof, according to any of paragraph 1-4, 6-13, wherein the plant or part thereof is modified to comprise a BIO3-BIO1 enzyme and/or BioA enzyme having one or more mutations, preferably wherein the or each mutation is an amino acid substitution.
15. A plant, or part thereof, according to paragraph 14 wherein the or each mutation provides the plant or part thereof with increased resistance to a compound which inhibits the biotin synthesis pathway relative to an unmodified plant.
16. A plant, or part thereof, according to paragraph 14 or 15, wherein the BIO3-BIO1 enzyme comprises one or more amino acid substitutions at any position in any of the following motifs:
   W;(H/Y/W);P;F;(A/Q/S/T);Q;(H/Q/V);X;X;X [Motif 1 (SEQ ID NO:208)];
   (I/L/V);(D/E);(S/G);(R/A);X;(A/D/G/K);(E/D/N);X;(F/Y) [Motif 2 (SEQ ID NO:209)];
   (F/I/L/V/Y);D;(A/G);(C/1/P/S);(A/G/S);S;W;W;(T/S/V);(I/Q) [Motif 3 (SEQ ID NO:210)]; (F/Y);(G/D);(H/Q);(A/1/V);(M/I/L);(F/L/Y);(A/L/P);(E/G/H/L/N/S/T) [Motif 4 (SEQ ID NO:211)];
   (A/G/T);(F/L/T/Q/V);X;(G/N/D/E/R);(S/C/G);Y;H;G;D;T;(L/I/M);(G/S); (A/C/S/T/V);(M/L/T); (D/E/N);X;(A/E/K/Q/R/S/T); (A/E/I/Q/V/T);(E/G/I/K/P/S); (C/E/N/S/T);X;(F/T/Y); (M/N/S/T);X [Motif 5 (SEQ ID NO:212)];
   (A/E/K/Q/R/S/T);(E/H/I/Q/V/T);(D/E/H/P);(S/W);(F/H/Y);X;(G/P/Q/R/S);(E/K/Q/R/W) [Motif 6 (SEQ ID NO:213)];
   (F/S/W);(F/I/L/P);X;(A/C/F/P/V/Y);(P/S);X;(I/L/M/T/V) [Motif 7 (SEQ ID NO:214)];
   (A/G);(A/S);(A/G);G;M;X;(F/M/L);(A/C/I/V) [Motif 8 (SEQ ID NO:215)];
   L;L;H;(G/R);H;S;(F/Y);(N/S/T);(A/G/T);(H/N/Y) [Motif 9 (SEQ ID NO:216)];
   (A/E/G/H/K/P/Q/S/V);A/C/G/K/N/Q/R/S/T/V/Y);(H/I/L/V);(A/D/E/H/K/L/N/Q/R/S);(G/K/R/S/Y);(A/C/I/L/T/V);(F/I/M/T/V/W);(A/D/G/N/S/V;(I/L/M/Q/V) [Motif 10 (SEQ ID NO:217)];
   (A/I/L/V/Y);(A/I/L/N/Q/R/V);(A/D/E/I/K/L/M/N/T/Q/R/S);(A/D/E/F/H/K/M/N/Q/R/S/T/V/Y);(F/L);(A/H/K/L/M/R/S/T/Y);X;X;(F/G) [Motif 11 (SEQ ID NO:218)]; and
   (A/I/L/M/N/V);(F/H/L/Q/Y);(A/C/E/I/L/M/S/T);R;(A/I/P/S/V);L;G;(D/K/N;I/T/V);(F/I/L/M/V);Y
   [Motif 12 (SEQ ID NO:219)], wherein ‘X’ indicates any amino acid.
17. A plant, or part thereof, according to paragraph 16 wherein the BIO3-BIO1 enzyme comprises an amino acid substitution at one or more of the following positions:
   The third, fourth, sixth and/or tenth residue in motif 1, preferably wherein the BIO3-BIO1 enzyme comprises the modified motif 1:

(SEQ ID NO: 226)
W; (H/Y/W); (A/E); (A/C/D/E/I/K/M/N/Q/S/T/V);
(A/Q/S/T); (H/S); (H/Q/V); X; X;(A/E/L/N/T);

The ninth residue in motif 2, preferably wherein the BIO3-BIO1 enzyme comprises the modified motif 2:

(SEQ ID NO: 227)
(I/L/V); (D/E); (S/G); (R/A); X; (A/D/G/K);
(E/D/N); X; L;

The fourth, fifth, sixth, seventh, eighth, and/or ninth residue in motif 3, preferably wherein the BIO3-BIO1 enzyme comprises the modified motif 3:
(F/I/L/V/Y);D;(A/G);(D/M/T); F;C;(FIL/M);(A/C/DIGIM/S);V;(I/Q) (SEQ ID NO:234);
The fifth, sixth, and/or seventh, residue in motif 4, preferably wherein the BIO3-BIO1 enzyme comprises the modified motif 4:

(SEQ ID NO: 238)
(F/Y); (G/D); (H/Q); (A/I/V); I; I; (A/E/G/L/W);
(E/G/H/L/N/S/T);

The fifth, seventeenth, eighteenth, nineteenth, twentieth, twenty-first, twenty-second, twenty-third, and/or twenty-fourth residue in motif 5, preferably wherein the BIO3-BIO1 enzyme comprises the modified motif

(SEQ ID NO: 249)
(A/G/T); (F/L/T/Q/V); X; (G/N/D/E/R); A; Y; S; G;
D; T; (L/I/M); (G/S); (A/C/S/T/V); (M/L/T);
(D/E/N); X; A; (K/S); (DELETION/L/T);
(A/C/D/E/F/G/H/I/K/L/M/N/Q/R/S/T/V/W/Y);
(A/C/E/L/Q/V); (C/D/E/F/H/I/K/M/P/Q/R/V/W);
(C/D/G/I/N/Q/R/V/W); (DELETION/A/L/P)

The first, second, and/or fifth residue in motif 6, preferably wherein the BIO3-BIO1 enzyme comprises the modified motif 6:

(SEQ ID NO: 253)
C; (D/F/H/I/M/T/W/Y); (D/E/H/P); (S/W); (N/W); X;
(G/P/Q/R/S); (E/K/Q/R/W);

The fourth residue in motif 7, preferably wherein the BIO3-BIO1 enzyme comprises the modified motif 7:

(SEQ ID NO: 254)
(F/S/W); (F/I/L/P); X; A; (P/S); X; (I/L/M/T/V);

The first, second third, and/or fifth residue in motif 8, preferably wherein the BIO3-BIO1 enzyme comprises the modified motif 8:

(SEQ ID NO: 259)
(A/E/I); (C/F/H/I/K/M/N/R/T/V/W/Y); H; G; L; X;
(F/M/L); (A/C/I/V);

The fourth and/or eighth reside in motif 9, preferably wherein the BIO3-BIO1 enzyme comprises the modified motif 9:

(SEQ ID NO: 262)
L; L; H; (A/C/S); H; S;(F/Y); (H/P); (A/G/T);
(H/N/Y);

The fifth residue in motif 10, preferably wherein the BIO3-BIO1 enzyme comprises the modified motif 10:

(SEQ ID NO: 263)
(A/E/G/H/K/P/Q/S/V); (A/C/G/K/N/Q/R/S/T/V/Y);
(H/I/L/V); (A/D/E/H/K/L/N/Q/R/S); (S/K);
(A/C/I/L/T/V); (F/I/M/T/V/W); (A/D/G/N/S/V;
(I/L/M/Q/V);

The second and/or sixth residue in motif 11, preferably wherein the BIO3-BIO1 enzyme comprises the modified motif 11:
(A/I/L/V/Y);S;(A/D/E/I/K/L/M/N/T/Q/R/S);(A/D/E/F/H/K/M/N/Q/R/S/T/V/Y);(F/L);C;X;X;(F/G) (SEQ ID NO:266); and
The fourth residue in motif 12, preferably wherein the BIO3-BIO1 enzyme comprises the modified motif 12:
(A/I/L/M/N/V);(F/H/L/Q/Y);(A/C/E/I/L/M/S/T);Q;(A/I/P/S/V);L;G;(D/K/N;I/T/V);(F/I/L/M/V);Y (SEQ ID NO:267), wherein ‘X’ indicates any amino acid.
18. A plant, or part thereof, according to any of paragraph 14 to17, wherein the BIO3-BIO1 enzyme comprises an amino acid sequence having at least 30% identity to SEQ ID NO:1, and comprises an amino acid substitution at one or more of the following positions: P347, F348, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, and R797 of SEQ ID NO:1, or at corresponding positions thereto.
19. A plant, or part thereof, according to any preceding paragraph wherein the BIO3-BIO1 enzyme and/or the BioA enzyme is modified to comprise a heterologous targeting peptide, preferably a heterologous mitochondrial targeting peptide.
20. A modified BIO3-BIO1 enzyme having one or more mutations which provide increased resistance to a compound which inhibits the biotin synthesis pathway relative to an unmodified reference BIO3-BIO1 enzyme.
21. A modified BIO3-BIO1 enzyme according to paragraph 20, wherein the modified BIO3-BIO1 enzyme comprises one or more amino acid substitutions at any position in any of the following motifs:
   W;(H/Y/W);P;F;(A/Q/S/T);Q;(H/Q/V);X;X;X [Motif 1 (SEQ ID NO:208)];
   (I/L/V);(D/E);(S/G);(R/A);X;(A/D/G/K);(E/D/N);X;(F/Y) [Motif 2 (SEQ ID NO:209)];
   (F/I/L/V/Y);D;(A/G);(C/I/P/S);(A/G/S);S;W;W;(T/S/V);(I/Q) [Motif 3 (SEQ ID NO:210)];
   (F/Y);(G/D);(H/Q);(A/I/V);(M/I/L);(F/L/Y);(A/L/P);(E/G/H/L/N/S/T) [Motif 4 (SEQ ID NO:211)];
   (A/G/T);(F/L/T/Q/V);X;(G/N/D/E/R);(S/C/G);Y;H;G;D;T;(L/I/M); (G/S);(A/C/S/T/V);(M/L/T);(D/E/N);X;(A/E/K/Q/R/S/T);(A/E/I/Q/V/T);(E/G/I/K/P/S);(C/E/N/S/T);X;(F/T/Y);(M/N/S/T);X [Motif 5 (SEQ ID NO:212)];
   (A/E/K/Q/R/S/T);(E/H/I/Q/V/T);(D/E/H/P);(S/W);(F/H/Y);X;(G/P/Q/R/S);(E/K/Q/R/W) [Motif 6 (SEQ ID NO:213)];
   (F/S/W);(F/I/L/P);X;(A/C/F/P/V/Y);(P/S);X;(I/L/M/T/V) [Motif 7 (SEQ ID NO:214)];
   (A/G);(A/S);(A/G);G;M;X;(F/M/L);(A/C/I/V) [Motif 8 (SEQ ID NO:215)];
   L;L;H;(G/R);H;S;(F/Y);(N/S/T);(A/G/T);(H/N/Y) [Motif 9 (SEQ ID NO:216)];
   (A/E/G/H/K/P/Q/S/V);A/C/G/K/N/Q/R/S/T/V/Y);(H/I/L/V);(A/D/E/H/K/L/N/Q/R/S);(G/K/R/S/Y);(A/C/I/L/T/V);(F/I/M/T/V/W);(A/D/G/N/S/V;(I/L/M/Q/V) [Motif 10 (SEQ ID NO:217)];
   (A/I/L/V/Y);(A/I/L/N/Q/R/V);(A/D/E/I/K/L/M/N/T/Q/R/S);(A/D/E/F/H/K/M/N/Q/R/S/T/V/Y);(F/L);(A/H/K/L/M/R/S/T/Y);X;X;(F/G) [Motif 11 (SEQ ID NO:218)]; and
   (A/I/L/M/N/V);(F/H/L/Q/Y);(A/C/E/I/L/M/S/T);R;(A/I/P/S/V);L;G;(D/K/N;I/T/V);(F/I/L/M/V);Y [Motif 12 (SEQ ID NO:219)], wherein ‘X’ indicates any amino acid.
22. A modified BIO3-BIO1 enzyme according to paragraph 21, wherein the modified BIO3-BIO1 enzyme comprises an amino acid substitution at one or more of the following positions:
   The third, fourth, sixth and/or tenth residue in motif 1, preferably wherein the BIO3-BIO1 enzyme comprises the modified motif 1:

(SEQ ID NO: 226)
W; (H/Y/W); (A/E); (A/C/D/E/I/K/M/N/Q/S/T/V);
(A/Q/S/T); (H/S); (H/Q/V); X; X;(A/E/L/N/T);

The ninth residue in motif 2, preferably wherein the BIO3-BIO1 enzyme comprises the modified motif 2:

(SEQ ID NO: 227)
(I/L/V); (D/E); (S/G); (R/A); X; (A/D/G/K);
(E/D/N); X; L;

The fourth, fifth, sixth, seventh, eighth, and/or ninth residue in motif 3, preferably wherein the BIO3-BIO1 enzyme comprises the modified motif 3:

(SEQ ID NO: 234)
(F/I/L/V/Y); D; (A/G); (D/M/T); F; C; (F/L/M);
(A/C/D/G/M/S); V; (I/Q);

The fifth, sixth, and/or seventh, residue in motif 4, preferably wherein the BIO3-BIO1 enzyme comprises the modified motif 4:

(SEQ ID NO: 238)
(F/Y); (G/D); (H/Q); (A/I/V); I; I; (A/E/G/L/W);
(E/G/H/L/N/S/T);

The fifth, seventeenth, eighteenth, nineteenth, twentieth, twenty-first, twenty-second, twenty-third, and/or twenty-fourth residue in motif 5, preferably wherein the BIO3-BIO1 enzyme comprises the modified motif

(SEQ ID NO: 249)
(A/G/T); (F/L/T/Q/V); X; (G/N/D/E/R); A; Y; S; G;
D; T; (L/I/M); (G/S); (A/C/S/T/V); (M/L/T);
(D/E/N); X; A; (K/S); (DELETION/L/T);
(A/C/D/E/F/G/H/I/K/L/M/N/Q/R/S/T/V/W/Y);
(A/C/E/L/Q/V); (C/D/E/F/H/I/K/M/P/Q/R/V/W);
(C/D/G/I/N/Q/R/V/W); (DELETION/A/L/P);

The first, second, and/or fifth residue in motif 6, preferably wherein the BIO3-BIO1 enzyme comprises the modified motif 6:

(SEQ ID NO: 253)
C; (D/F/H/I/M/T/W/Y); (D/E/H/P); (S/W); (N/W); X;
(G/P/Q/R/S); (E/K/Q/R/W);

The fourth residue in motif 7, preferably wherein the BIO3-BIO1 enzyme comprises the modified motif 7:

(SEQ ID NO: 254)
(F/S/W); (F/I/L/P); X; A; (P/S); X; (I/L/M/T/V);

The first, second third, and/or fifth residue in motif 8, preferably wherein the BIO3-BIO1 enzyme comprises the modified motif 8:

(SEQ ID NO: 259)
(A/E/I); (C/F/H/I/K/M/N/R/T/V/W/Y); H; G; L; X;
(F/M/L); (A/C/I/V);

The fourth and/or eighth reside in motif 9, preferably wherein the BIO3-BIO1 enzyme comprises the modified motif 9:

(SEQ ID NO: 262)
L; L; H; (A/C/S); H; S; (F/Y); (H/P); (A/G/T);
(H/N/Y);

The fifth residue in motif 10, preferably wherein the BIO3-BIO1 enzyme comprises the modified motif 10:

(SEQ ID NO: 263)
(A/E/G/H/K/P/Q/S/V); (A/C/G/K/N/Q/R/S/T/V/Y);
(H/I/L/V); (A/D/E/H/K/L/N/Q/R/S); (S/K);
(A/C/I/L/T/V); (F/I/M/T/V/W); (A/D/G/N/S/V;
(I/L/M/Q/V);

The second and/or sixth residue in motif 11, preferably wherein the BIO3-BIO1 enzyme comprises the modified motif 11:
(A/I/L/V/Y);S;(A/D/E/1/K/L/M/N/T/Q/R/S);(A/D/E/F/H/K/M/N/Q/R/S/T/V/Y);(F/L);C;X;X;(F/G) (SEQ ID NO:266); and
The fourth residue in motif 12, preferably wherein the BIO3-BIO1 enzyme comprises the modified motif 12:
(A/I/L/M/N/V); (F/H/L/Q/Y);(A/C/E/I/L/M/S/T); Q;(A/1/P/S/V);L;G; (D/K/N;I/T/V);(F/I/L/M/V);Y (SEQ ID NO:267), wherein ‘X’ indicates any amino acid.
23. A modified BIO3-BIO1 enzyme according to any of paragraph 20 to 22, wherein the modified BIO3-BIO1 enzyme comprises an amino acid sequence having at least 30% identity to SEQ ID NO:1, and comprises an amino acid substitution at one or more of the following positions: P347, F348, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, and R797 of SEQ ID NO:1, or at corresponding positions thereto.
24. An isolated polynucleotide encoding a modified BIO3-BIO1 enzyme according to any of paragraph 20 to 23.
25. An expression construct comprising a polynucleotide encoding a modified BIO3-BIO1 enzyme of any of paragraph 20 to 23 and/or a polynucleotide of paragraph 24, operably linked to one or more expression elements.
26. A vector comprising the expression construct of paragraph 25.
27. A method of producing a plant or part thereof according to any of paragraph 1-19, the method comprising: modifying the plant or part thereof to comprise a BIO3-BIO1 enzyme and/or BioA enzyme that provides increased resistance to a compound which inhibits the biotin synthesis pathway relative to an unmodified reference plant.
28. A method according to paragraph 27, wherein modifying the plant or part thereof comprises transforming the plant or part thereof with a polynucleotide encoding a BIO3-BIO1 enzyme and/or BioA enzyme the expression of which provides increased resistance to a compound which inhibits the biotin synthesis pathway relative to an unmodified reference plant.
29. A method of controlling undesired vegetation in the vicinity of a plant or part thereof according to any of paragraph 1-19, or at the locus for growth of a plant according to any of paragraph 1-19, the method comprising applying an effective amount of at least one compound which inhibits the biotin synthesis pathway to the undesired vegetation and the plant, or the locus, and optionally planting a seed at the locus wherein the seed is capable of producing a plant according to any of paragraph 1-19.
30. Use of a compound which inhibits the biotin synthesis pathway in combination with a plant or part thereof according to any of paragraph 1 to 19.
31. A plant, or part thereof according to any of paragraph 1-19, a method of producing a plant or part thereof according to paragraph 27 or 28, a method of controlling undesired vegetation according to paragraph 29, and a use according to paragraph 30, wherein the compound which inhibits the biotin synthesis pathway inhibits one or more of the enzymes in the biotin synthesis pathway or related pathways, preferably wherein the compound inhibits one or more of pimeloyl CoA synthetase, KAPA synthase, DAPA synthase, DTB synthetase, and biotin synthase.
32. A plant, or part thereof according to any of paragraph 1-19, a method of producing a plant or part thereof according to paragraph 27 or 28, a method of controlling undesired vegetation according to paragraph 29, and a use according to paragraph 30, wherein the compound is a herbicide, preferably wherein the compound is a herbicide selected from: a herbicidal cinnoline compound such as ethyl 5-acetyl-4-oxo-1-(2,2,3,3-tetrafluoro-1,4-benzodioxin-6-yl)cinnoline-3-carboxylate (Compound A); a herbicidal compound such as [5-carboxy-1-(1-hydroxyethyl)pentyl]ammonium;chloride (compound B); a herbicidal pyridone compound such as 2-(3,4-dichlorophenyl)-5-(2,4-difluorophenyl)-1-ethyl-6-methyl-4-oxo-pyridine-3-carboxylic acid (Compound C) or 5-[2-chloro-5-(trifluoromethoxy)phenyl]-2-(3,4-dichlorophenyl)-1-ethyl-6-methyl-4-oxo-pyridine-3-carboxylic acid (Compound D); a herbicidal compound such as [(1R)-2-(6-carboxypyridin-1-ium-2-yl)oxy-1-methyl-ethyl]ammonium;dichloride (Compound E); a herbicidal pyrrolidine-2-one such as 2-[1-[(2,3-difluorophenyl)methyl]-5-oxo-pyrrolidin-2-yl]-N-(2-methyl-1,2,4-triazol-3-yl)acetamide (Compound F), 2-[1-[(2,3-difluorophenyl)methyl]-5-oxo-pyrrolidin-2-yl]acetic acid (Compound G), 2-[5-oxo-1-[(2,3,5-trifluorophenyl)methyl]pyrrolidin-2-yl]acetic acid (Compound H), 2-(4-fluorophenoxy)ethyl 2-[1-[(2,3-difluorophenyl)methyl]-5-oxo-pyrrolidin-2-yl]acetate (Compound I), 2-methoxyethyl 2-[1-[(2,3-difluorophenyl)methyl]-5-oxo-pyrrolidin-2-yl]acetate (Compound J), 2-[5-oxo-1-[(2,3,4-trifluorophenyl)methyl]pyrrolidin-2-yl]acetic acid (Compound K); a N-benzyl azole herbicidal compound such as 2-(3,4-dichlorophenyl)-1-ethyl-4-oxo-6-[[3-(trifluoromethyl)pyrazol-1-yl]methyl]pyridine-3-carboxylic acid (Compound L); and a herbicidal quinolone compound such as 2-(3,4-dichlorophenyl)-1-ethyl-6-fluoro-4-oxo-quinoline-3-carboxylic acid (Compound M).
33. A plant, or part thereof according to any of paragraph 1-19, a method of producing a plant or part thereof according to paragraph 27 or 28, a method of controlling undesired vegetation according to paragraph 29, and a use according to paragraph 30, wherein increased resistance to the compound comprises an increase of at least 2-fold, 3-fold, 4-fold, 5-fold 6-fold, 7-fold, 8-fold, 9-fold, 10-fold in resistance to the compound compared to an unmodified reference plant or part thereof.

The invention may further be defined by one or more of the following non-limiting numbered paragraphs:
1. A plant, or part thereof, modified to comprise a BIO3-BIO1 enzyme and/or BioA enzyme that provides the plant or part thereof with increased resistance to a compound which inhibits the biotin synthesis pathway relative to an unmodified reference plant.
2. A plant, or part thereof, according to paragraph 1, wherein the plant or a part thereof is modified to comprise a polynucleotide encoding a BIO3-BIO1 and/or BioA enzyme, the expression of which provides the plant or part thereof with increased resistance to a compound which inhibits the biotin synthesis pathway relative to an unmodified plant.
3. A plant, or part thereof, according to paragraph 1 wherein the plant or part thereof is modified to increase expression of the BIO3-BIO1 enzyme and/or a BioA enzyme, preferably wherein the plant or part thereof is modified to overexpress the BIO3-BIO1 enzyme and/or a BioA enzyme.
4. A plant, or part thereof, according to paragraph 2 or 3, wherein the expression of the BIO3-BIO1 enzyme and/or a BioA enzyme is increased or overexpressed to a level which provides the plant or part thereof with increased resistance to a compound which inhibits the biotin synthesis pathway relative to an unmodified plant, preferably wherein the expression of the BIO3-BIO1 enzyme and/or a BioA enzyme is increased or overexpressed to a level of at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% greater than the expression of the BIO3-BIO1 enzyme and/or a BioA enzyme in an unmodified reference plant.
5. A plant, or part thereof, according to any preceding paragraph wherein the BIO3-BIO1 enzyme and/or the BioA enzyme is a wild type enzyme, and/or wherein the BIO3-BIO1 enzyme and/or the BioA enzyme is heterologous to the plant or part thereof.
6. A plant, or part thereof, according to any preceding paragraph wherein the BIO3-BIO1 enzyme and/or the BioA enzyme comprise any one or more of the following motifs:

[Motif 13: SEQ ID NO: 220]
(A/C/G/S); (F/Y); H;G; (D/E); T; (F/I/L/M/V/W);
(A/D/E/G/K/M/Q); (A/C/G/P/T/V); (I/L/M/V);
(A/D/E/N/S);(A/C/I/L/M/T/V);
[Motif 14: SEQ ID NO: 221]
(F/Y); (F/L/Y); (A/C/N/S/V); D; (D/N/S); G; (A/S);
(A/C/E/I/S/T/V); (A/C/G/S); (C/I/M/T/V); (D/E);
(C/I/V); (A/G/S); (I/L/M/V); K; (A/I/M/V);
(A/C/S/V);
and
(SEQ ID NO: 270)
(H/Q); (C/I/M/S/T/V); (I/L/M/T/V); (F/L/Y);
(A/G/L/P); (D/E/G/M/N); (A/C/F/I/L/M/N/Y);
(A/G/I/L/S/T/V); (H/N/Y); (E/K/N/P/Q/S/V);
(A/G/K/Q/P/T) (Motif 17)

7. A plant, or part thereof, according to any preceding paragraph wherein the BIO3-BIO1 enzyme comprises any one or more of the following motifs:
   W;(H/Y/W);P;F;(A/Q/S/T);Q;(H/Q/V);X;X;X [Motif 1 (SEQ ID NO:208)];
   (I/L/V);(D/E);(S/G);(R/A);X;(A/D/G/K);(E/D/N);X;(F/Y) [Motif 2 (SEQ ID NO:209)];
   (F/I/L/V/Y);D;(A/G);(C/I/P/S);(A/G/S);S;W;W;(T/S/V);(I/Q) [Motif 3 (SEQ ID NO:210)];
   (F/Y);(G/D);(H/Q);(A/I/V);(M/I/L);(F/L/Y);(A/L/P);(E/G/H/L/N/S/T) [Motif 4 (SEQ ID NO:211)];
   (A/G/T);(F/L/T/Q/V);X;(G/N/D/E/R);(S/C/G);Y;H;G;D;T;(L/I/M);(G/S);(A/C/S/T/V);(M/L/T);(D/E/N);X;(A/E/K/Q/R/S/T);(A/E/I/Q/V/T);(E/G/I/K/P/S);(C/E/N/S/T);X;(F/T/Y);(M/N/S/T);X [Motif 5 (SEQ ID NO:212)];
   (A/E/K/Q/R/S/T);(E/H/I/Q/V/T);(D/E/H/P);(S/W);(F/H/Y);X;(G/P/Q/R/S);(E/K/Q/R/W) [Motif 6 (SEQ ID NO:213)];
   (F/S/W);(F/I/L/P);X;(A/C/F/P/V/Y);(P/S);X;(I/L/M/T/V) [Motif 7 (SEQ ID NO:214)];
   (A/G);(A/S);(A/G);G;M;X;(F/M/L);(A/C/I/V) [Motif 8 (SEQ ID NO:215)];
   L;L;H;(G/R);H;S;(F/Y);(N/S/T);(A/G/T);(H/N/Y) [Motif 9 (SEQ ID NO:216)];
   (A/E/G/H/K/P/Q/S/V);A/C/G/K/N/Q/R/S/T/V/Y);(H/I/L/V);(A/D/E/H/K/L/N/Q/R/S);(G/K/R/S/Y);(A/C/I/L/T/V);(F/I/M/T/V/W);(A/D/G/N/S/V;(I/L/M/Q/V) [Motif 10 (SEQ ID NO:217)];
   (A/I/L/V/Y);(A/I/L/N/Q/R/V);(A/D/E/I/K/L/M/N/T/Q/R/S);(A/D/E/F/H/K/M/N/Q/R/S/T/V/Y);(F/L);(A/H/K/L/M/R/S/T/Y);X;X;(F/G) [Motif 11 (SEQ ID NO:218)]; and
   (A/I/L/M/N/V);(F/H/L/Q/Y);(A/C/E/I/L/M/S/T);R;(A/I/P/S/V);L;G;(D/K/N;I/T/V);(F/I/L/M/V);Y [Motif 12 (SEQ ID NO:219)], wherein ‘X’ indicates any amino acid.
8. A plant, or part thereof, according to any preceding paragraph wherein the BIO3-BIO1 enzyme comprises or consists of a sequence having at least 30% identity to any of SEQ ID NOs 1 to 14, 271-276, or a functional fragment thereof.
9. A plant, or part thereof, according to any preceding paragraph wherein the BioA enzyme comprises any one or more of the following motifs:

(SEQ ID NO: 268)
(A/G/S); (F/Y); H; G; (D/E); T; (F/I/L/M/V/W);
(A/D/E/G/K/M/Q); (A/G/P/T); (I/L/M/V); (A/E/S);
(A/I/L/T/V);
and
(SEQ ID NO: 269)
D; E; (I/V); (A/F/L/M); (T/V); G; (F/L/W); G;
(K/R); (C/S/T); G; (A/E/K/L/Q/P/R/S/T);
(F/L/M/N/R/W); F; (A/G/S)

10. A plant, or part thereof, according to any preceding paragraph wherein the BioA enzyme comprises or consists of a sequence having at least 30% identity to any of SEQ ID NOs 159-199, or a functional fragment thereof.
11. A plant, or part thereof, according to any preceding paragraph, wherein the plant or part thereof is modified to comprise a BIO3-BIO1 enzyme and/or BioA enzyme having one or more mutations, preferably wherein the or each mutation is an amino acid substitution.
12. A plant, or part thereof, according to paragraph 11 wherein the or each mutation provides the plant or part thereof with increased resistance to a compound which inhibits the biotin synthesis pathway relative to an unmodified plant.
13. A plant, or part thereof, according to paragraphs 11 or 12 wherein the BIO3-BIO1 enzyme comprises an amino acid substitution at one or more of the following positions:
   The third, fourth, sixth and/or tenth residue in motif 1, preferably wherein the BIO3-BIO1 enzyme comprises the modified motif 1:

(SEQ ID NO: 226)
W; (H/Y/W); (A/E); (A/C/D/E/I/K/M/N/Q/S/T/V);
(A/Q/S/T); (H/S); (H/Q/V); X; X;(A/E/L/N/T);

The ninth residue in motif 2, preferably wherein the BIO3-BIO1 enzyme comprises the modified motif 2:

(SEQ ID NO: 227)
(I/L/V); (D/E); (S/G); (R/A); X; (A/D/G/K);
(E/D/N); X; L;

The fourth, fifth, sixth, seventh, eighth, and/or ninth residue in motif 3, preferably wherein the BIO3-BIO1 enzyme comprises the modified motif 3:

(SEQ ID NO: 234)
(F/I/L/V/Y); D; (A/G); (D/M/T); F; C; (F/L/M);
(A/C/D/G/M/S); V; (I/Q);

The fifth, sixth, and/or seventh, residue in motif 4, preferably wherein the BIO3-BIO1 enzyme comprises the modified motif 4:

(SEQ ID NO: 238)
(F/Y); (G/D); (H/Q); (A/I/V); I; I; (A/E/G/L/W);
(E/G/H/L/N/S/T);

The fifth, seventeenth, eighteenth, nineteenth, twentieth, twenty-first, twenty-second, twenty-third, and/or twenty-fourth residue in motif 5, preferably wherein the BIO3-BIO1 enzyme comprises the modified motif

(SEQ ID NO: 249)
(A/G/T); (F/L/T/Q/V); X; (G/N/D/E/R); A; Y; S
; G; D; T; (L/I/M); (G/S); (A/C/S/T/V); (M/L/
T); (D/E/N); X; A; (K/S); (DELETION/L/T); (A/
C/D/E/F/G/H/I/K/L/M/N/Q/R/S/T/V/W/Y); (A/C/E/
L/Q/V); (C/D/E/F/H/I/K/M/P/Q/R/V/W); (C/D/G/I
/N/Q/R/V/W); (DELETION/A/L/P);

The first, second, and/or fifth residue in motif 6, preferably wherein the BIO3-BIO1 enzyme comprises the modified motif 6:
C;(D/F/H/I/M/T/W/Y);(D/E/H/P);(S/W);(N/W);X;(G/P/Q/R/S);(E/K/Q/R/W) (SEQ ID NO:253);
The fourth residue in motif 7, preferably wherein the BIO3-BIO1 enzyme comprises the modified motif 7:

(SEQ ID NO: 254)
(F/S/W); (F/I/L/P); X; A; (P/S); X; (I/L/M/T/
V);

The first, second third, and/or fifth residue in motif 8, preferably wherein the BIO3-BIO1 enzyme comprises the modified motif 8:

(SEQ ID NO: 259)
(A/E/I); (C/F/H/I/K/M/N/R/T/V/W/Y); H; G; L;
X; (F/M/L); (A/C/I/V);

The fourth and/or eighth reside in motif 9, preferably wherein the BIO3-BIO1 enzyme comprises the modified motif 9:

(SEQ ID NO: 262)
L; L; H; (A/C/S); H; S; (F/Y); (H/P); (A/G/T);
(H/N/Y);

The fifth residue in motif 10, preferably wherein the BIO3-BIO1 enzyme comprises the modified motif 10:

(SEQ ID NO: 263)
(A/E/G/H/K/P/Q/S/V); (A/C/G/K/N/Q/R/S/T/V/Y);
(H/I/L/V); (A/D/E/H/K/L/N/Q/R/S); (S/K); (A/
C/I/L/T/V); (F/I/M/T/V/W); (A/D/G/N/S/V; (I/L
/M/Q/V);

The second and/or sixth residue in motif 11, preferably wherein the BIO3-BIO1 enzyme comprises the modified motif 11:
(A/I/L/V/Y);S;(A/D/E/I/K/L/M/N/T/Q/R/S);(A/D/E/F/H/K/M/N/Q/R/S/T/V/Y);(F/L);C;X;X; (F/G) (SEQ ID NO:266); and
The fourth residue in motif 12, preferably wherein the BIO3-BIO1 enzyme comprises the modified motif 12:
(A/I/L/M/N/V);(F/H/L/Q/Y);(A/C/E/I/L/M/S/T);Q;(A/I/P/S/V);L;G;(D/K/N;I/T/V);(F/I/L/M/V);Y (SEQ ID NO:267), wherein ‘X’ indicates any amino acid.
14. A plant, or part thereof, according to any of paragraphs 11 to 13, wherein the BIO3-BIO1 enzyme comprises an amino acid sequence having at least 30% identity to SEQ ID NO:1, and comprises an amino acid substitution at one or more of the following positions: P347, F348, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, and R797 of SEQ ID NO:1, or at corresponding positions thereto.
15. A plant, or part thereof, according to any preceding paragraph wherein the BIO3-BIO1 enzyme and/or the BioA enzyme is modified to comprise a heterologous targeting peptide, preferably a heterologous mitochondrial targeting peptide.
16. A modified BIO3-BIO1 enzyme having one or more mutations which provide increased resistance to a compound which inhibits the biotin synthesis pathway relative to an unmodified reference BIO3-BIO1 enzyme.
17. A modified BIO3-BIO1 enzyme according to paragraph 16, wherein the modified BIO3-BIO1 enzyme comprises an amino acid substitution at one or more of the following positions:
   The third, fourth, sixth and/or tenth residue in motif 1, preferably wherein the BIO3-BIO1 enzyme comprises the modified motif 1:

(SEQ ID NO: 226)
W; (H/Y/W); (A/E); (A/C/D/E/I/K/M/N/Q/S/T/V);
(A/Q/S/T); (H/S); (H/Q/V); X; X; (A/E/L/N/T);

The ninth residue in motif 2, preferably wherein the BIO3-BIO1 enzyme comprises the modified motif 2:

(SEQ ID NO: 227)
(I/L/V); (D/E); (S/G); (R/A); X; (A/D/G/K);
(E/D/N); X; L;

The fourth, fifth, sixth, seventh, eighth, and/or ninth residue in motif 3, preferably wherein the BIO3-BIO1 enzyme comprises the modified motif 3:

(SEQ ID NO: 234)
(F/I/L/V/Y); D; (A/G); (D/M/T); F; C; (F/L/M);
(A/C/D/G/M/S); V; (I/Q);

The fifth, sixth, and/or seventh, residue in motif 4, preferably wherein the BIO3-BIO1 enzyme comprises the modified motif 4:

(SEQ ID NO: 238)
(F/Y); (G/D); (H/Q); (A/I/V); I; I; (A/E/G/L/
W); (E/G/H/L/N/S/T);

The fifth, seventeenth, eighteenth, nineteenth, twentieth, twenty-first, twenty-second, twenty-third, and/or twenty-fourth residue in motif 5, preferably wherein the BIO3-BIO1 enzyme comprises the modified motif

(SEQ ID NO: 249)
(A/G/T); (F/L/T/Q/V); X; (G/N/D/E/R); A; Y; S
; G; D; T; (L/I/M); (G/S); (A/C/S/T/V); (M/L/
T); (D/E/N); X; A; (K/S); (DELETION/L/T); (A/
C/D/E/F/G/H/I/K/L/M/N/Q/R/S/T/V/W/Y); (A/C/E/
L/Q/V); (C/D/E/F/H/I/K/M/P/Q/R/V/W); (C/D/G/I
/N/Q/R/V/W); (DELETION/A/L/P);

The first, second, and/or fifth residue in motif 6, preferably wherein the BIO3-BIO1 enzyme comprises the modified motif 6:

(SEQ ID NO: 253)
C; (D/F/H/I/M/T/W/Y); (D/E/H/P); (S/W); (N/W);
X; (G/P/Q/R/S); (E/K/Q/R/W);

The fourth residue in motif 7, preferably wherein the BIO3-BIO1 enzyme comprises the modified motif 7:

(SEQ ID NO: 254)
(F/S/W);(F/I/L/P); X; A; (P/S); X; (I/L/M/T/V);

The first, second third, and/or fifth residue in motif 8, preferably wherein the BIO3-BIO1 enzyme comprises the modified motif 8:

(SEQ ID NO: 229)
(A/E/I); (C/F/H/I/K/M/N/R/T/V/W/Y); H; G; L;
X; (F/M/L); (A/C/I/V);

The fourth and/or eighth reside in motif 9, preferably wherein the BIO3-BIO1 enzyme comprises the modified motif 9:

(SEQ ID NO: 262)
L; L; H; (A/C/S); H; S; (F/Y); (H/P); (A/G/T);
(H/N/Y);

The fifth residue in motif 10, preferably wherein the BIO3-BIO1 enzyme comprises the modified motif 10:

(SEQ ID NO: 263)
(A/E/G/H/K/P/Q/S/V); (A/C/G/K/N/Q/R/S/T/V/Y);
(H/I/L/V); (A/D/E/H/K/L/N/Q/R/S); (S/K);(A/C/
I/L/T/V); (F/I/M/T/V/W); (A/D/G/N/S/V; (I/L/M/
Q/V);

The second and/or sixth residue in motif 11, preferably wherein the BIO3-BIO1 enzyme comprises the modified motif 11:
(A/I/L/V/Y);S;(A/D/E/I/K/L/M/N/T/Q/R/S);(A/D/E/F/H/K/M/N/Q/R/S/T/V/Y);(F/L);C;X;X; (F/G) (SEQ ID NO:266); and
The fourth residue in motif 12, preferably wherein the BIO3-BIO1 enzyme comprises the modified motif 12:
(A/I/L/M/N/V); (F/H/L/Q/Y);(A/C/E/I/L/M/S/T); Q;(A/I/P/S/V);L;G; (D/K/N;I/T/V);(F/I/L/M/V);Y (SEQ ID NO:267), wherein ‘X’ indicates any amino acid.
18. A modified BIO3-BIO1 enzyme according to any of paragraphs 16 or 17, wherein the modified BIO3-BIO1 enzyme comprises an amino acid sequence having at least 30% identity to SEQ ID NO:1, and comprises an amino acid substitution at one or more of the following positions: P347, F348, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, and R797 of SEQ ID NO:1, or at corresponding positions thereto.
19. A method of producing a plant or part thereof according to any of paragraphs 1-18, the method comprising: modifying the plant or part thereof to comprise a BIO3-BIO1 enzyme and/or BioA enzyme that provides increased resistance to a compound which inhibits the biotin synthesis pathway relative to an unmodified reference plant.
20. A method according to paragraph 19, wherein modifying the plant or part thereof comprises transforming the plant or part thereof with a polynucleotide encoding a BIO3-BIO1 enzyme and/or BioA enzyme the expression of which provides increased resistance to a compound which inhibits the biotin synthesis pathway relative to an unmodified reference plant.
21. A method of controlling undesired vegetation in the vicinity of a plant or part thereof according to any of paragraphs 1-18, or at the locus for growth of a plant according to any of paragraphs 1-18, the method comprising applying an effective amount of at least one compound which inhibits the biotin synthesis pathway to the undesired vegetation and the plant, or the locus, and optionally planting a seed at the locus wherein the seed is capable of producing a plant according to any of paragraphs 1-18.
22. A plant, or part thereof according to any of paragraphs 1-18, a method of producing a plant or part thereof according to paragraphs 19 or 20, or a method of controlling undesired vegetation according to paragraph 21, wherein the compound is a herbicide, preferably wherein the compound is a herbicide selected from: a herbicidal cinnoline compound such as ethyl 5-acetyl-4-oxo-1-(2,2,3,3-tetrafluoro-1,4-benzodioxin-6-yl)cinnoline-3-carboxylate (Compound A); a herbicidal compound such as [5-carboxy-1-(1-hydroxyethyl)pentyl]ammonium;chloride (compound B); a herbicidal pyridone compound such as 2-(3,4-dichlorophenyl)-5-(2,4-difluorophenyl)-1-ethyl-6-methyl-4-oxo-pyridine-3-carboxylic acid (Compound C) or 5-[2-chloro-5-(trifluoromethoxy)phenyl]-2-(3,4-dichlorophenyl)-1-ethyl-6-methyl-4-oxo-pyridine-3-carboxylic acid (Compound D); a herbicidal compound such as [(1R)-2-(6-carboxypyridin-1-ium-2-yl)oxy-1-methyl-ethyl]ammonium;dichloride (Compound E); a herbicidal pyrrolidine-2-one such as 2-[1-[(2,3-difluorophenyl)methyl]-5-oxo-pyrrolidin-2-yl]-N-(2-methyl-1,2,4-triazol-3-yl)acetamide (Compound F), 2-[1-[(2,3-difluorophenyl)methyl]-5-oxo-pyrrolidin-2-yl]acetic acid (Compound G), 2-[5-oxo-1-[(2,3,5-trifluorophenyl)methyl]pyrrolidin-2-yl]acetic acid (Compound H), 2-(4-fluorophenoxy)ethyl 2-[1-[(2,3-difluorophenyl)methyl]-5-oxo-pyrrolidin-2-yl]acetate (Compound I), 2-methoxyethyl 2-[1-[(2,3-difluorophenyl)methyl]-5-oxo-pyrrolidin-2-yl]acetate (Compound J), 2-[5-oxo-1-[(2,3,4-trifluorophenyl)methyl]pyrrolidin-2-yl]acetic acid (Compound K); a N-benzyl azole herbicidal compound such as 2-(3,4-dichlorophenyl)-1-ethyl-4-oxo-6-[[3-(trifluoromethyl)pyrazol-1-yl]methyl]pyridine-3-carboxylic acid (Compound L); and a herbicidal quinolone compound such as 2-(3,4-dichlorophenyl)-1-ethyl-6-fluoro-4-oxo-quinoline-3-carboxylic acid (Compound M).

### BRIEF DESCRIPTION OF FIGURES

FIG. 1 shows: An alignment of plant, oomycete, fungal and algal BIO3-BIO1 (BioDA) amino acid sequences from Setaria italica (XP_034598866.1) (SEQ ID NO:7), Schizosaccharomyces japonicus (XP_002171944.1) (SEQ ID NO: 13), Arabidopsis thaliana (NP_200567.2) (SEQ ID NO:1), Helianthus annus (XP_022027934.1) (SEQ ID NO:10), Quercus robur (XP_050272527.1) (SEQ ID NO:11), Pheonix dactylifera (XP_017700051.1) (SEQ ID NO:8), Physcomitrium patens (XP_024400978.1) (SEQ ID NO:5), Taxus chinensis (KAH9305032.1) (SEQ ID NO:4), Adiantum nelumboides (MC05565847) (SEQ ID NO:6), Nannochloropsis gaditana (EWM27173.1) (SEQ ID NO:3), Ostreococcus tauri (OUS46258.1) (SEQ ID NO:9), Gibberella zeae (DAA33949.1) (SEQ ID NO:14), Thraustotheca clavata (OQR99718.1) (SEQ ID NO:12), Zea mays (XP_008662155.1) (SEQ ID NO:2), Hordeum vulgare (XP_044951020.1) (SEQ ID NO:271), Brassica napus (RID41192.1) (SEQ ID NO:272), Gossypium hirsutum (XP_040941738.1) (SEQ ID NO:273), Oryza sativa (XP_015650723.1) (SEQ ID NO:274), Glycine max (XP_006593545.1) (SEQ ID NO:275), Selaginella moellendorffii (SEQ ID NO: 319) and Triticum aestivum (XP_044412124.1) (SEQ ID NO:276), generated using Geneious as a global alignment with free end gaps having the following parameters: cost matrix Blosum62, gap open penalty 12, gap extension penalty 3, refinement iterations 2.

FIG. 2 shows: A distance matrix showing the percentage sequence identity between the BIO3-BIO1 amino acid sequences of the alignment of FIG. 1, with A to T defined as follows:

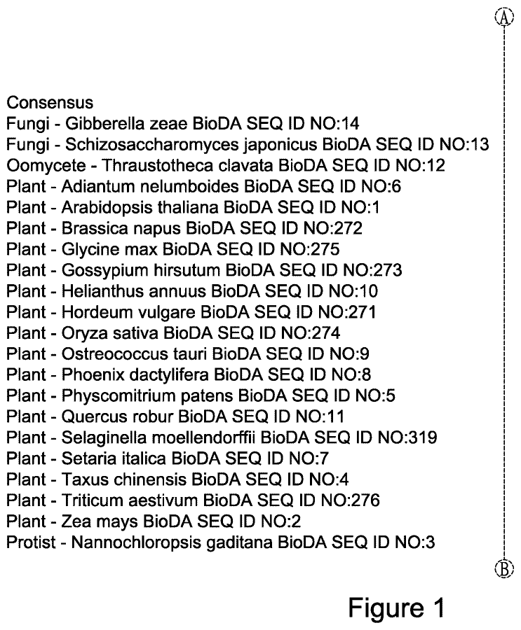

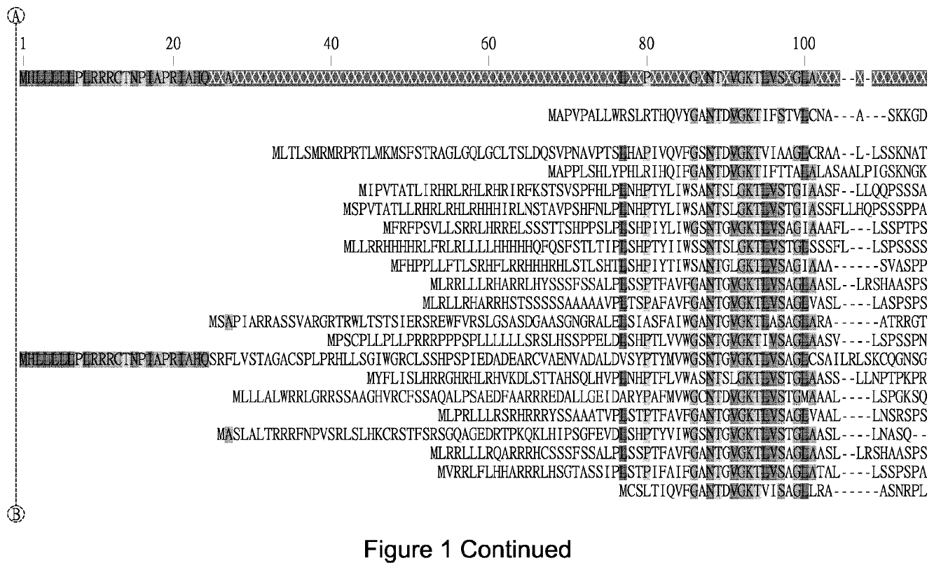

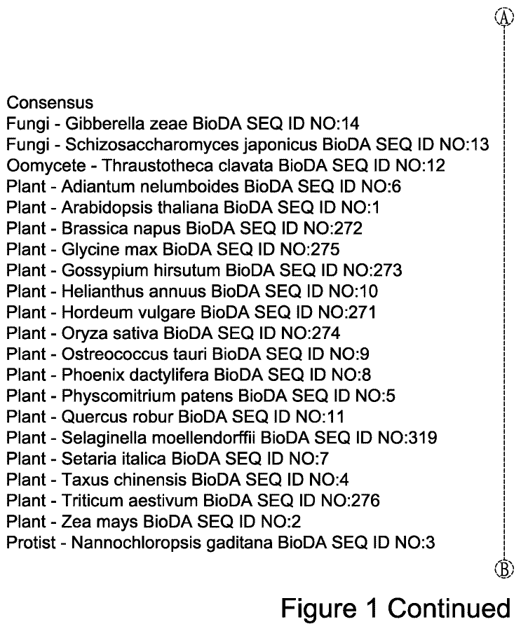

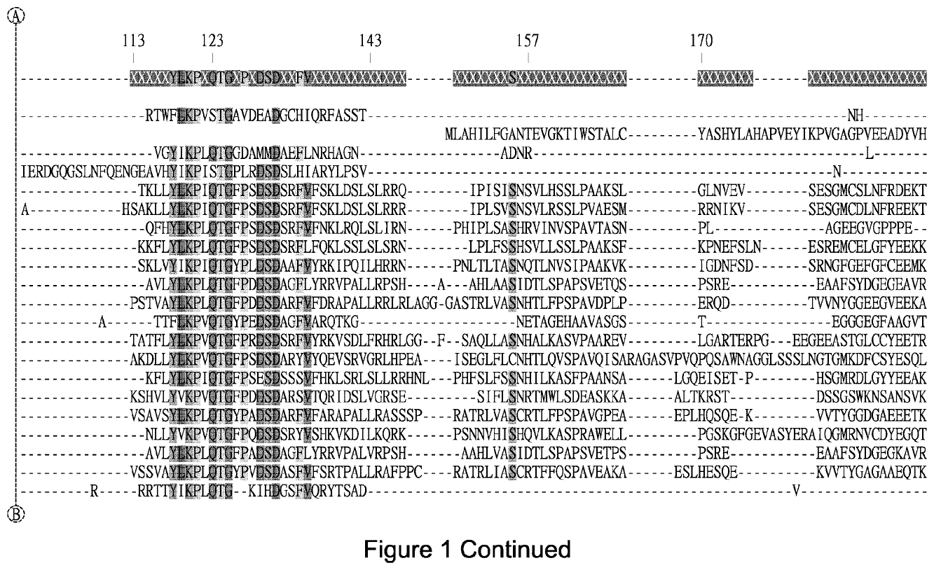

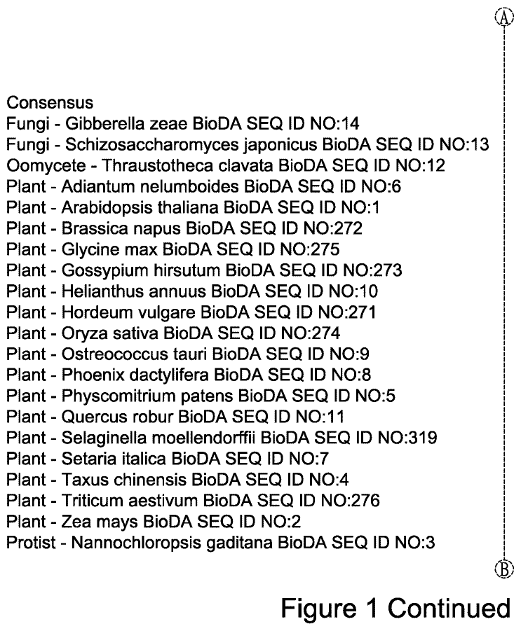

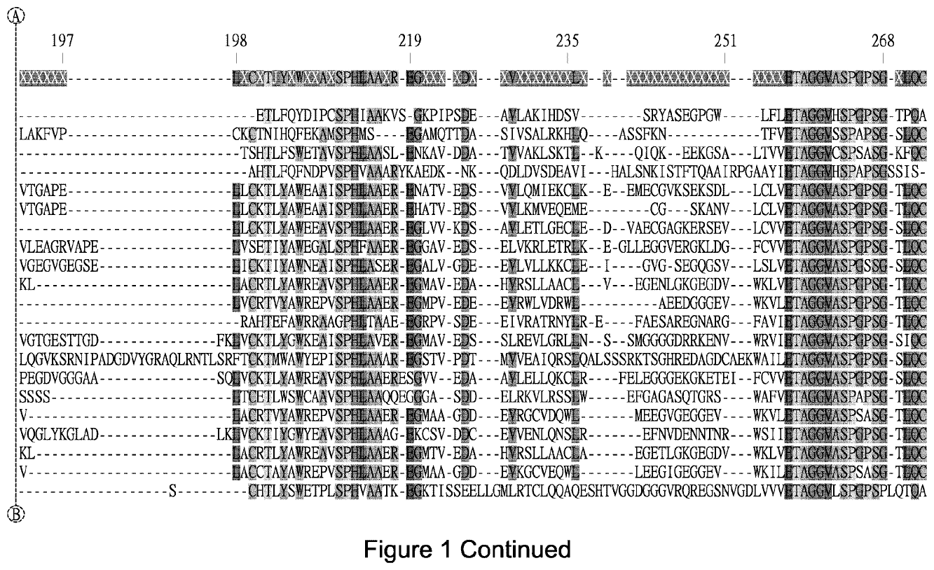

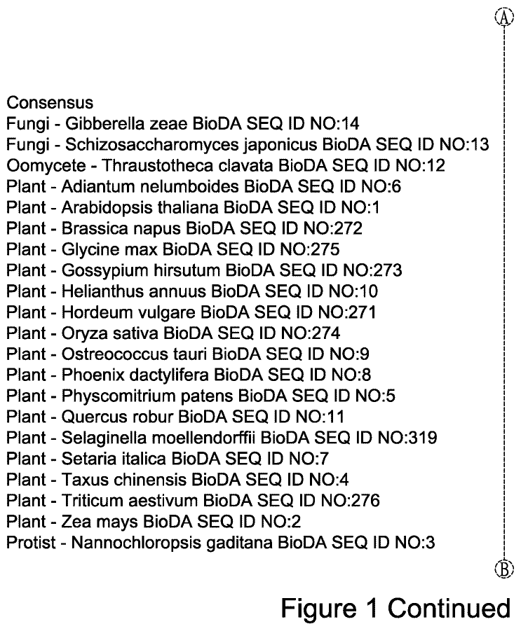

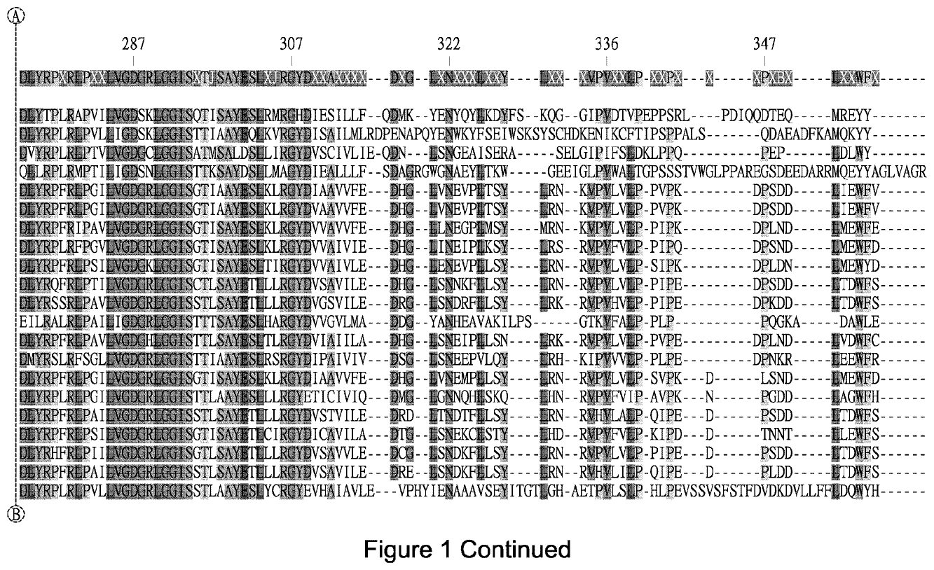

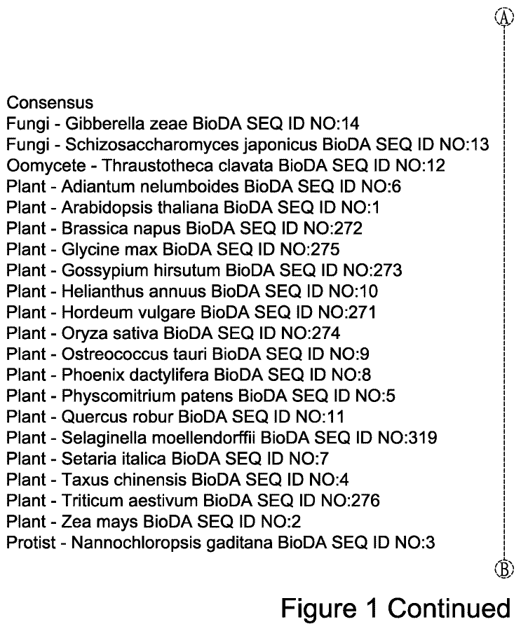

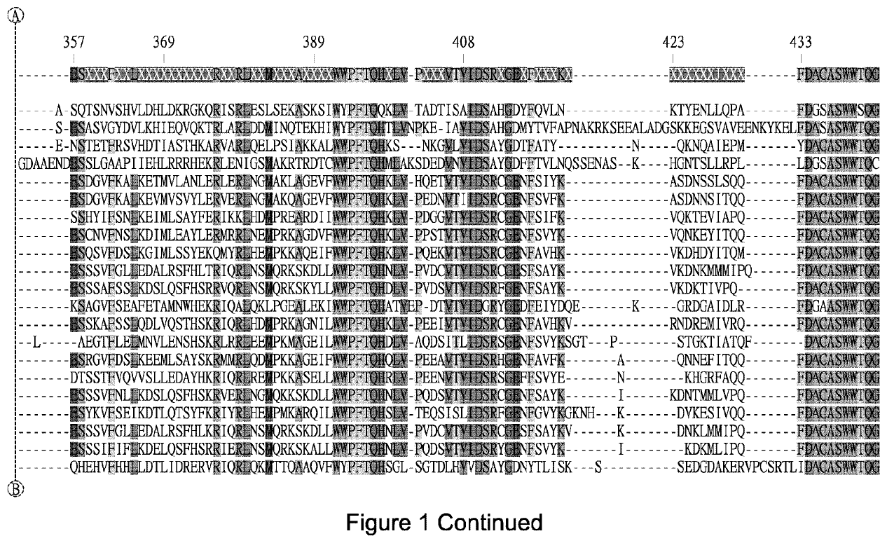

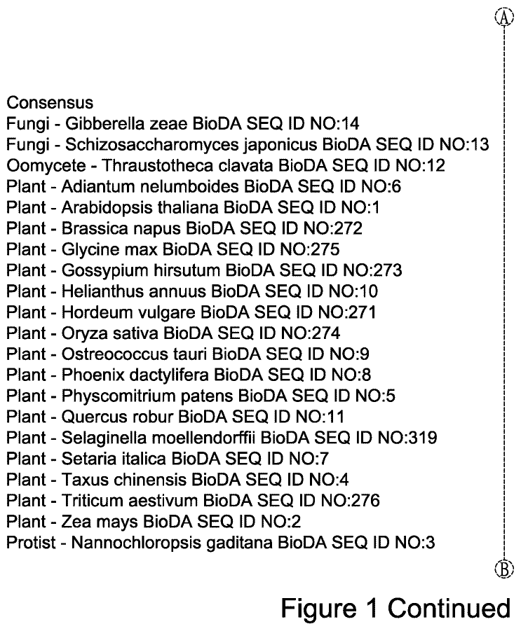

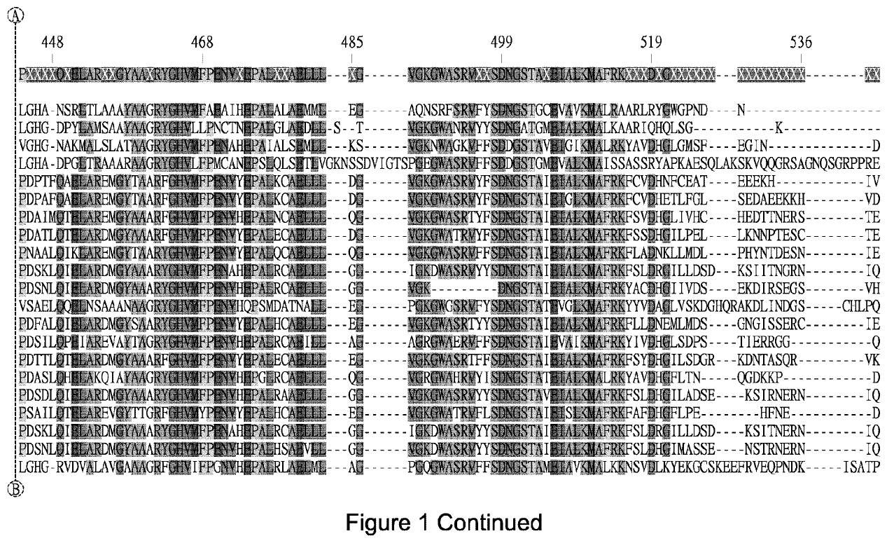

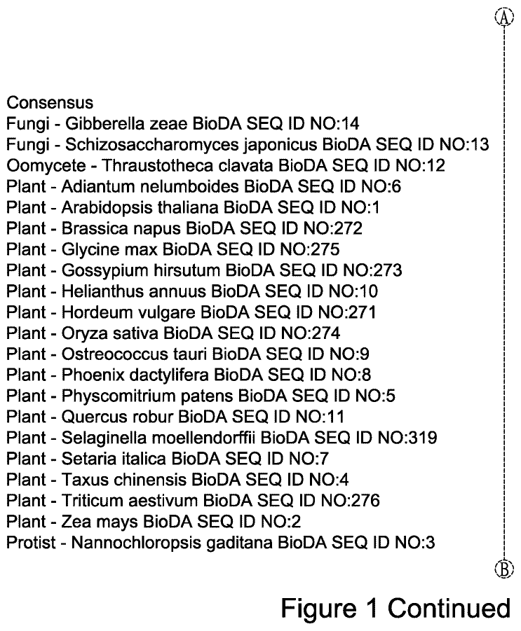

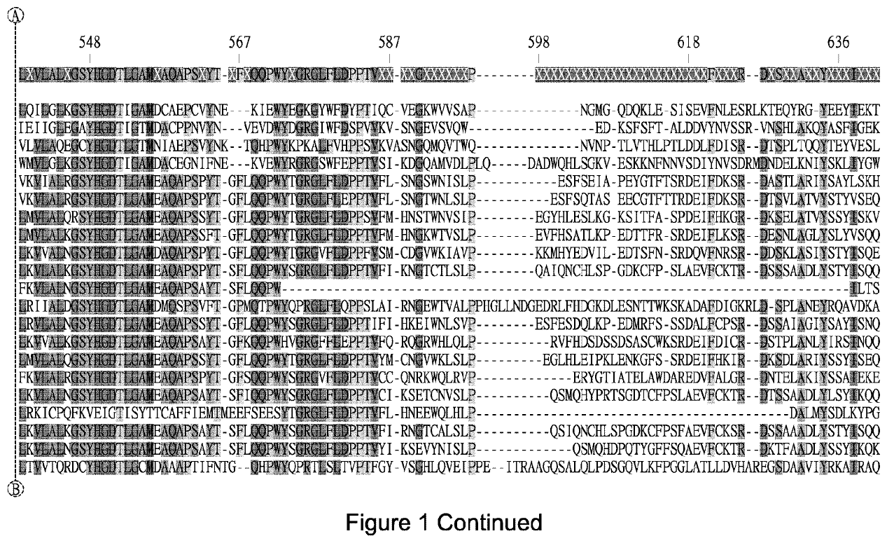

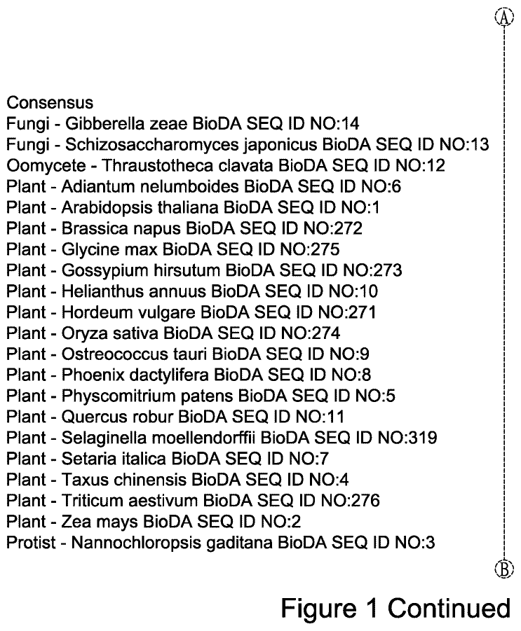

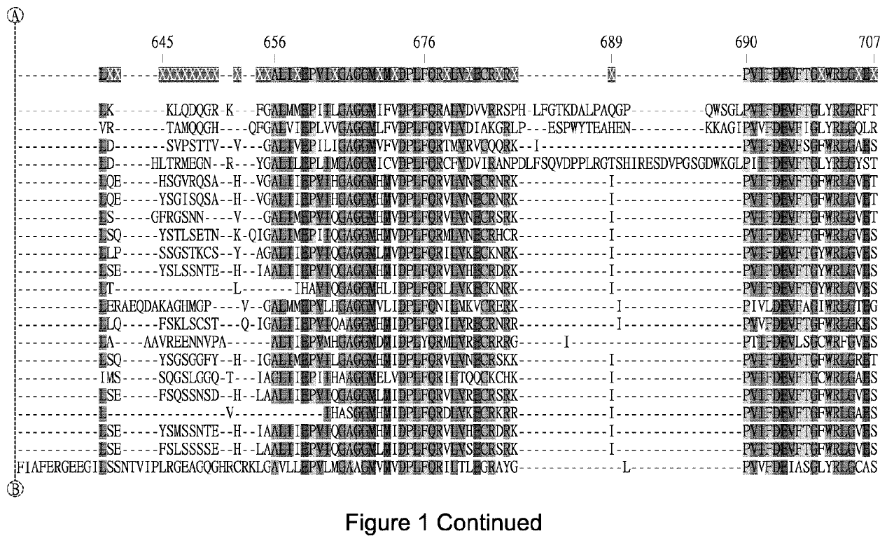

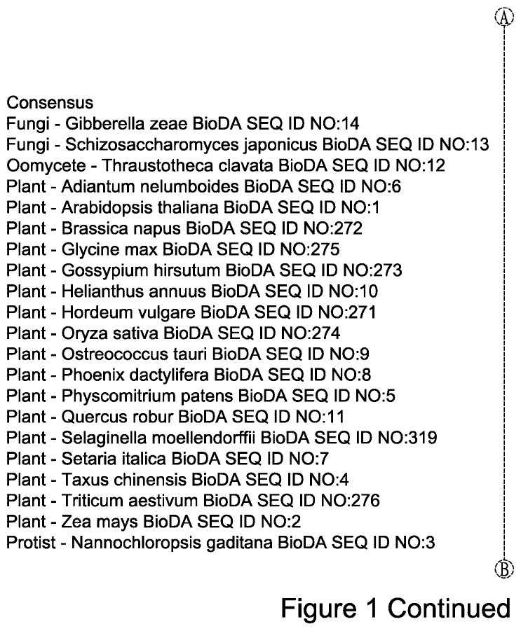

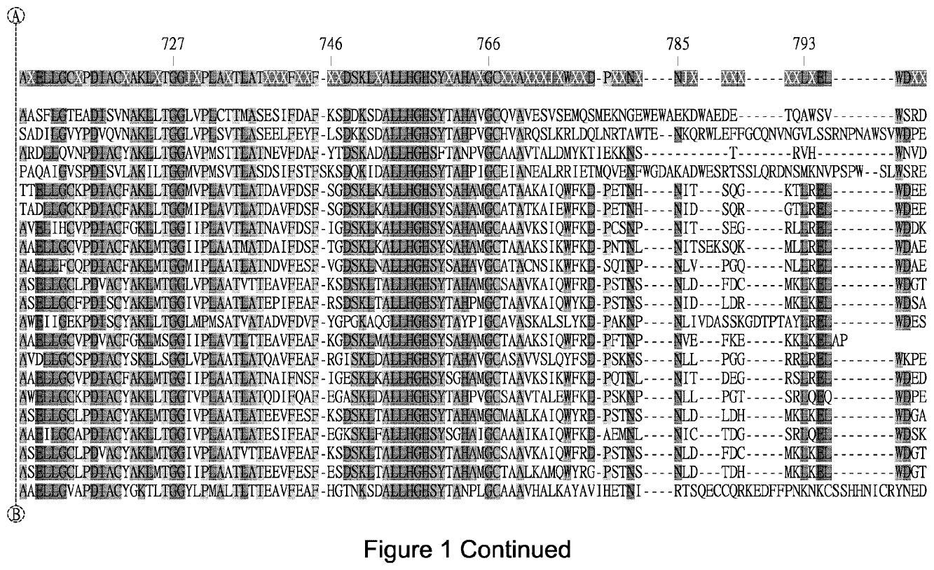

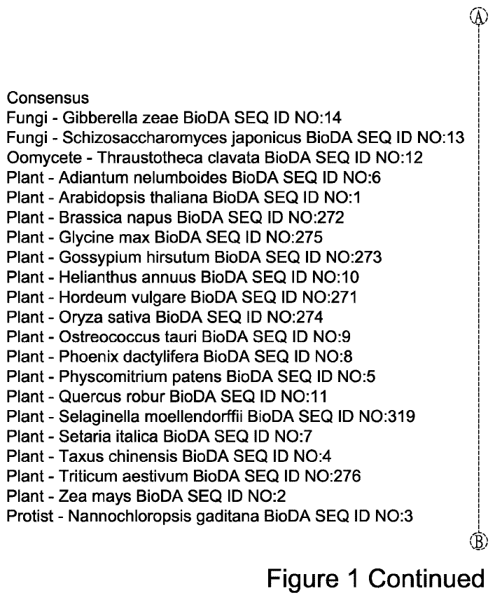

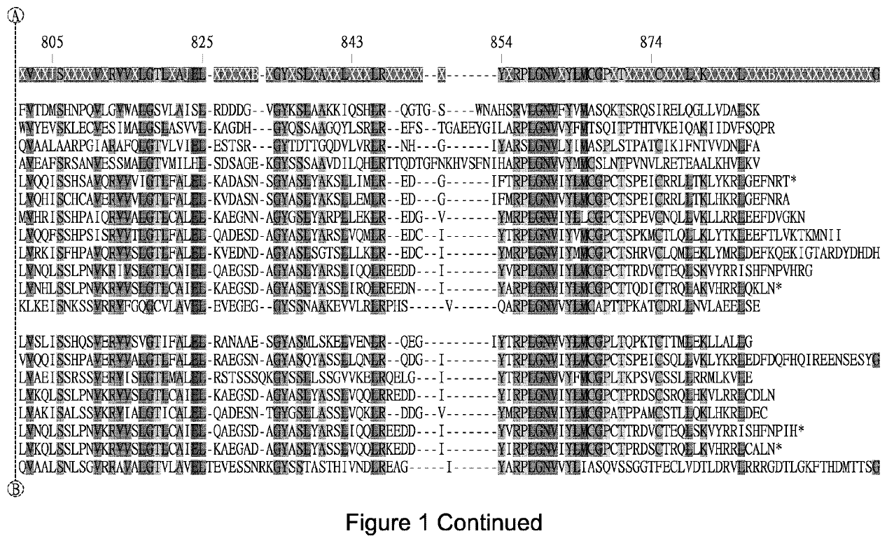

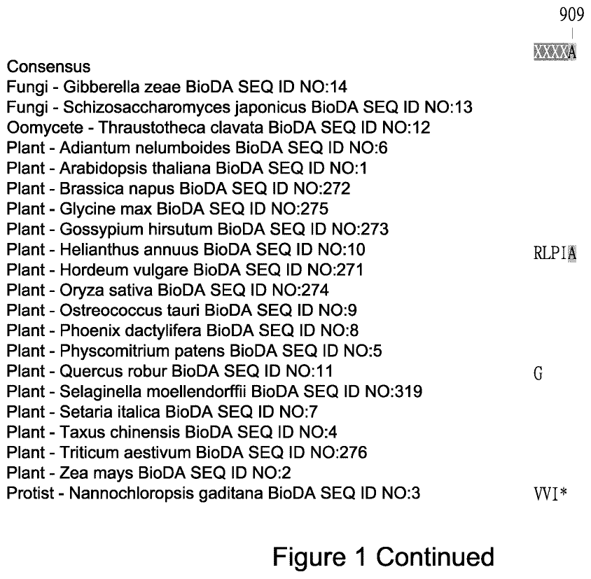

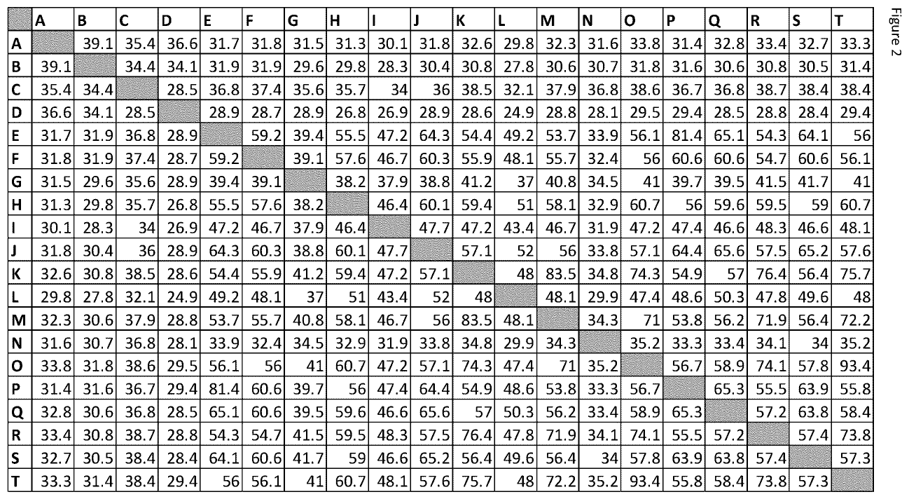

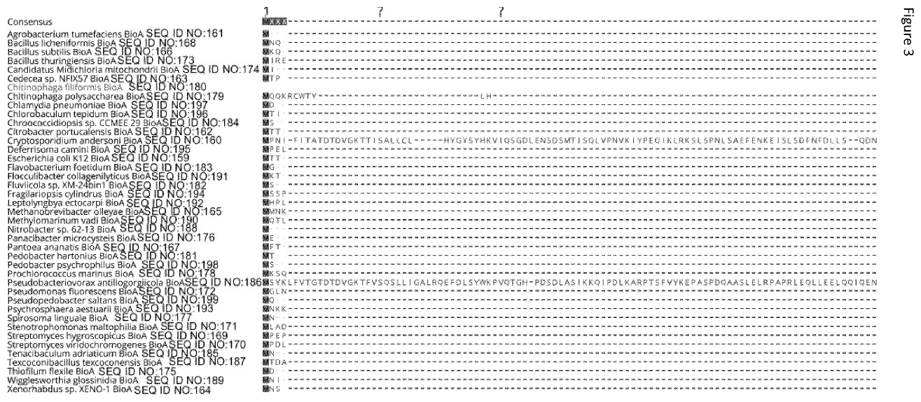

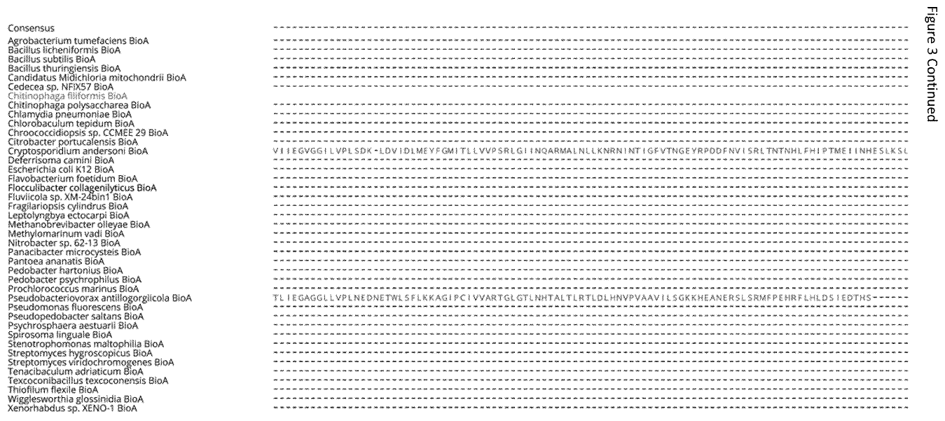

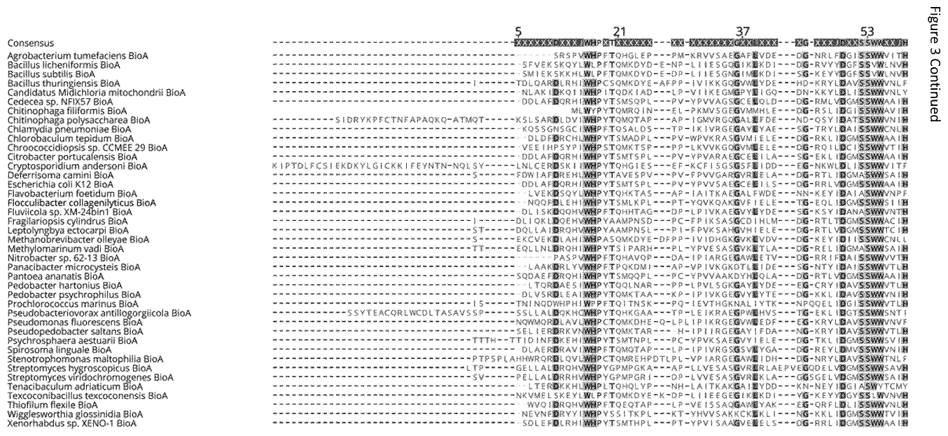

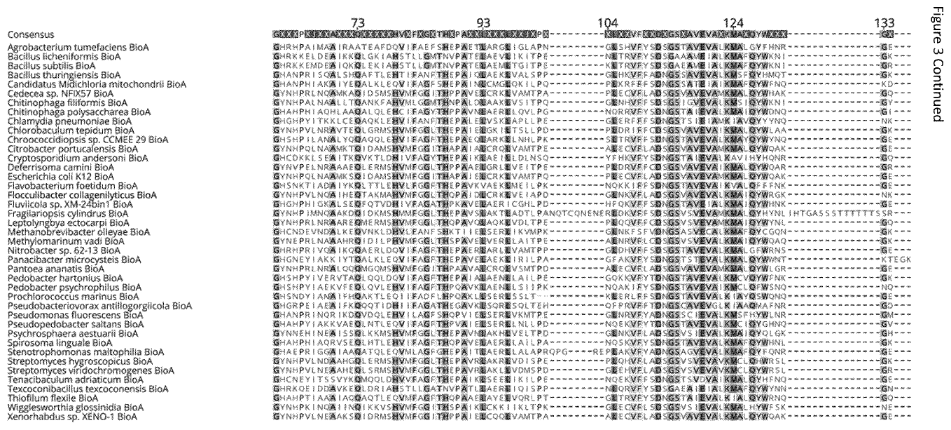

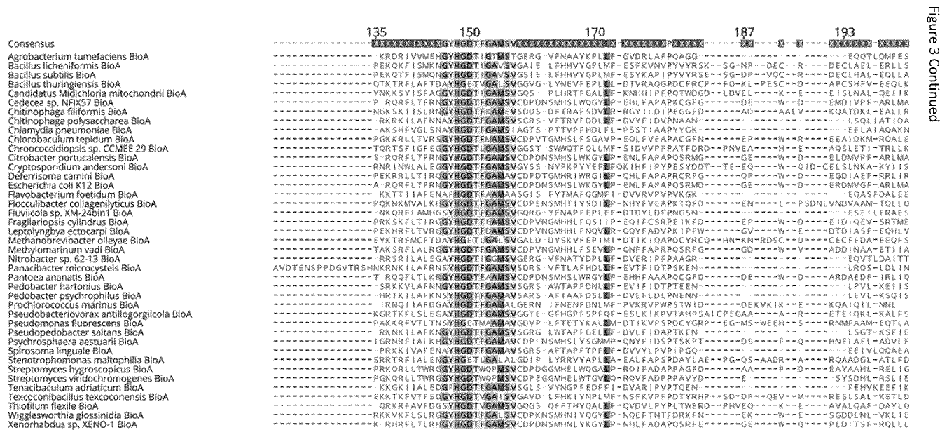

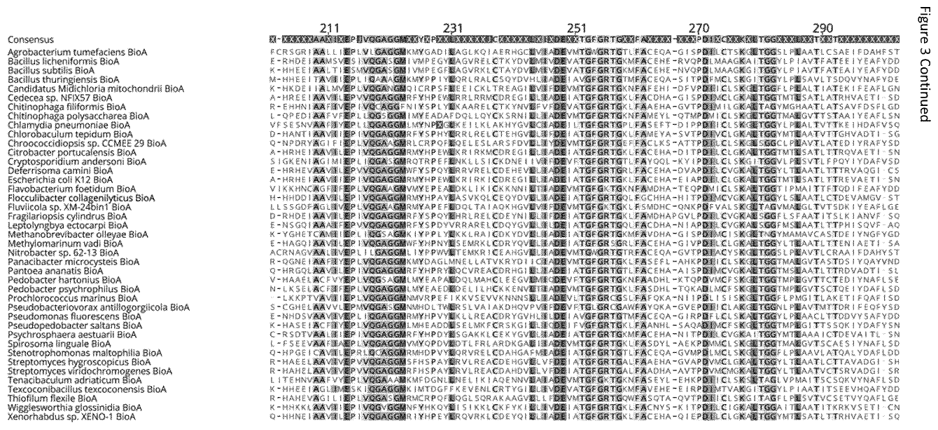

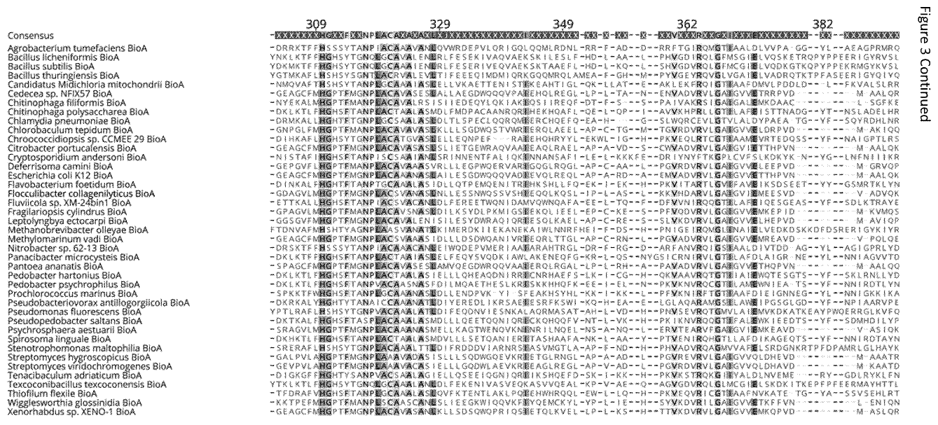

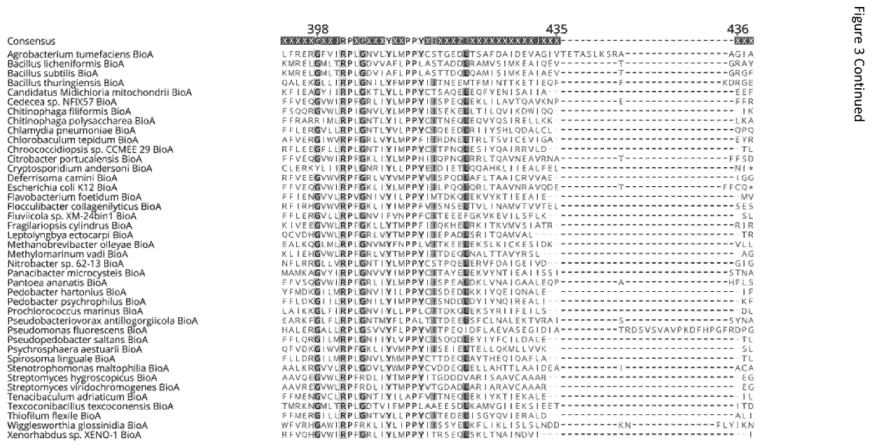

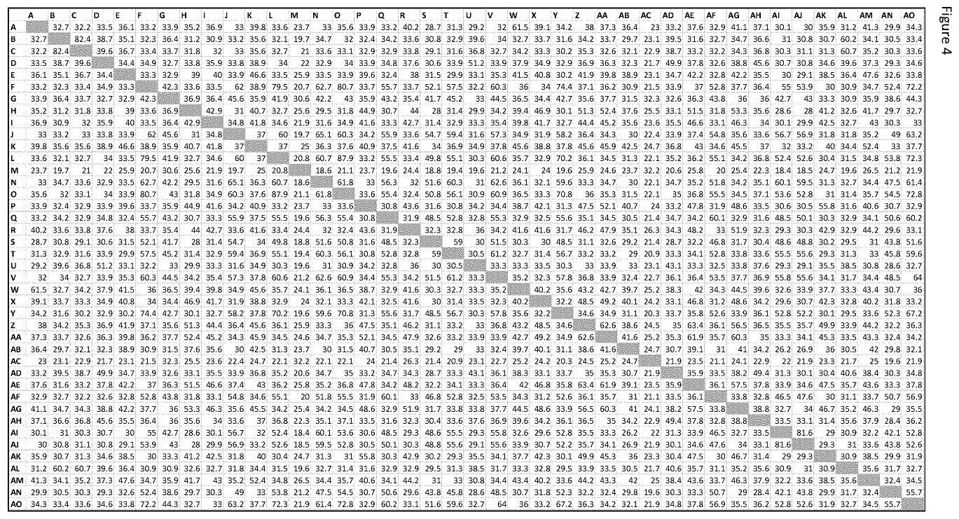

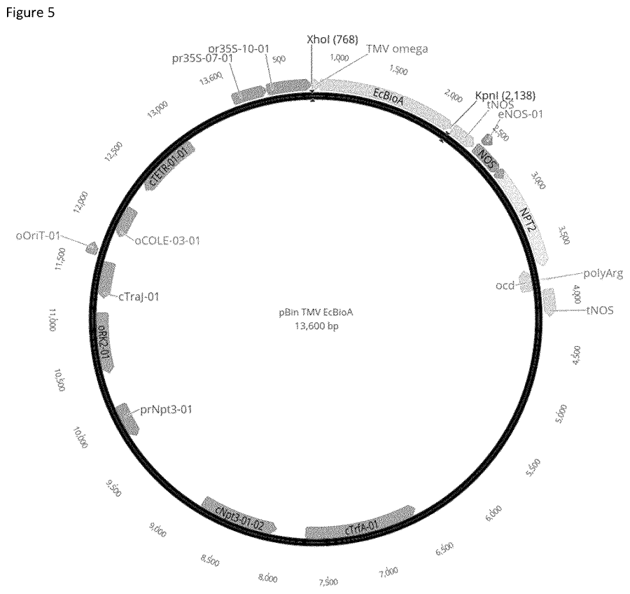

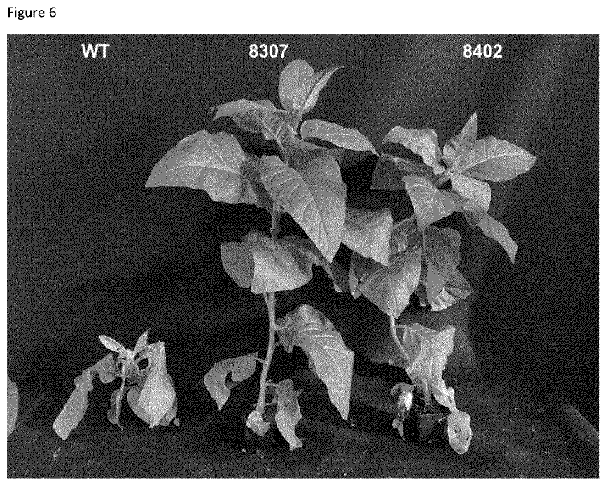

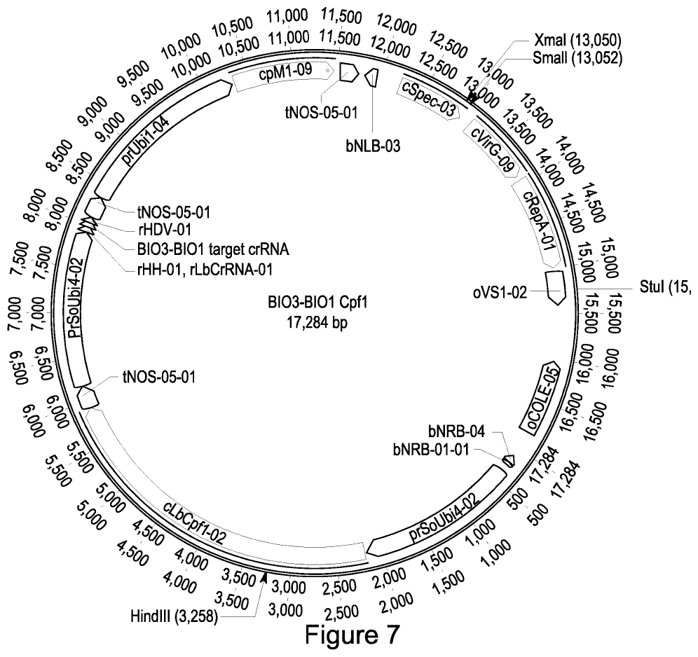

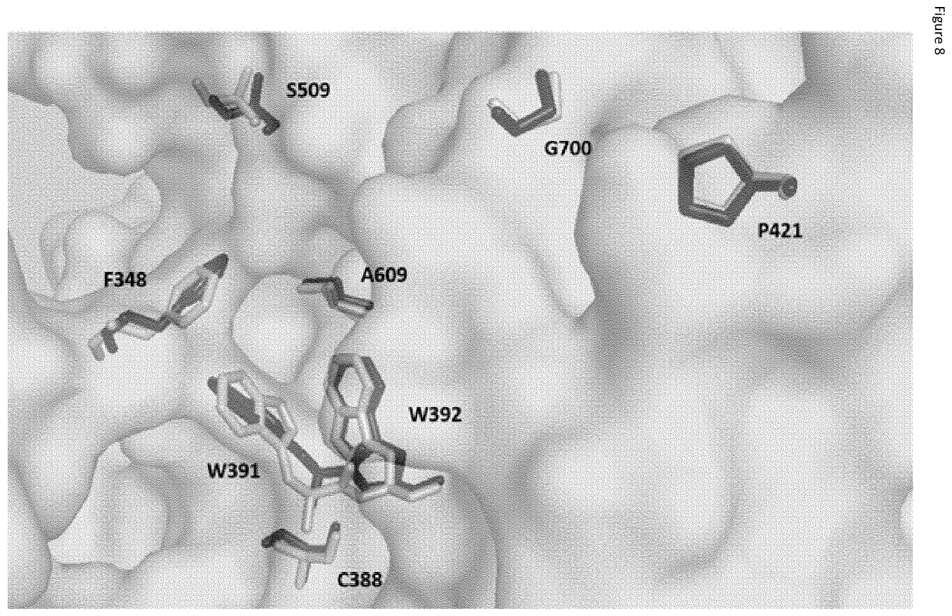

| | |
|---|---|
| A | Giberrella zeae BIO3-BIO1 |
| B | Schizosaccharomyces japonicus BIO3-BIO1 |
| C | Thraustotheca clavata BIO3-BIO1 |
| D | Adiantum nelumboides BIO3-BIO1 |
| E | Arabidopsis thaliana BIO3-BIO1 |
| F | Helianthus annuus BIO3-BIO1 |
| G | Ostreococcus tauri BIO3-BIO1 |
| H | Phoenix dactylifera BIO3-BIO1 |
| I | Physcomitrium patens BIO3-BIO1 |
| G | Quercus robur BIO3-BIO1 |
| K | Setaria italica BIO3-BIO1 |
| L | Taxus chinensis BIO3-BIO1 |
| M | Zea mays BIO3-BIO1 |
| N | Nannochloropsis gaditana BIO3-BIO1 |
| O | Hordeum vulgare BIO3-BIO1 |
| P | Brassica napus BIO3-BIO1 |
| Q | Gossypium hirsutum BIO3-BIO1 |
| R | Oryza sativa BIO3-BIO1 |
| S | Glycine max BIO3-BIO1 |
| T | Triticum aestivum BIO3-BIO1 |

FIG. 3 shows: An alignment of bacterial and archaeal BioA amino acid sequences from Cryptosporidium andersoni (01176813.1) (SEQ ID NO: 160), Escherichia coli K-12 (WP_001295303.1) (SEQ ID NO: 159), Chitinophaga polysaccharea (TWF41277.1) (SEQ ID NO: 179), Fragilariopsis cylindrus (OEU16175) (SEQ ID NO: 194), Spirosoma linguale (ADB41851.1) (SEQ ID NO: 177), Agrobacterium tumefaciens (WP_174070308.1) (SEQ ID NO: 161), Bacillus licheniformis (WP_268389280.1) (SEQ ID NO: 168), Bacillus subtilis (WP_072589144.1) (SEQ ID NO: 166), Candidatus Midichloria mitochondrii (WP_013950979.1) (SEQ ID NO: 174), Cedecea sp. NFIX57 (WP_139827385) (SEQ ID NO: 163), Chitinophaga filiformis (WP_089833099.1) (SEQ ID NO: 180), Chlamydia pneumoniae (WP_035416931.1) (SEQ ID NO: 197), Chlorobaculum tepidum (WP_010931741) (SEQ ID NO: 196), Chroococcidiopsis sp. CCMEE 29 (WP_250122010.1) (SEQ ID NO: 184), Citrobacter portucalensis (WP_236874365) (SEQ ID NO: 162), Deferrisoma camini (WP_025323505) (SEQ ID NO: 195), Flavobacterium foetidum (WP_135222194.1) (SEQ ID NO: 183), Flocculibacter collagenilyticus (WP_199611612) (SEQ ID NO: 191), Leptolyngbya ectocarpi (WP_193993668) (SEQ ID NO: 192), Methanobrevibacter olleyae (WP_234970527) (SEQ ID NO: 165), Methylomarinum vadi (WP_031434384) (SEQ ID NO: 190), Panacibacter microcysteis (WP_196990573.1) (SEQ ID NO: 176), Pantoea ananatis (WP_013025101) (SEQ ID NO: 167), Pedobacter hartonius (WP_090559870.1) (SEQ ID NO: 181), Pedobacter psychrophilus (WP_068823504.1) (SEQ ID NO: 198), Prochlorococcus marinus (WP_011820953.1) (SEQ ID NO: 178), Pseudobacteriovorax antillogorgiicola (WP_132320824.1) (SEQ ID NO: 186), Pseudomonas fluorescens (WP_150601950.1) (SEQ ID NO: 172), Pseudopedobacter saltans (WP_013634352.1) (SEQ ID NO: 199), Psychrosphaera aestuarii (WP_210559569) (SEQ ID NO: 193), Stenotrophomonas maltophilia (WP_262102920.1) (SEQ ID NO: 171), Streptomyces hygroscopicus (WP_244417223.1) (SEQ ID NO: 169), Streptomyces viridochromogenes (WP_004002943.1) (SEQ ID NO: 170), Tenacibaculum adriaticum (WP_148868687.1) (SEQ ID NO: 185), Texcoconibacillus texcoconensis (WP_262102920.1) (SEQ ID NO: 187), Thiofilum flexile (WP_020558297.1) (SEQ ID NO: 175), Xenorhabdus sp. (WP_254851451) (SEQ ID NO: 164), Wigglesworthia glossinidia (BAC24598) (SEQ ID NO: 189), Fluviicola sp. XM-24bin1 (PWL28743.) (SEQ ID NO: 182), Nitrobacter sp. 62-13 (OJU30413.) (SEQ ID NO: 188), and Bacillus thuringiensis (MBE5084136) (SEQ ID NO: 173), generated using Geneious as a global alignment with free end gaps having the following parameters: cost matrix Blosum62, gap open penalty 12, gap extension penalty 3, refinement iterations 2.

FIG. 4 shows: A distance matrix showing the percentage sequence identity between the BioA amino acid sequences of the alignment of FIG. 3, with A to AO defined as follows:

| | |
|---|---|
| A | Agrobacterium tumefaciens BioA |
| B | Bacillus licheniformis BioA |
| C | Bacillus subtilis BioA |
| D | Bacillus thuringiensis BioA |
| E | Candidatus Midichloria mitochondrii BioA |
| F | Cedecea sp. NFIX57 BioA |
| G | Chitinophaga filiformis BioA |
| H | Chitinophaga polysaccharea BioA |
| I | Chlamydia pneumoniae BioA |
| J | Chlorobaculum tepidum BioA |
| K | Chroococcidiopsis sp. CCMEE 29 BioA |
| L | Citrobacter portucalensis BioA |
| M | Cryptosporidium andersoni BioA |
| N | Deferrisoma camini BioA |
| O | Escherichia coli K12 BioA |
| P | Flavobacterium foetidum BioA |
| Q | Flocculibacter collagenilyticus BioA |
| R | Fluviicola sp. XM-24bin1 BioA |
| S | Fragilariopsis cylindrus BioA |
| T | Leptolyngbya ectocarpi BioA |
| U | Methanobrevibacter olleyae BioA |
| V | Methylomarinum vadi BioA |
| W | Nitrobacter sp. 62-13 BioA |
| X | Panacibacter microcysteis BioA |
| Y | Pantoea ananatis BioA |
| Z | Pedobacter hartonius BioA |
| AA | Pedobacter psychrophilus BioA |
| AB | Prochlorococcus marinus BioA |
| AC | Pseudobacteriovorax antillogorgiicola BioA |
| AD | Pseudomonas fluorescens BioA |
| AE | Pseudopedobacter saltans BioA |
| AF | Psychrosphaera aestuarii BioA |
| AG | Spirosoma linguale BioA |
| AH | Stenotrophomonas maltophilia BioA |
| AI | Streptomyces hygroscopicus BioA |
| AJ | Streptomyces viridochromogenes BioA |
| AK | Tenacibaculum adriaticum BioA |
| AL | Texcoconibacillus texcoconensis BioA |
| AM | Thiofilum flexile BioA |
| AN | Wigglesworthia glossinidia BioA |
| AO | Xenorhabdus sp. XENO-1 BioA |

FIG. 5 shows: a representation of a binary vector (pBin) for tobacco transformation, conferring resistance to biotin pathway-inhibiting herbicides comprising a tobacco codon optimized E. coli BioA gene (SEQ ID NO:206. This binary vector also contains an NPT2 selectable marker for kanamycin selection.

FIG. 6 shows: a photograph of representative wild type (WT) tobacco plants compared to modified tobacco plants comprising Event 8307 transformed with pBin TMV EcBioA and Event 8402 transformed with pBin TMV MTP EcBioA, after treatment with a herbicide compound L (600 gai/ha) which inhibits the biotin pathway. The wild type plant shows significantly stunted growth compared to plant 8307 and 8402.

FIG. 7 shows: a map of an exemplary genome editing vector construct comprising Cpf1 and at least one guide RNA that could be used to edit a BIO3-BIO1 gene within a maize plant and provide resistance to biotin pathway inhibiting herbicides.

FIG. 8 shows: Part of the Arabidopsis thaliana BioDA crystal structure with selected amino acids highlighted in stick visualisation. The Ostreococcus tauri BioDA was modelled in AlphaFold and structurally aligned to the crystal structure of Arabidopsis thaliana BioDA and selected amino acids that align in primary sequence alignments are highlighted as stick visualisations from Arabidopsis thaliana (dark grey) and Ostreococcus tauri (light grey). The highlighted Arabidopsis thaliana amino acids are labelled.

### EXAMPLES

Example 1—BIO3-BIO1 (BioDA) sequences and expression in tobacco plants Arabidopsis thaliana BIO3-BIO1 or orthologues of this, for example SEQ ID NOs: 1 to 14, 271-276, are expressed in transgenic tobacco. DNA sequences that encode these polypeptides (optimized for tobacco or, optionally, codon optimized according to a target crop such as soybean or corn) are prepared synthetically. Each sequence is designed to include a 5′ fusion with TMV omega 5′ leader sequence and such that they are flanked at the 5′ end with Xhol and at the 3′ end with Kpnl to facilitate direct cloning into a suitable binary vector for Agrobacterium-based plant transformation.

In one example, the expression cassette, comprising the TMV omega 5′ leader and a BIO3-BIO1encoding gene of interest is excised using Xhol/Kpnl and cloned into similarly digested pBIN 19 (Bevan, Nucleic Acids Res. 12:8711-8721 (1984) to create a binary vector, e.g. the vector of FIG. 5, (SEQ ID NO: 206) behind a double enhanced 35S promoter ahead of a NOS 3′ transcription terminator and then transformed into E. coli DH5 alpha competent cells. DNA recovered from the E. coli is used to transform Agrobacterium tumefaciens LBA4404, and the transformed bacteria are selected on media contain rifampicin and kanamycin. Tobacco tissue is subjected to Agrobacterium-mediated transformation using methods well described in the art or as described herein. For example, a master plate of Agrobacterium tumefaciens containing the BIO3-BIO1 expressing binary vector is used to inoculate 10 ml LB (L broth) containing 100 mg/l rifampicin plus 50 mg/l kanamycin using a single bacterial colony. This is incubated overnight at 28° C. shaking at 200 rpm. This entire overnight culture is used to inoculate a 50 ml volume of LB containing the same antibiotics. Again, this is cultured overnight at 28° C. shaking at 200 rpm. The Agrobacterium cells are pelleted by centrifuging at 3000 rpm for 15 minutes and then re-suspended in MS (Murashige and Skoog) medium containing 30 g/l sucrose, pH 5.9 to an OD (600 nM)=0.6. This suspension is dispensed in 25 ml aliquots into petri dishes.

Clonally micro-propagated tobacco shoot cultures are used to excise young (not yet fully expanded) leaves. The mid rib and outer leaf margins are removed and discarded, and the remaining lamina cut into 1 cm squares. These are transferred to the Agrobacterium suspension for 20 minutes. Explants are then removed, dabbed on sterile filter paper to remove excess suspension, then transferred onto solid NBM medium (MS medium containing 30 g/l sucrose, 1 mg/l BAP (benzylaminopurine) and 0.1 mg/l NAA (napthalene acetic acid) at pH 5.9 and solidified with 8 g/l Plantagar), with the abaxial surface of each explant in contact with the medium. Approximately 7 explants are transferred per plate, which are then sealed and maintained in a lit incubator at 25° C. for a 16 hour photoperiod for 3 days.

Explants are then transferred onto NBM medium containing 100 mg/l kanamycin plus antibiotics to prevent further growth of Agrobacterium (200 mg/l timentin with 250 mg/l carbenicillin). Further subculture onto this same medium was then performed every 2 weeks.

As shoots start to regenerate from the callusing leaf explants, these are removed to Shoot elongation medium (MS medium, 30 g/l sucrose, 8 g/l Plantagar, 100 mg/l kanamycin, 200 mg/l timentin, 250 mg/l carbenicillin, pH 5.9). Stable transgenic plants readily root within 2 weeks. To provide multiple plants per event to ultimately allow more than one herbicide test per transgenic plant, all rooting shoots are micropropagated to generate 3 or more rooted clones.

Putative transgenic plants that are rooting and showing vigorous shoot growth on the medium incorporating kanamycin are analysed by PCR using primers that amplified a 500 bp fragment specific to the BIO3-BO1transgene of interest. Evaluation of this same primer set on untransformed tobacco showed conclusively that these primers would not amplify any sequences from the native tobacco genome.

Transformed shoots are divided into 2 or 3 clones and regenerated from kanamycin resistant callus. Shoots are rooted on MS agar containing kanamycin. Surviving rooted explants are re-rooted to provide approximately 40-50 kanamycin resistant and PCR positive events from each event.

Once rooted, plantlets are transferred from agar and potted into 50% peat, 50% John Innes Soil No. 3 with slow-release fertilizer in 3 inch round pots and left regularly watered to establish for 8-12d in the glass house. Glasshouse conditions are about 24-27° C. day; 18-21° C. night and approximately a 14 h photoperiod. Humidity is adjusted to ~65% and light levels used are up to 2000 mmol/m² at bench level.

Transgenic populations of about forty tobacco plants that comprise a gene encoding a full length BIO3-BO1gene (e.g. SEQ ID NO: 1 (Arabidopsis thaliana BIO3-BIO1) or 2 to 14) are thus produced. Plants are selected on the basis of similar size from each population and ELISA or Mass Western tests are carried out to monitor protein transgenic BIO3-BIO1 expression levels. The highest expressing TO lines are selected to be taken forward to self and to generate T1 seed and T2 lines and seed in the normal way. Seeds from the highest expressing lines are tested for germination on agar plates containing a range of concentrations of BIO3-BIO1-inhibiting herbicides as taught for example herein and resistant plant lines selected as showing the least damage to root or leaf growth and morphology at the highest concentrations of herbicides. Resistant plant lines exhibit a dose response in respect of herbicidal damage by BIO3-BIO1 inhibitors that is shifted to the right in comparison with similarly grown and treated wild type and null segregant plants.

### Example 2—Assay of Herbicide Tolerance in Transgenic Tobacco Plants Expressing Heterologous BIO3-BIO1 (BioDA

In order to determine if the overexpression of the genes detailed in Example 1 confer resistance to BIO3-BIO1 inhibiting compounds when expressed in plants, GM tobacco lines are to be produced and tested with BIO3-BIO1 inhibiting compounds. Transgenic tobacco plants expressing the Arabidopsis thaliana BIO3-BIO1 gene (Seq ID 1), the Zea mays BIO3-BIO1 gene (Seq ID 2), or the Nannochloropsis gaditana BIO3-BIO1 gene (Seq ID 3).

Populations of transgenic tobacco comprising 20-30 transgenic events per plant transformation constructs were generated as described in Example 1. These lines were clonally propagated and 1 clone per event was sprayed with 600 g ai/ha compound L or A+0.2% Genapol X080 with a spray volume of 200 L/Ha (“g ai/ha” refers to grams of active compound per hectare).

Herbicidal damage was visually assessed across the population and a herbicide damage score given at 7, 14 and 21 days. A score of 0 indicates no visible damage or stunting whereas a score of 100 indicates a complete death of the plant. 20 transgenic events (i.e. individual transgenic plants) from each GM tobacco line were transplanted to soil 1 week prior to spraying with BIO3-BIO1inhibiting compounds. WT tobacco was included as a control. Table 2 below shows the treatment regimens used.

TABLE 2
treatment regimens for testing BIO3-BIO1 inhibitor resistance

| Treatment | | Application rate | Formulation |
|---|---|---|---|
| no | Compound | (g ai/ha) | and Adjuvant |
| 1 | L | 600 | Genapol X080 |
| | | | 0.2% |
| 2 | A | 600 | Genapol X080 |
| | | | 0.2% |

TABLE 3
Plants transformed with BIO3-BIO1 genes.

| | | | Protein | Nucleic |
|---|---|---|---|---|
| DNA | | | SEQ | acid SEQ |
| ID | Name | Gene background | ID NO | ID NO |
| D4556 | pCMP ZmBioDA | Zea mays | 2 | 157 |
| D4557 | pCMP AtBioDA | Arabidopsis thaliana | 1 | 156 |
| D4558 | pCMP NgBioDA | Nannochloropsis | 3 | 158 |
| | | gaditana | | |
| D4612 | pCMP OtBioDA | Ostreococcus tauri | 9 | 349 |

The results are shown in Tables 4 to 7 and Table 15 below. A control population of wild-type Samsun tobacco was assessed for herbicide damage with result shown in table 2. The wild-type Samsun plants show significant damage at the day 14 and 21 assessment time. The values in Tables 2 to 6 below show the percentage damage scores for populations of 20 plants expressing either the Zea mays BIO3-BIO1 gene (SEQ ID NO. 2), the Arabidopsis BIO3-BIO1 gene (SEQ ID NO. 1) or the Nannochloropsis gaditana BIO3-BIOlgene (SEQ ID NO. 3) at day 7, 14 and 21 post treatment It is clear that the overexpression of the Zea mays BIO3-BIO1, Arabidopsis thaliana BIO3-BIO1 or Nannochloropsis gaditana BIO3-BIO1 genes significantly increases tolerance to herbicide with some events showing no damage at all.

TABLE 4
results of percentage of damage to WT tobacco plants

| | Wildtype Samsun tobacco | | | | | |
|---|---|---|---|---|---|---|
| | Compound L (600 gai/ha) | | | Compound A (600 gai/ha) | | |
| WT | 7 DAT | 14 DAT | 21 DAT | 7 DAT | 14 DAT | 21 DAT |
| 1 | 85 | 93 | 95 | 65 | 65 | 75 |
| 2 | 85 | 93 | 95 | 65 | 70 | 75 |
| 3 | 85 | 90 | 90 | 55 | 70 | 70 |
| 4 | 90 | 95 | 97 | 60 | 75 | 80 |
| 5 | 85 | 90 | 95 | 55 | 70 | 75 |
| 6 | 85 | 90 | 95 | 60 | 75 | 80 |
| 7 | 93 | 95 | 98 | 30 | 65 | 85 |
| 8 | 85 | 90 | 90 | 65 | 75 | 80 |

TABLE 5
results of percentage of damage to D4556
(Zea mays BIO3-BIO1) containing tobacco plants

| | pCMP ZmBioDA | | | | | |
|---|---|---|---|---|---|---|
| | Compound L (600 gai/ha) | | | Compound A (600 gai/ha) | | |
| D4556 | 7 DAT | 14 DAT | 21 DAT | 7 DAT | 14 DAT | 21 DAT |
| 8554 | 80 | 90 | 95 | 25 | 65 | 60 |
| 8556 | 70 | 40 | 20 | 40 | 60 | 55 |
| 8557 | 85 | 90 | 90 | 40 | 65 | 55 |
| 8562 | 85 | 90 | 90 | 50 | 70 | 65 |
| 8574 | 80 | 75 | 50 | 30 | 60 | 45 |
| 8575 | 85 | 90 | 90 | 45 | 70 | 65 |
| 8580 | 85 | 90 | 90 | 45 | 60 | 65 |
| 8584 | 75 | 75 | 55 | 35 | 40 | 30 |
| 8586 | 90 | 90 | 95 | 50 | 75 | 75 |
| 8590 | 85 | 90 | 90 | 50 | 75 | 75 |
| 8591 | 85 | 90 | 90 | 55 | 75 | 75 |
| 8596 | 75 | 80 | 70 | 55 | 75 | 80 |
| 8601 | 85 | 90 | 90 | 50 | 70 | 75 |
| 8603 | 80 | 90 | 90 | 55 | 70 | 75 |
| 8604 | 85 | 90 | 90 | 55 | 70 | 75 |
| 8606 | 80 | 90 | 85 | 60 | 75 | 70 |
| 8607 | 85 | 90 | 90 | 45 | 65 | 50 |
| 8609 | 80 | 90 | 90 | 40 | 60 | 40 |
| 8610 | 85 | 90 | 90 | 40 | 65 | 45 |
| 8626 | 85 | 90 | 85 | 50 | 70 | 70 |

TABLE 6
results of percentage of damage to D4557 (Arabidopsis thaliana
BIO3-BIO1) containing tobacco plants

| | pCMP AtBioDA | | | | | |
|---|---|---|---|---|---|---|
| | Compound L (600 gai/ha) | | | Compound A (600 gai/ha) | | |
| D4557 | 7 DAT | 14 DAT | 21 DAT | 7 DAT | 14 DAT | 21 DAT |
| 8643 | 85 | 75 | 65 | 5 | 5 | 0 |
| 8645 | 85 | 75 | 40 | 0 | 0 | 0 |
| 8646 | 85 | 55 | 10 | 3 | 2 | 0 |
| 8655 | 90 | 85 | 75 | 35 | 60 | 40 |
| 8660 | 85 | 85 | 80 | 10 | 25 | 30 |
| 8661 | 85 | 90 | 90 | 50 | 70 | 75 |
| 8667 | 9 | 80 | 80 | 0 | 0 | 0 |
| 8671 | 85 | 80 | 55 | 20 | 20 | 8 |
| 8673 | 80 | 60 | 25 | 10 | 5 | 5 |
| 8674 | 80 | 65 | 25 | 35 | 15 | 15 |
| 8679 | 85 | 50 | 35 | 15 | 5 | 2 |
| 8684 | 45 | 15 | 10 | 0 | 0 | 0 |
| 8690 | 75 | 75 | 80 | 0 | 5 | 0 |
| 8697 | 80 | 85 | 80 | 20 | 55 | 40 |
| 8701 | 80 | 75 | 50 | 5 | 0 | 0 |
| 8709 | 90 | 90 | 85 | 5 | 0 | 0 |
| 8712 | 80 | 80 | 80 | 5 | 15 | 0 |
| 8713 | 70 | 30 | 10 | 10 | 5 | 0 |
| 8714 | 75 | 70 | 40 | 0 | 2 | 2 |
| 8716 | 80 | 90 | 90 | 35 | 70 | 75 |

TABLE 7
results of percentage of damage to D4558 (Nannochloropsis gaditana
BIO3-BIO1) containing tobacco plants

| | pCMP NgBioDA | | | | | |
|---|---|---|---|---|---|---|
| | Compound L (600 gai/ha) | | | Compound A (600 gai/ha) | | |
| D4558 | 7 DAT | 14 DAT | 21 DAT | 7 DAT | 14 DAT | 21DAT |
| 8727 | 70 | 30 | 10 | 5 | 0 | 0 |
| 8735 | 60 | 30 | 5 | 0 | 0 | 0 |
| 8737 | 75 | 45 | 5 | 3 | 0 | 0 |
| 8738 | 95 | 90 | 80 | 5 | 0 | 60 |
| 8744 | 95 | 98 | 98 | 50 | 75 | 80 |
| 8749 | 85 | 65 | 20 | 10 | 2 | 0 |
| 8750 | 85 | 90 | 90 | 50 | 80 | 85 |
| 8757 | 90 | 90 | 80 | 15 | 10 | 5 |
| 8759 | 85 | 90 | 90 | 60 | 75 | 80 |
| 8760 | 70 | 50 | 15 | 0 | 0 | 0 |
| 8762 | 75 | 40 | 5 | 35 | 35 | 8 |
| 8765 | 80 | 55 | 20 | 40 | 0 | 20 |
| 8767 | 85 | 90 | 95 | 65 | 80 | 90 |
| 8781 | 90 | 95 | 96 | 75 | 85 | 80 |
| 8785 | 75 | 55 | 10 | 20 | 0 | 0 |
| 8789 | 90 | 80 | 65 | 0 | 0 | 0 |
| 8794 | 80 | 50 | 10 | 0 | 0 | 0 |
| 8795 | 85 | 90 | 90 | 40 | 65 | 65 |
| 8796 | 70 | 35 | 5 | 35 | 0 | 0 |
| 8799 | 75 | 55 | 10 | 5 | 0 | 2 |

Results of percentage of damage to 04612 (Ostreococcus tauri BIO3-BIO1) containing tobacco plants is provided in Table 15 in the “TABLES” section below.

### Example 3—BioA Sequences and Expression in Tobacco Plants

Escherichia coli BioA or orthologues of this, for example SEQ ID NOs: 159-199, are expressed in transgenic tobacco. DNA sequences that encode these polypeptides (optimized for tobacco or, optionally, codon optimized according to a target crop such as soybean or corn) are prepared synthetically. Each sequence is designed to include a 5′ fusion with TMV omega 5′ leader sequence and such that they are flanked at the 5′ end with Xhol and at the 3′ end with Kpnl to facilitate direct cloning into a suitable binary vector for Agrobacterium-based plant transformation.

In one example, the expression cassette, comprising the TMV omega 5′ leader and a BioA encoding gene of interest is excised using Xhol/Kpnl and cloned into similarly digested pBIN 19 (Bevan, Nucleic Acids Res. 12:8711-8721 (1984) behind a double enhanced 35S promoter ahead of a NOS 3′ transcription terminator and then transformed into E. coli DH5 alpha competent cells. In another example, the BioA gene of interest is cloned into pCMP behind the Cestrum yellow leaf curling virus (CmYLCV) promoter and ahead of a NOS 3′ transcription terminator and then transformed into E. coli DH5 alpha competent cells. DNA recovered from the E. coli is used to transform Agrobacterium tumefaciens LBA4404, and the transformed bacteria are selected on media contain rifampicin and kanamycin. Tobacco tissue is subjected to Agrobacterium-mediated transformation using methods well described in the art or as described herein. For example, a master plate of Agrobacterium tumefaciens containing the BioA expressing binary vector is used to inoculate 10 ml LB (L broth) containing 100 mg/l rifampicin plus 50 mg/l kanamycin using a single bacterial colony. This is incubated overnight at 28° C. shaking at 200 rpm. This entire overnight culture is used to inoculate a 50 ml volume of LB containing the same antibiotics. Again, this is cultured overnight at 28° C. shaking at 200 rpm. The Agrobacterium cells are pelleted by centrifuging at 3000 rpm for 15 minutes and then re-suspended in MS (Murashige and Skoog) medium containing 30 g/l sucrose, pH 5.9 to an OD (600 nM)=0.6. This suspension is dispensed in 25 ml aliquots into petri dishes.

Clonally micro-propagated tobacco shoot cultures are used to excise young (not yet fully expanded) leaves. The mid rib and outer leaf margins are removed and discarded, and the remaining lamina cut into 1 cm squares. These are transferred to the Agrobacterium suspension for 20 minutes. Explants are then removed, dabbed on sterile filter paper to remove excess suspension, then transferred onto solid NBM medium (MS medium containing 30 g/l sucrose, 1 mg/l BAP (benzylaminopurine) and 0.1 mg/l NAA (napthalene acetic acid) at pH 5.9 and solidified with 8 g/l Plantagar), with the abaxial surface of each explant in contact with the medium.

Approximately 7 explants are transferred per plate, which are then sealed and maintained in a lit incubator at 25° C. for a 16 hour photoperiod for 3 days.

Explants are then transferred onto NBM medium containing 100 mg/l kanamycin plus antibiotics to prevent further growth of Agrobacterium (200 mg/l timentin with 250 mg/l carbenicillin). Further subculture onto this same medium was then performed every 2 weeks.

As shoots start to regenerate from the callusing leaf explants, these are removed to Shoot elongation medium (MS medium, 30 g/l sucrose, 8 g/l Plantagar, 100 mg/l kanamycin, 200 mg/l timentin, 250 mg/l carbenicillin, pH 5.9). Stable transgenic plants readily root within 2 weeks. To provide multiple plants per event to ultimately allow more than one herbicide test per transgenic plant, all rooting shoots are micropropagated to generate 3 or more rooted clones.

Putative transgenic plants that are rooting and showing vigorous shoot growth on the medium incorporating kanamycin are analysed by PCR using primers that amplified a 500 bp fragment specific to the BioA transgene of interest. Evaluation of this same primer set on untransformed tobacco showed conclusively that these primers would not amplify any sequences from the native tobacco genome.

Transformed shoots are divided into 2 or 3 clones and regenerated from kanamycin resistant callus. Shoots are rooted on MS agar containing kanamycin. Surviving rooted explants are re-rooted to provide approximately 40-50 kanamycin resistant and PCR positive events from each event.

Once rooted, plantlets are transferred from agar and potted into 50% peat, 50% John Innes Soil No. 3 with slow-release fertilizer in 3 inch round pots and left regularly watered to establish for 8-12d in the glass house. Glasshouse conditions are about 24-27° C. day; 18-21° C. night and approximately a 14 h photoperiod. Humidity is adjusted to ~65% and light levels used are up to 2000 mmol/m² at bench level.

Transgenic populations of about forty tobacco plants that comprise a gene encoding a full length BioA gene (e.g. SEQ ID NO: 159 (Amino acid) and 202 and 203 (nucleic acid) (E coli BioA) or 160-199 (amino acids) and 325 to 348 (nucleic acids) are thus produced. Plants are selected on the basis of similar size from each population and ELISA or Mass Western tests are carried out to monitor protein transgenic BioA expression levels. The highest expressing TO lines are selected to be taken forward to self and to generate T1 seed and T2 lines and seed in the normal way. Seeds from the highest expressing lines are tested for germination on agar plates containing a range of concentrations of BIO3-BIO1-inhibiting herbicides as taught for example herein and resistant plant lines selected as showing the least damage to root or leaf growth and morphology at the highest concentrations of herbicides. Resistant plant lines exhibit a dose response in respect of herbicidal damage by BIO3-BIO1 inhibitors that is shifted to the right in comparison with similarly grown and treated wild type and null segregant plants.

### Example 4—Assay of Herbicide Tolerance in Transgenic Tobacco Plants Expressing Bacterial BioA

In order to determine if the overexpressed gene detailed in Example 3 confers resistance to BIO3-BIO1 inhibiting compounds when expressed in plants, GM tobacco lines were produced and tested with BIO3-BIO1 inhibiting compounds. Transgenic tobacco plants expressing the Escherichia coli BioA gene with (SEQ ID NO:203) and without a mitochondrial signal peptide (SEQ ID NO: 202).

Populations of transgenic tobacco comprising 20-30 transgenic events per plant transformation constructs were generated as described in Example 1. These lines were clonally propagated and 1 clone per event was sprayed with 600 g ai/ha compound L or A+0.2% Genapol X080 with a spray volume of 200 L/Ha (“g ai/ha” refers to grams of active compound per hectare).

Herbicidal damage was visually assessed across the population and a herbicide damage score given at 7 and 14 days. A score of 0 indicates no visible damage or stunting whereas a score of 100 indicates a complete death of the plant. 20 transgenic events (i.e. individual transgenic plants) from each GM tobacco line were transplanted to soil 1 week prior to spraying with BIO3-BIO1 inhibiting compounds. WT tobacco was included as a control. Table 8 below shows the treatment regimens used.

TABLE 8
treatment regimens for testing BIO3-BIO1 inhibitor resistance

| Treatment | | Application rate | Formulation |
|---|---|---|---|
| no | Compound | (g ai/ha) | and Adjuvant |
| 1 | L | 600 | Genapol X080 |
| | | | 0.2% |
| 2 | A | 600 | Genapol X080 |
| | | | 0.2% |

TABLE 9
Plants transformed with Escherichia coli BioA
with and without mitochondrial signal peptide
(MTP) and BioA from other microbes with MTP

| | | | SEQ | SEQ |
|---|---|---|---|---|
| | | | ID NO of | ID NO |
| | | | Gene back- | with |
| | | | ground | MTP |
| DNA | | | (amino | (amino |
| ID | Name | Gene background | acid) | acid) |
| D4538 | pBin TMV | Escherichia coli | 202 (159) | NA |
| | EcBioA | | | |
| D4539 | pBin TMV | Escherichia coli | 202 (159) | 203 (201) |
| | MTP EcBioA | | | |
| D4555 | pCMP MTP | Escherichia coli | (159) | 203 (201) |
| | EcBioA | | | |
| D4602 | pCMP MTP | Pantoea ananatis | (325) | 333 (325) |
| | PaBioA | | | |
| D4603 | pCMP MTP | Stenotrophomonas | (171) | 334 (326) |
| | SmBioA | maltophilia | | |
| D4609 | pCMP MTP | Chroococcidiopsis | (184) | 335 (327) |
| | ChrBioA | sp. CCMEE 29 | | |
| D4611 | pCMP MTP | Bacillus subtilis | (166) | 336 (328) |
| | BsBioA | | | |
| D4606 | pCMP MTP | Streptomyces | (170) | 337 (329) |
| | SvBioA | viridochromogenes | | |
| D4607 | pCMP MTP | Pedobacter | (181) | 338 (330) |
| | PhBioA | hartonius | | |
| D4608 | pCMP MTP | Chitinophaga | (180) | 339 (331) |
| | CfBioA | filiformis | | |
| D4610 | pCMP MTP | Tenacibaculum | (185) | 340 (332) |
| | TaBioA | adriaticum | | |

The results are shown in Tables 10 to 12 and 16 to 24 below. A control population of wild-type Samsun tobacco was assessed for herbicide damage with result shown in table 9. The wild-type Samsun plants show significant damage at the day 14 and 21 assessment time. The values in Tables 11 and 12 below show the percentage damage scores for populations of 20 plants expressing either the Escherichia coli BioA gene (SEQ ID NO.202) or the Escherichia coli MTP BioA gene (SEQ ID NO.203) at day 7, 14 and 21 post treatment It is clear that the overexpression of Escherichia coli BioA gene with or without MTP (SEQ ID NO.202 and 203) significantly increases tolerance to herbicide with some events showing no damage at all. A photograph was taken of a representative plant from each population (WT Samsun, pBin TMV EcBioA, pBin TMV MTP EcBioA) at 28 days post treatment (FIG. 6). The values in Tables 15-24 show the percentage damage scores for populations of plants expressing bacterial BioAs (SEQ ID Nos: 333-340) under control of the CmYCLV promoter.

TABLE 10
results of percentage of damage to WT tobacco plants

| | Wildtype Samsun tobacco | | | | | |
|---|---|---|---|---|---|---|
| | Compound L (600 gai/ha) | | | Compound A (600 gai/ha) | | |
| WT | 7 DAT | 14 DAT | 21 DAT | 7 DAT | 14 DAT | 21 DAT |
| 1 | 15 | 65 | 85 | 5 | 55 | 50 |
| 2 | 15 | 65 | 85 | 5 | 55 | 45 |
| 3 | 15 | 60 | 80 | 5 | 65 | 70 |
| 4 | 15 | 65 | 85 | 5 | 55 | 65 |
| 5 | 15 | 60 | 85 | 5 | 60 | 75 |
| 6 | 15 | 65 | 80 | 5 | 55 | 45 |
| 7 | 15 | 65 | 90 | 5 | 55 | 50 |
| 8 | 15 | 65 | 85 | 5 | 50 | 45 |

TABLE 11
results of percentage of damage to D4538 (Escherichia coli
BioA) containing tobacco plants

| | pBin TMV EcBioA | | | | | |
|---|---|---|---|---|---|---|
| | Compound L (600 gai/ha) | | | Compound A (600 gai/ha) | | |
| D4538 | 7 DAT | 14 DAT | 21 DAT | 7 DAT | 14 DAT | 21 DAT |
| 8302 | 5 | 0 | 0 | 4 | 15 | 35 |
| 8306 | 5 | 0 | 0 | 4 | 15 | 0 |
| 8307 | 5 | 0 | 0 | 2 | 0 | 0 |
| 8308 | 5 | 0 | 5 | 2 | 10 | 10 |
| 8316 | 5 | 0 | 0 | 2 | 5 | 0 |
| 8323 | 0 | 0 | 0 | 2 | 0 | 5 |
| 8325 | 5 | 0 | 0 | 2 | 0 | 0 |
| 8327 | 2 | 0 | 0 | 0 | 0 | 0 |
| 8328 | 15 | 65 | 85 | 10 | 55 | 70 |
| 8338 | 0 | 0 | 0 | 0 | 0 | 0 |
| 8339 | 5 | 0 | 0 | 2 | 0 | 0 |
| 8341 | 15 | 80 | 90 | 7 | 45 | 60 |
| 8342 | 2 | 0 | 0 | 0 | 0 | 0 |
| 8344 | 2 | 0 | 0 | 0 | 0 | 0 |
| 8345 | 2 | 0 | 0 | 2 | 15 | 20 |
| 8352 | 2 | 0 | 0 | 0 | 3 | 0 |
| 8357 | 2 | 0 | 20 | 2 | 0 | 0 |
| 8363 | 15 | 65 | 85 | 5 | 45 | 50 |
| 8375 | 20 | 70 | 85 | 10 | 55 | 65 |
| 8381 | 10 | 35 | 45 | 7 | 30 | 20 |

TABLE 12
results of percentage of damage to D4539 (Escherichia coli
MTP BioA) containing tobacco plants

| | pBin TMV MTP EcBioA | | | | | |
|---|---|---|---|---|---|---|
| | Compound L (600 gai/ha) | | | Compound A (600 gai/ha) | | |
| D4539 | 7 DAT | 14 DAT | 21 DAT | 7 DAT | 14 DAT | 21 DAT |
| 8385 | 10 | 0 | 0 | 5 | 0 | 0 |
| 8386 | 7 | 0 | 0 | 3 | 0 | 0 |
| 8399 | 2 | 0 | 0 | 2 | 0 | 0 |
| 8402 | 3 | 0 | 0 | 2 | 0 | 0 |
| 8414 | 2 | 0 | 0 | 7 | 30 | 35 |
| 8415 | 7 | 0 | 0 | 7 | 0 | 0 |
| 8417 | 2 | 0 | 20 | 2 | 5 | 10 |
| 8418 | 10 | 25 | 60 | 4 | 10 | 20 |
| 8423 | 2 | 0 | 0 | 2 | 20 | 30 |
| 8424 | 65 | 85 | 90 | 55 | 80 | 85 |
| 8426 | 2 | 0 | 0 | 2 | 0 | 0 |
| 8433 | 5 | 0 | 0 | 4 | 0 | 0 |
| 8434 | 2 | 0 | 35 | 4 | 5 | 15 |
| 8438 | 15 | 85 | 95 | 10 | 75 | 65 |
| 8439 | 2 | 0 | 0 | 2 | 10 | 0 |
| 8447 | 2 | 0 | 0 | 2 | 0 | 0 |
| 8448 | 2 | 0 | 0 | 2 | 0 | 0 |
| 8460 | 2 | 0 | 0 | 3 | 0 | 0 |
| 8461 | 17 | 75 | 90 | 15 | 80 | 85 |
| 8462 | 2 | 0 | 3 | 2 | 0 | 0 |

### Results of Percentage of Damage to:

D4555 (Escherichia coli BioA) containing tobacco plants; 04602 (Pantoea ananatis BioA) containing tobacco plants; 04603 (Stenotrophomonas maltophilia BioA) containing tobacco plants; 04609 (Chroococcidiopsis sp. CCMEE 29 BioA) containing tobacco plants; 04611 (Bacillus subtilis BioA) containing tobacco plants; 04606 (Streptomyces viridochromogenes BioA) containing tobacco plants; 04607 (Pedobacter hartonius BioA) containing tobacco plants; 04608 (Chitinophaga filiformis BioA) containing tobacco plants; and 04610 (Tenacibaculum adriaticum BioA) containing tobacco plants,
   are shown in the “TABLES” section below in Tables 16 to 24 respectively.

### Example 5—Expression and Assay of an Arabidopsis thaliana BIO3-BIO1 (BioDA) Site Saturation Variant Library in E. coli

A pET-24 plasmid containing a DNA sequence encoding a variant of BIO3-BIO1 derived from Arabidopsis thaliana, codon-optimised for expression in E. coli, with the predicted N-terminal mitochondrial transit peptide removed and with an N-terminal 6×His-tag and thrombin cleavage site and under control of a T7 promoter (SEQ ID NO:311), was synthesised by Twist Bioscience. This plasmid was used as the basis of a Site Saturation Variant Library (SSVL) that substituted the codons at 115 sites for codons that encoded one of the other 19 amino acids, or removed the codon altogether, resulting in a translation product with a deletion at that site.

The SSVL was batch-transformed by electroporation into an E. coli BL21(DE3) bioA strain with the bioA gene replaced by CmR conferring resistance to chloramphenicol and spread onto LA supplemented with 50 μg/mL kanamycin and 25 μg/mL chloramphenicol. Each batch consisted of all the plasmids that contained variants at two sites. Colonies from batch transformations were inoculated into 500 μL of M9 minimal media (1×Gibco M9 minimal salts, 2 mM MgSO₄, 0.1 mM CaCl₂, 0.4% (w/v) glucose, 0.4% casamino acids) supplemented with 0.1 mM IPTG, 50 μg/mL kanamycin and 25 μg/mL chloramphenicol. Cultures were grown for three days at 37° C. with shaking whereupon the OD600 was measured of 20 μL subsamples of the cultures diluted in 180 μL of M9 minimal media. The cultures were inoculated to an OD600 of 3×105 into new assay media of 500 μL of M9 minimal media supplemented with 0.1 mM IPTG, 50 μg/mL kanamycin, 25 μg/mL chloramphenicol and 200 ppm of herbicide or DMSO. Assay cultures were grown for two days at 37° C. with shaking with the OD600 being measured at one and two days. Strains that grew in both DMSO and herbicide were determined to be herbicide tolerant. Plasmids were isolated from herbicide-tolerant strains and sequenced to identify the causal mutation in the AtBioDA gene carried by pET-24 in each strain. Identified herbicide tolerant strains were used to express herbicide-tolerant proteins in Example 6. All herbicide-tolerant variants of AtBioDA identified in Example 5 are shown in Table 13.

### Example 6—Expression and Assay of Arabidopsis thaliana BIO3-BIO1 (BioDA) Mutants and Percent-Inhibition Values Versus Various Herbicides

A pet-24 plasmid containing a DNA sequence encoding each identified variant mutant of BIO3-BIO1 derived from Arabidopsis thaliana from example 5 above, as shown in Table 13 below and as shown in full in SEQ ID NOs: 15 to 155, codon-optimised for expression in E. coli, with an N-terminal 6×His-tag and thrombin cleavage site and under control of a T7 promoter, and was synthesised by Twist Bioscience, in a similar manner to SEQ ID NO:311. These plasmids were then expressed in E. coli BL21 (DE3) bioA with 50 μg/mL kanamycin and 25 μg/mL chloramphenicol selection. Overnight cultures grown at 37° C. were used to inoculate 8×500 mL AIM in shake flasks at a ratio of 1:100. Cultures were grown at 37° C., 200 rpm for 3.5 hours and then overnight at 20° C. Cells were harvested by centrifugation for 30 minutes at 10 000 g. Cells were stored at −80° C. until extraction. 30 g of cell pellet from was thawed in 120 mL of IMAC buffer A (20 mM HEPES pH7.5, 25 mM imidazole, 500 mM sodium chloride, 0.15 mM pyridoxal phosphate, 0.5 mM TCEP, 1× complete Protease Inhibitor Tablet (Roche)). The resuspension was passed through a cell disrupter at 18 000 PSI and centrifuged at 50 000 g for 25 minutes at 4° C. The His-tagged BIO3-BIO1 protein was purified from the extract supernatant on an AKTA avant chromatography system (Cytiva) using a HisTrap FF column and eluted in IMAC buffer B ((20 mM HEPES pH7.5, 500 mM imidazole, 500 mM sodium chloride, 0.15 mM pyridoxal phosphate, 0.5 mM TCEP) followed by SEC using a HiLoad 26/60 Superdex200 column in SEC buffer (25 mM HEPES pH7.5, 150 mM sodium chloride, 0.15 mM pyridoxal phosphate, 0.5 mM TCEP). Protein concentration was measured and calculated using the absorbance at 280 nm. Extracts were diluted 1 in 4 and 10 uL was analysed by SDS PAGE.

To measure BIO3-BIO1 mutant enzyme activity in the presence of herbicide, herbicides were incubated with 20 μL of enzyme mix (100 mM EPPS pH 8.6, 0.1 mM pyridoxal phosphate, 0.1 mg/mL BSA, 50 nM BIO3-BIO1 enzyme) for 10 minutes at 20° C. in a well of a 96-well half-area black plate, followed by the addition of 20 μL of substrate mix (final concentration of 100 mM EPPS pH 8.6, 0.1 mM pyridoxal phosphate, 0.1 mg/mL BSA, 100 μM spermidine, 20 μM KAPA, 100 mM NaCO₃) and further incubation for 75 minutes at 20° C. The reaction was terminated by the addition of 50 μL of derivatisation mix (final concentration of 250 mM borate NaOH pH 9.4, 3.6 mM o-phthalaldehyde, 7 mM β-mercaptoethanol, 17% (v/v) ethanol, 3.6% (v/v) methanol). The fluorescent product produced by the reaction of DAPA with o-phthalaldehyde and 7 mM β-mercaptoethanol was measured at 470 nm after excitation at 410 nm. The percentage inhibition was calculated as the activity in the presence of one of three concentrations of each herbicide compound A to M (note that the compounds labelled as A to M are identified hereinabove in the relevant section of the description) as a percentage of the activity in DMSO (Table 12 and 13). The percentage activity versus WT was also calculated and is shown in Table 13 and 14 (NT indicates mutation not tested in table 13, note that these mutations are tested at different compound concentrations for compounds L, M and C, and the results are shown in table 14).

For IC50 calculations, appropriate dose responses of compounds L, M, B, K and G were used. Calculated percentage inhibitions were then input into GraphPad Prism 7 and IC50s calculated using the non-linear regression (vs. normalised response) equation. Results are shown in Table 27.

TABLE 13

| | | Activity | | | | | | | | | | |
|---|---|---|---|---|---|---|---|---|---|---|---|---|
| Variant/Mutant | SEQ | (%) | Compound L | | | Compound M | | | Compound C | | | Compound |
| with respect to | ID | (vs. | 0.01 | 0.075 | 0.5 | 0.2 | 1 | 5 | 0.2 | 1 | 5 | A |
| SEQ ID NO: 1 | NO | WT) | μM | μM | μM | μM | μM | μM | μM | μM | μM | 1 μM |
| WT | 294 | 100 | 6 | 44 | 83 | 23 | 58 | 82 | 29 | 64 | 85 | 59 |
| A507K | 347 | 50 | NT | NT | NT | NT | NT | NT | NT | NT | NT | 81 |
| A507S | 348 | 111 | NT | NT | NT | NT | NT | NT | NT | NT | NT | 63 |
| A609C | 349 | 31 | 4 | 22 | 30 | 10 | 47 | 78 | 29 | 73 | 94 | 18 |
| A609F | 350 | 3 | 10 | 0 | 16 | 19 | 26 | 66 | 5 | 5 | 20 | 6 |
| A609H | 351 | 1 | 0 | 0 | 0 | 7 | 2 | 25 | 0 | 7 | 20 | 0 |
| A609I | 352 | 0 | 29 | 20 | 0 | 2 | 0 | 78 | 7 | 24 | 42 | 24 |
| A609K | 411 | 1 | 36 | 52 | 64 | 0 | 78 | 92 | 65 | 80 | 87 | 18 |
| A609M | 353 | 1 | 3 | 17 | 19 | 5 | 0 | 11 | 9 | 5 | 13 | 3 |
| A609N | 354 | 2 | 83 | 83 | 85 | 44 | 49 | 62 | 45 | 36 | 53 | 78 |
| A609R | 413 | 4 | 0 | 51 | 65 | 52 | 53 | 88 | 63 | 68 | 71 | 54 |
| A609T | 355 | 15 | 10 | 34 | 12 | 8 | 39 | 67 | 12 | 37 | 64 | 0 |
| A609V | 356 | 21 | 0 | 0 | 0 | 22 | 44 | 75 | 0 | 11 | 58 | 5 |
| A609W | 357 | 1 | 35 | 46 | 66 | 24 | 31 | 52 | 21 | 33 | 38 | 34 |
| A609Y | 414 | 8 | 0 | 0 | 0 | 0 | 4 | 45 | 0 | 0 | 43 | 0 |
| C388D | 358 | 3 | 0 | 0 | 6 | 0 | 0 | 0 | 5 | 0 | 9 | 14 |
| C388M | 415 | 101 | 10 | 18 | 34 | 16 | 24 | 41 | 20 | 17 | 69 | 31 |
| C388T | 359 | 175 | 19 | 58 | 92 | 48 | 78 | 95 | 49 | 78 | 95 | 31 |
| F348A | 416 | 31 | 1 | 0 | 3 | 0 | 0 | 6 | 7 | 0 | 1 | 4 |
| F348C | 360 | 15 | 0 | 0 | 29 | 14 | 3 | 0 | 0 | 0 | 0 | 0 |
| F348D | 361 | 1 | 0 | 1 | 2 | 0 | 2 | 0 | 8 | 13 | 0 | 0 |
| F348E | 417 | 2 | 0 | 0 | 32 | 27 | 25 | 66 | 19 | 57 | 86 | 0 |
| F348I | 418 | 36 | 1 | 3 | 14 | 0 | 6 | 25 | 6 | 11 | 49 | 7 |
| F348K | 419 | 19 | 0 | 2 | 22 | 0 | 1 | 24 | 0 | 11 | 15 | 11 |
| F348N | 362 | 13 | 0 | 0 | 21 | 0 | 1 | 15 | 0 | 2 | 17 | 2 |
| F348Q | 421 | 7 | 0 | 0 | 3 | 0 | 0 | 16 | 3 | 0 | 25 | 7 |
| F348S | 363 | 26 | 7 | 3 | 32 | 6 | 4 | 25 | 9 | 7 | 22 | 3 |
| F348T | 364 | 12 | 1 | 3 | 7 | 15 | 2 | 7 | 0 | 4 | 0 | 0 |
| F348V | 365 | 42 | 10 | 15 | 11 | 0 | 0 | 0 | 0 | 0 | 1 | 4 |
| F370L | 422 | 97 | 0 | 14 | 38 | 0 | 25 | 80 | 19 | 24 | 82 | 15 |
| F420I | 423 | 97 | 0 | 1 | 62 | 0 | 45 | 84 | 0 | 55 | 88 | 0 |
| G513A | 424 | 48 | 0 | 57 | 90 | 25 | 58 | 90 | 10 | 63 | 88 | 64 |
| G513DEL | 366 | 49 | 31 | 30 | 54 | 60 | 83 | 95 | 69 | 90 | 98 | 29 |
| G513L | 367 | 14 | 58 | 88 | 97 | 54 | 85 | 97 | 69 | 92 | 98 | 89 |
| G513P | 368 | 61 | 5 | 46 | 86 | 52 | 83 | 97 | 52 | 95 | 99 | 63 |
| G608A | 425 | 93 | 6 | 17 | 68 | 12 | 38 | 74 | 11 | 42 | 81 | 10 |
| G608E | 369 | 1 | 31 | 58 | 68 | 19 | 55 | 85 | 25 | 52 | 81 | 56 |
| G610H | 427 | 111 | 7 | 18 | 65 | 26 | 59 | 84 | 19 | 58 | 82 | 10 |
| G700A | 370 | 10 | 6 | 6 | 3 | 7 | 8 | 6 | 4 | 7 | 28 | 0 |
| G700S | 371 | 10 | 9 | 3 | 3 | 8 | 0 | 9 | 9 | 0 | 9 | 0 |
| L786S | 372 | 98 | 11 | 31 | 93 | 33 | 81 | 97 | 26 | 84 | 98 | 80 |
| M419I | 430 | 102 | 6 | 1 | 30 | 2 | 21 | 20 | 1 | 7 | 53 | 7 |
| P347A | 432 | 101 | 8 | 23 | 47 | 8 | 26 | 52 | 3 | 27 | 51 | 20 |
| P421A | 434 | 112 | 15 | 16 | 21 | 15 | 11 | 17 | 16 | 16 | 35 | 0 |
| P421E | 435 | 0 | 0 | 0 | 41 | 0 | 47 | 62 | 37 | 31 | 0 | 0 |
| P421G | 487 | 110 | 9 | 11 | 85 | 19 | 39 | 75 | 11 | 35 | 80 | 30 |
| P421L | 373 | 34 | 0 | 10 | 45 | 0 | 6 | 31 | 4 | 35 | 67 | 61 |
| P421W | 436 | 93 | 0 | 8 | 27 | 0 | 0 | 18 | 0 | 15 | 37 | 21 |
| P508DEL | 374 | 16 | NT | NT | NT | NT | NT | NT | NT | NT | NT | 22 |
| P508L | 375 | 77 | 16 | 48 | 80 | 18 | 55 | 82 | 37 | 80 | 92 | 34 |
| P508T | 376 | 116 | 25 | 22 | 85 | 20 | 44 | 78 | 23 | 54 | 82 | 55 |
| P510A | 437 | 97 | 0 | 0 | 63 | 0 | 14 | 51 | 0 | 28 | 70 | 4 |
| P510C | 438 | 99 | 0 | 38 | 77 | 9 | 36 | 69 | 13 | 53 | 78 | 20 |
| P510E | 439 | 108 | 0 | 11 | 59 | 0 | 25 | 61 | 8 | 38 | 74 | 26 |
| P510L | 377 | 37 | 35 | 88 | 97 | 54 | 77 | 94 | 67 | 84 | 96 | 82 |
| P510V | 441 | 104 | 0 | 0 | 52 | 0 | 0 | 62 | 0 | 21 | 79 | 1 |
| P529A | 442 | 85 | 0 | 0 | 60 | 0 | 25 | 68 | 4 | 25 | 71 | 0 |
| Q350H | 443 | 104 | 0 | 16 | 38 | 0 | 8 | 20 | 0 | 2 | 25 | 23 |
| Q350S | 444 | 109 | 11 | 38 | 59 | 4 | 22 | 44 | 2 | 35 | 66 | 42 |
| Q506A | 378 | 51 | NT | NT | NT | NT | NT | NT | NT | NT | NT | 0 |
| Q516C | 379 | 31 | 44 | 77 | 95 | 64 | 88 | 97 | 74 | 95 | 99 | 83 |
| Q517D | 380 | 1 | 0 | 0 | 0 | 6 | 2 | 0 | 0 | 0 | 0 | 22 |
| Q517F | 381 | 36 | 0 | 17 | 54 | 0 | 29 | 70 | 29 | 55 | 85 | 20 |
| Q517H | 445 | 55 | 0 | 4 | 51 | 6 | 37 | 71 | 11 | 44 | 77 | 0 |
| Q517I | 446 | 62 | 14 | 35 | 65 | 19 | 44 | 72 | 25 | 58 | 82 | 28 |
| Q517M | 382 | 83 | NT | NT | NT | NT | NT | NT | NT | NT | NT | 38 |
| Q517T | 383 | 35 | 9 | 50 | 76 | 14 | 39 | 71 | 14 | 47 | 73 | 46 |
| Q517W | 447 | 50 | 14 | 32 | 65 | 26 | 51 | 79 | 29 | 72 | 90 | 9 |
| Q517Y | 384 | 78 | 8 | 38 | 74 | 36 | 71 | 90 | 49 | 86 | 97 | 52 |
| R756K | 448 | 101 | 6 | 30 | 64 | 10 | 36 | 71 | 6 | 42 | 80 | 33 |
| R756S | 385 | 106 | 10 | 49 | 82 | 23 | 54 | 78 | 24 | 57 | 83 | 67 |
| R790C | 449 | 88 | 0 | 4 | 55 | 0 | 9 | 49 | 0 | 11 | 62 | 2 |
| R797Q | 450 | 4 | 5 | 10 | 26 | 0 | 5 | 30 | 7 | 32 | 52 | 35 |
| S390C | 451 | 104 | 1 | 45 | 82 | 16 | 44 | 74 | 27 | 55 | 83 | 21 |
| S509A | 453 | 97 | 0 | 0 | 20 | 0 | 33 | 81 | 0 | 63 | 95 | 0 |
| S509C | 386 | 14 | 12 | 41 | 62 | 21 | 53 | 78 | 30 | 65 | 88 | 0 |
| S509D | 387 | 18 | 14 | 14 | 63 | 48 | 80 | 98 | 59 | 91 | 103 | 9 |
| S509E | 388 | 28 | 0 | 22 | 41 | 40 | 73 | 93 | 57 | 87 | 97 | 6 |
| S509F | 454 | 35 | 3 | 0 | 18 | 19 | 58 | 89 | 24 | 82 | 98 | 6 |
| S509G | 389 | 19 | 30 | 69 | 84 | 28 | 47 | 71 | 20 | 53 | 75 | 36 |
| S509H | 455 | 52 | 0 | 4 | 31 | 23 | 82 | 98 | 20 | 75 | 98 | 0 |
| S509I | 456 | 20 | 3 | 2 | 22 | 30 | 65 | 92 | 38 | 84 | 99 | 0 |
| S509K | 457 | 17 | 0 | 26 | 42 | 41 | 77 | 90 | 54 | 88 | 95 | 0 |
| S509L | 458 | 62 | 0 | 3 | 31 | 19 | 68 | 94 | 24 | 85 | 99 | 6 |
| S509N | 460 | 102 | 3 | 5 | 65 | 6 | 39 | 78 | 16 | 50 | 86 | 43 |
| S509Q | 461 | 79 | 0 | 11 | 41 | 23 | 76 | 96 | 23 | 82 | 99 | 8 |
| S509R | 462 | 22 | 0 | 20 | 61 | 48 | 82 | 96 | 42 | 87 | 98 | 15 |
| S509T | 463 | 103 | 3 | 58 | 91 | 9 | 69 | 95 | 5 | 50 | 89 | 77 |
| S509V | 390 | 117 | 12 | 10 | 11 | 22 | 26 | 88 | 17 | 30 | 94 | 6 |
| S509W | 391 | 24 | 0 | 0 | 0 | 0 | 60 | 95 | 14 | 92 | 103 | 0 |
| S509Y | 464 | 35 | 3 | 0 | 19 | 28 | 58 | 85 | 37 | 76 | 95 | 3 |
| S704H | 392 | 1 | 0 | 0 | 0 | 0 | 0 | 0 | 0 | 0 | 0 | 0 |
| S704P | 465 | 2 | 0 | 0 | 42 | 0 | 16 | 47 | 1 | 23 | 60 | 56 |
| T393V | 466 | 97 | 9 | 19 | 65 | 0 | 19 | 46 | 3 | 31 | 70 | 10 |
| T512C | 393 | 110 | 0 | 18 | 69 | 27 | 79 | 95 | 22 | 83 | 97 | 50 |
| T512D | 394 | 6 | 24 | 23 | 58 | 59 | 86 | 96 | 79 | 95 | 97 | 33 |
| T512G | 467 | 96 | 0 | 0 | 20 | 9 | 39 | 77 | 17 | 57 | 92 | 5 |
| T512I | 468 | 43 | 2 | 15 | 29 | 48 | 85 | 96 | 60 | 96 | 99 | 37 |
| T512N | 469 | 67 | 15 | 37 | 76 | 27 | 74 | 93 | 29 | 74 | 93 | 21 |
| T512Q | 395 | 78 | 3 | 35 | 74 | 49 | 91 | 99 | 53 | 96 | 100 | 49 |
| T512R | 396 | 15 | 27 | 61 | 91 | 81 | 95 | 99 | 74 | 95 | 99 | 82 |
| T512V | 397 | 48 | 33 | 57 | 83 | 57 | 77 | 94 | 51 | 81 | 96 | 58 |
| T512W | 470 | 11 | 18 | 0 | 31 | 33 | 55 | 83 | 50 | 83 | 95 | 0 |
| V354A | 471 | 100 | 6 | 0 | 47 | 6 | 13 | 65 | 0 | 26 | 75 | 9 |
| V354E | 472 | 108 | 4 | 19 | 35 | 0 | 0 | 23 | 0 | 0 | 39 | 25 |
| V354L | 473 | 86 | 0 | 16 | 63 | 4 | 43 | 78 | 0 | 32 | 86 | 1 |
| V354T | 475 | 97 | 0 | 7 | 89 | 4 | 47 | 88 | 0 | 56 | 92 | 61 |
| W391L | 398 | 5 | 24 | 10 | 16 | 18 | 16 | 43 | 23 | 29 | 33 | 62 |
| W391M | 399 | 1 | 0 | 16 | 30 | 27 | 37 | 84 | 39 | 24 | 63 | 85 |
| W392A | 400 | 3 | NT | NT | NT | NT | NT | NT | NT | NT | NT | 53 |
| W392C | 477 | 28 | 2 | 12 | 13 | 0 | 17 | 27 | 20 | 55 | 87 | 42 |
| W392D | 478 | 2 | 19 | 59 | 85 | 14 | 37 | 81 | 46 | 59 | 91 | 55 |
| W392G | 401 | 9 | 0 | 2 | 0 | 0 | 7 | 12 | 0 | 20 | 69 | 56 |
| W392M | 479 | 19 | 0 | 10 | 8 | 0 | 0 | 22 | 10 | 30 | 56 | 14 |
| W392S | 402 | 10 | 0 | 3 | 0 | 0 | 0 | 0 | 0 | 0 | 20 | 23 |
| Y511C | 480 | 33 | 0 | 3 | 44 | 16 | 67 | 92 | 33 | 86 | 98 | 0 |
| Y511D | 403 | 6 | 20 | 29 | 51 | 38 | 81 | 96 | 68 | 93 | 99 | 10 |
| Y511E | 404 | 13 | 0 | 0 | 33 | 29 | 74 | 95 | 27 | 74 | 96 | 0 |
| Y511F | 405 | 17 | 48 | 65 | 86 | 24 | 57 | 82 | 44 | 67 | 90 | 79 |
| Y511I | 482 | 60 | 21 | 27 | 28 | 21 | 81 | 96 | 36 | 93 | 100 | 17 |
| Y511M | 406 | 54 | 0 | 27 | 76 | 41 | 83 | 97 | 76 | 99 | 100 | 59 |
| Y511P | 407 | 13 | 10 | 26 | 63 | 48 | 89 | 98 | 53 | 95 | 99 | 28 |
| Y511Q | 484 | 8 | 0 | 6 | 28 | 54 | 87 | 95 | 78 | 97 | 98 | 21 |
| Y511R | 408 | 8 | 15 | 7 | 34 | 74 | 96 | 99 | 80 | 99 | 99 | 46 |
| Y511V | 409 | 58 | 0 | 5 | 49 | 33 | 88 | 99 | 28 | 94 | 97 | 45 |
| Y511W | 410 | 51 | 11 | 22 | 50 | 25 | 67 | 90 | 41 | 85 | 97 | 57 |

| Variant/Mutant | Compound A | | Compound D | | | Compound B | | | Compound E | | |
|---|---|---|---|---|---|---|---|---|---|---|---|
| with respect to | 5 | 25 | 0.01 | 0.075 | 0.5 | 20 | 100 | 200 | 20 | 100 | 200 |
| SEQ ID NO: 1 | μM | μM | μM | μM | μM | μM | μM | μM | μM | μM | μM |
| WT | 84 | 96 | 51 | 84 | 95 | 84 | 95 | 96 | 80 | 94 | 96 |
| A507K | 97 | 100 | 37 | 97 | 100 | 67 | 91 | 95 | 66 | 92 | 96 |
| A507S | 87 | 97 | 24 | 83 | 96 | 76 | 94 | 96 | 74 | 93 | 96 |
| A609C | 50 | 79 | 43 | 82 | 96 | 58 | 86 | 92 | 23 | 56 | 73 |
| A609F | 13 | 18 | 0 | 0 | 7 | 30 | 57 | 72 | 8 | 24 | 39 |
| A609H | 0 | 0 | 0 | 12 | 22 | 0 | 24 | 14 | 0 | 1 | 0 |
| A609I | 11 | 96 | 33 | 0 | 24 | 60 | 33 | 0 | 64 | 56 | 56 |
| A609K | 58 | 74 | 62 | 72 | 78 | 76 | 75 | 75 | 75 | 66 | 65 |
| A609M | 0 | 0 | 0 | 0 | 0 | 18 | 2 | 0 | 0 | 0 | 0 |
| A609N | 86 | 90 | 41 | 42 | 42 | 39 | 46 | 49 | 34 | 27 | 29 |
| A609R | 59 | 69 | 73 | 73 | 73 | 73 | 83 | 85 | 71 | 71 | 70 |
| A609T | 25 | 54 | 7 | 33 | 63 | 54 | 82 | 88 | 17 | 30 | 46 |
| A609V | 24 | 64 | 0 | 12 | 64 | 33 | 65 | 82 | 35 | 71 | 82 |
| A609W | 66 | 84 | 39 | 52 | 61 | 51 | 48 | 35 | 52 | 46 | 52 |
| A609Y | 0 | 2 | 2 | 13 | 48 | 15 | 44 | 59 | 8 | 0 | 10 |
| C388D | 25 | 62 | 1 | 8 | 5 | 0 | 13 | 20 | 0 | 0 | 0 |
| C388M | 66 | 92 | 21 | 57 | 87 | 66 | 89 | 94 | 44 | 73 | 86 |
| C388T | 81 | 97 | 22 | 81 | 97 | 40 | 81 | 94 | 54 | 85 | 90 |
| F348A | 6 | 2 | 2 | 0 | 12 | 96 | 99 | 99 | 2 | 15 | 25 |
| F348C | 0 | 0 | 2 | 2 | 10 | 94 | 98 | 98 | 8 | 21 | 26 |
| F348D | 0 | 2 | 3 | 0 | 25 | 31 | 46 | 57 | 0 | 0 | 0 |
| F348E | 33 | 56 | 37 | 64 | 76 | 77 | 90 | 91 | 61 | 79 | 76 |
| F348I | 14 | 28 | 18 | 47 | 89 | 97 | 99 | 100 | 48 | 82 | 90 |
| F348K | 17 | 14 | 17 | 14 | 21 | 69 | 90 | 94 | 25 | 59 | 68 |
| F348N | 0 | 4 | 15 | 12 | 28 | 76 | 89 | 91 | 0 | 0 | 17 |
| F348Q | 3 | 3 | 0 | 17 | 17 | 51 | 82 | 89 | 13 | 16 | 0 |
| F348S | 19 | 8 | 2 | 29 | 20 | 85 | 95 | 96 | 9 | 18 | 16 |
| F348T | 1 | 4 | 2 | 11 | 28 | 92 | 97 | 98 | 4 | 23 | 40 |
| F348V | 12 | 39 | 0 | 13 | 48 | 88 | 95 | 96 | 2 | 18 | 31 |
| F370L | 17 | 72 | 10 | 61 | 95 | 45 | 83 | 90 | 39 | 72 | 84 |
| F420I | 14 | 53 | 15 | 73 | 95 | 42 | 86 | 91 | 40 | 85 | 90 |
| G513A | 92 | 98 | 56 | 87 | 97 | 61 | 85 | 90 | 70 | 89 | 94 |
| G513DEL | 70 | 91 | 73 | 98 | 98 | 11 | 97 | 98 | 81 | 96 | 98 |
| G513L | 98 | 97 | 93 | 99 | 100 | 80 | 95 | 98 | 82 | 96 | 98 |
| G513P | 91 | 99 | 60 | 98 | 100 | 24 | 94 | 97 | 76 | 94 | 97 |
| G608A | 49 | 87 | 17 | 74 | 94 | 71 | 91 | 95 | 40 | 81 | 89 |
| G608E | 74 | 88 | 41 | 69 | 87 | 66 | 85 | 86 | 42 | 65 | 70 |
| G610H | 28 | 70 | 27 | 64 | 87 | 80 | 95 | 97 | 29 | 66 | 81 |
| G700A | 0 | 21 | 4 | 37 | 73 | 76 | 95 | 97 | 6 | 27 | 36 |
| G700S | 0 | 0 | 8 | 8 | 42 | 28 | 71 | 83 | 0 | 0 | 0 |
| L786S | 96 | 98 | 49 | 97 | 101 | 69 | 93 | 98 | 57 | 90 | 94 |
| M419I | 20 | 62 | 15 | 45 | 73 | 75 | 93 | 95 | 69 | 90 | 93 |
| P347A | 35 | 64 | 43 | 64 | 83 | 52 | 83 | 90 | 26 | 62 | 74 |
| P421A | 5 | 51 | 0 | 9 | 69 | 44 | 83 | 91 | 7 | 75 | 81 |
| P421E | 0 | 0 | 0 | 39 | 62 | 0 | 23 | 0 | 0 | 0 | 0 |
| P421G | 59 | 32 | 48 | 77 | 89 | 75 | 93 | 94 | 61 | 83 | 80 |
| P421L | 87 | 102 | 1 | 18 | 55 | 52 | 78 | 90 | 2 | 6 | 15 |
| P421W | 28 | 72 | 23 | 55 | 76 | 77 | 93 | 96 | 72 | 92 | 95 |
| P508DEL | 56 | 83 | 62 | 95 | 100 | 79 | 96 | 98 | 86 | 98 | 99 |
| P508L | 75 | 89 | 43 | 90 | 97 | 82 | 95 | 96 | 77 | 93 | 96 |
| P508T | 84 | 94 | 38 | 86 | 95 | 14 | 87 | 97 | 0 | 83 | 96 |
| P510A | 43 | 76 | 26 | 67 | 91 | 67 | 92 | 95 | 50 | 87 | 93 |
| P510C | 60 | 86 | 37 | 79 | 94 | 66 | 90 | 95 | 55 | 86 | 92 |
| P510E | 38 | 79 | 4 | 66 | 91 | 55 | 88 | 92 | 60 | 91 | 95 |
| P510L | 97 | 99 | 85 | 97 | 99 | 86 | 96 | 98 | 81 | 95 | 97 |
| P510V | 48 | 85 | 12 | 67 | 92 | 54 | 87 | 92 | 44 | 79 | 86 |
| P529A | 26 | 73 | 0 | 65 | 92 | 66 | 91 | 95 | 46 | 88 | 92 |
| Q350H | 49 | 79 | 7 | 34 | 69 | 100 | 86 | 92 | 47 | 81 | 89 |
| Q350S | 61 | 83 | 100 | 65 | 87 | 73 | 91 | 94 | 64 | 86 | 91 |
| Q506A | 25 | 63 | 21 | 56 | 86 | 83 | 94 | 96 | 85 | 94 | 96 |
| Q516C | 96 | 99 | 95 | 99 | 100 | 83 | 96 | 97 | 84 | 97 | 98 |
| Q517D | 54 | 70 | 0 | 0 | 5 | 26 | 46 | 52 | 0 | 15 | 22 |
| Q517F | 51 | 84 | 42 | 76 | 95 | 85 | 96 | 98 | 83 | 96 | 98 |
| Q517H | 32 | 66 | 20 | 61 | 89 | 79 | 94 | 97 | 75 | 92 | 96 |
| Q517I | 53 | 80 | 55 | 82 | 94 | 80 | 94 | 97 | 75 | 93 | 96 |
| Q517M | 55 | 92 | 47 | 83 | 95 | 89 | 97 | 98 | 88 | 96 | 98 |
| Q517T | 72 | 90 | 38 | 73 | 87 | 89 | 97 | 98 | 82 | 95 | 78 |
| Q517W | 42 | 59 | 51 | 84 | 96 | 64 | 89 | 94 | 59 | 87 | 93 |
| Q517Y | 79 | 95 | 70 | 95 | 99 | 87 | 97 | 98 | 83 | 96 | 98 |
| R756K | 70 | 92 | 71 | 90 | 95 | 57 | 86 | 92 | 33 | 62 | 89 |
| R756S | 88 | 97 | 47 | 81 | 94 | 83 | 95 | 97 | 76 | 93 | 96 |
| R790C | 37 | 68 | 0 | 70 | 90 | 58 | 92 | 94 | 41 | 87 | 91 |
| R797Q | 28 | 64 | 39 | 38 | 50 | 43 | 50 | 52 | 44 | 48 | 43 |
| S390C | 66 | 88 | 54 | 84 | 95 | 68 | 91 | 94 | 53 | 87 | 92 |
| S509A | 0 | 7 | 4 | 83 | 98 | 35 | 80 | 89 | 50 | 88 | 93 |
| S509C | 26 | 52 | 61 | 84 | 94 | 88 | 97 | 98 | 89 | 98 | 99 |
| S509D | 22 | 57 | 72 | 97 | 103 | 80 | 99 | 101 | 87 | 101 | 102 |
| S509E | 24 | 63 | 73 | 95 | 99 | 84 | 96 | 97 | 92 | 98 | 99 |
| S509F | 15 | 37 | 51 | 93 | 99 | 60 | 87 | 93 | 75 | 94 | 95 |
| S509G | 71 | 87 | 60 | 82 | 93 | 5 | 88 | 97 | 14 | 89 | 97 |
| S509H | 25 | 78 | 19 | 89 | 100 | 33 | 71 | 85 | 41 | 78 | 88 |
| S509I | 1 | 37 | 51 | 93 | 99 | 41 | 78 | 90 | 54 | 85 | 91 |
| S509K | 4 | 35 | 70 | 91 | 96 | 42 | 71 | 81 | 43 | 79 | 85 |
| S509L | 8 | 31 | 38 | 92 | 99 | 58 | 85 | 92 | 68 | 88 | 93 |
| S509N | 57 | 74 | 59 | 80 | 92 | 70 | 88 | 90 | 57 | 77 | 80 |
| S509Q | 18 | 52 | 31 | 93 | 100 | 52 | 82 | 88 | 60 | 89 | 93 |
| S509R | 39 | 68 | 52 | 0 | 99 | 45 | 80 | 83 | 55 | 84 | 89 |
| S509T | 92 | 98 | 79 | 93 | 93 | 70 | 91 | 92 | 36 | 33 | 17 |
| S509V | 12 | 49 | 0 | 54 | 98 | 0 | 64 | 91 | 4 | 73 | 94 |
| S509W | 0 | 9 | 56 | 102 | 104 | 57 | 93 | 98 | 71 | 98 | 101 |
| S509Y | 4 | 23 | 43 | 84 | 97 | 63 | 88 | 93 | 66 | 88 | 94 |
| S704H | 100 | 100 | 0 | 0 | 96 | 0 | 0 | 72 | 0 | 0 | 0 |
| S704P | 56 | 49 | 66 | 79 | 78 | 90 | 104 | 99 | 69 | 76 | 72 |
| T393V | 34 | 67 | 23 | 67 | 90 | 72 | 93 | 97 | 52 | 83 | 92 |
| T512C | 81 | 94 | 44 | 93 | 99 | 82 | 94 | 97 | 88 | 96 | 98 |
| T512D | 51 | 87 | 93 | 97 | 98 | 41 | 79 | 86 | 67 | 88 | 93 |
| T512G | 6 | 46 | 21 | 72 | 96 | 76 | 94 | 97 | 78 | 95 | 97 |
| T512I | 50 | 65 | 90 | 98 | 99 | 59 | 77 | 84 | 75 | 89 | 93 |
| T512N | 61 | 88 | 50 | 85 | 97 | 80 | 95 | 97 | 79 | 95 | 97 |
| T512Q | 87 | 97 | 63 | 99 | 100 | 78 | 96 | 97 | 87 | 98 | 98 |
| T512R | 96 | 99 | 85 | 96 | 100 | 57 | 80 | 88 | 73 | 90 | 95 |
| T512V | 81 | 94 | 63 | 91 | 97 | 86 | 96 | 98 | 87 | 97 | 98 |
| T512W | 3 | 19 | 69 | 93 | 96 | 47 | 76 | 87 | 77 | 91 | 93 |
| V354A | 25 | 79 | 1 | 57 | 92 | 56 | 87 | 93 | 43 | 85 | 90 |
| V354E | 52 | 78 | 5 | 18 | 80 | 76 | 93 | 96 | 63 | 86 | 92 |
| V354L | 48 | 86 | 0 | 67 | 94 | 62 | 89 | 94 | 60 | 90 | 92 |
| V354T | 79 | 91 | 77 | 91 | 97 | 85 | 97 | 97 | 84 | 97 | 97 |
| W391L | 100 | 137 | 20 | 36 | 55 | 74 | 82 | 96 | 38 | 21 | 35 |
| W391M | 94 | 108 | 28 | 48 | 104 | 77 | 92 | 90 | 15 | 17 | 15 |
| W392A | 81 | 97 | 47 | 97 | 101 | 31 | 52 | 70 | 24 | 68 | 88 |
| W392C | 69 | 90 | 69 | 92 | 97 | 39 | 57 | 62 | 43 | 72 | 72 |
| W392D | 73 | 93 | 83 | 90 | 97 | 79 | 89 | 85 | 80 | 88 | 87 |
| W392G | 66 | 110 | 55 | 93 | 100 | 29 | 45 | 72 | 41 | 73 | 87 |
| W392M | 37 | 79 | 22 | 85 | 98 | 9 | 46 | 58 | 37 | 73 | 80 |
| W392S | 68 | 93 | 14 | 75 | 95 | 6 | 33 | 54 | 43 | 80 | 89 |
| Y511C | 19 | 50 | 48 | 94 | 99 | 44 | 74 | 86 | 59 | 89 | 93 |
| Y511D | 35 | 72 | 77 | 96 | 100 | 38 | 70 | 81 | 60 | 85 | 93 |
| Y511E | 16 | 62 | 39 | 90 | 98 | 39 | 75 | 84 | 64 | 88 | 92 |
| Y511F | 86 | 97 | 88 | 92 | 98 | 83 | 95 | 96 | 90 | 98 | 98 |
| Y511I | 28 | 65 | 33 | 98 | 100 | 44 | 73 | 87 | 76 | 94 | 97 |
| Y511M | 84 | 97 | 94 | 100 | 100 | 83 | 95 | 97 | 94 | 99 | 99 |
| Y511P | 57 | 88 | 82 | 98 | 99 | 42 | 81 | 87 | 63 | 91 | 94 |
| Y511Q | 32 | 74 | 87 | 99 | 98 | 32 | 64 | 77 | 63 | 83 | 89 |
| Y511R | 85 | 97 | 94 | 99 | 99 | 39 | 81 | 84 | 79 | 95 | 95 |
| Y511V | 81 | 96 | 54 | 99 | 100 | 59 | 82 | 91 | 85 | 96 | 98 |
| Y511W | 87 | 97 | 80 | 98 | 100 | 90 | 97 | 99 | 99 | 100 | 100 |

TABLE 14

| | | Activity | | | | | | | | | |
|---|---|---|---|---|---|---|---|---|---|---|---|
| Variant/Mutant | SEQ | (%) | Compound L | | | Compound M | | | Compound C | | |
| with respect to | ID | (vs. | 0.1 | 10 | 100 | 0.1 | 10 | 100 | 0.1 | 10 | 100 |
| SEQ ID NO: 1 | NO | WT) | μM | μM | μM | μM | μM | μM | μM | μM | μM |
| WT | 294 | 100 | 70 | 99 | 99 | 23 | 92 | 99 | 27 | 92 | 99 |
| C388T | 359 | 175 | 53 | 99 | 100 | 33 | 98 | 100 | 23 | 98 | 100 |
| C388D | 358 | 3 | 7 | 21 | 41 | 11 | 12 | 20 | 8 | 15 | 19 |
| P421L | 373 | 34 | 14 | 88 | 99 | 3 | 53 | 93 | 4 | 80 | 97 |
| W391L | 398 | 5 | 17 | 49 | 96 | 17 | 73 | 100 | 16 | 31 | 70 |
| W392G | 401 | 9 | 9 | 51 | 86 | 6 | 27 | 70 | 6 | 76 | 98 |
| W391M | 399 | 1 | 27 | 99 | 93 | 28 | 82 | 87 | 26 | 82 | 87 |
| W392A | 400 | 3 | 11 | 48 | 86 | 0 | 0 | 55 | 1 | 67 | 96 |
| Q506A | 378 | 51 | 0 | 81 | 96 | 0 | 83 | 98 | 5 | 89 | 98 |
| P508L | 375 | 77 | 10 | 92 | 96 | 1 | 85 | 97 | 5 | 94 | 98 |
| A507K | 347 | 50 | 23 | 100 | 100 | 14 | 97 | 100 | 1 | 98 | 100 |
| A507S | 348 | 111 | 32 | 98 | 100 | 22 | 84 | 98 | 20 | 90 | 98 |
| P508del | 374 | 16 | 11 | 75 | 98 | 2 | 97 | 99 | 15 | 99 | 99 |
| Q517M | 382 | 83 | 31 | 97 | 99 | 34 | 88 | 98 | 34 | 93 | 99 |
| Q517Y | 384 | 78 | 14 | 97 | 99 | 1 | 89 | 99 | 0 | 98 | 100 |
| Q517F | 381 | 36 | 20 | 93 | 99 | 18 | 84 | 97 | 14 | 93 | 99 |
| G700A | 370 | 10 | 0 | 31 | 80 | 0 | 3 | 59 | 0 | 58 | 94 |

The activity of various wild-type BioDA and variant enzymes was also tested when treated with DMSO or herbicide in vitro.

Protein of Schizosaccharomyces japonicus BioDA (SEQ ID NO:13), Nannochloropsis gaditana BioDA (SEQ ID NO:3), Selaginella moellendorffii BioDA (SEQ ID NO:319), Quercus robur BioDA (SEQ ID NOA11), Ostreococcus tauri BioDA (SEQ ID NO:9), and the variants of Ostreococcus tauri BioDA A374D, F332A and P407A (SEQ ID Nos:321-323) was prepared using the method in Example 6. BioDA enzyme activity in the presence of herbicide or DMSO in vitro for each of the proteins was assayed according to the enzyme assay method above. The results are shown in Table 26 provided below in the “TABLES” section.

In addition, Table 27 shows the IC50 of Arabidopsis thaliana BioDA variant enzymes in relation to Compound L, Compound M, Compound C, Compound B, Compound K and Compound G.

### Example 7—Assay of Herbicide Tolerance in Transgenic Tobacco Plants Expressing Heterologous Arabidopsis thaliana or Zea mays BIO3-BIO1 (BioDA) Mutants

To determine if overexpression of variants detailed in Example 7 confer resistance to BIO3-BIO1 inhibiting compounds when expressed in plants, GM tobacco lines were produced using the methods in Example 1 and tested with BIO3-BIO1 inhibiting compounds.

Transgenic tobacco plants expressing the Arabidopsis thaliana BIO3-BIO1 variants C388D (SEQ ID NO:26), P421L(SEQ ID NO:41), F348C (SEQ ID NO:28), G700S (SEQ ID NO:39), W392S (SEQ ID NO:70) and the Zea mays BIO3-BIO1 variant C372D (SEQ ID NO:324). Populations of transgenic tobacco were sprayed and assessed as in Example 2

The plants and DNA IDs of each transgenic plant are shown in Table 28 provided in the “TABLES” section below.

Results of percentage damage for each transgenic plant are shown in Tables 29 to 35 in the “TABLES” section below.

### Example 8: Expression and Assay of Wild-Type BioA Proteins

Protein of Escherichia coli BioA (SEQ ID NO:159), Pantoea ananatis BioA (SEQ ID NO:167), Streptomyces viridochromogenes BioA (SEQ ID NO:170), Cryptosporidium andersoni BioA (SEQ ID NO:160), Stenotrophomonas maltophilia BioA (SEQ ID NO:171), Chroococcidiopsis BioA (SEQ ID NO:184), Pedobacter hartonius BioA (SEQ ID NO:181), Chitinophaga filiformis BioA (SEQ ID NO:180), Tenacibaculum adriaticum BioA (SEQ ID NO:185) and Bacillus subtilis BioA (SEQ ID NO:166) was prepared using the method in Example 6. BioA enzyme activity in the presence of herbicide or DMSO in vitro for each of the proteins was assayed according to the enzyme assay method in Example 6, except in some reactions spermidine was replaced with 2 mM S-Adenosyl methionine.

### Example 9: Genome Editing in Crops

Maize, soy, rice or sunflower genomic DNA encoding a BIO3-BIO1 enzyme is edited to produce a corn, soy, rice or sunflower plant having tolerance to BIO3-BIO1-inhibiting herbicides. A vector construct is created targeting maize (e.g. the vector of FIG. 7 (SEQ ID NO: 204) or vectors targeting soy (SEQ ID NO: 312), rice (SEQ ID NO: 313) or sunflower (SEQ ID NO: 314)) using CpF1 and at least one gRNA to mediate one or more of the following amino acid substitution mutations to the native BIO3-BIO1 enzyme: C372D, C372T, A495S, F333C, F333N, F333S, F333T, F333V, G687A, G687S, P405L, P496T, R743S, S497V, S497W, W375G, and/or W376S. Example donor sequences are shown in SEQ ID NO: 205 (maize), SEQ ID NO: 315 (soy—derived from Glycine max Williams 82), SEQ ID NO: 316 (rice—derived from Oryza sativa Nipponbare) and SEQ ID NO: 317 (sunflower—Helianthus annuus XRQ). Maize, soy, rice or sunflower are transformed with the vector using a technique well known in the art, for example particle bombardment or Agrobacterium-mediated transformation. For corn, see for example, Svitashev, S., Schwartz, C., Lenderts, B. et al. Genome editing in maize directed by CRISPR-Cas9 ribonucleoprotein complexes. Nat Commun 7, 13274 (2016); Front. Plant Sci., 5 Aug. 2014, Sec. Plant Biotechnology Volume 5-2014, herein incorporated by reference. For soybean, see, for example, Liu, J., Gunapati, S., Mihelich, N.T., Stec, A. O., Michno, J M., Stupar, R. M. (2019). Genome Editing in Soybean with CRISPR/Cas9. In: Qi, Y. (eds) Plant Genome Editing with CRISPR Systems. Methods in Molecular Biology, vol 1917. Humana, New York, NY.; Liang, D. et al. (2023). CRISPR/LbCasl2a-Mediated Genome Editing in Soybean. In: Yang, B., Harwood, W., Que, Q. (eds) Plant Genome Engineering. Methods in Molecular Biology, vol 2653. Humana, New York, NY; Pareddy, D., Chennareddy, S., Anthony, G. et al. Improved soybean transformation for efficient and high throughput transgenic production. Transgenic Res 29, 267-281 (2020)., each of which is herein incorporated by reference. For rice, see for example, Yu, K., Liu, Z., Gui, H. et al. Highly efficient generation of bacterial leaf blight-resistant and transgene-free rice using a genome editing and multiplexed selection system. BMC Plant Biol 21, 197 (2021), Christou, P. (1997). Rice transformation: bombardment. In: Sasaki, T., Moore, G. (eds) Oryza: From Molecule to Plant. Springer, Dordrecht, and Nishimura, A., Aichi, I. & Matsuoka, M. A protocol for Agrobacterium-mediated transformation in rice. Nat Protoc 1, 2796-2802 (2006), each of which is herein incorporated by reference. For sunflower, see for example, Knittel, N., Gruber, V., Hahne, G. et al. Transformation of sunflower (Helianthus annuus L.): a reliable protocol. Plant Cell Reports 14, 81-86 (1994, herein incorporated by reference. Putative edited plants are screened with BIO3-BIO1 herbicide inhibitors, such as those described above, to evaluate resistance relative to a non-edited control plant.

TABLES
Table 15: results of percentage of damage to D4612
(Ostreococcus tauri BIO3-BIO1) containing tobacco plants

| | pCMP OtBioDA | | | | | |
|---|---|---|---|---|---|---|
| | Compound L (600 gai/ha) | | | Compound A (600 gai/ha) | | |
| D4581 | 7 DAT | 14 DAT | 21 DAT | 7 DAT | 14 DAT | 21 DAT |
| 1744 | 0 | 0 | 0 | 0 | 0 | 0 |
| 1752 | 40 | 85 | 90 | 8 | 35 | 40 |
| 1754 | 20 | 15 | 5 | 0 | 0 | 0 |
| 1761 | 0 | 0 | 0 | 0 | 0 | 0 |
| 1763 | 0 | 0 | 0 | 0 | 0 | 0 |
| 1764 | 20 | 5 | 0 | 0 | 0 | 0 |
| 1766 | 25 | 10 | 5 | 0 | 0 | 0 |
| 1767 | 0 | 0 | 0 | 0 | 0 | 0 |
| 1770 | 10 | 5 | 2 | 0 | 0 | 0 |
| 1779 | 15 | 5 | 3 | 0 | 0 | 0 |
| 1781 | 0 | 0 | 0 | 0 | 0 | 0 |
| 1789 | 5 | 5 | 0 | 0 | 0 | 0 |
| 1791 | 40 | 75 | 90 | 10 | 60 | 70 |
| 1795 | 0 | 0 | 0 | 0 | 0 | 0 |
| 1796 | 0 | 0 | 0 | 0 | 0 | 0 |
| 1800 | 0 | 0 | 0 | 0 | 0 | 0 |
| 1801 | 0 | 0 | 0 | 0 | 0 | 0 |
| 1805 | 45 | 80 | 90 | 10 | 50 | 35 |
| 1809 | 0 | 0 | 0 | 0 | 0 | 0 |
| 1820 | 40 | 85 | 85 | 10 | 40 | 30 |

TABLE 16
results of percentage of damage to D4555 (Escherichia coli
BioA) containing tobacco plants. Three replicate clones (A, B,
C) for two events (8480, 8526) are shown for each treatment.

| | pCMP MTP EcBioA | | | | | |
|---|---|---|---|---|---|---|
| | Compound L (600 gai/ha) | | | Compound A (600 gai/ha) | | |
| D4555 | 7 DAT | 14 DAT | 21 DAT | 7 DAT | 14 DAT | 21 DAT |
| 8480 | | | | | | |
| A | 0 | 0 | 0 | 0 | 0 | 0 |
| B | 0 | 0 | 0 | 0 | 0 | 0 |
| C | 0 | 0 | 0 | 0 | 0 | 0 |
| 8526 | | | | | | |
| A | 0 | 0 | 0 | 0 | 0 | 0 |
| B | 0 | 0 | 0 | 0 | 0 | 0 |
| C | 0 | 0 | 0 | 0 | 0 | 0 |

TABLE 17
results of percentage of damage to D4602 (Pantoea ananatis
BioA) containing tobacco plants

| | pCMP MTP PaBioA | | | | | |
|---|---|---|---|---|---|---|
| | Compound L (600 gai/ha) | | | Compound A (600 gai/ha) | | |
| D4602 | 7 DAT | 14 DAT | 21 DAT | 7 DAT | 14 DAT | 21 DAT |
| 1332 | 0 | 0 | 0 | 2 | 0 | 0 |
| 1333 | 0 | 0 | 0 | 0 | 0 | 0 |
| 1334 | 45 | 85 | 90 | 15 | 60 | 60 |
| 1336 | 45 | 80 | 90 | 0 | 35 | 10 |
| 1337 | 0 | 0 | 0 | 0 | 0 | 0 |
| 1338 | 0 | 0 | 0 | 0 | 0 | 0 |
| 1339 | 30 | 70 | 80 | 10 | 40 | 60 |
| 1344 | 0 | 0 | 0 | 0 | 0 | 0 |
| 1345 | 0 | 0 | 0 | 0 | 0 | 0 |
| 1349 | 3 | 0 | 0 | 0 | 0 | 0 |
| 1355 | 0 | 0 | 0 | 0 | 0 | 0 |
| 1356 | 0 | 0 | 0 | 0 | 0 | 0 |
| 1362 | 0 | 0 | 0 | 0 | 0 | 0 |
| 1368 | 0 | 0 | 0 | 0 | 0 | 0 |
| 1374 | 35 | 75 | 90 | 5 | 40 | 60 |
| 1376 | 0 | 0 | 0 | 0 | 0 | 0 |
| 1379 | 0 | 0 | 0 | 0 | 0 | 0 |
| 1387 | 0 | 0 | 0 | 2 | 0 | 0 |
| 1403 | 0 | 0 | 0 | 0 | 0 | 0 |
| 1405 | 0 | 0 | 0 | 0 | 0 | 0 |

TABLE 18
results of percentage of damage to D4603 (Stenotrophomonas
maltophilia BioA) containing tobacco plants

| | pCMP MTP SmBioA | | | | | |
|---|---|---|---|---|---|---|
| | Compound L (600 gai/ha) | | | Compound A (600 gai/ha) | | |
| D4603 | 7 DAT | 14 DAT | 21 DAT | 7 DAT | 14 DAT | 21 DAT |
| 1492 | 0 | 0 | 0 | 0 | 0 | 0 |
| 1493 | 0 | 0 | 0 | 0 | 0 | 0 |
| 1495 | 30 | 75 | 90 | 20 | 60 | 50 |
| 1502 | 50 | 85 | 90 | 15 | 20 | 20 |
| 1504 | 35 | 80 | 90 | 10 | 40 | 50 |
| 1505 | 0 | 0 | 0 | 0 | 0 | 0 |
| 1507 | 0 | 0 | 0 | 0 | 0 | 0 |
| 1513 | 0 | 0 | 0 | 0 | 0 | 0 |
| 1518 | 45 | 85 | 90 | 5 | 40 | 55 |
| 1520 | 0 | 0 | 0 | 0 | 0 | 0 |
| 1523 | 0 | 0 | 0 | 0 | 0 | 0 |
| 1525 | 40 | 80 | 75 | 0 | 30 | 60 |
| 1527 | 0 | 0 | 0 | 0 | 0 | 0 |
| 1544 | 0 | 0 | 0 | 0 | 0 | 0 |
| 1556 | 0 | 0 | 0 | 0 | 0 | 0 |
| 1563 | 0 | 0 | 0 | 0 | 0 | 0 |
| 1565 | 50 | 80 | 85 | 10 | 50 | 65 |
| 1567 | 0 | 0 | 0 | 0 | 0 | 0 |
| 1568 | 50 | 80 | 85 | 10 | 40 | 55 |
| 1570 | 50 | 80 | 85 | 15 | 40 | 65 |

TABLE 19
results of percentage of damage to D4609 (Chroococcidiopsis
sp. CCMEE 29 BioA) containing tobacco plants

| | pCMP MTP ChrBioA | | | | | |
|---|---|---|---|---|---|---|
| | Compound L (600 gai/ha) | | | Compound A (600 gai/ha) | | |
| D4609 | 7 DAT | 14 DAT | 21 DAT | 7 DAT | 14 DAT | 21 DAT |
| 1575 | 40 | 75 | 85 | 5 | 30 | 25 |
| 1580 | 0 | 0 | 0 | 0 | 0 | 0 |
| 1586 | 0 | 0 | 0 | 0 | 0 | 0 |
| 1590 | 0 | 0 | 0 | 0 | 0 | 0 |
| 1592 | 0 | 0 | 0 | 0 | 0 | 0 |
| 1598 | 0 | 0 | 0 | 0 | 0 | 0 |
| 1599 | 0 | 5 | 3 | 0 | 0 | 0 |
| 1602 | 0 | 10 | 3 | 0 | 0 | 0 |
| 1624 | 55 | 80 | 90 | 0 | 30 | 40 |
| 1626 | 50 | 80 | 90 | 5 | 20 | 40 |
| 1627 | 0 | 0 | 0 | 0 | 0 | 0 |
| 1630 | 50 | 85 | 90 | 10 | 20 | 10 |
| 1631 | 40 | 80 | 90 | 0 | 35 | 60 |
| 1633 | 45 | 80 | 90 | 15 | 35 | 40 |
| 1634 | 0 | 0 | 0 | 0 | 0 | 0 |
| 1639 | 0 | 0 | 0 | 0 | 0 | 0 |
| 1641 | 0 | 0 | 0 | 0 | 0 | 0 |
| 1644 | 0 | 0 | 0 | 0 | 0 | 0 |
| 1647 | 0 | 0 | 0 | 0 | 0 | 0 |
| 1648 | 0 | 0 | 0 | 0 | 0 | 0 |

TABLE 20
results of percentage of damage to D4611 (Bacillus subtilis
BioA) containing tobacco plants

| | pCMP MTP BsBioA | | | | | |
|---|---|---|---|---|---|---|
| | Compound L (600 gai/ha) | | | Compound A (600 gai/ha) | | |
| D4611 | 7 DAT | 14 DAT | 21 DAT | 7 DAT | 14 DAT | 21 DAT |
| 1659 | 0 | 0 | 0 | 0 | 0 | 0 |
| 1661 | 0 | 0 | 0 | 0 | 0 | 0 |
| 1664 | 50 | 80 | 90 | 25 | 45 | 65 |
| 1666 | 60 | 0 | 0 | 0 | 0 | 0 |
| 1668 | 45 | 75 | 85 | 20 | 20 | 20 |
| 1670 | 0 | 0 | 0 | 0 | 0 | 0 |
| 1684 | 0 | 0 | 0 | 0 | 0 | 0 |
| 1689 | 40 | 80 | 90 | 0 | 30 | 10 |
| 1694 | 0 | 0 | 0 | 0 | 0 | 0 |
| 1695 | 30 | 60 | 80 | 5 | 5 | 5 |
| 1698 | 0 | 5 | 0 | 0 | 0 | 0 |
| 1703 | 0 | 0 | 0 | 0 | 0 | 0 |
| 1710 | 0 | 5 | 30 | 0 | 0 | 0 |
| 1711 | 0 | 0 | 0 | 0 | 0 | 0 |
| 1717 | 20 | 20 | 20 | 3 | 15 | 30 |
| 1720 | 40 | 80 | 90 | 5 | 15 | 5 |
| 1729 | 0 | 0 | 0 | 0 | 0 | 0 |
| 1730 | 0 | 5 | 5 | 0 | 0 | 0 |
| 1737 | 0 | 0 | 0 | 0 | 0 | 0 |
| 1738 | 0 | 0 | 0 | 0 | 0 | 0 |

TABLE 21
results of percentage of damage to D4606 (Streptomyces
viridochromogenes BioA) containing tobacco plants

| | pCMP MTP SvBioA | | | | | |
|---|---|---|---|---|---|---|
| | Compound L (600 gai/ha) | | | Compound A (600 gai/ha) | | |
| D4606 | 7 DAT | 14 DAT | 21 DAT | 7 DAT | 14 DAT | 21 DAT |
| 1910 | 10 | 0 | 0 | 0 | 0 | 0 |
| 1926 | 0 | 0 | 0 | 10 | 0 | 0 |
| 1928 | 0 | 0 | 0 | 0 | 0 | 0 |
| 1933 | 0 | 0 | 0 | 0 | 5 | 0 |
| 1934 | 2 | 0 | 0 | 0 | 10 | 30 |
| 1936 | 3 | 0 | 0 | 0 | 0 | 0 |
| 1939 | 0 | 0 | 0 | 0 | 0 | 0 |
| 1948 | 0 | 0 | 0 | 0 | 0 | 0 |
| 1950 | 0 | 0 | 0 | 0 | 0 | 0 |
| 1955 | 0 | 0 | 0 | 0 | 0 | 0 |
| 1956 | 0 | 0 | 0 | 0 | 0 | 0 |
| 1959 | 0 | 0 | 0 | 0 | 0 | 0 |
| 1969 | 2 | 0 | 0 | 0 | 0 | 0 |
| 1973 | 2 | 0 | 0 | 0 | 0 | 0 |
| 1975 | 0 | 0 | 0 | 0 | 0 | 0 |
| 1979 | 0 | 0 | 0 | 0 | 0 | 0 |
| 1988 | 0 | 0 | 0 | 0 | 0 | 0 |
| 1990 | 0 | 0 | 0 | 0 | 0 | 0 |
| 1991 | 0 | 0 | 0 | 0 | 5 | 0 |
| 1910 | 10 | 0 | 0 | 0 | 0 | 0 |

TABLE 22
results of percentage of damage to D4607 (Pedobacter hartonius
BioA) containing tobacco plants

| | pCMP MTP PhBioA | | | | | |
|---|---|---|---|---|---|---|
| | Compound L (600 gai/ha) | | | Compound A (600 gai/ha) | | |
| D4607 | 7 DAT | 14 DAT | 21 DAT | 7 DAT | 14 DAT | 21 DAT |
| 1996 | 0 | 0 | 0 | 0 | 0 | 0 |
| 1997 | 0 | 0 | 0 | 0 | 0 | 0 |
| 2010 | 50 | 85 | 90 | 20 | 20 | 5 |
| 2011 | 0 | 0 | 0 | 0 | 5 | 0 |
| 2012 | 0 | 0 | 0 | 0 | 0 | 0 |
| 2013 | 0 | 0 | 0 | 0 | 0 | 0 |
| 2019 | 50 | 80 | 90 | 20 | 60 | 65 |
| 2021 | 0 | 0 | 0 | 0 | 0 | 0 |
| 2028 | 0 | 0 | 0 | 0 | 0 | 0 |
| 2031 | 0 | 0 | 0 | 0 | 0 | 0 |
| 2036 | 0 | 0 | 0 | 0 | 5 | 0 |
| 2038 | 0 | 0 | 0 | 0 | 5 | 0 |
| 2052 | 0 | 0 | 0 | 0 | 0 | 0 |
| 2057 | 45 | 70 | 85 | 20 | 40 | 40 |
| 2058 | 0 | 0 | 0 | 0 | 0 | 0 |
| 2063 | 0 | 0 | 0 | 0 | 0 | 0 |
| 2065 | 0 | 0 | 0 | 0 | 0 | 0 |
| 2066 | 10 | 0 | 0 | 0 | 0 | 0 |
| 2068 | 0 | 0 | 0 | 0 | 0 | 0 |
| 2071 | 45 | 75 | 85 | 10 | 40 | 20 |

TABLE 23
results of percentage of damage to D4608 (Chitinophaga filiformis
BioA) containing tobacco plants

| | pCMP MTP CfBioA | | | | | |
|---|---|---|---|---|---|---|
| | Compound L (600 gai/ha) | | | Compound A (600 gai/ha) | | |
| D4608 | 7 DAT | 14 DAT | 21 DAT | 7 DAT | 14 DAT | 21 DAT |
| 2081 | 40 | 65 | 75 | 15 | 25 | 25 |
| 2091 | 0 | 0 | 0 | 0 | 0 | 0 |
| 2095 | 45 | 75 | 85 | 30 | 50 | 55 |
| 2096 | 0 | 0 | 0 | 0 | 0 | 0 |
| 2099 | 0 | 0 | 0 | 0 | 0 | 0 |
| 2106 | 50 | 80 | 85 | 45 | 60 | 65 |
| 2112 | 40 | 80 | 85 | 25 | 45 | 60 |
| 2115 | 0 | 0 | 0 | 0 | 0 | 0 |
| 2120 | 0 | 0 | 0 | 0 | 0 | 0 |
| 2122 | 0 | 0 | 0 | 0 | 0 | 0 |
| 2126 | 15 | 10 | 5 | 0 | 0 | 0 |
| 2138 | 0 | 0 | 0 | 0 | 0 | 0 |
| 2141 | 0 | 0 | 0 | 0 | 0 | 0 |
| 2145 | 45 | 85 | 85 | 30 | 30 | 25 |
| 2147 | 45 | 80 | 90 | 30 | 50 | 70 |
| 2150 | 0 | 0 | 0 | 0 | 0 | 0 |
| 2154 | 0 | 0 | 0 | 0 | 0 | 0 |
| 2155 | 0 | 0 | 0 | 0 | 0 | 0 |
| 2156 | 0 | 0 | 0 | 0 | 0 | 0 |
| 2159 | 0 | 0 | 0 | 0 | 0 | 0 |

TABLE 24
results of percentage of damage to D4610 (Tenacibaculum adriaticum
BioA) containing tobacco plants

| | pCMP MTP TaBioA | | | | | |
|---|---|---|---|---|---|---|
| | Compound L (600 gai/ha) | | | Compound A (600 gai/ha) | | |
| D4610 | 7 DAT | 14 DAT | 21 DAT | 7 DAT | 14 DAT | 21 DAT |
| 2163 | 50 | 85 | 90 | 35 | 70 | 80 |
| 2164 | 10 | 25 | 25 | 10 | 15 | 10 |
| 2171 | 0 | 0 | 0 | 0 | 5 | 0 |
| 2180 | 0 | 0 | 0 | 0 | 0 | 0 |
| 2192 | 0 | 0 | 0 | 0 | 0 | 0 |
| 2194 | 0 | 0 | 0 | 0 | 0 | 0 |
| 2198 | 0 | 0 | 0 | 0 | 0 | 0 |
| 2203 | 0 | 0 | 0 | 0 | 0 | 0 |
| 2204 | 50 | 85 | 90 | 40 | 70 | 75 |
| 2206 | 0 | 0 | 0 | 0 | 0 | 0 |
| 2207 | 45 | 80 | 90 | 25 | 50 | 50 |
| 2212 | 0 | 0 | 0 | 0 | 0 | 0 |
| 2215 | 50 | 85 | 90 | 40 | 60 | 65 |
| 2216 | 0 | 0 | 0 | 0 | 0 | 0 |
| 2217 | 10 | 0 | 0 | 0 | 0 | 0 |
| 2226 | 10 | 0 | 0 | 0 | 0 | 0 |
| 2231 | 0 | 0 | 0 | 0 | 0 | 0 |
| 2234 | 45 | 85 | 90 | 25 | 50 | 65 |
| 2238 | 0 | 0 | 0 | 0 | 0 | 0 |
| 2242 | 0 | 0 | 0 | 0 | 0 | 0 |

TABLE 25
Activity of WT BioA enzymes treated with DMSO or herbicide in vitro. Spermidine was
used in the substrate mix except where a * indicates that SAM was used, instead.

| | | Activity | | | | | | | | | | | |
|---|---|---|---|---|---|---|---|---|---|---|---|---|---|
| | SEQ | (%) | Compound L | | | Compound | Compound | Compound | Compound | Compound B | | | Compound |
| | ID | (vs. At | 0.5 | 31 | 500 | M | C | A | D | 31 | 200 | 500 | E |
| | NO | WT) | μM | μM | μM | 0.5 μM | 0.5 μM | 25 μM | 0.5 μM | μM | μM | μM | 200 μM |
| Arabidopsis | 294 | 100 | 83 | 99 | 98 | 45 | 53 | 96 | 95 | 79 | 96 | 96 | 96 |
| Escherichia coli BioA* | 159 | 120 | 4 | 8 | 35 | 0 | 8 | 6 | 6 | 4 | 9 | 23 | 12 |
| Pantoea ananatis BioA* | 167 | 136 | 9 | 0 | 23 | 11 | 11 | 7 | 6 | | 15 | | 15 |
| Streptomyces | 170 | 131 | 0 | 16 | 34 | 0 | 0 | 0 | 0 | | 0 | | 0 |
| viridochromogenes | | | | | | | | | | | | | |
| BioA* | | | | | | | | | | | | | |
| Cryptosporidium | 160 | 50 | 0 | | | NT | NT | NT | NT | | NT | | NT |
| andersoni BioA | | | | | | | | | | | | | |
| Stenotrophomonas | 171 | 216 | 0 | 0 | 16 | 1 | 0 | 0 | 76 | | 50 | | 74 |
| maltophilia BioA | | | | | | | | | | | | | |
| Chroococcidiopsis BioA | 184 | 50 | 15 | | | 10 | 2 | 0 | 0 | | 99 | | 26 |
| Pedobacter | 181 | 111 | 2 | | | 0 | 0 | 6 | 7 | | 96 | | 80 |
| hartonius BioA | | | | | | | | | | | | | |
| Chitinophaga | 180 | 3 | 11 | | | 0 | 12 | 0 | 0 | | 12 | | 28 |
| filiformis BioA | | | | | | | | | | | | | |
| Tenacibaculum | 185 | 9 | 16 | | | 13 | 19 | 14 | 5 | | 18 | | 16 |
| adriaticum BioA | | | | | | | | | | | | | |
| Bacillus subtilis BioA | 166 | 4 | 0 | | | 4 | 0 | 0 | 0 | | 0 | | 0 |

TABLE 26
Activity of WT and variant BioDA enzymes treated with DMSO or herbicide in vitro.

| | | Activity | % Inhibition relative to DMSO treatment | | | | | | | | | | | | | | |
|---|---|---|---|---|---|---|---|---|---|---|---|---|---|---|---|---|---|
| | SEQ | (%) | Compound L | | | Compound M | | | Compound | | Compound | Compound D | | Compound B | | | Compound |
| | ID | (vs. At | 0.5 | 31 | 500 | 0.5 | 31 | 500 | C | | A | 0.5 | 5 | 31 | 200 | 500 | E |
| | NO | WT) | μM | μM | μM | μM | μM | μM | 0.5 μM | | 25 μM | μM | μM | μM | μM | μM | 200 μM |
| Arabidopsis | 294 | 100 | 83 | 99 | 98 | 45 | | | 53 | | 96 | 95 | | 79 | 96 | 96 | 96 |
| BioDAΔMTP | | | | | | | | | | | | | | | | | |
| Schizo- | 13 | 191 | 0 | 55 | 94 | 15 | 95 | 99 | 17 | | 2 | 87 | | | 70 | | 28 |
| saccharomyces | | | | | | | | | | | | | | | | | |
| japonicus BioDA | | | | | | | | | | | | | | | | | |
| Ostreococcus tauri | 300 | 166 | 5 | 83 | 98 | 0 | 45 | 92 | 0 | | 0 | 0 | 36 | | 62 | | 53 |
| BioDAΔMTP | | | | | | | | | | | | | | | | | |
| Nannochloropsis | 3 | 105 | 6 | | | 3 | | | 8 | 5 | 5 | 14 | | | 92 | | 66 |
| gaditana BioDA | | | | | | | | | | | | | | | | | |
| Selaginella | 320 | 2 | 9 | | | 0 | | | 9 | 25 | 25 | 75 | | | 64 | | 69 |
| moellendorcffii | | | | | | | | | | | | | | | | | |
| BioDAΔMTP | | | | | | | | | | | | | | | | | |
| Quercus robur | 302 | 71 | 83 | | | 22 | | | 32 | 72 | 72 | 88 | | | 93 | | 96 |
| BioDAΔMTP | | | | | | | | | | | | | | | | | |
| Ostreococcus | 321 | 19 | | 98 | 93 | | 94 | 93 | | | | 82 | 95 | | 95 | | |
| tauri BioDA A374D | | | | | | | | | | | | | | | | | |
| Ostreococcus | 322 | 9 | | 4 | 53 | | 7 | 46 | | | | 0 | 7 | | 66 | | |
| tauri BioDA F332A | | | | | | | | | | | | | | | | | |
| Ostreococcus | 323 | 176 | | 58 | 94 | | 37 | 92 | | | | 4 | 26 | | 46 | | |
| tauri BioDA P407A | | | | | | | | | | | | | | | | | |

TABLE 27
IC50 of Arabidopsis thaliana BioDA variant enzymes.

| Variant/Mutant | SEQ | | IC50 | | | |
|---|---|---|---|---|---|---|
| with respect to | ID | Activity | Compound | Compound | Compound | Compound |
| SEQ ID NO: 1 | NO | (%) | L (nM) | B (nM) | K (μM) | G (μM) |
| WT | 294 | 100 | 145 | 2814 | 25 | 89 |
| C388D | 358 | 3 | 100100 | 29180 | 386 | 361 |
| C388T | 359 | 175 | 92 | | | |
| F348C | 360 | 15 | 77570 | | | |
| P421L | 373 | 34 | 687 | | | |
| W391L | 398 | 5 | 16280 | | | |
| W392G | 401 | 9 | 17660 | | | |

TABLE 28
Plants transformed with mutant BIO3-BIO1 genes.

| DNA | | | SEQ |
|---|---|---|---|
| ID | Name | Gene background | ID NO |
| D4581 | pCMP AtBioDA C388D | Arabidopsis thaliana | 342 |
| D4585 | pCMP AtBioDA P421L | Arabidopsis thaliana | 488 |
| D4596 | pCMP AtBioDA F348C | Arabidopsis thaliana | 344 |
| D4597 | pCMP AtBioDA G700S | Arabidopsis thaliana | 345 |
| D4604 | pCMP AtBioDA W392S | Arabidopsis thaliana | 343 |
| D4586 | pCMP ZmBioDA C372D | Zea mays | 346 |

TABLE 29
results of percentage of damage to WT tobacco plants

| | Wildtype Samsun tobacco | | | | | |
|---|---|---|---|---|---|---|
| | Compound L (600 gai/ha) | | | Compound A (600 gai/ha) | | |
| WT | 7 DAT | 14 DAT | 21 DAT | 7 DAT | 14 DAT | 21 DAT |
| 1 | 55 | 80 | 85 | 5 | 30 | 35 |
| 2 | 50 | 75 | 75 | 0 | 20 | 20 |
| 3 | 50 | 80 | 90 | 10 | 40 | 70 |
| 4 | 55 | 80 | 90 | 25 | 45 | 65 |
| 5 | 40 | 80 | 90 | 20 | 35 | 50 |
| 6 | 45 | 80 | 75 | 5 | 20 | 10 |
| 7 | 50 | 80 | 90 | 10 | 50 | 60 |
| 8 | 50 | 85 | 90 | 5 | 35 | 55 |

TABLE 30
results of percentage of damage to D4581 (Arabidopsis thaliana
BIO3-BIO1 C388D) containing tobacco plants

| | pCMP AtBioDA C388D | | | | | |
|---|---|---|---|---|---|---|
| | Compound L (600 gai/ha) | | | Compound A (600 gai/ha) | | |
| D4581 | 7 DAT | 14 DAT | 21 DAT | 7 DAT | 14 DAT | 21 DAT |
| 0654 | 0 | 0 | 0 | 0 | 0 | 0 |
| 0656 | 25 | 25 | 0 | 0 | 0 | 0 |
| 0658 | 0 | 0 | 0 | 0 | 0 | 0 |
| 0661 | 40 | 60 | 80 | 10 | 20 | 5 |
| 0662 | 0 | 0 | 0 | 0 | 0 | 0 |
| 0663 | 50 | 30 | 10 | 10 | 10 | 0 |
| 0664 | 25 | 30 | 10 | 5 | 5 | 2 |
| 0666 | 40 | 75 | 80 | 20 | 30 | 15 |
| 0685 | 35 | 60 | 80 | 5 | 20 | 10 |
| 0686 | 20 | 30 | 20 | 0 | 5 | 0 |
| 0694 | 0 | 0 | 0 | 0 | 10 | 10 |
| 0698 | 0 | 5 | 0 | 0 | 0 | 0 |
| 0699 | 0 | 0 | 0 | 0 | 0 | 0 |
| 0702 | 25 | 75 | 80 | 5 | 10 | 5 |
| 0703 | 0 | 0 | 0 | 3 | 0 | 0 |
| 0704 | 0 | 0 | 0 | 0 | 0 | 0 |
| 0714 | 0 | 0 | 0 | 0 | 0 | 0 |
| 0727 | 10 | 0 | 0 | 0 | 0 | 0 |
| 0731 | 0 | 0 | 0 | 0 | 0 | 0 |
| 0734 | 30 | 50 | 85 | 5 | 10 | 2 |

TABLE 31
results of percentage of damage to D4585 (Arabidopsis thaliana
BIO3-BIO1 P421L) containing tobacco plants

| | pCMP AtBioDA P421L | | | | | |
|---|---|---|---|---|---|---|
| | Compound L (600 gai/ha) | | | Compound A (600 gai/ha) | | |
| D4585 | 7 DAT | 14 DAT | 21 DAT | 7 DAT | 14 DAT | 21 DAT |
| 0748 | 15 | 10 | 5 | 0 | 0 | 0 |
| 0750 | 10 | 5 | 2 | 0 | 0 | 0 |
| 0751 | 35 | 55 | 85 | 5 | 10 | 5 |
| 0753 | 30 | 70 | 85 | 5 | 5 | 5 |
| 0754 | 20 | 10 | 5 | 3 | 0 | 0 |
| 0756 | 30 | 55 | 75 | 15 | 5 | 2 |
| 0764 | 25 | 10 | 5 | 0 | 0 | 0 |
| 0772 | 15 | 0 | 5 | 0 | 0 | 0 |
| 0773 | 15 | 5 | 5 | 0 | 0 | 0 |
| 0782 | 20 | 15 | 10 | 0 | 0 | 0 |
| 0783 | 60 | 70 | 65 | 5 | 15 | 0 |
| 0791 | 40 | 15 | 5 | 0 | 0 | 0 |
| 0792 | 20 | 5 | 0 | 0 | 0 | 0 |
| 0799 | 25 | 5 | 5 | 0 | 0 | 0 |
| 0801 | 20 | 10 | 5 | 0 | 0 | 0 |
| 0804 | 20 | 5 | 0 | 0 | 0 | 0 |
| 0806 | 15 | 10 | 5 | 5 | 0 | 0 |
| 0814 | 35 | 60 | 85 | 15 | 10 | 5 |
| 0816 | 15 | 5 | 0 | 0 | 0 | 0 |
| 0820 | 15 | 5 | 3 | 0 | 0 | 0 |

TABLE 32
results of percentage of damage to D4596 (Arabidopsis thaliana
BIO3-BIO1 F348C) containing tobacco plants

| | pCMP AtBioDA F348C | | | | | |
|---|---|---|---|---|---|---|
| | Compound L (600 gai/ha) | | | Compound A (600 gai/ha) | | |
| D4596 | 7 DAT | 14 DAT | 21 DAT | 7 DAT | 14 DAT | 21 DAT |
| 0987 | 0 | 0 | 0 | 0 | 0 | 0 |
| 0999 | 0 | 0 | 0 | 0 | 0 | 0 |
| 1004 | 40 | 70 | 80 | 0 | 50 | 65 |
| 1006 | 0 | 0 | 0 | 0 | 0 | 0 |
| 1009 | 0 | 0 | 0 | 0 | 0 | 0 |
| 1010 | 0 | 0 | 0 | 0 | 0 | 0 |
| 1019 | 0 | 0 | 0 | 0 | 0 | 0 |
| 1029 | 0 | 0 | 0 | 0 | 0 | 0 |
| 1030 | 0 | 0 | 0 | 0 | 0 | 0 |
| 1031 | 0 | 0 | 0 | 0 | 0 | 0 |
| 1033 | 0 | 0 | 0 | 0 | 0 | 0 |
| 1034 | 0 | 0 | 0 | 0 | 0 | 0 |
| 1041 | 0 | 0 | 0 | 0 | 0 | 0 |
| 1042 | 0 | 0 | 0 | 3 | 0 | 0 |
| 1047 | 0 | 0 | 0 | 0 | 0 | 0 |
| 1049 | 10 | 15 | 5 | 0 | 5 | 0 |
| 1052 | 0 | 0 | 0 | 0 | 0 | 0 |
| 1057 | 8 | 45 | 50 | 0 | 5 | 2 |
| 1061 | 0 | 0 | 0 | 0 | 0 | 0 |
| 1062 | 0 | 0 | 0 | 0 | 0 | 0 |

TABLE 33
results of percentage of damage to D4597 (Arabidopsis thaliana
BIO3-BIO1 G700S) containing tobacco plants

| | pCMP AtBioDA G700S | | | | | |
|---|---|---|---|---|---|---|
| | Compound L (600 gai/ha) | | | Compound A (600 gai/ha) | | |
| D4597 | 7 DAT | 14 DAT | 21 DAT | 7 DAT | 14 DAT | 21 DAT |
| 1071 | 0 | 0 | 0 | 0 | 0 | 0 |
| 1076 | 10 | 0 | 0 | 0 | 0 | 0 |
| 1079 | 40 | 75 | 80 | 5 | 30 | 30 |
| 1091 | 0 | 0 | 0 | 0 | 0 | 0 |
| 1096 | 0 | 0 | 0 | 0 | 0 | 0 |
| 1100 | 0 | 0 | 0 | 0 | 0 | 0 |
| 1106 | 0 | 0 | 0 | 0 | 0 | 0 |
| 1108 | 0 | 0 | 0 | 0 | 0 | 0 |
| 1112 | 0 | 0 | 0 | 0 | 0 | 0 |
| 1113 | 45 | 65 | 75 | 15 | 40 | 30 |
| 1115 | 0 | 0 | 0 | 0 | 0 | 0 |
| 1118 | 20 | 10 | 15 | 0 | 5 | 0 |
| 1119 | 0 | 0 | 0 | 0 | 0 | 0 |
| 1122 | 0 | 0 | 0 | 0 | 0 | 0 |
| 1126 | 35 | 75 | 85 | 15 | 50 | 65 |
| 1139 | 10 | 5 | 5 | 0 | 0 | 0 |
| 1140 | 0 | 0 | 0 | 0 | 0 | 0 |
| 1141 | 0 | 0 | 0 | 0 | 0 | 0 |
| 1142 | 0 | 0 | 0 | 0 | 0 | 0 |
| 1151 | 0 | 0 | 0 | 0 | 0 | 0 |

TABLE 34
results of percentage of damage to D4604 (Arabidopsis thaliana
BIO3-BIO1 W392S) containing tobacco plants

| | pCMP AtBioDA W392S | | | | | |
|---|---|---|---|---|---|---|
| | Compound L (600 gai/ha) | | | Compound A (600 gai/ha) | | |
| D4597 | 7 DAT | 14 DAT | 21 DAT | 7 DAT | 14 DAT | 21 DAT |
| 1411 | 45 | 70 | 75 | 25 | 50 | 30 |
| 1413 | 0 | 0 | 0 | 20 | 0 | 0 |
| 1424 | 40 | 70 | 80 | 0 | 50 | 70 |
| 1435 | 20 | 20 | 40 | 5 | 20 | 20 |
| 1436 | 20 | 35 | 65 | 15 | 40 | 40 |
| 1439 | 0 | 0 | 0 | 0 | 0 | 0 |
| 1440 | 0 | 0 | 0 | 0 | 0 | 0 |
| 1441 | 3 | 0 | 0 | 5 | 5 | 0 |
| 1442 | 0 | 0 | 0 | 5 | 0 | 0 |
| 1443 | 0 | 0 | 0 | 0 | 0 | 0 |
| 1450 | 35 | 70 | 80 | 10 | 35 | 35 |
| 1452 | 0 | 0 | 0 | 0 | 0 | 0 |
| 1458 | 0 | 0 | 0 | 0 | 0 | 0 |
| 1460 | 0 | 0 | 0 | 0 | 0 | 0 |
| 1467 | 0 | 0 | 0 | 3 | 0 | 0 |
| 1470 | 15 | 10 | 5 | 5 | 15 | 20 |
| 1474 | 0 | 0 | 0 | 3 | 0 | 0 |
| 1482 | 30 | 65 | 80 | 10 | 40 | 70 |
| 1483 | 30 | 50 | 70 | 2 | 35 | 40 |
| 1486 | 30 | 80 | 90 | 8 | 30 | 25 |

TABLE 35
results of percentage of damage to D4586 (Zea mays
BIO3-BIO1 C372D) containing tobacco plants

| | pCMP ZmBioDA C372D | | | | | |
|---|---|---|---|---|---|---|
| | Compound L (600 gai/ha) | | | Compound A (600 gai/ha) | | |
| D4586 | 7 DAT | 14 DAT | 21 DAT | 7 DAT | 14 DAT | 21 DAT |
| 0904 | 5 | 0 | 0 | 0 | 0 | 0 |
| 0905 | 15 | 0 | 0 | 0 | 0 | 0 |
| 0906 | 25 | 15 | 5 | 0 | 0 | 0 |
| 0909 | 5 | 0 | 0 | 0 | 0 | 0 |
| 0915 | 5 | 0 | 0 | 0 | 0 | 0 |
| 0916 | 30 | 60 | 85 | 25 | 25 | 20 |
| 0919 | 5 | 0 | 0 | 0 | 0 | 0 |
| 0921 | 5 | 0 | 0 | 0 | 0 | 0 |
| 0924 | 5 | 0 | 0 | 0 | 0 | 0 |
| 0925 | 25 | 25 | 10 | 0 | 0 | 0 |
| 0926 | 25 | 0 | 0 | 0 | 0 | 0 |
| 0929 | 5 | 0 | 0 | 0 | 0 | 0 |
| 0932 | 5 | 0 | 0 | 0 | 0 | 0 |
| 0935 | 35 | 65 | 85 | 15 | 20 | 5 |
| 0936 | 5 | 0 | 0 | 5 | 0 | 0 |
| 0939 | 5 | 0 | 0 | 0 | 0 | 0 |
| 0941 | 10 | 3 | 0 | 0 | 0 | 0 |
| 0942 | 15 | 5 | 0 | 0 | 0 | 3 |
| 0947 | 10 | 3 | 0 | 0 | 0 | 0 |
| 0951 | 20 | 5 | 5 | 0 | 0 | 0 |

SEQUENCES

| SEQ | | |
|---|---|---|
| ID NO | | |
| | BioDA/BIO3-BIO1 | |
| | protein sequences- | |
| | full length | |
| 1 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein (SEQ ID | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | NO: 1) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 2 | Zea mays BioDA protein | MVRRLFLHHARRRLHSGTASSIPLSTPIFAIFGANTGVGKTLVSAGLATALLSSPSPAVSSVAYLKPLQTGYPVDSDASFVFSRTPALLRAFPPC |
| | sequence (SEQ ID | RATRLIASCRTFFQSPAVEAKAESLHESQEKVVTYGAGAAEQTKVLACCTAYAWREPVSPHLAAEREGMAAGDDEVKGCVEQWLLEEGIGE |
| | NO: 2) | GGEVWKILETAGGVASPSASGTLQCDLYRPFRLPAILVGDGRLGGISSTLSAYETLLLRGYDVSAVILEDRELSNDKFLLSYLRNRVHVLILPQI |
| | | PEDPLDDLTDWFSESSSIFIFLKDELQSFHSRRIERLNSMQRKSKALLWWPFTQHNLVPQDSVTVIDSRYGENFSVYKIKDKMLIPQFDACAS |
| | | WWTQGPDSNLQIELARDMGYAAARYGHVMFPENVHEPALHSAEVLLGGVGKGWASRVYYSDNGSTAIEIALKMAFRKFSLDHGIMASSENS |
| | | TRNERNIQLKVLALNGSYHGDTLGAMEAQAPSAYTSFLQQPWYSGRGLFLDPPTVYIKSEVYNISLPQSMQHDPQTYGFFSQAEVFCKTRDK |
| | | TFAADLYSSYIKQKLSEFSLSSSSEHLAALIIEPVIQGAGGMLMIDPLFQRVLVSECRSRKIPVIFDEVFTGFWRLGVESASELLGCLPDIACYAK |
| | | LMTGGIIPLAATLATEEVFESFESDSKLTALLHGHSYTAHAMGCTAALKAMQWYRGPSTNSNLDTDHMKLKELWDGTLVKQLSSLPNVKRVV |
| | | SLGTLCAIELKAEGADAGYASLYASSLVQQLRKEDDIYIRPLGNVIYLMCGPCTPRDSCTRQLLKVHRRLCALN* |
| 3 | Nannochloropsis | MCSLTIQVFGANTDVGKTVISAGLLRAASNRPLRRRTTYIKPLQTGKIHDGSFVQRYTSADVSCHTLYSWETPLSPHVAATKEGKTISSEELLG |
| | gaditana BioDA protein | MLRTCLQQAQESHTVGGDGGGVRQREGSNVGDLVVVETAGGVLSPGPSPLQTQADLYRPLRLPVLLVGDGRLGGISSTLAAYESLYCRGY |
| | (SEQ ID NO: 3) | EVHAIAVLEVPHYIENAAAVSEYITGTLGHAETPVLSLPHLPEVSSVSFSTGDVDKDVLLGGLDQWYHQHEHVFHHLLDTLIDRERVRIQRLQK |
| | | MTTQAAQVFWYPFTQHSGLSGTDLHVVDSAYGDNYTLISKSSEDGDAKERVPCSRTLIDACASWWTQGLGHGRVDVALAVGAAAGRFGHVI |
| | | FPGNVHEPALRLAELMLAGPGQGWASRVFFSDNGSTAMEIAVKMALKKNSVDLKYEKGCSKEEFRVEQPNDKISATPLTVVTQRDCYHGDT |
| | | LGCMDAAAPTIFNTGQHPWYQPRTLSLTVPTFGYVSGHLQVEIPPEITRAAGQSALQLPDSGQVLKFPGGLATLLDVHAREGSDAAVIYRKAI |
| | | RAQFIAFERGEEGILSSNTVIPLRGEAGQGHRCRKLGAVLLEPVLMGAAGMVMVDPLFQRILTLEGRAYGLPVVFDEIASGLYRLGCASAAELL |
| | | GVAPDIACYGKTLTGGYLPMALTLTTEAVFEAFHGTNKSDALLHGHSYTANPLGCAAAVHALKAYAVIHETNIRTSQECCQRKEDFFPNKNKC |
| | | SSHHNICRYNEDQVAALSNLSGVRRAVALGTVLAVELTEVESSNRKGYSSTASTHIVNDLREAGIYARPLGNVVYLIASQVSSGGTFECLVDTL |
| | | DRVLRRRGDTLGKFTHDMTTSGVVI* |
| 4 | Taxus chinensis BioDA | MASLALTRRRFNPVSRLSLHKCRSTFSRSGQAGEDRTPKQKLHIPSGFEVDLSHPTYVIWGSNTGVGKTLVSTGLAASLLNASQNLLYVKPV |
| | protein (SEQ ID NO: 4) | QTGFPQDSDSRYVSHKVKDILKQRKPSNNVHISHQVLKASPRAWELLPGSKGFGEVASYERAIQGMRNVCDYEGQTVQGLYKGLADLKLVC |
| | | KTIYGWYEAVSPHLAAAGEKCSVDDCEVVENLQNSLREFNVDENNTNRWSIIETAGGVASPGPSGTLQCDLYRPFRLPSILVGDGRLGGISG |
| | | TISAYETLCIRGYDICAVILADTGLSNEKCLSTYLHDRVPVFVLPKIPDDTNNTLLEWFSESYKVFSEIKDTLQTSYFKRIYRLHEMPMKARQILW |
| | | WPFTQHSLVTEQSISLIDSRFGENFGVYKGKNHKDVKESIVQQFDACASWWTQGPSAILQTELAREVGYTTGRFGHVMYPENVYEPALHCA |
| | | ELLLEGVGKGWATRVFLSDNGSTAIEISLKMAFRKFAFDHGFLPEHFNEDLRKICPQFKVEIGTISYTTCAFFIEMTMEEFSEESYTGRGLFLDP |
| | | PTVFLHNEEWQLHLPDALMYSDLKYPGLVIHASGGMHMIDPLFQRDLVKECRKRRIPVIFDEVFTGFWRLGAESAAEILGCAPDIACYAKLLTG |
| | | GIVPLAATLATESIFEAFEGKSKLFALLHGHSYSGHAIGCAAAIKAIQWFKDAEMNLNICTDGSRLQELWDSKLVAKISALSSVKRVIALGTICALE |
| | | LQADESNTGYGSLLASSLVQKLRDDGVYMRPLGNVIYLMCGPATPPAMCSTLLQKLHKRLDEC |
| 5 | Physcomitrium patens | MHLLLLLPLRRRCTNPIAPRIAHQSRFLVSTAGACSPLPRHLLSGIWGRCLSSHPSPIEDADEARCVAENVADALDVSYPTYMVWGSNTGVGK |
| | BioDA protein (SEQ ID | TLVSAGLCSAILRLSKCQGNSGAKDLLYLKPVQTGFPSDSDARYVYQEVSRVGRLHPEAISEGLFLCNHTLQVSPAVQISARAGASVPVQPQS |
| | NO: 5) | AWNAGGLSSSLNGTGMKDFCSYESQLLQGVKSRNIPADGDVYGRAQLRNTLSRFTCKTMWAWYEPISPHLAAAREGSTVPDTMVVEAIQR |
| | | SLQALSSSRKTSGHREDAGDCAEKWAILETAGGVASPGPSGSLQCDMYRSLRFSGLLVGDGRLGGISTTIAAYESLRSRGYDIPAIVIVDSGL |
| | | SNEEPVLQYLRHKIPVVVLPPLPEDPNKRLEEWFRLAEGTFLELMNVLENSHSKRLRRLEEMPKMAGEILWWPFTQHDLVAQDSITLIDSRSG |
| | | ENFSVYKSGTPSTGKTIATQFDACASWWTQGPDSILQPEIAREVAYTAGRYGHVMFPENVHEPALRCAEILLAGAGRGWAERVFFSDNGSTA |
| | | IEVAIKMAFRKYIVDHGLSDPSTIERRGGQLKVVALKGSYHGDTLGAMEAQAPSAYTGFKQQPWHVGRGFFLEPPTVFQRQGRWHLQLPRV |
| | | FHDSDSSDSASCWKSRDEIFDICRDSTPLANLYIRSINQQLAAAVREENNVPAALIIEPVMHGAGGMDMIDPLYQRMLVRECRRRGIPTIFDEV |
| | | LSGCWRFGVESAVDLLGCSPDIACYSKLLSGGLVPLAATLATQAVFEAFRGISKLDALLHGHSYTAHAVGCASAVVSLQYFSDPSKNSNLLPG |
| | | GRRLRELWKPELVSLISSHQSVERVVSVGTIFALELRANAAESGYASMLSKELVENLRQEGIYTRPLGNVVYLMCGPLTQPKTCTTMLEKLLAL |
| | | LG |
| 6 | Adiantum nelumboides | MAPPLSHLYPHLRIHQIFGANTDVGKTIFTTALALASAALPIGSKNGKIERDGQGSLNFQENGEAVHYIKPISTGPLRDSDSLHIARYLPSVNAHT |
| | BioDA protein (SEQ ID | LFQFNDPVSPHVAAARYKAEDKNKQDLDVSDEAVIHALSNKISTFTQAAIRPGAAYIETAGGVHSPAPSGSSISQLLRPLRMPTILIGDSNLGGI |
| | NO: 6) | STTKSAYDSLLMAGYDIEALLLFSDAGRGWGNAEYLTKWGEEIGLPVWALTGPSSSTVWGLPPAREGSDEEDARRMQEYYAGLVAGRGDA |
| | | AENDESSLGAAPIIEHLRRRHEKRLENIGSMAKRTRDTCWWPFTQHMLAKSDEDVNVIDSAYGDFFTVLNQSSENASKHGNTSLLRPLLDGS |
| | | ASWWTQCLGHADPGLTRAAARAAGRYGHVLFPMCANEPSLQLSETLVGKNSSDVIGTSPGEGWASRVFFSDDGSTGMEVALKMAISSASS |
| | | RYAPKAESQLAKSKVQQGRSAGNQSGRPPREWMVLGLKGSYHGDTIGAMDACEGNIFNEKVEWYRGRGSWFEPPTVSIKDGQAMVDLPL |
| | | QDADWQHLSGKVESKKNFNNVSDIYNVSDRMDNDELKNIYSKLIYGWLDHLTRMEGNRYGALILEPLIMGAGGMICVDPLFQRCFVDVIRANP |
| | | DLFSQVDPPLRGTSHIRESDVPGSGDWKGLPIIFDEVFTGLYRLGYSTPAQAIGVSPDISVLAKILTGGMVPMSVTLASDSIFSTFSKSDQKIDA |
| | | LLHGHSYTAHPIGCEIANEALRRIETMQVENFWGDAKADWESRTSSLQRDNSMKNVPSPWSLWSREAVEAFSRSANVESSMALGTVMILHL |
| | | SDSAGEKGYSSSAAVDILQHLRTTQDTGFNKHVSFNIHARPLGNVVYMMCSLNTPVNVLRETEAALKHVLKV |
| 7 | Setaria italica BioDA | MLPRLLLRSRHRRRYSSAAATVPLSTPTFAVFGANTGVGKTLVSAGLVAALLNSRSPSVSAVSYLKPLQTGYPADSDARFVFARAPALLRASS |
| | protein (SEQ ID NO: 7) | SPRATRLVASCRTLFPSPAVGPEAEPLHQSQEKVVTYGGDGAEEETKVLACRTVYAWREPVSPHLAAEREGMAAGDDEVRGCVDQWLME |
| | | EGVGEGGEVWKVLETAGGVASPSASGTLQCDLYRPFRLPAILVGDGRLGGISSTLSAYETLLLRGYDVSTVILEDRDLTNDTFLLSYLRNRVH |
| | | VLALPQIPEDPSDDLTDWFSESSSVFNLLKDSLQSFHSKRVERLNGMQKKSKDLLWWPFTQHNLVPQDSVTVIDSRCGENFSAYKIKDNTMM |
| | | LVPQFDACASWWTQGPDSDLQIELARDMGYAAARYGHVMFPENVHEPALRAAELLLGGVGKGWASRVYYSDNGSTAVEIALKMAFRKFSL |
| | | DHGILADSEKSIRNERNIQLKVLALNGSYHGDTLGAMEAQAPSAYTSFIQQPWYSGRGLFLDPPTVCIKSETCNVSLPQSMQHYPRTSGDTCF |
| | | PSLAEVFCKTRDTSSAADLYLSYIKQQLSEFSQSSNSDHLAALIIEPVIQGAGGMLMIDPLFQRVLVRECRSRKIPVIFDEVFTGFWRLGVESAS |
| | | ELLGCLPDIACYAKLMTGGIVPLAATLATEEVFESFKSDSKLTALLHGHSYTAHAMGCMAALKAIQWYRDPSTNSNLDLDHMKLKELWDGALV |
| | | KQLSSLPNVKRVVSLGTLCAIELKAEGSDAGYASLYASSLVQQLRREDDIYIRPLGNVIYLMCGPCTPRDSCSRQLHKVLRRLCDLN* |
| 8 | Phoenix dactylifera | MPSCPLLPLLPRRRPPPSPLLLLLLSRSLHSSPPELDLSHPTLVVWGSNTGVGKTIVSAGLAASVLSPSSPNTATFLYLKPVQTGFPRDSDSRF |
| | BioDA protein (SEQ ID | VYRKVSDLFRHRLGGFSAQLLASNHALKASVPAAREVLGARTERPGEEGEEASTGLCCYEETRVGTGESTTGDFKLVCKTLYGWKEAISPHL |
| | NO: 8) | AVEREGMAVEDSSLREVLGRLLNSSMGGGGDRRKENVWRVIETAGGVASPGPSGSIQCDLYRPFRLPAVLVGDGHLGGISTTLSAYESLTL |
| | | RGYDVIAIILADHGLSNEIPLLSNLRHRVPVLVLPPVPEDPLNDLVDWFCESSKAFSSLQDLVQSTHSKRIQRLHDMPRKAGNILWWPFTQHKL |
| | | VPEEIVTVIDSRCGENFAVHKVRNDREMIVRQFDACASWWTQGPDFALQIELARDMGYSAARYGHVMFPENVYEPALHCAELLLEGVGKGW |
| | | ASRTYYSDNGSTAIEIALKMAFRKFLLDNEMLMDSGNGISSERCIELRVLALNGSYHGDTLGAMEAQAPSSYTGFLQQPWYSGRGLFLDPPTI |
| | | FIHKEIWNLSVPESFESDQLKPEDMRFSSSDALFCPSRDSSAIAGIYSAYISNQLLQFSKLSCSTQIGALIIEPVIQAAGGMHMIDPLFQRILVREC |
| | | RNRRIPVVFDEVFTGFWRLGKESAAELLGCVPDVACFGKLMSGGIIPLAVTLTTEAVFEAFKGDSKLMALLHGHSYSAHAMGCTAAAKSIQWF |
| | | RDPFTNPNVEFKEKKLKELWEMKLVLQISSLPAVQRVITLGTLCAVELKAEGSDAGYASLYASLLVQQLREDGLYVRPLGNVIYLMCGPCTSS |
| | | HFCSQQLQQVYQRLHEFSQHQLRDSQSFQREG |
| 9 | Ostreococcus tauri | MSAPIARRASSVARGRTRWLTSTSIERSREWFVRSLGSASDGAASGNGRALELSIASFAIWGANTGVGKTLASAGLARAATRRGTATTFLKP |
| | ATCC 22665 BioDA | VQTGYPEDSDAGFVARQTKGNETAGEHAAVASGSTEGGGEGFAAGVTRAHTEFAWRRAAGPHLTAAEEGRPVSDEEIVRATRNYLREFAE |
| | protein (SEQ ID NO: 9) | SAREGNARGFAVIETAGGVASPGPSGTLQCEILRALRLPAILIGDGRLGGISTTTSAYESLHARGYDVVGVLMADDGYANHEAVAKILPSGTKV |
| | | FALPPLPPQGKADAWLEKSAGVFSEAFETAMNWHEKRIQALQKLPGEALEKIWWPFTQHATVEPDTVTVIDGRYGEDFEIYDQEKGRDGAID |
| | | LRFDGAASWWTQGVSAELQQELNSAAANAAGRYGHVMFPENVHQPSMDATNALLEGPGKGWGSRVFYSDNGSTATEVGLKMAFRKYYV |
| | | DAGLVSKDGHQRAKDLINDGSCHLPQLRIIALDGSYHGDTLGAMDMQSPSVFTGPMQTPWYQPRGLFLQPPSLAIRNGEWTVALPPHGLLN |
| | | DGEDRLFHDGKDLESNTTWKSKADAFDIGKRLDSPLANEYRQAVDKALERAEQDAKAGHMGPVGALMMEPVLHGAGGMVLIDPLFQNILMK |
| | | VCRERKIPIVLDEVFAGIWRLGTEGAWEIIGEKPDISCYAKLLTGGLMPMSATVATADVFDVFYGPGKAQGLLHGHSYTAYPIGCAVASKALSL |
| | | YKDPAKNPNLIVDASSKGDTPTAYLRELWDESKLKEISNKSSVRRVFGQGCVLAVELEVEGEGGYSSNAAKEVVLRLRPHSVQARPLGNVVY |
| | | LMCAPTTPKATCDRLLNVLAEELSE |
| 10 | Helianthus annuus | MFHPPLLFTLSRHFLRRHHHRHLSTLSHTLSHPIYTIWSANTGLGKTLVSAGIAAASVASPPSKLVYIKPIQTGYPLDSDAAFVYRKIPQILHRRN |
| | BioDA protein (SEQ ID | PNLTLTASNQTLNVSIPAAKVKIGDNFSDSRNGFGEFGFCEEMKVGEGVGEGSELICKTIYAWNEAISPHLASEREGALVGDEEVLVLLKKCLE |
| | NO: 10) | IGVGSEGQGSVLSLVETAGGVASPGSSGSLQCDLYRPFRLPSILVGDGKLGGISGTISAYESLTIRGYDVVAIVLEDHGLENEVPLLSYLRNRV |
| | | PVLVLPSIPKDPLDNLMEWYDESQSVFDSLKGIMLSSYEKQMYRLHEMPKKAQEIFWWPFTQHKLVPQEKVTVIDSRCGENFAVHKVKDHDY |
| | | ITQMFDACASWWTQGPNAALQIKLAREMGYTAARYGHVMFPENVYEPALQCAELLLQGVGKGWASRVFFSDNGSTAIEIALKMAFRKFLAD |
| | | NKLLMDLPHYNTDESNIELKVVALNGSYHGDTLGAMDAQAPSPYTGFLQQPWYTGRGVFLDPPFVSMCDGVWKIAVPKKMHYEDVILEDTS |
| | | FNSRDQVFNRSRDDSKLASIYSTYISQELLPSSGSTKCSYAGALIIEPVIQGAGGMLMVDPLFQRILVKECKNRKIPVIFDEVFTGYWRLGVESA |
| | | AELLFCQPDIACFAKLMTGGMIPLAATLATNDVFESFVGDSKLNALLHGHSYSAHAVGCATACNSIKWFKDSQTNPNLVPGQNLLRELWDAEL |
| | | VRKISFHPAVQRVVSLGTLFALELKVEDNDAGYASLSGTSLLLKLREDGIYMRPLGNVIYMMCGPCTSHRVCLQMLEKLYMRLDEFKQEKIGT |
| | | ARDYDHDHRLPIA |
| 11 | Quercus robur BioDA | MYFLISLHRRGHRHLRHVKDLSTTAHSQLHVPLNHPTFLVWASNTSLGKTLVSTGLAASSLLNPTPKPRKFLYLKPIQTGFPSESDSSSVFHKL |
| | protein (SEQ ID NO: 11) | SRLSLLRRHNLPHFSLFSSNHILKASFPAANSALGQEISETPHSGMRDLGYYEEAKPEGDVGGGAASQLVCKTLYAWREAVSPHLAAERESG |
| | | VVEDAAVLELLQKCLRFELEGGGEKGKETEIFCVVETAGGVASPGPSGSLQCDLYRPFRLPGILVGDGRLGGISGTISAYESLKLRGYDIAAVV |
| | | FEDHGLVNEMPLLSYLRNRVPVLVLPSVPKDLSNDLMEWFDESRGVFDSLKEEMLSAYSKRMMRLQDMPKKAGEIFWWPFTQHQLVPEEA |
| | | VTVIDSRHGENFAVFKAQNNEFITQQFDACASWWTQGPDTTLQTELARDMGYAAARFGHVMFPENVYEPALECAELLLEGVGKGWASRTFF |
| | | SDNGSTAIEIALKMAFRKFSYDHGILSDGRKDNTASQRVKLMVLALQGSYHGDTLGAMEAQAPSSYTGFLQQPWYTGRGLFLDPPTVYMCN |
| | | GVWKLSLPEGLHLEIPKLENKGFSSRDEIFHKIRDKSDLARIYSSYISEQLSQYSGSGGFYHIGALIMEPVILGAGGMHMIDPLFQRVLVNECRS |
| | | KKIPVIFDEVFTGFWRLGRETAAELLGCVPDIACFAKLMTGGIIPLAATLATNAIFNSFIGESKLKALLHGHSYSGHAMGCTAAVKSIKWFKDPQT |
| | | NLNITDEGRSLRELWDEDVVQQISSHPAVERVVALGTLFALELRAEGSNAGYASQYASSLLQNLRQDGIYTRPLGNVIYLMCGPCTSPEICSQ |
| | | LLVKLYKRLEDFDQFHQIREENSESYGG |
| 12 | Thraustotheca clavata | MLTLSMRMRPRTLMKMSFSTRAGLGQLGCLTSLDQSVPNAVPTSLHAPIVQVFGSNTDVGKTVIAAGLCRAALLSSKNATVGYIKPLQTGGD |
| | BioDA protein (SEQ ID | AMMDAEFLNRHAGNADNRLTSHTLFSWETAVSPHLAASLENKAVDDATVVAKLSKTLKQIQKEEKGSALTVVETAGGVCSPSASGKFQCDV |
| | NO: 12) | YRPLRLPTVLVGDGCLGGISATMSALDSLLIRGYDVSCIVLIEQDNLSNGEAISERASELGIPIFSLDKLPPQPEPLDLWYENSTETFRSVHDTIA |
| | | STHKARVARLQELPSIAKKALWWPFTQHKSNKGVLVIDSAYGDTFATYNQKNQAIEPMYDACASWWTQGVGHGNAKMALSLATAAGRYGH |
| | | VMFPENAHEPAIALSEMLLKSVGKNWAGKVFFSDDGSTAVEIGIKMALRKYAVDHGLGMSFEGINDVLVLAQEGCYHGDTLGTMNIAEPSVY |
| | | NKTQHPWYKPKALFVHPPSVKVASNGQMQVTWQNVNPTLVTHLPTLDDLFDISRDTSPLTQQYTEYVESLLDSVPSTTVVGALIVEPILIGAG |
| | | GMVFVDPLFQRTMVRVCQQRKIPVIFDEVFSGFWRLGAESARDLLQVNPDIACYAKLLTGGAVPMSTTLATNEVFDAFYTDSKADALLHGHS |
| | | FTANPVGCAAAVTALDMYKTIEKKNSTRVHWNVDQVAALAARPGIARAFQLGTVLVIELESTSRGYTDTTGQDVLVRLRNHGIYARSLGNVLYI |
| | | MASPLSTPATCIKIFNTVVDNLFA |
| 13 | Schizosaccharomyces | MLAHILFGANTEVGKTIWSTALCYASHYLAHAPVEYIKPVGAGPVEEADYVHLAKFVPCKCTNIHQFEKAMSPHMSEGAMQTTDASIVSALRK |
| | japonicus BioDA protein | HLQASSFKNTFVETAGGVSSPAPSGSLQCDLYRPLRLPVLLIGDSKLGGISTTIAAYEQLKVRGYDISAILMLRDPENAPQYENWKYFSEIWSK |
| | (SEQ ID NO: 13) | SYSCHDKENIKCFTIPSPPALSQDAEADFKAMQKYYSESASVGYDVLKHIEQVQKTRLARLDDMINQTEKHIWYPFTQHTLVNPKEIAVIDSAH |
| | | GDMYTVFAPNAKRKSEEALADGSKKEGSVAVEENKYKELFDASASWWTQGLGHGDPYLAMSAAYAAGRYGHVLLPNCTNEPALGLAEDLL |
| | | STVGKGWANRVYYSDNGATGMEIALKMALKAARIQHQLSGKIEIIGLEGAYHGDTIGTMDACPPNVYNVEVDWYDGRGIWFDSPVVKVSNGE |
| | | VSVQWEDKSFSFTALDDVYNVSSRVNSHLAKQYASFIGEKVRTAMQQGHQFGALVIEPLVVGAGGMLFVDPLFQRVLVDIAKGRLPESPWYT |
| | | EAHENKKAGIPVVFDEVFIGLYRLGQLRSADILGVYPDVQVNAKLLTGGLVPLSVTLASEELFEYFLSDKKSDALLHGHSYTAHPVGCHVARQS |
| | | LKRLDQLNRTAWTENKQRWLEFFGCQNVNGVLSSRNPNAWSVWDPEWVYEVSKLECVESIMALGSLASVVLKAGDHGYQSSAAGQYLSR |
| | | LREFSTGAEEYGILARPLGNVVYFMTSQITPTHTVKEIQAKIIDVFSQPR |
| 14 | Gibberella zeae BioDA | MAPVPALLWRSLRTHQVYGANTDVGKTIFSTVLCNAASKKGDRTWFLKPVSTGAVDEADGCHIQRFASSTNHETLFQYDIPCSPHIAAKVSG |
| | protein (SEQ ID NO: 14) | KPIPSDEAVLAKIHDSVSRYASEGPGWLFLETAGGVHSPGPSGTPQADLYTPLRAPVILVGDSKLGGISQTISAYESLRMRGHDIESILLFQDM |
| | | KYENYQYLKDYFSKQGGIPVDTVPEPPSRLPDIQQDTEQMREYYASQTSNVSHVLDHLDKRGKQRISRLESLSEKASKSIWYPFTQQKLVTA |
| | | DTISAIDSAHGDYFQVLNKTYENLLQPAFDGSASWWSQGLGHANSRLTLAAAYAAGRYGHVMFAEAIHEPALALAEMMLEGAQNSRFSRVF |
| | | YSDNGSTGCEVAVKMALRAARLRYGWGPNDNLQILGLKGSYHGDTIGAMDCAEPCVYNEKIEWYEGKGYWFDYPTIQCVEGKWVVSAPNG |
| | | MGQDQKLESISEVFNLESRLKTEQYRGYEEYIEKTLKKLQDQGRKFGALMMEPIILGAGGMIFVDPLFQRALVDVVRRSPHLFGTKDALPAQG |
| | | PQWSGLPVIFDEVFTGLYRLGRFTAASFLGTEADISVNAKLLTGGLVPLCTTMASESIFDAFKSDDKSDALLHGHSYTAHAVGCQVAVESVSE |
| | | MQSMEKNGEWEWAEKDWAEDETQAWSVWSRDFVTDMSHNPQVLGVWALGSVLAISLRDDDGVGYKSLAAKKIQSHLRQGTGSWNAHSR |
| | | VLGNVFYVMASQKTSRQSIRELQGLLVDALSK |
| 271 | Hordeum vulgare BIO3- | MLRRLLLRHARRLHYSSSFSSALPLSSPTFAVFGANTGVGKTLVSAGLAASLLRSHAASPSAVLYLKPLQTGFPDDSDAGFLYRRVPALLRPS |
| | BIO1 (SEQ ID NO: 271) | HAAHLAASIDTLSPAPSVETQSPSREEAAFSYDGEGEAVRKLLACRTLYAWREAVSPHLAAEREGMAVEDAHVRSLLAACLVEGENLGKGEG |
| | | DVWKLVETAGGVASPGPSGTLQCDLYRQFRLPTILVGDGRLGGISCTLSAYETLLLRGYDVSAVILEDHGLSNNKFLLSYLRNRVPVLVLPPIP |
| | | EDPSDDLTDWFSESSSVFGLLEDALRSFHLTRIQRLNSMQRKSKDLLWWPFTQHNLVPVDCVTVIDSRCGESFSAYKVKDNKMMMIPQFDA |
| | | CASWWTQGPDSKLQIELARDMGYAAARYGHVMFPENAHEPALRCAELLLGGIGKDWASRVYYSDNGSTAIEIALKMAFRKFSLDRGILLDSD |
| | | KSIITNGRNIQLKVLALKGSYHGDTLGAMEAQAPSAYTSFLQQPWYSGRGLFLDPPTVFIKNGTCTLSLPQAIQNCHLSPGDKCFPSLAEVFCK |
| | | TRDSSSAADLYSTYISQQLSEYSLSSNTEHIAALIIEPVIQGAGGMHMIDPLFQRVLVHECRDRKIPVIFDEVFTGFWRLGVESASELLGCLPDV |
| | | ACYAKLMTGGLVPLAATVTTEAVFEAFKSDSKLTALLHGHSYTAHAMGCSAAVKAIQWFRDPSTNSNLDFDCMKLKELWDGTLVNQLSSLPN |
| | | VKRIVSLGTLCAIELQAEGSDAGYASLYARSLIQQLREEDDIYVRPLGNVIYLMCGPCTTRDVCTEQLSKVYRRISHFNPVHRG |
| 272 | Brassica napus BIO3- | MSPVTATLLRHRLRHLRHHHIRLNSTAVPSHFNLPLNHPTYLIWSANTSLGKTLVSTGIASSFLLHQPSSSPPAAHSAKLLYLKPIQTGFPSDSD |
| | BIO1 (SEQ ID NO: 272) | SRFVFSKLDSLSLRRRIPLSVSNSVLRSSLPVAESMRRNIKVSESGMCDLNFREEKTVTGAPELLCKTLYAWEAAISPHLAAEREHATVEDSVV |
| | | LKMVEQEMECGSKANVLCLVETAGGVASPGPSGTLQCDLYRPFRLPGILVGDGRLGGISGTIAAYESLKLRGYDVAAVVFEDHGLVNEVPLT |
| | | SYLRNKVPVLVLPPVPKDPSDDLIEWFVESDGVFKALKEVMVSVYLERVERLNGMAKQAGEVFWWPFTQHKLVPEDNVTIIDSRCGENFSVF |
| | | KASDNNSITQQFDACASWWTQGPDPAFQAELAREMGYTAARFGHVMFPENVYEPALKCAELLLDGVGKGWASRVYFSDNGSTAIEIGLKMA |
| | | FRKFCVDHETLFGLSEDAEEKKHVDVKVLALRGSYHGDTLGAMEAQAPSPYTGFLQQPWYTGRGLFLEPPTVFLSNGTWNLSLPESFSQTA |
| | | SEECGTFTTRDEIFDKSRDTSVLATVYSTYVSEQLQEYSGISQSAHVGALIIEPVIHGAGGMHMVDPLFQRVLVNECRNRKIPVIFDEVFTGFW |
| | | RLGVETTADLLGCKPDIACFAKLLTGGMIPLAVTLATDAVFDSFSGDSKLKALLHGHSYSAHAMGCATATKAIEWFKDPETNHNIDSQRGTLRE |
| | | LWDEELVQHISCHCAVERVVVLGTLFALELKVDASNSGYASLYAKSLLEMLREDGIFMRPLGNVVYLMCGPCTSPEICRRLLTKLHKRLGEFN |
| | | RA |
| 273 | Gossypium hirsutum | MLLRRHHHHRLFRLRLLLLHHHHHQFQSFSTLTIPLSHPTYIIWSSNTSLGKTLVSTGLSSSFLLSPSSSSKKFLYLKPLQTGFPSDSDSRFLFQ |
| | BIO3-BIO1 (SEQ ID NO: | KLSSLSLSRNLPLFSSHSVLLSSLPAAKSFKPNEFSLNESREMCELGFYEEKKVLEAGRVAPELVSETIYAWEGALSPHFAAEREGGAVEDSE |
| | 273) | LVKRLETRLKEGLLEGGVERGKLDGFCVVETAGGVASPGPSGTLQCDLYRPLRFPGVLVGDGRLGGISGTISAYESLKLRGYDVVAIVIEDHG |
| | | LINEIPLKSYLRSRVPVFVLPPIPQDPSNDLMEWFDESCNVFNSLKDIMLEAYLERMRRLNEMPRKAGDVFWWPFTQHKLVPPSTVTVIDSRC |
| | | GENFSVYKVQNKEYITQQFDACASWWTQGPDATLQTELARDMGYAAARFGHVMFPENVYEPALECAELLLDGVGKGWATRVYFSDNGSTA |
| | | IEIALKMAFRKFSSDHGILPELLKNNPTESCTELMVLALKGSYHGDTLGAMEAQAPSSFTGFLQQPWYTGRGLFLDPPTVFMHNGKWTVSLP |
| | | EVFHSATLKPEDTTFRSRDEIFLKSRDESNLAGLYSLYVSQQLSQYSTLSETNKQIGALIMEPIIQGAGGMHMVDPLFQRMLVNECRHCRIPVIF |
| | | DEVFTGFWRLGVESAAELLGCVPDIACFAKLMTGGIIPLAATMATDAIFDSFTGDSKLKALLHGHSYSAHAMGCTAAAKSIKWFKDPNTNLNIT |
| | | SEKSQKMLLRELWDAELVQQFSSHPSISRVVTLGTLFALELQADESDAGYASLYARSLVQMLREDCIYTRPLGNVIYVMCGPCTSPKMCTLQL |
| | | LKLYTKLEEFTLVKTKMNII |
| 274 | Oryza sativa BIO3-BIO1 | MLRLLRHARRHSTSSSSSAAAAAVPLTSPAFAVFGANTGVGKTLVSAGLVASLLASPSPSPSTVAYLKPLQTGFPDDSDARFVFDRAPALLRR |
| | (SEQ ID NO: 274) | LRLAGGGASTRLVASNHTLFPSPAVDPLPERQDTVVNYGGEEGVEEKALVCRTVYAWREPVSPHLAAEREGMPVEDEEVRWLVDRWLAEE |
| | | DGGGEVWKVLETAGGVASPGPSGTLQCDLYRSSRLPAVLVGDGRLGGISSTLSAYETLLLRGYDVGSVILEDRGLSNDRFLLSYLRKRVPVH |
| | | VLPPIPEDPKDDLTDWFSESSSAFSSLKDSLQSFHSRRVQRLNSMQRKSKYLLWWPFTQHDLVPVDSVTVIDSRFGENFSAYKVKDKTIVPQ |
| | | FDACASWWTQGPDSNLQIELARDMGYAAARYGHVMFPENVHEPALRCAELLLGGVGKDWASRVYFSDNGSTAIEIALKMAFRKYACDHGIIV |
| | | DSEKDIRSEGSVHFKVLALNGSYHGDTLGAMEAQAPSAYTSFLQQPWYSGRGLFLDPPTVYIKNKSANLSLPPSIMHDQLSSCDTCFSSLTEV |
| | | FCKTRDTSSAANVYVSYISQQLSQYAMSNNSEHIAALIIEPVIQGAGGMHLIDPLFQRLLVKECKNRKIPVIFDEVFTGFWRLGVESASELLGCF |
| | | PDISCYAKLMTGGIVPLAATLATEPIFEAFRSDSKLTALLHGHSYTAHPMGCTAAVKAIQWYKDPSTNSNIDLDRMKLKELWDSALVNHLSSLP |
| | | NVKRVVSLGTLCAIELKAEGSDAGYASLYASSLIRQLREEDNIYARPLGNVIYLMCGPCTTQDSCTRQLAKVHRRLQKLN |
| 275 | Glycine max BIO3-BIO1 | MFRFPSVLLSRRLHRRELSSSTTSHPPSLPLSHPIYLIWGSNTGVGKTLVSAGIAAAFLLSSPTPSQFHYLKPLQTGFPSDSDSRFVFNKLRQL |
| | (SEQ ID NO: 275) | SLIRNPHIPLSASHRVINVSPAVTASNPLAGEEGVGPPPELLCKTLYAWEEAVSPHLAAEREGLVVKDSAVLETLGECLEDVAECGAGKERSE |
| | | VLCVVETAGGVASPGPSGSLQCDLYRPFRIPAVLVGDGRLGGISGTISAYESLKLRGYDVVAVVFEDHGLLNEGPLMSYMRNKVPVLVLPPIP |
| | | KDPLNDLMEWFESSHYIFSNLKEIMLSAYFERIKKLHDMPREARDIIWWPFTQHKLVPDGGVTVIDSRCGENFSIFKVQKTEVIAPQFDACASW |
| | | WTQGPDAIMQTELAREMGYAAARFGHVMFPENVHEPALNCAELLLQGVGKGWASRTYFSDNGSTAIEIALKMAFRKFSVDHGLIVHCHEDTT |
| | | NERSTELMVLALQRSYHGDTLGAMEAQAPSSYTGFLQQPWYTGRGLFLDPPSVFMHNSTWNVSIPEGYHLESLKGKSITFASPDEIFHKGRD |
| | | KSELATVYSSYISKVLSGFRGSNNVGALIMEPVIQGAGGMHMVDPLFQRVLVNECRSRKIPVIFDEVFTGFWRLGVETAVELIHCVPDIACFGK |
| | | LLTGGIIPLAVTLATNAVFDSFIGDSKLKALLHGHSYSAHAMGCAAAVKSIQWFKDPCSNPNITSEGRLLRELWDDKMVHRISSHPAIQRVVAL |
| | | GTLCALELKAEGNNAGYGSLYARPLLEKLREDGVYMRPLGNVIYLLCGPCTSPEVCNQLLVKLLRRLEEFDVGKN |
| 276 | Triticum aestivum BIO3- | MLRRLLLRQARRRHCSSSFSSALPLSSPTFAVFGANTGVGKTLVSAGLAASLLRSHAASPSAVLYLKPLQTGFPADSDAGFLYRRVPALVRPS |
| | BIO1 (SEQ ID NO: 276) | HAAHLVASIDTLSPAPSVETPSPSREEAAFSYDGEGKAVRKLLACRTLYAWREAVSPHLAAEREGMTVEDAHVRSLLAACLAEGETLGKGEG |
| | | DVWKLVETAGGVASPGPSGTLQCDLYRHFRLPIILVGDGRLGGISGTLSAYETLLLRGYDVSAVILEDCGLSNDKFLLSYLRNRVPVLVLPPIPE |
| | | DPSDDLTDWFSESSSVFGLLEDALRSFHLKRIQRLNSMQRKSKDLLWWPFTQHNLVPVDCVTVIDSRCGESFSAYKVKDNKLMMIPQFDACA |
| | | SWWTQGPDSKLQIELARDMGYAAARYGHVMFPENAHEPALRCAELLLGGIGKDWASRVYYSDNGSTAIEIALKMAFRKFSLDRGILLDSDKSI |
| | | TNERNIQLKVLALKGSYHGDTLGAMEAQAPSAYTSFLQQPWYSGRGLFLDPPTVFIRNGTCALSLPQSIQNCHLSPGDKCFPSFAEVFCKSR |
| | | DSSAAADLYSTYISQQLSEYSMSSNTEHIAALIIEPVIQGAGGMHMIDPLFQRVLVHECRDRKIPVIFDEVFTGFWRLGVESASELLGCLPDVAC |
| | | YAKLMTGGIVPLAATVTTEAVFEAFKSDSKLTALLHGHSYTAHAMGCSAAVKAIQWFRDPSTNSNLDFDCMKLKELWDGTLVNQLSSLPNVK |
| | | RVVSLGTLCAIELQAEGSDAGYASLYARSLIQQLREEDDIYVRPLGNVIYLMCGPCTTRDVCTEQLSKVYRRISHFNPIH |
| | BioDA/BIO3-BIO1 | |
| | protein sequences- | |
| | MTP removed | |
| 294 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | (SEQ ID NO: 294) | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 295 | Zea mays BioDAΔMTP | MQSPAVEAKAESLHESQEKVVTYGAGAAEQTKVLACCTAYAWREPVSPHLAAEREGMAAGDDEVKGCVEQWLLEEGIGEGGEVWKILETA |
| | protein sequence (SEQ | GGVASPSASGTLQCDLYRPFRLPAILVGDGRLGGISSTLSAYETLLLRGYDVSAVILEDRELSNDKFLLSYLRNRVHVLILPQIPEDPLDDLTDW |
| | ID NO: 295) | FSESSSIFIFLKDELQSFHSRRIERLNSMQRKSKALLWWPFTQHNLVPQDSVTVIDSRYGENFSVYKIKDKMLIPQFDACASWWTQGPDSNLQ |
| | | IELARDMGYAAARYGHVMFPENVHEPALHSAEVLLGGVGKGWASRVYYSDNGSTAIEIALKMAFRKFSLDHGIMASSENSTRNERNIQLKVLA |
| | | LNGSYHGDTLGAMEAQAPSAYTSFLQQPWYSGRGLFLDPPTVYIKSEVYNISLPQSMQHDPQTYGFFSQAEVFCKTRDKTFAADLYSSYIKQ |
| | | KLSEFSLSSSSEHLAALIIEPVIQGAGGMLMIDPLFQRVLVSECRSRKIPVIFDEVFTGFWRLGVESASELLGCLPDIACYAKLMTGGIIPLAATLA |
| | | TEEVFESFESDSKLTALLHGHSYTAHAMGCTAALKAMQWYRGPSTNSNLDTDHMKLKELWDGTLVKQLSSLPNVKRVVSLGTLCAIELKAEG |
| | | ADAGYASLYASSLVQQLRKEDDIYIRPLGNVIYLMCGPCTPRDSCTRQLLKVHRRLCALN |
| 296 | Taxus chinensis | MSRSGQAGEDRTPKQKLHIPSGFEVDLSHPTYVIWGSNTGVGKTLVSTGLAASLLNASQNLLYVKPVQTGFPQDSDSRYVSHKVKDILKQRK |
| | BioDAΔMTP (SEQ ID | PSNNVHISHQVLKASPRAWELLPGSKGFGEVASYERAIQGMRNVCDYEGQTVQGLYKGLADLKLVCKTIYGWYEAVSPHLAAAGEKCSVDD |
| | NO: 296) | CEVVENLQNSLREFNVDENNTNRWSIIETAGGVASPGPSGTLQCDLYRPFRLPSILVGDGRLGGISGTISAYETLCIRGYDICAVILADTGLSNE |
| | | KCLSTYLHDRVPVFVLPKIPDDTNNTLLEWFSESYKVFSEIKDTLQTSYFKRIYRLHEMPMKARQILWWPFTQHSLVTEQSISLIDSRFGENFG |
| | | VYKGKNHKDVKESIVQQFDACASWWTQGPSAILQTELAREVGYTTGRFGHVMYPENVYEPALHCAELLLEGVGKGWATRVFLSDNGSTAIEI |
| | | SLKMAFRKFAFDHGFLPEHFNEDLRKICPQFKVEIGTISYTTCAFFIEMTMEEFSEESYTGRGLFLDPPTVFLHNEEWQLHLPDALMYSDLKYP |
| | | GLVIHASGGMHMIDPLFQRDLVKECRKRRIPVIFDEVFTGFWRLGAESAAEILGCAPDIACYAKLLTGGIVPLAATLATESIFEAFEGKSKLFALL |
| | | HGHSYSGHAIGCAAAIKAIQWFKDAEMNLNICTDGSRLQELWDSKLVAKISALSSVKRVIALGTICALELQADESNTGYGSLLASSLVQKLRDD |
| | | GVYMRPLGNVIYLMCGPATPPAMCSTLLQKLHKRLDEC |
| 297 | Physcomitrium patens | MSSHPSPIEDADEARCVAENVADALDVSYPTYMVWGSNTGVGKTLVSAGLCSAILRLSKCQGNSGAKDLLYLKPVQTGFPSDSDARYVYQE |
| | BioDAΔMTP (SEQ ID | VSRVGRLHPEAISEGLFLCNHTLQVSPAVQISARAGASVPVQPQSAWNAGGLSSSLNGTGMKDFCSYESQLLQGVKSRNIPADGDVYGRAQ |
| | NO: 297) | LRNTLSRFTCKTMWAWYEPISPHLAAAREGSTVPDTMVVEAIQRSLQALSSSRKTSGHREDAGDCAEKWAILETAGGVASPGPSGSLQCDM |
| | | YRSLRFSGLLVGDGRLGGISTTIAAYESLRSRGYDIPAIVIVDSGLSNEEPVLQYLRHKIPVVVLPPLPEDPNKRLEEWFRLAEGTFLELMNVLE |
| | | NSHSKRLRRLEEMPKMAGEILWWPFTQHDLVAQDSITLIDSRSGENFSVYKSGTPSTGKTIATQFDACASWWTQGPDSILQPEIAREVAYTA |
| | | GRYGHVMFPENVHEPALRCAEILLAGAGRGWAERVFFSDNGSTAIEVAIKMAFRKYIVDHGLSDPSTIERRGGQLKVVALKGSYHGDTLGAM |
| | | EAQAPSAYTGFKQQPWHVGRGFFLEPPTVFQRQGRWHLQLPRVFHDSDSSDSASCWKSRDEIFDICRDSTPLANLYIRSINQQLAAAVREE |
| | | NNVPAALIIEPVMHGAGGMDMIDPLYQRMLVRECRRRGIPTIFDEVLSGCWRFGVESAVDLLGCSPDIACYSKLLSGGLVPLAATLATQAVFE |
| | | AFRGISKLDALLHGHSYTAHAVGCASAVVSLQYFSDPSKNSNLLPGGRRLRELWKPELVSLISSHQSVERVVSVGTIFALELRANAAESGYAS |
| | | MLSKELVENLRQEGIYTRPLGNVVYLMCGPLTQPKTCTTMLEKLLALLG |
| 298 | Setaria italica | MSSAAATVPLSTPTFAVFGANTGVGKTLVSAGLVAALLNSRSPSVSAVSYLKPLQTGYPADSDARFVFARAPALLRASSSPRATRLVASCRTL |
| | BioDAΔMTP (SEQ ID | FPSPAVGPEAEPLHQSQEKVVTYGGDGAEEETKVLACRTVYAWREPVSPHLAAEREGMAAGDDEVRGCVDQWLMEEGVGEGGEVWKVL |
| | NO: 298) | ETAGGVASPSASGTLQCDLYRPFRLPAILVGDGRLGGISSTLSAYETLLLRGYDVSTVILEDRDLTNDTFLLSYLRNRVHVLALPQIPEDPSDDL |
| | | TDWFSESSSVFNLLKDSLQSFHSKRVERLNGMQKKSKDLLWWPFTQHNLVPQDSVTVIDSRCGENFSAYKIKDNTMMLVPQFDACASWWT |
| | | QGPDSDLQIELARDMGYAAARYGHVMFPENVHEPALRAAELLLGGVGKGWASRVYYSDNGSTAVEIALKMAFRKFSLDHGILADSEKSIRNE |
| | | RNIQLKVLALNGSYHGDTLGAMEAQAPSAYTSFIQQPWYSGRGLFLDPPTVCIKSETCNVSLPQSMQHYPRTSGDTCFPSLAEVFCKTRDTS |
| | | SAADLYLSYIKQQLSEFSQSSNSDHLAALIIEPVIQGAGGMLMIDPLFQRVLVRECRSRKIPVIFDEVFTGFWRLGVESASELLGCLPDIACYAKL |
| | | MTGGIVPLAATLATEEVFESFKSDSKLTALLHGHSYTAHAMGCMAALKAIQWYRDPSTNSNLDLDHMKLKELWDGALVKQLSSLPNVKRVVS |
| | | LGTLCAIELKAEGSDAGYASLYASSLVQQLRREDDIYIRPLGNVIYLMCGPCTPRDSCSRQLHKVLRRLCDLN* |
| 299 | Phoenix dactylifera | MPPELDLSHPTLVVWGSNTGVGKTIVSAGLAASVLSPSSPNTATFLYLKPVQTGFPRDSDSRFVYRKVSDLFRHRLGGFSAQLLASNHALKA |
| | BioDAΔMTP (SEQ ID | SVPAAREVLGARTERPGEEGEEASTGLCCYEETRVGTGESTTGDFKLVCKTLYGWKEAISPHLAVEREGMAVEDSSLREVLGRLLNSSMGG |
| | NO: 299) | GGDRRKENVWRVIETAGGVASPGPSGSIQCDLYRPFRLPAVLVGDGHLGGISTTLSAYESLTLRGYDVIAIILADHGLSNEIPLLSNLRHRVPVL |
| | | VLPPVPEDPLNDLVDWFCESSKAFSSLQDLVQSTHSKRIQRLHDMPRKAGNILWWPFTQHKLVPEEIVTVIDSRCGENFAVHKVRNDREMIV |
| | | RQFDACASWWTQGPDFALQIELARDMGYSAARYGHVMFPENVYEPALHCAELLLEGVGKGWASRTYYSDNGSTAIEIALKMAFRKFLLDNE |
| | | MLMDSGNGISSERCIELRVLALNGSYHGDTLGAMEAQAPSSYTGFLQQPWYSGRGLFLDPPTIFIHKEIWNLSVPESFESDQLKPEDMRFSS |
| | | SDALFCPSRDSSAIAGIYSAYISNQLLQFSKLSCSTQIGALIIEPVIQAAGGMHMIDPLFQRILVRECRNRRIPVVFDEVFTGFWRLGKESAAELL |
| | | GCVPDVACFGKLMSGGIIPLAVTLTTEAVFEAFKGDSKLMALLHGHSYSAHAMGCTAAAKSIQWFRDPFTNPNVEFKEKKLKELWEMKLVLQI |
| | | SSLPAVQRVITLGTLCAVELKAEGSDAGYASLYASLLVQQLREDGLYVRPLGNVIYLMCGPCTSSHFCSQQLQQVYQRLHEFSQHQLRDSQS |
| | | FQREG |
| 300 | Ostreococcus tauri | MLGSASDGAASGNGRALELSIASFAIWGANTGVGKTLASAGLARAATRRGTATTFLKPVQTGYPEDSDAGFVARQTKGNETAGEHAAVASG |
| | ATCC 22665 | STEGGGEGFAAGVTRAHTEFAWRRAAGPHLTAAEEGRPVSDEEIVRATRNYLREFAESAREGNARGFAVIETAGGVASPGPSGTLQCEILRA |
| | BioDAΔMTP (SEQ ID | LRLPAILIGDGRLGGISTTTSAYESLHARGYDVVGVLMADDGYANHEAVAKILPSGTKVFALPPLPPQGKADAWLEKSAGVFSEAFETAMNWH |
| | NO: 300) | EKRIQALQKLPGEALEKIWWPFTQHATVEPDTVTVIDGRYGEDFEIYDQEKGRDGAIDLRFDGAASWWTQGVSAELQQELNSAAANAAGRY |
| | | GHVMFPENVHQPSMDATNALLEGPGKGWGSRVFYSDNGSTATEVGLKMAFRKYYVDAGLVSKDGHQRAKDLINDGSCHLPQLRIIALDGSY |
| | | HGDTLGAMDMQSPSVFTGPMQTPWYQPRGLFLQPPSLAIRNGEWTVALPPHGLLNDGEDRLFHDGKDLESNTTWKSKADAFDIGKRLDSP |
| | | LANEYRQAVDKALERAEQDAKAGHMGPVGALMMEPVLHGAGGMVLIDPLFQNILMKVCRERKIPIVLDEVFAGIWRLGTEGAWEIIGEKPDIS |
| | | CYAKLLTGGLMPMSATVATADVFDVFYGPGKAQGLLHGHSYTAYPIGCAVASKALSLYKDPAKNPNLIVDASSKGDTPTAYLRELWDESKLK |
| | | EISNKSSVRRVFGQGCVLAVELEVEGEGGYSSNAAKEVVLRLRPHSVQARPLGNVVYLMCAPTTPKATCDRLLNVLAEELSE |
| 301 | Helianthus annuus | MLSTLSHTLSHPIYTIWSANTGLGKTLVSAGIAAASVASPPSKLVYIKPIQTGYPLDSDAAFVYRKIPQILHRRNPNLTLTASNQTLNVSIPAAKVK |
| | BioDAΔMTP (SEQ ID | IGDNFSDSRNGFGEFGFCEEMKVGEGVGEGSELICKTIYAWNEAISPHLASEREGALVGDEEVLVLLKKCLEIGVGSEGQGSVLSLVETAGGV |
| | NO: 301) | ASPGSSGSLQCDLYRPFRLPSILVGDGKLGGISGTISAYESLTIRGYDVVAIVLEDHGLENEVPLLSYLRNRVPVLVLPSIPKDPLDNLMEWYDE |
| | | SQSVFDSLKGIMLSSYEKQMYRLHEMPKKAQEIFWWPFTQHKLVPQEKVTVIDSRCGENFAVHKVKDHDYITQMFDACASWWTQGPNAAL |
| | | QIKLAREMGYTAARYGHVMFPENVYEPALQCAELLLQGVGKGWASRVFFSDNGSTAIEIALKMAFRKFLADNKLLMDLPHYNTDESNIELKVV |
| | | ALNGSYHGDTLGAMDAQAPSPYTGFLQQPWYTGRGVFLDPPFVSMCDGVWKIAVPKKMHYEDVILEDTSFNSRDQVFNRSRDDSKLASIYS |
| | | TYISQELLPSSGSTKCSYAGALIIEPVIQGAGGMLMVDPLFQRILVKECKNRKIPVIFDEVFTGYWRLGVESAAELLFCQPDIACFAKLMTGGMI |
| | | PLAATLATNDVFESFVGDSKLNALLHGHSYSAHAVGCATACNSIKWFKDSQTNPNLVPGQNLLRELWDAELVRKISFHPAVQRVVSLGTLFAL |
| | | ELKVEDNDAGYASLSGTSLLLKLREDGIYMRPLGNVIYMMCGPCTSHRVCLQMLEKLYMRLDEFKQEKIGTARDYDHDHRLPIA |
| 302 | Quercus robur | MSTTAHSQLHVPLNHPTFLVWASNTSLGKTLVSTGLAASSLLNPTPKPRKFLYLKPIQTGFPSESDSSSVFHKLSRLSLLRRHNLPHFSLFSSN |
| | BioDAΔMTP protein | HILKASFPAANSALGQEISETPHSGMRDLGYYEEAKPEGDVGGGAASQLVCKTLYAWREAVSPHLAAERESGVVEDAAVLELLQKCLRFELE |
| | (SEQ ID NO: 302) | GGGEKGKETEIFCVVETAGGVASPGPSGSLQCDLYRPFRLPGILVGDGRLGGISGTISAYESLKLRGYDIAAVVFEDHGLVNEMPLLSYLRNR |
| | | VPVLVLPSVPKDLSNDLMEWFDESRGVFDSLKEEMLSAYSKRMMRLQDMPKKAGEIFWWPFTQHQLVPEEAVTVIDSRHGENFAVFKAQN |
| | | NEFITQQFDACASWWTQGPDTTLQTELARDMGYAAARFGHVMFPENVYEPALECAELLLEGVGKGWASRTFFSDNGSTAIEIALKMAFRKF |
| | | SYDHGILSDGRKDNTASQRVKLMVLALQGSYHGDTLGAMEAQAPSSYTGFLQQPWYTGRGLFLDPPTVYMCNGVWKLSLPEGLHLEIPKLE |
| | | NKGFSSRDEIFHKIRDKSDLARIYSSYISEQLSQYSGSGGFYHIGALIMEPVILGAGGMHMIDPLFQRVLVNECRSKKIPVIFDEVFTGFWRLGR |
| | | ETAAELLGCVPDIACFAKLMTGGIIPLAATLATNAIFNSFIGESKLKALLHGHSYSGHAMGCTAAVKSIKWFKDPQTNLNITDEGRSLRELWDED |
| | | VVQQISSHPAVERVVALGTLFALELRAEGSNAGYASQYASSLLQNLRQDGIYTRPLGNVIYLMCGPCTSPEICSQLLVKLYKRLEDFDQFHQIR |
| | | EENSESYGG |
| 303 | Thraustotheca clavata | MSLDQSVPNAVPTSLHAPIVQVFGSNTDVGKTVIAAGLCRAALLSSKNATVGYIKPLQTGGDAMMDAEFLNRHAGNADNRLTSHTLFSWETA |
| | BioDAΔMTP (SEQ ID | VSPHLAASLENKAVDDATVVAKLSKTLKQIQKEEKGSALTVVETAGGVCSPSASGKFQCDVYRPLRLPTVLVGDGCLGGISATMSALDSLLIR |
| | NO: 303) | GYDVSCIVLIEQDNLSNGEAISERASELGIPIFSLDKLPPQPEPLDLWYENSTETFRSVHDTIASTHKARVARLQELPSIAKKALWWPFTQHKSN |
| | | KGVLVIDSAYGDTFATYNQKNQAIEPMYDACASWWTQGVGHGNAKMALSLATAAGRYGHVMFPENAHEPAIALSEMLLKSVGKNWAGKVF |
| | | FSDDGSTAVEIGIKMALRKYAVDHGLGMSFEGINDVLVLAQEGCYHGDTLGTMNIAEPSVYNKTQHPWYKPKALFVHPPSVKVASNGQMQV |
| | | TWQNVNPTLVTHLPTLDDLFDISRDTSPLTQQYTEYVESLLDSVPSTTVVGALIVEPILIGAGGMVFVDPLFQRTMVRVCQQRKIPVIFDEVFSG |
| | | FWRLGAESARDLLQVNPDIACYAKLLTGGAVPMSTTLATNEVFDAFYTDSKADALLHGHSFTANPVGCAAAVTALDMYKTIEKKNSTRVHWN |
| | | VDQVAALAARPGIARAFQLGTVLVIELESTSRGYTDTTGQDVLVRLRNHGIYARSLGNVLYIMASPLSTPATCIKIFNTVVDNLFA |
| 304 | Gibberella zeae | MCNAASKKGDRTWFLKPVSTGAVDEADGCHIQRFASSTNHETLFQYDIPCSPHIAAKVSGKPIPSDEAVLAKIHDSVSRYASEGPGWLFLETA |
| | BioDAΔMTP (SEQ ID | GGVHSPGPSGTPQADLYTPLRAPVILVGDSKLGGISQTISAYESLRMRGHDIESILLFQDMKYENYQYLKDYFSKQGGIPVDTVPEPPSRLPDI |
| | NO: 304) | QQDTEQMREYYASQTSNVSHVLDHLDKRGKQRISRLESLSEKASKSIWYPFTQQKLVTADTISAIDSAHGDYFQVLNKTYENLLQPAFDGSAS |
| | | WWSQGLGHANSRLTLAAAYAAGRYGHVMFAEAIHEPALALAEMMLEGAQNSRFSRVFYSDNGSTGCEVAVKMALRAARLRYGWGPNDNL |
| | | QILGLKGSYHGDTIGAMDCAEPCVYNEKIEWYEGKGYWFDYPTIQCVEGKWVVSAPNGMGQDQKLESISEVFNLESRLKTEQYRGYEEYIEK |
| | | TLKKLQDQGRKFGALMMEPIILGAGGMIFVDPLFQRALVDVVRRSPHLFGTKDALPAQGPQWSGLPVIFDEVFTGLYRLGRFTAASFLGTEAD |
| | | ISVNAKLLTGGLVPLCTTMASESIFDAFKSDDKSDALLHGHSYTAHAVGCQVAVESVSEMQSMEKNGEWEWAEKDWAEDETQAWSVWSRD |
| | | FVTDMSHNPQVLGVWALGSVLAISLRDDDGVGYKSLAAKKIQSHLRQGTGSWNAHSRVLGNVFYVMASQKTSRQSIRELQGLLVDALSK |
| 305 | Hordeum vulgare BIO3- | MSSSFSSALPLSSPTFAVFGANTGVGKTLVSAGLAASLLRSHAASPSAVLYLKPLQTGFPDDSDAGFLYRRVPALLRPSHAAHLAASIDTLSPA |
| | BIO1ΔMTP (SEQ ID | PSVETQSPSREEAAFSYDGEGEAVRKLLACRTLYAWREAVSPHLAAEREGMAVEDAHVRSLLAACLVEGENLGKGEGDVWKLVETAGGVA |
| | NO: 305) | SPGPSGTLQCDLYRQFRLPTILVGDGRLGGISCTLSAYETLLLRGYDVSAVILEDHGLSNNKFLLSYLRNRVPVLVLPPIPEDPSDDLTDWFSE |
| | | SSSVFGLLEDALRSFHLTRIQRLNSMQRKSKDLLWWPFTQHNLVPVDCVTVIDSRCGESFSAYKVKDNKMMMIPQFDACASWWTQGPDSKL |
| | | QIELARDMGYAAARYGHVMFPENAHEPALRCAELLLGGIGKDWASRVYYSDNGSTAIEIALKMAFRKFSLDRGILLDSDKSIITNGRNIQLKVLA |
| | | LKGSYHGDTLGAMEAQAPSAYTSFLQQPWYSGRGLFLDPPTVFIKNGTCTLSLPQAIQNCHLSPGDKCFPSLAEVFCKTRDSSSAADLYSTYI |
| | | SQQLSEYSLSSNTEHIAALIIEPVIQGAGGMHMIDPLFQRVLVHECRDRKIPVIFDEVFTGFWRLGVESASELLGCLPDVACYAKLMTGGLVPLA |
| | | ATVTTEAVFEAFKSDSKLTALLHGHSYTAHAMGCSAAVKAIQWFRDPSTNSNLDFDCMKLKELWDGTLVNQLSSLPNVKRIVSLGTLCAIELQ |
| | | AEGSDAGYASLYARSLIQQLREEDDIYVRPLGNVIYLMCGPCTTRDVCTEQLSKVYRRISHFNPVHRG |
| 306 | Brassica napus BIO3- | MSTAVPSHFNLPLNHPTYLIWSANTSLGKTLVSTGIASSFLLHQPSSSPPAAHSAKLLYLKPIQTGFPSDSDSRFVFSKLDSLSLRRRIPLSVSN |
| | BIO1ΔMTP (SEQ ID NO: | SVLRSSLPVAESMRRNIKVSESGMCDLNFREEKTVTGAPELLCKTLYAWEAAISPHLAAEREHATVEDSVVLKMVEQEMECGSKANVLCLVE |
| | 306) | TAGGVASPGPSGTLQCDLYRPFRLPGILVGDGRLGGISGTIAAYESLKLRGYDVAAVVFEDHGLVNEVPLTSYLRNKVPVLVLPPVPKDPSDD |
| | | LIEWFVESDGVFKALKEVMVSVYLERVERLNGMAKQAGEVFWWPFTQHKLVPEDNVTIIDSRCGENFSVFKASDNNSITQQFDACASWWTQ |
| | | GPDPAFQAELAREMGYTAARFGHVMFPENVYEPALKCAELLLDGVGKGWASRVYFSDNGSTAIEIGLKMAFRKFCVDHETLFGLSEDAEEKK |
| | | HVDVKVLALRGSYHGDTLGAMEAQAPSPYTGFLQQPWYTGRGLFLEPPTVFLSNGTWNLSLPESFSQTASEECGTFTTRDEIFDKSRDTSVL |
| | | ATVYSTYVSEQLQEYSGISQSAHVGALIIEPVIHGAGGMHMVDPLFQRVLVNECRNRKIPVIFDEVFTGFWRLGVETTADLLGCKPDIACFAKL |
| | | LTGGMIPLAVTLATDAVFDSFSGDSKLKALLHGHSYSAHAMGCATATKAIEWFKDPETNHNIDSQRGTLRELWDEELVQHISCHCAVERVVVL |
| | | GTLFALELKVDASNSGYASLYAKSLLEMLREDGIFMRPLGNVVYLMCGPCTSPEICRRLLTKLHKRLGEFNRA |
| 307 | Gossypium hirsutum | MSTLTIPLSHPTYIIWSSNTSLGKTLVSTGLSSSFLLSPSSSSKKFLYLKPLQTGFPSDSDSRFLFQKLSSLSLSRNLPLFSSHSVLLSSLPAAKS |
| | BIO3-BIO1ΔMTP (SEQ | FKPNEFSLNESREMCELGFYEEKKVLEAGRVAPELVSETIYAWEGALSPHFAAEREGGAVEDSELVKRLETRLKEGLLEGGVERGKLDGFCV |
| | ID NO: 307) | VETAGGVASPGPSGTLQCDLYRPLRFPGVLVGDGRLGGISGTISAYESLKLRGYDVVAIVIEDHGLINEIPLKSYLRSRVPVFVLPPIPQDPSND |
| | | LMEWFDESCNVFNSLKDIMLEAYLERMRRLNEMPRKAGDVFWWPFTQHKLVPPSTVTVIDSRCGENFSVYKVQNKEYITQQFDACASWWT |
| | | QGPDATLQTELARDMGYAAARFGHVMFPENVYEPALECAELLLDGVGKGWATRVYFSDNGSTAIEIALKMAFRKFSSDHGILPELLKNNPTE |
| | | SCTELMVLALKGSYHGDTLGAMEAQAPSSFTGFLQQPWYTGRGLFLDPPTVFMHNGKWTVSLPEVFHSATLKPEDTTFRSRDEIFLKSRDE |
| | | SNLAGLYSLYVSQQLSQYSTLSETNKQIGALIMEPIIQGAGGMHMVDPLFQRMLVNECRHCRIPVIFDEVFTGFWRLGVESAAELLGCVPDIAC |
| | | FAKLMTGGIIPLAATMATDAIFDSFTGDSKLKALLHGHSYSAHAMGCTAAAKSIKWFKDPNTNLNITSEKSQKMLLRELWDAELVQQFSSHPSI |
| | | SRVVTLGTLFALELQADESDAGYASLYARSLVQMLREDCIYTRPLGNVIYVMCGPCTSPKMCTLQLLKLYTKLEEFTLVKTKMNII |
| 308 | Oryza sativa | MSTSSSSSAAAAAVPLTSPAFAVFGANTGVGKTLVSAGLVASLLASPSPSPSTVAYLKPLQTGFPDDSDARFVFDRAPALLRRLRLAGGGAST |
| | BIO3-BIO1ΔMTP | RLVASNHTLFPSPAVDPLPERQDTVVNYGGEEGVEEKALVCRTVYAWREPVSPHLAAEREGMPVEDEEVRWLVDRWLAEEDGGGEVWKV |
| | (SEQ ID NO: 308) | LETAGGVASPGPSGTLQCDLYRSSRLPAVLVGDGRLGGISSTLSAYETLLLRGYDVGSVILEDRGLSNDRFLLSYLRKRVPVHVLPPIPEDPKD |
| | | DLTDWFSESSSAFSSLKDSLQSFHSRRVQRLNSMQRKSKYLLWWPFTQHDLVPVDSVTVIDSRFGENFSAYKVKDKTIVPQFDACASWWTQ |
| | | GPDSNLQIELARDMGYAAARYGHVMFPENVHEPALRCAELLLGGVGKDWASRVYFSDNGSTAIEIALKMAFRKYACDHGIIVDSEKDIRSEGS |
| | | VHFKVLALNGSYHGDTLGAMEAQAPSAYTSFLQQPWYSGRGLFLDPPTVYIKNKSANLSLPPSIMHDQLSSCDTCFSSLTEVFCKTRDTSSA |
| | | ANVYVSYISQQLSQYAMSNNSEHIAALIIEPVIQGAGGMHLIDPLFQRLLVKECKNRKIPVIFDEVFTGFWRLGVESASELLGCFPDISCYAKLMT |
| | | GGIVPLAATLATEPIFEAFRSDSKLTALLHGHSYTAHPMGCTAAVKAIQWYKDPSTNSNIDLDRMKLKELWDSALVNHLSSLPNVKRVVSLGTL |
| | | CAIELKAEGSDAGYASLYASSLIRQLREEDNIYARPLGNVIYLMCGPCTTQDSCTRQLAKVHRRLQKLN |
| 309 | Glycine max BIO3- | MSSSTTSHPPSLPLSHPIYLIWGSNTGVGKTLVSAGIAAAFLLSSPTPSQFHYLKPLQTGFPSDSDSRFVFNKLRQLSLIRNPHIPLSASHRVIN |
| | BIO1ΔMTP (SEQ ID NO: | VSPAVTASNPLAGEEGVGPPPELLCKTLYAWEEAVSPHLAAEREGLVVKDSAVLETLGECLEDVAECGAGKERSEVLCVVETAGGVASPGP |
| | 309) | SGSLQCDLYRPFRIPAVLVGDGRLGGISGTISAYESLKLRGYDVVAVVFEDHGLLNEGPLMSYMRNKVPVLVLPPIPKDPLNDLMEWFESSHY |
| | | IFSNLKEIMLSAYFERIKKLHDMPREARDIIWWPFTQHKLVPDGGVTVIDSRCGENFSIFKVQKTEVIAPQFDACASWWTQGPDAIMQTELARE |
| | | MGYAAARFGHVMFPENVHEPALNCAELLLQGVGKGWASRTYFSDNGSTAIEIALKMAFRKFSVDHGLIVHCHEDTTNERSTELMVLALQRSY |
| | | HGDTLGAMEAQAPSSYTGFLQQPWYTGRGLFLDPPSVFMHNSTWNVSIPEGYHLESLKGKSITFASPDEIFHKGRDKSELATVYSSYISKVLS |
| | | GFRGSNNVGALIMEPVIQGAGGMHMVDPLFQRVLVNECRSRKIPVIFDEVFTGFWRLGVETAVELIHCVPDIACFGKLLTGGIIPLAVTLATNAV |
| | | FDSFIGDSKLKALLHGHSYSAHAMGCAAAVKSIQWFKDPCSNPNITSEGRLLRELWDDKMVHRISSHPAIQRVVALGTLCALELKAEGNNAGY |
| | | GSLYARPLLEKLREDGVYMRPLGNVIYLLCGPCTSPEVCNQLLVKLLRRLEEFDVGKN |
| 310 | Triticum aestivum BIO3- | MLSSPTFAVFGANTGVGKTLVSAGLAASLLRSHAASPSAVLYLKPLQTGFPADSDAGFLYRRVPALVRPSHAAHLVASIDTLSPAPSVETPSP |
| | BIO1ΔMTP (SEQ ID NO: | SREEAAFSYDGEGKAVRKLLACRTLYAWREAVSPHLAAEREGMTVEDAHVRSLLAACLAEGETLGKGEGDVWKLVETAGGVASPGPSGTL |
| | 310) | QCDLYRHFRLPIILVGDGRLGGISGTLSAYETLLLRGYDVSAVILEDCGLSNDKFLLSYLRNRVPVLVLPPIPEDPSDDLTDWFSESSSVFGLLE |
| | | DALRSFHLKRIQRLNSMQRKSKDLLWWPFTQHNLVPVDCVTVIDSRCGESFSAYKVKDNKLMMIPQFDACASWWTQGPDSKLQIELARDMG |
| | | YAAARYGHVMFPENAHEPALRCAELLLGGIGKDWASRVYYSDNGSTAIEIALKMAFRKFSLDRGILLDSDKSITNERNIQLKVLALKGSYHGDT |
| | | LGAMEAQAPSAYTSFLQQPWYSGRGLFLDPPTVFIRNGTCALSLPQSIQNCHLSPGDKCFPSFAEVFCKSRDSSAAADLYSTYISQQLSEYS |
| | | MSSNTEHIAALIIEPVIQGAGGMHMIDPLFQRVLVHECRDRKIPVIFDEVFTGFWRLGVESASELLGCLPDVACYAKLMTGGIVPLAATVTTEAV |
| | | FEAFKSDSKLTALLHGHSYTAHAMGCSAAVKAIQWFRDPSTNSNLDFDCMKLKELWDGTLVNQLSSLPNVKRVVSLGTLCAIELQAEGSDAG |
| | | YASLYARSLIQQLREEDDIYVRPLGNVIYLMCGPCTTRDVCTEQLSKVYRRISHFNPIH |
| | MODIFIED BIODA/BIO3- | |
| | BIO1 PROTEIN | |
| | SEQUENCES | |
| 15 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A507K | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 15) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQKPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 16 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A507S | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 16) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQSPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 17 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 17) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGCGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 18 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609F | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 18) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGFGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 19 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609H | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 19) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGHGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 20 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609I | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 20) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGIGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 21 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609M | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 21) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGMGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 22 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609N | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 22) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGNGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 23 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609T | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 23) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGTGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 24 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609V | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 24) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGVGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 25 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609W | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 25) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGWGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 26 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein C388D | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 26) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDADASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 27 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein C388T | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 27) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDATASWWTQGPDPTFQAELAREMGYTAARFGHVMFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 28 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 28) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPCTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 29 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348D | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 29) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPDTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 30 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348N | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 30) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPNTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 31 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348S | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 31) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPSTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 32 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348T | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 32) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPTTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 33 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348V | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 33) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPVTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 34 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | G513DEL | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | (SEQ ID NO: 34) | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 35 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein G513L | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 35) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTLFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 36 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein G513P | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 36) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTPFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 37 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein G608E | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 37) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHEAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 38 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein G700A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 38) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHAHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 39 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein G700S | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 39) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHSHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 40 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein L786S | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 40) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLSIMLREDGIFTRPLGNVIYLMCGPCTSPEICRRLLTKLYKRLGEFNRT |
| 41 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P421L | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 41) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFLENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 42 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P508DEL | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 42) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQASPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 43 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P508L | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 43) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQALSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 44 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P508T | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 44) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQATSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 45 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P510L | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 45) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSLYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 46 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q506A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 46) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAAAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 47 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q516C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 47) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLCQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 48 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q517D | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 48) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQDPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 49 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q517F | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 49) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQFPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 50 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q517M | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 50) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQMPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 51 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q517T | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 51) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQTPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 52 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q517Y | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 52) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQYPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 53 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein R756S | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 53) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQSVVVIGTLFALELKADASNSGYASLYAKSLLIMLREDGIFTRPLGNVIYLMCGPCTSPEICRRLLTKLYKRLGEFNRT |
| 54 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 54) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPCPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 55 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509D | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 55) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPDPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 56 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509E | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 56) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPEPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 57 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509G | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 57) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPGPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 58 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509V | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 58) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPVPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 59 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509W | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 59) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPWPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 60 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S704H | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 60) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYHAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 61 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein T512C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 61) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYCGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 62 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein T512D | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 62) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYDGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 63 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein T512Q | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 63) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYQGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 64 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein T512R | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 64) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYRGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 65 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein T512V | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 65) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYVGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 66 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein W391L | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 66) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASLWTQGPDPTFQAELAREMGYTAARFGHVMFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 67 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein W391M | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 67) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASMWTQGPDPTFQAELAREMGYTAARFGHVMFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 68 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein W392A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 68) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWATQGPDPTFQAELAREMGYTAARFGHVMFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 69 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein W392G | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 69) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWGTQGPDPTFQAELAREMGYTAARFGHVMFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 70 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein W392S | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 70) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWSTQGPDPTFQAELAREMGYTAARFGHVMFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 71 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511D | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 71) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPDTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 72 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511E | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 72) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPETGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 73 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511F | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 73) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPFTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 74 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511M | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 74) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPMTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 75 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511P | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 75) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPPTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 76 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511R | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 76) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPRTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 77 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511V | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 77) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPVTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 78 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511W | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 78) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPWTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 79 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A389F | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 79) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACFSWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 80 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609K | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 80) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGKGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 81 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609R | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 81) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGRGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 82 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein A609Y | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 82) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGYGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 83 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein C388M | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 83) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDAMASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 84 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 84) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPATQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 85 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348E | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 85) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPETQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 86 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348I | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 86) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPITQHKLVHQETVTVIDSRCGENFSIYKASD |
| | | NSSLSQQFDACASWWTQGPDPTFQAELAREMGYTAARFGHVMFPENVYEPALKCAELLLDGVGKGWASRVYFSDNGSTAIEIALKMAFRKF |
| | | CVDHNFCEATEEEKHIVVKVIALRGSYHGDTLGAMEAQAPSPYTGFLQQPWYTGRGLFLDPPTVFLSNGSWNISLPESFSEIAPEYGTFTSRD |
| | | EIFDKSRDASTLARIYSAYLSKHLQEHSGVRQSAHVGALIIEPVIHGAGGMHMVDPLFQRVLVNECRNRKIPVIFDEVFTGFWRLGVETTTELL |
| | | GCKPDIACFAKLLTGGMVPLAVTLATDAVFDSFSGDSKLKALLHGHSYSAHAMGCATAAKAIQWFKDPETNHNITSQGKTLRELWDEELVQQI |
| | | SSHSAVQRVVVIGTLFALELKADASNSGYASLYAKSLLIMLREDGIFTRPLGNVIYLMCGPCTSPEICRRLLTKLYKRLGEFNRT |
| 87 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348K | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 87) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPKTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 88 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348M | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 88) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPMTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 89 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F348Q | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 89) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPQTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 90 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F370L | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 90) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPLTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 91 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein F420I | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 91) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPITQHKLVHQETVTVIDSRCGENFSIYKASD |
| | | NSSLSQQFDACASWWTQGPDPTFQAELAREMGYTAARFGHVMFPENVYEPALKCAELLLDGVGKGWASRVYFSDNGSTAIEIALKMAFRKF |
| | | CVDHNFCEATEEEKHIVVKVIALRGSYHGDTLGAMEAQAPSPYTGFLQQPWYTGRGLFLDPPTVFLSNGSWNISLPESFSEIAPEYGTFTSRD |
| | | EIFDKSRDASTLARIYSAYLSKHLQEHSGVRQSAHVGALIIEPVIHGAGGMHMVDPLFQRVLVNECRNRKIPVIFDEVFTGFWRLGVETTTELL |
| | | GCKPDIACFAKLLTGGMVPLAVTLATDAVFDSFSGDSKLKALLHGHSYSAHAMGCATAAKAIQWFKDPETNHNITSQGKTLRELWDEELVQQI |
| | | SSHSAVQRVVVIGTLFALELKADASNSGYASLYAKSLLIMLREDGIFTRPLGNVIYLMCGPCTSPEICRRLLTKLYKRLGEFNRT |
| 92 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein G513A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 92) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTAFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 93 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein G608A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 93) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHAAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 94 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein G608I | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 94) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHIAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 95 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein G610H | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 95) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAHGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 96 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein G700C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 96) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHCHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 97 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein H496S | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 97) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYSGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 98 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein M419I | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 98) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVIFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 99 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein M612L | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 99) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGLHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 100 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P347A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 100) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWAFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 101 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P347E | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 101) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWEFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 102 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P421A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 102) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFAENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 103 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P421E | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 103) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFEENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRTNRT |
| 105 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P421W | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 105) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFWENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 106 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P510A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 106) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSAYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 107 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P510C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 107) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSCYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 108 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P510E | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 108) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSEYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 109 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P510Q | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 109) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSQYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 110 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P510V | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 110) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSVYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 111 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P529A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 111) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDAPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 112 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q350H | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 112) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTHHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 113 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q350S | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 113) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTSHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 114 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q517H | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 114) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQHPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 115 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q517I | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 115) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQIPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 116 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Q517W | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 116) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQWPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 117 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein R756K | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 117) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQKVVVIGTLFALELKADASNSGYASLYAKSLLIMLREDGIFTRPLGNVIYLMCGPCTSPEICRRLLTKLYKRLGEFNRT |
| 118 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein R790C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 118) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLCEDGIFTRPLGNVIYLMCGPCTSPEICRRLLTKLYKRLGEFNRT |
| 119 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein R797Q | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 119) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTQPLGNVIYLMCGPCTSPEICRRLLTKLYKRLGEFNRT |
| 120 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S390C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 120) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACACWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 121 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S494A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 121) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGAYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 122 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 122) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPAPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 123 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509F | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 123) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPFPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 124 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509H | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 124) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPHPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 125 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509I | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 125) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPIPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 126 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509K | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 126) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPKPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 127 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509L | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 127) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPLPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 128 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509M | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 128) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPMPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 129 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509N | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 129) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPNPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 130 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509Q | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 130) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPQPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 131 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509R | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 131) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPRPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 132 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509T | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 132) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPTPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 133 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S509Y | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 133) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPYPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 134 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein S704P | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 134) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYPAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 135 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein T393V | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 135) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWVQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 136 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein T512G | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 136) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYGGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 137 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein T512I | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 137) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYIGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 138 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein T512N | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 138) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYNGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 139 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein T512W | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 139) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYWGFLQQPWYTGRGLFLDPPTVFLSNGSWNISLPESFSEIAPEYGTFT |
| | | SRDEIFDKSRDASTLARIYSAYLSKHLQEHSGVRQSAHVGALIIEPVIHGAGGMHMVDPLFQRVLVNECRNRKIPVIFDEVFTGFWRLGVETTT |
| | | ELLGCKPDIACFAKLLTGGMVPLAVTLATDAVFDSFSGDSKLKALLHGHSYSAHAMGCATAAKAIQWFKDPETNHNITSQGKTLRELWDEELV |
| | | QQISSHSAVQRVVVIGTLFALELKADASNSGYASLYAKSLLIMLREDGIFTRPLGNVIYLMCGPCTSPEICRRLLTKLYKRLGEFNRT |
| 140 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein V354A | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 140) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLAHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 141 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein V354E | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 141) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLEHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 142 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein V354L | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 142) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLLHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 143 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein V354N | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 143) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLNHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 144 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein V354T | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 144) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLTHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 145 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein W391F | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 145) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASFWTQGPDPTFQAELAREMGYTAARFGHVMFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 146 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein W392C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 146) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWCTQGPDPTFQAELAREMGYTAARFGHVMFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 147 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein W392D | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 147) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWDTQGPDPTFQAELAREMGYTAARFGHVMFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 148 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein W392M | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 148) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWMTQGPDPTFQAELAREMGYTAARFGHVMFPENVYEPALKCAELLLDGVGKGWASRVYFSDNGSTAIEIALKMAFRK |
| | | FCVDHNFCEATEEEKHIVVKVIALRGSYHGDTLGAMEAQAPSPYTGFLQQPWYTGRGLFLDPPTVFLSNGSWNISLPESFSEIAPEYGTFTSR |
| | | DEIFDKSRDASTLARIYSAYLSKHLQEHSGVRQSAHVGALIIEPVIHGAGGMHMVDPLFQRVLVNECRNRKIPVIFDEVFTGFWRLGVETTTEL |
| | | LGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 149 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511C | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 149) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPCTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 150 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511H | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 150) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPHTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 151 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511I | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 151) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPITGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 152 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511K | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 152) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPKTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 153 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y511Q | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 153) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPQTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 154 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y520N | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 154) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWNTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 155 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein Y520W | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 155) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFPENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWWTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| | BIODA/BIO3-BIO1 | |
| | NUCLEIC ACID | |
| | SEQUENCES | |
| 156 | Arabidopsis thaliana | ATGATTCCCGTGACCGCCACTCTTATTCGTCACAGGTTGAGGCACTTGCGTCATCGAATTAGGTTTAAATCAACCTCTGTATCACCATTC |
| | BioDA nucleic acid | CATTTGCCGCTCAATCATCCTACTTATTTGATTTGGTCTGCCAACACTAGCCTGGGCAAAACTTTGGTCAGTACGGGGATAGCAGCCAG |
| | (SEQ ID NO: 156) | TTTTCTCTTACAACAACCTAGTTCATCAGCAACAAAACTATTGTATCTCAAACCCATCCAGACAGGTTTTCCTAGTGATTCTGACTCAAGA |
| | | TTTGTTTTTTCAAAGCTCGACTCATTGTCATTGCGAAGACAAATTCCTATAAGTATCTCAAATTCCGTACTTCATTCATCACTCCCTGCTG |
| | | CTAAGTCTCTTGGCCTAAATGTTGAAGTAAGCGAGTCTGGAATGTGTAGTCTCAATTTTCGGGATGAGAAAACTGTTACCGGCGCTCCA |
| | | GAGTTGTTGTGCAAGACACTTTATGCTTGGGAGGCTGCAATTTCACCCCATCTTGCTGCAGAGAGAGAAAACGCAACTGTTGAAGACTC |
| | | TGTGGTTCTTCAAATGATTGAGAAGTGCCTGAAGGAGGAGATGGAGTGCGGAGTTAAATCTGAGAAGAGTGATCTCTTGTGTCTAGTCG |
| | | AAACTGCTGGCGGAGTTGCTTCTCCTGGTCCATCTGGAACCTTGCAATGTGACCTGTATAGGCCGTTTAGGCTCCCCGGAATTCTAGTT |
| | | GGAGACGGCAGGTTGGGGGGTATCTCTGGCACAATAGCAGCCTATGAGAGTCTCAAGCTTCGTGGATACGATATCGCTGCTGTTGTCT |
| | | TCGAAGATCACGGACTAGTTAACGAAGTGCCACTTACATCATATTTGAGGAATAAGGTACCTGTCCTCGTATTACCTCCTGTCCCTAAAG |
| | | ACCCTTCAGACGACTTGATCGAATGGTTCGTGGAGTCCGATGGCGTATTCAAAGCACTTAAGGAGACGATGGTATTAGCGAACCTTGAA |
| | | CGTCTTGAAAGGTTGAATGGTATGGCCAAACTGGCTGGTGAAGTGTTTTGGTGGCCATTCACACAGCATAAGCTTGTTCACCAAGAGAC |
| | | CGTAACTGTGATTGACTCCAGATGTGGCGAGAATTTTTCTATTTATAAGGCATCAGATAATTCTTCTTTGAGTCAACAGTTCGATGCTTGC |
| | | GCAAGTTGGTGGACACAGGGGCCTGACCCTACTTTTCAGGCTGAATTGGCTCGCGAGATGGGATACACAGCGGCCAGGTTCGGCCAT |
| | | GTAATGTTCCCCGAAAATGTTTATGAACCTGCTCTTAAATGTGCTGAGCTTTTGTTAGACGGTGTAGGAAAGGGATGGGCTTCACGCGT |
| | | ATACTTCTCTGATAATGGCTCAACCGCAATAGAGATCGCTTTGAAGATGGCTTTTAGGAAATTTTGCGTTGACCACAACTTTTGCGAAGC |
| | | CACAGAGGAAGAGAAACACATTGTGGTTAAGGTAATTGCCCTGAGAGGCTCATACCACGGCGATACTTTGGGTGCTATGGAGGCTCAG |
| | | GCGCCATCACCATATACTGGTTTTCTTCAGCAACCCTGGTACACGGGCCGGGGCCTATTTTTGGATCCTCCAACCGTTTTTTTGTCTAA |
| | | CGGTTCTTGGAACATATCTTTGCCGGAAAGTTTCTCAGAAATTGCTCCTGAGTACGGCACCTTCACAAGTCGTGATGAAATCTTCGATAA |
| | | GTCAAGAGACGCTTCAACACTTGCAAGAATCTATTCAGCTTATCTCTCAAAGCATTTGCAGGAGCATTCAGGAGTTAGGCAGAGTGCCC |
| | | ATGTTGGTGCATTAATCATTGAGCCGGTGATTCATGGTGCAGGAGGAATGCATATGGTGGATCCTCTATTTCAGAGAGTCCTCGTTAAT |
| | | GAATGTAGAAATCGTAAGATACCTGTCATATTTGACGAAGTCTTCACTGGTTTTTGGAGATTGGGAGTGGAAACGACAACAGAACTTCTA |
| | | GGATGCAAACCTGATATTGCGTGCTTTGCTAAGCTATTGACGGGGGGTATGGTACCCTTGGCTGTGACATTGGCTACAGATGCAGTTTT |
| | | CGATAGTTTTAGTGGTGATAGCAAATTGAAAGCTCTATTGCACGGACATAGTTACTCTGCTCACGCCATGGGATGCGCTACCGCGGCGA |
| | | AGGCAATCCAATGGTTTAAAGATCCTGAAACCAATCACAATATCACATCTCAAGGCAAGACCCTCAGGGAACTGTGGGACGAAGAACTA |
| | | GTTCAGCAAATCTCTTCACACTCAGCAGTGCAACGCGTTGTTGTCATAGGAACCTTGTTTGCTCTCGAGTTGAAGGCAGACGCAAGCAA |
| | | CTCAGGGTACGCTTCACTCTATGCTAAATCTTTATTGATTATGTTGAGGGAAGATGGCATCTTTACACGACCTCTGGGTAACGTGATCTA |
| | | CCTCATGTGCGGTCCTTGTACGAGTCCCGAAATATGTAGACGACTGCTGACTAAACTGTATAAGCGTTTGGGTGAATTTAATCGTACTTA |
| | | A |
| 157 | Zea mays BioDA nucleic | ATGGTTAGACGCCTGTTTCTTCACCATGCTCGGAGGAGATTGCATTCAGGTACCGCATCAAGTATTCCTCTTTCTACTCCTATATTTGCG |
| | acid (SEQ ID NO: 157) | ATATTCGGGGCTAATACGGGCGTAGGAAAGACGTTAGTCAGTGCTGGGTTGGCTACTGCACTATTATCTTCTCCATCCCCTGCAGTTTC |
| | | AAGTGTTGCTTACTTGAAGCCACTACAAACTGGCTATCCTGTGGACTCAGACGCGAGCTTTGTATTCTCTAGGACACCAGCTTTACTTAG |
| | | AGCATTTCCACCTTGCCGTGCCACCAGGCTCATAGCTAGTTGTCGTACATTCTTCCAATCACCAGCTGTCGAAGCTAAAGCAGAGTCAT |
| | | TGCATGAATCACAGGAAAAAGTGGTGACGTACGGCGCAGGGGCTGCAGAGCAGACTAAGGTCCTGGCATGCTGTACAGCTTACGCCT |
| | | GGCGAGAGCCGGTCTCCCCACACCTAGCAGCCGAGAGAGAGGGCATGGCTGCTGGCGATGATGAAGTAAAGGGATGTGTGGAACAA |
| | | TGGCTATTGGAAGAGGGTATCGGTGAAGGTGGAGAGGTGTGGAAAATACTGGAGACTGCTGGTGGTGTAGCTAGCCCTAGCGCTTCT |
| | | GGTACGTTACAATGTGATCTCTATAGGCCATTTCGTCTTCCTGCAATTCTTGTAGGTGATGGACGATTGGGGGGTATTAGTTCTACTTTG |
| | | TCAGCCTATGAAACACTTCTCTTGAGGGGCTATGATGTCTCAGCCGTCATCCTGGAAGACCGTGAGTTATCTAATGATAAGTTTCTTCTG |
| | | TCATACCTTAGGAATCGTGTTCATGTTTTAATCTTGCCCCAAATACCAGAGGATCCACTTGATGACTTGACCGATTGGTTCAGTGAATCT |
| | | TCAAGCATATTTATATTTTTAAAAGATGAATTGCAAAGTTTCCATTCAAGACGTATTGAAAGACTTAATAGCATGCAAAGGAAGAGTAAAG |
| | | CGTTGCTGTGGTGGCCATTTACCCAGCACAACTTGGTTCCCCAGGATTCTGTTACAGTTATTGATAGCAGATATGGTGAAAATTTCTCCG |
| | | TTTACAAGATTAAGGATAAGATGCTTATACCTCAATTCGATGCATGCGCTAGTTGGTGGACTCAAGGTCCTGACTCCAATCTTCAAATTG |
| | | AGCTTGCTAGAGATATGGGATATGCTGCTGCTAGATATGGTCATGTTATGTTTCCTGAGAATGTTCATGAACCTGCTCTCCATAGTGCTG |
| | | AGGTACTCCTCGGTGGTGTGGGAAAGGGTTGGGCGAGCCGTGTTTATTATAGCGATAATGGGAGCACCGCTATTGAGATCGCTTTAAA |
| | | AATGGCATTTAGGAAATTTTCTTTAGACCACGGTATCATGGCTTCATCTGAAAACTCTACTCGCAACGAAAGAAATATTCAACTCAAAGTT |
| | | TTAGCACTAAATGGATCTTATCACGGTGATACGCTTGGAGCAATGGAAGCGCAAGCTCCGTCCGCTTACACCAGTTTCCTCCAACAGCC |
| | | TTGGTACAGCGGTAGGGGACTTTTTCTCGACCCTCCAACCGTATATATTAAGTCAGAGGTGTATAACATTTCCTTGCCTCAATCTATGCA |
| | | GCATGATCCACAAACATATGGGTTTTTTTCACAGGCCGAAGTTTTTTGCAAGACACGAGATAAGACATTTGCGGCTGATCTGTATTCATC |
| | | TTATATAAAGCAAAAATTGTCAGAGTTTTCTCTTTCCTCATCATCAGAGCATTTAGCCGCCTTGATTATTGAACCTGTTATACAAGGGGCT |
| | | GGCGGTATGCTGATGATTGATCCGTTATTCCAGAGAGTGCTTGTTTCAGAATGTAGATCAAGGAAGATACCTGTTATTTTTGATGAGGTC |
| | | TTTACGGGATTCTGGCGTCTAGGTGTTGAGAGCGCGTCCGAGCTTCTTGGATGCCTACCTGATATCGCTTGTTACGCCAAGCTTATGAC |
| | | TGGGGGGATTATCCCATTAGCCGCTACTCTTGCTACTGAGGAGGTTTTTGAAAGTTTCGAGAGCGACTCAAAATTGACCGCCCTCCTGC |
| | | ATGGGCACTCTTATACTGCACATGCAATGGGATGTACTGCAGCTTTAAAAGCTATGCAGTGGTATAGGGGACCAAGCACGAATTCAAAC |
| | | TTGGATACAGATCATATGAAATTAAAGGAATTATGGGACGGCACTCTGGTCAAACAACTCTCCAGCCTGCCTAACGTGAAAAGAGTTGT |
| | | CTCACTTGGGACTCTTTGTGCAATAGAGCTAAAAGCAGAAGGTGCTGATGCGGGATATGCTTCACTTTACGCTTCAAGTTTAGTACAAC |
| | | AATTAAGGAAGGAAGATGATATCTACATTCGCCCACTTGGGAATGTCATTTACCTTATGTGCGGACCTTGTACCCCACGTGATAGTTGCA |
| | | CACGTCAGCTTCTCAAGGTCCATAGAAGATTGTGCGCTCTCAACTAA |
| 158 | Nannochloropsis | ATGTGCAGCCTCACCATACAAGTATTTGGGGCTAATACTGATGTGGGAAAAACAGTTATAAGTGCAGGGTTGTTGCGTGCTGCTAGTAA |
| | gaditana BioDA nucleic | CAGACCGCTTAGAAGAAGGACTACGTACATTAAGCCTCTCCAGACTGGAAAGATTCATGATGGGTCTTTCGTGCAACGCTACACCTCAG |
| | acid (SEQ ID NO: 158) | CAGATGTTAGTTGCCACACTCTTTACTCCTGGGAAACTCCTTTATCTCCCCACGTGGCCGCTACAAAGGAAGGAAAAACAATATCCTCT |
| | | GAAGAACTTCTCGGAATGTTGCGTACATGTCTTCAGCAGGCCCAAGAGTCACACACAGTCGGTGGAGATGGAGGGGGGGTTAGACAG |
| | | AGAGAGGGTTCAAATGTGGGCGATTTGGTCGTGGTAGAGACAGCTGGAGGTGTTTTGAGTCCAGGACCGTCACCACTCCAAACACAG |
| | | GCTGATCTTTATAGGCCGTTGAGATTGCCAGTGCTACTTGTAGGGGATGGGAGACTTGGTGGAATTTCTTCCACTCTTGCTGCTTATGA |
| | | AAGCTTGTACTGTCGAGGCTATGAGGTTCACGCTATTGCAGTGCTAGAAGTTCCACACTACATTGAGAATGCTGCTGCAGTATCCGAGT |
| | | ACATTACAGGTACTTTGGGTCATGCCGAGACACCTGTTCTGAGCCTACCACATTTGCCAGAAGTTTCATCTGTTAGTTTTTCTACCGGAG |
| | | ACGTTGACAAGGATGTGCTCCTTGGAGGTCTTGATCAGTGGTATCATCAACACGAACACGTCTTTCACCATCTTTTAGACACATTGATTG |
| | | ATAGGGAACGAGTCAGGATTCAAAGACTCCAGAAGATGACTACCCAAGCAGCACAAGTGTTTTGGTATCCTTTCACTCAACATAGTGGA |
| | | CTCTCTGGCACTGATTTACATGTGGTTGATTCAGCTTATGGAGATAACTACACTCTCATAAGCAAATCATCCGAAGATGGTGACGCAAAA |
| | | GAAAGAGTTCCATGCAGCAGGACACTCATTGATGCTTGTGCAAGCTGGTGGACACAGGGCCTCGGACACGGCAGGGTTGACGTTGCT |
| | | CTTGCCGTCGGTGCAGCTGCAGGACGATTCGGACACGTTATATTCCCAGGGAATGTCCACGAACCGGCCCTGAGACTAGCGGAGCTT |
| | | ATGCTGGCTGGACCCGGTCAGGGATGGGCCAGCAGGGTTTTCTTCTCTGATAACGGGTCTACTGCAATGGAAATTGCCGTGAAAATGG |
| | | CACTTAAGAAGAACTCAGTGGACTTGAAGTACGAGAAGGGGTGCTCCAAGGAGGAGTTCAGAGTTGAACAACCAAATGATAAAATTTCA |
| | | GCCACGCCTTTGACCGTAGTGACCCAGAGGGATTGCTACCACGGCGATACTTTGGGTTGTATGGACGCTGCTGCTCCAACCATCTTCA |
| | | ACACGGGTCAGCATCCATGGTACCAACCACGGACTCTGAGCCTGACAGTTCCTACTTTCGGCTATGTATCTGGCCACTTGCAAGTTGA |
| | | GATACCGCCAGAAATCACAAGAGCAGCCGGGCAAAGTGCTCTTCAGTTACCTGATTCCGGCCAGGTACTCAAGTTCCCTGGCGGTTTG |
| | | GCAACATTACTCGATGTTCATGCTAGGGAAGGAAGTGATGCCGCTGTTATTTATAGAAAAGCAATACGAGCTCAGTTCATTGCATTTGAA |
| | | CGTGGTGAGGAAGGAATTCTTTCTTCCAATACTGTTATTCCGCTTCGGGGTGAGGCCGGACAAGGTCATAGATGTCGAAAACTCGGTG |
| | | CAGTGTTGCTAGAGCCCGTATTAATGGGAGCCGCCGGTATGGTGATGGTGGATCCGCTCTTCCAAAGGATATTAACTCTTGAAGGACG |
| | | CGCCTATGGTCTCCCCGTGGTTTTTGACGAAATAGCCTCAGGGCTGTATAGGCTAGGATGCGCCTCCGCTGCTGAACTTCTCGGAGTG |
| | | GCCCCGGATATTGCTTGTTATGGTAAAACCCTGACCGGCGGCTATTTGCCCATGGCTTTGACTTTAACAACTGAAGCTGTATTCGAAGC |
| | | TTTCCATGGTACTAACAAATCTGATGCTTTGCTCCATGGACATAGTTACACCGCTAATCCACTTGGTTGCGCTGCAGCGGTCCATGCAC |
| | | TTAAGGCATATGCAGTGATACATGAGACGAACATTAGAACGTCTCAGGAGTGCTGTCAACGTAAGGAAGATTTTTTCCCTAATAAAAATA |
| | | AATGCAGTAGTCATCACAATATATGCAGATACAATGAGGATCAAGTGGCTGCTCTTTCTAATCTCAGCGGAGTTCGAAGAGCTGTAGCG |
| | | CTTGGAACAGTTCTTGCTGTGGAACTGACAGAGGTGGAATCCTCTAACCGCAAAGGGTACAGTTCTACAGCATCTACACACATCGTGAA |
| | | TGATTTGCGGGAAGCCGGGATTTATGCTAGACCTCTCGGTAATGTTGTCTACTTGATAGCATCTCAGGTTAGTTCCGGTGGAACTTTCG |
| | | AATGCTTGGTGGATACATTGGATCGTGTGTTGAGAAGGCGTGGAGATACTTTGGGTAAGTTTACGCACGACATGACCACTTCTGGTGTA |
| | | GTGATCTGA |
| | BIOA PROTEIN | |
| | SEQUENCES | |
| 159 | Escherichia coli BioA | MTTDDLAFDQRHIWHPYTSMTSPLPVYPVVSAEGCELILSDGRRLVDGMSSWWAAIHGYNHPQLNAAMKSQIDAMSHVMFGGITHAPAIELC |
| | protein (SEQ ID NO: 159) | RKLVAMTPQPLECVFLADSGSVAVEVAMKMALQYWQAKGEARQRFLTFRNGYHGDTFGAMSVCDPDNSMHSLWKGYLPENLFAPAPQSR |
| | | MDGEWDERDMVGFARLMAAHRHEIAAVIIEPIVQGAGGMRMYHPEWLKRIRKICDREGILLIADEIATGFGRTGKLFACEHAEIAPDILCLGKAL |
| | | TGGTMTLSATLTTREVAETISNGEAGCFMHGPTFMGNPLACAAANASLAILESGDWQQQVADIEVQLREQLAPARDAEMVADVRVLGAIGVV |
| | | ETTHPVNMAALQKFFVEQGVWIRPFGKLIYLMPPYIILPQQLQRLTAAVNRAVQDETFFCQ |
| 160 | Cryptosporidium | MPNIFITATDTDVGKTTISALLCLHYGYSYHKVIQSGDLENSDSMTISQLVPNVKIYPEQIKLRKSLSPNLSAEFENKEISLSDFNFDLLSQDNVIIE |
| | andersoni BioA protein | GVGGILVPLSDKLDVIDLMEYFGMITLLVVPSRLGIINQARMALNLLKNRNINTIGFVTNGEYRPDDFNVISRLTNTNHLFHIPTMEIINHESLKSLK |
| | (SEQ ID NO: 160) | IPTDLFCSIEKDKYLGICKKIFEYNTNNQLSYLNLCERDSKIIWHPYTQHGIESEFKCFISGSGSFLIDDEGNKWLDLISSWWVITFGHCDKKLSE |
| | | AITKQVKTLDHIVFAGYTHEPAIKLAEILLDLNSQYFHKVFYSDNGSTAIEVALKAVIHYHQNRGQRNRINWLALEGGYHGDTFGAMSVGYSSN |
| | | YFKPYYEFLFQKIHFIPYPESYDDTTEEQWQIDCELSLNKAKYIISSIGKENIAGIMIEPLIQGASGMRQTRPEFLNKLLSICKDNEIIVIFDEVFTGF |
| | | GRTGTLFAYQQLKYIPDILCLSKGITGGVLPLSVTLFPQYIYDAFVSDNISTAFIHGHSFTANPISCSAAIANLSRINNENTFALIQKINNANSAFILE |
| | | LKKKFEDRIYNYFTKGPLCVFSLKDKYKNYGLNFNIIIKRCLERKYLIIRPLGNRIYLLPPYEIDIETLQQAHKLIIEALFELNI |
| 161 | Agrobacterium | MSRSPVWHPFTQHGLEPPMKRVVSAEGAFLVDEDGNRLFDGISSWWVITHGHRHPAIMAAIRAATEAFDQVIFAEFSHEPAETLARGLIGLAP |
| | tumefaciens BioA protein | NGLSHVFYSDSGSTAVEVALKMALGYFHNRGEKRDRIVVMEHGYHGDTIGTMSTGERGVFNAAYKPLLFGVDRLAFPQAGGEQQTLDMFES |
| | (SEQ ID NO: 161) | FCRSGRIAALLIEPLVLGAGGMKMYGADILAGLKQIAERHGCLVIADEVMTGWGRTGTLFACEQAGISPDILCTSKGLTGGSLPLAATLCSAEIF |
| | | DAHFSTDRRKTFFHSSSYTANPIACAAAVANLQVWRDEPVLQRIGQLQQMLRDNLRRFADDRRFTGIRQMGTIAALDLVVPAGGYLAEAGPR |
| | | MRQLFRERGFVIRPLGNVLYLMPPYCSTGEDLTSAFDAIDEVAGIVTETASLKSRAAGIA |
| 162 | Citrobacter portucalensis | MTTDDLAFDQRHIWHPYTSMTSPLPVYPVARAEGCELILASGEKLIDGMSSWWAAIHGYNHPQLNAAMKTQIDAMSHVMFGGITHAPAIALC |
| | BioA protein (SEQ ID | RQLVAMTPEALECVFLADSGSVAVEVAMKMALQYWQAKGESRQRFLTFRNGYHGDTFGAMSVCDPDNSMHSLWKGYLPENLFAPAPQSR |
| | NO: 162) | MGGEWDELDMVPFARLMAAHRHEIAAVILEPIVQGAGGMRMYHPEWLKRIRKMCDREGILLIADEIATGFGRTGKLFACEHADITPDILCLGKA |
| | | LTGGTMTLSATLTTRQVAETISNGEAGCFMHGPTFMGNPLACAVASASLSLIETGEWRAQVAAIEAQLRAELAPAVDSCWVADVRVLGAIGVI |
| | | ETTHPVNMAALQKFFVEQGVWIRPFGKLIYLMPPHIIQPNQLRRLTQAVNEAVRNATFFSD |
| 163 | Cedecea sp. nfix57 BioA | MTPDDLAFDQRHIWHPYTSMSQPLPVYPVVAGSGCELQLDDGRMLVDGMSSWWAAIHGYNHPRLNQAMKAQIDSMSHVMFGGITHPPAV |
| | protein (SEQ ID NO: 163) | ELCRKLVAMTPAPLECVFLADSGSVAVEVAMKMALQYWHAKGQPRQRFLTFRNGYHGDTFGAMSVCDPDNSMHSLWQGYLPEHLFAPAP |
| | | KCGFGDEWDEMDIVPFARLMAAHREEIAAVILEPVVQGAGGMRFYHPEWLRRLRRMCDREGILLIADEIATGFGRTGKLFACDHAGISPDIMC |
| | | LGKALTGGTMTLSATLATRHVAETISDGEAGCFMHGPTFMGNPLACAVASESLALLAEGDWQQQVPAIEHQLREGLLPLTANENVADVRVLG |
| | | AIGVVETRRPVDMAALQRFFVEQGVWIRPFGRLIYLMPPYIISPEQLEKLILAVTQAVKNPEFFR |
| 164 | Xenorhabdus sp. xeno-1] | MNSSDLEFDLRHIWHPYTSMTHPLPTYPVISAAGVELELSDGRRLIDGMSSWWAAIHGYNHPVLNEAAKSQIDRMSHVMFGGITHPPAIELCQ |
| | BioA protein (SEQ ID | QLVAMTPAALECVFLADSGSVAVEVALKMALQYWQAKGEKRHRFLTLRHGYHGDTFGAMSVCDPDNSMHNLYKGYLPNHLFADAPQSRFE |
| | NO: 164) | GEWQPEDITSFRQLLLQHHNEIAAVILEPIVQGAGGMRIYHPEYLRQVRKLCDEYQILLIADEIATGFGRTGKLFACEHAQIEPDILCLGKALTGG |
| | | YMTLSATLTTRHVAETISQGEAGCFMHGPTFMGNPLACAVASANLKLLSDSQWQPRIQSIETQLKVELLPLKSHTTVKDVRVLGAIGVVEMKQ |
| | | PVDMASLQRRFVQHGVWIRPFGRLIYLMPPYITRSEQLSKLTNAINDVII |
| 165 | Methanobrevibacter | MMNKSEKCVEKDLAHIWHPASQMKDYEDFPPIVIDHGKGVKLVDVDGKEYIDIISSWWCNLLGHCNDEVNDALKEQVNKLDHVLFANFSHKTI |
| | olleyae BioA protein | IELSERLIKVMPKGLNKFSFVDNGSASVECALKMAFQYCGQNGKEYKTRFMCFTDAYHGETLGALSVGALDDYSKVFEPIMIDTIKIQAPDCYR |
| | (SEQ ID NO: 165) | CQHNKNRDSCDCECFEDAEEQFSKYGHETCAMIIEPLIQGSAGMKIYPPLYLKKLRAICDKYDVLLIADEIATGFGRTGKMFAVDHAGISPDIMC |
| | | ISKGLTNGYMAMAVCASTDEIYNGFYGDFTDNVAFMHSHTYAGNPLAASVANATLKIMERDKIIEKANEKAIWLNNRFHEIFDSHPNIGEIRQM |
| | | GLINAIELVEDKDSKKDFDSRERIGYKIYREALKQGLMLRPLGNVMYFNPPLVITKEELEKSLKICKESIDKVLL |
| 166 | Bacillus subtilis BioA | MKQSMIEKSKKHLWLPFTQMKDYDEDPLIIESGNGIMLKDISGKEYYDGFSSVWLNVHGHRKKEMDEAIQKQLEKIAHSTLLGMTNVPATELA |
| | protein (SEQ ID NO: 166) | EMLTGITPEKLTRVFYSDSGAEAMEIALKMAFQYWKNIGRPEKQTFISMQNGYHGDTIGAVSVGSIELFHHVYGPLMFESYKAPIPYVYRSESG |
| | | DPVQCRDECLHALEQLLAKHHEEIAALTIESMVQGASGMIVMPEGFLAGVRKLCTAYDVLMIVDEVATGFGRTGKMFACEHENVQPDLMAAG |
| | | KGITGGYLPIAVTFATEAIYEAFYDDYDKMKTFFHGHSYTGNQLGCAAAIENLRLFESERIVEQVAEKSKTAAEFLHDLKQLSHVGDVRQLGFM |
| | | CGIELVQDKGTKQPYPPEKRMGYKVSLKMRELGMLTRPLGDVIAFLPPLASTTDQLQAMISIMKEAIAEVTGRGF |
| 167 | Pantoea ananatis BioA | MFTSQDAEFDRQHIWHPYTSMQQPLPCYPVVAAKDYHLQLADGRTLVDGMSSWWAAIHGYNHPRLNRALQQQMGQMSHVMFGGITHPAA |
| | protein (SEQ ID NO: 167) | VALCRQLVSMTPDALECVFLADSGSVAVEVAMKMALQYWLGRGETRQQFLTLKRGYHGDTFAAMSVCDPDNSMHSLWRGYLPEHHFAAA |
| | | PQCGFDDEWDARDAEDFIRLIQQHRGQLAAVILEPIVQGAGGMRFYHPRYLQCVREACDRHGILLIADEIATGFGRTGTLFACEHAAISPDIMC |
| | | VGKALTGGTMSLAATLTTRDVADTISLSPAGCFMHGPTFMGNPLACAVASESLAMVQEGDWRQQVAAIERQLRGALLPLRHAPAVADVRVL |
| | | GAIGVVETHQPVNMAALQQFFVSQGVWIRPFGRLIYLMPPYIISPEALDKLVNAIGAALEQPAHFLS |
| 168 | Bacillus licheniformis | MNQSFVEKSKQYLWLPFTQMKDYDENPLIIESGQGIKLKDIDGRVYYDGFSSVWLNVHGHRKKELDEAIKKQLGKIAHSTLLGMTNVPATELA |
| | BioA protein (SEQ ID | EVLIKITPENLTRVFYSDSGAAAMEIALKMAFQYWKNIGKPEKQKFISMKNGYHGDTIGAVSVGAIELFHHVYGPLMFESFKVNVPYVYRSKSG |
| | NO: 168) | NPDECRDECLAELERLLSERHDEIAAMSVESIVQGASGMIVMPEGYLAGVRELCTKYDVLMIVDEVATGFGRTGKMFACEHERVQPDLMAAG |
| | | KAITGGYLPIAVTFATEEIYEAFYDDYNKLKTFFHGHSYTGNQLGCAVALENLRLFESEKIVAQVAEKSKILESLFHDLAALPHVGDIRQLGFMS |
| | | GIELVQSKETRQPYPPEERIGYRVSLKMRELGMLTRPLGDVVAFLPPLASTADDLRAMVSIMKEAIQEVTGRAY |
| 169 | Streptomyces | MPEPLTPGELLALDRQHVWHPYGPMPGKAAPLLVESASGVRLRLAEPVEGQDELVDGMSSWWSAVHGYNHPVLNDAAHGQLERMSHVMF |
| | hygroscopicus BioA | GGLTHEPAVRLAKRLVDISPEPLQHVFLADSGSVSVEVAVKMCLQHWRSLGKPRKQRLLTWRGGYHGDTWQPMSVCDPDGGMHELWQG |
| | protein (SEQ ID NO: 169) | ALPRQIFADAPPAGFDAAPDEAYAAHLRELIGRHAEELAAVIVEPVVQGAGGMSFHSPAYLRVLREACDEHGVLLVFDEIATGFGRTGALFAA |
| | | EHAGVAPDVMCLGKALTGGYLTLAATLCTTAVADGISHGALPVLAHGPTFMGNPLAAAVADASISLLLSQDWGQEVKRIETGLRNGLAPAADL |
| | | PGVREVRVLGAIGVVQLDHEVDMAAATRAAVQEGVWLRPFRDLIYTMPPYITGDDDVARISAAVCAAAREG |
| 170 | Streptomyces | MPDLSVPELLALDRRHVWHPYGPMPGRIDPLVVESASGVRLKLAGGSGELVDGMSSWWSAIHGYNHPVLNEAAHEQLSRMSHVMFGGLTH |
| | viridochromogenes BioA | EPAVRLAKLLVDMSPDGLEHVFLADSGSVSVEVAVKMCLQYWRSLGRPGKQRLLTWRGGYHGDTWQPMSVCDPEGGMHELWTGVLQRQ |
| | protein (SEQ ID NO: 170) | VFADPPPAVYDESYSDHLRSLIERHAHELAAVIVEPVVQGAGGMRFHSPAYLRVLREACDAHDVLLVFDEIATGFGRTGALFAADHAAVTPDV |
| | | MCMGKALTGGYLTLAATVCTSRVADGISRGEVPVLAHGPTFMGNPLAAAVACASIELLLGQDWLAEVKRIEAGLREGLAPVADLPGVRDVRV |
| | | LGAIGVVQLDHAVDMKAATDAAVREGVWLRPFRDLIYTMPPYVTGDADLARIARAVCAAAREG |
| 171 | Stenotrophomonas | MLADPTPSPLAHHWRQRDLQVLWHPCTQMREHPDTLPLVPIARGEGAWLIDHDGNRYLDAVSSWWTNLFGHAEPRIGGAIAAQATQLEQV |
| | maltophilia BioA protein | MLAGFGHEPAITLAERLLALAPRQPGREPLAKVFYADNGSAGVEVALKMAFQYFQNRGESRRTRFIALENGYHGETLGALALGDIPLYRRVYA |
| | (SEQ ID NO: 171) | PLLAEALFAPSPDAYLAEPGQSAADRARQAADGLATLFDQHPGEICAVILEPRLQCAGGMRMHDPVYLQRVRELCDAHGAFMIADEIATGFG |
| | | RTGTLFACEQAGVMPDLMCLSKGLTGGFLPLAAVLATQALYDAFLDDSRERAFLHSHSYTGNPLACAAALTTLDIFRDDDVIARNRSIASVMG |
| | | TLAAPFNDHPHVADVRQAGMVVAFELSRDGNKRTPFDPAMRLGLHAYKAALKRGVVLRPLGDVLYWMPPYCVDDEQLELLAHTTLAAIDEAI |
| | | ACA |
| 172 | Pseudomonas | MGLNNQWMQRDLAVLWHPCTQMKDHEQLPLIPIKRGEGVWLEDFEGKRYLDAVSSWWVNVFGHANPRINQRIKDQVDQLEHVILAGFSHQ |
| | fluorescens BioA protein | PVIELSERLVKMTPEGLNRVFYADNGSSCIEVALKMSFHYWLNRGMPAKKRFVTLTNSYHGETMAAMAVGDVPLFTETYKALLMDTIKVPSP |
| | (SEQ ID NO: 172) | DCYGRPEGMSWEEHSRNMFAAMEQTLAENHDSVAAVIVEPLIQGAGGMRMYHPVYLKLLREACDRYGVHLILDEIAVGFGRTGTMFACEQA |
| | | GIRPDFLCLSKALTGGYLPLAACLTTDDVYSAFYDDYPTLRAFLHSHSYTGNPVACAAALATLDIFEQDNVIESNKALAQRMASATAHLVDHPN |
| | | VSEVRQTGMVLAIEMVKDKATKEAYPWQERRGLKVFQHALERGALLRPLGSVVYFLPPYVITPEQIDFLAEVASEGIDIATRDSVSVAVPKDFH |
| | | PGFRDPG |
| 173 | Bacillus thuringiensis | MIREITDLQARDLRHIWHPCSQMKDYEAFPPIVIKKGQGVWLYDEHDKRYLDAVSSWWVNLFGHANPRISQALSHQAFTLEHTIFANFTHEPAI |
| | BioA protein (SEQ ID | QLAEKLVALSPPGLHKVFFADNGSSAVEVALKMSFQYHMQTGKQTKTRFLAFTDAYHGETVGALSVGGVGLYNEVFEPLLLDTVRAQGPDC |
| | NO: 173) | FRCPFQKLPESCDAPCSHFVEEQLKMHHEEITAVIIEPLIQAAAGMKMYPPIYLQRLRALCSQYDVHLIADEIAVGFGRTGTMFACEQASITPDF |
| | | MCLSKGITGGYLPLSAVLTSDQVYNAFYDEYGTMKAFLHSHSYSGNTLACRVALEVLTIFEEEQIMDMIQRKGQQMRQLAMEAFGHMPYVGE |
| | | YRQVGLVGAIELVADRQTKTPFASEERIGYQIYQQALEKGLLIRPLGNILYFMPPYIITNEEMTFMINTTKETIEQFFKDRGE |
| 174 | Candidatus midichloria | MINLAKIDKQIIWHPITQDKIADLPVIIKEGMGPYLIGQDNKKYLDLISSWWVNLYGHANPHIAKAIYEQALKLEQVIFAGFSHEPAINLCMGLQKIL |
| | mitochondrii BioA protein | PQPLKRFFFSDNGSSATEIAIKMAFQYWFNQKDYNKKSYISFAGGYHGDTIGAMSVGQSPLHRTFGALLFKNHHIPFPQTWDGDLDVELKEEI |
| | (SEQ ID NO: 174) | SLNALQEIIKKHKDEIIALMVEPLVQGANGMQICRPSFLEEICKLTREAGILVIFDEVMTGFGRTGKNFAFEHIDFVPDIICLSKGLTGGFLPLALTI |
| | | ATEKIFEAFLGNNMQVAFTHSHSYTANPLGCAAAIASLELLVKAETTENISTIKEAHTIGLQKLLATEAKLEKFRQIGTIAAFDMVLPDDLDLFKVA |
| | | LSLRRKFIEAGYIIRPLGKTLYLLPPYCTSAQELEQFYENISAIIAEEF |
| 175 | Thiofilum flexile BioA | MDWVQIDRQHVWHPFTQEQTAPLPVEISSAQGAWLYAKEGQRFLDLISSWWVNLHGHAHPTIAAAIAQQAQTLEQVMFAGFTHQPAAELAY |
| | protein (SEQ ID NO: 175) | ELVQRLPTGLTRVFYSDNGSTAIEVALKIALQYWRNQGQQRKRFAVFDGSYHGDTVGAMSVGQGSQFFTHYQALLFQVDVLPYPLTWERDP |
| | | HVEQKEAVALQAFDAYLQRHAHEVAGILIEPLVQGASGMRMCRPQFLQGLSQRAKAAGVLLLFDEVMTGFGRTGQWFASQTAQVTPDIICLS |
| | | KGITAGFLPLSVTVCSETVYQAFLGETFDRAFVHGHSFTANPLGCAAALASLQVFKTENTLAKLPQIEHWHRQQLEWLLQHPKVEQVRICGTI |
| | | AAFNVKATETGYASSVSEHLRTFFMERGILLRPLGNTVYLLPPYCITEDELISGYQVIERALDALI |
| 176 | Panacibacter | MELAAKDRLYVWHPFTPQKDMIAPVPIVKGKDTLLIDESGNTYIDAISSWWVTLHGHGNEYIAKKIYTQALKLEQVIFAGFTHEPAVELAEKLIRI |
| | microcysteis BioA | LPAGFAKVFYSDNGSTSTEVALKMALQYWWNTKTEGKAVDTENSPPDGVTRSHNKRNKILAFRNSYHGDTFGAMSVSDRSVFTLAFHDLLF |
| | protein (SEQ ID NO: 176) | EVTFIDTPSKENLRQSLDLINRQGNEIAAFIYEPLVQGAGGMKMYDAGLMNELLATVKRYDIICIADEVMTGFGRTGKLFASEFLAHKPDIICLSK |
| | | GLTGGTMALGVTASTDSIYQAYVNDDKLKTFFHGHSFTANPLACTAAIASLELFEQYSVQDKIAWLAKENEQFGKRLQSNYGSICRNIRVLGTI |
| | | LAFDLAIGRNEYLNNIAGVVTDMAMKAGVYIRPLGNVVYIMPPYCITAYELEKVYNTIEAIISSISTNA |
| 177 | Spirosoma linguale BioA | MNDLAERDRAVIWHPFTQMQTAPLPIPIVRGSGSVLYGADGREYLDMVSSWWVNIHGHAHPHIAQRVSEQLHTLEHVIFAGFTHQPAVELAE |
| | protein (SEQ ID NO: 177) | RLLAILPANQSKVFYSDNGSTAVEVALKMAFQYWHNLGKPRKKIVAFENAYHGDTFGAMAVSGRSAFTAPFTPFLFDVVYLPVPIPGQEEIVL |
| | | QQAEALFSEEVAAFIAEPLVQGAGGMVMYQPDVLDTLFRLARDKGALIIADEVMTGFGRTGKLFASDYLAEKPDMMCLSKGLTGGTMALGVT |
| | | SCAESIYNAFLSDDKHKTLFHGHSFTANPLACTAALASMDVLLLSETQANIERITASHAAFANKLAAYPTVENIRQHGTLLAFDIKAGEQTSYFN |
| | | NIRDTAYNFLLDRGILMRPLGNVLYMMPPYCTTDEQLAYTHEQIQAFLATL |
| 178 | Prochlorococcus | MKSQISTNINQDWHPHIWPPFTQITNSKPQIEVTHGKNALIYTKNPQQELIDGISSWWVTLHGHSNDYIANAIFHQAKTLEQIIFADFLHPQAKLL |
| | marinus BioA protein | SERLSSLTKLERLFFSDNGSTAVEVALKIAYQSWQNQGEIRNQIIAFDGAYHGDTFGAMALGERNIFNENFDNLMFPVKRVPWPSTWIDDEKV |
| | (SEQ ID NO: 178) | KIKEKQAIQILNNLLKKPTVAVIIEPLVQGAGGMNMVRPEFIKKVSEVVKNNNSLLIADEVLTGFGRCGSLFAFQKANIIPDLISISKGLTGGFLPM |
| | | GITLAKETIFQAFISDSPKKTFWHGHSFTANPLGCAAANASLDLLENDPVKYISFEAKHSYHLKKIKKLPFVKNIRFTGTIAAFDIEIGNNEGYLNNI |
| | | GKKIKALAIKKGLFIRPLGNVIYLLPPLCITDKQLEKSYRIIFEILSDL |
| 179 | Chitinophaga | MQQKRCWTYLHSIDRYKPFCTNFAPAQKQATMQTKSLSARDLDVIWHPYTQMQTAPAPIGMVRGQGALLFDENDQSYIDATSSWWVNIHG |
| | polysaccharea BioA | HAHPHIAQHLAAQALQLEHCIFAGYTHPPAVNLAERLLQVLPGNQRRVFYSDNGSTAIEVALKMAIQYWDNQGIRKRKILAFNNAYHGDTFGA |
| | protein (SEQ ID NO: 179) | MSVSGRSVFTRVFDDLLFDVYFIDVPNAANLSQLIATIDALQPEDIAAFVFEPLLQGSGGMIMYEADAFDQLLQYCKSRNILLIADEVMTGFGRT |
| | | GKNFAMEYLQTMPDMICLSKGLTGGTMALGVTTSTAAIYEAFLSNDKLKTLFHGHSFTANPLACTAALASMDLFMDPACAANRQRIHEKHQAF |
| | | LQELQPIAVVKHPRLLGTILAFEISTTNADGYTNSLADELHRFFRARRIMLRPLGNTLYILPPYCITNEQLEQVYQSIRELLKKLKA |
| 180 | Chitinophaga filiformis | MLWYPYTQMRQINELPKMVSGEGVMMHLEDGRSLIDGISSWWAVIHGYNHPALNAALLTQANKFAHVMLGGMTHNPALDLAAKLVSITPQG |
| | BioA protein (SEQ ID | LNHVFFSDSGSIGVEVALKMSIQYWKNIGYNGKSKIISLRNGYHGDTFKAMEVSDDSDFTRAFSDVLRRGYILDIPEGGFDADAALVKQATDKL |
| | NO: 180) | EALLRREHHNIAAFIVEPIVQCAGGFNIYSPLYLKAARELCTKYNVLFVFDEVATGFGRTGKLFAAEHAGVTPDIMILGKALTAGYMGHAATLAT |
| | | SAVFDSFLGDNYEKALMHGPTFMANPLACAVALRSISIIEDEQYLQKIAKIQSIIREQFDTFSSPAIVAKRSIGAIGALEMKDAACLSGFKEFSQQ |
| | | RGVWLRPIGNVLYLMPPYIISEKELLTILQVIKDWIQQIK |
| 181 | Pedobacter hartonius | MTLTQRDAESIWHPYTQQLNARPPLPVLSGKGAYLYDEEGCRYIDAVSSWWVTLHGHSHPYIVERVTAQLQQLDQVIFAGFTHEPAIALAENL |
| | BioA protein (SEQ ID | LAILPEGQKKVFYTDNGSTAVEVALKMCVQYWFNKGKSRKKVLAFNNGYHGDTFGAMSVSGRSAWTAPFDNLLFEVIFIDTPTEENLPVLKEII |
| | NO: 181) | SVHAAELACFVYEPLVQGSAGMLMYEAPALDQLMAHCLEEGVLMIQDEVFTGFGRTGKNFAADHLKTQPDVMCFSKGLTGGTMPLGVTTCT |
| | | EDIYNAFLSEDKLKTLFHGHSFTANPLACTAALASIELLLQHEAQDNIRRICNRHAEFSLKICGHQKVAAVRQTGTIIAIEWQTESGTSYFSKLRN |
| | | LLYDYFMDKGILMRPLGNVIYILPPYCISDEDLKKIYQEIQNALEIF |
| 182 | Fluviicola sp xm-24bin1 | MSDLISKDQQHVWHPFTQHHTAGDPLPIVKAEGVYLYDESGKSYIDANASWWVNTHGHGHPHIGKALSEQFQTVDHIVFAGATHPKAVELAE |
| | BioA protein (SEQ ID | RICGHLPDHFQKVFFSDNGSTAVEIALKMAIQYWHNKGENKQRFLAMHGSYHGDTFGAMSVGQRGYFNAPFEPLFFDTDYLDFPNGSNESE |
| | NO: 182) | ILERAESLLSSGDFAGLIVEPLVQGAAGMRMYAPEFLDQLTAIAQKHGVLVIFDEVMTGFGKTGKLFSMDHCQNKPDFVALSKGLTAGVMALG |
| | | LTVTSDKIYEAFLGEETTKALLHGHSFTANPIACSVACANLDLFEREETWQNIDAMVQWNQAFAEELTQFDFVVNIRQQGTILAFEIQESGEAS |
| | | YFSDLKTRAYEFFLERGVLLRPLGNVIFVNPPFCITEEEFGKVKEVILSFLKSL |
| 183 | Flavobacterium foetidum | MGLVEKDSQYLWHPYTQHKTASAPIAITKAEGALFWDENNKEYIDAIASWWVNPFGHSNKTIADAIYKQLTTLEHVLFGGFTHEPAVKVAEKL |
| | BioA protein (SEQ ID | MEILPKNQKKIFFSDNGSTAVEVAIKVALQFFFNKGEKKTTIIAFENAFHGDTFAAMAASGISFYTMAFQGMFIDVVRVPVPVKGKEQASFDALE |
| | NO: 183) | EVIKKHNCAGFIFEPLVQGAAGMVMYEPEALDKLIKICKKNNILTIADEVMTGFGKTGKNFAMDHATEQPDMICLSKALTGGTIPMAITTFTQDIF |
| | | EAFYDDDINKALFHGHTFTANPTGCAAALASIDLLQTPEMQENITRIHKSHLLFQEKIEKHPKVITARTLGVIFAVEIKSDSEETYYGSMRTKLYN |
| | | FFIENGVVLRPVGNIVYILPPYIMTDKQLEKVYKTIEEAIEMV |
| 184 | Chroococcidiopsis sp. | MSVEEIHPSYPIWHPSTQMKTSPPPLKVVKGQGVMLELEDGRQILDCISSWWVTIHGHSHPILANALYQQAQQLEHVIFAGFTHEPAEQLARK |
| | CCMEE 29 BioA protein | LLNHLPKSLTRVFFSDNGSTAVEVALKMAYQYWYNQGETQRTSFIGFEGGYHGDTLGAMSVGGSTSWWQTFQLLMFSIDVVPFPATFDRDP |
| | (SEQ ID NO: 184) | NVEAHEAQSLETITRLLKQNPDRYAGIFIEPLVQGAAGMRLCRPQFLQELESLARSFDVLLIYDEVMTGFGRTGEFFACLKSATTPDILCLSKGL |
| | | SGGCLPLAVTLATEDIYRAFYSDDIHKAFLHGHSYTGNPLACATGVASLELLEQNPEFRAIEHQHRYYLEKWLIGHPKVEQLRTCGTIAAMEVR |
| | | TEDQSSYFNAIGPTLRSRFLEEGFLLRPLGNTIYIMPPYCITPNQLESIYQAIRRVLDTL |
| 185 | Tenacibaculum | MNLTERDKKHLWHPLTQHQLYPNHLAITKAKGAILYDDKNNEYIDGIASWYTCMYGHCNEYITSSVYKQMQQLDHVVFAGFTHEPAIKLSEELI |
| | adriaticum BioA protein | KILPENQEKVFFSDNGSTSVDVAIKMALQYHFNQGEKKGKIIALEDGFHGDTFGAMSVSGLSVYNGPFEDFFIDVARIPVPTQENFEHVKEEFI |
| | (SEQ ID NO: 185) | KLITEHNVAAFVYEPLVQGAAAMKMFDGNLLNELIKIAQENNVLAIADEVMTGFGKTGKNFASEYLEHKPDIICLSKSLTAGLVPMAITSCSQKV |
| | | YNAFLSDDIGKGFFHGHTYSANPVACSAALAGIELLQSEEIQGNIQRIIKSHQEFDSKIKKHPKVKSTRQKGIIYALDLNVEMERYGDLRYKLFNF |
| | | FMENGVCLRPLGNTIYILAPFVITNEQLEKIYKTIEKALEIL |
| 186 | Pseudobacteriovorax | MSYKLFVTGTDTDVGKTFVSQSLLIGALRQEFDLSYWKPVQTGHPDSDLASIKKQIPDLKARPTSFVYKEPASPDQAASLELRPAPRLEQLLEE |
| | antillogorgiicola BioA | LQQIQENTLIEGAGGLLVPLNEDNETWLSFLKKAGIPCIVVARTGLGTLNHTALTLRTLDLHNVPVAAVILSGKKHEANERSLSRMFPEHRFLHL |
| | protein (SEQ ID NO: 186) | DSIEDTHSSSYTEACQRLWCDLTASAVSSPSSLLALDQKHCWHPYTQHKGAATPLEIKRAEGPWLHVSTGEKLIDGTSSWWSNTIGHGRPEI |
| | | AEAIFKQQQTIDHIIFAGATHEGAVKLSQRLSQLTEHQFPRVFFTDNGSCAVEVGLKIAAQMAFNRGDKGRTKFLSLEGAYHGDTFGAMSVGG |
| | | TEGFHGPFSPFQFESLKIKPVTAHPSAICPEGAAARETEIQKLKALFSSCGHELAAVVLEPMVQGASGMNMHDLTWLRSLVAIAKDHQVPVIF |
| | | DEVFTGLGRCGAWFAYQKAGVEPDIICLAKGLTGGNLPLAVTMTTDRIFEQFYSDDKRKALYHGHTYTANAICCAAANATLDIYEREDLIKRSA |
| | | EIESRFKSWIKQHEAELGLAQARAMGSILAWEIPGSGLGDYFNPIAARVPEEARKFGLFLRPLGNTMYFLPALTITDEELSFCLNALEKTVRAIS |
| | | SYNA |
| 187 | Texcoconibacillus | MTDANKVMELSKEYLWLPFTQMKDYEKDPLIIEEGEGIKLKDIYGKEYYDGYSSLWVNVHGHRKQEIDDAVKEQLDRIAHSTLLGATNVPATLL |
| | texcoconensis BioA | AERLIQISPPNLQRVFYSDSGAEAIEIALKMAFQYWYNNGNEKKTKFVTFSDGYHGDTVGAISVGAVDLFHKIYNPLMFNSFKVPFPDTYRHPS |
| | protein (SEQ ID NO: 187) | DDPQEVMRESLSALKETLDKHHEEIAGLIMESKIQGAGGMKIMTDGFFKEVENLCRTYGILLIVDEVATGFGRTGKMFAVEHEEIRPDIMTVAK |
| | | GITGGYLPIAATITSEEVHQAFYDDYTKLKTLFHGHSYTGNQLGCAAALANLDLFEKENIVASVEQKASVVQEALKPVAEQAGVGDVRQLGLM |
| | | CGIELSKDKITKEPFPFEERMAYHTTLTMRKNGMLTRPLGDTVIFMPPLAAEESDLKAMVGIIEKSIEETITD |
| 188 | Nitrobacter sp. 62-13 | MPASPVWHPFTQHAVQPDAIPIARGEGAWLETVDGRRIFDAISSWWVVTHGHRHPRIVQAIKQQAERLDQVIFAGFTHEPAERLARLLVAMT |
| | BioA protein (SEQ ID | PPGLDHVFFSDSGSTAVEVALKMALGFWRNSGERRSRILALEGAYHGDTIGGMSVGERGVFNATYDPLLFDVERIPFPAAGREQVTLDAITTA |
| | NO: 188) | CRNAGVAALIVEPLILGAGGMLIYPPWVLTEMKRICEAHGVLLIADEVMTGWGRTGTRFACEQAGVTPDIACYSKGLTGGSLPLAVTLCRAAIF |
| | | DAHYSTDRSKTFFHSSSYTANPIACAAACANLEIWQDEPVMERIAAIARAHTRGLDRFRGDARFANVRQIGSIAALDIVTDDAGYLAGIGPRLY |
| | | DNFLRRGLLVRPLGNTIYLMPPYCSTPQELERVFDAIGEIVDGIG |
| 189 | Wigglesworthia | MNINEVNFDRYYIWHPYSSITKPLKTYHVVSAKKCKLKLINGKKLIDGMSSWWTVIHGYNHPKINQAIINQIKKVSHVMFGGITHSPAIKLCKKIIK |
| | glossinidia BioA protein | LTPKGLECVFLSDSGSVSIEVALKMALHYWFSKNERKKVKFLSLRGGYHGDTFGALSVCDPKNSMHNIYQGYLPNQEFTNTFDRKFNEKWES |
| | (SEQ ID NO: 189) | GDDLNLVKLIEKHHKKLAAVIIEPIVQGVGGMNFYHKQYLISIKKLCKFYKILLIVDEIATGFGRTGKLFACNYSKITPDILCIGKALTGGAITLAATIT |
| | | KRKVSETICNKKTPEFMHGPTFMANPLSCAASCANLSLIEEGKWIQQVKFIYQEMCKYLYPLIEHSYVKDVRILGAIGVVETKFFVNLENIQNWF |
| | | VRHGAWIRPFRKLIYLIPPYIISSYELKFLIKLISLSLNDDKNFLYIKN |
| 190 | Methylomarinum vadi | MQTLTTEQLLNLDRQHIWHPYTSIPARHPLYPVESASGVKLTLADGRELIDGMASWWSAIHGYNEPRLNAAAHRQIDILPHVMFGGLTHSPAV |
| | BioA protein (SEQ ID | ELVERLLAITPEALNRVFLCDSGSVSVEVALKMAIQYWQAQGKTAKSRFLALRGGYHGDTFGAMSVCDPVNGMHHLFSEVLPQNFFAPRPQ |
| | NO: 190) | SRFGGDWVADDINAAETIIAEHAGQIAAVILEPIVQGAGGMWFYHPNYLSEMRKLCDRYQVLLIADEIATGFGRSGRLFACEHAGIVPDILCLGK |
| | | ALTGGYLTLAATLTTENIAETISAGEAGCFMHGPTFMGNPLACSVAAASIDLLLDSDWQANIVRIEQRLTTGLAPCRNLPGVADVRVLGAIGVV |
| | | EMREAVDVPQLQEKLIEHGVWLRPFGRLVYTMPPYIITDEQLNALTTAVYRSLAG |
| 191 | Flocculibacter | MKTNQQFDLEHIWHPYTSMLKPLPTYQVKQAKGVFIELETGEQLIDGMSSWWSIIHGYNHPVLNQAIHEQTAKMAHVMFGGLTHQPAIDLCR |
| | collagenilyticus BioA | KLVEIAPDGLNKVFLADSGSVAVEVALKMAIQYWRGKNKPQKNKMVALKHGYHGDTFGAMSVCDPENSMHTIYSDILPNHYFVEAPKTQFDE |
| | protein (SEQ ID NO: 191) | NLPSDNLVNDVAAMTQLLQHHHDDIAAVILEPIVQGAGGMRMYHPAYLASVKQLCEQYDVLLIADEIATGFGRTGQLFGCHHAHITPDIMCVG |
| | | KALTGGYLSLAATLCTDEVAMGVSTGDAGVLMHGPTFMGNPLACSVANASLNLLESSNWQSSVSHIEQQLKQSLIPLASLKKVHDARVLGAI |
| | | GVIEMEESVDVADVQKRFIRHGVWVRPFGKLIYIMPPFVISNSELTVLINAMVTVVTELSES |
| 192 | Leptolyngbya ectocarpi | MHPLSTDQLLAIDRQHVWHPYAAMPNSLPIFPVKSAHGVHIELEDGRTLVDGMSSWWTCIHGYNHPRLNRAAREQMERMSHVMFGGLTHQ |
| | BioA protein (SEQ ID | PAVQLAQKLVDLTPEPLQQVFFCDSGSVSVEVAMKMAIQYWYNLGQPEKHRFLTVRGGYHGDTFDAMSVCDPVNGMHHLFNQVLRQQYFA |
| | NO: 192) | DVPKIPFWGVWEDTDIASFEQHLVENSGQIAAAIFEPVVQGAGGMRFYSPDYVRRARELCDQYGVLLILDEIATGFGRTGQLFACDHANIAPDI |
| | | LCVGKALSGGFMSLAATLTTPHISQVFAQGGSGVFMHGPTFMGNPLACAVALENISILESYDWRAQIQRIESQLKAELAPCRESSLVKDVRVL |
| | | GAIGVVELHEPVDMAVIQPQCVDHGVWLRPFGRLVYTMPPYIIEPADLSRITQAMVALTR |
| 193 | Psychrosphaera | MNKKTTTHTTIDINFDKEHIWHPYTSMTNPLPCYPVKSAEGVYIELEDGTKLIDGMSSWWSVIHGYNNEHINEAISSQLKKMSHVMFGGLTHEP |
| | aestuarii BioA protein | AVNLAKHLVELTPDGLNKVFLADSGSVSIEVALKMAIQYQLGKGHIGRNRFIALKHGYHGDTFGAMAVCDPLNSMHSLYSGMMPQNYFIDSPT |
| | (SEQ ID NO: 193) | SKPTDSFQHNELAELADVLECRSDTVAALIIEPIVQGAGGMRFYHPDYLSGAKKLCEKYGVLLIADEIATGFGRTGKMFACEHANITPDILCVGK |
| | | AITGGYMTLAAAICTDEVAITLSNSRAGVLMHGPTFMGNPLACAAANASMELLKAGTWENQVKNINRILNQELNSANQLERVTEARVFGAIGVI |
| | | EMKEAVDVASIQKQFVDKGIWVRPFGKLIYVMPPYIISEIELTELLQKMLLVVKSL |
| 194 | Fragilariopsis cylindrus | MSSPIDLIQKLDQEHVWHPYAAMPNSDPCFPIKSASGCDIHLMDGRTLTDGMSSWWACIHGYNHPIMNQAAKDQIDKMSHMMFGGLTHEPA |
| | BioA protein (SEQ ID | VSLAKTLADTLPANQTCQNENERLDKVFFSDSGSVSVEVAMKMALQYHYNLIHTGASSSTTTTTTSSRPRKSKFLTIRGGYHGDTFEAMSVC |
| | NO: 194) | DPVNGMHHLFQSIIPEQIFCSRPEIKFDEPWDEIDIQEVSRTMEDRHDEIAAVILEPIVQGAGGMRFYSPQYLHRLRELCDEYNILLIFDEIATGF |
| | | GRTGKLFAMDHAPGVLPDILCVGKALSGGFLSFAATITSLKIANVFSQGPAGVLMHGPTFMANPLACAVSNASLDILKSYDLPKMIGSIEKQLIE |
| | | ELEPCKESPFVQDVRVLGGIGVVEMKEPIDMKVMQSKIVEEGVWLRPFGKLLYTMPPFIIQKHELRKITKVMVSIATRRIR |
| 195 | Deferrisoma camini BioA | MPELSFDWIAFDREHLWHPYTAVESPVPVFPVVGARGVRLELADGRTLIDGMASWWSAIHGYNVPELNRAAAEQLERMSHVMFGGLTHPPA |
| | protein (SEQ ID NO: 195) | AELGRLLVEITPEPLDRVFFCDSGSVAVEVAIKMALQYWQARGRPEKRRLLTIRQGYHGDTFGAMAVCDPDTGMHRIWRGILPQHLFAPAPR |
| | | CRFGQPWDEGDIAEFRRLIREHRHEVAAVILEPVVQGAGGMWFYSPQYLRRVRELCDEHEVLLIADEIATGFGRTGRLFACEHADVAPDILCV |
| | | GKALTGGYLTLAATLTTTRVAQGICSGEPGVFLHGPTFMANPLACAVAAASVRLLLEGPWQERVARIGQGLRQGLAPCAALPGVRDVRVLGA |
| | | IGVVELEEPVDMGRVQPRFVEEGVWVRPFGRLVYVMPPYVISPQDLAFLTAAICRVVAEIGG |
| 196 | Chlorobaculum tepidum | MTIDLDFDRCHLWHPYTSMADPLPVWPVKRASGVMIELEDGRKLIDGMSSWWAAIHGYNHPVLNRAVTEQLGRMSHVMFGGLTHEPAIELG |
| | BioA protein (SEQ ID | KILTSLLPDPLDRIFFCDSGSVAVEVAIKMALQYWLAAGKPGKKRLLTVRSGYHGDTFMAMSVCDPVTGMHSLFSGAVPEQLFVEAPACGFN |
| | NO: 196) | EPWREEAIDKMRQALEDHANTIAAVIIEPIVQGAGGMRFYSPHYLRRLRELCTEHGVLLIFDEIATGFGRTGKLFAMEYASVTPDIVCLGKALTG |
| | | GYMTLAATVTTGHVADTISGGNPGLFMHGPTFMANPLACAVAVASLKLLLSGDWQSTVWRIERQLAEELAPCTGMTGVRDVRVLGAIGVVEL |
| | | DRPVDMAKIQQAFVERGIWVRPFGRLVYLMPPFIIRDNELTRLTSVICEVIGAEYR |
| 197 | Chlamydia pneumoniae | MDKQSSGNSGCIWHPFTQSALDSTPIKIVRGEGAYLYAESGTRYLDAISSWWCNLHGHGHPYITKKLCEQAQKLEHVIFANFTHEPALELVSK |
| | BioA protein (SEQ ID | LAPLLPEGLERFFFSDNGSTSIEIAMKIAVQYYYNQNKAKSHFVGLSNAYHGDTFGAMSIAGTSPTTVPFHDLFLPSSTIAAPYYGKEELAIAQA |
| | NO: 197) | KNVFSESNVAAFIYEPLLQGAGGMLMYNPXGLKEILKLAKHYGVLCIADEILTGFGRTGPLFASEFTDIPPDIICLSKGLTGGYLPLALTVTTKEIH |
| | | DAFVSQDRMKALLHGHTFTGNPLGCSAALASLDLTLSPECLQQRQMIERCHQEFQEAHGSLWQRCEVLGTVLALDYPAEATGYFSQYRDHL |
| | | NRFFLERGVLLRPLGNTLYVLPPYCIQEEDLRIIYSHLQDALCLQPQ |
| 198 | Pedobacter | MSDLVSRDLEAIWHPYTQMKTAAKPIPIVKGEGVYLYTEDGKKLIDAVSSWWVNIHGHSHPYIAEKVFEQLQVLEHVIFAGFTHPQAVKLAENL |
| | psychrophilus BioA | LSIIPKNQAKIFYSDNGSTAVEVAIKMCLQFWSNQNQHRTKILAFKNSYHGDTFGAMAVSARSAFTAAFDSLLFDVEFLDLPNENNLEVLKSQIS |
| | protein (SEQ ID NO: 198) | NLKSELACFIFEPLVQGAGGMLMYEAEDLDELILHCKKENVLTIADEVMTGFGRTGKLFASDHLTQKADLMCFSKGLTGGTMPLGVTSCTQEI |
| | | YDAFLSDDKLKTLFHGHSFTANPVACAASNASFDILMQAETHESLKRISKKHHQFKEEIELNPKVKNVRQCGTILAMEWNIEATSYFNNIRDTLY |
| | | NFFLDKGIILRPLGNILYIMPPYCISNDDLDYIYNQIREALIKF |
| 199 | Pseudopedobacter | MQSELIERDRKVNWHPYTQMKTARHIPIIRGEGAYIFDANGKRYIDAVSSWWVTLHGHAHPYIAKKVAEQLNTLEQVIFAGFTHPVAIELSERLL |
| | saltans BioA protein | NLLPDNQEKVFYTDNGSTAVEVALKMCIQYGHNQGVRKNKILAFKNGYHGDTFGAMSVSGRGLWTAPFGELLFDVLFIDAPTKENLSGTKSFI |
| | (SEQ ID NO: 199) | EKHASEIACFIYEPLVQGAGGMLMHEADDLSELMKFCRSKGILLIQDEIFVGFGRTGKLFAANHLSAQADIMCFSKGLTGGTMPLGITTSSQKIY |
| | | DAFYSNDKTKALFHGHSFTASPLACAAALASMDLLLQEETQQNIQRICKQHQQFVNTLVKHPKIKNVRQQGTIFALEWKIEEDTSYFSDMHDIL |
| | | YPFFLQRGILMRPLGNIIYLVPPYCISQQDLEYIYFCILDALETL |
| | MTP RELATED | |
| | SEQUENCES | |
| 200 | Mitochondrial signal | MSVSCCCRNLGKTIKKAIPSHHLHLRSLGGSLYRRIQSSS (SEQ ID NO: 200) |
| | peptides (MTP) | |
| 277 | | MIPVTATLIRHRLRHLRHRIRF (SEQ ID NO: 277) |
| 278 | | MVRRLFLHHARRRLHSGTASSIPLSTPIFAIFGANTGVGKTLVSAGLATALLSSPSPAVSSVA |
| | | YLKPLQTGYPVDSDASFVFSRTPALLRAFPPCRATRLIASCRTFF (SEQ ID NO: 278) |
| 279 | | MASLALTRRRFNPVSRLSLHKCRSTF (SEQ ID NO: 279) |
| 280 | | MHLLLLLPLRRRCTNPIAPRIAHQSRFLVSTAGACSPLPRHLLSGIWGRCL (SEQ ID NO: 280) |
| 281 | | MLPRLLLRSRHRRRY (SEQ ID NO: 281) |
| 282 | | MPSCPLLPLLPRRRPPPSPLLLLLLSRSLHSS (SEQ ID NO: 282) |
| 283 | | MSAPIARRASSVARGRTRWLTSTSIERSREWFVRS (SEQ ID NO: 283) |
| 284 | | MFHPPLLFTLSRHFLRRHHHRH (SEQ ID NO: 284) |
| 285 | | MYFLISLHRRGHRHLRHVKDL (SEQ ID NO: 285) |
| 286 | | MLTLSMRMRPRTLMKMSFSTRAGLGQLGCLT (SEQ ID NO: 286) |
| 287 | | MAPVPALLWRSLRTHQVYGANTDVGKTIFSTVL (SEQ ID NO: 287) |
| 288 | | MLRRLLLRHARRLHY (SEQ ID NO: 288) |
| 289 | | MSPVTATLLRHRLRHLRHHHIRLN (SEQ ID NO: 289) |
| 290 | | MLLRRHHHHRLFRLRLLLLHHHHHQFQSF (SEQ ID NO: 290) |
| 291 | | MLRLLRHARRH (SEQ ID NO: 291) |
| 292 | | MFRFPSVLLSRRLHRREL (SEQ ID NO: 292) |
| 293 | | MLRRLLLRQARRRHCSSSFSSALP (SEQ ID NO: 293) |
| 301 | Escherichia coli BioA | MSVSCCCRNLGKTIKKAIPSHHLHLRSLGGSLYRRIQSSSMTTDDLAFDQRHIWHPYTSMTSPLPVYPVVSAEGCELILSDGRRLVDGMSSW |
| | protein with MTP (SEQ | WAAIHGYNHPQLNAAMKSQIDAMSHVMFGGITHAPAIELCRKLVAMTPQPLECVFLADSGSVAVEVAMKMALQYWQAKGEARQRFLTFRNG |
| | ID NO: 201) | YHGDTFGAMSVCDPDNSMHSLWKGYLPENLFAPAPQSRMDGEWDERDMVGFARLMAAHRHEIAAVIIEPIVQGAGGMRMYHPEWLKRIRKI |
| | | CDREGILLIADEIATGFGRTGKLFACEHAEIAPDILCLGKALTGGTMTLSATLTTREVAETISNGEAGCFMHGPTFMGNPLACAAANASLAILES |
| | | GDWQQQVADIEVQLREQLAPARDAEMVADVRVLGAIGVVETTHPVNMAALQKFFVEQGVWIRPFGKLIYLMPPYIILPQQLQRLTAAVNRAV |
| | | QDETFFCQ |
| 202 | Escherichia coli BioA | ATGACTACTGATGATCTTGCTTTTGATCAAAGACATATTTGGCATCCATATACTTCTATGACTTCTCCACTTCCTGTTTATCCTGTTGTTTC |
| | nucleic acid (SEQ ID | TGCTGAAGGATGTGAACTTATTCTTTCTGATGGAAGAAGACTTGTTGATGGAATGTCTTCTTGGTGGGCTGCTATTCATGGATATAATCA |
| | NO: 202) | TCCACAACTTAATGCTGCTATGAAGTCTCAAATTGATGCTATGTCTCATGTTATGTTTGGAGGAATTACTCATGCTCCTGCTATTGAACTT |
| | | TGTAGAAAGCTTGTTGCTATGACTCCACAACCACTTGAATGTGTTTTTCTTGCTGATTCTGGATCTGTTGCTGTTGAAGTTGCTATGAAG |
| | | ATGGCTCTTCAATATTGGCAAGCTAAGGGAGAAGCTAGACAAAGATTTCTTACTTTTAGAAATGGATATCATGGAGATACTTTTGGAGCT |
| | | ATGTCTGTTTGTGATCCTGATAATTCTATGCATTCTCTTTGGAAGGGATATCTTCCTGAAAATCTTTTTGCTCCTGCTCCACAATCTAGAA |
| | | TGGATGGAGAATGGGATGAAAGAGATATGGTTGGATTTGCTAGACTTATGGCTGCTCATAGACATGAAATTGCTGCTGTTATTATTGAAC |
| | | CAATTGTTCAAGGAGCTGGAGGAATGAGAATGTATCATCCTGAATGGCTTAAGAGAATTAGAAAGATTTGTGATAGAGAAGGAATTCTTC |
| | | TTATTGCTGATGAAATTGCTACTGGATTTGGAAGAACTGGAAAGCTTTTTGCTTGTGAACATGCTGAAATTGCTCCTGATATTCTTTGTCT |
| | | TGGAAAGGCTCTTACTGGAGGAACTATGACTCTTTCTGCTACTCTTACTACTAGAGAAGTTGCTGAAACTATTTCTAATGGAGAAGCTGG |
| | | ATGTTTTATGCATGGACCAACTTTTATGGGAAATCCACTTGCTTGTGCTGCTGCTAATGCTTCTCTTGCTATTCTTGAATCTGGAGATTGG |
| | | CAACAACAAGTTGCTGATATTGAAGTTCAACTTAGAGAACAACTTGCTCCTGCTAGAGATGCTGAAATGGTTGCTGATGTTAGAGTTCTT |
| | | GGAGCTATTGGAGTTGTTGAAACTACTCATCCTGTTAATATGGCTGCTCTTCAAAAGTTTTTTGTTGAACAAGGAGTTTGGATTAGACCA |
| | | TTTGGAAAGCTTATTTATCTTATGCCACCATATATTATTCTTCCACAACAACTTCAAAGACTTACTGCTGCTGTTAATAGAGCTGTTCAAG |
| | | ATGAAACTTTTTTTTGTCAATAA |
| 203 | Escherichia coli BioA | ATGTCTGTTTCTTGTTGTTGTAGAAATCTTGGAAAGACTATTAAGAAGGCTATTCCATCTCATCATCTTCATCTTAGATCTCTTGGAGGAT |
| | nucleic acid with MTP | CTCTTTATAGAAGAATTCAATCTTCTTCTATGACTACTGATGATCTTGCTTTTGATCAAAGACATATTTGGCATCCATATACTTCTATGACT |
| | (SEQ ID NO: 203) | TCTCCACTTCCTGTTTATCCTGTTGTTTCTGCTGAAGGATGTGAACTTATTCTTTCTGATGGAAGAAGACTTGTTGATGGAATGTCTTCTT |
| | | GGTGGGCTGCTATTCATGGATATAATCATCCACAACTTAATGCTGCTATGAAGTCTCAAATTGATGCTATGTCTCATGTTATGTTTGGAG |
| | | GAATTACTCATGCTCCTGCTATTGAACTTTGTAGAAAGCTTGTTGCTATGACTCCACAACCACTTGAATGTGTTTTTCTTGCTGATTCTGG |
| | | ATCTGTTGCTGTTGAAGTTGCTATGAAGATGGCTCTTCAATATTGGCAAGCTAAGGGAGAAGCTAGACAAAGATTTCTTACTTTTAGAAA |
| | | TGGATATCATGGAGATACTTTTGGAGCTATGTCTGTTTGTGATCCTGATAATTCTATGCATTCTCTTTGGAAGGGATATCTTCCTGAAAAT |
| | | CTTTTTGCTCCTGCTCCACAATCTAGAATGGATGGAGAATGGGATGAAAGAGATATGGTTGGATTTGCTAGACTTATGGCTGCTCATAG |
| | | ACATGAAATTGCTGCTGTTATTATTGAACCAATTGTTCAAGGAGCTGGAGGAATGAGAATGTATCATCCTGAATGGCTTAAGAGAATTAG |
| | | AAAGATTTGTGATAGAGAAGGAATTCTTCTTATTGCTGATGAAATTGCTACTGGATTTGGAAGAACTGGAAAGCTTTTTGCTTGTGAACAT |
| | | GCTGAAATTGCTCCTGATATTCTTTGTCTTGGAAAGGCTCTTACTGGAGGAACTATGACTCTTTCTGCTACTCTTACTACTAGAGAAGTT |
| | | GCTGAAACTATTTCTAATGGAGAAGCTGGATGTTTTATGCATGGACCAACTTTTATGGGAAATCCACTTGCTTGTGCTGCTGCTAATGCT |
| | | TCTCTTGCTATTCTTGAATCTGGAGATTGGCAACAACAAGTTGCTGATATTGAAGTTCAACTTAGAGAACAACTTGCTCCTGCTAGAGAT |
| | | GCTGAAATGGTTGCTGATGTTAGAGTTCTTGGAGCTATTGGAGTTGTTGAAACTACTCATCCTGTTAATATGGCTGCTCTTCAAAAGTTT |
| | | TTTGTTGAACAAGGAGTTTGGATTAGACCATTTGGAAAGCTTATTTATCTTATGCCACCATATATTATTCTTCCACAACAACTTCAAAGAC |
| | | TTACTGCTGCTGTTAATAGAGCTGTTCAAGATGAAACTTTTTTTTGTCAATAA |
| | VECTOR SEQUENCES | |
| 204 | Cpf1 Maize expression | AACCCTGTGGTTGGCATGCACATACAAATGGACGAACGGATAAACCTTTTCACGCCCTTTTAAATATCCGATTATTCTAATAAACGCTCT |
| | vector (seq id no: 204) | TTTCTCTTAGGTTTACCCGCCAATATATCCTGTCAAACACTGATAGTTTAAACTGGCACTAGCCTAACGGTGTTGACTAACTAGGCCGCT |
| | | TCCCTAATTAGCTAAGGTACCGGCGCGCCACCGGTGAGCTCGGGACCCGAATTCATTATGTGGTCTAGGTAGGTTCTATATATAAGAAA |
| | | ACTTGAAATGTTCTAAAAAAAAATTCAAGCCCATGCATGATTGAAGCAAACGGTATAGCAACGGTGTTAACCTGATCTAGTGATCTCTTG |
| | | CAATCCTTAACGGCCACCTACCGCAGGTAGCAAACGGCGTCCCCCTCCTCGATATCTCCGCGGCGACCTCTGGCTTTTTCCGCGGAAT |
| | | TGCGCGGTGGGGACGGATTCCACGAGACCGCGACGCAACCGCCTCTCGCCGCTGGGCCCCACACCGCTCGGTGCCGTAGCCTCACG |
| | | GGACTCTTTCTCCCTCCTCCCCCGTTATAAATTGGCTTCATCCCCTCCTTGCCTCATCCATCCAAATCCCAGTCCCCAATCCCATCCCTT |
| | | CGTAGGAGAAATTCATCGAAGCTAAGCGAATCCTCGCGATCCTCTCAAGGTACTGCGAGTTTTCGATCCCCCTCTCGACCCCTCGTATG |
| | | TTTGTGTTTGTCGTAGCGTTTGATTAGGTATGCTTTCCCTGTTTGTGTTCGTCGTAGCGTTTGATTAGGTATGCTTTCCCTGTTCGTGTTC |
| | | ATCGTAGTGTTTGATTAGGTCGTGTGAGGCGATGGCCTGCTCGCGTCCTTCGATCTGTAGTCGATTTGCGGGTCGTGGTGTAGATCTG |
| | | CGGGCTGTGATGAAGTTATTTGGTGTGATCTGCTCGCCTGATTCTGCGGGTTGGCTCGAGTAGATATGATGGTTGGACCGGTTGGTTC |
| | | GTTTACCGCGCTAGGGTTGGGCTGGGATGATGTTGCATGCGCCGTTGCGCGTGATCCCGCAGCAGGACTTGCGTTTGATTGCCAGAT |
| | | CTCGTTACGATTATGTGATTTGGTTTGGACTTTTTAGATCTGTAGCTTCTGCTTATGTGCCAGATGCGCCTACTGCTCATATGCCTGATG |
| | | ATAATCATAAATGGCTGTGGAACTAACTAGTTGATTGCGGAGTCATGTATCAGCTACAGGTGTAGGGACTAGCTACAGGTGTAGGGACT |
| | | TGCGTCTAATTGTTTGGTCCTTTACTCATGTTGCAATTATGCAATTTAGTTTAGATTGTTTGTTCCACTCATCTAGGCTGTAAAAGGGACA |
| | | CTGCTTAGATTGCTGTTTAATCTTTTTAGTAGATTATATTATATTGGTAACTTATTACCCCTATTACATGCCATACGTGACTTCTGCTCATG |
| | | CCTGATGATAATCATAGATCACTGTGGAATTAATTAGTTGATTGTTGAATCATGTTTCATGTACATACCACGGCACAATTGCTTAGTTCCT |
| | | TAACAAATGCAAATTTTACTGATCCATGTATGATTTGCGTGGTTCTCTAATGTGAAATACTATAGCTACTTGTTAGTAAGAATCAGGTTCG |
| | | TATGCTTAATGCTGTATGTGCCTTCTGCTCATGCCTGATGATAATCATATATCACTGGAATTAATTAGTTGATCGTTTAATCATATATCAAG |
| | | TACATACCATGCCACAATTTTTAGTCACTTAACCCATGCAGATTGAACTGGTCCCTGCATGTTTTGCTAAATTGTTCTATTCTGATTAGAC |
| | | CATATATCATGTATTTTTTTTTGGTAATGGTTCTCTTATTTTAAATGCTATATAGTTCTGGTACTTGTTAGAAAGATCTGCTTCATAGTTTAG |
| | | TTGCCTATCCCTCGAATTAGGATGCTGAGCAGCTGATCCTATAGCTTTGTTTCATGTATCAATTCTTTTGTGTTCAACAGTCAGTTTTTGT |
| | | TAGATTCATTGTAACTTATGGTCGCTTACTCTTCTGGTCCTCAATGCTTGCAGGGATCCCCTAAATAGACCATGGCTCCTAAGAAGAAGC |
| | | GGAAGGTTGGTATTCACGGGGTGCCTGCGGCTTCAAAGCTCGAGAAATTCACCAACTGTTATTCGTTGAGCAAAACACTGCGGTTTAAA |
| | | GCGATTCCAGTCGGCAAGACTCAAGAGAATATAGACAATAAGCGGCTGTTGGTGGAAGATGAAAAGCGCGCGGAAGACTACAAAGGG |
| | | GTGAAGAAGTTGTTGGACAGATACTACCTCTCTTTTATCAATGATGTCTTGCACTCAATCAAATTGAAGAATCTGAACAACTACATCTCCC |
| | | TCTTCAGAAAGAAAACAAGGACAGAAAAGGAGAATAAGGAACTTGAAAATTTGGAGATCAATCTGAGGAAAGAGATCGCGAAAGCCTTT |
| | | AAAGGCAACGAAGGATACAAAAGTCTGTTCAAGAAGGATATAATTGAGACAATTTTGCCAGAGTTCCTCGATGACAAGGACGAGATTGC |
| | | GCTGGTCAATTCGTTCAACGGATTCACAACAGCATTCACAGGCTTCTTTGATAATCGGGAAAATATGTTCTCTGAGGAGGCAAAGTCCA |
| | | CTTCTATTGCGTTCAGGTGTATCAATGAGAATCTCACTAGGTACATTTCCAACATGGATATCTTTGAGAAGGTTGACGCAATTTTTGACAA |
| | | GCACGAAGTTCAGGAGATTAAGGAGAAGATCCTCAATTCCGATTATGACGTTGAGGACTTCTTCGAGGGTGAGTTTTTTAATTTCGTGCT |
| | | CACTCAAGAGGGTATCGACGTGTATAATGCGATCATCGGTGGGTTCGTGACTGAGTCCGGTGAAAAGATTAAGGGATTGAACGAGTAT |
| | | ATCAACCTTTACAACCAAAAGACGAAACAGAAGCTGCCAAAGTTCAAGCCTCTTTACAAACAGGTTCTTTCAGACCGCGAGTCACTCTC |
| | | GTTCTATGGGGAGGGCTACACTTCGGATGAGGAAGTCCTGGAGGTGTTCAGGAATACTCTCAATAAGAATTCGGAGATTTTCTCTTCTA |
| | | TAAAAAAACTGGAAAAGTTGTTTAAGAATTTTGACGAATACTCTAGCGCCGGCATATTTGTGAAAAACGGCCCGGCCATATCAACGATAA |
| | | GTAAAGATATCTTCGGCGAATGGAACGTGATCAGAGACAAATGGAACGCGGAGTATGACGATATTCACCTGAAGAAGAAGGCTGTCGT |
| | | AACGGAGAAGTACGAGGATGATCGCAGGAAAAGCTTCAAAAAGATCGGAAGTTTCAGCCTGGAACAGTTGCAGGAGTATGCTGACGCC |
| | | GATCTTAGCGTCGTCGAGAAGTTGAAGGAGATAATCATCCAAAAGGTCGACGAGATATATAAAGTCTATGGATCAAGTGAAAAACTGTT |
| | | CGACGCCGACTTCGTTTTGGAGAAGTCCCTGAAGAAGAACGACGCTGTTGTTGCCATTATGAAGGATCTGCTCGACAGCGTGAAGAGT |
| | | TTCGAGAACTATATTAAGGCTTTTTTCGGGGAGGGGAAGGAGACTAACAGAGATGAGTCCTTCTACGGAGACTTCGTCCTCGCGTACG |
| | | ATATACTCCTTAAGGTAGACCACATCTACGACGCAATCAGAAATTACGTGACACAAAAGCCGTACAGCAAGGACAAGTTCAAACTCTACT |
| | | TCCAGAACCCCCAGTTCATGGGCGGCTGGGACAAGGACAAGGAAACGGATTACAGGGCTACGATCCTGAGGTATGGTTCAAAATACTA |
| | | CTTGGCGATTATGGACAAGAAGTACGCCAAGTGTCTCCAGAAGATTGACAAAGACGATGTCAATGGCAATTATGAGAAGATCAACTACA |
| | | AGCTGCTTCCGGGTCCGAACAAGATGCTCCCAAAGGTTTTCTTCAGCAAGAAATGGATGGCCTACTATAACCCAAGCGAGGACATCCA |
| | | GAAGATTTATAAGAACGGTACGTTCAAGAAGGGCGACATGTTCAATCTTAACGACTGTCACAAGCTGATCGACTTCTTCAAAGACTCAAT |
| | | TAGCCGGTACCCAAAGTGGTCTAACGCCTATGACTTCAACTTTTCGGAAACCGAGAAGTACAAGGATATAGCCGGATTTTATAGAGAGG |
| | | TGGAAGAGCAGGGCTACAAGGTGTCATTCGAGTCCGCCAGCAAGAAGGAAGTGGACAAGCTCGTGGAAGAGGGTAAGCTCTACATGT |
| | | TCCAGATTTATAATAAAGACTTTAGCGATAAGAGCCACGGGACACCTAATCTCCACACAATGTATTTCAAGCTGCTCTTCGACGAGAATA |
| | | ACCACGGCCAAATCAGGTTGTCAGGAGGGGCTGAACTCTTCATGCGGCGCGCTAGCCTTAAGAAGGAGGAGCTTGTAGTCCACCCTG |
| | | CGAATAGTCCAATTGCGAATAAGAACCCGGACAATCCTAAAAAGACTACAACATTGAGCTACGACGTGTACAAGGATAAGAGGTTTTCC |
| | | GAGGATCAGTACGAGCTCCACATCCCGATTGCGATCAACAAGTGCCCAAAGAATATTTTCAAGATAAACACAGAGGTGCGTGTACTCCT |
| | | GAAGCATGACGACAATCCTTACGTCATTGGGATTGATCGGGGCGAGAGGAACCTCCTCTATATTGTGGTGGTGGACGGGAAGGGGAA |
| | | CATAGTCGAACAGTACTCCCTTAACGAAATAATTAACAATTTCAACGGCATCCGTATCAAGACCGACTACCATTCGTTGCTGGACAAGAA |
| | | GGAGAAGGAGAGATTTGAGGCGCGGCAAAATTGGACAAGTATCGAGAACATCAAGGAACTCAAAGCAGGTTATATCTCTCAAGTTGTG |
| | | CATAAGATATGCGAGCTGGTTGAGAAGTATGACGCAGTGATCGCTCTTGAGGACCTCAACTCGGGCTTTAAGAATTCTAGAGTTAAAGT |
| | | GGAGAAGCAGGTCTATCAAAAGTTCGAGAAGATGCTTATAGATAAGCTCAACTACATGGTCGATAAGAAATCGAACCCATGTGCCACCG |
| | | GCGGCGCACTCAAAGGTTACCAAATAACAAACAAATTCGAGTCCTTCAAATCGATGAGTACTCAGAATGGGTTCATATTTTATATACCGG |
| | | CGTGGCTTACGTCTAAGATCGACCCGTCAACTGGTTTTGTCAACCTGTTGAAGACGAAATACACGTCCATTGCCGATTCAAAAAAGTTC |
| | | ATATCTAGTTTTGATCGTATTATGTACGTCCCAGAGGAAGATCTTTTCGAGTTTGCTCTCGACTACAAAAACTTTTCGCGCACCGATGCG |
| | | GATTACATTAAAAAATGGAAACTCTATTCGTACGGCAACAGAATCAGGATTTTTCGCAACCCTAAGAAGAATAACGTCTTTGATTGGGAG |
| | | GAAGTTTGCTTGACTAGCGCGTACAAGGAGCTCTTTAATAAGTATGGCATTAACTACCAACAGGGTGATATCAGAGCACTGCTTTGCGA |
| | | ACAATCTGACAAGGCTTTCTACTCATCCTTCATGGCTTTGATGAGCCTGATGCTCCAGATGAGAAATTCAATTACAGGCAGAACCGACG |
| | | TGGATTTCTTGATCTCCCCGGTTAAAAATTCTGATGGCATCTTTTACGATAGCAGGAACTATGAAGCGCAAGAGAATGCGATTCTGCCAA |
| | | AAAATGCAGACGCCAACGGTGCCTATAACATCGCCAGGAAAGTCCTGTGGGCGATCGGCCAGTTCAAAAAGGCCGAAGACGAAAAATT |
| | | GGACAAGGTCAAAATCGCTATCAGCAACAAAGAGTGGCTGGAGTATGCTCAGACATCCGTAAAGCATAAGCGTCCTGCTGCCACCAAA |
| | | AAGGCCGGACAGGCTAAGAAAAAGAAGTGATTAATTAAGATCGTTCAAACATTTGGCAATAAAGTTTCTTAAGATTGAATCCTGTTGCCG |
| | | GTCTTGCGATGATTATCATATAATTTCTGTTGAATTACGTTAAGCATGTAATAATTAACATGTAATGCATGACGTTATTTATGAGATGGGTT |
| | | TTTATGATTAGAGTCCCGCAATTATACATTTAATACGCGATAGAAAACAAAATATAGCGCGCAAACTAGGATAAATTATCGCGCGCGGTG |
| | | TCATCTATGTTACTAGATCTTCGAAGAATTCATTATGTGGTCTAGGTAGGTTCTATATATAAGAAAACTTGAAATGTTCTAAAAAAAAATTC |
| | | AAGCCCATGCATGATTGAAGCAAACGGTATAGCAACGGTGTTAACCTGATCTAGTGATCTCTTGCAATCCTTAACGGCCACCTACCGCA |
| | | GGTAGCAAACGGCGTCCCCCTCCTCGATATCTCCGCGGCGACCTCTGGCTTTTTCCGCGGAATTGCGCGGTGGGGACGGATTCCACG |
| | | AGACCGCGACGCAACCGCCTCTCGCCGCTGGGCCCCACACCGCTCGGTGCCGTAGCCTCACGGGACTCTTTCTCCCTCCTCCCCCGT |
| | | TATAAATTGGCTTCATCCCCTCCTTGCCTCATCCATCCAAATCCCAGTCCCCAATCCCATCCCTTCGTAGGAGAAATTCATCGAAGCTAA |
| | | GCGAATCCTCGCGATCCTCTCAAGGTACTGCGAGTTTTCGATCCCCCTCTCGACCCCTCGTATGTTTGTGTTTGTCGTAGCGTTTGATT |
| | | AGGTATGCTTTCCCTGTTTGTGTTCGTCGTAGCGTTTGATTAGGTATGCTTTCCCTGTTCGTGTTCATCGTAGTGTTTGATTAGGTCGTG |
| | | TGAGGCGATGGCCTGCTCGCGTCCTTCGATCTGTAGTCGATTTGCGGGTCGTGGTGTAGATCTGCGGGCTGTGATGAAGTTATTTGGT |
| | | GTGATCTGCTCGCCTGATTCTGCGGGTTGGCTCGAGTAGATATGATGGTTGGACCGGTTGGTTCGTTTACCGCGCTAGGGTTGGGCTG |
| | | GGATGATGTTGCATGCGCCGTTGCGCGTGATCCCGCAGCAGGACTTGCGTTTGATTGCCAGATCTCGTTACGATTATGTGATTTGGTTT |
| | | GGACTTTTTAGATCTGTAGCTTCTGCTTATGTGCCAGATGCGCCTACTGCTCATATGCCTGATGATAATCATAAATGGCTGTGGAACTAA |
| | | CTAGTTGATTGCGGAGTCATGTATCAGCTACAGGTGTAGGGACTAGCTACAGGTGTAGGGACTTGCGTCTAATTGTTTGGTCCTTTACT |
| | | CATGTTGCAATTATGCAATTTAGTTTAGATTGTTTGTTCCACTCATCTAGGCTGTAAAAGGGACACTGCTTAGATTGCTGTTTAATCTTTTT |
| | | AGTAGATTATATTATATTGGTAACTTATTACCCCTATTACATGCCATACGTGACTTCTGCTCATGCCTGATGATAATCATAGATCACTGTG |
| | | GAATTAATTAGTTGATTGTTGAATCATGTTTCATGTACATACCACGGCACAATTGCTTAGTTCCTTAACAAATGCAAATTTTACTGATCCAT |
| | | GTATGATTTGCGTGGTTCTCTAATGTGAAATACTATAGCTACTTGTTAGTAAGAATCAGGTTCGTATGCTTAATGCTGTATGTGCCTTCTG |
| | | CTCATGCCTGATGATAATCATATATCACTGGAATTAATTAGTTGATCGTTTAATCATATATCAAGTACATACCATGCCACAATTTTTAGTCA |
| | | CTTAACCCATGCAGATTGAACTGGTCCCTGCATGTTTTGCTAAATTGTTCTATTCTGATTAGACCATATATCATGTATTTTTTTTTGGTAAT |
| | | GGTTCTCTTATTTTAAATGCTATATAGTTCTGGTACTTGTTAGAAAGATCTGCTTCATAGTTTAGTTGCCTATCCCTCGAATTAGGATGCT |
| | | GAGCAGCTGATCCTATAGCTTTGTTTCATGTATCAATTCTTTTGTGTTCAACAGTCAGTTTTTGTTAGATTCATTGTAACTTATGGTCGCTT |
| | | ACTCTTCTGGTCCTCAATGCTTGCAGGGGATCCAAATTACTGATGAGTCCGTGAGGACGAAACGAGTAAGCTCGTCTAATTTCTACTAA |
| | | GTGTAGATATGCATGCGCAAGTTGGTGGACTGGCCGGCATGGTCCCAGCCTCCTCGCTGGCGCCGGCTGGGCAACATGCTTCGGCAT |
| | | GGCGAATGGGACGATCGTTCAAACATTTGGCAATAAAGTTTCTTAAGATTGAATCCTGTTGCCGGTCTTGCGATGATTATCATATAATTT |
| | | CTGTTGAATTACGTTAAGCATGTAATAATTAACATGTAATGCATGACGTTATTTATGAGATGGGTTTTTATGATTAGAGTCCCGCAATTAT |
| | | ACATTTAATACGCGATAGAAAACAAAATATAGCGCGCAAACTAGGATAAATTATCGCGCGCGGTGTCATCTATGTTACTAGATCCGGAC |
| | | CGCGCCTGCAGTGCAGCGTGACCCGGTCGTGCCCCTCTCTAGAGATAATGAGCATTGCATGTCTAAGTTATAAAAAATTACCACATATT |
| | | TTTTTTGTCACACTTGTTTGAAGTGCAGTTTATCTATCTTTATACATATATTTAAACTTTACTCTACGAATAATATAATCTATAGTACTACAA |
| | | TAATATCAGTGTTTTAGAGAATCATATAAATGAACAGTTAGACATGGTCTAAAGGACAATTGAGTATTTTGACAACAGGACTCTACAGTTT |
| | | TATCTTTTTAGTGTGCATGTGTTCTCCTTTTTTTTTGCAAATAGCTTCACCTATATAATACTTCATCCATTTTATTAGTACATCCATTTAGGG |
| | | TTTAGGGTTAATGGTTTTTATAGACTAATTTTTTTAGTACATCTATTTTATTCTATTTTAGCCTCTAAATTAAGAAAACTAAAACTCTATTTTA |
| | | GTTTTTTTATTTAATAATTTAGATATAAAATAGAATAAAATAAAGTGACTAAAAATTAAACAAATACCCTTTAAGAAATTAAAAAAACTAAGG |
| | | AAACATTTTTCTTGTTTCGAGTAGATAATGCCAGCCTGTTAAACGCCGTCGACGAGTCTAACGGACACCAACCAGCGAACCAGCAGCGT |
| | | CGCGTCGGGCCAAGCGAAGCAGACGGCACGGCATCTCTGTCGCTGCCTCTGGACCCCTCTCGAGAGTTCCGCTCCACCGTTGGACTT |
| | | GCTCCGCTGTCGGCATCCAGAAATTGCGTGGCGGAGCGGCAGACGTGAGCCGGCACGGCAGGCGGCCTCCTCCTCCTCTCACGGCA |
| | | CCGGCAGCTACGGGGGATTCCTTTCCCACCGCTCCTTCGCTTTCCCTTCCTCGCCCGCCGTAATAAATAGACACCCCCTCCACACCCT |
| | | CTTTCCCCAACCTCGTGTTGTTCGGAGCGCACACACACACAACCAGATCTCCCCCAAATCCACCCGTCGGCACCTCCGCTTCAAGGTA |
| | | CGCCGCTCGTCCTCCCCCCCCCCCCTCTCTACCTTCTCTAGATCGGCGTTCCGGTCCATGGTTAGGGCCCGGTAGTTCTACTTCTGTT |
| | | CATGTTTGTGTTAGATCCGTGTTTGTGTTAGATCCGTGCTGCTAGCGTTCGTACACGGATGCGACCTGTACGTCAGACACGTTCTGATT |
| | | GCTAACTTGCCAGTGTTTCTCTTTGGGGAATCCTGGGATGGCTCTAGCCGTTCCGCAGACGGGATCGATTTCATGATTTTTTTTGTTTCG |
| | | TTGCATAGGGTTTGGTTTGCCCTTTTCCTTTATTTCAATATATGCCGTGCACTTGTTTGTCGGGTCATCTTTTCATGCTTTTTTTTGTCTTG |
| | | GTTGTGATGATGTGGTCTGGTTGGGGGGTCGTTCTAGATCGGAGTAGAATTCTGTTTCAAACTACCTGGTGGATTTATTAATTTTGGATC |
| | | TGTATGTGTGTGCCATACATATTCATAGTTACGAATTGAAGATGATGGATGGAAATATCGATCTAGGATAGGTATACATGTTGATGCGGG |
| | | TTTTACTGATGCATATACAGAGATGCTTTTTGTTCGCTTGGTTGTGATGATGTGGTGTGGTTGGGGGGTCGTTCATTCGTTCTAGATCGG |
| | | AGTAGAATACTGTTTCAAACTACCTGGTGTATTTATTAATTTTGGAACTGTATGTGTGTGTCATACATCTTCATAGTTACGAGTTTAAGAT |
| | | GGATGGAAATATCGATCTAGGATAGGTATACATGTTGATGTGGGTTTTACTGATGCATATACATGATGGCATATGCAGCATCTATTCATA |
| | | TGCTCTAACCTTGAGTACCTATCTATTATAATAAACAAGTATGTTTTATAATTATTTTGATCTTGATATACTTGGATGATGGCATATGCAGC |
| | | AGCTATATGTGGATTTTTTTAGCCCTGCCTTCATACGCTATTTATTTGCTTGGTACTGTTTCTTTTGTCGATGCTCACCCTGTTGTTTGGT |
| | | GTTACTTCTGCAGGGATCCGGCAGCAGCCATGCAGAAGCTGATCAACAGCGTGCAGAACTACGCCTGGGGCAGCAAGACCGCCCTGA |
| | | CCGAGCTGTACGGCATGGAGAACCCCAGCAGCCAGCCCATGGCCGAGCTGTGGATGGGCGCCCACCCCAAGAGCAGCAGCCGCGTG |
| | | CAGAACGCCGCCGGCGACATCGTGAGCCTGCGCGACGTGATCGAGAGCGACAAGAGCACCCTGCTGGGCGAGGCCGTGGCCAAGC |
| | | GCTTCGGCGAGCTGCCCTTCCTGTTCAAGGTGCTGTGCGCCGCCCAGCCCCTGAGCATCCAGGTGCACCCCAACAAGCACAACAGCG |
| | | AGATCGGCTTCGCCAAGGAGAACGCCGCCGGCATCCCCATGGACGCCGCCGAGCGCAACTACAAGGACCCCAACCACAAGCCCGAG |
| | | CTGGTGTTCGCCCTGACCCCCTTCCTGGCCATGAACGCCTTCCGCGAGTTCAGCGAGATCGTGAGCCTGCTGCAGCCCGTGGCCGGC |
| | | GCCCACCCCGCCATCGCCCACTTCCTGCAGCAGCCCGACGCCGAGCGCCTGAGCGAGCTGTTCGCCAGCCTGCTGAACATGCAGGG |
| | | CGAGGAGAAGAGCCGCGCCCTGGCCATCCTGAAGAGCGCCCTGGACAGCCAGCAGGGCGAGCCCTGGCAGACCATCCGCCTGATCA |
| | | GCGAGTTCTACCCCGAGGACAGCGGCCTGTTCAGCCCCCTGCTGCTGAACGTGGTGAAGCTGAACCCCGGCGAGGCCATGTTCCTGT |
| | | TCGCCGAGACCCCCCACGCCTACCTGCAGGGCGTGGCCCTGGAGGTGATGGCCAACAGCGACAACGTGCTGCGCGCCGGCCTGACC |
| | | CCCAAGTACATCGACATCCCCGAGCTGGTGGCCAACGTGAAGTTCGAGGCCAAGCCCGCCAACCAGCTGCTGACCCAGCCCGTGAAG |
| | | CAGGGCGCCGAGCTGGACTTCCCCATCCCCGTGGACGACTTCGCCTTCAGCCTGCACGACCTGAGCGACAAGGAGACCACCATCAGC |
| | | CAGCAGAGCGCCGCCATCCTGTTCTGCGTGGAGGGCGACGCCACCCTGTGGAAGGGCAGCCAGCAGCTGCAGCTGAAGCCCGGCGA |
| | | GAGCGCCTTCATCGCCGCCAACGAGAGCCCCGTGACCGTGAAGGGCCACGGCCGCCTGGCCCGCGTGTACAACAAGCTGTGATAGG |
| | | AGCTCGATCCGTCGACCTGCAGATCGTTCAAACATTTGGCAATAAAGTTTCTTAAGATTGAATCCTGTTGCCGGTCTTGCGATGATTATC |
| | | ATATAATTTCTGTTGAATTACGTTAAGCATGTAATAATTAACATGTAATGCATGACGTTATTTATGAGATGGGTTTTTATGATTAGAGTCCC |
| | | GCAATTATACATTTAATACGCGATAGAAAACAAAATATAGCGCGCAAACTAGGATAAATTATCGCGCGCGGTGTCATCTATGTTACTAGA |
| | | TCGGCGCGCCGCAATTGAAGTTTGGGCGGCCAGCATGGCCGTATCCGCAATGTGTTATTAAGTTGTCTAAGCGTCAATTTGTTTACACC |
| | | ACAATATATCCTGCCACCAGCCAGCCAACAGCTCCCCGACCGGCAGCTCGGCACAAAATCACCACTCGATACAGGCAGCCCATCAGAA |
| | | TTAATTCTCATGTTTGACAGCTTATCATCGACTGCACGGTGCACCAATGCTTCTGGCGTCAGGCAGCCATCGGAAGCTGTGGTATGGCT |
| | | GTGCAGGTCGTAAATCACTGCATAATTCGTGTCGCTCAAGGCGCACTCCCGTTCTGGATAATGTTTTTTGCGCCGACATCATAACGGTT |
| | | CTGGCAAATATTCTGAAATGAGCTGTTGACAATTAATCATCCGGCTCGTATAATGTGTGGAATTGTGAGCGGATAACAATTTCACACAGG |
| | | AAACAGACCATGAGGGAAGCGTTGATCGCCGAAGTATCGACTCAACTATCAGAGGTAGTTGGCGTCATCGAGCGCCATCTCGAACCGA |
| | | CGTTGCTGGCCGTACATTTGTACGGCTCCGCAGTGGATGGCGGCCTGAAGCCACACAGTGATATTGATTTGCTGGTTACGGTGACCGT |
| | | AAGGCTTGATGAAACAACGCGGCGAGCTTTGATCAACGACCTTTTGGAAACTTCGGCTTCCCCTGGAGAGAGCGAGATTCTCCGCGCT |
| | | GTAGAAGTCACCATTGTTGTGCACGACGACATCATTCCGTGGCGTTATCCAGCTAAGCGCGAACTGCAATTTGGAGAATGGCAGCGCA |
| | | ATGACATTCTTGCAGGTATCTTCGAGCCAGCCACGATCGACATTGATCTGGCTATCTTGCTGACAAAAGCAAGAGAACATAGCGTTGCC |
| | | TTGGTAGGTCCAGCGGCGGAGGAACTCTTTGATCCGGTTCCTGAACAGGATCTATTTGAGGCGCTAAATGAAACCTTAACGCTATGGAA |
| | | CTCGCCGCCCGACTGGGCTGGCGATGAGCGAAATGTAGTGCTTACGTTGTCCCGCATTTGGTACAGCGCAGTAACCGGCAAAATCGC |
| | | GCCGAAGGATGTCGCTGCCGACTGGGCAATGGAGCGCCTGCCGGCCCAGTATCAGCCCGTCATACTTGAAGCTAGGCAGGCTTATCT |
| | | TGGACAAGAAGATCGCTTGGCCTCGCGCGCAGATCAGTTGGAAGAATTTGTTCACTACGTGAAAGGCGAGATCACCAAAGTAGTCGGC |
| | | AAATAAAGCTCTAGTGGATCTCCGTACCCGGGGATCTGGCTCGCGGCGGACGCACGACGCCGGGGCGAGACCATAGGCGATCTCCTA |
| | | AATCAATAGTAGCTGTAACCTCGAAGCGTTTCACTTGTAACAACGATTGAGAATTTTTGTCATAAAATTGAAATACTTGGTTCGCATTTTT |
| | | GTCATCCGCGGTCAGCCGCAATTCTGACGAACTGCCCATTTAGCTGGAGATGATTGTACATCCTTCACGTGAAAATTTCTCAAGCGCTG |
| | | TGAACAAGGGTTCAGATTTTAGATTGAAAGGTGAGCCGTTGAAACACGTTCTTCTTGTCGATGACGACGTCGCTATGCGGCATCTTATT |
| | | ATTGAATACCTTACGATCCACGCCTTCAAAGTGACCGCGGTAGCCGACAGCACCCAGTTCACAAGAGTACTCTCTTCCGCGACGGTCG |
| | | ATGTCGTGGTTGTTGATCTAGATTTAGGTCGTGAAGATGGGCTCGAGCTAGGAGCAAGTGATTTTATCGCTAAGCCGTTCAGTATCAGA |
| | | GAGTTTCTAGCACGCATTCGGGTTGCCTTGCGCGTGCGCCCCAACGTTGTCCGCTCCAAAGACCGACGGTCTTTTTGTTTTACTGACTG |
| | | GACACTTAATCTCAGGCAACGTCGCTTGATGTCCGAAGCTGGCGGTGAGGTGAAACTTACGGCAGGTGAGTTCAATCTTCTCCTCGCG |
| | | TTTTTAGAGAAACCCCGCGACGTTCTATCGCGCGAGCAACTTCTCATTGCCAGTCGAGTACGCGACGAGGAGGTTTATGACAGGAGTA |
| | | TAGATGTTCTCATTTTGAGGCTGCGCCGCAAACTTGAGGCAGATCCGTCAAGCCCTCAACTGATAAAAACAGCAAGAGGTGCCGGTTAT |
| | | TTCTTTGACGCGGACGTGCAGGTTTCGCACGGGGGGACGATGGCAGCCTGAGCCAATTCCCAGATCCCCGAGGAATCGGCGTGAGC |
| | | GGTCGCAAACCATCCGGCCCGGTACAAATCGGCGCGGCGCTGGGTGATGACCTGGTGGAGAAGTTGAAGGCCGCGCAGGCCGCCCA |
| | | GCGGCAACGCATCGAGGCAGAAGCACGCCCCGGTGAATCGTGGCAAGCGGCCGCTGATCGAATCCGCAAAGAATCCCGGCAACCGC |
| | | CGGCAGCCGGTGCGCCGTCGATTAGGAAGCCGCCCAAGGGCGACGAGCAACCAGATTTTTTCGTTCCGATGCTCTATGACGTGGGCA |
| | | CCCGCGATAGTCGCAGCATCATGGACGTGGCCGTTTTCCGTCTGTCGAAGCGTGACCGACGAGCTGGCGAGGTGATCCGCTACGAGC |
| | | TTCCAGACGGGCACGTAGAGGTTTCCGCAGGGCCGGCCGGCATGGCCAGTGTGTGGGATTACGACCTGGTACTGATGGCGGTTTCCC |
| | | ATCTAACCGAATCCATGAACCGATACCGGGAAGGGAAGGGAGACAAGCCCGGCCGCGTGTTCCGTCCACACGTTGCGGACGTACTCA |
| | | AGTTCTGCCGGCGAGCCGATGGCGGAAAGCAGAAAGACGACCTGGTAGAAACCTGCATTCGGTTAAACACCACGCACGTTGCCATGC |
| | | AGCGTACGAAGAAGGCCAAGAACGGCCGCCTGGTGACGGTATCCGAGGGTGAAGCCTTGATTAGCCGCTACAAGATCGTAAAGAGCG |
| | | AAACCGGGGGGCCGGAGTACATCGAGATCGAGCTAGCTGATTGGATGTACCGCGAGATCACAGAAGGCAAGAACCCGGACGTGCTGA |
| | | CGGTTCACCCCGATTACTTTTTGATCGATCCCGGCATCGGCCGTTTTCTCTACCGCCTGGCACGCCGCGCCGCAGGCAAGGCAGAAG |
| | | CCAGATGGTTGTTCAAGACGATCTACGAACGCAGTGGCAGCGCCGGAGAGTTCAAGAAGTTCTGTTTCACCGTGCGCAAGCTGATCGG |
| | | GTCAAATGACCTGCCGGAGTACGATTTGAAGGAGGAGGGGGGCAGGCTGGCCCGATCCTAGTCATGCGCTACCGCAACCTGATCGA |
| | | GGGCGAAGCATCCGCCGGTTCCTAATGTACGGAGCAGATGCTAGGGCAAATTGCCCTAGCAGGGGAAAAAGGTCGAAAAGGTCTCTT |
| | | TCCTGTGGATAGCACGTACATTGGGAACCCAAAGCCGTACATTGGGAACCGGAACCCGTACATTGGGAACCCAAAGCCGTACATTGGG |
| | | AACCGGTCACACATGTAAGTGACTGATATAAAAGAGAAAAAAGGCGATTTTTCCGCCTAAAACTCTTTAAAACTTATTAAAACTCTTAAAA |
| | | CCCGCCTGGCCTGTGCATAACTGTCTGGCCAGCGCACAGCCGAAGAGCTGCAAAAAGCGCCTACCCTTCGGTCGCTGCGCTCCCTAC |
| | | GCCCCGCCGCTTCGCGTCGGCCTATCGCGGCCGCTGGCCGCTCAAAAATGGCTGGCCTACGGCCAGGCAATCTACCAGGGCGCGGA |
| | | CAAGCCGCGCCGTCGCCACTCGACCGCCGGCGCTGAGGTCTGCCTCGTGAAGAAGGTGTTGCTGACTCATACCAGGCCTGAATCGCC |
| | | CCATCATCCAGCCAGAAAGTGAGGGAGCCACGGTTGATGAGAGCTTTGTTGTAGGTGGACCAGTTGGTGATTTTGAACTTTTGCTTTGC |
| | | CACGGAACGGTCTGCGTTGTCGGGAAGATGCGTGATCTGATCCTTCAACTCAGCAAAAGTTCGATTTATTCAACAAAGCCGCCGTCCC |
| | | GTCAAGTCAGCGTAATGCTCTGCCAGTGTTACAACCAATTAACCAATTCTGATTAGAAAAACTCATCGAGCATCAAATGAAACTGCAATT |
| | | TATTCATATCAGGATTATCAATACCATATTTTTGAAAAAGCCGTTTCTGTAATGAAGGAGAAAACTCACCGAGGCAGTTCCATAGGATGG |
| | | CAAGATCCTGGTATCGGTCTGCGATTCCGACTCGTCCAACATCAATACAACCTATTAATTTCCCCTCGTCAAAAATAAGGTTATCAAGTG |
| | | AGAAATCACCATGAGTGACGACTGAATCCGGTGAGAATGGCAAAAGCTCTGCATTAATGAATCGGCCAACGCGCGGGGAGAGGCGGT |
| | | TTGCGTATTGGGCGCTCTTCCGCTTCCTCGCTCACTGACTCGCTGCGCTCGGTCGTTCGGCTGCGGCGAGCGGTATCAGCTCACTCAA |
| | | AGGCGGTAATACGGTTATCCACAGAATCAGGGGATAACGCAGGAAAGAACATGTGAGCAAAAGGCCAGCAAAAGGCCAGGAACCGTA |
| | | AAAAGGCCGCGTTGCTGGCGTTTTTCCATAGGCTCCGCCCCCCTGACGAGCATCACAAAAATCGACGCTCAAGTCAGAGGTGGCGAAA |
| | | CCCGACAGGACTATAAAGATACCAGGCGTTTCCCCCTGGAAGCTCCCTCGTGCGCTCTCCTGTTCCGACCCTGCCGCTTACCGGATAC |
| | | CTGTCCGCCTTTCTCCCTTCGGGAAGCGTGGCGCTTTCTCATAGCTCACGCTGTAGGTATCTCAGTTCGGTGTAGGTCGTTCGCTCCAA |
| | | GCTGGGCTGTGTGCACGAACCCCCCGTTCAGCCCGACCGCTGCGCCTTATCCGGTAACTATCGTCTTGAGTCCAACCCGGTAAGACA |
| | | CGACTTATCGCCACTGGCAGCAGCCACTGGTAACAGGATTAGCAGAGCGAGGTATGTAGGCGGTGCTACAGAGTTCTTGAAGTGGTG |
| | | GCCTAACTACGGCTACACTAGAAGAACAGTATTTGGTATCTGCGCTCTGCTGAAGCCAGTTACCTTCGGAAAAAGAGTTGGTAGCTCTT |
| | | GATCCGGCAAACAAACCACCGCTGGTAGCGGTGGTTTTTTTGTTTGCAAGCAGCAGATTACGCGCAGAAAAAAAGGATCTCAAGAAGAT |
| | | CCTTTGATCTTTTCTACGGGGTCTGACGCTCAGTGGAACGAAAACTCACGTTAAGGGATTTTGGTCATGAGATTATCAAAAAGGATCTTC |
| | | ACCTAGATCCTTTTGATCCGGACAAACAAACAAATACAGTAATTTAGCCAGGACGTCGGCCGAAAGAGCGACAAGCAGATCACGCTTTT |
| | | CGACAGCGTCGGATTTGCGATCGAGGATTTTTCGGCGCTGCGCTACGTCCGCGACCGCGTTGAGGGATCAAGCCACAGCAGCCCACT |
| | | CGACCTTCTAGCCGACCCAGACGAGCCAAGGGATCTTTTTGGAATGCTGCTCCGTCGTCAGGCTTTCCGACGTTTGGGTGGTTGAACA |
| | | GAAGTCATTATCGCACGGAATGCCAAGCACTCCCGAGGGG |
| 205 | Donor sequence for corn | GATTCTCTAAATCTTTGATGTAGATTAAAGACAAAATGCTCATCCCTCAATTCGATGCAGATGCAAGTTGGTGGACTCAAGGGCCTGATT |
| | bioda c372d (Seq id | CTAACTTGCAGGTTTTGTCCTCACAGATCTGGATATG |
| | no: 205) | |
| 312 | CPF1 SOY | AACCCTGTGGTTGGCATGCACATACAAATGGACGAACGGATAAACCTTTTCACGCCCTTTTAAATATCCGATTATTCTAATAAACGCTCT |
| | EXPRESSION VECTOR | TTTCTCTTAGGTTTACCCGCCAATATATCCTGTCAAACACTGATAGTTTAAACTGGCACTAGCCTAACGGTGTTGACTAACTAGGCCGCT |
| | (SEQ ID NO: 312) | TCCCTAATTAGCTAAGGTACCGGCGCGCCACCGGTGAGCTCGGGACCCGAATTCATTATGTGGTCTAGGTAGGTTCTATATATAAGAAA |
| | | ACTTGAAATGTTCTAAAAAAAAATTCAAGCCCATGCATGATTGAAGCAAACGGTATAGCAACGGTGTTAACCTGATCTAGTGATCTCTTG |
| | | CAATCCTTAACGGCCACCTACCGCAGGTAGCAAACGGCGTCCCCCTCCTCGATATCTCCGCGGCGACCTCTGGCTTTTTCCGCGGAAT |
| | | TGCGCGGTGGGGACGGATTCCACGAGACCGCGACGCAACCGCCTCTCGCCGCTGGGCCCCACACCGCTCGGTGCCGTAGCCTCACG |
| | | GGACTCTTTCTCCCTCCTCCCCCGTTATAAATTGGCTTCATCCCCTCCTTGCCTCATCCATCCAAATCCCAGTCCCCAATCCCATCCCTT |
| | | CGTAGGAGAAATTCATCGAAGCTAAGCGAATCCTCGCGATCCTCTCAAGGTACTGCGAGTTTTCGATCCCCCTCTCGACCCCTCGTATG |
| | | TTTGTGTTTGTCGTAGCGTTTGATTAGGTATGCTTTCCCTGTTTGTGTTCGTCGTAGCGTTTGATTAGGTATGCTTTCCCTGTTCGTGTTC |
| | | ATCGTAGTGTTTGATTAGGTCGTGTGAGGCGATGGCCTGCTCGCGTCCTTCGATCTGTAGTCGATTTGCGGGTCGTGGTGTAGATCTG |
| | | CGGGCTGTGATGAAGTTATTTGGTGTGATCTGCTCGCCTGATTCTGCGGGTTGGCTCGAGTAGATATGATGGTTGGACCGGTTGGTTC |
| | | GTTTACCGCGCTAGGGTTGGGCTGGGATGATGTTGCATGCGCCGTTGCGCGTGATCCCGCAGCAGGACTTGCGTTTGATTGCCAGAT |
| | | CTCGTTACGATTATGTGATTTGGTTTGGACTTTTTAGATCTGTAGCTTCTGCTTATGTGCCAGATGCGCCTACTGCTCATATGCCTGATG |
| | | ATAATCATAAATGGCTGTGGAACTAACTAGTTGATTGCGGAGTCATGTATCAGCTACAGGTGTAGGGACTAGCTACAGGTGTAGGGACT |
| | | TGCGTCTAATTGTTTGGTCCTTTACTCATGTTGCAATTATGCAATTTAGTTTAGATTGTTTGTTCCACTCATCTAGGCTGTAAAAGGGACA |
| | | CTGCTTAGATTGCTGTTTAATCTTTTTAGTAGATTATATTATATTGGTAACTTATTACCCCTATTACATGCCATACGTGACTTCTGCTCATG |
| | | CCTGATGATAATCATAGATCACTGTGGAATTAATTAGTTGATTGTTGAATCATGTTTCATGTACATACCACGGCACAATTGCTTAGTTCCT |
| | | TAACAAATGCAAATTTTACTGATCCATGTATGATTTGCGTGGTTCTCTAATGTGAAATACTATAGCTACTTGTTAGTAAGAATCAGGTTCG |
| | | TATGCTTAATGCTGTATGTGCCTTCTGCTCATGCCTGATGATAATCATATATCACTGGAATTAATTAGTTGATCGTTTAATCATATATCAAG |
| | | TACATACCATGCCACAATTTTTAGTCACTTAACCCATGCAGATTGAACTGGTCCCTGCATGTTTTGCTAAATTGTTCTATTCTGATTAGAC |
| | | CATATATCATGTATTTTTTTTTGGTAATGGTTCTCTTATTTTAAATGCTATATAGTTCTGGTACTTGTTAGAAAGATCTGCTTCATAGTTTAG |
| | | TTGCCTATCCCTCGAATTAGGATGCTGAGCAGCTGATCCTATAGCTTTGTTTCATGTATCAATTCTTTTGTGTTCAACAGTCAGTTTTTGT |
| | | TAGATTCATTGTAACTTATGGTCGCTTACTCTTCTGGTCCTCAATGCTTGCAGGGATCCCCTAAATAGACCATGGCTCCTAAGAAGAAGC |
| | | GGAAGGTTGGTATTCACGGGGTGCCTGCGGCTTCAAAGCTCGAGAAATTCACCAACTGTTATTCGTTGAGCAAAACACTGCGGTTTAAA |
| | | GCGATTCCAGTCGGCAAGACTCAAGAGAATATAGACAATAAGCGGCTGTTGGTGGAAGATGAAAAGCGCGCGGAAGACTACAAAGGG |
| | | GTGAAGAAGTTGTTGGACAGATACTACCTCTCTTTTATCAATGATGTCTTGCACTCAATCAAATTGAAGAATCTGAACAACTACATCTCCC |
| | | TCTTCAGAAAGAAAACAAGGACAGAAAAGGAGAATAAGGAACTTGAAAATTTGGAGATCAATCTGAGGAAAGAGATCGCGAAAGCCTTT |
| | | AAAGGCAACGAAGGATACAAAAGTCTGTTCAAGAAGGATATAATTGAGACAATTTTGCCAGAGTTCCTCGATGACAAGGACGAGATTGC |
| | | GCTGGTCAATTCGTTCAACGGATTCACAACAGCATTCACAGGCTTCTTTGATAATCGGGAAAATATGTTCTCTGAGGAGGCAAAGTCCA |
| | | CTTCTATTGCGTTCAGGTGTATCAATGAGAATCTCACTAGGTACATTTCCAACATGGATATCTTTGAGAAGGTTGACGCAATTTTTGACAA |
| | | GCACGAAGTTCAGGAGATTAAGGAGAAGATCCTCAATTCCGATTATGACGTTGAGGACTTCTTCGAGGGTGAGTTTTTTAATTTCGTGCT |
| | | CACTCAAGAGGGTATCGACGTGTATAATGCGATCATCGGTGGGTTCGTGACTGAGTCCGGTGAAAAGATTAAGGGATTGAACGAGTAT |
| | | ATCAACCTTTACAACCAAAAGACGAAACAGAAGCTGCCAAAGTTCAAGCCTCTTTACAAACAGGTTCTTTCAGACCGCGAGTCACTCTC |
| | | GTTCTATGGGGAGGGCTACACTTCGGATGAGGAAGTCCTGGAGGTGTTCAGGAATACTCTCAATAAGAATTCGGAGATTTTCTCTTCTA |
| | | TAAAAAAACTGGAAAAGTTGTTTAAGAATTTTGACGAATACTCTAGCGCCGGCATATTTGTGAAAAACGGCCCGGCCATATCAACGATAA |
| | | GTAAAGATATCTTCGGCGAATGGAACGTGATCAGAGACAAATGGAACGCGGAGTATGACGATATTCACCTGAAGAAGAAGGCTGTCGT |
| | | AACGGAGAAGTACGAGGATGATCGCAGGAAAAGCTTCAAAAAGATCGGAAGTTTCAGCCTGGAACAGTTGCAGGAGTATGCTGACGCC |
| | | GATCTTAGCGTCGTCGAGAAGTTGAAGGAGATAATCATCCAAAAGGTCGACGAGATATATAAAGTCTATGGATCAAGTGAAAAACTGTT |
| | | CGACGCCGACTTCGTTTTGGAGAAGTCCCTGAAGAAGAACGACGCTGTTGTTGCCATTATGAAGGATCTGCTCGACAGCGTGAAGAGT |
| | | TTCGAGAACTATATTAAGGCTTTTTTCGGGGAGGGGAAGGAGACTAACAGAGATGAGTCCTTCTACGGAGACTTCGTCCTCGCGTACG |
| | | ATATACTCCTTAAGGTAGACCACATCTACGACGCAATCAGAAATTACGTGACACAAAAGCCGTACAGCAAGGACAAGTTCAAACTCTACT |
| | | TCCAGAACCCCCAGTTCATGGGCGGCTGGGACAAGGACAAGGAAACGGATTACAGGGCTACGATCCTGAGGTATGGTTCAAAATACTA |
| | | CTTGGCGATTATGGACAAGAAGTACGCCAAGTGTCTCCAGAAGATTGACAAAGACGATGTCAATGGCAATTATGAGAAGATCAACTACA |
| | | AGCTGCTTCCGGGTCCGAACAAGATGCTCCCAAAGGTTTTCTTCAGCAAGAAATGGATGGCCTACTATAACCCAAGCGAGGACATCCA |
| | | GAAGATTTATAAGAACGGTACGTTCAAGAAGGGCGACATGTTCAATCTTAACGACTGTCACAAGCTGATCGACTTCTTCAAAGACTCAAT |
| | | TAGCCGGTACCCAAAGTGGTCTAACGCCTATGACTTCAACTTTTCGGAAACCGAGAAGTACAAGGATATAGCCGGATTTTATAGAGAGG |
| | | TGGAAGAGCAGGGCTACAAGGTGTCATTCGAGTCCGCCAGCAAGAAGGAAGTGGACAAGCTCGTGGAAGAGGGTAAGCTCTACATGT |
| | | TCCAGATTTATAATAAAGACTTTAGCGATAAGAGCCACGGGACACCTAATCTCCACACAATGTATTTCAAGCTGCTCTTCGACGAGAATA |
| | | ACCACGGCCAAATCAGGTTGTCAGGAGGGGCTGAACTCTTCATGCGGCGCGCTAGCCTTAAGAAGGAGGAGCTTGTAGTCCACCCTG |
| | | CGAATAGTCCAATTGCGAATAAGAACCCGGACAATCCTAAAAAGACTACAACATTGAGCTACGACGTGTACAAGGATAAGAGGTTTTCC |
| | | GAGGATCAGTACGAGCTCCACATCCCGATTGCGATCAACAAGTGCCCAAAGAATATTTTCAAGATAAACACAGAGGTGCGTGTACTCCT |
| | | GAAGCATGACGACAATCCTTACGTCATTGGGATTGATCGGGGCGAGAGGAACCTCCTCTATATTGTGGTGGTGGACGGGAAGGGGAA |
| | | CATAGTCGAACAGTACTCCCTTAACGAAATAATTAACAATTTCAACGGCATCCGTATCAAGACCGACTACCATTCGTTGCTGGACAAGAA |
| | | GGAGAAGGAGAGATTTGAGGCGCGGCAAAATTGGACAAGTATCGAGAACATCAAGGAACTCAAAGCAGGTTATATCTCTCAAGTTGTG |
| | | CATAAGATATGCGAGCTGGTTGAGAAGTATGACGCAGTGATCGCTCTTGAGGACCTCAACTCGGGCTTTAAGAATTCTAGAGTTAAAGT |
| | | GGAGAAGCAGGTCTATCAAAAGTTCGAGAAGATGCTTATAGATAAGCTCAACTACATGGTCGATAAGAAATCGAACCCATGTGCCACCG |
| | | GCGGCGCACTCAAAGGTTACCAAATAACAAACAAATTCGAGTCCTTCAAATCGATGAGTACTCAGAATGGGTTCATATTTTATATACCGG |
| | | CGTGGCTTACGTCTAAGATCGACCCGTCAACTGGTTTTGTCAACCTGTTGAAGACGAAATACACGTCCATTGCCGATTCAAAAAAGTTC |
| | | ATATCTAGTTTTGATCGTATTATGTACGTCCCAGAGGAAGATCTTTTCGAGTTTGCTCTCGACTACAAAAACTTTTCGCGCACCGATGCG |
| | | GATTACATTAAAAAATGGAAACTCTATTCGTACGGCAACAGAATCAGGATTTTTCGCAACCCTAAGAAGAATAACGTCTTTGATTGGGAG |
| | | GAAGTTTGCTTGACTAGCGCGTACAAGGAGCTCTTTAATAAGTATGGCATTAACTACCAACAGGGTGATATCAGAGCACTGCTTTGCGA |
| | | ACAATCTGACAAGGCTTTCTACTCATCCTTCATGGCTTTGATGAGCCTGATGCTCCAGATGAGAAATTCAATTACAGGCAGAACCGACG |
| | | TGGATTTCTTGATCTCCCCGGTTAAAAATTCTGATGGCATCTTTTACGATAGCAGGAACTATGAAGCGCAAGAGAATGCGATTCTGCCAA |
| | | AAAATGCAGACGCCAACGGTGCCTATAACATCGCCAGGAAAGTCCTGTGGGCGATCGGCCAGTTCAAAAAGGCCGAAGACGAAAAATT |
| | | GGACAAGGTCAAAATCGCTATCAGCAACAAAGAGTGGCTGGAGTATGCTCAGACATCCGTAAAGCATAAGCGTCCTGCTGCCACCAAA |
| | | AAGGCCGGACAGGCTAAGAAAAAGAAGTGATTAATTAAGATCGTTCAAACATTTGGCAATAAAGTTTCTTAAGATTGAATCCTGTTGCCG |
| | | GTCTTGCGATGATTATCATATAATTTCTGTTGAATTACGTTAAGCATGTAATAATTAACATGTAATGCATGACGTTATTTATGAGATGGGTT |
| | | TTTATGATTAGAGTCCCGCAATTATACATTTAATACGCGATAGAAAACAAAATATAGCGCGCAAACTAGGATAAATTATCGCGCGCGGTG |
| | | TCATCTATGTTACTAGATCTTCGAAGAATTCATTATGTGGTCTAGGTAGGTTCTATATATAAGAAAACTTGAAATGTTCTAAAAAAAAATTC |
| | | AAGCCCATGCATGATTGAAGCAAACGGTATAGCAACGGTGTTAACCTGATCTAGTGATCTCTTGCAATCCTTAACGGCCACCTACCGCA |
| | | GGTAGCAAACGGCGTCCCCCTCCTCGATATCTCCGCGGCGACCTCTGGCTTTTTCCGCGGAATTGCGCGGTGGGGACGGATTCCACG |
| | | AGACCGCGACGCAACCGCCTCTCGCCGCTGGGCCCCACACCGCTCGGTGCCGTAGCCTCACGGGACTCTTTCTCCCTCCTCCCCCGT |
| | | TATAAATTGGCTTCATCCCCTCCTTGCCTCATCCATCCAAATCCCAGTCCCCAATCCCATCCCTTCGTAGGAGAAATTCATCGAAGCTAA |
| | | GCGAATCCTCGCGATCCTCTCAAGGTACTGCGAGTTTTCGATCCCCCTCTCGACCCCTCGTATGTTTGTGTTTGTCGTAGCGTTTGATT |
| | | AGGTATGCTTTCCCTGTTTGTGTTCGTCGTAGCGTTTGATTAGGTATGCTTTCCCTGTTCGTGTTCATCGTAGTGTTTGATTAGGTCGTG |
| | | TGAGGCGATGGCCTGCTCGCGTCCTTCGATCTGTAGTCGATTTGCGGGTCGTGGTGTAGATCTGCGGGCTGTGATGAAGTTATTTGGT |
| | | GTGATCTGCTCGCCTGATTCTGCGGGTTGGCTCGAGTAGATATGATGGTTGGACCGGTTGGTTCGTTTACCGCGCTAGGGTTGGGCTG |
| | | GGATGATGTTGCATGCGCCGTTGCGCGTGATCCCGCAGCAGGACTTGCGTTTGATTGCCAGATCTCGTTACGATTATGTGATTTGGTTT |
| | | GGACTTTTTAGATCTGTAGCTTCTGCTTATGTGCCAGATGCGCCTACTGCTCATATGCCTGATGATAATCATAAATGGCTGTGGAACTAA |
| | | CTAGTTGATTGCGGAGTCATGTATCAGCTACAGGTGTAGGGACTAGCTACAGGTGTAGGGACTTGCGTCTAATTGTTTGGTCCTTTACT |
| | | CATGTTGCAATTATGCAATTTAGTTTAGATTGTTTGTTCCACTCATCTAGGCTGTAAAAGGGACACTGCTTAGATTGCTGTTTAATCTTTTT |
| | | AGTAGATTATATTATATTGGTAACTTATTACCCCTATTACATGCCATACGTGACTTCTGCTCATGCCTGATGATAATCATAGATCACTGTG |
| | | GAATTAATTAGTTGATTGTTGAATCATGTTTCATGTACATACCACGGCACAATTGCTTAGTTCCTTAACAAATGCAAATTTTACTGATCCAT |
| | | GTATGATTTGCGTGGTTCTCTAATGTGAAATACTATAGCTACTTGTTAGTAAGAATCAGGTTCGTATGCTTAATGCTGTATGTGCCTTCTG |
| | | CTCATGCCTGATGATAATCATATATCACTGGAATTAATTAGTTGATCGTTTAATCATATATCAAGTACATACCATGCCACAATTTTTAGTCA |
| | | CTTAACCCATGCAGATTGAACTGGTCCCTGCATGTTTTGCTAAATTGTTCTATTCTGATTAGACCATATATCATGTATTTTTTTTTGGTAAT |
| | | GGTTCTCTTATTTTAAATGCTATATAGTTCTGGTACTTGTTAGAAAGATCTGCTTCATAGTTTAGTTGCCTATCCCTCGAATTAGGATGCT |
| | | GAGCAGCTGATCCTATAGCTTTGTTTCATGTATCAATTCTTTTGTGTTCAACAGTCAGTTTTTGTTAGATTCATTGTAACTTATGGTCGCTT |
| | | ACTCTTCTGGTCCTCAATGCTTGCAGGGGATCCAAATTACTGATGAGTCCGTGAGGACGAAACGAGTAAGCTCGTCTAATTTCTACTAA |
| | | GTGTAGATATGCATGTGCTAGCTGGTGGACTGGCCGGCATGGTCCCAGCCTCCTCGCTGGCGCCGGCTGGGCAACATGCTTCGGCAT |
| | | GGCGAATGGGACGATCGTTCAAACATTTGGCAATAAAGTTTCTTAAGATTGAATCCTGTTGCCGGTCTTGCGATGATTATCATATAATTT |
| | | CTGTTGAATTACGTTAAGCATGTAATAATTAACATGTAATGCATGACGTTATTTATGAGATGGGTTTTTATGATTAGAGTCCCGCAATTAT |
| | | ACATTTAATACGCGATAGAAAACAAAATATAGCGCGCAAACTAGGATAAATTATCGCGCGCGGTGTCATCTATGTTACTAGATCCGGAC |
| | | CGCGCCTGCAGTGCAGCGTGACCCGGTCGTGCCCCTCTCTAGAGATAATGAGCATTGCATGTCTAAGTTATAAAAAATTACCACATATT |
| | | TTTTTTGTCACACTTGTTTGAAGTGCAGTTTATCTATCTTTATACATATATTTAAACTTTACTCTACGAATAATATAATCTATAGTACTACAA |
| | | TAATATCAGTGTTTTAGAGAATCATATAAATGAACAGTTAGACATGGTCTAAAGGACAATTGAGTATTTTGACAACAGGACTCTACAGTTT |
| | | TATCTTTTTAGTGTGCATGTGTTCTCCTTTTTTTTTGCAAATAGCTTCACCTATATAATACTTCATCCATTTTATTAGTACATCCATTTAGGG |
| | | TTTAGGGTTAATGGTTTTTATAGACTAATTTTTTTAGTACATCTATTTTATTCTATTTTAGCCTCTAAATTAAGAAAACTAAAACTCTATTTTA |
| | | GTTTTTTTATTTAATAATTTAGATATAAAATAGAATAAAATAAAGTGACTAAAAATTAAACAAATACCCTTTAAGAAATTAAAAAAACTAAGG |
| | | AAACATTTTTCTTGTTTCGAGTAGATAATGCCAGCCTGTTAAACGCCGTCGACGAGTCTAACGGACACCAACCAGCGAACCAGCAGCGT |
| | | CGCGTCGGGCCAAGCGAAGCAGACGGCACGGCATCTCTGTCGCTGCCTCTGGACCCCTCTCGAGAGTTCCGCTCCACCGTTGGACTT |
| | | GCTCCGCTGTCGGCATCCAGAAATTGCGTGGCGGAGCGGCAGACGTGAGCCGGCACGGCAGGCGGCCTCCTCCTCCTCTCACGGCA |
| | | CCGGCAGCTACGGGGGATTCCTTTCCCACCGCTCCTTCGCTTTCCCTTCCTCGCCCGCCGTAATAAATAGACACCCCCTCCACACCCT |
| | | CTTTCCCCAACCTCGTGTTGTTCGGAGCGCACACACACACAACCAGATCTCCCCCAAATCCACCCGTCGGCACCTCCGCTTCAAGGTA |
| | | CGCCGCTCGTCCTCCCCCCCCCCCCTCTCTACCTTCTCTAGATCGGCGTTCCGGTCCATGGTTAGGGCCCGGTAGTTCTACTTCTGTT |
| | | CATGTTTGTGTTAGATCCGTGTTTGTGTTAGATCCGTGCTGCTAGCGTTCGTACACGGATGCGACCTGTACGTCAGACACGTTCTGATT |
| | | GCTAACTTGCCAGTGTTTCTCTTTGGGGAATCCTGGGATGGCTCTAGCCGTTCCGCAGACGGGATCGATTTCATGATTTTTTTTGTTTCG |
| | | TTGCATAGGGTTTGGTTTGCCCTTTTCCTTTATTTCAATATATGCCGTGCACTTGTTTGTCGGGTCATCTTTTCATGCTTTTTTTTGTCTTG |
| | | GTTGTGATGATGTGGTCTGGTTGGGGGGTCGTTCTAGATCGGAGTAGAATTCTGTTTCAAACTACCTGGTGGATTTATTAATTTTGGATC |
| | | TGTATGTGTGTGCCATACATATTCATAGTTACGAATTGAAGATGATGGATGGAAATATCGATCTAGGATAGGTATACATGTTGATGCGGG |
| | | TTTTACTGATGCATATACAGAGATGCTTTTTGTTCGCTTGGTTGTGATGATGTGGTGTGGTTGGGCGGTCGTTCATTCGTTCTAGATCGG |
| | | AGTAGAATACTGTTTCAAACTACCTGGTGTATTTATTAATTTTGGAACTGTATGTGTGTGTCATACATCTTCATAGTTACGAGTTTAAGAT |
| | | GGATGGAAATATCGATCTAGGATAGGTATACATGTTGATGTGGGTTTTACTGATGCATATACATGATGGCATATGCAGCATCTATTCATA |
| | | TGCTCTAACCTTGAGTACCTATCTATTATAATAAACAAGTATGTTTTATAATTATTTTGATCTTGATATACTTGGATGATGGCATATGCAGC |
| | | AGCTATATGTGGATTTTTTTAGCCCTGCCTTCATACGCTATTTATTTGCTTGGTACTGTTTCTTTTGTCGATGCTCACCCTGTTGTTTGGT |
| | | GTTACTTCTGCAGGGATCCGGCAGCAGCCATGCAGAAGCTGATCAACAGCGTGCAGAACTACGCCTGGGGCAGCAAGACCGCCCTGA |
| | | CCGAGCTGTACGGCATGGAGAACCCCAGCAGCCAGCCCATGGCCGAGCTGTGGATGGGCGCCCACCCCAAGAGCAGCAGCCGCGTG |
| | | CAGAACGCCGCCGGCGACATCGTGAGCCTGCGCGACGTGATCGAGAGCGACAAGAGCACCCTGCTGGGCGAGGCCGTGGCCAAGC |
| | | GCTTCGGCGAGCTGCCCTTCCTGTTCAAGGTGCTGTGCGCCGCCCAGCCCCTGAGCATCCAGGTGCACCCCAACAAGCACAACAGCG |
| | | AGATCGGCTTCGCCAAGGAGAACGCCGCCGGCATCCCCATGGACGCCGCCGAGCGCAACTACAAGGACCCCAACCACAAGCCCGAG |
| | | CTGGTGTTCGCCCTGACCCCCTTCCTGGCCATGAACGCCTTCCGCGAGTTCAGCGAGATCGTGAGCCTGCTGCAGCCCGTGGCCGGC |
| | | GCCCACCCCGCCATCGCCCACTTCCTGCAGCAGCCCGACGCCGAGCGCCTGAGCGAGCTGTTCGCCAGCCTGCTGAACATGCAGGG |
| | | CGAGGAGAAGAGCCGCGCCCTGGCCATCCTGAAGAGCGCCCTGGACAGCCAGCAGGGCGAGCCCTGGCAGACCATCCGCCTGATCA |
| | | GCGAGTTCTACCCCGAGGACAGCGGCCTGTTCAGCCCCCTGCTGCTGAACGTGGTGAAGCTGAACCCCGGCGAGGCCATGTTCCTGT |
| | | TCGCCGAGACCCCCCACGCCTACCTGCAGGGCGTGGCCCTGGAGGTGATGGCCAACAGCGACAACGTGCTGCGCGCCGGCCTGACC |
| | | CCCAAGTACATCGACATCCCCGAGCTGGTGGCCAACGTGAAGTTCGAGGCCAAGCCCGCCAACCAGCTGCTGACCCAGCCCGTGAAG |
| | | CAGGGCGCCGAGCTGGACTTCCCCATCCCCGTGGACGACTTCGCCTTCAGCCTGCACGACCTGAGCGACAAGGAGACCACCATCAGC |
| | | CAGCAGAGCGCCGCCATCCTGTTCTGCGTGGAGGGCGACGCCACCCTGTGGAAGGGCAGCCAGCAGCTGCAGCTGAAGCCCGGCGA |
| | | GAGCGCCTTCATCGCCGCCAACGAGAGCCCCGTGACCGTGAAGGGCCACGGCCGCCTGGCCCGCGTGTACAACAAGCTGTGATAGG |
| | | AGCTCGATCCGTCGACCTGCAGATCGTTCAAACATTTGGCAATAAAGTTTCTTAAGATTGAATCCTGTTGCCGGTCTTGCGATGATTATC |
| | | ATATAATTTCTGTTGAATTACGTTAAGCATGTAATAATTAACATGTAATGCATGACGTTATTTATGAGATGGGTTTTTATGATTAGAGTCCC |
| | | GCAATTATACATTTAATACGCGATAGAAAACAAAATATAGCGCGCAAACTAGGATAAATTATCGCGCGCGGTGTCATCTATGTTACTAGA |
| | | TCGGCGCGCCGCAATTGAAGTTTGGGGGGCCAGCATGGCCGTATCCGCAATGTGTTATTAAGTTGTCTAAGCGTCAATTTGTTTACACC |
| | | ACAATATATCCTGCCACCAGCCAGCCAACAGCTCCCCGACCGGCAGCTCGGCACAAAATCACCACTCGATACAGGCAGCCCATCAGAA |
| | | TTAATTCTCATGTTTGACAGCTTATCATCGACTGCACGGTGCACCAATGCTTCTGGCGTCAGGCAGCCATCGGAAGCTGTGGTATGGCT |
| | | GTGCAGGTCGTAAATCACTGCATAATTCGTGTCGCTCAAGGCGCACTCCCGTTCTGGATAATGTTTTTTGCGCCGACATCATAACGGTT |
| | | CTGGCAAATATTCTGAAATGAGCTGTTGACAATTAATCATCCGGCTCGTATAATGTGTGGAATTGTGAGCGGATAACAATTTCACACAGG |
| | | AAACAGACCATGAGGGAAGCGTTGATCGCCGAAGTATCGACTCAACTATCAGAGGTAGTTGGCGTCATCGAGCGCCATCTCGAACCGA |
| | | CGTTGCTGGCCGTACATTTGTACGGCTCCGCAGTGGATGGCGGCCTGAAGCCACACAGTGATATTGATTTGCTGGTTACGGTGACCGT |
| | | AAGGCTTGATGAAACAACGCGGCGAGCTTTGATCAACGACCTTTTGGAAACTTCGGCTTCCCCTGGAGAGAGCGAGATTCTCCGCGCT |
| | | GTAGAAGTCACCATTGTTGTGCACGACGACATCATTCCGTGGCGTTATCCAGCTAAGCGCGAACTGCAATTTGGAGAATGGCAGCGCA |
| | | ATGACATTCTTGCAGGTATCTTCGAGCCAGCCACGATCGACATTGATCTGGCTATCTTGCTGACAAAAGCAAGAGAACATAGCGTTGCC |
| | | TTGGTAGGTCCAGCGGCGGAGGAACTCTTTGATCCGGTTCCTGAACAGGATCTATTTGAGGCGCTAAATGAAACCTTAACGCTATGGAA |
| | | CTCGCCGCCCGACTGGGCTGGCGATGAGCGAAATGTAGTGCTTACGTTGTCCCGCATTTGGTACAGCGCAGTAACCGGCAAAATCGC |
| | | GCCGAAGGATGTCGCTGCCGACTGGGCAATGGAGCGCCTGCCGGCCCAGTATCAGCCCGTCATACTTGAAGCTAGGCAGGCTTATCT |
| | | TGGACAAGAAGATCGCTTGGCCTCGCGCGCAGATCAGTTGGAAGAATTTGTTCACTACGTGAAAGGCGAGATCACCAAAGTAGTCGGC |
| | | AAATAAAGCTCTAGTGGATCTCCGTACCCGGGGATCTGGCTCGCGGCGGACGCACGACGCCGGGGCGAGACCATAGGCGATCTCCTA |
| | | AATCAATAGTAGCTGTAACCTCGAAGCGTTTCACTTGTAACAACGATTGAGAATTTTTGTCATAAAATTGAAATACTTGGTTCGCATTTTT |
| | | GTCATCCGCGGTCAGCCGCAATTCTGACGAACTGCCCATTTAGCTGGAGATGATTGTACATCCTTCACGTGAAAATTTCTCAAGCGCTG |
| | | TGAACAAGGGTTCAGATTTTAGATTGAAAGGTGAGCCGTTGAAACACGTTCTTCTTGTCGATGACGACGTCGCTATGCGGCATCTTATT |
| | | ATTGAATACCTTACGATCCACGCCTTCAAAGTGACCGCGGTAGCCGACAGCACCCAGTTCACAAGAGTACTCTCTTCCGCGACGGTCG |
| | | ATGTCGTGGTTGTTGATCTAGATTTAGGTCGTGAAGATGGGCTCGAGCTAGGAGCAAGTGATTTTATCGCTAAGCCGTTCAGTATCAGA |
| | | GAGTTTCTAGCACGCATTCGGGTTGCCTTGCGCGTGCGCCCCAACGTTGTCCGCTCCAAAGACCGACGGTCTTTTTGTTTTACTGACTG |
| | | GACACTTAATCTCAGGCAACGTCGCTTGATGTCCGAAGCTGGCGGTGAGGTGAAACTTACGGCAGGTGAGTTCAATCTTCTCCTCGCG |
| | | TTTTTAGAGAAACCCCGCGACGTTCTATCGCGCGAGCAACTTCTCATTGCCAGTCGAGTACGCGACGAGGAGGTTTATGACAGGAGTA |
| | | TAGATGTTCTCATTTTGAGGCTGCGCCGCAAACTTGAGGCAGATCCGTCAAGCCCTCAACTGATAAAAACAGCAAGAGGTGCCGGTTAT |
| | | TTCTTTGACGCGGACGTGCAGGTTTCGCACGGGGGGACGATGGCAGCCTGAGCCAATTCCCAGATCCCCGAGGAATCGGCGTGAGC |
| | | GGTCGCAAACCATCCGGCCCGGTACAAATCGGCGCGGCGCTGGGTGATGACCTGGTGGAGAAGTTGAAGGCCGCGCAGGCCGCCCA |
| | | GCGGCAACGCATCGAGGCAGAAGCACGCCCCGGTGAATCGTGGCAAGCGGCCGCTGATCGAATCCGCAAAGAATCCCGGCAACCGC |
| | | CGGCAGCCGGTGCGCCGTCGATTAGGAAGCCGCCCAAGGGCGACGAGCAACCAGATTTTTTCGTTCCGATGCTCTATGACGTGGGCA |
| | | CCCGCGATAGTCGCAGCATCATGGACGTGGCCGTTTTCCGTCTGTCGAAGCGTGACCGACGAGCTGGCGAGGTGATCCGCTACGAGC |
| | | TTCCAGACGGGCACGTAGAGGTTTCCGCAGGGCCGGCCGGCATGGCCAGTGTGTGGGATTACGACCTGGTACTGATGGCGGTTTCCC |
| | | ATCTAACCGAATCCATGAACCGATACCGGGAAGGGAAGGGAGACAAGCCCGGCCGCGTGTTCCGTCCACACGTTGCGGACGTACTCA |
| | | AGTTCTGCCGGCGAGCCGATGGCGGAAAGCAGAAAGACGACCTGGTAGAAACCTGCATTCGGTTAAACACCACGCACGTTGCCATGC |
| | | AGCGTACGAAGAAGGCCAAGAACGGCCGCCTGGTGACGGTATCCGAGGGTGAAGCCTTGATTAGCCGCTACAAGATCGTAAAGAGCG |
| | | AAACCGGGCGGCCGGAGTACATCGAGATCGAGCTAGCTGATTGGATGTACCGCGAGATCACAGAAGGCAAGAACCCGGACGTGCTGA |
| | | CGGTTCACCCCGATTACTTTTTGATCGATCCCGGCATCGGCCGTTTTCTCTACCGCCTGGCACGCCGCGCCGCAGGCAAGGCAGAAG |
| | | CCAGATGGTTGTTCAAGACGATCTACGAACGCAGTGGCAGCGCCGGAGAGTTCAAGAAGTTCTGTTTCACCGTGCGCAAGCTGATCGG |
| | | GTCAAATGACCTGCCGGAGTACGATTTGAAGGAGGAGGCGGGGCAGGCTGGCCCGATCCTAGTCATGCGCTACCGCAACCTGATCGA |
| | | GGGCGAAGCATCCGCCGGTTCCTAATGTACGGAGCAGATGCTAGGGCAAATTGCCCTAGCAGGGGAAAAAGGTCGAAAAGGTCTCTT |
| | | TCCTGTGGATAGCACGTACATTGGGAACCCAAAGCCGTACATTGGGAACCGGAACCCGTACATTGGGAACCCAAAGCCGTACATTGGG |
| | | AACCGGTCACACATGTAAGTGACTGATATAAAAGAGAAAAAAGGCGATTTTTCCGCCTAAAACTCTTTAAAACTTATTAAAACTCTTAAAA |
| | | CCCGCCTGGCCTGTGCATAACTGTCTGGCCAGCGCACAGCCGAAGAGCTGCAAAAAGCGCCTACCCTTCGGTCGCTGCGCTCCCTAC |
| | | GCCCCGCCGCTTCGCGTCGGCCTATCGCGGCCGCTGGCCGCTCAAAAATGGCTGGCCTACGGCCAGGCAATCTACCAGGGCGCGGA |
| | | CAAGCCGCGCCGTCGCCACTCGACCGCCGGCGCTGAGGTCTGCCTCGTGAAGAAGGTGTTGCTGACTCATACCAGGCCTGAATCGCC |
| | | CCATCATCCAGCCAGAAAGTGAGGGAGCCACGGTTGATGAGAGCTTTGTTGTAGGTGGACCAGTTGGTGATTTTGAACTTTTGCTTTGC |
| | | CACGGAACGGTCTGCGTTGTCGGGAAGATGCGTGATCTGATCCTTCAACTCAGCAAAAGTTCGATTTATTCAACAAAGCCGCCGTCCC |
| | | GTCAAGTCAGCGTAATGCTCTGCCAGTGTTACAACCAATTAACCAATTCTGATTAGAAAAACTCATCGAGCATCAAATGAAACTGCAATT |
| | | TATTCATATCAGGATTATCAATACCATATTTTTGAAAAAGCCGTTTCTGTAATGAAGGAGAAAACTCACCGAGGCAGTTCCATAGGATGG |
| | | CAAGATCCTGGTATCGGTCTGCGATTCCGACTCGTCCAACATCAATACAACCTATTAATTTCCCCTCGTCAAAAATAAGGTTATCAAGTG |
| | | AGAAATCACCATGAGTGACGACTGAATCCGGTGAGAATGGCAAAAGCTCTGCATTAATGAATCGGCCAACGCGCGGGGAGAGGCGGT |
| | | TTGCGTATTGGGCGCTCTTCCGCTTCCTCGCTCACTGACTCGCTGCGCTCGGTCGTTCGGCTGCGGCGAGCGGTATCAGCTCACTCAA |
| | | AGGCGGTAATACGGTTATCCACAGAATCAGGGGATAACGCAGGAAAGAACATGTGAGCAAAAGGCCAGCAAAAGGCCAGGAACCGTA |
| | | AAAAGGCCGCGTTGCTGGCGTTTTTCCATAGGCTCCGCCCCCCTGACGAGCATCACAAAAATCGACGCTCAAGTCAGAGGTGGCGAAA |
| | | CCCGACAGGACTATAAAGATACCAGGCGTTTCCCCCTGGAAGCTCCCTCGTGCGCTCTCCTGTTCCGACCCTGCCGCTTACCGGATAC |
| | | CTGTCCGCCTTTCTCCCTTCGGGAAGCGTGGCGCTTTCTCATAGCTCACGCTGTAGGTATCTCAGTTCGGTGTAGGTCGTTCGCTCCAA |
| | | GCTGGGCTGTGTGCACGAACCCCCCGTTCAGCCCGACCGCTGCGCCTTATCCGGTAACTATCGTCTTGAGTCCAACCCGGTAAGACA |
| | | CGACTTATCGCCACTGGCAGCAGCCACTGGTAACAGGATTAGCAGAGCGAGGTATGTAGGCGGTGCTACAGAGTTCTTGAAGTGGTG |
| | | GCCTAACTACGGCTACACTAGAAGAACAGTATTTGGTATCTGCGCTCTGCTGAAGCCAGTTACCTTCGGAAAAAGAGTTGGTAGCTCTT |
| | | GATCCGGCAAACAAACCACCGCTGGTAGCGGTGGTTTTTTTGTTTGCAAGCAGCAGATTACGCGCAGAAAAAAAGGATCTCAAGAAGAT |
| | | CCTTTGATCTTTTCTACGGGGTCTGACGCTCAGTGGAACGAAAACTCACGTTAAGGGATTTTGGTCATGAGATTATCAAAAAGGATCTTC |
| | | ACCTAGATCCTTTTGATCCGGACAAACAAACAAATACAGTAATTTAGCCAGGACGTCGGCCGAAAGAGCGACAAGCAGATCACGCTTTT |
| | | CGACAGCGTCGGATTTGCGATCGAGGATTTTTCGGCGCTGCGCTACGTCCGCGACCGCGTTGAGGGATCAAGCCACAGCAGCCCACT |
| | | CGACCTTCTAGCCGACCCAGACGAGCCAAGGGATCTTTTTGGAATGCTGCTCCGTCGTCAGGCTTTCCGACGTTTGGGTGGTTGAACA |
| | | GAAGTCATTATCGCACGGAATGCCAAGCACTCCCGAGGGG |
| 313 | CPF1 RICE | AACCCTGTGGTTGGCATGCACATACAAATGGACGAACGGATAAACCTTTTCACGCCCTTTTAAATATCCGATTATTCTAATAAACGCTCT |
| | EXPRESSION VECTOR | TTTCTCTTAGGTTTACCCGCCAATATATCCTGTCAAACACTGATAGTTTAAACTGGCACTAGCCTAACGGTGTTGACTAACTAGGCCGCT |
| | (SEQ ID NO: 313) | TCCCTAATTAGCTAAGGTACCGGCGCGCCACCGGTGAGCTCGGGACCCGAATTCATTATGTGGTCTAGGTAGGTTCTATATATAAGAAA |
| | | ACTTGAAATGTTCTAAAAAAAAATTCAAGCCCATGCATGATTGAAGCAAACGGTATAGCAACGGTGTTAACCTGATCTAGTGATCTCTTG |
| | | CAATCCTTAACGGCCACCTACCGCAGGTAGCAAACGGCGTCCCCCTCCTCGATATCTCCGCGGCGACCTCTGGCTTTTTCCGCGGAAT |
| | | TGCGCGGTGGGGACGGATTCCACGAGACCGCGACGCAACCGCCTCTCGCCGCTGGGCCCCACACCGCTCGGTGCCGTAGCCTCACG |
| | | GGACTCTTTCTCCCTCCTCCCCCGTTATAAATTGGCTTCATCCCCTCCTTGCCTCATCCATCCAAATCCCAGTCCCCAATCCCATCCCTT |
| | | CGTAGGAGAAATTCATCGAAGCTAAGCGAATCCTCGCGATCCTCTCAAGGTACTGCGAGTTTTCGATCCCCCTCTCGACCCCTCGTATG |
| | | TTTGTGTTTGTCGTAGCGTTTGATTAGGTATGCTTTCCCTGTTTGTGTTCGTCGTAGCGTTTGATTAGGTATGCTTTCCCTGTTCGTGTTC |
| | | ATCGTAGTGTTTGATTAGGTCGTGTGAGGCGATGGCCTGCTCGCGTCCTTCGATCTGTAGTCGATTTGCGGGTCGTGGTGTAGATCTG |
| | | CGGGCTGTGATGAAGTTATTTGGTGTGATCTGCTCGCCTGATTCTGCGGGTTGGCTCGAGTAGATATGATGGTTGGACCGGTTGGTTC |
| | | GTTTACCGCGCTAGGGTTGGGCTGGGATGATGTTGCATGCGCCGTTGCGCGTGATCCCGCAGCAGGACTTGCGTTTGATTGCCAGAT |
| | | CTCGTTACGATTATGTGATTTGGTTTGGACTTTTTAGATCTGTAGCTTCTGCTTATGTGCCAGATGCGCCTACTGCTCATATGCCTGATG |
| | | ATAATCATAAATGGCTGTGGAACTAACTAGTTGATTGCGGAGTCATGTATCAGCTACAGGTGTAGGGACTAGCTACAGGTGTAGGGACT |
| | | TGCGTCTAATTGTTTGGTCCTTTACTCATGTTGCAATTATGCAATTTAGTTTAGATTGTTTGTTCCACTCATCTAGGCTGTAAAAGGGACA |
| | | CTGCTTAGATTGCTGTTTAATCTTTTTAGTAGATTATATTATATTGGTAACTTATTACCCCTATTACATGCCATACGTGACTTCTGCTCATG |
| | | CCTGATGATAATCATAGATCACTGTGGAATTAATTAGTTGATTGTTGAATCATGTTTCATGTACATACCACGGCACAATTGCTTAGTTCCT |
| | | TAACAAATGCAAATTTTACTGATCCATGTATGATTTGCGTGGTTCTCTAATGTGAAATACTATAGCTACTTGTTAGTAAGAATCAGGTTCG |
| | | TATGCTTAATGCTGTATGTGCCTTCTGCTCATGCCTGATGATAATCATATATCACTGGAATTAATTAGTTGATCGTTTAATCATATATCAAG |
| | | TACATACCATGCCACAATTTTTAGTCACTTAACCCATGCAGATTGAACTGGTCCCTGCATGTTTTGCTAAATTGTTCTATTCTGATTAGAC |
| | | CATATATCATGTATTTTTTTTTGGTAATGGTTCTCTTATTTTAAATGCTATATAGTTCTGGTACTTGTTAGAAAGATCTGCTTCATAGTTTAG |
| | | TTGCCTATCCCTCGAATTAGGATGCTGAGCAGCTGATCCTATAGCTTTGTTTCATGTATCAATTCTTTTGTGTTCAACAGTCAGTTTTTGT |
| | | TAGATTCATTGTAACTTATGGTCGCTTACTCTTCTGGTCCTCAATGCTTGCAGGGATCCCCTAAATAGACCATGGCTCCTAAGAAGAAGC |
| | | GGAAGGTTGGTATTCACGGGGTGCCTGCGGCTTCAAAGCTCGAGAAATTCACCAACTGTTATTCGTTGAGCAAAACACTGCGGTTTAAA |
| | | GCGATTCCAGTCGGCAAGACTCAAGAGAATATAGACAATAAGCGGCTGTTGGTGGAAGATGAAAAGCGCGCGGAAGACTACAAAGGG |
| | | GTGAAGAAGTTGTTGGACAGATACTACCTCTCTTTTATCAATGATGTCTTGCACTCAATCAAATTGAAGAATCTGAACAACTACATCTCCC |
| | | TCTTCAGAAAGAAAACAAGGACAGAAAAGGAGAATAAGGAACTTGAAAATTTGGAGATCAATCTGAGGAAAGAGATCGCGAAAGCCTTT |
| | | AAAGGCAACGAAGGATACAAAAGTCTGTTCAAGAAGGATATAATTGAGACAATTTTGCCAGAGTTCCTCGATGACAAGGACGAGATTGC |
| | | GCTGGTCAATTCGTTCAACGGATTCACAACAGCATTCACAGGCTTCTTTGATAATCGGGAAAATATGTTCTCTGAGGAGGCAAAGTCCA |
| | | CTTCTATTGCGTTCAGGTGTATCAATGAGAATCTCACTAGGTACATTTCCAACATGGATATCTTTGAGAAGGTTGACGCAATTTTTGACAA |
| | | GCACGAAGTTCAGGAGATTAAGGAGAAGATCCTCAATTCCGATTATGACGTTGAGGACTTCTTCGAGGGTGAGTTTTTTAATTTCGTGCT |
| | | CACTCAAGAGGGTATCGACGTGTATAATGCGATCATCGGTGGGTTCGTGACTGAGTCCGGTGAAAAGATTAAGGGATTGAACGAGTAT |
| | | ATCAACCTTTACAACCAAAAGACGAAACAGAAGCTGCCAAAGTTCAAGCCTCTTTACAAACAGGTTCTTTCAGACCGCGAGTCACTCTC |
| | | GTTCTATGGGGAGGGCTACACTTCGGATGAGGAAGTCCTGGAGGTGTTCAGGAATACTCTCAATAAGAATTCGGAGATTTTCTCTTCTA |
| | | TAAAAAAACTGGAAAAGTTGTTTAAGAATTTTGACGAATACTCTAGCGCCGGCATATTTGTGAAAAACGGCCCGGCCATATCAACGATAA |
| | | GTAAAGATATCTTCGGCGAATGGAACGTGATCAGAGACAAATGGAACGCGGAGTATGACGATATTCACCTGAAGAAGAAGGCTGTCGT |
| | | AACGGAGAAGTACGAGGATGATCGCAGGAAAAGCTTCAAAAAGATCGGAAGTTTCAGCCTGGAACAGTTGCAGGAGTATGCTGACGCC |
| | | GATCTTAGCGTCGTCGAGAAGTTGAAGGAGATAATCATCCAAAAGGTCGACGAGATATATAAAGTCTATGGATCAAGTGAAAAACTGTT |
| | | CGACGCCGACTTCGTTTTGGAGAAGTCCCTGAAGAAGAACGACGCTGTTGTTGCCATTATGAAGGATCTGCTCGACAGCGTGAAGAGT |
| | | TTCGAGAACTATATTAAGGCTTTTTTCGGGGAGGGGAAGGAGACTAACAGAGATGAGTCCTTCTACGGAGACTTCGTCCTCGCGTACG |
| | | ATATACTCCTTAAGGTAGACCACATCTACGACGCAATCAGAAATTACGTGACACAAAAGCCGTACAGCAAGGACAAGTTCAAACTCTACT |
| | | TCCAGAACCCCCAGTTCATGGGCGGCTGGGACAAGGACAAGGAAACGGATTACAGGGCTACGATCCTGAGGTATGGTTCAAAATACTA |
| | | CTTGGCGATTATGGACAAGAAGTACGCCAAGTGTCTCCAGAAGATTGACAAAGACGATGTCAATGGCAATTATGAGAAGATCAACTACA |
| | | AGCTGCTTCCGGGTCCGAACAAGATGCTCCCAAAGGTTTTCTTCAGCAAGAAATGGATGGCCTACTATAACCCAAGCGAGGACATCCA |
| | | GAAGATTTATAAGAACGGTACGTTCAAGAAGGGCGACATGTTCAATCTTAACGACTGTCACAAGCTGATCGACTTCTTCAAAGACTCAAT |
| | | TAGCCGGTACCCAAAGTGGTCTAACGCCTATGACTTCAACTTTTCGGAAACCGAGAAGTACAAGGATATAGCCGGATTTTATAGAGAGG |
| | | TGGAAGAGCAGGGCTACAAGGTGTCATTCGAGTCCGCCAGCAAGAAGGAAGTGGACAAGCTCGTGGAAGAGGGTAAGCTCTACATGT |
| | | TCCAGATTTATAATAAAGACTTTAGCGATAAGAGCCACGGGACACCTAATCTCCACACAATGTATTTCAAGCTGCTCTTCGACGAGAATA |
| | | ACCACGGCCAAATCAGGTTGTCAGGAGGGGCTGAACTCTTCATGCGGCGCGCTAGCCTTAAGAAGGAGGAGCTTGTAGTCCACCCTG |
| | | CGAATAGTCCAATTGCGAATAAGAACCCGGACAATCCTAAAAAGACTACAACATTGAGCTACGACGTGTACAAGGATAAGAGGTTTTCC |
| | | GAGGATCAGTACGAGCTCCACATCCCGATTGCGATCAACAAGTGCCCAAAGAATATTTTCAAGATAAACACAGAGGTGCGTGTACTCCT |
| | | GAAGCATGACGACAATCCTTACGTCATTGGGATTGATCGGGGCGAGAGGAACCTCCTCTATATTGTGGTGGTGGACGGGAAGGGGAA |
| | | CATAGTCGAACAGTACTCCCTTAACGAAATAATTAACAATTTCAACGGCATCCGTATCAAGACCGACTACCATTCGTTGCTGGACAAGAA |
| | | GGAGAAGGAGAGATTTGAGGCGCGGCAAAATTGGACAAGTATCGAGAACATCAAGGAACTCAAAGCAGGTTATATCTCTCAAGTTGTG |
| | | CATAAGATATGCGAGCTGGTTGAGAAGTATGACGCAGTGATCGCTCTTGAGGACCTCAACTCGGGCTTTAAGAATTCTAGAGTTAAAGT |
| | | GGAGAAGCAGGTCTATCAAAAGTTCGAGAAGATGCTTATAGATAAGCTCAACTACATGGTCGATAAGAAATCGAACCCATGTGCCACCG |
| | | GCGGCGCACTCAAAGGTTACCAAATAACAAACAAATTCGAGTCCTTCAAATCGATGAGTACTCAGAATGGGTTCATATTTTATATACCGG |
| | | CGTGGCTTACGTCTAAGATCGACCCGTCAACTGGTTTTGTCAACCTGTTGAAGACGAAATACACGTCCATTGCCGATTCAAAAAAGTTC |
| | | ATATCTAGTTTTGATCGTATTATGTACGTCCCAGAGGAAGATCTTTTCGAGTTTGCTCTCGACTACAAAAACTTTTCGCGCACCGATGCG |
| | | GATTACATTAAAAAATGGAAACTCTATTCGTACGGCAACAGAATCAGGATTTTTCGCAACCCTAAGAAGAATAACGTCTTTGATTGGGAG |
| | | GAAGTTTGCTTGACTAGCGCGTACAAGGAGCTCTTTAATAAGTATGGCATTAACTACCAACAGGGTGATATCAGAGCACTGCTTTGCGA |
| | | ACAATCTGACAAGGCTTTCTACTCATCCTTCATGGCTTTGATGAGCCTGATGCTCCAGATGAGAAATTCAATTACAGGCAGAACCGACG |
| | | TGGATTTCTTGATCTCCCCGGTTAAAAATTCTGATGGCATCTTTTACGATAGCAGGAACTATGAAGCGCAAGAGAATGCGATTCTGCCAA |
| | | AAAATGCAGACGCCAACGGTGCCTATAACATCGCCAGGAAAGTCCTGTGGGCGATCGGCCAGTTCAAAAAGGCCGAAGACGAAAAATT |
| | | GGACAAGGTCAAAATCGCTATCAGCAACAAAGAGTGGCTGGAGTATGCTCAGACATCCGTAAAGCATAAGCGTCCTGCTGCCACCAAA |
| | | AAGGCCGGACAGGCTAAGAAAAAGAAGTGATTAATTAAGATCGTTCAAACATTTGGCAATAAAGTTTCTTAAGATTGAATCCTGTTGCCG |
| | | GTCTTGCGATGATTATCATATAATTTCTGTTGAATTACGTTAAGCATGTAATAATTAACATGTAATGCATGACGTTATTTATGAGATGGGTT |
| | | TTTATGATTAGAGTCCCGCAATTATACATTTAATACGCGATAGAAAACAAAATATAGCGCGCAAACTAGGATAAATTATCGCGCGCGGTG |
| | | TCATCTATGTTACTAGATCTTCGAAGAATTCATTATGTGGTCTAGGTAGGTTCTATATATAAGAAAACTTGAAATGTTCTAAAAAAAAATTC |
| | | AAGCCCATGCATGATTGAAGCAAACGGTATAGCAACGGTGTTAACCTGATCTAGTGATCTCTTGCAATCCTTAACGGCCACCTACCGCA |
| | | GGTAGCAAACGGCGTCCCCCTCCTCGATATCTCCGCGGCGACCTCTGGCTTTTTCCGCGGAATTGCGCGGTGGGGACGGATTCCACG |
| | | AGACCGCGACGCAACCGCCTCTCGCCGCTGGGCCCCACACCGCTCGGTGCCGTAGCCTCACGGGACTCTTTCTCCCTCCTCCCCCGT |
| | | TATAAATTGGCTTCATCCCCTCCTTGCCTCATCCATCCAAATCCCAGTCCCCAATCCCATCCCTTCGTAGGAGAAATTCATCGAAGCTAA |
| | | GCGAATCCTCGCGATCCTCTCAAGGTACTGCGAGTTTTCGATCCCCCTCTCGACCCCTCGTATGTTTGTGTTTGTCGTAGCGTTTGATT |
| | | AGGTATGCTTTCCCTGTTTGTGTTCGTCGTAGCGTTTGATTAGGTATGCTTTCCCTGTTCGTGTTCATCGTAGTGTTTGATTAGGTCGTG |
| | | TGAGGCGATGGCCTGCTCGCGTCCTTCGATCTGTAGTCGATTTGCGGGTCGTGGTGTAGATCTGCGGGCTGTGATGAAGTTATTTGGT |
| | | GTGATCTGCTCGCCTGATTCTGCGGGTTGGCTCGAGTAGATATGATGGTTGGACCGGTTGGTTCGTTTACCGCGCTAGGGTTGGGCTG |
| | | GGATGATGTTGCATGCGCCGTTGCGCGTGATCCCGCAGCAGGACTTGCGTTTGATTGCCAGATCTCGTTACGATTATGTGATTTGGTTT |
| | | GGACTTTTTAGATCTGTAGCTTCTGCTTATGTGCCAGATGCGCCTACTGCTCATATGCCTGATGATAATCATAAATGGCTGTGGAACTAA |
| | | CTAGTTGATTGCGGAGTCATGTATCAGCTACAGGTGTAGGGACTAGCTACAGGTGTAGGGACTTGCGTCTAATTGTTTGGTCCTTTACT |
| | | CATGTTGCAATTATGCAATTTAGTTTAGATTGTTTGTTCCACTCATCTAGGCTGTAAAAGGGACACTGCTTAGATTGCTGTTTAATCTTTTT |
| | | AGTAGATTATATTATATTGGTAACTTATTACCCCTATTACATGCCATACGTGACTTCTGCTCATGCCTGATGATAATCATAGATCACTGTG |
| | | GAATTAATTAGTTGATTGTTGAATCATGTTTCATGTACATACCACGGCACAATTGCTTAGTTCCTTAACAAATGCAAATTTTACTGATCCAT |
| | | GTATGATTTGCGTGGTTCTCTAATGTGAAATACTATAGCTACTTGTTAGTAAGAATCAGGTTCGTATGCTTAATGCTGTATGTGCCTTCTG |
| | | CTCATGCCTGATGATAATCATATATCACTGGAATTAATTAGTTGATCGTTTAATCATATATCAAGTACATACCATGCCACAATTTTTAGTCA |
| | | CTTAACCCATGCAGATTGAACTGGTCCCTGCATGTTTTGCTAAATTGTTCTATTCTGATTAGACCATATATCATGTATTTTTTTTTGGTAAT |
| | | GGTTCTCTTATTTTAAATGCTATATAGTTCTGGTACTTGTTAGAAAGATCTGCTTCATAGTTTAGTTGCCTATCCCTCGAATTAGGATGCT |
| | | GAGCAGCTGATCCTATAGCTTTGTTTCATGTATCAATTCTTTTGTGTTCAACAGTCAGTTTTTGTTAGATTCATTGTAACTTATGGTCGCTT |
| | | ACTCTTCTGGTCCTCAATGCTTGCAGGGGATCCAAATTACTGATGAGTCCGTGAGGACGAAACGAGTAAGCTCGTCTAATTTCTACTAA |
| | | GTGTAGATATGCATGTGCTAGTTGGTGGACAGGCCGGCATGGTCCCAGCCTCCTCGCTGGCGCCGGCTGGGCAACATGCTTCGGCAT |
| | | GGCGAATGGGACGATCGTTCAAACATTTGGCAATAAAGTTTCTTAAGATTGAATCCTGTTGCCGGTCTTGCGATGATTATCATATAATTT |
| | | CTGTTGAATTACGTTAAGCATGTAATAATTAACATGTAATGCATGACGTTATTTATGAGATGGGTTTTTATGATTAGAGTCCCGCAATTAT |
| | | ACATTTAATACGCGATAGAAAACAAAATATAGCGCGCAAACTAGGATAAATTATCGCGCGCGGTGTCATCTATGTTACTAGATCCGGAC |
| | | CGCGCCTGCAGTGCAGCGTGACCCGGTCGTGCCCCTCTCTAGAGATAATGAGCATTGCATGTCTAAGTTATAAAAAATTACCACATATT |
| | | TTTTTTGTCACACTTGTTTGAAGTGCAGTTTATCTATCTTTATACATATATTTAAACTTTACTCTACGAATAATATAATCTATAGTACTACAA |
| | | TAATATCAGTGTTTTAGAGAATCATATAAATGAACAGTTAGACATGGTCTAAAGGACAATTGAGTATTTTGACAACAGGACTCTACAGTTT |
| | | TATCTTTTTAGTGTGCATGTGTTCTCCTTTTTTTTTGCAAATAGCTTCACCTATATAATACTTCATCCATTTTATTAGTACATCCATTTAGGG |
| | | TTTAGGGTTAATGGTTTTTATAGACTAATTTTTTTAGTACATCTATTTTATTCTATTTTAGCCTCTAAATTAAGAAAACTAAAACTCTATTTTA |
| | | GTTTTTTTATTTAATAATTTAGATATAAAATAGAATAAAATAAAGTGACTAAAAATTAAACAAATACCCTTTAAGAAATTAAAAAAACTAAGG |
| | | AAACATTTTTCTTGTTTCGAGTAGATAATGCCAGCCTGTTAAACGCCGTCGACGAGTCTAACGGACACCAACCAGCGAACCAGCAGCGT |
| | | CGCGTCGGGCCAAGCGAAGCAGACGGCACGGCATCTCTGTCGCTGCCTCTGGACCCCTCTCGAGAGTTCCGCTCCACCGTTGGACTT |
| | | GCTCCGCTGTCGGCATCCAGAAATTGCGTGGCGGAGCGGCAGACGTGAGCCGGCACGGCAGGCGGCCTCCTCCTCCTCTCACGGCA |
| | | CCGGCAGCTACGGGGGATTCCTTTCCCACCGCTCCTTCGCTTTCCCTTCCTCGCCCGCCGTAATAAATAGACACCCCCTCCACACCCT |
| | | CTTTCCCCAACCTCGTGTTGTTCGGAGCGCACACACACACAACCAGATCTCCCCCAAATCCACCCGTCGGCACCTCCGCTTCAAGGTA |
| | | CGCCGCTCGTCCTCCCCCCCCCCCCTCTCTACCTTCTCTAGATCGGCGTTCCGGTCCATGGTTAGGGCCCGGTAGTTCTACTTCTGTT |
| | | CATGTTTGTGTTAGATCCGTGTTTGTGTTAGATCCGTGCTGCTAGCGTTCGTACACGGATGCGACCTGTACGTCAGACACGTTCTGATT |
| | | GCTAACTTGCCAGTGTTTCTCTTTGGGGAATCCTGGGATGGCTCTAGCCGTTCCGCAGACGGGATCGATTTCATGATTTTTTTTGTTTCG |
| | | TTGCATAGGGTTTGGTTTGCCCTTTTCCTTTATTTCAATATATGCCGTGCACTTGTTTGTCGGGTCATCTTTTCATGCTTTTTTTTGTCTTG |
| | | GTTGTGATGATGTGGTCTGGTTGGGCGGTCGTTCTAGATCGGAGTAGAATTCTGTTTCAAACTACCTGGTGGATTTATTAATTTTGGATC |
| | | TGTATGTGTGTGCCATACATATTCATAGTTACGAATTGAAGATGATGGATGGAAATATCGATCTAGGATAGGTATACATGTTGATGCGGG |
| | | TTTTACTGATGCATATACAGAGATGCTTTTTGTTCGCTTGGTTGTGATGATGTGGTGTGGTTGGGCGGTCGTTCATTCGTTCTAGATCGG |
| | | AGTAGAATACTGTTTCAAACTACCTGGTGTATTTATTAATTTTGGAACTGTATGTGTGTGTCATACATCTTCATAGTTACGAGTTTAAGAT |
| | | GGATGGAAATATCGATCTAGGATAGGTATACATGTTGATGTGGGTTTTACTGATGCATATACATGATGGCATATGCAGCATCTATTCATA |
| | | TGCTCTAACCTTGAGTACCTATCTATTATAATAAACAAGTATGTTTTATAATTATTTTGATCTTGATATACTTGGATGATGGCATATGCAGC |
| | | AGCTATATGTGGATTTTTTTAGCCCTGCCTTCATACGCTATTTATTTGCTTGGTACTGTTTCTTTTGTCGATGCTCACCCTGTTGTTTGGT |
| | | GTTACTTCTGCAGGGATCCGGCAGCAGCCATGCAGAAGCTGATCAACAGCGTGCAGAACTACGCCTGGGGCAGCAAGACCGCCCTGA |
| | | CCGAGCTGTACGGCATGGAGAACCCCAGCAGCCAGCCCATGGCCGAGCTGTGGATGGGCGCCCACCCCAAGAGCAGCAGCCGCGTG |
| | | CAGAACGCCGCCGGCGACATCGTGAGCCTGCGCGACGTGATCGAGAGCGACAAGAGCACCCTGCTGGGCGAGGCCGTGGCCAAGC |
| | | GCTTCGGCGAGCTGCCCTTCCTGTTCAAGGTGCTGTGCGCCGCCCAGCCCCTGAGCATCCAGGTGCACCCCAACAAGCACAACAGCG |
| | | AGATCGGCTTCGCCAAGGAGAACGCCGCCGGCATCCCCATGGACGCCGCCGAGCGCAACTACAAGGACCCCAACCACAAGCCCGAG |
| | | CTGGTGTTCGCCCTGACCCCCTTCCTGGCCATGAACGCCTTCCGCGAGTTCAGCGAGATCGTGAGCCTGCTGCAGCCCGTGGCCGGC |
| | | GCCCACCCCGCCATCGCCCACTTCCTGCAGCAGCCCGACGCCGAGCGCCTGAGCGAGCTGTTCGCCAGCCTGCTGAACATGCAGGG |
| | | CGAGGAGAAGAGCCGCGCCCTGGCCATCCTGAAGAGCGCCCTGGACAGCCAGCAGGGCGAGCCCTGGCAGACCATCCGCCTGATCA |
| | | GCGAGTTCTACCCCGAGGACAGCGGCCTGTTCAGCCCCCTGCTGCTGAACGTGGTGAAGCTGAACCCCGGCGAGGCCATGTTCCTGT |
| | | TCGCCGAGACCCCCCACGCCTACCTGCAGGGCGTGGCCCTGGAGGTGATGGCCAACAGCGACAACGTGCTGCGCGCCGGCCTGACC |
| | | CCCAAGTACATCGACATCCCCGAGCTGGTGGCCAACGTGAAGTTCGAGGCCAAGCCCGCCAACCAGCTGCTGACCCAGCCCGTGAAG |
| | | CAGGGCGCCGAGCTGGACTTCCCCATCCCCGTGGACGACTTCGCCTTCAGCCTGCACGACCTGAGCGACAAGGAGACCACCATCAGC |
| | | CAGCAGAGCGCCGCCATCCTGTTCTGCGTGGAGGGCGACGCCACCCTGTGGAAGGGCAGCCAGCAGCTGCAGCTGAAGCCCGGCGA |
| | | GAGCGCCTTCATCGCCGCCAACGAGAGCCCCGTGACCGTGAAGGGCCACGGCCGCCTGGCCCGCGTGTACAACAAGCTGTGATAGG |
| | | AGCTCGATCCGTCGACCTGCAGATCGTTCAAACATTTGGCAATAAAGTTTCTTAAGATTGAATCCTGTTGCCGGTCTTGCGATGATTATC |
| | | ATATAATTTCTGTTGAATTACGTTAAGCATGTAATAATTAACATGTAATGCATGACGTTATTTATGAGATGGGTTTTTATGATTAGAGTCCC |
| | | GCAATTATACATTTAATACGCGATAGAAAACAAAATATAGCGCGCAAACTAGGATAAATTATCGCGCGCGGTGTCATCTATGTTACTAGA |
| | | TCGGCGCGCCGCAATTGAAGTTTGGGCGGCCAGCATGGCCGTATCCGCAATGTGTTATTAAGTTGTCTAAGCGTCAATTTGTTTACACC |
| | | ACAATATATCCTGCCACCAGCCAGCCAACAGCTCCCCGACCGGCAGCTCGGCACAAAATCACCACTCGATACAGGCAGCCCATCAGAA |
| | | TTAATTCTCATGTTTGACAGCTTATCATCGACTGCACGGTGCACCAATGCTTCTGGCGTCAGGCAGCCATCGGAAGCTGTGGTATGGCT |
| | | GTGCAGGTCGTAAATCACTGCATAATTCGTGTCGCTCAAGGCGCACTCCCGTTCTGGATAATGTTTTTTGCGCCGACATCATAACGGTT |
| | | CTGGCAAATATTCTGAAATGAGCTGTTGACAATTAATCATCCGGCTCGTATAATGTGTGGAATTGTGAGCGGATAACAATTTCACACAGG |
| | | AAACAGACCATGAGGGAAGCGTTGATCGCCGAAGTATCGACTCAACTATCAGAGGTAGTTGGCGTCATCGAGCGCCATCTCGAACCGA |
| | | CGTTGCTGGCCGTACATTTGTACGGCTCCGCAGTGGATGGCGGCCTGAAGCCACACAGTGATATTGATTTGCTGGTTACGGTGACCGT |
| | | AAGGCTTGATGAAACAACGCGGCGAGCTTTGATCAACGACCTTTTGGAAACTTCGGCTTCCCCTGGAGAGAGCGAGATTCTCCGCGCT |
| | | GTAGAAGTCACCATTGTTGTGCACGACGACATCATTCCGTGGCGTTATCCAGCTAAGCGCGAACTGCAATTTGGAGAATGGCAGCGCA |
| | | ATGACATTCTTGCAGGTATCTTCGAGCCAGCCACGATCGACATTGATCTGGCTATCTTGCTGACAAAAGCAAGAGAACATAGCGTTGCC |
| | | TTGGTAGGTCCAGCGGCGGAGGAACTCTTTGATCCGGTTCCTGAACAGGATCTATTTGAGGCGCTAAATGAAACCTTAACGCTATGGAA |
| | | CTCGCCGCCCGACTGGGCTGGCGATGAGCGAAATGTAGTGCTTACGTTGTCCCGCATTTGGTACAGCGCAGTAACCGGCAAAATCGC |
| | | GCCGAAGGATGTCGCTGCCGACTGGGCAATGGAGCGCCTGCCGGCCCAGTATCAGCCCGTCATACTTGAAGCTAGGCAGGCTTATCT |
| | | TGGACAAGAAGATCGCTTGGCCTCGCGCGCAGATCAGTTGGAAGAATTTGTTCACTACGTGAAAGGCGAGATCACCAAAGTAGTCGGC |
| | | AAATAAAGCTCTAGTGGATCTCCGTACCCGGGGATCTGGCTCGCGGCGGACGCACGACGCCGGGGCGAGACCATAGGCGATCTCCTA |
| | | AATCAATAGTAGCTGTAACCTCGAAGCGTTTCACTTGTAACAACGATTGAGAATTTTTGTCATAAAATTGAAATACTTGGTTCGCATTTTT |
| | | GTCATCCGCGGTCAGCCGCAATTCTGACGAACTGCCCATTTAGCTGGAGATGATTGTACATCCTTCACGTGAAAATTTCTCAAGCGCTG |
| | | TGAACAAGGGTTCAGATTTTAGATTGAAAGGTGAGCCGTTGAAACACGTTCTTCTTGTCGATGACGACGTCGCTATGCGGCATCTTATT |
| | | ATTGAATACCTTACGATCCACGCCTTCAAAGTGACCGCGGTAGCCGACAGCACCCAGTTCACAAGAGTACTCTCTTCCGCGACGGTCG |
| | | ATGTCGTGGTTGTTGATCTAGATTTAGGTCGTGAAGATGGGCTCGAGCTAGGAGCAAGTGATTTTATCGCTAAGCCGTTCAGTATCAGA |
| | | GAGTTTCTAGCACGCATTCGGGTTGCCTTGCGCGTGCGCCCCAACGTTGTCCGCTCCAAAGACCGACGGTCTTTTTGTTTTACTGACTG |
| | | GACACTTAATCTCAGGCAACGTCGCTTGATGTCCGAAGCTGGCGGTGAGGTGAAACTTACGGCAGGTGAGTTCAATCTTCTCCTCGCG |
| | | TTTTTAGAGAAACCCCGCGACGTTCTATCGCGCGAGCAACTTCTCATTGCCAGTCGAGTACGCGACGAGGAGGTTTATGACAGGAGTA |
| | | TAGATGTTCTCATTTTGAGGCTGCGCCGCAAACTTGAGGCAGATCCGTCAAGCCCTCAACTGATAAAAACAGCAAGAGGTGCCGGTTAT |
| | | TTCTTTGACGCGGACGTGCAGGTTTCGCACGGGGGGACGATGGCAGCCTGAGCCAATTCCCAGATCCCCGAGGAATCGGCGTGAGC |
| | | GGTCGCAAACCATCCGGCCCGGTACAAATCGGCGCGGCGCTGGGTGATGACCTGGTGGAGAAGTTGAAGGCCGCGCAGGCCGCCCA |
| | | GCGGCAACGCATCGAGGCAGAAGCACGCCCCGGTGAATCGTGGCAAGCGGCCGCTGATCGAATCCGCAAAGAATCCCGGCAACCGC |
| | | CGGCAGCCGGTGCGCCGTCGATTAGGAAGCCGCCCAAGGGCGACGAGCAACCAGATTTTTTCGTTCCGATGCTCTATGACGTGGGCA |
| | | CCCGCGATAGTCGCAGCATCATGGACGTGGCCGTTTTCCGTCTGTCGAAGCGTGACCGACGAGCTGGCGAGGTGATCCGCTACGAGC |
| | | TTCCAGACGGGCACGTAGAGGTTTCCGCAGGGCCGGCCGGCATGGCCAGTGTGTGGGATTACGACCTGGTACTGATGGCGGTTTCCC |
| | | ATCTAACCGAATCCATGAACCGATACCGGGAAGGGAAGGGAGACAAGCCCGGCCGCGTGTTCCGTCCACACGTTGCGGACGTACTCA |
| | | AGTTCTGCCGGCGAGCCGATGGCGGAAAGCAGAAAGACGACCTGGTAGAAACCTGCATTCGGTTAAACACCACGCACGTTGCCATGC |
| | | AGCGTACGAAGAAGGCCAAGAACGGCCGCCTGGTGACGGTATCCGAGGGTGAAGCCTTGATTAGCCGCTACAAGATCGTAAAGAGCG |
| | | AAACCGGGCGGCCGGAGTACATCGAGATCGAGCTAGCTGATTGGATGTACCGCGAGATCACAGAAGGCAAGAACCCGGACGTGCTGA |
| | | CGGTTCACCCCGATTACTTTTTGATCGATCCCGGCATCGGCCGTTTTCTCTACCGCCTGGCACGCCGCGCCGCAGGCAAGGCAGAAG |
| | | CCAGATGGTTGTTCAAGACGATCTACGAACGCAGTGGCAGCGCCGGAGAGTTCAAGAAGTTCTGTTTCACCGTGCGCAAGCTGATCGG |
| | | GTCAAATGACCTGCCGGAGTACGATTTGAAGGAGGAGGCGGGGCAGGCTGGCCCGATCCTAGTCATGCGCTACCGCAACCTGATCGA |
| | | GGGCGAAGCATCCGCCGGTTCCTAATGTACGGAGCAGATGCTAGGGCAAATTGCCCTAGCAGGGGAAAAAGGTCGAAAAGGTCTCTT |
| | | TCCTGTGGATAGCACGTACATTGGGAACCCAAAGCCGTACATTGGGAACCGGAACCCGTACATTGGGAACCCAAAGCCGTACATTGGG |
| | | AACCGGTCACACATGTAAGTGACTGATATAAAAGAGAAAAAAGGCGATTTTTCCGCCTAAAACTCTTTAAAACTTATTAAAACTCTTAAAA |
| | | CCCGCCTGGCCTGTGCATAACTGTCTGGCCAGCGCACAGCCGAAGAGCTGCAAAAAGCGCCTACCCTTCGGTCGCTGCGCTCCCTAC |
| | | GCCCCGCCGCTTCGCGTCGGCCTATCGCGGCCGCTGGCCGCTCAAAAATGGCTGGCCTACGGCCAGGCAATCTACCAGGGCGCGGA |
| | | CAAGCCGCGCCGTCGCCACTCGACCGCCGGCGCTGAGGTCTGCCTCGTGAAGAAGGTGTTGCTGACTCATACCAGGCCTGAATCGCC |
| | | CCATCATCCAGCCAGAAAGTGAGGGAGCCACGGTTGATGAGAGCTTTGTTGTAGGTGGACCAGTTGGTGATTTTGAACTTTTGCTTTGC |
| | | CACGGAACGGTCTGCGTTGTCGGGAAGATGCGTGATCTGATCCTTCAACTCAGCAAAAGTTCGATTTATTCAACAAAGCCGCCGTCCC |
| | | GTCAAGTCAGCGTAATGCTCTGCCAGTGTTACAACCAATTAACCAATTCTGATTAGAAAAACTCATCGAGCATCAAATGAAACTGCAATT |
| | | TATTCATATCAGGATTATCAATACCATATTTTTGAAAAAGCCGTTTCTGTAATGAAGGAGAAAACTCACCGAGGCAGTTCCATAGGATGG |
| | | CAAGATCCTGGTATCGGTCTGCGATTCCGACTCGTCCAACATCAATACAACCTATTAATTTCCCCTCGTCAAAAATAAGGTTATCAAGTG |
| | | AGAAATCACCATGAGTGACGACTGAATCCGGTGAGAATGGCAAAAGCTCTGCATTAATGAATCGGCCAACGCGCGGGGAGAGGCGGT |
| | | TTGCGTATTGGGCGCTCTTCCGCTTCCTCGCTCACTGACTCGCTGCGCTCGGTCGTTCGGCTGCGGCGAGCGGTATCAGCTCACTCAA |
| | | AGGCGGTAATACGGTTATCCACAGAATCAGGGGATAACGCAGGAAAGAACATGTGAGCAAAAGGCCAGCAAAAGGCCAGGAACCGTA |
| | | AAAAGGCCGCGTTGCTGGCGTTTTTCCATAGGCTCCGCCCCCCTGACGAGCATCACAAAAATCGACGCTCAAGTCAGAGGTGGCGAAA |
| | | CCCGACAGGACTATAAAGATACCAGGCGTTTCCCCCTGGAAGCTCCCTCGTGCGCTCTCCTGTTCCGACCCTGCCGCTTACCGGATAC |
| | | CTGTCCGCCTTTCTCCCTTCGGGAAGCGTGGCGCTTTCTCATAGCTCACGCTGTAGGTATCTCAGTTCGGTGTAGGTCGTTCGCTCCAA |
| | | GCTGGGCTGTGTGCACGAACCCCCCGTTCAGCCCGACCGCTGCGCCTTATCCGGTAACTATCGTCTTGAGTCCAACCCGGTAAGACA |
| | | CGACTTATCGCCACTGGCAGCAGCCACTGGTAACAGGATTAGCAGAGCGAGGTATGTAGGCGGTGCTACAGAGTTCTTGAAGTGGTG |
| | | GCCTAACTACGGCTACACTAGAAGAACAGTATTTGGTATCTGCGCTCTGCTGAAGCCAGTTACCTTCGGAAAAAGAGTTGGTAGCTCTT |
| | | GATCCGGCAAACAAACCACCGCTGGTAGCGGTGGTTTTTTTGTTTGCAAGCAGCAGATTACGCGCAGAAAAAAAGGATCTCAAGAAGAT |
| | | CCTTTGATCTTTTCTACGGGGTCTGACGCTCAGTGGAACGAAAACTCACGTTAAGGGATTTTGGTCATGAGATTATCAAAAAGGATCTTC |
| | | ACCTAGATCCTTTTGATCCGGACAAACAAACAAATACAGTAATTTAGCCAGGACGTCGGCCGAAAGAGCGACAAGCAGATCACGCTTTT |
| | | CGACAGCGTCGGATTTGCGATCGAGGATTTTTCGGCGCTGCGCTACGTCCGCGACCGCGTTGAGGGATCAAGCCACAGCAGCCCACT |
| | | CGACCTTCTAGCCGACCCAGACGAGCCAAGGGATCTTTTTGGAATGCTGCTCCGTCGTCAGGCTTTCCGACGTTTGGGTGGTTGAACA |
| | | GAAGTCATTATCGCACGGAATGCCAAGCACTCCCGAGGGG |
| 314 | CPF1 SUNFLOWER | AACCCTGTGGTTGGCATGCACATACAAATGGACGAACGGATAAACCTTTTCACGCCCTTTTAAATATCCGATTATTCTAATAAACGCTCT |
| | EXPRESSION VECTOR | TTTCTCTTAGGTTTACCCGCCAATATATCCTGTCAAACACTGATAGTTTAAACTGGCACTAGCCTAACGGTGTTGACTAACTAGGCCGCT |
| | (SEQ ID NO: 314) | TCCCTAATTAGCTAAGGTACCGGCGCGCCACCGGTGAGCTCGGGACCCGAATTCATTATGTGGTCTAGGTAGGTTCTATATATAAGAAA |
| | | ACTTGAAATGTTCTAAAAAAAAATTCAAGCCCATGCATGATTGAAGCAAACGGTATAGCAACGGTGTTAACCTGATCTAGTGATCTCTTG |
| | | CAATCCTTAACGGCCACCTACCGCAGGTAGCAAACGGCGTCCCCCTCCTCGATATCTCCGCGGCGACCTCTGGCTTTTTCCGCGGAAT |
| | | TGCGCGGTGGGGACGGATTCCACGAGACCGCGACGCAACCGCCTCTCGCCGCTGGGCCCCACACCGCTCGGTGCCGTAGCCTCACG |
| | | GGACTCTTTCTCCCTCCTCCCCCGTTATAAATTGGCTTCATCCCCTCCTTGCCTCATCCATCCAAATCCCAGTCCCCAATCCCATCCCTT |
| | | CGTAGGAGAAATTCATCGAAGCTAAGCGAATCCTCGCGATCCTCTCAAGGTACTGCGAGTTTTCGATCCCCCTCTCGACCCCTCGTATG |
| | | TTTGTGTTTGTCGTAGCGTTTGATTAGGTATGCTTTCCCTGTTTGTGTTCGTCGTAGCGTTTGATTAGGTATGCTTTCCCTGTTCGTGTTC |
| | | ATCGTAGTGTTTGATTAGGTCGTGTGAGGCGATGGCCTGCTCGCGTCCTTCGATCTGTAGTCGATTTGCGGGTCGTGGTGTAGATCTG |
| | | CGGGCTGTGATGAAGTTATTTGGTGTGATCTGCTCGCCTGATTCTGCGGGTTGGCTCGAGTAGATATGATGGTTGGACCGGTTGGTTC |
| | | GTTTACCGCGCTAGGGTTGGGCTGGGATGATGTTGCATGCGCCGTTGCGCGTGATCCCGCAGCAGGACTTGCGTTTGATTGCCAGAT |
| | | CTCGTTACGATTATGTGATTTGGTTTGGACTTTTTAGATCTGTAGCTTCTGCTTATGTGCCAGATGCGCCTACTGCTCATATGCCTGATG |
| | | ATAATCATAAATGGCTGTGGAACTAACTAGTTGATTGCGGAGTCATGTATCAGCTACAGGTGTAGGGACTAGCTACAGGTGTAGGGACT |
| | | TGCGTCTAATTGTTTGGTCCTTTACTCATGTTGCAATTATGCAATTTAGTTTAGATTGTTTGTTCCACTCATCTAGGCTGTAAAAGGGACA |
| | | CTGCTTAGATTGCTGTTTAATCTTTTTAGTAGATTATATTATATTGGTAACTTATTACCCCTATTACATGCCATACGTGACTTCTGCTCATG |
| | | CCTGATGATAATCATAGATCACTGTGGAATTAATTAGTTGATTGTTGAATCATGTTTCATGTACATACCACGGCACAATTGCTTAGTTCCT |
| | | TAACAAATGCAAATTTTACTGATCCATGTATGATTTGCGTGGTTCTCTAATGTGAAATACTATAGCTACTTGTTAGTAAGAATCAGGTTCG |
| | | TATGCTTAATGCTGTATGTGCCTTCTGCTCATGCCTGATGATAATCATATATCACTGGAATTAATTAGTTGATCGTTTAATCATATATCAAG |
| | | TACATACCATGCCACAATTTTTAGTCACTTAACCCATGCAGATTGAACTGGTCCCTGCATGTTTTGCTAAATTGTTCTATTCTGATTAGAC |
| | | CATATATCATGTATTTTTTTTTGGTAATGGTTCTCTTATTTTAAATGCTATATAGTTCTGGTACTTGTTAGAAAGATCTGCTTCATAGTTTAG |
| | | TTGCCTATCCCTCGAATTAGGATGCTGAGCAGCTGATCCTATAGCTTTGTTTCATGTATCAATTCTTTTGTGTTCAACAGTCAGTTTTTGT |
| | | TAGATTCATTGTAACTTATGGTCGCTTACTCTTCTGGTCCTCAATGCTTGCAGGGATCCCCTAAATAGACCATGGCTCCTAAGAAGAAGC |
| | | GGAAGGTTGGTATTCACGGGGTGCCTGCGGCTTCAAAGCTCGAGAAATTCACCAACTGTTATTCGTTGAGCAAAACACTGCGGTTTAAA |
| | | GCGATTCCAGTCGGCAAGACTCAAGAGAATATAGACAATAAGCGGCTGTTGGTGGAAGATGAAAAGCGCGCGGAAGACTACAAAGGG |
| | | GTGAAGAAGTTGTTGGACAGATACTACCTCTCTTTTATCAATGATGTCTTGCACTCAATCAAATTGAAGAATCTGAACAACTACATCTCCC |
| | | TCTTCAGAAAGAAAACAAGGACAGAAAAGGAGAATAAGGAACTTGAAAATTTGGAGATCAATCTGAGGAAAGAGATCGCGAAAGCCTTT |
| | | AAAGGCAACGAAGGATACAAAAGTCTGTTCAAGAAGGATATAATTGAGACAATTTTGCCAGAGTTCCTCGATGACAAGGACGAGATTGC |
| | | GCTGGTCAATTCGTTCAACGGATTCACAACAGCATTCACAGGCTTCTTTGATAATCGGGAAAATATGTTCTCTGAGGAGGCAAAGTCCA |
| | | CTTCTATTGCGTTCAGGTGTATCAATGAGAATCTCACTAGGTACATTTCCAACATGGATATCTTTGAGAAGGTTGACGCAATTTTTGACAA |
| | | GCACGAAGTTCAGGAGATTAAGGAGAAGATCCTCAATTCCGATTATGACGTTGAGGACTTCTTCGAGGGTGAGTTTTTTAATTTCGTGCT |
| | | CACTCAAGAGGGTATCGACGTGTATAATGCGATCATCGGTGGGTTCGTGACTGAGTCCGGTGAAAAGATTAAGGGATTGAACGAGTAT |
| | | ATCAACCTTTACAACCAAAAGACGAAACAGAAGCTGCCAAAGTTCAAGCCTCTTTACAAACAGGTTCTTTCAGACCGCGAGTCACTCTC |
| | | GTTCTATGGGGAGGGCTACACTTCGGATGAGGAAGTCCTGGAGGTGTTCAGGAATACTCTCAATAAGAATTCGGAGATTTTCTCTTCTA |
| | | TAAAAAAACTGGAAAAGTTGTTTAAGAATTTTGACGAATACTCTAGCGCCGGCATATTTGTGAAAAACGGCCCGGCCATATCAACGATAA |
| | | GTAAAGATATCTTCGGCGAATGGAACGTGATCAGAGACAAATGGAACGCGGAGTATGACGATATTCACCTGAAGAAGAAGGCTGTCGT |
| | | AACGGAGAAGTACGAGGATGATCGCAGGAAAAGCTTCAAAAAGATCGGAAGTTTCAGCCTGGAACAGTTGCAGGAGTATGCTGACGCC |
| | | GATCTTAGCGTCGTCGAGAAGTTGAAGGAGATAATCATCCAAAAGGTCGACGAGATATATAAAGTCTATGGATCAAGTGAAAAACTGTT |
| | | CGACGCCGACTTCGTTTTGGAGAAGTCCCTGAAGAAGAACGACGCTGTTGTTGCCATTATGAAGGATCTGCTCGACAGCGTGAAGAGT |
| | | TTCGAGAACTATATTAAGGCTTTTTTCGGGGAGGGGAAGGAGACTAACAGAGATGAGTCCTTCTACGGAGACTTCGTCCTCGCGTACG |
| | | ATATACTCCTTAAGGTAGACCACATCTACGACGCAATCAGAAATTACGTGACACAAAAGCCGTACAGCAAGGACAAGTTCAAACTCTACT |
| | | TCCAGAACCCCCAGTTCATGGGGGGCTGGGACAAGGACAAGGAAACGGATTACAGGGCTACGATCCTGAGGTATGGTTCAAAATACTA |
| | | CTTGGCGATTATGGACAAGAAGTACGCCAAGTGTCTCCAGAAGATTGACAAAGACGATGTCAATGGCAATTATGAGAAGATCAACTACA |
| | | AGCTGCTTCCGGGTCCGAACAAGATGCTCCCAAAGGTTTTCTTCAGCAAGAAATGGATGGCCTACTATAACCCAAGCGAGGACATCCA |
| | | GAAGATTTATAAGAACGGTACGTTCAAGAAGGGCGACATGTTCAATCTTAACGACTGTCACAAGCTGATCGACTTCTTCAAAGACTCAAT |
| | | TAGCCGGTACCCAAAGTGGTCTAACGCCTATGACTTCAACTTTTCGGAAACCGAGAAGTACAAGGATATAGCCGGATTTTATAGAGAGG |
| | | TGGAAGAGCAGGGCTACAAGGTGTCATTCGAGTCCGCCAGCAAGAAGGAAGTGGACAAGCTCGTGGAAGAGGGTAAGCTCTACATGT |
| | | TCCAGATTTATAATAAAGACTTTAGCGATAAGAGCCACGGGACACCTAATCTCCACACAATGTATTTCAAGCTGCTCTTCGACGAGAATA |
| | | ACCACGGCCAAATCAGGTTGTCAGGAGGGGCTGAACTCTTCATGCGGCGCGCTAGCCTTAAGAAGGAGGAGCTTGTAGTCCACCCTG |
| | | CGAATAGTCCAATTGCGAATAAGAACCCGGACAATCCTAAAAAGACTACAACATTGAGCTACGACGTGTACAAGGATAAGAGGTTTTCC |
| | | GAGGATCAGTACGAGCTCCACATCCCGATTGCGATCAACAAGTGCCCAAAGAATATTTTCAAGATAAACACAGAGGTGCGTGTACTCCT |
| | | GAAGCATGACGACAATCCTTACGTCATTGGGATTGATCGGGGCGAGAGGAACCTCCTCTATATTGTGGTGGTGGACGGGAAGGGGAA |
| | | CATAGTCGAACAGTACTCCCTTAACGAAATAATTAACAATTTCAACGGCATCCGTATCAAGACCGACTACCATTCGTTGCTGGACAAGAA |
| | | GGAGAAGGAGAGATTTGAGGCGCGGCAAAATTGGACAAGTATCGAGAACATCAAGGAACTCAAAGCAGGTTATATCTCTCAAGTTGTG |
| | | CATAAGATATGCGAGCTGGTTGAGAAGTATGACGCAGTGATCGCTCTTGAGGACCTCAACTCGGGCTTTAAGAATTCTAGAGTTAAAGT |
| | | GGAGAAGCAGGTCTATCAAAAGTTCGAGAAGATGCTTATAGATAAGCTCAACTACATGGTCGATAAGAAATCGAACCCATGTGCCACCG |
| | | GCGGCGCACTCAAAGGTTACCAAATAACAAACAAATTCGAGTCCTTCAAATCGATGAGTACTCAGAATGGGTTCATATTTTATATACCGG |
| | | CGTGGCTTACGTCTAAGATCGACCCGTCAACTGGTTTTGTCAACCTGTTGAAGACGAAATACACGTCCATTGCCGATTCAAAAAAGTTC |
| | | ATATCTAGTTTTGATCGTATTATGTACGTCCCAGAGGAAGATCTTTTCGAGTTTGCTCTCGACTACAAAAACTTTTCGCGCACCGATGCG |
| | | GATTACATTAAAAAATGGAAACTCTATTCGTACGGCAACAGAATCAGGATTTTTCGCAACCCTAAGAAGAATAACGTCTTTGATTGGGAG |
| | | GAAGTTTGCTTGACTAGCGCGTACAAGGAGCTCTTTAATAAGTATGGCATTAACTACCAACAGGGTGATATCAGAGCACTGCTTTGCGA |
| | | ACAATCTGACAAGGCTTTCTACTCATCCTTCATGGCTTTGATGAGCCTGATGCTCCAGATGAGAAATTCAATTACAGGCAGAACCGACG |
| | | TGGATTTCTTGATCTCCCCGGTTAAAAATTCTGATGGCATCTTTTACGATAGCAGGAACTATGAAGCGCAAGAGAATGCGATTCTGCCAA |
| | | AAAATGCAGACGCCAACGGTGCCTATAACATCGCCAGGAAAGTCCTGTGGGCGATCGGCCAGTTCAAAAAGGCCGAAGACGAAAAATT |
| | | GGACAAGGTCAAAATCGCTATCAGCAACAAAGAGTGGCTGGAGTATGCTCAGACATCCGTAAAGCATAAGCGTCCTGCTGCCACCAAA |
| | | AAGGCCGGACAGGCTAAGAAAAAGAAGTGATTAATTAAGATCGTTCAAACATTTGGCAATAAAGTTTCTTAAGATTGAATCCTGTTGCCG |
| | | GTCTTGCGATGATTATCATATAATTTCTGTTGAATTACGTTAAGCATGTAATAATTAACATGTAATGCATGACGTTATTTATGAGATGGGTT |
| | | TTTATGATTAGAGTCCCGCAATTATACATTTAATACGCGATAGAAAACAAAATATAGCGCGCAAACTAGGATAAATTATCGCGCGCGGTG |
| | | TCATCTATGTTACTAGATCTTCGAAGAATTCATTATGTGGTCTAGGTAGGTTCTATATATAAGAAAACTTGAAATGTTCTAAAAAAAAATTC |
| | | AAGCCCATGCATGATTGAAGCAAACGGTATAGCAACGGTGTTAACCTGATCTAGTGATCTCTTGCAATCCTTAACGGCCACCTACCGCA |
| | | GGTAGCAAACGGCGTCCCCCTCCTCGATATCTCCGCGGCGACCTCTGGCTTTTTCCGCGGAATTGCGCGGTGGGGACGGATTCCACG |
| | | AGACCGCGACGCAACCGCCTCTCGCCGCTGGGCCCCACACCGCTCGGTGCCGTAGCCTCACGGGACTCTTTCTCCCTCCTCCCCCGT |
| | | TATAAATTGGCTTCATCCCCTCCTTGCCTCATCCATCCAAATCCCAGTCCCCAATCCCATCCCTTCGTAGGAGAAATTCATCGAAGCTAA |
| | | GCGAATCCTCGCGATCCTCTCAAGGTACTGCGAGTTTTCGATCCCCCTCTCGACCCCTCGTATGTTTGTGTTTGTCGTAGCGTTTGATT |
| | | AGGTATGCTTTCCCTGTTTGTGTTCGTCGTAGCGTTTGATTAGGTATGCTTTCCCTGTTCGTGTTCATCGTAGTGTTTGATTAGGTCGTG |
| | | TGAGGCGATGGCCTGCTCGCGTCCTTCGATCTGTAGTCGATTTGCGGGTCGTGGTGTAGATCTGCGGGCTGTGATGAAGTTATTTGGT |
| | | GTGATCTGCTCGCCTGATTCTGCGGGTTGGCTCGAGTAGATATGATGGTTGGACCGGTTGGTTCGTTTACCGCGCTAGGGTTGGGCTG |
| | | GGATGATGTTGCATGCGCCGTTGCGCGTGATCCCGCAGCAGGACTTGCGTTTGATTGCCAGATCTCGTTACGATTATGTGATTTGGTTT |
| | | GGACTTTTTAGATCTGTAGCTTCTGCTTATGTGCCAGATGCGCCTACTGCTCATATGCCTGATGATAATCATAAATGGCTGTGGAACTAA |
| | | CTAGTTGATTGCGGAGTCATGTATCAGCTACAGGTGTAGGGACTAGCTACAGGTGTAGGGACTTGCGTCTAATTGTTTGGTCCTTTACT |
| | | CATGTTGCAATTATGCAATTTAGTTTAGATTGTTTGTTCCACTCATCTAGGCTGTAAAAGGGACACTGCTTAGATTGCTGTTTAATCTTTTT |
| | | AGTAGATTATATTATATTGGTAACTTATTACCCCTATTACATGCCATACGTGACTTCTGCTCATGCCTGATGATAATCATAGATCACTGTG |
| | | GAATTAATTAGTTGATTGTTGAATCATGTTTCATGTACATACCACGGCACAATTGCTTAGTTCCTTAACAAATGCAAATTTTACTGATCCAT |
| | | GTATGATTTGCGTGGTTCTCTAATGTGAAATACTATAGCTACTTGTTAGTAAGAATCAGGTTCGTATGCTTAATGCTGTATGTGCCTTCTG |
| | | CTCATGCCTGATGATAATCATATATCACTGGAATTAATTAGTTGATCGTTTAATCATATATCAAGTACATACCATGCCACAATTTTTAGTCA |
| | | CTTAACCCATGCAGATTGAACTGGTCCCTGCATGTTTTGCTAAATTGTTCTATTCTGATTAGACCATATATCATGTATTTTTTTTTGGTAAT |
| | | GGTTCTCTTATTTTAAATGCTATATAGTTCTGGTACTTGTTAGAAAGATCTGCTTCATAGTTTAGTTGCCTATCCCTCGAATTAGGATGCT |
| | | GAGCAGCTGATCCTATAGCTTTGTTTCATGTATCAATTCTTTTGTGTTCAACAGTCAGTTTTTGTTAGATTCATTGTAACTTATGGTCGCTT |
| | | ACTCTTCTGGTCCTCAATGCTTGCAGGGGATCCAAATTACTGATGAGTCCGTGAGGACGAAACGAGTAAGCTCGTCTAATTTCTACTAA |
| | | GTGTAGATATGCTTGTGCAAGCTGGTGGACTGGCCGGCATGGTCCCAGCCTCCTCGCTGGCGCCGGCTGGGCAACATGCTTCGGCAT |
| | | GGCGAATGGGACGATCGTTCAAACATTTGGCAATAAAGTTTCTTAAGATTGAATCCTGTTGCCGGTCTTGCGATGATTATCATATAATTT |
| | | CTGTTGAATTACGTTAAGCATGTAATAATTAACATGTAATGCATGACGTTATTTATGAGATGGGTTTTTATGATTAGAGTCCCGCAATTAT |
| | | ACATTTAATACGCGATAGAAAACAAAATATAGCGCGCAAACTAGGATAAATTATCGCGCGCGGTGTCATCTATGTTACTAGATCCGGAC |
| | | CGCGCCTGCAGTGCAGCGTGACCCGGTCGTGCCCCTCTCTAGAGATAATGAGCATTGCATGTCTAAGTTATAAAAAATTACCACATATT |
| | | TTTTTTGTCACACTTGTTTGAAGTGCAGTTTATCTATCTTTATACATATATTTAAACTTTACTCTACGAATAATATAATCTATAGTACTACAA |
| | | TAATATCAGTGTTTTAGAGAATCATATAAATGAACAGTTAGACATGGTCTAAAGGACAATTGAGTATTTTGACAACAGGACTCTACAGTTT |
| | | TATCTTTTTAGTGTGCATGTGTTCTCCTTTTTTTTTGCAAATAGCTTCACCTATATAATACTTCATCCATTTTATTAGTACATCCATTTAGGG |
| | | TTTAGGGTTAATGGTTTTTATAGACTAATTTTTTTAGTACATCTATTTTATTCTATTTTAGCCTCTAAATTAAGAAAACTAAAACTCTATTTTA |
| | | GTTTTTTTATTTAATAATTTAGATATAAAATAGAATAAAATAAAGTGACTAAAAATTAAACAAATACCCTTTAAGAAATTAAAAAAACTAAGG |
| | | AAACATTTTTCTTGTTTCGAGTAGATAATGCCAGCCTGTTAAACGCCGTCGACGAGTCTAACGGACACCAACCAGCGAACCAGCAGCGT |
| | | CGCGTCGGGCCAAGCGAAGCAGACGGCACGGCATCTCTGTCGCTGCCTCTGGACCCCTCTCGAGAGTTCCGCTCCACCGTTGGACTT |
| | | GCTCCGCTGTCGGCATCCAGAAATTGCGTGGCGGAGCGGCAGACGTGAGCCGGCACGGCAGGCGGCCTCCTCCTCCTCTCACGGCA |
| | | CCGGCAGCTACGGGGGATTCCTTTCCCACCGCTCCTTCGCTTTCCCTTCCTCGCCCGCCGTAATAAATAGACACCCCCTCCACACCCT |
| | | CTTTCCCCAACCTCGTGTTGTTCGGAGCGCACACACACACAACCAGATCTCCCCCAAATCCACCCGTCGGCACCTCCGCTTCAAGGTA |
| | | CGCCGCTCGTCCTCCCCCCCCCCCCTCTCTACCTTCTCTAGATCGGCGTTCCGGTCCATGGTTAGGGCCCGGTAGTTCTACTTCTGTT |
| | | CATGTTTGTGTTAGATCCGTGTTTGTGTTAGATCCGTGCTGCTAGCGTTCGTACACGGATGCGACCTGTACGTCAGACACGTTCTGATT |
| | | GCTAACTTGCCAGTGTTTCTCTTTGGGGAATCCTGGGATGGCTCTAGCCGTTCCGCAGACGGGATCGATTTCATGATTTTTTTTGTTTCG |
| | | TTGCATAGGGTTTGGTTTGCCCTTTTCCTTTATTTCAATATATGCCGTGCACTTGTTTGTCGGGTCATCTTTTCATGCTTTTTTTTGTCTTG |
| | | GTTGTGATGATGTGGTCTGGTTGGGGGGTCGTTCTAGATCGGAGTAGAATTCTGTTTCAAACTACCTGGTGGATTTATTAATTTTGGATC |
| | | TGTATGTGTGTGCCATACATATTCATAGTTACGAATTGAAGATGATGGATGGAAATATCGATCTAGGATAGGTATACATGTTGATGCGGG |
| | | TTTTACTGATGCATATACAGAGATGCTTTTTGTTCGCTTGGTTGTGATGATGTGGTGTGGTTGGGCGGTCGTTCATTCGTTCTAGATCGG |
| | | AGTAGAATACTGTTTCAAACTACCTGGTGTATTTATTAATTTTGGAACTGTATGTGTGTGTCATACATCTTCATAGTTACGAGTTTAAGAT |
| | | GGATGGAAATATCGATCTAGGATAGGTATACATGTTGATGTGGGTTTTACTGATGCATATACATGATGGCATATGCAGCATCTATTCATA |
| | | TGCTCTAACCTTGAGTACCTATCTATTATAATAAACAAGTATGTTTTATAATTATTTTGATCTTGATATACTTGGATGATGGCATATGCAGC |
| | | AGCTATATGTGGATTTTTTTAGCCCTGCCTTCATACGCTATTTATTTGCTTGGTACTGTTTCTTTTGTCGATGCTCACCCTGTTGTTTGGT |
| | | GTTACTTCTGCAGGGATCCGGCAGCAGCCATGCAGAAGCTGATCAACAGCGTGCAGAACTACGCCTGGGGCAGCAAGACCGCCCTGA |
| | | CCGAGCTGTACGGCATGGAGAACCCCAGCAGCCAGCCCATGGCCGAGCTGTGGATGGGCGCCCACCCCAAGAGCAGCAGCCGCGTG |
| | | CAGAACGCCGCCGGCGACATCGTGAGCCTGCGCGACGTGATCGAGAGCGACAAGAGCACCCTGCTGGGCGAGGCCGTGGCCAAGC |
| | | GCTTCGGCGAGCTGCCCTTCCTGTTCAAGGTGCTGTGCGCCGCCCAGCCCCTGAGCATCCAGGTGCACCCCAACAAGCACAACAGCG |
| | | AGATCGGCTTCGCCAAGGAGAACGCCGCCGGCATCCCCATGGACGCCGCCGAGCGCAACTACAAGGACCCCAACCACAAGCCCGAG |
| | | CTGGTGTTCGCCCTGACCCCCTTCCTGGCCATGAACGCCTTCCGCGAGTTCAGCGAGATCGTGAGCCTGCTGCAGCCCGTGGCCGGC |
| | | GCCCACCCCGCCATCGCCCACTTCCTGCAGCAGCCCGACGCCGAGCGCCTGAGCGAGCTGTTCGCCAGCCTGCTGAACATGCAGGG |
| | | CGAGGAGAAGAGCCGCGCCCTGGCCATCCTGAAGAGCGCCCTGGACAGCCAGCAGGGCGAGCCCTGGCAGACCATCCGCCTGATCA |
| | | GCGAGTTCTACCCCGAGGACAGCGGCCTGTTCAGCCCCCTGCTGCTGAACGTGGTGAAGCTGAACCCCGGCGAGGCCATGTTCCTGT |
| | | TCGCCGAGACCCCCCACGCCTACCTGCAGGGCGTGGCCCTGGAGGTGATGGCCAACAGCGACAACGTGCTGCGCGCCGGCCTGACC |
| | | CCCAAGTACATCGACATCCCCGAGCTGGTGGCCAACGTGAAGTTCGAGGCCAAGCCCGCCAACCAGCTGCTGACCCAGCCCGTGAAG |
| | | CAGGGCGCCGAGCTGGACTTCCCCATCCCCGTGGACGACTTCGCCTTCAGCCTGCACGACCTGAGCGACAAGGAGACCACCATCAGC |
| | | CAGCAGAGCGCCGCCATCCTGTTCTGCGTGGAGGGCGACGCCACCCTGTGGAAGGGCAGCCAGCAGCTGCAGCTGAAGCCCGGCGA |
| | | GAGCGCCTTCATCGCCGCCAACGAGAGCCCCGTGACCGTGAAGGGCCACGGCCGCCTGGCCCGCGTGTACAACAAGCTGTGATAGG |
| | | AGCTCGATCCGTCGACCTGCAGATCGTTCAAACATTTGGCAATAAAGTTTCTTAAGATTGAATCCTGTTGCCGGTCTTGCGATGATTATC |
| | | ATATAATTTCTGTTGAATTACGTTAAGCATGTAATAATTAACATGTAATGCATGACGTTATTTATGAGATGGGTTTTTATGATTAGAGTCCC |
| | | GCAATTATACATTTAATACGCGATAGAAAACAAAATATAGCGCGCAAACTAGGATAAATTATCGCGCGCGGTGTCATCTATGTTACTAGA |
| | | TCGGCGCGCCGCAATTGAAGTTTGGGGGGCCAGCATGGCCGTATCCGCAATGTGTTATTAAGTTGTCTAAGCGTCAATTTGTTTACACC |
| | | ACAATATATCCTGCCACCAGCCAGCCAACAGCTCCCCGACCGGCAGCTCGGCACAAAATCACCACTCGATACAGGCAGCCCATCAGAA |
| | | TTAATTCTCATGTTTGACAGCTTATCATCGACTGCACGGTGCACCAATGCTTCTGGCGTCAGGCAGCCATCGGAAGCTGTGGTATGGCT |
| | | GTGCAGGTCGTAAATCACTGCATAATTCGTGTCGCTCAAGGCGCACTCCCGTTCTGGATAATGTTTTTTGCGCCGACATCATAACGGTT |
| | | CTGGCAAATATTCTGAAATGAGCTGTTGACAATTAATCATCCGGCTCGTATAATGTGTGGAATTGTGAGCGGATAACAATTTCACACAGG |
| | | AAACAGACCATGAGGGAAGCGTTGATCGCCGAAGTATCGACTCAACTATCAGAGGTAGTTGGCGTCATCGAGCGCCATCTCGAACCGA |
| | | CGTTGCTGGCCGTACATTTGTACGGCTCCGCAGTGGATGGCGGCCTGAAGCCACACAGTGATATTGATTTGCTGGTTACGGTGACCGT |
| | | AAGGCTTGATGAAACAACGCGGCGAGCTTTGATCAACGACCTTTTGGAAACTTCGGCTTCCCCTGGAGAGAGCGAGATTCTCCGCGCT |
| | | GTAGAAGTCACCATTGTTGTGCACGACGACATCATTCCGTGGCGTTATCCAGCTAAGCGCGAACTGCAATTTGGAGAATGGCAGCGCA |
| | | ATGACATTCTTGCAGGTATCTTCGAGCCAGCCACGATCGACATTGATCTGGCTATCTTGCTGACAAAAGCAAGAGAACATAGCGTTGCC |
| | | TTGGTAGGTCCAGCGGCGGAGGAACTCTTTGATCCGGTTCCTGAACAGGATCTATTTGAGGCGCTAAATGAAACCTTAACGCTATGGAA |
| | | CTCGCCGCCCGACTGGGCTGGCGATGAGCGAAATGTAGTGCTTACGTTGTCCCGCATTTGGTACAGCGCAGTAACCGGCAAAATCGC |
| | | GCCGAAGGATGTCGCTGCCGACTGGGCAATGGAGCGCCTGCCGGCCCAGTATCAGCCCGTCATACTTGAAGCTAGGCAGGCTTATCT |
| | | TGGACAAGAAGATCGCTTGGCCTCGCGCGCAGATCAGTTGGAAGAATTTGTTCACTACGTGAAAGGCGAGATCACCAAAGTAGTCGGC |
| | | AAATAAAGCTCTAGTGGATCTCCGTACCCGGGGATCTGGCTCGCGGCGGACGCACGACGCCGGGGCGAGACCATAGGCGATCTCCTA |
| | | AATCAATAGTAGCTGTAACCTCGAAGCGTTTCACTTGTAACAACGATTGAGAATTTTTGTCATAAAATTGAAATACTTGGTTCGCATTTTT |
| | | GTCATCCGCGGTCAGCCGCAATTCTGACGAACTGCCCATTTAGCTGGAGATGATTGTACATCCTTCACGTGAAAATTTCTCAAGCGCTG |
| | | TGAACAAGGGTTCAGATTTTAGATTGAAAGGTGAGCCGTTGAAACACGTTCTTCTTGTCGATGACGACGTCGCTATGCGGCATCTTATT |
| | | ATTGAATACCTTACGATCCACGCCTTCAAAGTGACCGCGGTAGCCGACAGCACCCAGTTCACAAGAGTACTCTCTTCCGCGACGGTCG |
| | | ATGTCGTGGTTGTTGATCTAGATTTAGGTCGTGAAGATGGGCTCGAGCTAGGAGCAAGTGATTTTATCGCTAAGCCGTTCAGTATCAGA |
| | | GAGTTTCTAGCACGCATTCGGGTTGCCTTGCGCGTGCGCCCCAACGTTGTCCGCTCCAAAGACCGACGGTCTTTTTGTTTTACTGACTG |
| | | GACACTTAATCTCAGGCAACGTCGCTTGATGTCCGAAGCTGGCGGTGAGGTGAAACTTACGGCAGGTGAGTTCAATCTTCTCCTCGCG |
| | | TTTTTAGAGAAACCCCGCGACGTTCTATCGCGCGAGCAACTTCTCATTGCCAGTCGAGTACGCGACGAGGAGGTTTATGACAGGAGTA |
| | | TAGATGTTCTCATTTTGAGGCTGCGCCGCAAACTTGAGGCAGATCCGTCAAGCCCTCAACTGATAAAAACAGCAAGAGGTGCCGGTTAT |
| | | TTCTTTGACGCGGACGTGCAGGTTTCGCACGGGGGGACGATGGCAGCCTGAGCCAATTCCCAGATCCCCGAGGAATCGGCGTGAGC |
| | | GGTCGCAAACCATCCGGCCCGGTACAAATCGGCGCGGCGCTGGGTGATGACCTGGTGGAGAAGTTGAAGGCCGCGCAGGCCGCCCA |
| | | GCGGCAACGCATCGAGGCAGAAGCACGCCCCGGTGAATCGTGGCAAGCGGCCGCTGATCGAATCCGCAAAGAATCCCGGCAACCGC |
| | | CGGCAGCCGGTGCGCCGTCGATTAGGAAGCCGCCCAAGGGCGACGAGCAACCAGATTTTTTCGTTCCGATGCTCTATGACGTGGGCA |
| | | CCCGCGATAGTCGCAGCATCATGGACGTGGCCGTTTTCCGTCTGTCGAAGCGTGACCGACGAGCTGGCGAGGTGATCCGCTACGAGC |
| | | TTCCAGACGGGCACGTAGAGGTTTCCGCAGGGCCGGCCGGCATGGCCAGTGTGTGGGATTACGACCTGGTACTGATGGCGGTTTCCC |
| | | ATCTAACCGAATCCATGAACCGATACCGGGAAGGGAAGGGAGACAAGCCCGGCCGCGTGTTCCGTCCACACGTTGCGGACGTACTCA |
| | | AGTTCTGCCGGCGAGCCGATGGCGGAAAGCAGAAAGACGACCTGGTAGAAACCTGCATTCGGTTAAACACCACGCACGTTGCCATGC |
| | | AGCGTACGAAGAAGGCCAAGAACGGCCGCCTGGTGACGGTATCCGAGGGTGAAGCCTTGATTAGCCGCTACAAGATCGTAAAGAGCG |
| | | AAACCGGGCGGCCGGAGTACATCGAGATCGAGCTAGCTGATTGGATGTACCGCGAGATCACAGAAGGCAAGAACCCGGACGTGCTGA |
| | | CGGTTCACCCCGATTACTTTTTGATCGATCCCGGCATCGGCCGTTTTCTCTACCGCCTGGCACGCCGCGCCGCAGGCAAGGCAGAAG |
| | | CCAGATGGTTGTTCAAGACGATCTACGAACGCAGTGGCAGCGCCGGAGAGTTCAAGAAGTTCTGTTTCACCGTGCGCAAGCTGATCGG |
| | | GTCAAATGACCTGCCGGAGTACGATTTGAAGGAGGAGGCGGGGCAGGCTGGCCCGATCCTAGTCATGCGCTACCGCAACCTGATCGA |
| | | GGGCGAAGCATCCGCCGGTTCCTAATGTACGGAGCAGATGCTAGGGCAAATTGCCCTAGCAGGGGAAAAAGGTCGAAAAGGTCTCTT |
| | | TCCTGTGGATAGCACGTACATTGGGAACCCAAAGCCGTACATTGGGAACCGGAACCCGTACATTGGGAACCCAAAGCCGTACATTGGG |
| | | AACCGGTCACACATGTAAGTGACTGATATAAAAGAGAAAAAAGGCGATTTTTCCGCCTAAAACTCTTTAAAACTTATTAAAACTCTTAAAA |
| | | CCCGCCTGGCCTGTGCATAACTGTCTGGCCAGCGCACAGCCGAAGAGCTGCAAAAAGCGCCTACCCTTCGGTCGCTGCGCTCCCTAC |
| | | GCCCCGCCGCTTCGCGTCGGCCTATCGCGGCCGCTGGCCGCTCAAAAATGGCTGGCCTACGGCCAGGCAATCTACCAGGGCGCGGA |
| | | CAAGCCGCGCCGTCGCCACTCGACCGCCGGCGCTGAGGTCTGCCTCGTGAAGAAGGTGTTGCTGACTCATACCAGGCCTGAATCGCC |
| | | CCATCATCCAGCCAGAAAGTGAGGGAGCCACGGTTGATGAGAGCTTTGTTGTAGGTGGACCAGTTGGTGATTTTGAACTTTTGCTTTGC |
| | | CACGGAACGGTCTGCGTTGTCGGGAAGATGCGTGATCTGATCCTTCAACTCAGCAAAAGTTCGATTTATTCAACAAAGCCGCCGTCCC |
| | | GTCAAGTCAGCGTAATGCTCTGCCAGTGTTACAACCAATTAACCAATTCTGATTAGAAAAACTCATCGAGCATCAAATGAAACTGCAATT |
| | | TATTCATATCAGGATTATCAATACCATATTTTTGAAAAAGCCGTTTCTGTAATGAAGGAGAAAACTCACCGAGGCAGTTCCATAGGATGG |
| | | CAAGATCCTGGTATCGGTCTGCGATTCCGACTCGTCCAACATCAATACAACCTATTAATTTCCCCTCGTCAAAAATAAGGTTATCAAGTG |
| | | AGAAATCACCATGAGTGACGACTGAATCCGGTGAGAATGGCAAAAGCTCTGCATTAATGAATCGGCCAACGCGCGGGGAGAGGCGGT |
| | | TTGCGTATTGGGCGCTCTTCCGCTTCCTCGCTCACTGACTCGCTGCGCTCGGTCGTTCGGCTGCGGCGAGCGGTATCAGCTCACTCAA |
| | | AGGCGGTAATACGGTTATCCACAGAATCAGGGGATAACGCAGGAAAGAACATGTGAGCAAAAGGCCAGCAAAAGGCCAGGAACCGTA |
| | | AAAAGGCCGCGTTGCTGGCGTTTTTCCATAGGCTCCGCCCCCCTGACGAGCATCACAAAAATCGACGCTCAAGTCAGAGGTGGCGAAA |
| | | CCCGACAGGACTATAAAGATACCAGGCGTTTCCCCCTGGAAGCTCCCTCGTGCGCTCTCCTGTTCCGACCCTGCCGCTTACCGGATAC |
| | | CTGTCCGCCTTTCTCCCTTCGGGAAGCGTGGCGCTTTCTCATAGCTCACGCTGTAGGTATCTCAGTTCGGTGTAGGTCGTTCGCTCCAA |
| | | GCTGGGCTGTGTGCACGAACCCCCCGTTCAGCCCGACCGCTGCGCCTTATCCGGTAACTATCGTCTTGAGTCCAACCCGGTAAGACA |
| | | CGACTTATCGCCACTGGCAGCAGCCACTGGTAACAGGATTAGCAGAGCGAGGTATGTAGGCGGTGCTACAGAGTTCTTGAAGTGGTG |
| | | GCCTAACTACGGCTACACTAGAAGAACAGTATTTGGTATCTGCGCTCTGCTGAAGCCAGTTACCTTCGGAAAAAGAGTTGGTAGCTCTT |
| | | GATCCGGCAAACAAACCACCGCTGGTAGCGGTGGTTTTTTTGTTTGCAAGCAGCAGATTACGCGCAGAAAAAAAGGATCTCAAGAAGAT |
| | | CCTTTGATCTTTTCTACGGGGTCTGACGCTCAGTGGAACGAAAACTCACGTTAAGGGATTTTGGTCATGAGATTATCAAAAAGGATCTTC |
| | | ACCTAGATCCTTTTGATCCGGACAAACAAACAAATACAGTAATTTAGCCAGGACGTCGGCCGAAAGAGCGACAAGCAGATCACGCTTTT |
| | | CGACAGCGTCGGATTTGCGATCGAGGATTTTTCGGCGCTGCGCTACGTCCGCGACCGCGTTGAGGGATCAAGCCACAGCAGCCCACT |
| | | CGACCTTCTAGCCGACCCAGACGAGCCAAGGGATCTTTTTGGAATGCTGCTCCGTCGTCAGGCTTTCCGACGTTTGGGTGGTTGAACA |
| | | GAAGTCATTATCGCACGGAATGCCAAGCACTCCCGAGGGG |
| 315 | DONOR SEQUENCE | ATCTTGTGAATTTTTTTTAGGTTCAGAAGACAGAAGTCATAGCACCACAATTCGATGCAGATGCTAGCTGGTGGACTCAAGGACCCGAT |
| | FOR SOY BIODA | GCTATAATGCAGGTCATCCTCATTTTAAGCTATTAACT |
| | C371D (SEQ ID NO: | |
| | 315) | |
| 316 | DONOR SEQUENCE | TGTTGCTCATTCTTTTGGTGCAGGTTAAAGACAAAACGATTGTTCCCCAGTTCGATGCAGATGCTAGTTGGTGGACACAAGGACCTGAT |
| | FOR RICE BIODA | TCTAACTTACAGGTTATTGTCTACACATCTTCCATCAT |
| | C371D (SEQ ID NO: | |
| | 316) | |
| 316 | DONOR SEQUENCE | GAAGTCTGATTCGTGTACAGGTGAAGGATCATGATTATATTACTCAAATGTTCGATGCTGATGCAAGCTGGTGGACTCAGGGCCCTAAT |
| | FOR SUNFLOWER | GCTGCTTTACAGGTCATTCTGATTTTGTGTATCGTTGA |
| | BIODA C384D (SEQ ID | |
| | NO: 317) | |
| 206 | pBin TMV EcBioA binary | AGCTTGCATGCCTGCAGGTCAACATGGTGGAGCACGACACACTTGTCTACTCCAAAAATATCAAAGATACAGTCTCAGAAGACCAAAGG |
| | vector (SEQ ID NO: 206) | GCAATTGAGACTTTTCAACAAAGGGTAATATCCGGAAACCTCCTCGGATTCCATTGCCCAGCTATCTGTCACTTTATTGTGAAGATAGTG |
| | | GAAAAGGAAGGTGGCTCCTACAAATGCCATCATTGCGATAAAGGAAAGGCCATCGTTGAAGATGCCTCTGCCGACAGTGGTCCCAAAG |
| | | ATGGACCCCCACCCACGAGGAGCATCGTGGAAAAAGAAGACGTTCCAACCACGTCTTCAAAGCAAGTGGATTGATGTGATAACATGGT |
| | | GGAGCACGACACACTTGTCTACTCCAAAAATATCAAAGATACAGTCTCAGAAGACCAAAGGGCAATTGAGACTTTTCAACAAAGGGTAA |
| | | TATCCGGAAACCTCCTCGGATTCCATTGCCCAGCTATCTGTCACTTTATTGTGAAGATAGTGGAAAAGGAAGGTGGCTCTTACAAATGC |
| | | CATCATTGCGATAAAGGAAAGGCCATCGTTGAAGATGCCTCTGCCGACAGTGGTCCCAAAGATGGACCCCCACCCACGAGGAGCATC |
| | | GTGGAAAAAGAAGACGTTCCAACCACGTCTTCAAAGCAAGTGGATTGATGTGATATCTCCACTGACGTAAGGGATGACGCACAATCCCA |
| | | CTATCCTTCGCAAGACCCTTCCTCTATATAAGGAAGTTCATTTCATTTGGAGAGGACCTCGAGTATTTTTACAACAATTACCAACAACAAC |
| | | AAACAACAAACAACATTACAATTACTATTTACAATTACACATATGACTACTGATGATCTTGCTTTTGATCAAAGACATATTTGGCATCCATA |
| | | TACTTCTATGACTTCTCCACTTCCTGTTTATCCTGTTGTTTCTGCTGAAGGATGTGAACTTATTCTTTCTGATGGAAGAAGACTTGTTGAT |
| | | GGAATGTCTTCTTGGTGGGCTGCTATTCATGGATATAATCATCCACAACTTAATGCTGCTATGAAGTCTCAAATTGATGCTATGTCTCAT |
| | | GTTATGTTTGGAGGAATTACTCATGCTCCTGCTATTGAACTTTGTAGAAAGCTTGTTGCTATGACTCCACAACCACTTGAATGTGTTTTTC |
| | | TTGCTGATTCTGGATCTGTTGCTGTTGAAGTTGCTATGAAGATGGCTCTTCAATATTGGCAAGCTAAGGGAGAAGCTAGACAAAGATTTC |
| | | TTACTTTTAGAAATGGATATCATGGAGATACTTTTGGAGCTATGTCTGTTTGTGATCCTGATAATTCTATGCATTCTCTTTGGAAGGGATA |
| | | TCTTCCTGAAAATCTTTTTGCTCCTGCTCCACAATCTAGAATGGATGGAGAATGGGATGAAAGAGATATGGTTGGATTTGCTAGACTTAT |
| | | GGCTGCTCATAGACATGAAATTGCTGCTGTTATTATTGAACCAATTGTTCAAGGAGCTGGAGGAATGAGAATGTATCATCCTGAATGGCT |
| | | TAAGAGAATTAGAAAGATTTGTGATAGAGAAGGAATTCTTCTTATTGCTGATGAAATTGCTACTGGATTTGGAAGAACTGGAAAGCTTTTT |
| | | GCTTGTGAACATGCTGAAATTGCTCCTGATATTCTTTGTCTTGGAAAGGCTCTTACTGGAGGAACTATGACTCTTTCTGCTACTCTTACTA |
| | | CTAGAGAAGTTGCTGAAACTATTTCTAATGGAGAAGCTGGATGTTTTATGCATGGACCAACTTTTATGGGAAATCCACTTGCTTGTGCTG |
| | | CTGCTAATGCTTCTCTTGCTATTCTTGAATCTGGAGATTGGCAACAACAAGTTGCTGATATTGAAGTTCAACTTAGAGAACAACTTGCTC |
| | | CTGCTAGAGATGCTGAAATGGTTGCTGATGTTAGAGTTCTTGGAGCTATTGGAGTTGTTGAAACTACTCATCCTGTTAATATGGCTGCTC |
| | | TTCAAAAGTTTTTTGTTGAACAAGGAGTTTGGATTAGACCATTTGGAAAGCTTATTTATCTTATGCCACCATATATTATTCTTCCACAACAA |
| | | CTTCAAAGACTTACTGCTGCTGTTAATAGAGCTGTTCAAGATGAAACTTTTTTTTGTCAATAAGGTACCGAGCTCGAATTTCCCCGATCG |
| | | TTCAAACATTTGGCAATAAAGTTTCTTAAGATTGAATCCTGTTGCCGGTCTTGCGATGATTATCATATAATTTCTGTTGAATTACGTTAAG |
| | | CATGTAATAATTAACATGTAATGCATGACGTTATTTATGAGATGGGTTTTTATGATTAGAGTCCCGCAATTATACATTTAATACGCGATAG |
| | | AAAACAAAATATAGCGCGCAAACTAGGATAAATTATCGCGCGCGGTGTCATCTATGTTACTAGATCGGGAATTCCTAGAGATCATGAGC |
| | | GGAGAATTAAGGGAGTCACGTTATGACCCCCGCCGATGACGCGGGACAAGCCGTTTTACGTTTGGAACTGACAGAACCGCAACGTTGA |
| | | AGGAGCCACTCAGCCGCGGGTTTCTGGAGTTTAATGAGCTAAGCACATACGTCAGAAACCATTATTGCGCGTTCAAAAGTCGCCTAAG |
| | | GTCACTATCAGCTAGCAAATATTTCTTGTCAAAAATGCTCCACTGACGTTCCATAAATTCCCCTCGGTATCCAATTAGAGTCTCATATTCA |
| | | CTCTCAATCCAAATAATCTGCACCGGATCTGGATCGTTTCGCATGATTGAACAAGATGGATTGCACGCAGGTTCTCCGGCCGCTTGGGT |
| | | GGAGAGGCTATTCGGCTATGACTGGGCACAACAGACAATCGGCTGCTCTGATGCCGCCGTGTTCCGGCTGTCAGCGCAGGGGCGCC |
| | | CGGTTCTTTTTGTCAAGACCGACCTGTCCGGTGCCCTGAATGAACTGCAGGACGAGGCAGCGCGGCTATCGTGGCTGGCCACGACGG |
| | | GCGTTCCTTGCGCAGCTGTGCTCGACGTTGTCACTGAAGCGGGAAGGGACTGGCTGCTATTGGGCGAAGTGCCGGGGCAGGATCTCC |
| | | TGTCATCTCACCTTGCTCCTGCCGAGAAAGTATCCATCATGGCTGATGCAATGCGGCGGCTGCATACGCTTGATCCGGCTACCTGCCC |
| | | ATTCGACCACCAAGCGAAACATCGCATCGAGCGAGCACGTACTCGGATGGAAGCCGGTCTTGTCGATCAGGATGATCTGGACGAAGA |
| | | GCATCAGGGGCTCGCGCCAGCCGAACTGTTCGCCAGGCTCAAGGCGCGCATGCCCGACGGCGAGGATCTCGTCGTGACCCATGGCG |
| | | ATGCCTGCTTGCCGAATATCATGGTGGAAAATGGCCGCTTTTCTGGATTCATCGACTGTGGCCGGCTGGGTGTGGCGGACCGCTATCA |
| | | GGACATAGCGTTGGCTACCCGTGATATTGCTGAAGAGCTTGGCGGCGAATGGGCTGACCGCTTCCTCGTGCTTTACGGTATCGCCGCT |
| | | CCCGATTCGCAGCGCATCGCCTTCTATCGCCTTCTTGACGAGTTCTTCTGAGCGGGACTCTGGGGTTCGAAATGACCGACCAAGCGAC |
| | | GCCCAACCTGCCATCACGAGATTTCGATTCCACCGCCGCCTTCTATGAAAGGTTGGGCTTCGGAATCGTTTTCCGGGACGCCGGCTGG |
| | | ATGATCCTCCAGCGCGGGGATCTCATGCTGGAGTTCTTCGCCCACGGGATCTCTGCGGAACAGGCGGTCGAAGGTGCCGATATCATT |
| | | ACGACAGCAACGGCCGACAAGCACAACGCCACGATCCTGAGCGACAATATGATCGGGCCCGGCGTCCACATCAACGGCGTCGGCGG |
| | | CGACTGCCCAGGCAAGACCGAGATGCACCGCGATATCTTGCTGCGTTCGGATATTTTCGTGGAGTTCCCGCCACAGACCCGGATGATC |
| | | CCCGATCGTTCAAACATTTGGCAATAAAGTTTCTTAAGATTGAATCCTGTTGCCGGTCTTGCGATGATTATCATATAATTTCTGTTGAATT |
| | | ACGTTAAGCATGTAATAATTAACATGTAATGCATGACGTTATTTATGAGATGGGTTTTTATGATTAGAGTCCCGCAATTATACATTTAATAC |
| | | GCGATAGAAAACAAAATATAGCGCGCAAACTAGGATAAATTATCGCGCGCGGTGTCATCTATGTTACTAGATCGGGCCTCCTGTCAATG |
| | | CTGGCGGCGGCTCTGGTGGTGGTTCTGGTGGCGGCTCTGAGGGTGGTGGCTCTGAGGGTGGCGGTTCTGAGGGTGGCGGCTCTGA |
| | | GGGAGGCGGTTCCGGTGGTGGCTCTGGTTCCGGTGATTTTGATTATGAAAAGATGGCAAACGCTAATAAGGGGGCTATGACCGAAAAT |
| | | GCCGATGAAAACGCGCTACAGTCTGACGCTAAAGGCAAACTTGATTCTGTCGCTACTGATTACGGTGCTGCTATCGGCCTGAATGGCG |
| | | CCCGCTCCTTTCGCTTTCTTCCCTTCCTTTCTCGCCACGTTCGCCGGCTTTCCCCGTCAAGCTCTAAATCGGGGGCTCCCTTTAGGGTT |
| | | CCGATTTAGTGCTTTACGGCACCTCGACCCCAAAAAACTTGATTTGGGTGATGGTTCACGTAGTGGGCCATCGCCCTGATAGACGGTTT |
| | | TTCGCCCTTTGACGTTGGAGTCCACGTTCTTTAATAGTGGACTCTTGTTCCAAACTGGAACAACACTCAACCCTATCTCGGGCTATTCTT |
| | | TTGATTTATAAGGGATTTTGCCGATTTCGGAACCACCATCAAACAGGATTTTCGCCTGCTGGGGCAAACCAGCGTGGACCGCTTGCTGC |
| | | AACTCTCTCAGGGCCAGGCGGTGAAGGGCAATCAGCTGTTGCCCGTCTCACTGGTGAAAAGAAAAACCACCCCAGTACATTAAAAACG |
| | | TCCGCAATGTGTTATTAAGTTGTCTAAGCGTCAATTTGTTTACACCACAATATATCCTGCCACCAGCCAGCCAACAGCTCCCCGACCGG |
| | | CAGCTCGGCACAAAATCACCACTCGATACAGGCAGCCCATCAGTCCGGGACGGCGTCAGCGGGAGAGCCGTTGTAAGGCGGCAGAC |
| | | TTTGCTCATGTTACCGATGCTATTCGGAAGAACGGCAACTAAGCTGCCGGGTTTGAAACACGGATGATCTCGCGGAGGGTAGCATGTT |
| | | GATTGTAACGATGACAGAGCGTTGCTGCCTGTGATCAAATATCATCTCCCTCGCAGAGATCCGAATTATCAGCCTTCTTATTCATTTCTC |
| | | GCTTAACCGTGACAGGCTGTCGATCTTGAGAACTATGCCGACATAATAGGAAATCGCTGGATAAAGCCGCTGAGGAAGCTGAGTGGCG |
| | | CTATTTCTTTAGAAGTGAACGTTGACGATATCAACTCCCCTATCCATTGCTCACCGAATGGTACAGGTCGGGGACCCGAAGTTCCGACT |
| | | GTCGGCCTGATGCATCCCCGGCTGATCGACCCCAGATCTGGGGCTGAGAAAGCCCAGTAAGGAAACAACTGTAGGTTCGAGTCGCGA |
| | | GATCCCCCGGAACCAAAGGAAGTAGGTTAAACCCGCTCCGATCAGGCCGAGCCACGCCAGGCCGAGAACATTGGTTCCTGTAGGCAT |
| | | CGGGATTGGCGGATCAAACACTAAAGCTACTGGAACGAGCAGAAGTCCTCCGGCCGCCAGTTGCCAGGCGGTAAAGGTGAGCAGAGG |
| | | CACGGGAGGTTGCCACTTGCGGGTCAGCACGGTTCCGAACGCCATGGAAACCGCCCCCGCCAGGCCCGCTGCGACGCCGACAGGAT |
| | | CTAGCGCTGCGTTTGGTGTCAACACCAACAGCGCCACGCCCGCAGTTCCGCAAATAGCCCCCAGGACCGCCATCAATCGTATCGGGC |
| | | TACCTAGCAGAGCGGCAGAGATGAACACGACCATCAGCGGCTGCACAGCGCCTACCGTCGCCGCGACCCCGCCCGGCAGGCGGTAG |
| | | ACCGAAATAAACAACAAGCTCCAGAATAGCGAAATATTAAGTGCGCCGAGGATGAAGATGCGCATCCACCAGATTCCCGTTGGAATCTG |
| | | TCGGACGATCATCACGAGCAATAAACCCGCCGGCAACGCCCGCAGCAGCATACCGGCGACCCCTCGGCCTCGCTGTTCGGGCTCCAC |
| | | GAAAACGCCGGACAGATGCGCCTTGTGAGCGTCCTTGGGGCCGTCCTCCTGTTTGAAGACCGACAGCCCAATGATCTCGCCGTCGAT |
| | | GTAGGCGCCGAATGCCACGGCATCTCGCAACCGTTCAGCGAACGCCTCCATGGGCTTTTTCTCCTCGTGCTCGTAAACGGACCCGAAC |
| | | ATCTCTGGAGCTTTCTTCAGGGCCGACAATCGGATCTCGCGGAAATCCTGCACGTCGGCCGCTCCAAGCCGTCGAATCTGAGCCTTAA |
| | | TCACAATTGTCAATTTTAATCCTCTGTTTATCGGCAGTTCGTAGAGCGCGCCGTGCGTCCCGAGCGATACTGAGCGAAGCAAGTGCGTC |
| | | GAGCAGTGCCCGCTTGTTCCTGAAATGCCAGTAAAGCGCTGGCTGCTGAACCCCCAGCCGGAACTGACCCCACAAGGCCCTAGCGTT |
| | | TGCAATGCACCAGGTCATCATTGACCCAGGCGTGTTCCACCAGGCCGCTGCCTCGCAACTCTTCGCAGGCTTCGCCGACCTGCTCGC |
| | | GCCACTTCTTCACGCGGGTGGAATCCGATCCGCACATGAGGCGGAAGGTTTCCAGCTTGAGCGGGTACGGCTCCCGGTGCGAGCTGA |
| | | AATAGTCGAACATCCGTCGGGCCGTCGGCGACAGCTTGCGGTACTTCTCCCATATGAATTTCGTGTAGTGGTCGCCAGCAAACAGCAC |
| | | GACGATTTCCTCGTCGATCAGGACCTGGCAACGGGACGTTTTCTTGCCACGGTCCAGGACGCGGAAGCGGTGCAGCAGCGACACCGA |
| | | TTCCAGGTGCCCAACGCGGTCGGACGTGAAGCCCATCGCCGTCGCCTGTAGGCGCGACAGGCATTCCTCGGCCTTCGTGTAATACCG |
| | | GCCATTGATCGACCAGCCCAGGTCCTGGCAAAGCTCGTAGAACGTGAAGGTGATCGGCTCGCCGATAGGGGTGCGCTTCGCGTACTC |
| | | CAACACCTGCTGCCACACCAGTTCGTCATCGTCGGCCCGCAGCTCGACGCCGGTGTAGGTGATCTTCACGTCCTTGTTGACGTGGAAA |
| | | ATGACCTTGTTTTGCAGCGCCTCGCGCGGGATTTTCTTGTTGCGCGTGGTGAACAGGGCAGAGCGGGCCGTGTCGTTTGGCATCGCT |
| | | CGCATCGTGTCCGGCCACGGCGCAATATCGAACAAGGAAAGCTGCATTTCCTTGATCTGCTGCTTCGTGTGTTTCAGCAACGCGGCCT |
| | | GCTTGGCCTCGCTGACCTGTTTTGCCAGGTCCTCGCCGGGGGTTTTTCGCTTCTTGGTCGTCATAGTTCCTCGCGTGTCGATGGTCATC |
| | | GACTTCGCCAAACCTGCCGCCTCCTGTTCGAGACGACGCGAACGCTCCACGGCGGCCGATGGCGCGGGCAGGGCAGGGGGAGCCA |
| | | GTTGCACGCTGTCGCGCTCGATCTTGGCCGTAGCTTGCTGGACCATCGAGCCGACGGACTGGAAGGTTTCGCGGGGCGCACGCATGA |
| | | CGGTGCGGCTTGCGATGGTTTCGGCATCCTCGGCGGAAAACCCCGCGTCGATCAGTTCTTGCCTGTATGCCTTCCGGTCAAACGTCC |
| | | GATTCATTCACCCTCCTTGCGGGATTGCCCCGACTCACGCCGGGGCAATGTGCCCTTATTCCTGATTTGACCCGCCTGGTGCCTTGGT |
| | | GTCCAGATAATCCACCTTATCGGCAATGAAGTCGGTCCCGTAGACCGTCTGGCCGTCCTTCTCGTACTTGGTATTCCGAATCTTGCCCT |
| | | GCACGAATACCAGCGACCCCTTGCCCAAATACTTGCCGTGGGCCTCGGCCTGAGAGCCAAAACACTTGATGCGGAAGAAGTCGGTGC |
| | | GCTCCTGCTTGTCGCCGGCATCGTTGCGCCACATCTAGGTACTAAAACAATTCATCCAGTAAAATATAATATTTTATTTTCTCCCAATCAG |
| | | GCTTGATCCCCAGTAAGTCAAAAAATAGCTCGACATACTGTTCTTCCCCGATATCCTCCCTGATCGACCGGACGCAGAAGGCAATGTCA |
| | | TACCACTTGTCCGCCCTGCCGCTTCTCCCAAGATCAATAAAGCCACTTACTTTGCCATCTTTCACAAAGATGTTGCTGTCTCCCAGGTCG |
| | | CCGTGGGAAAAGACAAGTTCCTCTTCGGGCTTTTCCGTCTTTAAAAAATCATACAGCTCGCGCGGATCTTTAAATGGAGTGTCTTCTTCC |
| | | CAGTTTTCGCAATCCACATCGGCCAGATCGTTATTCAGTAAGTAATCCAATTCGGCTAAGCGGCTGTCTAAGCTATTCGTATAGGGACA |
| | | ATCCGATATGTCGATGGAGTGAAAGAGCCTGATGCACTCCGCATACAGCTCGATAATCTTTTCAGGGCTTTGTTCATCTTCATACTCTTC |
| | | CGAGCAAAGGACGCCATCGGCCTCACTCATGAGCAGATTGCTCCAGCCATCATGCCGTTCAAAGTGCAGGACCTTTGGAACAGGCAG |
| | | CTTTCCTTCCAGCCATAGCATCATGTCCTTTTCCCGTTCCACATCATAGGTGGTCCCTTTATACCGGCTGTCCGTCATTTTTAAATATAG |
| | | GTTTTCATTTTCTCCCACCAGCTTATATACCTTAGCAGGAGACATTCCTTCCGTATCTTTTACGCAGCGGTATTTTTCGATCAGTTTTTTC |
| | | AATTCCGGTGATATTCTCATTTTAGCCATTTATTATTTCCTTCCTCTTTTCTACAGTATTTAAAGATACCCCAAGAAGCTAATTATAACAAG |
| | | ACGAACTCCAATTCACTGTTCCTTGCATTCTAAAACCTTAAATACCAGAAAACAGCTTTTTCAAAGTTGTTTTCAAAGTTGGCGTATAACA |
| | | TAGTATCGACGGAGCCGATTTTGAAACCACAATTATGGGTGATGCTGCCAACTTACTGATTTAGTGTATGATGGTGTTTTTGAGGTGCTC |
| | | CAGTGGCTTCTGTTTCTATCAGCTGTCCCTCCTGTTCAGCTACTGACGGGGTGGTGCGTAACGGCAAAAGCACCGCCGGACATCAGCG |
| | | CTATCTCTGCTCTCACTGCCGTAAAACATGGCAACTGCAGTTCACTTACACCGCTTCTCAACCCGGTACGCACCAGAAAATCATTGATAT |
| | | GGCCATGAATGGCGTTGGATGCCGGGCAACAGCCCGCATTATGGGCGTTGGCCTCAACACGATTTTACGTCACTTAAAAAACTCAGGC |
| | | CGCAGTCGGTAACCTCGCGCATACAGCCGGGCAGTGACGTCATCGTCTGCGCGGAAATGGACGAACAGTGGGGCTATGTCGGGGCT |
| | | AAATCGCGCCAGCGCTGGCTGTTTTACGCGTATGACAGTCTCCGGAAGACGGTTGTTGCGCACGTATTCGGTGAACGCACTATGGCGA |
| | | CGCTGGGGCGTCTTATGAGCCTGCTTCACCCTTTGACGTGGTGATATGGATGACGGATGGCTGGCCGCTGTATGAATCCCGCCTGAAG |
| | | GGAAAGCTGCACGTAATCAGCAAGCGATATACGCAGCGAATTGAGCGGCATAACCTGAATCTGAGGCAGCACCTGGCACGGCTGGGA |
| | | CGGAAGTCGCTGTCGTTCTCAAAATCGGTGGAGCTGCATGACAAAGTCATCGGGCATTATCTGAACATAAAACACTATCAATAAGTTGG |
| | | AGTCATTACCCAATTATGATAGAATTTACAAGCTATAAGGTTATTGTCCTGGGTTTCAAGCATTAGTCCATGCAAGTTTTTATGCTTTGCC |
| | | CATTCTATAGATATATTGATAAGCGCGCTGCCTATGCCTTGCCCCCTGAAATCCTTACATACGGCGATATCTTCTATATAAAAGATATATT |
| | | ATCTTATCAGTATTGTCAATATATTCAAGGCAATCTGCCTCCTCATCCTCTTCATCCTCTTCGTCTTGGTAGCTTTTTAAATATGGCGCTT |
| | | CATAGAGTAATTCTGTAAAGGTCCAATTCTCGTTTTCATACCTCGGTATAATCTTACCTATCACCTCAAATGGTTCGCTGGGTTTATCGCA |
| | | CCCCCGAACACGAGCACGGCACCCGCGACCACTATGCCAAGAATGCCCAAGGTAAAAATTGCCGGCCCCGCCATGAAGTCCGTGAAT |
| | | GCCCCGACGGCCGAAGTGAAGGGCAGGCCGCCACCCAGGCCGCCGCCCTCACTGCCCGGCACCTGGTCGCTGAATGTCGATGCCA |
| | | GCACCTGCGGCACGTCAATGCTTCCGGGCGTCGCGCTCGGGCTGATCGCCCATCCCGTTACTGCCCCGATCCCGGCAATGGCAAGG |
| | | ACTGCCAGCGCTGCCATTTTTGGGGTGAGGCCGTTCGCGGCCGAGGGGCGCAGCCCCTGGGGGGATGGGAGGCCCGCGTTAGCGG |
| | | GCCGGGAGGGTTCGAGAAGGGGGGGCACCCCCCTTCGGCGTGCGCGGTCACGCGCACAGGGCGCAGCCCTGGTTAAAAACAAGGT |
| | | TTATAAATATTGGTTTAAAAGCAGGTTAAAAGACAGGTTAGCGGTGGCCGAAAAACGGGCGGAAACCCTTGCAAATGCTGGATTTTCTG |
| | | CCTGTGGACAGCCCCTCAAATGTCAATAGGTGCGCCCCTCATCTGTCAGCACTCTGCCCCTCAAGTGTCAAGGATCGCGCCCCTCATC |
| | | TGTCAGTAGTCGCGCCCCTCAAGTGTCAATACCGCAGGGCACTTATCCCCAGGCTTGTCCACATCATCTGTGGGAAACTCGCGTAAAA |
| | | TCAGGCGTTTTCGCCGATTTGCGAGGCTGGCCAGCTCCACGTCGCCGGCCGAAATCGAGCCTGCCCCTCATCTGTCAACGCCGCGCC |
| | | GGGTGAGTCGGCCCCTCAAGTGTCAACGTCCGCCCCTCATCTGTCAGTGAGGGCCAAGTTTTCCGCGAGGTATCCACAACGCCGGCG |
| | | GCCGCGGTGTCTCGCACACGGCTTCGACGGCGTTTCTGGCGCGTTTGCAGGGCCATAGACGGCCGCCAGCCCAGCGGCGAGGGCAA |
| | | CCAGCCCGGTGAGCGTCGGAAAGGCGCTCGGTCTTGCCTTGCTCGTCGGTGATGTACTTCACCAGCTCCGCGAAGTCGCTCTTCTTG |
| | | ATGGAGCGCATGGGGACGTGCTTGGCAATCACGCGCACCCCCCGGCCGTTTTAGCGGCTAAAAAAGTCATGGCTCTGCCCTCGGGCG |
| | | GACCACGCCCATCATGACCTTGCCAAGCTCGTCCTGCTTCTCTTCGATCTTCGCCAGCAGGGCGAGGATCGTGGCATCACCGAACCGC |
| | | GCCGTGCGCGGGTCGTCGGTGAGCCAGAGTTTCAGCAGGCCGCCCAGGCGGCCCAGGTCGCCATTGATGCGGGCCAGCTCGCGGA |
| | | CGTGCTCATAGTCCACGACGCCCGTGATTTTGTAGCCCTGGCCGACGGCCAGCAGGTAGGCCGACAGGCTCATGCCGGCCGCCGCC |
| | | GCCTTTTCCTCAATCGCTCTTCGTTCGTCTGGAAGGCAGTACACCTTGATAGGTGGGCTGCCCTTCCTGGTTGGCTTGGTTTCATCAGC |
| | | CATCCGCTTGCCCTCATCTGTTACGCCGGCGGTAGCCGGCCAGCCTCGCAGAGCAGGATTCCCGTTGAGCACCGCCAGGTGCGAATA |
| | | AGGGACAGTGAAGAAGGAACACCCGCTCGCGGGTGGGCCTACTTCACCTATCCTGCCCGGCTGACGCCGTTGGATACACCAAGGAAA |
| | | GTCTACACGAACCCTTTGGCAAAATCCTGTATATCGTGCGAAAAAGGATGGATATACCGAAAAAATCGCTATAATGACCCCGAAGCAGG |
| | | GTTATGCAGCGGAAAAGCGCCACGCTTCCCGAAGGGAGAAAGGCGGACAGGTATCCGGTAAGCGGCAGGGTCGGAACAGGAGAGCG |
| | | CACGAGGGAGCTTCCAGGGGGAAACGCCTGGTATCTTTATAGTCCTGTCGGGTTTCGCCACCTCTGACTTGAGCGTCGATTTTTGTGA |
| | | TGCTCGTCAGGGGGGCGGAGCCTATGGAAAAACGCCAGCAACGCGGCCTTTTTACGGTTCCTGGCCTTTTGCTGGCCTTTTGCTCACA |
| | | TGTTCTTTCCTGCGTTATCCCCTGATTCTGTGGATAACCGTATTACCGCCTTTGAGTGAGCTGATACCGCTCGCCGCAGCCGAACGACC |
| | | GAGCGCAGCGAGTCAGTGAGCGAGGAAGCGGAAGAGCGCCAGAAGGCCGCCAGAGAGGCCGAGCGCGGCCGTGAGGCTTGGACGC |
| | | TAGGGCAGGGCATGAAAAAGCCCGTAGCGGGCTGCTACGGGCGTCTGACGCGGTGGAAAGGGGGAGGGGATGTTGTCTACATGGCT |
| | | CTGCTGTAGTGAGTGGGTTGCGCTCCGGCAGCGGTCCTGATCAATCGTCACCCTTTCTCGGTCCTTCAACGTTCCTGACAACGAGCCT |
| | | CCTTTTCGCCAATCCATCGACAATCACCGCGAGTCCCTGCTCGAACGCTGCGTCCGGACCGGCTTCGTCGAAGGCGTCTATCGCGGC |
| | | CCGCAACAGCGGCGAGAGCGGAGCCTGTTCAACGGTGCCGCCGCGCTCGCCGGCATCGCTGTCGCCGGCCTGCTCCTCAAGCACGG |
| | | CCCCAACAGTGAAGTAGCTGATTGTCATCAGCGCATTGACGGCGTCCCCGGCCGAAAAACCCGCCTCGCAGAGGAAGCGAAGCTGCG |
| | | CGTCGGCCGTTTCCATCTGCGGTGCGCCCGGTCGCGTGCCGGCATGGATGCGCGCGCCATCGCGGTAGGCGAGCAGCGCCTGCCT |
| | | GAAGCTGCGGGCATTCCCGATCAGAAATGAGCGCCAGTCGTCGTCGGCTCTCGGCACCGAATGCGTATGATTCTCCGCCAGCATGGC |
| | | TTCGGCCAGTGCGTCGAGCAGCGCCCGCTTGTTCCTGAAGTGCCAGTAAAGCGCCGGCTGCTGAACCCCCAACCGTTCCGCCAGTTT |
| | | GCGTGTCGTCAGACCGTCTACGCCGACCTCGTTCAACAGGTCCAGGGCGGCACGGATCACTGTATTCGGCTGCAACTTTGTCATGCTT |
| | | GACACTTTATCACTGATAAACATAATATGTCCACCAACTTATCAGTGATAAAGAATCCGCGCGTTCAATCGGACCAGCGGAGGCTGGTC |
| | | CGGAGGCCAGACGTGAAACCCAACATACCCCTGATCGTAATTCTGAGCACTGTCGCGCTCGACGCTGTCGGCATCGGCCTGATTATGC |
| | | CGGTGCTGCCGGGCCTCCTGCGCGATCTGGTTCACTCGAACGACGTCACCGCCCACTATGGCATTCTGCTGGCGCTGTATGCGTTGG |
| | | TGCAATTTGCCTGCGCACCTGTGCTGGGCGCGCTGTCGGATCGTTTCGGGCGGCGGCCAATCTTGCTCGTCTCGCTGGCCGGCGCCA |
| | | GATCTGGGGAACCCTGTGGTTGGCATGCACATACAAATGGACGAACGGATAAACCTTTTCACGCCCTTTTAAATATCCGATTATTCTAAT |
| | | AAACGCTCTTTTCTCTTAGGTTTACCCGCCAATATATCCTGTCAAACACTGATAGTTTAAACTGAAGGCGGGAAACGACAATCTGATCTC |
| | | TAGGA |
| 207 | pCMP EcBioA binary | ATGATTCCCGTGACCGCCACTCTTATTCGTCACAGGTTGAGGCACTTGCGTCATCGAATTAGGTTTAAATCAACCTCTGTATCACCATTC |
| | vector (SEQ ID NO: 207) | CATTTGCCGCTCAATCATCCTACTTATTTGATTTGGTCTGCCAACACTAGCCTGGGCAAAACTTTGGTCAGTACGGGGATAGCAGCCAG |
| | | TTTTCTCTTACAACAACCTAGTTCATCAGCAACAAAACTATTGTATCTCAAACCCATCCAGACAGGTTTTCCTAGTGATTCTGACTCAAGA |
| | | TTTGTTTTTTCAAAGCTCGACTCATTGTCATTGCGAAGACAAATTCCTATAAGTATCTCAAATTCCGTACTTCATTCATCACTCCCTGCTG |
| | | CTAAGTCTCTTGGCCTAAATGTTGAAGTAAGCGAGTCTGGAATGTGTAGTCTCAATTTTCGGGATGAGAAAACTGTTACCGGCGCTCCA |
| | | GAGTTGTTGTGCAAGACACTTTATGCTTGGGAGGCTGCAATTTCACCCCATCTTGCTGCAGAGAGAGAAAACGCAACTGTTGAAGACTC |
| | | TGTGGTTCTTCAAATGATTGAGAAGTGCCTGAAGGAGGAGATGGAGTGCGGAGTTAAATCTGAGAAGAGTGATCTCTTGTGTCTAGTCG |
| | | AAACTGCTGGCGGAGTTGCTTCTCCTGGTCCATCTGGAACCTTGCAATGTGACCTGTATAGGCCGTTTAGGCTCCCCGGAATTCTAGTT |
| | | GGAGACGGCAGGTTGGGGGGTATCTCTGGCACAATAGCAGCCTATGAGAGTCTCAAGCTTCGTGGATACGATATCGCTGCTGTTGTCT |
| | | TCGAAGATCACGGACTAGTTAACGAAGTGCCACTTACATCATATTTGAGGAATAAGGTACCTGTCCTCGTATTACCTCCTGTCCCTAAAG |
| | | ACCCTTCAGACGACTTGATCGAATGGTTCGTGGAGTCCGATGGCGTATTCAAAGCACTTAAGGAGACGATGGTATTAGCGAACCTTGAA |
| | | CGTCTTGAAAGGTTGAATGGTATGGCCAAACTGGCTGGTGAAGTGTTTTGGTGGCCATTCACACAGCATAAGCTTGTTCACCAAGAGAC |
| | | CGTAACTGTGATTGACTCCAGATGTGGCGAGAATTTTTCTATTTATAAGGCATCAGATAATTCTTCTTTGAGTCAACAGTTCGATGCTTGC |
| | | GCAAGTTGGTGGACACAGGGGCCTGACCCTACTTTTCAGGCTGAATTGGCTCGCGAGATGGGATACACAGCGGCCAGGTTCGGCCAT |
| | | GTAATGTTCCCCGAAAATGTTTATGAACCTGCTCTTAAATGTGCTGAGCTTTTGTTAGACGGTGTAGGAAAGGGATGGGCTTCACGCGT |
| | | ATACTTCTCTGATAATGGCTCAACCGCAATAGAGATCGCTTTGAAGATGGCTTTTAGGAAATTTTGCGTTGACCACAACTTTTGCGAAGC |
| | | CACAGAGGAAGAGAAACACATTGTGGTTAAGGTAATTGCCCTGAGAGGCTCATACCACGGCGATACTTTGGGTGCTATGGAGGCTCAG |
| | | GCGCCATCACCATATACTGGTTTTCTTCAGCAACCCTGGTACACGGGCCGGGGCCTATTTTTGGATCCTCCAACCGTTTTTTTGTCTAA |
| | | CGGTTCTTGGAACATATCTTTGCCGGAAAGTTTCTCAGAAATTGCTCCTGAGTACGGCACCTTCACAAGTCGTGATGAAATCTTCGATAA |
| | | GTCAAGAGACGCTTCAACACTTGCAAGAATCTATTCAGCTTATCTCTCAAAGCATTTGCAGGAGCATTCAGGAGTTAGGCAGAGTGCCC |
| | | ATGTTGGTGCATTAATCATTGAGCCGGTGATTCATGGTGCAGGAGGAATGCATATGGTGGATCCTCTATTTCAGAGAGTCCTCGTTAAT |
| | | GAATGTAGAAATCGTAAGATACCTGTCATATTTGACGAAGTCTTCACTGGTTTTTGGAGATTGGGAGTGGAAACGACAACAGAACTTCTA |
| | | GGATGCAAACCTGATATTGCGTGCTTTGCTAAGCTATTGACGGGCGGTATGGTACCCTTGGCTGTGACATTGGCTACAGATGCAGTTTT |
| | | CGATAGTTTTAGTGGTGATAGCAAATTGAAAGCTCTATTGCACGGACATAGTTACTCTGCTCACGCCATGGGATGCGCTACCGCGGCGA |
| | | AGGCAATCCAATGGTTTAAAGATCCTGAAACCAATCACAATATCACATCTCAAGGCAAGACCCTCAGGGAACTGTGGGACGAAGAACTA |
| | | GTTCAGCAAATCTCTTCACACTCAGCAGTGCAACGCGTTGTTGTCATAGGAACCTTGTTTGCTCTCGAGTTGAAGGCAGACGCAAGCAA |
| | | CTCAGGGTACGCTTCACTCTATGCTAAATCTTTATTGATTATGTTGAGGGAAGATGGCATCTTTACACGACCTCTGGGTAACGTGATCTA |
| | | CCTCATGTGCGGTCCTTGTACGAGTCCCGAAATATGTAGACGACTGCTGACTAAACTGTATAAGCGTTTGGGTGAATTTAATCGTACTTA |
| | | AGAGCTCTAGATCCCCGAATTTCCCCGATCGTTCAAACATTTGGCAATAAAGTTTCTTAAGATTGAATCCTGTTGCCGGTCTTGCGATGA |
| | | TTATCATATAATTTCTGTTGAATTACGTTAAGCATGTAATAATTAACATGTAATGCATGACGTTATTTATGAGATGGGTTTTTATGATTAGA |
| | | GTCCCGCAATTATACATTTAATACGCGATAGAAAACAAAATATAGCGCGCAAACTAGGATAAATTATCGCGCGCGGTGTCATCTATGTTA |
| | | CTAGATCGGGAATTGGGTACCAGATCATGAGCGGAGAATTAAGGGAGTCACGTTATGACCCCCGCCGATGACGCGGGACAAGCCGTT |
| | | TTACGTTTGGAACTGACAGAACCGCAACGTTGAAGGAGCCACTCAGCCGCGGGTTTCTGGAGTTTAATGAGCTAAGCACATACGTCAG |
| | | AAACCATTATTGCGCGTTCAAAAGTCGCCTAAGGTCACTATCAGCTAGCAAATATTTCTTGTCAAAAATGCTCCACTGACGTTCCATAAA |
| | | TTCCCCTCGGTATCCAATTAGAGTCTCATATTCACTCTCAATCCAAATAATCTGCACCGGATCTGGATCGTTTCGCATGATTGAACAAGA |
| | | TGGATTGCACGCAGGTTCTCCGGCCGCTTGGGTGGAGAGGCTATTCGGCTATGACTGGGCACAACAGACAATCGGCTGCTCTGATGC |
| | | CGCCGTGTTCCGGCTGTCAGCGCAGGGGCGCCCGGTTCTTTTTGTCAAGACCGACCTGTCCGGTGCCCTGAATGAACTGCAGGACGA |
| | | GGCAGCGCGGCTATCGTGGCTGGCCACGACGGGCGTTCCTTGCGCAGCTGTGCTCGACGTTGTCACTGAAGCGGGAAGGGACTGGC |
| | | TGCTATTGGGCGAAGTGCCGGGGCAGGATCTCCTGTCATCTCACCTTGCTCCTGCCGAGAAAGTATCCATCATGGCTGATGCAATGCG |
| | | GCGGCTGCATACGCTTGATCCGGCTACCTGCCCATTCGACCACCAAGCGAAACATCGCATCGAGCGAGCACGTACTCGGATGGAAGC |
| | | CGGTCTTGTCGATCAGGATGATCTGGACGAAGAGCATCAGGGGCTCGCGCCAGCCGAACTGTTCGCCAGGCTCAAGGCGCGCATGCC |
| | | CGACGGCGAGGATCTCGTCGTGACCCATGGCGATGCCTGCTTGCCGAATATCATGGTGGAAAATGGCCGCTTTTCTGGATTCATCGAC |
| | | TGTGGCCGGCTGGGTGTGGCGGACCGCTATCAGGACATAGCGTTGGCTACCCGTGATATTGCTGAAGAGCTTGGCGGCGAATGGGCT |
| | | GACCGCTTCCTCGTGCTTTACGGTATCGCCGCTCCCGATTCGCAGCGCATCGCCTTCTATCGCCTTCTTGACGAGTTCTTCTGAGCGG |
| | | GACTCTGGGGTTCGAAATGACCGACCAAGCGACGCCCAACCTGCCATCACGAGATTTCGATTCCACCGCCGCCTTCTATGAAAGGTTG |
| | | GGCTTCGGAATCGTTTTCCGGGACGCCGGCTGGATGATCCTCCAGCGCGGGGATCTCATGCTGGAGTTCTTCGCCCACGGGATCTCT |
| | | GCGGAACAGGCGGTCGAAGGTGCCGATATCATTACGACAGCAACGGCCGACAAGCACAACGCCACGATCCTGAGCGACAATATGATC |
| | | GGGCCCGGCGTCCACATCAACGGCGTCGGCGGCGACTGCCCAGGCAAGACCGAGATGCACCGCGATATCTTGCTGCGTTCGGATATT |
| | | TTCGTGGAGTTCCCGCCACAGACCCGGATGATCCCCGATCGTTCAAACATTTGGCAATAAAGTTTCTTAAGATTGAATCCTGTTGCCGG |
| | | TCTTGCGATGATTATCATATAATTTCTGTTGAATTACGTTAAGCATGTAATAATTAACATGTAATGCATGACGTTATTTATGAGATGGGTTT |
| | | TTATGATTAGAGTCCCGCAATTATACATTTAATACGCGATAGAAAACAAAATATAGCGCGCAAACTAGGATAAATTATCGCGCGCGGTGT |
| | | CATCTATGTTACTAGATCGTGCGGCCAGCATGGCCGTATCCGCAATGTGTTATTAAGTTGTCTAAGCGTCAATTTGTTTACACCACAATA |
| | | TATCCTGCCACCAGCCAGCCAACAGCTCCCCGACCGGCAGCTCGGCACAAAATCACCACTCGATACAGGCAGCCCATCAGAATTAAGG |
| | | ATGAATGTCAGCTACTGGGCTATCTGGACAAGGGAAAACGCAAGCGCAAAGAGAAAGCAGGTAGCTTGCAGTGGGCTTACATGGCGAT |
| | | AGCTAGACTGGGCGGTTTTATGGACAGCAAGCGAACCGGAATTGCCAGCTGGGGCGCCCTCTGGTAAGGTTGGGAAGCCCTGCAAAG |
| | | TAAACTGGATGGCTTTCTTGCCGCCAAGGATCTGATGGCGCAGGGGATCAAGATCTGATCAAGAGACAGGATGAGGATCGTTTCGCAT |
| | | GAGCCATATTCAACGGGAAACGTCTTGCTCTAGGCCGCGATTAAATTCCAACATGGATGCTGATTTATATGGGTATAAATGGGCTCGCG |
| | | ATAATGTCGGGCAATCAGGTGCGACAATCTATCGATTGTATGGGAAGCCCGATGCGCCAGAGTTGTTTCTGAAACATGGCAAAGGTAG |
| | | CGTTGCCAATGATGTTACAGATGAGATGGTCAGACTAAACTGGCTGACGGAATTTATGCCTCTTCCGACCATCAAGCATTTTATCCGTAC |
| | | TCCTGATGATGCATGGTTACTCACCACTGCGATCCCCGGCAAAACAGCATTCCAGGTATTAGAAGAATATCCTGATTCAGGTGAAAATA |
| | | TTGTTGATGCGCTGGCAGTGTTCCTGCGCCGGTTGCATTCGATTCCTGTTTGTAATTGTCCTTTTAACAGCGATCGCGTATTTCGTCTCG |
| | | CTCAGGCGCAATCACGAATGAATAACGGTTTGGTTGATGCGAGTGATTTTGATGACGAGCGTAATGGCTGGCCTGTTGAACAAGTCTG |
| | | GAAAGAAATGCATAAACTTTTGCCATTCTCACCGGATTCAGTCGTCACTCATGGTGATTTCTCACTTGATAACCTTATTTTTGACGAGGG |
| | | GAAATTAATAGGTTGTATTGATGTTGGACGAGTCGGAATCGCAGACCGATACCAGGATCTTGCCATCCTATGGAACTGCCTCGGTGAGT |
| | | TTTCTCCTTCATTACAGAAACGGCTTTTTCAAAAATATGGTATTGATAATCCTGATATGAATAAATTGCAGTTTCATTTGATGCTCGATGAG |
| | | TTTTTCTAAGAATTAATTCATCCCGGGGATCTGGCTCGCGGCGGACGCACGACGCCGGGGCGAGACCATAGGCGATCTCCTAAATCAA |
| | | TAGTAGCTGTAACCTCGAAGCGTTTCACTTGTAACAACGATTGAGAATTTTTGTCATAAAATTGAAATACTTGGTTCGCATTTTTGTCATC |
| | | CGCGGTCAGCCGCAATTCTGACGAACTGCCCATTTAGCTGGAGATGATTGTACATCCTTCACGTGAAAATTTCTCAAGCGCTGTGAACA |
| | | AGGGTTCAGATTTTAGATTGAAAGGTGAGCCGTTGAAACACGTTCTTCTTGTCGATGACGACGTCGCTATGCGGCATCTTATTATTGAAT |
| | | ACCTTACGATCCACGCCTTCAAAGTGACCGCGGTAGCCGACAGCACCCAGTTCACAAGAGTACTCTCTTCCGCGACGGTCGATGTCGT |
| | | GGTTGTTGATCTAGATTTAGGTCGTGAAGATGGGCTCGAGATCGTTCGTAATCTGGCGGCAAAGTCTGATATTCCAATCATAATTATCAG |
| | | TGGCGACCGCCTTGAGGAGACGGATAAAGTTGTTGCACTCGAGCTAGGAGCAAGTGATTTTATCGCTAAGCCGTTCAGTATCAGAGAG |
| | | TTTCTAGCACGCATTCGGGTTGCCTTGCGCGTGCGCCCCAACGTTGTCCGCTCCAAAGACCGACGGTCTTTTTGTTTTACTGACTGGAC |
| | | ACTTAATCTCAGGCAACGTCGCTTGATGTCCGAAGCTGGCGGTGAGGTGAAACTTACGGCAGGTGAGTTCAATCTTCTCCTCGCGTTTT |
| | | TAGAGAAACCCCGCGACGTTCTATCGCGCGAGCAACTTCTCATTGCCAGTCGAGTACGCGACGAGGAGGTTTATGACAGGAGTATAGA |
| | | TGTTCTCATTTTGAGGCTGCGCCGCAAACTTGAGGCAGATCCGTCAAGCCCTCAACTGATAAAAACAGCAAGAGGTGCCGGTTATTTCT |
| | | TTGACGCGGACGTGCAGGTTTCGCACGGGGGGACGATGGCAGCCTGAGCCAATTCCCAGATCCCCGAGGAATCGGCGTGAGCGGTC |
| | | GCAAACCATCCGGCCCGGTACAAATCGGCGCGGCGCTGGGTGATGACCTGGTGGAGAAGTTGAAGGCCGCGCAGGCCGCCCAGCG |
| | | GCAACGCATCGAGGCAGAAGCACGCCCCGGTGAATCGTGGCAAGCGGCCGCTGATCGAATCCGCAAAGAATCCCGGCAACCGCCGG |
| | | CAGCCGGTGCGCCGTCGATTAGGAAGCCGCCCAAGGGCGACGAGCAACCAGATTTTTTCGTTCCGATGCTCTATGACGTGGGCACCC |
| | | GCGATAGTCGCAGCATCATGGACGTGGCCGTTTTCCGTCTGTCGAAGCGTGACCGACGAGCTGGCGAGGTGATCCGCTACGAGCTTC |
| | | CAGACGGGCACGTAGAGGTTTCCGCAGGGCCGGCCGGCATGGCCAGTGTGTGGGATTACGACCTGGTACTGATGGCGGTTTCCCATC |
| | | TAACCGAATCCATGAACCGATACCGGGAAGGGAAGGGAGACAAGCCCGGCCGCGTGTTCCGTCCACACGTTGCGGACGTACTCAAGT |
| | | TCTGCCGGCGAGCCGATGGCGGAAAGCAGAAAGACGACCTGGTAGAAACCTGCATTCGGTTAAACACCACGCACGTTGCCATGCAGC |
| | | GTACGAAGAAGGCCAAGAACGGCCGCCTGGTGACGGTATCCGAGGGTGAAGCCTTGATTAGCCGCTACAAGATCGTAAAGAGCGAAA |
| | | CCGGGCGGCCGGAGTACATCGAGATCGAGCTAGCTGATTGGATGTACCGCGAGATCACAGAAGGCAAGAACCCGGACGTGCTGACG |
| | | GTTCACCCCGATTACTTTTTGATCGATCCCGGCATCGGCCGTTTTCTCTACCGCCTGGCACGCCGCGCCGCAGGCAAGGCAGAAGCCA |
| | | GATGGTTGTTCAAGACGATCTACGAACGCAGTGGCAGCGCCGGAGAGTTCAAGAAGTTCTGTTTCACCGTGCGCAAGCTGATCGGGTC |
| | | AAATGACCTGCCGGAGTACGATTTGAAGGAGGAGGCGGGGCAGGCTGGCCCGATCCTAGTCATGCGCTACCGCAACCTGATCGAGG |
| | | GCGAAGCATCCGCCGGTTCCTAATGTACGGAGCAGATGCTAGGGCAAATTGCCCTAGCAGGGGAAAAAGGTCGAAAAGGTCTCTTTCC |
| | | TGTGGATAGCACGTACATTGGGAACCCAAAGCCGTACATTGGGAACCGGAACCCGTACATTGGGAACCCAAAGCCGTACATTGGGAAC |
| | | CGGTCACACATGTAAGTGACTGATATAAAAGAGAAAAAAGGCGATTTTTCCGCCTAAAACTCTTTAAAACTTATTAAAACTCTTAAAACCC |
| | | GCCTGGCCTGTGCATAACTGTCTGGCCAGCGCACAGCCGAAGAGCTGCAAAAAGCGCCTACCCTTCGGTCGCTGCGCTCCCTACGCC |
| | | CCGCCGCTTCGCGTCGGCCTATCGCGGCCGCTGGCCGCTCAAAAATGGCTGGCCTACGGCCAGGCAATCTACCAGGGCGCGGACAA |
| | | GCCGCGCCGTCGCCACTCGACCGCCGGCGCTGAGGTCTGCCTCGTGAAGAAGGTGTTGCTGACTCATACCAGGCCTGAATCGCCCCA |
| | | TCATCCAGCCAGAAAGTGAGGGAGCCACGGTTGATGAGAGCTTTGTTGTAGGTGGACCAGTTGGTGATTTTGAACTTTTGCTTTGCCAC |
| | | GGAACGGTCTGCGTTGTCGGGAAGATGCGTGATCTGATCCTTCAACTCAGCAAAAGTTCGATTTATTCAACAAAGCCGCCGTCCCGTCA |
| | | AGTCAGCGTAATGCTCTGCCAGTGTTACAACCAATTAACCAATTCTGATTAGAAAAACTCATCGAGCATCAAATGAAACTGCAATTTATTC |
| | | ATATCAGGATTATCAATACCATATTTTTGAAAAAGCCGTTTCTGTAATGAAGGAGAAAACTCACCGAGGCAGTTCCATAGGATGGCAAGA |
| | | TCCTGGTATCGGTCTGCGATTCCGACTCGTCCAACATCAATACAACCTATTAATTTCCCCTCGTCAAAAATAAGGTTATCAAGTGAGAAA |
| | | TCACCATGAGTGACGACTGAATCCGGTGAGAATGGCAAAAGCTCTGCATTAATGAATCGGCCAACGCGCGGGGAGAGGCGGTTTGCG |
| | | TATTGGGCGCTCTTCCGCTTCCTCGCTCACTGACTCGCTGCGCTCGGTCGTTCGGCTGCGGCGAGCGGTATCAGCTCACTCAAAGGC |
| | | GGTAATACGGTTATCCACAGAATCAGGGGATAACGCAGGAAAGAACATGTGAGCAAAAGGCCAGCAAAAGGCCAGGAACCGTAAAAAG |
| | | GCCGCGTTGCTGGCGTTTTTCCATAGGCTCCGCCCCCCTGACGAGCATCACAAAAATCGACGCTCAAGTCAGAGGTGGCGAAACCCG |
| | | ACAGGACTATAAAGATACCAGGCGTTTCCCCCTGGAAGCTCCCTCGTGCGCTCTCCTGTTCCGACCCTGCCGCTTACCGGATACCTGT |
| | | CCGCCTTTCTCCCTTCGGGAAGCGTGGCGCTTTCTCATAGCTCACGCTGTAGGTATCTCAGTTCGGTGTAGGTCGTTCGCTCCAAGCT |
| | | GGGCTGTGTGCACGAACCCCCCGTTCAGCCCGACCGCTGCGCCTTATCCGGTAACTATCGTCTTGAGTCCAACCCGGTAAGACACGA |
| | | CTTATCGCCACTGGCAGCAGCCACTGGTAACAGGATTAGCAGAGCGAGGTATGTAGGCGGTGCTACAGAGTTCTTGAAGTGGTGGCCT |
| | | AACTACGGCTACACTAGAAGAACAGTATTTGGTATCTGCGCTCTGCTGAAGCCAGTTACCTTCGGAAAAAGAGTTGGTAGCTCTTGATC |
| | | CGGCAAACAAACCACCGCTGGTAGCGGTGGTTTTTTTGTTTGCAAGCAGCAGATTACGCGCAGAAAAAAAGGATCTCAAGAAGATCCTT |
| | | TGATCTTTTCTACGGGGTCTGACGCTCAGTGGAACGAAAACTCACGTTAAGGGATTTTGGTCATGAGATTATCAAAAAGGATCTTCACCT |
| | | AGATCCTTTTGATCCGGAATTAATTCCTGTGGTTGGCATGCACATACAAATGGACGAACGGATAAACCTTTTCACGCCCTTTTAAATATC |
| | | CGATTATTCTAATAAACGCTCTTTTCTCTTAGGTTTACCCGCCAATATATCCTGTCAAACACTGATAGTTTAAACTGAAGGCGGGAAACG |
| | | ACAATCTGATCATGAGCGGAGAATTAAGGGAGTCACGTTATGACCCCCGCCGATGACGCGGGACAAGCCGTTTTACGTTTGGAACTGA |
| | | CAGAACCGCAACGCTGCAGGAATTGGCCGCAGCGGCCATTTAAATCAATTGGGCGCGTACGTAGCACTAGTGCGCGATCGCTTAATTA |
| | | AGCGGCGCGCCTAAAGCTTCTGGCAGACAAAGTGGCAGACATACTGTCCCACAAATGAAGATGGAATCTGTAAAAGAAAACGCGTGAA |
| | | ATAATGCGTCTGACAAAGGTTAGGTCGGCTGCCTTTAATCAATACCAAAGTGGTCCCTACCACGATGGAAAAACTGTGCAGTCGGTTTG |
| | | GCTTTTTCTGACGAACAAATAAGATTCGTGGCCGACAGGTGGGGGTCCACCATGTGAAGGCATCTTCAGACTCCAATAATGGAGCAAT |
| | | GACGTAAGGGCTTACGAAATAAGTAAGGGTAGTTTGGGAAATGTCCACTCACCCGTCAGTCTATAAATACTTAGCCCCTCCCTCATTGT |
| | | TAAGGGAGCAAGGATCCTAAACC |
| 311 | pET-24 His-AtBIO3-BIO1 | GGCAGCAGCCATCATCATCATCATCACAGCAGCGGCCTGGTGCCGCGCGGCAGCCATATGAAGTCCACCAGCGTGTCCCCGTTTCAC |
| | E. coli expression vector | TTACCTCTGAATCACCCTACGTACCTGATTTGGAGTGCTAACACAAGCCTCGGTAAGACTCTCGTCTCTACCGGCATCGCGGCAAGCTT |
| | (SEQ ID NO: 311) | CCTTTTGCAGCAGCCGTCAAGTAGTGCGACTAAACTGCTCTACCTCAAGCCAATTCAGACTGGCTTCCCTTCAGACTCAGACTCCCGCT |
| | | TCGTGTTTTCAAAGTTAGACTCATTATCCCTGCGTCGCCAAATCCCAATTTCTATCTCAAACAGCGTGTTGCATTCATCACTGCCAGCCG |
| | | CTAAATCCCTTGGGCTGAACGTAGAAGTTAGTGAGTCTGGCATGTGCTCCCTTAACTTTCGCGACGAAAAGACAGTTACGGGCGCGCC |
| | | TGAGCTGCTGTGTAAGACGCTTTACGCATGGGAAGCAGCGATCTCCCCGCATTTAGCGGCCGAGCGCGAAAATGCTACAGTCGAGGA |
| | | CTCTGTTGTCCTGCAAATGATCGAGAAGTGCCTCAAAGAGGAGATGGAGTGTGGAGTTAAGAGCGAGAAGAGTGATCTGTTGTGTCTT |
| | | GTAGAAACTGCTGGCGGCGTGGCGAGCCCTGGCCCGTCCGGTACGTTACAATGTGACTTATACCGTCCCTTTCGTCTCCCAGGAATCT |
| | | TGGTCGGGGACGGCCGTCTGGGCGGTATCAGCGGAACAATTGCGGCATACGAATCGCTGAAGCTTCGTGGTTACGATATCGCGGCAG |
| | | TCGTGTTTGAAGATCACGGCCTCGTGAACGAGGTGCCACTGACTTCATACTTACGTAACAAGGTCCCAGTGTTAGTCTTACCGCCTGTC |
| | | CCAAAGGACCCGTCGGACGATCTCATCGAGTGGTTTGTCGAGTCAGACGGTGTATTTAAGGCGCTGAAGGAGACGATGGTATTAGCTA |
| | | ACTTAGAGCGTCTGGAGCGTCTTAACGGTATGGCGAAGCTGGCGGGTGAGGTGTTTTGGTGGCCGTTTACACAACATAAGTTAGTCCA |
| | | TCAAGAGACCGTAACCGTAATTGACAGTCGTTGCGGCGAGAACTTTTCGATCTATAAGGCCAGCGATAATAGTTCCTTATCTCAACAGTT |
| | | TGATGCGTGTGCATCGTGGTGGACACAGGGGCCCGACCCTACTTTTCAAGCGGAACTGGCACGTGAAATGGGCTATACAGCTGCTCG |
| | | TTTTGGTCATGTCATGTTTCCCGAAAATGTATACGAGCCAGCGTTAAAGTGCGCCGAGTTGCTCTTGGACGGAGTAGGGAAGGGTTGG |
| | | GCATCGCGCGTATACTTTTCTGACAATGGCAGTACGGCTATCGAAATCGCCTTAAAGATGGCATTCCGTAAGTTCTGTGTCGATCACAA |
| | | CTTTTGCGAGGCAACGGAAGAGGAAAAGCACATTGTCGTGAAGGTGATCGCCCTTCGCGGGTCCTACCATGGGGATACCTTGGGCGC |
| | | GATGGAAGCGCAGGCTCCGAGTCCTTATACCGGTTTCCTTCAACAACCTTGGTATACTGGCCGTGGGTTATTCTTGGACCCGCCGACG |
| | | GTGTTTCTCTCTAATGGTAGTTGGAATATTTCCTTACCAGAGTCGTTTAGCGAGATTGCACCTGAATACGGGACGTTCACTAGTCGTGAC |
| | | GAAATCTTTGACAAGAGCCGTGACGCGAGCACGTTAGCCCGCATCTACAGCGCGTATCTGAGCAAGCACTTGCAGGAACACAGTGGTG |
| | | TGCGCCAAAGTGCGCACGTCGGTGCCTTGATTATCGAGCCCGTCATCCACGGGGGGGGCGGAATGCACATGGTGGACCCGTTATTCC |
| | | AGCGTGTCCTCGTGAATGAATGCCGTAATCGTAAGATCCCGGTCATCTTTGACGAGGTATTTACAGGGTTTTGGCGTCTTGGGGTTGAG |
| | | ACGACGACCGAGTTGCTTGGCTGTAAGCCGGATATTGCGTGTTTTGCCAAACTCTTAACTGGTGGAATGGTACCTTTAGCAGTTACGCT |
| | | GGCGACAGACGCAGTGTTCGACTCTTTCTCCGGCGATAGCAAGTTAAAGGCACTCCTTCACGGACATAGCTACAGCGCGCACGCAATG |
| | | GGCTGCGCCACCGCCGCCAAGGCCATCCAATGGTTCAAGGACCCGGAGACTAATCATAACATTACCTCGCAGGGTAAAACTTTACGCG |
| | | AGTTATGGGACGAGGAACTCGTTCAGCAAATTAGTAGCCACAGTGCGGTCCAACGCGTTGTGGTTATCGGAACGTTATTTGCGCTCGA |
| | | GCTTAAGGCAGACGCGTCTAACAGTGGCTACGCCTCTCTTTATGCGAAAAGCCTGCTGATTATGTTGCGCGAGGACGGTATCTTTACCC |
| | | GCCCCTTAGGTAATGTCATCTACCTTATGTGCGGACCTTGTACCAGCCCAGAGATCTGCCGCCGCCTCTTGACCAAGCTTTATAAGCGT |
| | | CTGGGTGAGTTTAACCGCACCTAAGCGGCCGCACTCGAGCACCACCACCACCACCACTGAGATCCGGCTGCTAACAAAGCCCGAAAG |
| | | GAAGCTGAGTTGGCTGCTGCCACCGCTGAGCAATAACTAGCATAACCCCTTGGGGCCTCTAAACGGGTCTTGAGGGGTTTTTTGCTGA |
| | | AAGGAGGAACTATATCCGGATTGGCGAATGGGACGCGCCCTGTAGCGGCGCATTAAGCGCGGCGGGTGTGGTGGTTACGCGCAGCG |
| | | TGACCGCTACACTTGCCAGCGCCCTAGCGCCCGCTCCTTTCGCTTTCTTCCCTTCCTTTCTCGCCACGTTCGCCGGCTTTCCCCGTCAA |
| | | GCTCTAAATCGGGGGCTCCCTTTAGGGTTCCGATTTAGTGCTTTACGGCACCTCGACCCCAAAAAACTTGATTAGGGTGATGGTTCACG |
| | | TAGTGGGCCATCGCCCTGATAGACGGTTTTTCGCCCTTTGACGTTGGAGTCCACGTTCTTTAATAGTGGACTCTTGTTCCAAACTGGAA |
| | | CAACACTCAACCCTATCTCGGTCTATTCTTTTGATTTATAAGGGATTTTGCCGATTTCGGCCTATTGGTTAAAAAATGAGCTGATTTAACA |
| | | AAAATTTAACGCGAATTTTAACAAAATATTAACGTTTACAATTTCAGGTGGCACTTTTCGGGGAAATGTGCGCGGAACCCCTATTTGTTTA |
| | | TTTTTCTAAATACATTCAAATATGTATCCGCTCATGAATTAATTCTTAGAAAAACTCATCGAGCATCAAATGAAACTGCAATTTATTCATAT |
| | | CAGGATTATCAATACCATATTTTTGAAAAAGCCGTTTCTGTAATGAAGGAGAAAACTCACCGAGGCAGTTCCATAGGATGGCAAGATCCT |
| | | GGTATCGGTCTGCGATTCCGACTCGTCCAACATCAATACAACCTATTAATTTCCCCTCGTCAAAAATAAGGTTATCAAGTGAGAAATCAC |
| | | CATGAGTGACGACTGAATCCGGTGAGAATGGCAAAAGTTTATGCATTTCTTTCCAGACTTGTTCAACAGGCCAGCCATTACGCTCGTCA |
| | | TCAAAATCACTCGCATCAACCAAACCGTTATTCATTCGTGATTGCGCCTGAGCGAGACGAAATACGCGATCGCTGTTAAAAGGACAATT |
| | | ACAAACAGGAATCGAATGCAACCGGCGCAGGAACACTGCCAGCGCATCAACAATATTTTCACCTGAATCAGGATATTCTTCTAATACCT |
| | | GGAATGCTGTTTTCCCGGGGATCGCAGTGGTGAGTAACCATGCATCATCAGGAGTACGGATAAAATGCTTGATGGTCGGAAGAGGCAT |
| | | AAATTCCGTCAGCCAGTTTAGTCTGACCATCTCATCTGTAACATCATTGGCAACGCTACCTTTGCCATGTTTCAGAAACAACTCTGGCGC |
| | | ATCGGGCTTCCCATACAATCGATAGATTGTCGCACCTGATTGCCCGACATTATCGCGAGCCCATTTATACCCATATAAATCAGCATCCAT |
| | | GTTGGAATTTAATCGCGGCCTAGAGCAAGACGTTTCCCGTTGAATATGGCTCATAACACCCCTTGTATTACTGTTTATGTAAGCAGACAG |
| | | TTTTATTGTTCATGACCAAAATCCCTTAACGTGAGTTTTCGTTCCACTGAGCGTCAGACCCCGTAGAAAAGATCAAAGGATCTTCTTGAG |
| | | ATCCTTTTTTTCTGCGCGTAATCTGCTGCTTGCAAACAAAAAAACCACCGCTACCAGCGGTGGTTTGTTTGCCGGATCAAGAGCTACCA |
| | | ACTCTTTTTCCGAAGGTAACTGGCTTCAGCAGAGCGCAGATACCAAATACTGTCCTTCTAGTGTAGCCGTAGTTAGGCCACCACTTCAA |
| | | GAACTCTGTAGCACCGCCTACATACCTCGCTCTGCTAATCCTGTTACCAGTGGCTGCTGCCAGTGGCGATAAGTCGTGTCTTACCGGG |
| | | TTGGACTCAAGACGATAGTTACCGGATAAGGCGCAGCGGTCGGGCTGAACGGGGGGTTCGTGCACACAGCCCAGCTTGGAGCGAAC |
| | | GACCTACACCGAACTGAGATACCTACAGCGTGAGCTATGAGAAAGCGCCACGCTTCCCGAAGGGAGAAAGGCGGACAGGTATCCGGT |
| | | AAGCGGCAGGGTCGGAACAGGAGAGCGCACGAGGGAGCTTCCAGGGGGAAACGCCTGGTATCTTTATAGTCCTGTCGGGTTTCGCCA |
| | | CCTCTGACTTGAGCGTCGATTTTTGTGATGCTCGTCAGGGGGGCGGAGCCTATGGAAAAACGCCAGCAACGCGGCCTTTTTACGGTTC |
| | | CTGGCCTTTTGCTGGCCTTTTGCTCACATGTTCTTTCCTGCGTTATCCCCTGATTCTGTGGATAACCGTATTACCGCCTTTGAGTGAGCT |
| | | GATACCGCTCGCCGCAGCCGAACGACCGAGCGCAGCGAGTCAGTGAGCGAGGAAGCGGAAGAGCGCCTGATGCGGTATTTTCTCCTT |
| | | ACGCATCTGTGCGGTATTTCACACCGCATATATGGTGCACTCTCAGTACAATCTGCTCTGATGCCGCATAGTTAAGCCAGTATACACTC |
| | | CGCTATCGCTACGTGACTGGGTCATGGCTGCGCCCCGACACCCGCCAACACCCGCTGACGCGCCCTGACGGGCTTGTCTGCTCCCG |
| | | GCATCCGCTTACAGACAAGCTGTGACCGTCTCCGGGAGCTGCATGTGTCAGAGGTTTTCACCGTCATCACCGAAACGCGCGAGGCAG |
| | | CTGCGGTAAAGCTCATCAGCGTGGTCGTGAAGCGATTCACAGATGTCTGCCTGTTCATCCGCGTCCAGCTCGTTGAGTTTCTCCAGAA |
| | | GCGTTAATGTCTGGCTTCTGATAAAGCGGGCCATGTTAAGGGCGGTTTTTTCCTGTTTGGTCACTGATGCCTCCGTGTAAGGGGGATTT |
| | | CTGTTCATGGGGGTAATGATACCGATGAAACGAGAGAGGATGCTCACGATACGGGTTACTGATGATGAACATGCCCGGTTACTGGAAC |
| | | GTTGTGAGGGTAAACAACTGGCGGTATGGATGCGGCGGGACCAGAGAAAAATCACTCAGGGTCAATGCCAGCGCTTCGTTAATACAGA |
| | | TGTAGGTGTTCCACAGGGTAGCCAGCAGCATCCTGCGATGCAGATCCGGAACATAATGGTGCAGGGCGCTGACTTCCGCGTTTCCAG |
| | | ACTTTACGAAACACGGAAACCGAAGACCATTCATGTTGTTGCTCAGGTCGCAGACGTTTTGCAGCAGCAGTCGCTTCACGTTCGCTCGC |
| | | GTATCGGTGATTCATTCTGCTAACCAGTAAGGCAACCCCGCCAGCCTAGCCGGGTCCTCAACGACAGGAGCACGATCATGCGCACCC |
| | | GTGGGGCCGCCATGCCGGCGATAATGGCCTGCTTCTCGCCGAAACGTTTGGTGGGGGGACCAGTGACGAAGGCTTGAGCGAGGGCG |
| | | TGCAAGATTCCGAATACCGCAAGCGACAGGCCGATCATCGTCGCGCTCCAGCGAAAGCGGTCCTCGCCGAAAATGACCCAGAGCGCT |
| | | GCCGGCACCTGTCCTACGAGTTGCATGATAAAGAAGACAGTCATAAGTGCGGCGACGATAGTCATGCCCCGCGCCCACCGGAAGGAG |
| | | CTGACTGGGTTGAAGGCTCTCAAGGGCATCGGTCGAGATCCCGGTGCCTAATGAGTGAGCTAACTTACATTAATTGCGTTGCGCTCAC |
| | | TGCCCGCTTTCCAGTCGGGAAACCTGTCGTGCCAGCTGCATTAATGAATCGGCCAACGCGCGGGGAGAGGCGGTTTGCGTATTGGGC |
| | | GCCAGGGTGGTTTTTCTTTTCACCAGTGAGACGGGCAACAGCTGATTGCCCTTCACCGCCTGGCCCTGAGAGAGTTGCAGCAAGCGGT |
| | | CCACGCTGGTTTGCCCCAGCAGGCGAAAATCCTGTTTGATGGTGGTTAACGGGGGGATATAACATGAGCTGTCTTCGGTATCGTCGTA |
| | | TCCCACTACCGAGATATCCGCACCAACGCGCAGCCCGGACTCGGTAATGGCGCGCATTGCGCCCAGCGCCATCTGATCGTTGGCAAC |
| | | CAGCATCGCAGTGGGAACGATGCCCTCATTCAGCATTTGCATGGTTTGTTGAAAACCGGACATGGCACTCCAGTCGCCTTCCCGTTCC |
| | | GCTATCGGCTGAATTTGATTGCGAGTGAGATATTTATGCCAGCCAGCCAGACGCAGACGCGCCGAGACAGAACTTAATGGGCCCGCTA |
| | | ACAGCGCGATTTGCTGGTGACCCAATGCGACCAGATGCTCCACGCCCAGTCGCGTACCGTCTTCATGGGAGAAAATAATACTGTTGAT |
| | | GGGTGTCTGGTCAGAGACATCAAGAAATAACGCCGGAACATTAGTGCAGGCAGCTTCCACAGCAATGGCATCCTGGTCATCCAGCGGA |
| | | TAGTTAATGATCAGCCCACTGACGCGTTGCGCGAGAAGATTGTGCACCGCCGCTTTACAGGCTTCGACGCCGCTTCGTTCTACCATCG |
| | | ACACCACCACGCTGGCACCCAGTTGATCGGCGCGAGATTTAATCGCCGCGACAATTTGCGACGGCGCGTGCAGGGCCAGACTGGAG |
| | | GTGGCAACGCCAATCAGCAACGACTGTTTGCCCGCCAGTTGTTGTGCCACGCGGTTGGGAATGTAATTCAGCTCCGCCATCGCCGCTT |
| | | CCACTTTTTCCCGCGTTTTCGCAGAAACGTGGCTGGCCTGGTTCACCACGCGGGAAACGGTCTGATAAGAGACACCGGCATACTCTGC |
| | | GACATCGTATAACGTTACTGGTTTCACATTCACCACCCTGAATTGACTCTCTTCCGGGCGCTATCATGCCATACCGCGAAAGGTTTTGC |
| | | GCCATTCGATGGTGTCCGGGATCTCGACGCTCTCCCTTATGCGACTCCTGCATTAGGAAGCAGCCCAGTAGTAGGTTGAGGCCGTTGA |
| | | GCACCGCCGCCGCAAGGAATGGTGCATGCAAGGAGATGGCGCCCAACAGTCCCCCGGCCACGGGGCCTGCCACCATACCCACGCCG |
| | | AAACAAGCGCTCATGAGCCCGAAGTGGCGAGCCCGATCTTCCCCATCGGTGATGTCGGCGATATAGGCGCCAGCAACCGCACCTGTG |
| | | GCGCCGGTGATGCCGGCCACGATGCGTCCGGCGTAGAGGATCGAGATCTCGATCCCGCGAAATTAATACGACTCACTATAGGGGAAT |
| | | TGTGAGCGGATAACAATTCCCCTCTAGAAATAATTTTGTTTAACTTTAAGAAGGAGATATACATATG |
| | MOTIFS | |
| 208 | | W;(H/Y/W);P;F;(A/Q/S/T);Q;(H/Q/V);X;X;X [Motif 1 (SEQ ID NO: 208)] |
| 209 | | (I/L/V);(D/E);(S/G);(R/A);X;(A/D/G/K);(E/D/N);X;(F/Y) [Motif 2 (SEQ ID NO: 209)] |
| 210 | | (F/I/L/V/Y);D;(A/G);(C/I/P/S);(A/G/S);S;W;W;(T/S/V);(I/Q) [Motif 3 (SEQ ID NO: 210)] |
| 2011 | | (F/Y);(G/D);(H/Q);(A/I/V);(M/I/L);(F/L/Y);(A/L/P);(E/G/H/L/N/S/T) [Motif 4 (SEQ ID NO: 211)] |
| 212 | | (A/G/T);(F/L/T/Q/V);X;(G/N/D/E/R);(S/C/G);Y;H;G;D;T;(L/I/M);(G/S);(A/C/S/T/V); |
| | | (M/L/T);(D/E/N);X;(A/E/K/Q/R/S/T);(A/E/I/Q/V/T);(E/G/I/K/P/S);(C/E/N/S/T);X;(F/T/Y); |
| | | (M/N/S/T);X [Motif 5 (SEQ ID NO: 212)] |
| 213 | | (A/E/K/Q/R/S/T);(E/H/I/Q/V/T);(D/E/H/P);(S/W);(F/H/Y);X;(G/P/Q/R/S);(E/K/Q/R/W) |
| | | [Motif 6 (SEQ ID NO: 213)] |
| 214 | | (F/S/W);(F/I/L/P);X;(A/C/F/P/V/Y);(P/S);X;(I/L/M/T/V) [Motif 7 (SEQ ID NO: 214)] |
| 215 | | (A/G);(A/S);(A/G);G;M;X;(F/M/L);(A/C/I/V) [Motif 8 (SEQ ID NO: 215)] |
| 216 | | L;L;H;(G/R);H;S;(F/Y);(N/S/T);(A/G/T);(H/N/Y) [Motif 9 (SEQ ID NO: 216)] |
| 217 | | (A/E/G/H/K/P/Q/S/V);A/C/G/K/N/Q/R/S/T/V/Y);(H/I/L/V);(A/D/E/H/K/L/N/Q/R/S);(G/K/R/S/Y); |
| | | (A/C/I/L/T/V);(F/I/M/T/V/W);(A/D/G/N/S/V;(I/L/M/Q/V) [Motif 10 (SEQ ID NO: 217)] |
| 218 | | (A/I/L/V/Y);(A/I/L/N/Q/R/V);(A/D/E/I/K/L/M/N/T/Q/R/S);(A/D/E/F/H/K/M/N/Q/R/S/T/V/Y); |
| | | (F/L);(A/H/K/L/M/R/S/T/Y);X;X;(F/G) [Motif 11 (SEQ ID NO: 218)] |
| 219 | | (A/I/L/M/N/V);(F/H/L/Q/Y);(A/C/E/I/L/M/S/T);R;(A/I/P/S/V);L;G;(D/K/N;I/T/V); |
| | | (F/I/L/M/V);Y [Motif 12 (SEQ ID NO: 219)] |
| 220 | | (A/C/G/S);(F/Y);H;G;(D/E);T;(F/I/L/M/V/W);(A/D/E/G/K/M/Q);(A/C/G/P/T/V);(I/L/M/V); |
| | | (A/D/E/N/S);(A/C/I/L/M/T/V) [SEQ ID NO: 220] |
| 221 | | (F/Y);(F/L/Y);(A/C/N/S/V);D;(D/N/S);G;(A/S);(A/C/E/I/S/T/V);(A/C/G/S);(C/I/M/T/V); |
| | | (D/E);(C/I/V);(A/G/S);(I/L/M/V);K;(A/I/M/V);(A/C/S/V) [SEQ ID NO: 221] |
| 222 | | W;(H/Y/W);(A/E);F;(A/Q/S/T);Q;(H/Q/V);X;X;X (SEQ ID NO: 222) |
| 223 | | W;(H/Y/W);P;(A/C/D/E/I/K/M/N/Q/S/T/V);(A/Q/S/T);Q;(H/Q/V);X;X;X (SEQ ID NO: 223) |
| 224 | | W;(H/Y/W);P;F;(A/Q/S/T);(H/S);(H/Q/V);X;X;X (SEQ ID NO: 224) |
| 225 | | W;(H/Y/W);P;F;(A/Q/S/T);Q;(H/Q/V);X;X;(A/E/L/N/T) (SEQ ID NO: 225) |
| 226 | | W;(H/Y/W);(A/E);(A/C/D/E/I/K/M/N/Q/S/T/V);(A/Q/S/T);(H/S);(H/Q/V);X;X; |
| | | (A/E/L/N/T) (SEQ ID NO: 226) |
| 227 | | (I/L/V);(D/E);(S/G);(R/A);X;(A/D/G/K);(E/D/N);X;L (SEQ ID NO: 227) |
| 228 | | (F/I/L/V/Y);D;(A/G);(D/M/T);(A/G/S);S;W;W;(T/S/V);(I/Q) (SEQ ID NO: 228) |
| 229 | | (F/I/L/V/Y);D;(A/G);(C/I/P/S);F;S;W;W;(T/S/V);(I/Q) (SEQ ID NO: 229) |
| 230 | | (F/I/L/V/Y);D;(A/G);(C/I/P/S);(A/G/S);C;W;W;(T/S/V);(I/Q) (SEQ ID NO: 230) |
| 231 | | (F/I/L/V/Y);D;(A/G);(C/I/P/S);(A/G/S);S;(F/L/M);W;(T/S/V);(I/Q) (SEQ ID NO: 231) |
| 232 | | (F/I/L/V/Y);D;(A/G);(C/I/P/S);(A/G/S);S;W;(A/C/D/G/M/S);(T/S/V);(I/Q) (SEQ ID NO: 232) |
| 233 | | (F/I/L/V/Y);D;(A/G);(C/I/P/S);(A/G/S);S;W;W;V;(I/Q) (SEQ ID NO: 233) |
| 234 | | (F/I/L/V/Y);D;(A/G);(D/M/T);F;C;(F/L/M);(A/C/D/G/M/S);V;(I/Q) (SEQ ID NO: 234) |
| 235 | | (F/Y);(G/D);(H/Q);(A/I/V);I;(F/L/Y);(A/L/P);(E/G/H/L/N/S/T) (SEQ ID NO: 235) |
| 236 | | (F/Y);(G/D);(H/Q);(A/I/V);(M/I/L);I;(A/L/P);(E/G/H/L/N/S/T) (SEQ ID NO: 236) |
| 237 | | (F/Y);(G/D);(H/Q);(A/I/V);(M/I/L);(F/L/Y);(A/E/G/L/W);(E/G/H/L/N/S/T) (SEQ ID NO: 237) |
| 238 | | (F/Y);(G/D);(H/Q);(A/I/V);I;I;(A/E/G/L/W);(E/G/H/L/N/S/T) (SEQ ID NO: 238) |
| 239 | | (A/G/T);(F/L/T/Q/V);X;(G/N/D/E/R);A;Y;H;G;D;T;(L/I/M);(G/S);(A/C/S/T/V);(M/L/T); |
| | | (D/E/N);X;(A/E/K/Q/R/S/T);(A/E/I/Q/V/T);(E/G/I/K/P/S);(C/E/N/S/T);X;(F/T/Y); |
| | | (M/N/S/T);X (SEQ ID NO: 239) |
| 240 | | (A/G/T);(F/L/T/Q/V);X;(G/N/D/E/R);(S/C/G);Y;S;G;D;T;(L/I/M);(G/S);(A/C/S/T/V); |
| | | (M/L/T);(D/E/N);X;(A/E/K/Q/R/S/T);(A/E/I/Q/V/T);(E/G/I/K/P/S);(C/E/N/S/T);X;(F/T/Y); |
| | | (M/N/S/T);X (SEQ ID NO: 240) |
| 241 | | (A/G/T);(F/L/T/Q/V);X;(G/N/D/E/R);(S/C/G);Y;H;G;D;T;(L/I/M);(G/S);(A/C/S/T/V); |
| | | (M/L/T);(D/E/N);X;A;(A/E/I/Q/V/T);(E/G/I/K/P/S);(C/E/N/S/T);X;(F/T/Y);(M/N/S/T); |
| | | X (SEQ ID NO: 241) |
| 242 | | (A/G/T);(F/L/T/Q/V);X;(G/N/D/E/R);(S/C/G);Y;H;G;D;T;(L/I/M);(G/S);(A/C/S/T/V); |
| | | (M/L/T);(D/E/N);X;(A/E/K/Q/R/S/T);(K/S);(E/G/I/K/P/S);(C/E/N/S/T);X;(F/T/Y); |
| | | (M/N/S/T);X (SEQ ID NO: 242) |
| 243 | | (A/G/T);(F/L/T/Q/V);X;(G/N/D/E/R);(S/C/G);Y;H;G;D;T;(L/I/M);(G/S);(A/C/S/T/V); |
| | | (M/L/T);(D/E/N);X;(A/E/K/Q/R/S/T);(A/E/I/Q/V/T);(DELETION/L/T);(C/E/N/S/T);X; |
| | | (F/T/Y);(M/N/S/T);X (SEQ ID NO: 243) |
| 244 | | (A/G/T);(F/L/T/Q/V);X;(G/N/D/E/R);(S/C/G);Y;H;G;D;T;(L/I/M);(G/S);(A/C/S/T/V); |
| | | (M/L/T);(D/E/N);X;(A/E/K/Q/R/S/T);(A/E/I/Q/V/T);(E/G/I/K/P/S); |
| | | (A/C/D/E/F/G/H/I/K/L/M/N/Q/R/S/T/V/W/Y);X;(F/T/Y);(M/N/S/T);X (SEQ ID NO: 244) |
| 245 | | (A/G/T);(F/L/T/Q/V);X;(G/N/D/E/R);(S/C/G);Y;H;G;D;T;(L/I/M);(G/S);(A/C/S/T/V); |
| | | (M/L/T);(D/E/N);X;(A/E/K/Q/R/S/T);(A/E/I/Q/V/T);(E/G/I/K/P/S);(C/E/N/S/T); |
| | | (A/C/E/L/Q/V);(F/T/Y);(M/N/S/T);X (SEQ ID NO: 245) |
| 246 | | (A/G/T);(F/L/T/Q/V);X;(G/N/D/E/R);(S/C/G);Y;H;G;D;T;(L/I/M);(G/S);(A/C/S/T/V); |
| | | (M/L/T);(D/E/N);X;(A/E/K/Q/R/S/T);(A/E/I/Q/V/T);(E/G/I/K/P/S);(C/E/N/S/T);X; |
| | | (C/D/E/F/H/I/K/M/P/Q/R/V/W);(M/N/S/T);X (SEQ ID NO: 246) |
| 247 | | (A/G/T);(F/L/T/Q/V);X;(G/N/D/E/R);(S/C/G);Y;H;G;D;T;(L/I/M);(G/S);(A/C/S/T/V); |
| | | (M/L/T);(D/E/N);X;(A/E/K/Q/R/S/T);(A/E/I/Q/V/T);(E/G/I/K/P/S);(C/E/N/S/T);X; |
| | | (F/T/Y);(C/D/G/I/N/Q/R/V/W);X (SEQ ID NO: 247) |
| 248 | | (A/G/T);(F/L/T/Q/V);X;(G/N/D/E/R);(S/C/G);Y;H;G;D;T;(L/I/M);(G/S);(A/C/S/T/V); |
| | | (M/L/T);(D/E/N);X;(A/E/K/Q/R/S/T);(A/E/I/Q/V/T);(E/G/I/K/P/S);(C/E/N/S/T);X; |
| | | (F/T/Y);(M/N/S/T);(DELETION/A/L/P) (SEQ ID NO: 248) |
| 249 | | (A/G/T);(F/L/T/Q/V);X;(G/N/D/E/R);A;Y;S;G;D;T;(L/I/M);(G/S);(A/C/S/T/V);(M/L/T); |
| | | (D/E/N);X;A;(K/S);(DELETION/L/T);(A/C/D/E/F/G/H/I/K/L/M/N/Q/R/S/T/V/W/Y); |
| | | (A/C/E/L/Q/V);(C/D/E/F/H/I/K/M/P/Q/R/V/W);(C/D/G/I/N/Q/R/V/W);(DELETION/A/L/P) |
| | | (SEQ ID NO: 249) |
| 250 | | C;(E/H/I/Q/V/T);(D/E/H/P);(S/W);(F/H/Y);X;(G/P/Q/R/S);(E/K/Q/R/W) (SEQ ID NO: 250) |
| 251 | | (A/E/K/Q/R/S/T);(D/F/H/I/M/T/W/Y);(D/E/H/P);(S/W);(F/H/Y);X;(G/P/Q/R/S); |
| | | (E/K/Q/R/W) (SEQ ID NO: 251) |
| 252 | | (A/E/K/Q/R/S/T);(E/H/I/Q/V/T);(D/E/H/P);(S/W);(N/W);X;(G/P/Q/R/S); |
| | | (E/K/Q/R/W) (SEQ ID NO: 252) |
| 253 | | C;(D/F/H/I/M/T/W/Y);(D/E/H/P);(S/W);(N/W);X;(G/P/Q/R/S);(E/K/Q/R/W) |
| | | (SEQ ID NO: 253) |
| 254 | | (F/S/W);(F/I/L/P);X;A;(P/S);X;(I/L/M/T/V) (SEQ ID NO: 254) |
| 255 | | (A/E/I);(A/S);(A/G);G;M;X;(F/M/L);(A/C/I/V) (SEQ ID NO: 255) |
| 256 | | (A/G);(C/F/H/I/K/M/N/R/T/V/W/Y);(A/G);G;M;X;(F/M/L);(A/C/I/V) (SEQ ID NO: 256) |
| 257 | | (A/G);(A/S);H;G;M;X;(F/M/L);(A/C/I/V) (SEQ ID NO: 257) |
| 258 | | (A/G);(A/S);(A/G);G;L;X;(F/M/L);(A/C/I/V) (SEQ ID NO: 258) |
| 259 | | (A/E/I);(C/F/H/I/K/M/N/R/T/V/W/Y);H;G;L;X;(F/M/L);(A/C/I/V) (SEQ ID NO: 259) |
| 260 | | L;L;H;(A/C/S);H;S;(F/Y);(N/S/T);(A/G/T);(H/N/Y) (SEQ ID NO: 260) |
| 261 | | L;L;H;(G/R);H;S;(F/Y);(H/P);(A/G/T);(H/N/Y) (SEQ ID NO: 261) |
| 262 | | L;L;H;(A/C/S);H;S;(F/Y);(H/P);(A/G/T);(H/N/Y) (SEQ ID NO: 262) |
| 263 | | (A/E/G/H/K/P/Q/S/V);(A/C/G/K/N/Q/R/S/T/V/Y);(H/I/L/V);(A/D/E/H/K/L/N/Q/R/S); |
| | | (S/K);(A/C/I/L/T/V);(F/I/M/T/V/W);(A/D/G/N/S/V;(I/L/M/Q/V) (SEQ ID NO: 263) |
| 264 | | (A/I/L/V/Y);S;(A/D/E/I/K/L/M/N/T/Q/R/S);(A/D/E/F/H/K/M/N/Q/R/S/T/V/Y); |
| | | (F/L);(A/H/K/L/M/R/S/T/Y);X;X;(F/G) (SEQ ID NO: 264) |
| 265 | | (A/I/L/V/Y);(A/I/L/N/Q/R/V);(A/D/E/I/K/L/M/N/T/Q/R/S); |
| | | (A/D/E/F/H/K/M/N/Q/R/S/T/V/Y);(F/L);C;X;X;(F/G) (SEQ ID NO: 265) |
| 266 | | (A/I/L/V/Y);S;(A/D/E/I/K/L/M/N/T/Q/R/S);(A/D/E/F/H/K/M/N/Q/R/S/T/V/Y); |
| | | (F/L);C;X;X;(F/G) (SEQ ID NO: 266) |
| 267 | | (A/I/L/M/N/V);(F/H/L/Q/Y);(A/C/E/I/L/M/S/T);Q;(A/I/P/S/V);L;G; |
| | | (D/K/N;I/T/V);(F/I/L/M/V);Y (SEQ ID NO: 267) |
| 268 | | (A/G/S);(F/Y);H;G;(D/E);T;(F/I/L/M/V/W);(A/D/E/G/K/M/Q);(A/G/P/T);(I/L/M/V); |
| | | (A/E/S);(A/I/L/T/V) (SEQ ID NO: 268) |
| 269 | | D;E;(I/V);(A/F/L/M);(T/V);G;(F/L/W);G;(K/R);(C/S/T);G;(A/E/K/L/Q/P/R/S/T); |
| | | (F/L/M/N/R/W);F;(A/G/S) (SEQ ID NO: 269) |
| 270 | | (H/Q);(C/I/M/S/T/V);(I/L/M/T/V);(F/L/Y);(A/G/L/P);(D/E/G/M/N); |
| | | (A/C/F/I/L/M/N/Y);(A/G/I/L/S/T/V);(H/N/Y);(E/K/N/P/Q/S/V);(A/G/K/Q/P/T) |
| | | (SEQ ID NO: 270) |
| 318 | Arabidopsis thaliana | MIPVTATLIRHRLRHLRHRIRFKSTSVSPFHLPLNHPTYLIWSANTSLGKTLVSTGIAASFLLQQPSSSATKLLYLKPIQTGFPSDSDSRFVFSKL |
| | BioDA protein P421G | DSLSLRRQIPISISNSVLHSSLPAAKSLGLNVEVSESGMCSLNFRDEKTVTGAPELLCKTLYAWEAAISPHLAAERENATVEDSVVLQMIEKCLK |
| | (SEQ ID NO: 318) | EEMECGVKSEKSDLLCLVETAGGVASPGPSGTLQCDLYRPFRLPGILVGDGRLGGISGTIAAYESLKLRGYDIAAVVFEDHGLVNEVPLTSYL |
| | | RNKVPVLVLPPVPKDPSDDLIEWFVESDGVFKALKETMVLANLERLERLNGMAKLAGEVFWWPFTQHKLVHQETVTVIDSRCGENFSIYKAS |
| | | DNSSLSQQFDACASWWTQGPDPTFQAELAREMGYTAARFGHVMFGENVYEPALKCAELLLDGVGKGWASRVYFSDNGSTAIEIALKMAFR |
| | | KFCVDHNFCEATEEEKHIVVKVIALRGSYHGDTLGAMEAQAPSPYTGFLQQPWYTGRGLFLDPPTVFLSNGSWNISLPESFSEIAPEYGTFTS |
| | | RDEIFDKSRDASTLARIYSAYLSKHLQEHSGVRQSAHVGALIIEPVIHGAGGMHMVDPLFQRVLVNECRNRKIPVIFDEVFTGFWRLGVETTTE |
| | | LLGCKPDIACFAKLLTGGMVPLAVTLATDAVFDSFSGDSKLKALLHGHSYSAHAMGCATAAKAIQWFKDPETNHNITSQGKTLRELWDEELVQ |
| | | QISSHSAVQRVVVIGTLFALELKADASNSGYASLYAKSLLIMLREDGIFTRPLGNVIYLMCGPCTSPEICRRLLTKLYKRLGEFNRT |
| 319 | Selaginella moellendorffii | MLLLALWRRLGRRSSAAGHVRCFSSAQALPSAEDFAARRREDALLGEIDARYPAFMVWGCNTDVGKTLVSTGMAAALLSPGKSQKSHVLYV |
| | BioDA (SEQ ID NO: 319) | KPVQTGFPDDSDARSVTQRIDSLVGRSESIFLSNRTMWLSDEASKKAALTKRSTDSSGSWKNSANSVKSSSSLTCETLWSWCAAVSPHLAA |
| | | QQEGGGASDDELRKVLRSSLWEFGAGASQTGRSWAFVETAGGVASPAPSGTLQCDLYRPLRLPGILVGDGRLGGISTTLAAYESLLLRGYE |
| | | TICIVIQDMGLGNNQHLSKQLHNRVPVFVIPAVPKNPGDDLAGWFHDTSSTFVQVVSLLEDAYHKRIQRLREMPKKASELLWWPFTQHRLVP |
| | | EENVTVIDSRSGEFFSVYENKHGRFAQQFDACASWWTQGPDASLQHELAKQIAYAAGRYGHVMFPENVHEPGLRCAELLLQGVGRGWAH |
| | | RVYISDNGSTATEIALKMALRKYAVDHGFLTNQGDKKPDFKVLALRGSYHGDTLGAMEAQAPSPYTGFSQQPWYSGRGVFLDPPTVCCQNR |
| | | KWQLRVPERYGTIATELAWDAREDVFALGRDNTELAKIYSSAIEKEIMSSQGSLGGQTIAGLIIEPIIHAAGGMELVDPLFQRILTQQCKCHKIPVI |
| | | FDEVFTGCWRLGAESAWELLGCKPDIACYAKLLTGGTVPLAATLATQDIFQAFEGASKLDALLHGHSYTAHPVGCSAAVTALEWFKDPSKNP |
| | | NLLPGTSRLQEQWDPELVAEISSRSSVERVISLGTLMALELRSTSSSQKGYSSLLSSGVVKELRQELGIYTRPLGNVVYFMCGPLTKPSVCSS |
| | | LLRRMLKVLE |
| 320 | Selaginella | MSSAQALPSAEDFAARRREDALLGEIDARYPAFMVWGCNTDVGKTLVSTGMAAALLSPGKSQKSHVLYVKPVQTGFPDDSDARSVTQRIDS |
| | moellendorcffii | LVGRSESIFLSNRTMWLSDEASKKAALTKRSTDSSGSWKNSANSVKSSSSLTCETLWSWCAAVSPHLAAQQEGGGASDDELRKVLRSSLW |
| | BioDAΔMTP (SEQ ID | EFGAGASQTGRSWAFVETAGGVASPAPSGTLQCDLYRPLRLPGILVGDGRLGGISTTLAAYESLLLRGYETICIVIQDMGLGNNQHLSKQLHN |
| | NO: 320) | RVPVFVIPAVPKNPGDDLAGWFHDTSSTFVQVVSLLEDAYHKRIQRLREMPKKASELLWWPFTQHRLVPEENVTVIDSRSGEFFSVYENKHG |
| | | RFAQQFDACASWWTQGPDASLQHELAKQIAYAAGRYGHVMFPENVHEPGLRCAELLLQGVGRGWAHRVYISDNGSTATEIALKMALRKYA |
| | | VDHGFLTNQGDKKPDFKVLALRGSYHGDTLGAMEAQAPSPYTGFSQQPWYSGRGVFLDPPTVCCQNRKWQLRVPERYGTIATELAWDAR |
| | | EDVFALGRDNTELAKIYSSAIEKEIMSSQGSLGGQTIAGLIIEPIIHAAGGMELVDPLFQRILTQQCKCHKIPVIFDEVFTGCWRLGAESAWELLG |
| | | CKPDIACYAKLLTGGTVPLAATLATQDIFQAFEGASKLDALLHGHSYTAHPVGCSAAVTALEWFKDPSKNPNLLPGTSRLQEQWDPELVAEIS |
| | | SRSSVERVISLGTLMALELRSTSSSQKGYSSLLSSGVVKELRQELGIYTRPLGNVVYFMCGPLTKPSVCSSLLRRMLKVLE |
| 321 | Ostreococcus tauri | MLGSASDGAASGNGRALELSIASFAIWGANTGVGKTLASAGLARAATRRGTATTFLKPVQTGYPEDSDAGFVARQTKGNETAGEHAAVASG |
| | ATCC 22665 | STEGGGEGFAAGVTRAHTEFAWRRAAGPHLTAAEEGRPVSDEEIVRATRNYLREFAESAREGNARGFAVIETAGGVASPGPSGTLQCEILRA |
| | BioDAΔMTP A374D | LRLPAILIGDGRLGGISTTTSAYESLHARGYDVVGVLMADDGYANHEAVAKILPSGTKVFALPPLPPQGKADAWLEKSAGVFSEAFETAMNWH |
| | (SEQ ID NO: 321) | EKRIQALQKLPGEALEKIWWPFTQHATVEPDTVTVIDGRYGEDFEIYDQEKGRDGAIDLRFDGDASWWTQGVSAELQQELNSAAANAAGRY |
| | | GHVMFPENVHQPSMDATNALLEGPGKGWGSRVFYSDNGSTATEVGLKMAFRKYYVDAGLVSKDGHQRAKDLINDGSCHLPQLRIIALDGSY |
| | | HGDTLGAMDMQSPSVFTGPMQTPWYQPRGLFLQPPSLAIRNGEWTVALPPHGLLNDGEDRLFHDGKDLESNTTWKSKADAFDIGKRLDSP |
| | | LANEYRQAVDKALERAEQDAKAGHMGPVGALMMEPVLHGAGGMVLIDPLFQNILMKVCRERKIPIVLDEVFAGIWRLGTEGAWEIIGEKPDIS |
| | | CYAKLLTGGLMPMSATVATADVFDVFYGPGKAQGLLHGHSYTAYPIGCAVASKALSLYKDPAKNPNLIVDASSKGDTPTAYLRELWDESKLK |
| | | EISNKSSVRRVFGQGCVLAVELEVEGEGGYSSNAAKEVVLRLRPHSVQARPLGNVVYLMCAPTTPKATCDRLLNVLAEELSE |
| 322 | Ostreococcus tauri | MLGSASDGAASGNGRALELSIASFAIWGANTGVGKTLASAGLARAATRRGTATTFLKPVQTGYPEDSDAGFVARQTKGNETAGEHAAVASG |
| | ATCC 22665 | STEGGGEGFAAGVTRAHTEFAWRRAAGPHLTAAEEGRPVSDEEIVRATRNYLREFAESAREGNARGFAVIETAGGVASPGPSGTLQCEILRA |
| | BioDAΔMTP F332A | LRLPAILIGDGRLGGISTTTSAYESLHARGYDVVGVLMADDGYANHEAVAKILPSGTKVFALPPLPPQGKADAWLEKSAGVFSEAFETAMNWH |
| | (SEQ ID NO: 322) | EKRIQALQKLPGEALEKIWWPATQHATVEPDTVTVIDGRYGEDFEIYDQEKGRDGAIDLRFDGAASWWTQGVSAELQQELNSAAANAAGRY |
| | | GHVMFPENVHQPSMDATNALLEGPGKGWGSRVFYSDNGSTATEVGLKMAFRKYYVDAGLVSKDGHQRAKDLINDGSCHLPQLRIIALDGSY |
| | | HGDTLGAMDMQSPSVFTGPMQTPWYQPRGLFLQPPSLAIRNGEWTVALPPHGLLNDGEDRLFHDGKDLESNTTWKSKADAFDIGKRLDSP |
| | | LANEYRQAVDKALERAEQDAKAGHMGPVGALMMEPVLHGAGGMVLIDPLFQNILMKVCRERKIPIVLDEVFAGIWRLGTEGAWEIIGEKPDIS |
| | | CYAKLLTGGLMPMSATVATADVFDVFYGPGKAQGLLHGHSYTAYPIGCAVASKALSLYKDPAKNPNLIVDASSKGDTPTAYLRELWDESKLK |
| | | EISNKSSVRRVFGQGCVLAVELEVEGEGGYSSNAAKEVVLRLRPHSVQARPLGNVVYLMCAPTTPKATCDRLLNVLAEELSE |
| 323 | Ostreococcus tauri | MLGSASDGAASGNGRALELSIASFAIWGANTGVGKTLASAGLARAATRRGTATTFLKPVQTGYPEDSDAGFVARQ |
| | ATCC 22665 | TKGNETAGEHAAVASGSTEGGGEGFAAGVTRAHTEFAWRRAAGPHLTAAEEGRPVSDEEIVRATRNYLREFAES |
| | BioDAΔMTP P407A | AREGNARGFAVIETAGGVASPGPSGTLQCEILRALRLPAILIGDGRLGGISTTTSAYESLHARGYDVVGVLMADDGY |
| | (SEQ ID NO: 323) | ANHEAVAKILPSGTKVFALPPLPPQGKADAWLEKSAGVFSEAFETAMNWHEKRIQALQKLPGEALEKIWWPFTQH |
| | | ATVEPDTVTVIDGRYGEDFEIYDQEKGRDGAIDLRFDGAASWWTQGVSAELQQELNSAAANAAGRYGHVMFAEN |
| | | VHQPSMDATNALLEGPGKGWGSRVFYSDNGSTATEVGLKMAFRKYYVDAGLVSKDGHQRAKDLINDGSCHLPQ |
| | | LRIIALDGSYHGDTLGAMDMQSPSVFTGPMQTPWYQPRGLFLQPPSLAIRNGEWTVALPPHGLLNDGEDRLFHD |
| | | GKDLESNTTWKSKADAFDIGKRLDSPLANEYRQAVDKALERAEQDAKAGHMGPVGALMMEPVLHGAGGMVLIDP |
| | | LFQNILMKVCRERKIPIVLDEVFAGIWRLGTEGAWEIIGEKPDISCYAKLLTGGLMPMSATVATADVFDVFYGPGKA |
| | | QGLLHGHSYTAYPIGCAVASKALSLYKDPAKNPNLIVDASSKGDTPTAYLRELWDESKLKEISNKSSVRRVFGQGC |
| | | VLAVELEVEGEGGYSSNAAKEVVLRLRPHSVQARPLGNVVYLMCAPTTPKATCDRLLNVLAEELSE |
| 324 | Zea mays BioDA C372D | MVRRLFLHHARRRLHSGTASSIPLSTPIFAIFGANTGVGKTLVSAGLATALLSSPSPAVSSVAYLKPLQTGYPVDSDASFVFSRTPALLRAFPPC |
| | amino acid (SEQ ID NO: | RATRLIASCRTFFQSPAVEAKAESLHESQEKVVTYGAGAAEQTKVLACCTAYAWREPVSPHLAAEREGMAAGDDEVKGCVEQWLLEEGIGE |
| | 324) | GGEVWKILETAGGVASPSASGTLQCDLYRPFRLPAILVGDGRLGGISSTLSAYETLLLRGYDVSAVILEDRELSNDKFLLSYLRNRVHVLILPQI |
| | | PEDPLDDLTDWFSESSSIFIFLKDELQSFHSRRIERLNSMQRKSKALLWWPFTQHNLVPQDSVTVIDSRYGENFSVYKIKDKMLIPQFDADAS |
| | | WWTQGPDSNLQIELARDMGYAAARYGHVMFPENVHEPALHSAEVLLGGVGKGWASRVYYSDNGSTAIEIALKMAFRKFSLDHGIMASSENS |
| | | TRNERNIQLKVLALNGSYHGDTLGAMEAQAPSAYTSFLQQPWYSGRGLFLDPPTVYIKSEVYNISLPQSMQHDPQTYGFFSQAEVFCKTRDK |
| | | TFAADLYSSYIKQKLSEFSLSSSSEHLAALIIEPVIQGAGGMLMIDPLFQRVLVSECRSRKIPVIFDEVFTGFWRLGVESASELLGCLPDIACYAK |
| | | LMTGGIIPLAATLATEEVFESFESDSKLTALLHGHSYTAHAMGCTAALKAMQWYRGPSTNSNLDTDHMKLKELWDGTLVKQLSSLPNVKRVV |
| | | SLGTLCAIELKAEGADAGYASLYASSLVQQLRKEDDIYIRPLGNVIYLMCGPCTPRDSCTRQLLKVHRRLCALN* |
| 325 | Pantoea ananatis BioDA | MSVSCCCRNLGKTIKKAIPSHHLHLRSLGGSLYRRIQSSSMFTSQDAEFDRQHIWHPYTSMQQPLPCYPVVAAKDYHLQLADGRTLVDGMSS |
| | with MTP (SEQ ID NO: | WWAAIHGYNHPRLNRALQQQMGQMSHVMFGGITHPAAVALCRQLVSMTPDALECVFLADSGSVAVEVAMKMALQYWLGRGETRQQFLTL |
| | 325) | KRGYHGDTFAAMSVCDPDNSMHSLWRGYLPEHHFAAAPQCGFDDEWDARDAEDFIRLIQQHRGQLAAVILEPIVQGAGGMRFYHPRYLQC |
| | | VREACDRHGILLIADEIATGFGRTGTLFACEHAAISPDIMCVGKALTGGTMSLAATLTTRDVADTISLSPAGCFMHGPTFMGNPLACAVASESL |
| | | AMVQEGDWRQQVAAIERQLRGALLPLRHAPAVADVRVLGAIGVVETHQPVNMAALQQFFVSQGVWIRPFGRLIYLMPPYIISPEALDKLVNAI |
| | | GAALEQPAHFLS |
| 326 | Stenotrophomonas | MSVSCCCRNLGKTIKKAIPSHHLHLRSLGGSLYRRIQSSSMLADPTPSPLAHHWRQRDLQVLWHPCTQMREHPDTLPLVPIARGEGAWLIDH |
| | maltophilia BioDA with | DGNRYLDAVSSWWTNLFGHAEPRIGGAIAAQATQLEQVMLAGFGHEPAITLAERLLALAPRQPGREPLAKVFYADNGSAGVEVALKMAFQYF |
| | MTP (SEQ ID NO: 326) | QNRGESRRTRFIALENGYHGETLGALALGDIPLYRRVYAPLLAEALFAPSPDAYLAEPGQSAADRARQAADGLATLFDQHPGEICAVILEPRLQ |
| | | CAGGMRMHDPVYLQRVRELCDAHGAFMIADEIATGFGRTGTLFACEQAGVMPDLMCLSKGLTGGFLPLAAVLATQALYDAFLDDSRERAFL |
| | | HSHSYTGNPLACAAALTTLDIFRDDDVIARNRSIASVMGTLAAPFNDHPHVADVRQAGMVVAFELSRDGNKRTPFDPAMRLGLHAYKAALKR |
| | | GVVLRPLGDVLYWMPPYCVDDEQLELLAHTTLAAIDEAIACA |
| 327 | Chroococcidiopsis sp. | MSVSCCCRNLGKTIKKAIPSHHLHLRSLGGSLYRRIQSSSMSVEEIHPSYPIWHPSTQMKTSPPPLKVVKGQGVMLELEDGRQILDCISSWWWV |
| | CCMEE 29 BioDA with | TIHGHSHPILANALYQQAQQLEHVIFAGFTHEPAEQLARKLLNHLPKSLTRVFFSDNGSTAVEVALKMAYQYWYNQGETQRTSFIGFEGGYHG |
| | MTP (SEQ ID NO: 327) | DTLGAMSVGGSTSWWQTFQLLMFSIDVVPFPATFDRDPNVEAHEAQSLETITRLLKQNPDRYAGIFIEPLVQGAAGMRLCRPQFLQELESLA |
| | | RSFDVLLIYDEVMTGFGRTGEFFACLKSATTPDILCLSKGLSGGCLPLAVTLATEDIYRAFYSDDIHKAFLHGHSYTGNPLACATGVASLELLEQ |
| | | NPEFRAIEHQHRYYLEKWLIGHPKVEQLRTCGTIAAMEVRTEDQSSYFNAIGPTLRSRFLEEGFLLRPLGNTIYIMPPYCITPNQLESIYQAIRRV |
| | | LDTL |
| 328 | Bacillus subtilis BioDA | MSVSCCCRNLGKTIKKAIPSHHLHLRSLGGSLYRRIQSSSMKQSMIEKSKKHLWLPFTQMKDYDEDPLIIESGNGIMLKDISGKEYYDGFSSVW |
| | with MTP (SEQ ID NO: | LNVHGHRKKEMDEAIQKQLEKIAHSTLLGMTNVPATELAEMLTGITPEKLTRVFYSDSGAEAMEIALKMAFQYWKNIGRPEKQTFISMQNGYH |
| | 328) | GDTIGAVSVGSIELFHHVYGPLMFESYKAPIPYVYRSESGDPVQCRDECLHALEQLLAKHHEEIAALTIESMVQGASGMIVMPEGFLAGVRKLC |
| | | TAYDVLMIVDEVATGFGRTGKMFACEHENVQPDLMAAGKGITGGYLPIAVTFATEAIYEAFYDDYDKMKTFFHGHSYTGNQLGCAAAIENLRL |
| | | FESERIVEQVAEKSKTAAEFLHDLKQLSHVGDVRQLGFMCGIELVQDKGTKQPYPPEKRMGYKVSLKMRELGMLTRPLGDVIAFLPPLASTTD |
| | | QLQAMISIMKEAIAEVTGRGF |
| 329 | Streptomyces | MSVSCCCRNLGKTIKKAIPSHHLHLRSLGGSLYRRIQSSSMPDLSVPELLALDRRHVWHPYGPMPGRIDPLVVESASGVRLKLAGGSGELVD |
| | viridochromogenes | GMSSWWSAIHGYNHPVLNEAAHEQLSRMSHVMFGGLTHEPAVRLAKLLVDMSPDGLEHVFLADSGSVSVEVAVKMCLQYWRSLGRPGKQ |
| | BioDA with MTP (SEQ | RLLTWRGGYHGDTWQPMSVCDPEGGMHELWTGVLQRQVFADPPPAVYDESYSDHLRSLIERHAHELAAVIVEPVVQGAGGMRFHSPAYL |
| | ID NO: 329) | RVLREACDAHDVLLVFDEIATGFGRTGALFAADHAAVTPDVMCMGKALTGGYLTLAATVCTSRVADGISRGEVPVLAHGPTFMGNPLAAAVA |
| | | CASIELLLGQDWLAEVKRIEAGLREGLAPVADLPGVRDVRVLGAIGVVQLDHAVDMKAATDAAVREGVWLRPFRDLIYTMPPYVTGDADLARI |
| | | ARAVCAAAREG |
| 330 | Pedobacter hartonius | MSVSCCCRNLGKTIKKAIPSHHLHLRSLGGSLYRRIQSSSMTLTQRDAESIWHPYTQQLNARPPLPVLSGKGAYLYDEEGCRYIDAVSSWWWV |
| | BioDA with MTP (SEQ | TLHGHSHPYIVERVTAQLQQLDQVIFAGFTHEPAIALAENLLAILPEGQKKVFYTDNGSTAVEVALKMCVQYWFNKGKSRKKVLAFNNGYHGD |
| | ID NO: 330) | TFGAMSVSGRSAWTAPFDNLLFEVIFIDTPTEENLPVLKEIISVHAAELACFVYEPLVQGSAGMLMYEAPALDQLMAHCLEEGVLMIQDEVFTG |
| | | FGRTGKNFAADHLKTQPDVMCFSKGLTGGTMPLGVTTCTEDIYNAFLSEDKLKTLFHGHSFTANPLACTAALASIELLLQHEAQDNIRRICNRH |
| | | AEFSLKICGHQKVAAVRQTGTIIAIEWQTESGTSYFSKLRNLLYDYFMDKGILMRPLGNVIYILPPYCISDEDLKKIYQEIQNALEIF |
| 331 | Chitinophaga filiformis | MSVSCCCRNLGKTIKKAIPSHHLHLRSLGGSLYRRIQSSSMLWYPYTQMRQINELPKMVSGEGVMMHLEDGRSLIDGISSWWAVIHGYNHPA |
| | BioDA with MTP (SEQ | LNAALLTQANKFAHVMLGGMTHNPALDLAAKLVSITPQGLNHVFFSDSGSIGVEVALKMSIQYWKNIGYNGKSKIISLRNGYHGDTFKAMEVS |
| | ID NO: 331) | DDSDFTRAFSDVLRRGYILDIPEGGFDADAALVKQATDKLEALLRREHHNIAAFIVEPIVQCAGGFNIYSPLYLKAARELCTKYNVLFVFDEVAT |
| | | GFGRTGKLFAAEHAGVTPDIMILGKALTAGYMGHAATLATSAVFDSFLGDNYEKALMHGPTFMANPLACAVALRSISIIEDEQYLQKIAKIQSIIR |
| | | EQFDTFSSPAIVAKRSIGAIGALEMKDAACLSGFKEFSQQRGVWLRPIGNVLYLMPPYIISEKELLTILQVIKDWIQQIK |
| 332 | Tenacibaculum | MSVSCCCRNLGKTIKKAIPSHHLHLRSLGGSLYRRIQSSSMNLTERDKKHLWHPLTQHQLYPNHLAITKAKGAILYDDKNNEYIDGIASWYTCM |
| | adriaticum BioDA with | YGHCNEYITSSVYKQMQQLDHVVFAGFTHEPAIKLSEELIKILPENQEKVFFSDNGSTSVDVAIKMALQYHFNQGEKKGKIIALEDGFHGDTFG |
| | MTP (SEQ ID NO: 332) | AMSVSGLSVYNGPFEDFFIDVARIPVPTQENFEHVKEEFIKLITEHNVAAFVYEPLVQGAAAMKMFDGNLLNELIKIAQENNVLAIADEVMTGFG |
| | | KTGKNFASEYLEHKPDIICLSKSLTAGLVPMAITSCSQKVYNAFLSDDIGKGFFHGHTYSANPVACSAALAGIELLQSEEIQGNIQRIIKSHQEFD |
| | | SKIKKHPKVKSTRQKGIIYALDLNVEMERYGDLRYKLFNFFMENGVCLRPLGNTIYILAPFVITNEQLEKIYKTIEKALEIL |
| 333 | Pantoea ananatis BioA | ATGAGCGTTAGCTGTTGTTGCCGTAATTTGGGTAAAACTATTAAGAAAGCAATTCCCTCTCATCACCTACACCTTAGGTCTTTGGGTGGG |
| | nucleic acid with MTP | TCTTTGTACCGGCGAATTCAGAGCTCTAGCATGTTTACTTCACAGGACGCTGAATTTGATAGACAGCATATTTGGCACCCTTACACGTCC |
| | (SEQ ID NO: 333) | ATGCAACAGCCATTGCCTTGCTATCCAGTGGTTGCTGCTAAAGACTACCACCTACAACTGGCTGATGGACGGACATTGGTTGATGGGAT |
| | | GTCATCATGGTGGGCTGCTATTCACGGATATAATCATCCCAGACTTAATCGGGCCCTGCAACAACAAATGGGTCAGATGTCCCACGTTA |
| | | TGTTCGGAGGAATCACGCACCCTGCTGCAGTAGCCTTGTGTAGGCAGCTGGTTTCTATGACTCCAGACGCTCTAGAATGCGTGTTTCTT |
| | | GCAGATTCTGGCTCAGTGGCCGTTGAAGTAGCAATGAAAATGGCTTTGCAGTACTGGTTGGGCCGAGGTGAAACCAGGCAACAATTCC |
| | | TGACGCTTAAGCGAGGGTATCATGGCGATACTTTTGCTGCTATGTCCGTTTGTGATCCTGACAACTCCATGCACAGTCTTTGGCGCGGG |
| | | TATTTACCGGAACACCATTTTGCAGCTGCACCTCAGTGTGGCTTCGATGACGAGTGGGATGCAAGGGACGCTGAGGATTTTATTAGGTT |
| | | GATTCAACAACATAGAGGTCAGTTAGCGGCTGTTATTCTCGAACCAATTGTACAAGGCGCAGGAGGTATGAGATTCTATCATCCAAGGT |
| | | ATCTGCAATGTGTTAGAGAGGCCTGTGACAGACATGGTATCCTGTTAATTGCCGACGAGATAGCTACCGGTTTCGGTAGAACTGGCACT |
| | | TTATTTGCTTGTGAGCACGCGGCAATTTCTCCAGACATTATGTGTGTCGGAAAGGCGCTTACCGGAGGAACTATGTCATTGGCCGCAAC |
| | | ACTGACGACTCGCGATGTGGCAGATACCATTTCATTAAGCCCGGCAGGTTGTTTTATGCATGGACCAACTTTTATGGGTAATCCACTTG |
| | | CGTGTGCCGTGGCCAGTGAATCCCTTGCCATGGTACAGGAGGGAGACTGGAGGCAACAGGTGGCAGCCATTGAGAGGCAACTGCGT |
| | | GGAGCCTTATTGCCTCTGAGACACGCTCCGGCTGTAGCAGATGTAAGAGTACTTGGTGCAATAGGGGTAGTGGAAACTCACCAACCTG |
| | | TAAATATGGCTGCCTTACAACAATTCTTCGTAAGTCAAGGCGTCTGGATTCGCCCGTTCGGCAGACTTATTTATCTTATGCCTCCTTATA |
| | | TTATATCACCTGAAGCTTTGGATAAGCTGGTTAATGCAATCGGAGCTGCACTAGAACAGCCCGCACATTTTTTATCATGA |
| 334 | Stenotrophomonas | ATGAGTGTCTCTTGTTGCTGCAGGAATCTTGGGAAGACGATCAAAAAAGCTATCCCCTCTCACCATCTGCATTTGAGGTCCTTAGGTGG |
| | maltophilia BioA nucleic | CTCTTTATATAGACGCATTCAATCTTCTTCAATGTTAGCTGATCCAACACCAAGCCCTTTGGCTCATCATTGGAGACAAAGGGACCTTCA |
| | acid with MTP (SEQ ID | GGTTCTATGGCATCCATGCACGCAGATGCGTGAACACCCTGACACTCTACCATTAGTGCCGATTGCACGTGGAGAGGGAGCTTGGCTC |
| | NO: 334) | ATCGATCATGATGGTAATCGTTATCTTGATGCTGTATCAAGCTGGTGGACTAACCTGTTTGGCCACGCAGAACCGCGAATTGGAGGCGC |
| | | AATTGCTGCTCAAGCCACTCAGCTTGAACAAGTAATGTTAGCTGGATTCGGCCACGAACCCGCTATAACTTTAGCTGAACGGTTGCTGG |
| | | CATTAGCTCCACGTCAGCCTGGCAGAGAGCCACTGGCCAAGGTGTTCTATGCAGATAATGGCTCAGCCGGCGTGGAAGTAGCTCTGAA |
| | | GATGGCATTTCAATATTTTCAGAACAGAGGCGAGTCCCGGCGCACACGATTCATTGCTCTGGAAAATGGCTACCACGGTGAAACCTTG |
| | | GGTGCCTTGGCACTTGGAGATATCCCACTGTATAGGAGAGTCTATGCACCCCTTCTTGCCGAGGCCCTTTTTGCACCTTCCCCGGATG |
| | | CATATCTTGCTGAACCTGGACAATCCGCTGCCGATAGAGCCAGGCAAGCAGCCGATGGCTTAGCTACTCTTTTCGATCAACACCCAGG |
| | | TGAAATCTGTGCAGTGATACTAGAGCCGCGCCTGCAATGTGCTGGGGGTATGAGAATGCACGATCCGGTGTATCTTCAGAGGGTGAGA |
| | | GAGTTATGCGATGCCCATGGTGCCTTTATGATTGCCGATGAGATAGCGACAGGTTTTGGCCGTACTGGAACGTTGTTCGCTTGCGAAC |
| | | AAGCCGGTGTTATGCCTGACCTTATGTGTCTGTCTAAGGGCCTCACAGGTGGATTTCTTCCTCTGGCCGCCGTCCTTGCAACTCAGGC |
| | | ACTCTATGATGCATTTCTTGATGATTCTAGAGAACGTGCATTTCTTCATAGTCACTCTTATACTGGTAATCCACTAGCCTGTGCAGCAGC |
| | | ACTTACCACACTAGATATTTTTAGAGATGATGATGTAATCGCAAGGAATCGGAGTATAGCCAGCGTGATGGGGACACTCGCCGCACCCT |
| | | TTAATGACCACCCCCACGTGGCCGATGTCAGGCAGGCAGGAATGGTCGTCGCCTTCGAATTGTCCCGTGATGGTAACAAGAGGACCC |
| | | CATTTGATCCTGCTATGAGATTAGGTTTACATGCATACAAAGCTGCCCTTAAGCGTGGTGTAGTTCTTCGTCCTCTCGGTGATGTCTTGT |
| | | ACTGGATGCCTCCTTACTGTGTAGACGACGAGCAGCTTGAGCTGCTCGCTCATACCACTCTTGCTGCTATAGATGAGGCAATTGCATGT |
| | | GCCTGA |
| 335 | Chroococcidiopsis sp. | ATGTCCGTGTCTTGTTGTTGTAGAAACTTAGGTAAGACCATAAAGAAAGCGATCCCGTCTCATCACTTACATCTTAGATCCTTAGGAGGC |
| | CCMEE 29 BioA nucleic | TCATTGTACAGAAGAATTCAATCATCATCTATGTCCGTGGAGGAAATTCATCCTAGCTATCCTATTTGGCACCCTTCCACTCAAATGAAAA |
| | acid with MTP (SEQ ID | CTTCTCCACCTCCATTGAAGGTAGTTAAGGGACAGGGCGTCATGCTTGAGCTCGAGGACGGTAGGCAGATACTGGACTGTATTTCCTC |
| | NO: 335) | ATGGTGGGTAACAATCCATGGTCATTCTCATCCTATCTTGGCAAATGCCTTATATCAACAGGCACAACAGCTTGAACACGTCATTTTTGC |
| | | AGGGTTTACTCATGAACCGGCGGAACAATTAGCAAGAAAGTTATTGAACCACTTGCCTAAATCTCTAACCCGTGTTTTTTTCAGTGACAA |
| | | TGGAAGTACTGCAGTTGAAGTGGCTCTGAAAATGGCATACCAATACTGGTATAACCAAGGTGAGACCCAGCGTACATCTTTCATCGGTT |
| | | TTGAGGGAGGTTATCATGGAGATACCCTGGGAGCAATGTCTGTCGGTGGTTCCACTAGTTGGTGGCAAACTTTTCAACTACTTATGTTT |
| | | TCAATCGATGTTGTTCCTTTCCCAGCAACTTTCGATCGTGACCCAAATGTTGAGGCACATGAGGCCCAGTCTCTAGAAACAATCACAAG |
| | | ATTATTGAAACAAAATCCCGATCGGTATGCCGGAATCTTCATCGAGCCATTGGTACAAGGTGCTGCGGGAATGCGTCTTTGTAGACCTC |
| | | AGTTTTTGCAGGAGCTTGAATCTTTAGCACGATCATTTGACGTTTTGCTTATATATGATGAAGTCATGACTGGTTTTGGTAGAACAGGGG |
| | | AGTTTTTTGCTTGTTTAAAGTCAGCTACAACTCCAGATATACTTTGCTTATCTAAGGGCTTGAGCGGGGGATGTTTGCCTTTGGCAGTGA |
| | | CTCTTGCTACCGAGGATATTTATCGAGCATTTTACTCTGATGATATCCATAAGGCATTCTTACATGGACATAGCTACACTGGTAACCCGT |
| | | TGGCTTGCGCCACCGGTGTTGCTTCACTTGAGCTTTTAGAGCAGAATCCAGAGTTTCGGGCAATTGAACATCAACATAGGTATTATCTC |
| | | GAAAAGTGGCTGATTGGCCATCCCAAAGTGGAGCAATTGAGGACTTGTGGCACAATTGCCGCGATGGAGGTCCGTACGGAGGACCAA |
| | | TCCAGTTACTTTAATGCAATTGGTCCGACACTTAGGTCAAGATTTTTGGAGGAAGGATTTCTCTTGCGACCATTAGGAAACACTATTTAC |
| | | ATTATGCCCCCTTACTGTATAACTCCTAACCAGCTTGAATCTATTTACCAGGCAATTCGGCGCGTGCTCGATACTCTCTAA |
| 336 | Bacillus subtilis BioA | ATGTCCGTGTCTTGTTGTTGTAGAAACTTAGGTAAGACCATAAAGAAAGCGATCCCGTCTCATCACTTACATCTTAGATCCTTAGGAGGC |
| | nucleic acid with MTP | TCATTGTACAGAAGAATTCAATCATCATCTATGAAACAAAGCATGATAGAAAAAAGTAAAAAACATCTCTGGCTTCCTTTCACTCAAATGA |
| | (SEQ ID NO: 336) | AAGATTATGATGAAGATCCATTGATCATTGAGTCCGGAAATGGTATTATGCTCAAAGACATTTCCGGTAAGGAGTATTATGACGGATTTT |
| | | CTAGTGTCTGGTTGAACGTACACGGTCATCGTAAAAAGGAGATGGACGAGGCCATACAAAAACAGCTTGAAAAGATTGCTCATTCTACA |
| | | TTGTTGGGCATGACAAACGTCCCAGCCACAGAGCTCGCAGAGATGTTAACCGGCATTACTCCAGAGAAATTAACCCGCGTCTTTTATTC |
| | | AGATTCAGGGGCCGAAGCAATGGAGATTGCATTGAAAATGGCTTTCCAGTATTGGAAAAACATAGGAAGACCTGAGAAGCAAACCTTTA |
| | | TCTCTATGCAAAACGGGTATCACGGTGATACTATAGGCGCAGTTTCAGTAGGTTCAATTGAATTGTTTCATCACGTTTACGGACCATTGA |
| | | TGTTTGAAAGTTATAAAGCACCAATTCCATACGTCTACCGTTCTGAGTCAGGGGATCCTGTTCAATGTCGTGATGAATGTCTTCATGCGC |
| | | TCGAACAATTATTAGCCAAACACCATGAGGAAATCGCTGCTCTCACCATTGAATCCATGGTTCAAGGAGCATCAGGTATGATAGTCATG |
| | | CCAGAAGGATTCTTGGCAGGCGTAAGAAAATTGTGTACTGCCTATGACGTGTTGATGATTGTCGATGAGGTTGCTACTGGTTTTGGTAG |
| | | AACAGGAAAGATGTTCGCTTGTGAACACGAGAATGTTCAGCCAGACCTGATGGCAGCTGGTAAGGGGATCACAGGTGGATACTTGCCT |
| | | ATAGCAGTGACATTCGCTACCGAGGCAATCTACGAAGCTTTCTATGATGATTATGATAAAATGAAAACTTTTTTCCACGGCCACTCATAC |
| | | ACGGGTAATCAGCTGGGTTGTGCTGCGGCGATAGAAAATTTACGACTTTTCGAGTCAGAGCGTATAGTTGAACAAGTTGCAGAGAAGTC |
| | | CAAAACAGCAGCAGAATTCTTACACGATTTAAAGCAACTTTCTCATGTGGGAGACGTACGTCAACTCGGTTTCATGTGTGGAATAGAATT |
| | | GGTTCAAGATAAGGGTACAAAGCAACCCTACCCTCCTGAGAAGCGAATGGGATACAAGGTTAGCTTGAAAATGAGGGAGTTGGGAATG |
| | | TTGACGCGTCCACTGGGGGACGTCATCGCTTTCCTTCCCCCTTTGGCTTCAACTACAGATCAGTTGCAAGCTATGATTTCTATTATGAAG |
| | | GAAGCAATCGCTGAAGTCACTGGTAGGGGATTTTAG |
| 337 | Streptomyces | ATGTCCGTGTCTTGTTGTTGTAGAAACTTAGGTAAGACCATAAAGAAAGCGATCCCGTCTCATCACTTACATCTTAGATCCTTAGGAGGC |
| | viridochromogenes BioA | TCATTGTACAGAAGAATTCAATCATCATCTATGCCTGACCTGAGTGTCCCTGAATTGTTGGCACTGGATCGGAGACATGTGTGGCATCC |
| | nucleic acid with MTP | TTATGGCCCAATGCCAGGCAGAATTGATCCACTGGTTGTGGAGTCTGCATCTGGTGTGAGATTGAAGCTTGCCGGTGGGTCTGGGGAG |
| | (SEQ ID NO: 337) | CTTGTTGATGGGATGAGCAGTTGGTGGTCTGCAATTCATGGGTATAACCACCCGGTTCTGAACGAGGCTGCCCATGAGCAACTGAGCA |
| | | GAATGTCACACGTTATGTTCGGTGGTTTGACACATGAACCGGCTGTGCGATTGGCAAAATTGCTAGTTGACATGTCACCTGATGGGTTA |
| | | GAGCATGTGTTTTTGGCTGACAGCGGTAGTGTTAGTGTTGAAGTAGCCGTTAAGATGTGCTTGCAATACTGGCGAAGCCTTGGTAGGC |
| | | CAGGTAAGCAGCGCTTGTTAACTTGGCGTGGGGGGTATCATGGTGATACGTGGCAGCCGATGTCAGTTTGCGATCCTGAGGGTGGGA |
| | | TGCATGAACTTTGGACCGGAGTCCTTCAAAGACAGGTCTTTGCAGACCCACCTCCAGCTGTGTACGATGAGAGCTATAGCGATCATTTG |
| | | CGTTCACTTATCGAAAGACATGCTCATGAGTTAGCAGCTGTAATTGTTGAACCAGTTGTTCAAGGCGCTGGAGGGATGCGTTTCCATTC |
| | | ACCTGCTTATCTTAGGGTTTTGAGGGAAGCATGTGATGCCCATGATGTGCTTCTCGTATTCGACGAGATTGCTACCGGTTTTGGACGTA |
| | | CTGGTGCTCTTTTTGCGGCTGACCATGCTGCTGTGACCCCAGATGTTATGTGTATGGGCAAAGCCCTTACAGGGGGATATTTAACACTG |
| | | GCTGCAACTGTCTGTACCTCAAGAGTTGCAGATGGTATATCTAGGGGTGAAGTACCAGTGTTAGCCCATGGACCTACTTTTATGGGCAA |
| | | CCCACTCGCTGCAGCTGTAGCTTGCGCTTCTATCGAGCTTTTGTTAGGTCAAGATTGGTTAGCTGAGGTAAAGAGAATTGAGGCCGGAT |
| | | TGCGTGAAGGTCTTGCTCCTGTTGCTGATCTTCCCGGAGTTAGGGATGTTCGCGTACTCGGTGCAATAGGGGTTGTCCAATTGGATCA |
| | | CGCTGTAGATATGAAGGCCGCTACTGACGCAGCTGTTCGTGAGGGCGTGTGGCTACGTCCCTTTCGCGATTTGATTTATACAATGCCA |
| | | CCTTATGTGACAGGCGATGCTGATCTCGCCAGGATTGCAAGGGCTGTCTGTGCTGCTGCTAGAGAGGGATGA |
| 338 | Pedobacter hartonius | ATGTCCGTGTCTTGTTGTTGTAGAAACTTAGGTAAGACCATAAAGAAAGCGATCCCGTCTCATCACTTACATCTTAGATCCTTAGGAGGC |
| | BioA nucleic acid with | TCATTGTACAGAAGAATTCAATCATCATCTATGACATTAACACAACGAGATGCAGAGTCAATTTGGCACCCTTATACTCAACAGTTAAATG |
| | MTP (SEQ ID NO: 338) | CTAGGCCTCCTTTACCTGTGTTGTCTGGCAAGGGCGCTTACTTATACGATGAGGAGGGTTGTAGATATATTGACGCAGTTAGCAGTTGG |
| | | TGGGTTACTTTGCATGGTCATTCCCATCCATACATTGTTGAACGCGTTACTGCTCAACTTCAACAATTGGACCAGGTTATCTTCGCTGGC |
| | | TTTACTCATGAACCAGCGATTGCTTTGGCGGAGAATTTGTTGGCTATCCTTCCCGAGGGACAAAAAAAAGTCTTCTACACAGATAACGG |
| | | AAGCACAGCTGTCGAAGTAGCTCTTAAGATGTGTGTGCAGTACTGGTTTAATAAGGGAAAATCCAGGAAAAAGGTTTTGGCCTTCAACA |
| | | ATGGATACCATGGAGATACTTTTGGAGCAATGTCCGTTTCTGGTCGAAGCGCCTGGACCGCTCCATTCGATAATTTGCTCTTTGAGGTA |
| | | ATTTTTATTGACACTCCTACTGAGGAGAACCTTCCGGTTCTGAAGGAAATTATAAGCGTGCATGCTGCCGAACTTGCATGCTTTGTTTAT |
| | | GAACCTCTCGTCCAAGGTAGTGCCGGAATGTTGATGTATGAAGCTCCTGCATTGGATCAGCTGATGGCTCATTGCCTCGAAGAGGGTG |
| | | TTTTAATGATCCAGGACGAGGTGTTTACTGGATTCGGCAGAACCGGAAAGAACTTCGCGGCTGATCACCTCAAAACCCAGCCTGATGTT |
| | | ATGTGCTTTTCTAAAGGTCTGACTGGTGGCACTATGCCCCTCGGCGTGACCACTTGCACTGAAGATATTTATAATGCATTTCTGAGTGAA |
| | | GATAAGTTAAAGACTCTTTTCCATGGACATTCATTCACAGCGAACCCTCTAGCTTGTACCGCTGCCTTAGCATCAATTGAGTTATTGTTG |
| | | CAACATGAAGCTCAAGATAACATTCGAAGAATTTGCAACAGGCACGCTGAGTTCTCATTGAAAATTTGCGGTCACCAAAAAGTCGCTGC |
| | | AGTGAGACAAACTGGTACCATTATTGCAATCGAGTGGCAAACTGAGTCTGGAACAAGTTATTTCAGTAAACTTAGGAATCTGTTGTACGA |
| | | TTATTTTATGGATAAGGGTATACTAATGAGACCACTAGGTAACGTTATATATATACTTCCGCCTTATTGCATTAGCGATGAGGACCTAAAA |
| | | AAAATATACCAAGAAATTCAAAATGCGTTGGAAATCTTTTGA |
| 339 | Chitinophaga filiformis | ATGTCCGTGTCTTGTTGTTGTAGAAACTTAGGTAAGACCATAAAGAAAGCGATCCCGTCTCATCACTTACATCTTAGATCCTTAGGAGGC |
| | BioA nucleic acid with | TCATTGTACAGAAGAATTCAATCATCATCTATGCTATGGTATCCATACACTCAGATGAGACAAATTAATGAATTACCTAAGATGGTGAGTG |
| | MTP (SEQ ID NO: 339) | GCGAAGGCGTTATGATGCACCTTGAGGACGGAAGGAGTCTAATAGATGGCATTTCTAGCTGGTGGGCTGTTATCCATGGCTATAACCA |
| | | TCCTGCTCTTAATGCTGCTTTATTAACGCAGGCTAACAAGTTCGCCCATGTTATGCTTGGTGGAATGACTCATAATCCAGCTCTAGATTT |
| | | AGCTGCCAAACTTGTTAGTATCACTCCACAGGGGCTGAATCATGTATTCTTCAGCGATTCAGGTTCAATAGGAGTCGAAGTCGCATTAA |
| | | AGATGTCTATTCAATACTGGAAAAATATTGGGTACAATGGAAAATCTAAGATAATTTCACTTAGAAACGGGTATCATGGCGACACATTTAA |
| | | GGCCATGGAAGTATCTGACGATTCTGATTTTACTAGGGCTTTCTCTGATGTTTTACGTAGGGGATACATCCTTGATATCCCGGAGGGCG |
| | | GGTTCGACGCAGATGCAGCGCTAGTAAAACAGGCTACTGACAAACTGGAGGCTTTGTTACGACGCGAGCACCACAATATTGCCGCTTT |
| | | TATAGTTGAACCAATAGTTCAATGCGCGGGTGGGTTCAATATATACTCACCGCTTTACTTGAAGGCTGCCCGAGAATTGTGTACTAAGTA |
| | | TAATGTACTTTTTGTCTTTGATGAGGTTGCCACAGGGTTTGGTAGGACAGGAAAGCTTTTTGCTGCTGAACACGCTGGAGTTACACCAG |
| | | ATATTATGATCCTCGGCAAAGCGCTAACGGCTGGCTACATGGGACACGCTGCCACCTTAGCTACATCAGCTGTTTTTGATAGTTTCTTG |
| | | GGAGACAATTATGAAAAAGCACTTATGCACGGCCCTACTTTTATGGCAAATCCCTTGGCCTGCGCTGTCGCTTTGAGATCAATATCAATT |
| | | ATTGAAGACGAACAATATCTTCAAAAGATCGCTAAAATACAATCAATTATAAGAGAGCAATTTGATACTTTCTCCTCACCCGCAATTGTGG |
| | | CGAAGAGGAGTATAGGCGCTATCGGGGCGTTGGAAATGAAAGATGCAGCTTGTCTTTCTGGCTTCAAAGAGTTTAGTCAACAGAGAGG |
| | | AGTATGGTTGAGACCCATTGGTAATGTGTTATATTTAATGCCACCTTATATTATCTCAGAGAAGGAGCTTTTGACAATATTACAGGTAATA |
| | | AAGGATTGGATACAGCAGATTAAATAA |
| 340 | Tenacibaculum | ATGTCCGTGTCTTGTTGTTGTAGAAACTTAGGTAAGACCATAAAGAAAGCGATCCCGTCTCATCACTTACATCTTAGATCCTTAGGAGGC |
| | adriaticum BioA nucleic | TCATTGTACAGAAGAATTCAATCATCATCTATGAACCTTACCGAGCGAGATAAGAAGCATTTGTGGCATCCATTGACTCAACACCAACTT |
| | acid with MTP (SEQ ID | TATCCTAATCATCTTGCCATTACCAAGGCGAAAGGAGCAATTTTATACGATGATAAAAATAATGAATACATCGATGGTATCGCTAGTTGG |
| | NO: 340) | TACACTTGTATGTACGGCCACTGTAATGAGTATATTACTAGCTCAGTCTATAAGCAGATGCAGCAATTAGACCATGTTGTGTTTGCAGGA |
| | | TTTACCCACGAACCAGCAATCAAGCTGAGTGAAGAATTGATAAAGATCTTGCCCGAGAACCAAGAGAAAGTTTTTTTCTCAGATAACGGT |
| | | AGTACTAGTGTAGATGTCGCTATTAAAATGGCACTCCAATACCATTTTAACCAGGGGGAAAAGAAAGGGAAGATTATTGCCCTAGAAGA |
| | | TGGTTTTCATGGAGACACATTTGGTGCGATGTCAGTATCTGGTCTTTCAGTCTATAATGGCCCATTTGAAGATTTTTTCATAGATGTCGCT |
| | | AGGATCCCCGTTCCCACTCAAGAGAACTTTGAACATGTGAAAGAAGAATTCATAAAGCTTATTACCGAGCACAACGTAGCCGCATTTGTT |
| | | TACGAACCATTGGTTCAAGGAGCCGCAGCTATGAAAATGTTTGATGGTAATTTGTTAAACGAGCTTATTAAAATTGCTCAAGAGAACAAT |
| | | GTGCTAGCTATTGCTGACGAGGTGATGACCGGCTTTGGAAAAACAGGGAAAAACTTTGCTTCAGAATACCTCGAGCATAAGCCAGACAT |
| | | AATTTGTCTTTCAAAGAGTTTGACAGCAGGATTAGTTCCAATGGCTATCACATCATGTAGCCAGAAGGTTTACAATGCATTCCTTTCAGAT |
| | | GATATCGGGAAGGGTTTCTTCCATGGCCATACTTATAGTGCTAATCCGGTAGCATGTAGCGCAGCTCTCGCAGGAATTGAGTTGCTACA |
| | | AAGTGAAGAGATCCAAGGGAATATACAAAGGATTATCAAATCTCACCAAGAGTTTGATTCCAAAATTAAGAAACATCCTAAAGTAAAGTC |
| | | TACAAGACAAAAAGGTATTATTTATGCTCTCGATCTTAATGTAGAAATGGAAAGATACGGAGACCTAAGGTATAAGCTATTCAATTTTTTT |
| | | ATGGAAAACGGAGTCTGCCTGAGGCCTTTAGGAAACACAATATATATTTTAGCACCATTCGTTATTACAAATGAGCAATTAGAAAAGATC |
| | | TACAAAACAATCGAGAAAGCACTCGAAATTCTATGA |
| 341 | Ostreococcus tauri | ATGAGTGCTCCCATCGCGAGGAGGGCATCATCTGTTGCCCGAGGAAGGACCCGATGGTTGACATCTACTAGCATTGAAAGATCCAGGG |
| | BioDA nucleic acid | AATGGTTTGTTAGAAGCCTCGGCAGTGCCTCTGATGGAGCTGCTTCTGGTAACGGACGGGCACTAGAGCTGAGTATAGCGTCCTTTGC |
| | (SEQ ID NO: 341) | TATTTGGGGAGCAAACACTGGAGTAGGAAAAACTCTCGCAAGCGCTGGGTTAGCTAGAGCCGCCACCCGGCGGGGCACTGCTACTAC |
| | | ATTCCTCAAGCCCGTTCAAACTGGTTATCCGGAGGATTCTGATGCTGGTTTTGTTGCTAGACAGACAAAAGGTAATGAAACCGCCGGAG |
| | | AGCATGCTGCCGTGGCAAGTGGCTCTACTGAAGGTGGGGGAGAAGGATTTGCCGCTGGTGTGACAAGGGCCCACACGGAGTTTGCCT |
| | | GGCGTCGGGCTGCCGGGCCTCATCTCACAGCTGCAGAAGAAGGGCGTCCAGTCTCTGATGAGGAAATTGTTAGGGCAACTAGGAATT |
| | | ACTTGAGAGAGTTCGCTGAATCAGCTCGGGAAGGTAACGCTAGAGGTTTTGCCGTTATCGAGACCGCTGGAGGAGTAGCATCTCCAGG |
| | | ACCATCAGGTACCTTGCAGTGTGAAATTCTAAGGGCATTAAGATTACCCGCAATTTTAATAGGTGATGGCCGACTTGGTGGGATTTCTA |
| | | CAACAACGTCTGCATATGAATCACTCCACGCGAGAGGATACGATGTTGTGGGTGTTTTGATGGCTGATGACGGATATGCTAACCATGAA |
| | | GCAGTTGCTAAGATTTTACCTAGCGGAACAAAGGTATTTGCCTTGCCTCCATTACCTCCGCAAGGTAAAGCCGACGCATGGTTGGAAAA |
| | | ATCAGCTGGCGTGTTTAGTGAGGCTTTCGAAACTGCCATGAACTGGCATGAGAAGAGAATACAGGCATTACAGAAATTACCAGGTGAAG |
| | | CTCTTGAGAAAATTTGGTGGCCATTTACACAACATGCAACTGTTGAACCGGATACTGTAACTGTGATTGATGGTCGTTATGGTGAGGACT |
| | | TTGAGATTTATGACCAAGAGAAAGGCCGTGATGGGGCTATAGACTTGCGATTCGACGGCGCCGCTTCCTGGTGGACCCAGGGGGTCT |
| | | CCGCAGAGTTACAACAGGAACTCAATTCAGCTGCAGCCAATGCAGCTGGTCGTTACGGGCATGTTATGTTTCCCGAAAATGTGCACCAA |
| | | CCTTCCATGGATGCTACTAACGCCCTGCTTGAGGGTCCTGGCAAAGGGTGGGGTTCTAGAGTGTTTTACTCCGATAATGGCAGCACAG |
| | | CTACCGAAGTAGGATTGAAAATGGCTTTTAGGAAGTATTACGTGGACGCAGGGCTCGTGAGCAAAGACGGTCACCAAAGAGCAAAGGA |
| | | TCTTATTAACGACGGATCATGTCACCTTCCACAATTGCGGATCATTGCTCTTGACGGTTCCTACCATGGCGATACTTTGGGGGCAATGG |
| | | ATATGCAGTCACCTTCAGTTTTCACAGGTCCTATGCAAACCCCATGGTATCAACCACGGGGCTTATTTCTTCAGCCGCCTAGCCTAGCA |
| | | ATACGGAATGGAGAGTGGACTGTTGCTCTACCCCCCCATGGGCTACTAAATGATGGAGAGGACCGCTTATTCCACGATGGGAAGGATC |
| | | TCGAGTCCAATACGACTTGGAAATCTAAAGCAGATGCATTTGATATCGGGAAGAGGTTGGACTCACCTTTAGCAAACGAGTATAGGCAG |
| | | GCAGTCGATAAAGCCTTGGAAAGAGCAGAACAGGATGCTAAGGCCGGACATATGGGACCTGTGGGCGCACTAATGATGGAGCCTGTA |
| | | CTCCACGGCGCTGGCGGAATGGTACTAATTGATCCTTTGTTCCAGAACATTTTGATGAAGGTGTGTAGGGAACGTAAGATCCCAATCGT |
| | | GTTGGATGAGGTATTTGCTGGCATTTGGCGCTTGGGCACTGAAGGTGCGTGGGAGATTATCGGAGAAAAGCCCGATATATCTTGCTAT |
| | | GCAAAGCTACTGACGGGAGGCTTGATGCCCATGTCTGCAACCGTCGCTACAGCCGACGTTTTCGATGTGTTCTATGGTCCGGGTAAAG |
| | | CGCAGGGTCTGCTTCACGGGCATTCCTATACTGCATATCCTATCGGATGCGCTGTTGCTAGCAAGGCATTGTCCCTTTATAAGGATCCA |
| | | GCGAAGAATCCGAATCTGATAGTCGATGCTTCATCTAAAGGAGATACACCTACTGCATACTTGAGAGAATTATGGGATGAGTCCAAATT |
| | | GAAGGAGATATCAAACAAATCCAGTGTAAGACGTGTTTTCGGTCAGGGTTGCGTTTTAGCCGTGGAATTGGAGGTTGAAGGCGAAGGC |
| | | GGCTATTCATCAAATGCAGCTAAAGAAGTTGTTCTACGATTGAGACCCCATTCAGTTCAAGCAAGGCCTCTGGGTAACGTTGTTTATCTT |
| | | ATGTGCGCACCTACTACACCTAAGGCTACTTGCGATCGCTTATTGAACGTCCTTGCTGAGGAGTTATCAGAATAA |
| 342 | Arabidopsis thaliana | ATGATTCCCGTGACCGCCACTCTTATTCGTCACAGGTTGAGGCACTTGCGTCATCGAATTAGGTTTAAATCAACCTCTGTATCACCATTC |
| | BioDA nucleic acid | CATTTGCCGCTCAATCATCCTACTTATTTGATTTGGTCTGCCAACACTAGCCTGGGCAAAACTTTGGTCAGTACGGGGATAGCAGCCAG |
| | C388D (SEQ ID NO: 342) | TTTTCTCTTACAACAACCTAGTTCATCAGCAACAAAACTATTGTATCTCAAACCCATCCAGACAGGTTTTCCTAGTGATTCTGACTCAAGA |
| | | TTTGTTTTTTCAAAGCTCGACTCATTGTCATTGCGAAGACAAATTCCTATAAGTATCTCAAATTCCGTACTTCATTCATCACTCCCTGCTG |
| | | CTAAGTCTCTTGGCCTAAATGTTGAAGTAAGCGAGTCTGGAATGTGTAGTCTCAATTTTCGGGATGAGAAAACTGTTACCGGCGCTCCA |
| | | GAGTTGTTGTGCAAGACACTTTATGCTTGGGAGGCTGCAATTTCACCCCATCTTGCTGCAGAGAGAGAAAACGCAACTGTTGAAGACTC |
| | | TGTGGTTCTTCAAATGATTGAGAAGTGCCTGAAGGAGGAGATGGAGTGCGGAGTTAAATCTGAGAAGAGTGATCTCTTGTGTCTAGTCG |
| | | AAACTGCTGGCGGAGTTGCTTCTCCTGGTCCATCTGGAACCTTGCAATGTGACCTGTATAGGCCGTTTAGGCTCCCCGGAATTCTAGTT |
| | | GGAGACGGCAGGTTGGGGGGTATCTCTGGCACAATAGCAGCCTATGAGAGTCTCAAGCTTCGTGGATACGATATCGCTGCTGTTGTCT |
| | | TCGAAGATCACGGACTAGTTAACGAAGTGCCACTTACATCATATTTGAGGAATAAGGTACCTGTCCTCGTATTACCTCCTGTCCCTAAAG |
| | | ACCCTTCAGACGACTTGATCGAATGGTTCGTGGAGTCCGATGGCGTATTCAAAGCACTTAAGGAGACGATGGTATTAGCGAACCTTGAA |
| | | CGTCTTGAAAGGTTGAATGGTATGGCCAAACTGGCTGGTGAAGTGTTTTGGTGGCCATTCACACAGCATAAGCTTGTTCACCAAGAGAC |
| | | CGTAACTGTGATTGACTCCAGATGTGGCGAGAATTTTTCTATTTATAAGGCATCAGATAATTCTTCTTTGAGTCAACAGTTCGATGCTGAT |
| | | GCAAGTTGGTGGACACAGGGGCCTGACCCTACTTTTCAGGCTGAATTGGCTCGCGAGATGGGATACACAGCGGCCAGGTTCGGCCAT |
| | | GTAATGTTCCCCGAAAATGTTTATGAACCTGCTCTTAAATGTGCTGAGCTTTTGTTAGACGGTGTAGGAAAGGGATGGGCTTCACGCGT |
| | | ATACTTCTCTGATAATGGCTCAACCGCAATAGAGATCGCTTTGAAGATGGCTTTTAGGAAATTTTGCGTTGACCACAACTTTTGCGAAGC |
| | | CACAGAGGAAGAGAAACACATTGTGGTTAAGGTAATTGCCCTGAGAGGCTCATACCACGGCGATACTTTGGGTGCTATGGAGGCTCAG |
| | | GCGCCATCACCATATACTGGTTTTCTTCAGCAACCCTGGTACACGGGCCGGGGCCTATTTTTGGATCCTCCAACCGTTTTTTTGTCTAA |
| | | CGGTTCTTGGAACATATCTTTGCCGGAAAGTTTCTCAGAAATTGCTCCTGAGTACGGCACCTTCACAAGTCGTGATGAAATCTTCGATAA |
| | | GTCAAGAGACGCTTCAACACTTGCAAGAATCTATTCAGCTTATCTCTCAAAGCATTTGCAGGAGCATTCAGGAGTTAGGCAGAGTGCCC |
| | | ATGTTGGTGCATTAATCATTGAGCCGGTGATTCATGGTGCAGGAGGAATGCATATGGTGGATCCTCTATTTCAGAGAGTCCTCGTTAAT |
| | | GAATGTAGAAATCGTAAGATACCTGTCATATTTGACGAAGTCTTCACTGGTTTTTGGAGATTGGGAGTGGAAACGACAACAGAACTTCTA |
| | | GGATGCAAACCTGATATTGCGTGCTTTGCTAAGCTATTGACGGGCGGTATGGTACCCTTGGCTGTGACATTGGCTACAGATGCAGTTTT |
| | | CGATAGTTTTAGTGGTGATAGCAAATTGAAAGCTCTATTGCACGGACATAGTTACTCTGCTCACGCCATGGGATGCGCTACCGCGGCGA |
| | | AGGCAATCCAATGGTTTAAAGATCCTGAAACCAATCACAATATCACATCTCAAGGCAAGACCCTCAGGGAACTGTGGGACGAAGAACTA |
| | | GTTCAGCAAATCTCTTCACACTCAGCAGTGCAACGCGTTGTTGTCATAGGAACCTTGTTTGCTCTCGAGTTGAAGGCAGACGCAAGCAA |
| | | CTCAGGGTACGCTTCACTCTATGCTAAATCTTTATTGATTATGTTGAGGGAAGATGGCATCTTTACACGACCTCTGGGTAACGTGATCTA |
| | | CCTCATGTGCGGTCCTTGTACGAGTCCCGAAATATGTAGACGACTGCTGACTAAACTGTATAAGCGTTTGGGTGAATTTAATCGTACTTA |
| | | A |
| 343 | Arabidopsis thaliana | ATGATTCCCGTGACCGCCACTCTTATTCGTCACAGGTTGAGGCACTTGCGTCATCGAATTAGGTTTAAATCAACCTCTGTATCACCATTC |
| | BioDA nucleic acid | CATTTGCCGCTCAATCATCCTACTTATTTGATTTGGTCTGCCAACACTAGCCTGGGCAAAACTTTGGTCAGTACGGGGATAGCAGCCAG |
| | W392S (SEQ ID | TTTTCTCTTACAACAACCTAGTTCATCAGCAACAAAACTATTGTATCTCAAACCCATCCAGACAGGTTTTCCTAGTGATTCTGACTCAAGA |
| | NO: 343) | TTTGTTTTTTCAAAGCTCGACTCATTGTCATTGCGAAGACAAATTCCTATAAGTATCTCAAATTCCGTACTTCATTCATCACTCCCTGCTG |
| | | CTAAGTCTCTTGGCCTAAATGTTGAAGTAAGCGAGTCTGGAATGTGTAGTCTCAATTTTCGGGATGAGAAAACTGTTACCGGCGCTCCA |
| | | GAGTTGTTGTGCAAGACACTTTATGCTTGGGAGGCTGCAATTTCACCCCATCTTGCTGCAGAGAGAGAAAACGCAACTGTTGAAGACTC |
| | | TGTGGTTCTTCAAATGATTGAGAAGTGCCTGAAGGAGGAGATGGAGTGCGGAGTTAAATCTGAGAAGAGTGATCTCTTGTGTCTAGTCG |
| | | AAACTGCTGGCGGAGTTGCTTCTCCTGGTCCATCTGGAACCTTGCAATGTGACCTGTATAGGCCGTTTAGGCTCCCCGGAATTCTAGTT |
| | | GGAGACGGCAGGTTGGGGGGTATCTCTGGCACAATAGCAGCCTATGAGAGTCTCAAGCTTCGTGGATACGATATCGCTGCTGTTGTCT |
| | | TCGAAGATCACGGACTAGTTAACGAAGTGCCACTTACATCATATTTGAGGAATAAGGTACCTGTCCTCGTATTACCTCCTGTCCCTAAAG |
| | | ACCCTTCAGACGACTTGATCGAATGGTTCGTGGAGTCCGATGGCGTATTCAAAGCACTTAAGGAGACGATGGTATTAGCGAACCTTGAA |
| | | CGTCTTGAAAGGTTGAATGGTATGGCCAAACTGGCTGGTGAAGTGTTTTGGTGGCCATTCACACAGCATAAGCTTGTTCACCAAGAGAC |
| | | CGTAACTGTGATTGACTCCAGATGTGGCGAGAATTTTTCTATTTATAAGGCATCAGATAATTCTTCTTTGAGTCAACAGTTCGATGCTTGC |
| | | GCAAGTTGGTCTACACAGGGGCCTGACCCTACTTTTCAGGCTGAATTGGCTCGCGAGATGGGATACACAGCGGCCAGGTTCGGCCAT |
| | | GTAATGTTCCCCGAAAATGTTTATGAACCTGCTCTTAAATGTGCTGAGCTTTTGTTAGACGGTGTAGGAAAGGGATGGGCTTCACGCGT |
| | | ATACTTCTCTGATAATGGCTCAACCGCAATAGAGATCGCTTTGAAGATGGCTTTTAGGAAATTTTGCGTTGACCACAACTTTTGCGAAGC |
| | | CACAGAGGAAGAGAAACACATTGTGGTTAAGGTAATTGCCCTGAGAGGCTCATACCACGGCGATACTTTGGGTGCTATGGAGGCTCAG |
| | | GCGCCATCACCATATACTGGTTTTCTTCAGCAACCCTGGTACACGGGCCGGGGCCTATTTTTGGATCCTCCAACCGTTTTTTTGTCTAA |
| | | CGGTTCTTGGAACATATCTTTGCCGGAAAGTTTCTCAGAAATTGCTCCTGAGTACGGCACCTTCACAAGTCGTGATGAAATCTTCGATAA |
| | | GTCAAGAGACGCTTCAACACTTGCAAGAATCTATTCAGCTTATCTCTCAAAGCATTTGCAGGAGCATTCAGGAGTTAGGCAGAGTGCCC |
| | | ATGTTGGTGCATTAATCATTGAGCCGGTGATTCATGGTGCAGGAGGAATGCATATGGTGGATCCTCTATTTCAGAGAGTCCTCGTTAAT |
| | | GAATGTAGAAATCGTAAGATACCTGTCATATTTGACGAAGTCTTCACTGGTTTTTGGAGATTGGGAGTGGAAACGACAACAGAACTTCTA |
| | | GGATGCAAACCTGATATTGCGTGCTTTGCTAAGCTATTGACGGGCGGTATGGTACCCTTGGCTGTGACATTGGCTACAGATGCAGTTTT |
| | | CGATAGTTTTAGTGGTGATAGCAAATTGAAAGCTCTATTGCACGGACATAGTTACTCTGCTCACGCCATGGGATGCGCTACCGCGGCGA |
| | | AGGCAATCCAATGGTTTAAAGATCCTGAAACCAATCACAATATCACATCTCAAGGCAAGACCCTCAGGGAACTGTGGGACGAAGAACTA |
| | | GTTCAGCAAATCTCTTCACACTCAGCAGTGCAACGCGTTGTTGTCATAGGAACCTTGTTTGCTCTCGAGTTGAAGGCAGACGCAAGCAA |
| | | CTCAGGGTACGCTTCACTCTATGCTAAATCTTTATTGATTATGTTGAGGGAAGATGGCATCTTTACACGACCTCTGGGTAACGTGATCTA |
| | | CCTCATGTGCGGTCCTTGTACGAGTCCCGAAATATGTAGACGACTGCTGACTAAACTGTATAAGCGTTTGGGTGAATTTAATCGTACTTA |
| | | A |
| 344 | Arabidopsis thaliana | ATGATTCCCGTGACCGCCACTCTTATTCGTCACAGGTTGAGGCACTTGCGTCATCGAATTAGGTTTAAATCAACCTCTGTATCACCATTC |
| | BioDA nucleic acid | CATTTGCCGCTCAATCATCCTACTTATTTGATTTGGTCTGCCAACACTAGCCTGGGCAAAACTTTGGTCAGTACGGGGATAGCAGCCAG |
| | F348C (SEQ ID NO: 344) | TTTTCTCTTACAACAACCTAGTTCATCAGCAACAAAACTATTGTATCTCAAACCCATCCAGACAGGTTTTCCTAGTGATTCTGACTCAAGA |
| | | TTTGTTTTTTCAAAGCTCGACTCATTGTCATTGCGAAGACAAATTCCTATAAGTATCTCAAATTCCGTACTTCATTCATCACTCCCTGCTG |
| | | CTAAGTCTCTTGGCCTAAATGTTGAAGTAAGCGAGTCTGGAATGTGTAGTCTCAATTTTCGGGATGAGAAAACTGTTACCGGCGCTCCA |
| | | GAGTTGTTGTGCAAGACACTTTATGCTTGGGAGGCTGCAATTTCACCCCATCTTGCTGCAGAGAGAGAAAACGCAACTGTTGAAGACTC |
| | | TGTGGTTCTTCAAATGATTGAGAAGTGCCTGAAGGAGGAGATGGAGTGCGGAGTTAAATCTGAGAAGAGTGATCTCTTGTGTCTAGTCG |
| | | AAACTGCTGGCGGAGTTGCTTCTCCTGGTCCATCTGGAACCTTGCAATGTGACCTGTATAGGCCGTTTAGGCTCCCCGGAATTCTAGTT |
| | | GGAGACGGCAGGTTGGGGGGTATCTCTGGCACAATAGCAGCCTATGAGAGTCTCAAGCTTCGTGGATACGATATCGCTGCTGTTGTCT |
| | | TCGAAGATCACGGACTAGTTAACGAAGTGCCACTTACATCATATTTGAGGAATAAGGTACCTGTCCTCGTATTACCTCCTGTCCCTAAAG |
| | | ACCCTTCAGACGACTTGATCGAATGGTTCGTGGAGTCCGATGGCGTATTCAAAGCACTTAAGGAGACGATGGTATTAGCGAACCTTGAA |
| | | CGTCTTGAAAGGTTGAATGGTATGGCCAAACTGGCTGGTGAAGTGTTTTGGTGGCCATGTACACAGCATAAGCTTGTTCACCAAGAGAC |
| | | CGTAACTGTGATTGACTCCAGATGTGGCGAGAATTTTTCTATTTATAAGGCATCAGATAATTCTTCTTTGAGTCAACAGTTCGATGCTTGC |
| | | GCAAGTTGGTGGACACAGGGGCCTGACCCTACTTTTCAGGCTGAATTGGCTCGCGAGATGGGATACACAGCGGCCAGGTTCGGCCAT |
| | | GTAATGTTCCCCGAAAATGTTTATGAACCTGCTCTTAAATGTGCTGAGCTTTTGTTAGACGGTGTAGGAAAGGGATGGGCTTCACGCGT |
| | | ATACTTCTCTGATAATGGCTCAACCGCAATAGAGATCGCTTTGAAGATGGCTTTTAGGAAATTTTGCGTTGACCACAACTTTTGCGAAGC |
| | | CACAGAGGAAGAGAAACACATTGTGGTTAAGGTAATTGCCCTGAGAGGCTCATACCACGGCGATACTTTGGGTGCTATGGAGGCTCAG |
| | | GCGCCATCACCATATACTGGTTTTCTTCAGCAACCCTGGTACACGGGCCGGGGCCTATTTTTGGATCCTCCAACCGTTTTTTTGTCTAA |
| | | CGGTTCTTGGAACATATCTTTGCCGGAAAGTTTCTCAGAAATTGCTCCTGAGTACGGCACCTTCACAAGTCGTGATGAAATCTTCGATAA |
| | | GTCAAGAGACGCTTCAACACTTGCAAGAATCTATTCAGCTTATCTCTCAAAGCATTTGCAGGAGCATTCAGGAGTTAGGCAGAGTGCCC |
| | | ATGTTGGTGCATTAATCATTGAGCCGGTGATTCATGGTGCAGGAGGAATGCATATGGTGGATCCTCTATTTCAGAGAGTCCTCGTTAAT |
| | | GAATGTAGAAATCGTAAGATACCTGTCATATTTGACGAAGTCTTCACTGGTTTTTGGAGATTGGGAGTGGAAACGACAACAGAACTTCTA |
| | | GGATGCAAACCTGATATTGCGTGCTTTGCTAAGCTATTGACGGGCGGTATGGTACCCTTGGCTGTGACATTGGCTACAGATGCAGTTTT |
| | | CGATAGTTTTAGTGGTGATAGCAAATTGAAAGCTCTATTGCACGGACATAGTTACTCTGCTCACGCCATGGGATGCGCTACCGCGGCGA |
| | | AGGCAATCCAATGGTTTAAAGATCCTGAAACCAATCACAATATCACATCTCAAGGCAAGACCCTCAGGGAACTGTGGGACGAAGAACTA |
| | | GTTCAGCAAATCTCTTCACACTCAGCAGTGCAACGCGTTGTTGTCATAGGAACCTTGTTTGCTCTCGAGTTGAAGGCAGACGCAAGCAA |
| | | CTCAGGGTACGCTTCACTCTATGCTAAATCTTTATTGATTATGTTGAGGGAAGATGGCATCTTTACACGACCTCTGGGTAACGTGATCTA |
| | | CCTCATGTGCGGTCCTTGTACGAGTCCCGAAATATGTAGACGACTGCTGACTAAACTGTATAAGCGTTTGGGTGAATTTAATCGTACTTA |
| | | A |
| 345 | Arabidopsis thaliana | ATGATTCCCGTGACCGCCACTCTTATTCGTCACAGGTTGAGGCACTTGCGTCATCGAATTAGGTTTAAATCAACCTCTGTATCACCATTC |
| | BioDA nucleic acid | CATTTGCCGCTCAATCATCCTACTTATTTGATTTGGTCTGCCAACACTAGCCTGGGCAAAACTTTGGTCAGTACGGGGATAGCAGCCAG |
| | G700S (SEQ ID NO: 345) | TTTTCTCTTACAACAACCTAGTTCATCAGCAACAAAACTATTGTATCTCAAACCCATCCAGACAGGTTTTCCTAGTGATTCTGACTCAAGA |
| | | TTTGTTTTTTCAAAGCTCGACTCATTGTCATTGCGAAGACAAATTCCTATAAGTATCTCAAATTCCGTACTTCATTCATCACTCCCTGCTG |
| | | CTAAGTCTCTTGGCCTAAATGTTGAAGTAAGCGAGTCTGGAATGTGTAGTCTCAATTTTCGGGATGAGAAAACTGTTACCGGCGCTCCA |
| | | GAGTTGTTGTGCAAGACACTTTATGCTTGGGAGGCTGCAATTTCACCCCATCTTGCTGCAGAGAGAGAAAACGCAACTGTTGAAGACTC |
| | | TGTGGTTCTTCAAATGATTGAGAAGTGCCTGAAGGAGGAGATGGAGTGCGGAGTTAAATCTGAGAAGAGTGATCTCTTGTGTCTAGTCG |
| | | AAACTGCTGGCGGAGTTGCTTCTCCTGGTCCATCTGGAACCTTGCAATGTGACCTGTATAGGCCGTTTAGGCTCCCCGGAATTCTAGTT |
| | | GGAGACGGCAGGTTGGGGGGTATCTCTGGCACAATAGCAGCCTATGAGAGTCTCAAGCTTCGTGGATACGATATCGCTGCTGTTGTCT |
| | | TCGAAGATCACGGACTAGTTAACGAAGTGCCACTTACATCATATTTGAGGAATAAGGTACCTGTCCTCGTATTACCTCCTGTCCCTAAAG |
| | | ACCCTTCAGACGACTTGATCGAATGGTTCGTGGAGTCCGATGGCGTATTCAAAGCACTTAAGGAGACGATGGTATTAGCGAACCTTGAA |
| | | CGTCTTGAAAGGTTGAATGGTATGGCCAAACTGGCTGGTGAAGTGTTTTGGTGGCCATTCACACAGCATAAGCTTGTTCACCAAGAGAC |
| | | CGTAACTGTGATTGACTCCAGATGTGGCGAGAATTTTTCTATTTATAAGGCATCAGATAATTCTTCTTTGAGTCAACAGTTCGATGCTTGC |
| | | GCAAGTTGGTGGACACAGGGGCCTGACCCTACTTTTCAGGCTGAATTGGCTCGCGAGATGGGATACACAGCGGCCAGGTTCGGCCAT |
| | | GTAATGTTCCCCGAAAATGTTTATGAACCTGCTCTTAAATGTGCTGAGCTTTTGTTAGACGGTGTAGGAAAGGGATGGGCTTCACGCGT |
| | | ATACTTCTCTGATAATGGCTCAACCGCAATAGAGATCGCTTTGAAGATGGCTTTTAGGAAATTTTGCGTTGACCACAACTTTTGCGAAGC |
| | | CACAGAGGAAGAGAAACACATTGTGGTTAAGGTAATTGCCCTGAGAGGCTCATACCACGGCGATACTTTGGGTGCTATGGAGGCTCAG |
| | | GCGCCATCACCATATACTGGTTTTCTTCAGCAACCCTGGTACACGGGCCGGGGCCTATTTTTGGATCCTCCAACCGTTTTTTTGTCTAA |
| | | CGGTTCTTGGAACATATCTTTGCCGGAAAGTTTCTCAGAAATTGCTCCTGAGTACGGCACCTTCACAAGTCGTGATGAAATCTTCGATAA |
| | | GTCAAGAGACGCTTCAACACTTGCAAGAATCTATTCAGCTTATCTCTCAAAGCATTTGCAGGAGCATTCAGGAGTTAGGCAGAGTGCCC |
| | | ATGTTGGTGCATTAATCATTGAGCCGGTGATTCATGGTGCAGGAGGAATGCATATGGTGGATCCTCTATTTCAGAGAGTCCTCGTTAAT |
| | | GAATGTAGAAATCGTAAGATACCTGTCATATTTGACGAAGTCTTCACTGGTTTTTGGAGATTGGGAGTGGAAACGACAACAGAACTTCTA |
| | | GGATGCAAACCTGATATTGCGTGCTTTGCTAAGCTATTGACGGGCGGTATGGTACCCTTGGCTGTGACATTGGCTACAGATGCAGTTTT |
| | | CGATAGTTTTAGTGGTGATAGCAAATTGAAAGCTCTATTGCACTCTCATAGTTACTCTGCTCACGCCATGGGATGCGCTACCGCGGCGA |
| | | AGGCAATCCAATGGTTTAAAGATCCTGAAACCAATCACAATATCACATCTCAAGGCAAGACCCTCAGGGAACTGTGGGACGAAGAACTA |
| | | GTTCAGCAAATCTCTTCACACTCAGCAGTGCAACGCGTTGTTGTCATAGGAACCTTGTTTGCTCTCGAGTTGAAGGCAGACGCAAGCAA |
| | | CTCAGGGTACGCTTCACTCTATGCTAAATCTTTATTGATTATGTTGAGGGAAGATGGCATCTTTACACGACCTCTGGGTAACGTGATCTA |
| | | CCTCATGTGCGGTCCTTGTACGAGTCCCGAAATATGTAGACGACTGCTGACTAAACTGTATAAGCGTTTGGGTGAATTTAATCGTACTTA |
| | | A |
| 346 | Zea mays BioDA nucleic | ATGGTTAGACGCCTGTTTCTTCACCATGCTCGGAGGAGATTGCATTCAGGTACCGCATCAAGTATTCCTCTTTCTACTCCTATATTTGCG |
| | acid C372D (SEQ ID | ATATTCGGGGCTAATACGGGCGTAGGAAAGACGTTAGTCAGTGCTGGGTTGGCTACTGCACTATTATCTTCTCCATCCCCTGCAGTTTC |
| | NO: 346) | AAGTGTTGCTTACTTGAAGCCACTACAAACTGGCTATCCTGTGGACTCAGACGCGAGCTTTGTATTCTCTAGGACACCAGCTTTACTTAG |
| | | AGCATTTCCACCTTGCCGTGCCACCAGGCTCATAGCTAGTTGTCGTACATTCTTCCAATCACCAGCTGTCGAAGCTAAAGCAGAGTCAT |
| | | TGCATGAATCACAGGAAAAAGTGGTGACGTACGGCGCAGGGGCTGCAGAGCAGACTAAGGTCCTGGCATGCTGTACAGCTTACGCCT |
| | | GGCGAGAGCCGGTCTCCCCACACCTAGCAGCCGAGAGAGAGGGCATGGCTGCTGGCGATGATGAAGTAAAGGGATGTGTGGAACAA |
| | | TGGCTATTGGAAGAGGGTATCGGTGAAGGTGGAGAGGTGTGGAAAATACTGGAGACTGCTGGTGGTGTAGCTAGCCCTAGCGCTTCT |
| | | GGTACGTTACAATGTGATCTCTATAGGCCATTTCGTCTTCCTGCAATTCTTGTAGGTGATGGACGATTGGGGGGTATTAGTTCTACTTTG |
| | | TCAGCCTATGAAACACTTCTCTTGAGGGGCTATGATGTCTCAGCCGTCATCCTGGAAGACCGTGAGTTATCTAATGATAAGTTTCTTCTG |
| | | TCATACCTTAGGAATCGTGTTCATGTTTTAATCTTGCCCCAAATACCAGAGGATCCACTTGATGACTTGACCGATTGGTTCAGTGAATCT |
| | | TCAAGCATATTTATATTTTTAAAAGATGAATTGCAAAGTTTCCATTCAAGACGTATTGAAAGACTTAATAGCATGCAAAGGAAGAGTAAAG |
| | | CGTTGCTGTGGTGGCCATTTACCCAGCACAACTTGGTTCCCCAGGATTCTGTTACAGTTATTGATAGCAGATATGGTGAAAATTTCTCCG |
| | | TTTACAAGATTAAGGATAAGATGCTTATACCTCAATTCGATGCAGATGCTAGTTGGTGGACTCAAGGTCCTGACTCCAATCTTCAAATTG |
| | | AGCTTGCTAGAGATATGGGATATGCTGCTGCTAGATATGGTCATGTTATGTTTCCTGAGAATGTTCATGAACCTGCTCTCCATAGTGCTG |
| | | AGGTACTCCTCGGTGGTGTGGGAAAGGGTTGGGCGAGCCGTGTTTATTATAGCGATAATGGGAGCACCGCTATTGAGATCGCTTTAAA |
| | | AATGGCATTTAGGAAATTTTCTTTAGACCACGGTATCATGGCTTCATCTGAAAACTCTACTCGCAACGAAAGAAATATTCAACTCAAAGTT |
| | | TTAGCACTAAATGGATCTTATCACGGTGATACGCTTGGAGCAATGGAAGCGCAAGCTCCGTCCGCTTACACCAGTTTCCTCCAACAGCC |
| | | TTGGTACAGCGGTAGGGGACTTTTTCTCGACCCTCCAACCGTATATATTAAGTCAGAGGTGTATAACATTTCCTTGCCTCAATCTATGCA |
| | | GCATGATCCACAAACATATGGGTTTTTTTCACAGGCCGAAGTTTTTTGCAAGACACGAGATAAGACATTTGCGGCTGATCTGTATTCATC |
| | | TTATATAAAGCAAAAATTGTCAGAGTTTTCTCTTTCCTCATCATCAGAGCATTTAGCCGCCTTGATTATTGAACCTGTTATACAAGGGGCT |
| | | GGCGGTATGCTGATGATTGATCCGTTATTCCAGAGAGTGCTTGTTTCAGAATGTAGATCAAGGAAGATACCTGTTATTTTTGATGAGGTC |
| | | TTTACGGGATTCTGGCGTCTAGGTGTTGAGAGCGCGTCCGAGCTTCTTGGATGCCTACCTGATATCGCTTGTTACGCCAAGCTTATGAC |
| | | TGGGGGGATTATCCCATTAGCCGCTACTCTTGCTACTGAGGAGGTTTTTGAAAGTTTCGAGAGCGACTCAAAATTGACCGCCCTCCTGC |
| | | ATGGGCACTCTTATACTGCACATGCAATGGGATGTACTGCAGCTTTAAAAGCTATGCAGTGGTATAGGGGACCAAGCACGAATTCAAAC |
| | | TTGGATACAGATCATATGAAATTAAAGGAATTATGGGACGGCACTCTGGTCAAACAACTCTCCAGCCTGCCTAACGTGAAAAGAGTTGT |
| | | CTCACTTGGGACTCTTTGTGCAATAGAGCTAAAAGCAGAAGGTGCTGATGCGGGATATGCTTCACTTTACGCTTCAAGTTTAGTACAAC |
| | | AATTAAGGAAGGAAGATGATATCTACATTCGCCCACTTGGGAATGTCATTTACCTTATGTGCGGACCTTGTACCCCACGTGATAGTTGCA |
| | | CACGTCAGCTTCTCAAGGTCCATAGAAGATTGTGCGCTCTCAACTAA |
| 347 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A507K | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQKPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 348 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A507S | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQSPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 349 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609C | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGCGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 350 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609F | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGFGGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 351 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609H | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGHGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 352 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609I | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGIGGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 353 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609M | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGMGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 354 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609N (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGNGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 355 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609T (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGTGGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 356 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609V (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGVGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 357 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609W (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGWGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 358 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | C388D (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDADASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 359 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | C388T (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDATASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 360 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348C (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPCTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 361 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348D (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPDTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 362 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348N (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPNTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 363 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348S (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPSTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 364 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348T (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPTTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 365 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348V (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPVTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 366 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G513DEL (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTFLQQPWYTGRGLFLDPPTVFLSNGSWNISLPESFSEIAPEYGTFTSRDEIFDKSRDASTLARIYSAYLS |
| | | KHLQEHSGVRQSAHVGALIIEPVIHGAGGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 367 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G513L (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTLFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 368 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G513P (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTPFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 369 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G608E (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHEAGGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 370 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G700A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHAHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 371 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G700S (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHSHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 372 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | L786S (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLSIMLREDGIFTRPLGNVIYLMCGPCTSPEICRRLLTKLYKRLGEFNRT |
| 373 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P421L (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFLENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 374 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P508DEL (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQASPYTGFLQQPWYTGRGLFLDPPTVFLSNGSWNISLPESFSEIAPEYGTFTSRDEIFDKSRDASTLARIYSAYLS |
| | | KHLQEHSGVRQSAHVGALIIEPVIHGAGGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 375 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P508L (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQALSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 376 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P508T (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQATSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 377 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P510L (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSLYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 378 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q506A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAAAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 379 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q516C (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLCQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 380 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q517D (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQDPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 381 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q517F (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQFPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 382 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q517M (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQMPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 383 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q517T (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQTPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 384 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q517Y (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQYPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 385 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | R756S (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQSVVVIGTLFALE |
| | | LKADASNSGYASLYAKSLLIMLREDGIFTRPLGNVIYLMCGPCTSPEICRRLLTKLYKRLGEFNRT |
| 386 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509C (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPCPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 387 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509D (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPDPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 388 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509E (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPEPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 389 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509G (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPGPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 390 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509V (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPVPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 391 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509W (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPWPYTGFLQQPWYTGRGLFLDPPTVFLSNGSWNISLPESFSEIAPEYGTFTSRDEIFDKSRDASTLARIYSAY |
| | | LSKHLQEHSGVRQSAHVGALIIEPVIHGAGGMHMVDPLFQRVLVNECRNRKIPVIFDEVFTGFWRLGVETTTELLGCKPDIACFAKLLTGGMV |
| | | PLAVTLATDAVFDSFSGDSKLKALLHGHSYSAHAMGCATAAKAIQWFKDPETNHNITSQGKTLRELWDEELVQQISSHSAVQRVVVIGTLFAL |
| | | ELKADASNSGYASLYAKSLLIMLREDGIFTRPLGNVIYLMCGPCTSPEICRRLLTKLYKRLGEFNRT |
| 392 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S704H (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYHAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 393 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | T512C (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYCGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 394 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | T512D (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYDGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 395 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | T512Q (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYQGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 396 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | T512R (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYRGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 397 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | T512V (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYVGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 398 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | W391L (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASLWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 399 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | W391M (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASMWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 400 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | W392A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWATQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 401 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | W392G (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWGTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 402 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | W392S (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWSTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 403 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511D (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPDTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 404 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511E (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPETGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 405 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511F (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPFTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 406 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511M (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPMTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 407 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511P (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPPTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 408 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511R (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPRTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 409 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511V (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPVTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 410 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511W (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPWTGFLQQPWYTGRGLFLDPPTVFLSNGSWNISLPESFSEIAPEYGTFTSRDEIFDKSRDASTLARIYSAY |
| | | LSKHLQEHSGVRQSAHVGALIIEPVIHGAGGMHMVDPLFQRVLVNECRNRKIPVIFDEVFTGFWRLGVETTTELLGCKPDIACFAKLLTGGMV |
| | | PLAVTLATDAVFDSFSGDSKLKALLHGHSYSAHAMGCATAAKAIQWFKDPETNHNITSQGKTLRELWDEELVQQISSHSAVQRVVVIGTLFAL |
| | | ELKADASNSGYASLYAKSLLIMLREDGIFTRPLGNVIYLMCGPCTSPEICRRLLTKLYKRLGEFNRT |
| 411 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A389F (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACFSWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 412 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609K (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGKGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 413 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609R (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGRGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 414 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | A609Y (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGYGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 415 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | C388M (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDAMASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 416 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPATQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 417 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348E (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPETQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 418 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348I (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPITQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 419 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348K (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPKTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 420 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348M (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPMTQHKLVHQETVTVIDSRCGENFSIYKASDNSSLSQQFDACASWWTQ |
| | | GPDPTFQAELAREMGYTAARFGHVMFPENVYEPALKCAELLLDGVGKGWASRVYFSDNGSTAIEIALKMAFRKFCVDHNFCEATEEEKHIVV |
| | | KVIALRGSYHGDTLGAMEAQAPSPYTGFLQQPWYTGRGLFLDPPTVFLSNGSWNISLPESFSEIAPEYGTFTSRDEIFDKSRDASTLARIYSAY |
| | | LSKHLQEHSGVRQSAHVGALIIEPVIHGAGGMHMVDPLFQRVLVNECRNRKIPVIFDEVFTGFWRLGVETTTELLGCKPDIACFAKLLTGGMV |
| | | PLAVTLATDAVFDSFSGDSKLKALLHGHSYSAHAMGCATAAKAIQWFKDPETNHNITSQGKTLRELWDEELVQQISSHSAVQRVVVIGTLFAL |
| | | ELKADASNSGYASLYAKSLLIMLREDGIFTRPLGNVIYLMCGPCTSPEICRRLLTKLYKRLGEFNRT |
| 421 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F348Q (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPQTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 422 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F370L (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPLTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 423 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | F420I (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPITQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 424 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G513A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTAFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 425 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G608A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHAAGGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 426 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G608I (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHIAGGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 427 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G610H (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAHGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 428 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | G700C (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHCHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 429 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | H496S (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYSGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 430 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | M419I (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVIFPENVYEPALKCAELLLDGVGKGWASRVYFSDNGSTAIEIALKMAFRKFCVDHNFCEATEEEKHIVVKVI |
| | | ALRGSYHGDTLGAMEAQAPSPYTGFLQQPWYTGRGLFLDPPTVFLSNGSWNISLPESFSEIAPEYGTFTSRDEIFDKSRDASTLARIYSAYLS |
| | | KHLQEHSGVRQSAHVGALIIEPVIHGAGGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 431 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | M612L (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGLHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 432 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P347A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWAFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 433 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P347E (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWEFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 434 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P421A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFAENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 435 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P421E (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFEENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRTNRT |
| 436 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P421W (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFWENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 437 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P510A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSAYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 438 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P510C (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSCYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 439 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P510E (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSEYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 440 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P510Q (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSQYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 441 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P510V (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSVYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 442 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P529A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDAPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 443 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q350H (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTHHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 444 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q350S (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTSHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 445 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q517H (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQHPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 446 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q517I (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQIPWYTGRGLFLDPPTVFLSNGSWNISLPESFSEIAPEYGTFTSRDEIFDKSRDASTLARIYSAYLS |
| | | KHLQEHSGVRQSAHVGALIIEPVIHGAGGMHMVDPLFQRVLVNECRNRKIPVIFDEVFTGFWRLGVETTTELLGCKPDIACFAKLLTGGMVPL |
| | | AVTLATDAVFDSFSGDSKLKALLHGHSYSAHAMGCATAAKAIQWFKDPETNHNITSQGKTLRELWDEELVQQISSHSAVQRVVVIGTLFALEL |
| | | KADASNSGYASLYAKSLLIMLREDGIFTRPLGNVIYLMCGPCTSPEICRRLLTKLYKRLGEFNRT |
| 447 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Q517W (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQWPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 448 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | R756K (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQKVVVIGTLFALE |
| | | LKADASNSGYASLYAKSLLIMLREDGIFTRPLGNVIYLMCGPCTSPEICRRLLTKLYKRLGEFNRT |
| 449 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | R790C (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLCEDGIFTRPLGNVIYLMCGPCTSPEICRRLLTKLYKRLGEFNRT |
| 450 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | R797Q (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTQPLGNVIYLMCGPCTSPEICRRLLTKLYKRLGEFNRT |
| 451 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S390C (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACACWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 452 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S494A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGAYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 453 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPAPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 454 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509F (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPFPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 455 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509H (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPHPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 456 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509I (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPIPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 457 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509K (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPKPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 458 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509L (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPLPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 459 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509M (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPMPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 460 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509N (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPNPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 461 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509Q (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPQPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 462 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509R (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPRPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 463 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509T (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPTPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 464 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S509Y (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPYPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 465 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | S704P (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYPAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 466 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | T393V (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWVQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 467 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | T512G (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYGGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 468 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | T512I (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYIGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 469 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | T512N (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYNGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 470 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | T512W (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYWGFLQQPWYTGRGLFLDPPTVFLSNGSWNISLPESFSEIAPEYGTFTSRDEIFDKSRDASTLARIYSAY |
| | | LSKHLQEHSGVRQSAHVGALIIEPVIHGAGGMHMVDPLFQRVLVNECRNRKIPVIFDEVFTGFWRLGVETTTELLGCKPDIACFAKLLTGGMV |
| | | PLAVTLATDAVFDSFSGDSKLKALLHGHSYSAHAMGCATAAKAIQWFKDPETNHNITSQGKTLRELWDEELVQQISSHSAVQRVVVIGTLFAL |
| | | ELKADASNSGYASLYAKSLLIMLREDGIFTRPLGNVIYLMCGPCTSPEICRRLLTKLYKRLGEFNRT |
| 471 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | V354A (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLAHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 472 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | V354E (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLEHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 473 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | V354L (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLLHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 474 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | V354N (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLNHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 475 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | V354T (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLTHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 476 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | W391F (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASFWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 477 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | W392C (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWCTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 478 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | W392D (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWDTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 479 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | W392M (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWMTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 480 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511C (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPCTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 481 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511H (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPHTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 482 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511I (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPITGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 483 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511K (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPKTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 484 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y511Q (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPQTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 485 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y520N (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWNTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 486 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | Y520W (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFPENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWWTGRGLFLDPPTVFLSNGSWNISLPESFSEIAPEYGTFTSRDEIFDKSRDASTLARIYSAY |
| | | LSKHLQEHSGVRQSAHVGALIIEPVIHGAGGMHMVDPLFQRVLVNECRNRKIPVIFDEVFTGFWRLGVETTTELLGCKPDIACFAKLLTGGMV |
| | | PLAVTLATDAVFDSFSGDSKLKALLHGHSYSAHAMGCATAAKAIQWFKDPETNHNITSQGKTLRELWDEELVQQISSHSAVQRVVVIGTLFAL |
| | | ELKADASNSGYASLYAKSLLIMLREDGIFTRPLGNVIYLMCGPCTSPEICRRLLTKLYKRLGEFNRT |
| 487 | Arabidopsis thaliana | MKSTSVSPFHLPLNHPTYLIWSANTSLGKTLVSTGIAASFLLQQPSSSATKLLYLKPIQTGFPSDSDSRFVFSKLDSLSLRRQIPISISNSVLHSS |
| | BioDAΔMTP protein | LPAAKSLGLNVEVSESGMCSLNFRDEKTVTGAPELLCKTLYAWEAAISPHLAAERENATVEDSVVLQMIEKCLKEEMECGVKSEKSDLLCLVE |
| | P421G (SEQ ID NO: | TAGGVASPGPSGTLQCDLYRPFRLPGILVGDGRLGGISGTIAAYESLKLRGYDIAAVVFEDHGLVNEVPLTSYLRNKVPVLVLPPVPKDPSDDL |
| | | IEWFVESDGVFKALKETMVLANLERLERLNGMAKLAGEVFWWPFTQHKLVHQETVTVIDSRCGENFSIYKASDNSSLSQQFDACASWWTQG |
| | | PDPTFQAELAREMGYTAARFGHVMFGENVYEPALKCAELLLDGVGKGWASRVYFSDNGSTAIEIALKMAFRKFCVDHNFCEATEEEKHIVVK |
| | | VIALRGSYHGDTLGAMEAQAPSPYTGFLQQPWYTGRGLFLDPPTVFLSNGSWNISLPESFSEIAPEYGTFTSRDEIFDKSRDASTLARIYSAYL |
| | | SKHLQEHSGVRQSAHVGALIIEPVIHGAGGMHMVDPLFQRVLVNECRNRKIPVIFDEVFTGFWRLGVETTTELLGCKPDIACFAKLLTGGMVP |
| | | LAVTLATDAVFDSFSGDSKLKALLHGHSYSAHAMGCATAAKAIQWFKDPETNHNITSQGKTLRELWDEELVQQISSHSAVQRVVVIGTLFALE |
| | | LKADASNSGYASLYAKSLLIMLREDGIFTRPLGNVIYLMCGPCTSPEICRRLLTKLYKRLGEFNRT |
| 488 | Arabidopsis thaliana | ATGATTCCCGTGACCGCCACTCTTATTCGTCACAGGTTGAGGCACTTGCGTCATCGAATTAGGTTTAAATCAACCTCTGTATCACCATTC |
| | BioDA nucleic acid | CATTTGCCGCTCAATCATCCTACTTATTTGATTTGGTCTGCCAACACTAGCCTGGGCAAAACTTTGGTCAGTACGGGGATAGCAGCCAG |
| | P421L (SEQ ID NO: | TTTTCTCTTACAACAACCTAGTTCATCAGCAACAAAACTATTGTATCTCAAACCCATCCAGACAGGTTTTCCTAGTGATTCTGACTCAAGA |
| | | TTTGTTTTTTCAAAGCTCGACTCATTGTCATTGCGAAGACAAATTCCTATAAGTATCTCAAATTCCGTACTTCATTCATCACTCCCTGCTG |
| | | CTAAGTCTCTTGGCCTAAATGTTGAAGTAAGCGAGTCTGGAATGTGTAGTCTCAATTTTCGGGATGAGAAAACTGTTACCGGCGCTCCA |
| | | GAGTTGTTGTGCAAGACACTTTATGCTTGGGAGGCTGCAATTTCACCCCATCTTGCTGCAGAGAGAGAAAACGCAACTGTTGAAGACTC |
| | | TGTGGTTCTTCAAATGATTGAGAAGTGCCTGAAGGAGGAGATGGAGTGCGGAGTTAAATCTGAGAAGAGTGATCTCTTGTGTCTAGTCG |
| | | AAACTGCTGGCGGAGTTGCTTCTCCTGGTCCATCTGGAACCTTGCAATGTGACCTGTATAGGCCGTTTAGGCTCCCCGGAATTCTAGTT |
| | | GGAGACGGCAGGTTGGGGGGTATCTCTGGCACAATAGCAGCCTATGAGAGTCTCAAGCTTCGTGGATACGATATCGCTGCTGTTGTCT |
| | | TCGAAGATCACGGACTAGTTAACGAAGTGCCACTTACATCATATTTGAGGAATAAGGTACCTGTCCTCGTATTACCTCCTGTCCCTAAAG |
| | | ACCCTTCAGACGACTTGATCGAATGGTTCGTGGAGTCCGATGGCGTATTCAAAGCACTTAAGGAGACGATGGTATTAGCGAACCTTGAA |
| | | CGTCTTGAAAGGTTGAATGGTATGGCCAAACTGGCTGGTGAAGTGTTTTGGTGGCCATTCACACAGCATAAGCTTGTTCACCAAGAGAC |
| | | CGTAACTGTGATTGACTCCAGATGTGGCGAGAATTTTTCTATTTATAAGGCATCAGATAATTCTTCTTTGAGTCAACAGTTCGATGCTTGC |
| | | GCAAGTTGGTGGACACAGGGGCCTGACCCTACTTTTCAGGCTGAATTGGCTCGCGAGATGGGATACACAGCGGCCAGGTTCGGCCAT |
| | | GTAATGTTCCTTGAAAATGTTTATGAACCTGCTCTTAAATGTGCTGAGCTTTTGTTAGACGGTGTAGGAAAGGGATGGGCTTCACGCGTA |
| | | TACTTCTCTGATAATGGCTCAACCGCAATAGAGATCGCTTTGAAGATGGCTTTTAGGAAATTTTGCGTTGACCACAACTTTTGCGAAGCC |
| | | ACAGAGGAAGAGAAACACATTGTGGTTAAGGTAATTGCCCTGAGAGGCTCATACCACGGCGATACTTTGGGTGCTATGGAGGCTCAGG |
| | | CGCCATCACCATATACTGGTTTTCTTCAGCAACCCTGGTACACGGGCCGGGGCCTATTTTTGGATCCTCCAACCGTTTTTTTGTCTAAC |
| | | GGTTCTTGGAACATATCTTTGCCGGAAAGTTTCTCAGAAATTGCTCCTGAGTACGGCACCTTCACAAGTCGTGATGAAATCTTCGATAAG |
| | | TCAAGAGACGCTTCAACACTTGCAAGAATCTATTCAGCTTATCTCTCAAAGCATTTGCAGGAGCATTCAGGAGTTAGGCAGAGTGCCCA |
| | | TGTTGGTGCATTAATCATTGAGCCGGTGATTCATGGTGCAGGAGGAATGCATATGGTGGATCCTCTATTTCAGAGAGTCCTCGTTAATG |
| | | AATGTAGAAATCGTAAGATACCTGTCATATTTGACGAAGTCTTCACTGGTTTTTGGAGATTGGGAGTGGAAACGACAACAGAACTTCTAG |
| | | GATGCAAACCTGATATTGCGTGCTTTGCTAAGCTATTGACGGGGGGTATGGTACCCTTGGCTGTGACATTGGCTACAGATGCAGTTTTC |
| | | GATAGTTTTAGTGGTGATAGCAAATTGAAAGCTCTATTGCACGGACATAGTTACTCTGCTCACGCCATGGGATGCGCTACCGCGGCGAA |
| | | GGCAATCCAATGGTTTAAAGATCCTGAAACCAATCACAATATCACATCTCAAGGCAAGACCCTCAGGGAACTGTGGGACGAAGAACTAG |
| | | TTCAGCAAATCTCTTCACACTCAGCAGTGCAACGCGTTGTTGTCATAGGAACCTTGTTTGCTCTCGAGTTGAAGGCAGACGCAAGCAAC |
| | | TCAGGGTACGCTTCACTCTATGCTAAATCTTTATTGATTATGTTGAGGGAAGATGGCATCTTTACACGACCTCTGGGTAACGTGATCTAC |
| | | CTCATGTGCGGTCCTTGTACGAGTCCCGAAATATGTAGACGACTGCTGACTAAACTGTATAAGCGTTTGGGTGAATTTAATCGTACTTAA |

1. A method of controlling undesired vegetation in the vicinity of a plant or part thereof, or at the locus for growth of a plant or part thereof, wherein the plant or part thereof is modified to comprise a BIO3-BIO1 enzyme and/or BioA enzyme that provides the plant or part thereof with increased resistance to a compound which inhibits the biotin synthesis pathway relative to an unmodified reference plant, the method comprising: applying an effective amount of at least one compound which inhibits the biotin synthesis pathway to the undesired vegetation and the plant, or the locus, and planting a seed at the locus, wherein the seed is capable of producing the plant.

2. A method according to claim 1, wherein the plant or a part thereof is modified to comprise a polynucleotide encoding a BIO3-BIO1 and/or BioA enzyme, the expression of which provides the plant or part thereof with increased resistance to a compound which inhibits the biotin synthesis pathway relative to an unmodified plant.

3. A method according to claim 1, wherein the plant or part thereof is modified to increase expression of the BIO3-BIO1 enzyme and/or a BioA enzyme, preferably wherein the plant or part thereof is modified to overexpress the BIO3-BIO1 enzyme and/or a BioA enzyme.

4. A method according to claim 2, wherein the expression of the BIO3-BIO1 enzyme and/or a BioA enzyme is increased or overexpressed to a level which provides the plant or part thereof with increased resistance to a compound which inhibits the biotin synthesis pathway relative to an unmodified plant, preferably wherein the expression of the BIO3-BIO1 enzyme and/or a BioA enzyme is increased or overexpressed to a level of at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% greater than the expression of the BIO3-BIO1 enzyme and/or a BioA enzyme in an unmodified reference plant.

5. A method according to claim 1, wherein the BIO3-BIO1 enzyme and/or the BioA enzyme is a wild type enzyme, and/or wherein the BIO3-BIO1 enzyme and/or the BioA enzyme is heterologous to the plant or part thereof.

6. A method according to claim 1, wherein the BIO3-BIO1 enzyme comprises or consists of a sequence having at least 30% identity to any of SEQ ID NOs 1 to 14, 271-276, 319, or a functional fragment thereof.

7. A method according to claim 1, wherein the BioA enzyme comprises or consists of a sequence having at least 30% identity to any of SEQ ID NOs 159-199, or a functional fragment thereof.

8. A method according to claim 1, wherein the BioA enzyme comprises the following motif: (H/Q); (C/I/M/S/T/V); (I/L/M/T/V); (F/L/Y); (A/G/L/P); (D/E/G/M/N); (A/C/F/I/L/M/N/Y); (A/G/I/L/S/T/V); (H/N/Y); (E/K/N/P/Q/S/V); (A/G/K/Q/P/T) (Motif 17) (SEQ ID NO: 270).

9. A method according to claim 1, wherein the BioA enzyme comprises or consists of a sequence having at least 70% identity to any of SEQ ID NOs 160-199, or a functional fragment thereof.

10. A method according to claim 1, wherein the BioA enzyme comprises or consists of a sequence having at least 70% identity to SEQ ID NO:167 or 170.

11. A method according to claim 1, wherein the plant or part thereof is modified to comprise a BIO3-BIO1 enzyme and/or BioA enzyme having one or more mutations, preferably wherein the or each mutation is an amino acid substitution.

12. A method according to claim 9, wherein the or each mutation provides the plant or part thereof with increased resistance to a compound which inhibits the biotin synthesis pathway relative to an unmodified plant.

13. A method according to claim 1, wherein the BIO3-BIO1 enzyme comprises an amino acid sequence having at least 30% identity to SEQ ID NO:1, and comprises an amino acid substitution at one or more of the following positions: F348, P347,Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, and R797 of SEQ ID NO:1, or at corresponding positions thereto.

14. A method according to claim 1, wherein the BIO3-BIO1 enzyme and/or the BioA enzyme is modified to comprise a heterologous targeting peptide, preferably a heterologous mitochondrial targeting peptide.

15. A method according to claim 1, wherein the compound is a herbicide, preferably wherein the compound is a herbicide selected from: a herbicidal cinnoline compound such as ethyl 5-acetyl-4-oxo-1-(2,2,3,3-tetrafluoro-1,4-benzodioxin-6-yl)cinnoline-3-carboxylate (Compound A); a herbicidal compound such as [5-carboxy-1-(1-hydroxyethyl)pentyl]ammonium;chloride (compound B); a herbicidal pyridone compound such as 2-(3,4-dichlorophenyl)-5-(2,4-difluorophenyl)-1-ethyl-6-methyl-4-oxo-pyridine-3-carboxylic acid (Compound C) or 5-[2-chloro-5-(trifluoromethoxy)phenyl]-2-(3,4-dichlorophenyl)-1-ethyl-6-methyl-4-oxo-pyridine-3-carboxylic acid (Compound D); a herbicidal compound such as [(1R)-2-(6-carboxypyridin-1-ium-2-yl)oxy-1-methyl-ethyl]ammonium;dichloride (Compound E); a herbicidal pyrrolidine-2-one such as 2-[1-[(2,3-difluorophenyl)methyl]-5-oxo-pyrrolidin-2-yl]-N-(2-methyl-1,2,4-triazol-3-yl)acetamide (Compound F), 2-[1-[(2,3-difluorophenyl)methyl]-5-oxo-pyrrolidin-2-yl]acetic acid (Compound G), 2-[5-oxo-1-[(2,3,5-trifluorophenyl)methyl]pyrrolidin-2-yl]acetic acid (Compound H), 2-(4-fluorophenoxy)ethyl 2-[1-[(2,3-difluorophenyl)methyl]-5-oxo-pyrrolidin-2-yl]acetate (Compound I), 2-methoxyethyl 2-[1-[(2,3-difluorophenyl)methyl]-5-oxo-pyrrolidin-2-yl]acetate (Compound J), 2-[5-oxo-1-[(2,3,4-trifluorophenyl)methyl]pyrrolidin-2-yl]acetic acid (Compound K); a N-benzyl azole herbicidal compound such as 2-(3,4-dichlorophenyl)-1-ethyl-4-oxo-6-[[3-(trifluoromethyl)pyrazol-1-yl]methyl]pyridine-3-carboxylic acid (Compound L); and a herbicidal quinolone compound such as 2-(3,4-dichlorophenyl)-1-ethyl-6-fluoro-4-oxo-quinoline-3-carboxylic acid (Compound M).

16. A plant, or part thereof, modified to comprise a BIO3-BIO1 enzyme that provides the plant or part thereof with increased resistance to a compound which inhibits the biotin synthesis pathway relative to an unmodified reference plant, wherein the BIO3-BIO1 enzyme comprises an amino acid sequence having at least 30% identity to SEQ ID NO:1, and comprises an amino acid substitution at one or more of the following positions: F348, P347, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, and R797 of SEQ ID NO:1, or at corresponding positions thereto.

17. A plant, or part thereof, modified to comprise a BioA enzyme that provides the plant or part thereof with increased resistance to a compound which inhibits the biotin synthesis pathway relative to an unmodified reference plant, wherein the BioA enzyme comprises the following motif: (H/Q); (C/I/M/S/T/V); (I/L/M/T/V); (F/L/Y); (A/G/L/P); (D/E/G/M/N); (A/C/F/I/L/M/N/Y); (A/G/I/L/S/T/V); (H/N/Y); (E/K/N/P/Q/S/V); (A/G/K/Q/P/T) (Motif 17) (SEQ ID NO: 270).

18. A plant or part thereof according to claim 17, wherein the BioA enzyme comprises or consists of a sequence having at least 70% identity to any of SEQ ID NOs 160-199, or a functional fragment thereof.

19. A plant, or part thereof according to claim 17, wherein the BioA enzyme comprises or consists of a sequence having at least 70% identity to SEQ ID NO: 167 or 170.

20. A plant, or part thereof, according to claim 16, wherein the plant or a part thereof is modified to comprise a polynucleotide encoding a BIO3-BIO1 and/or BioA enzyme, the expression of which provides the plant or part thereof with increased resistance to a compound which inhibits the biotin synthesis pathway relative to an unmodified plant.

21. A plant, or part thereof, according to claim 16 wherein the plant or part thereof is modified to increase expression of the BIO3-BIO1 enzyme and/or a BioA enzyme, preferably wherein the plant or part thereof is modified to overexpress the BIO3-BIO1 enzyme and/or a BioA enzyme.

22. A plant, or part thereof, according to claim 16, wherein the expression of the BIO3-BIO1 enzyme and/or a BioA enzyme is increased or overexpressed to a level which provides the plant or part thereof with increased resistance to a compound which inhibits the biotin synthesis pathway relative to an unmodified plant, preferably wherein the expression of the BIO3-BIO1 enzyme and/or a BioA enzyme is increased or overexpressed to a level of at least 5%, at least 10%, at least 20%, at least 30%, at least 40%, at least 50%, at least 60%, at least 70%, at least 80%, at least 90%, at least 100%, at least 110%, at least 120%, at least 130%, at least 140%, at least 150% greater than the expression of the BIO3-BIO1 enzyme and/or a BioA enzyme in an unmodified reference plant.

23. A plant, or part thereof, according to claim 16, wherein the BIO3-BIO1 enzyme and/or the BioA enzyme is heterologous to the plant or part thereof.

24. A plant, or part thereof, according to claim 16, wherein the BIO3-BIO1 enzyme and/or the BioA enzyme is modified to comprise a heterologous targeting peptide, preferably a heterologous mitochondrial targeting peptide.

25. A plant, or part thereof according to claim 16, wherein the compound is a herbicide, preferably wherein the compound is a herbicide selected from: a herbicidal cinnoline compound such as ethyl 5-acetyl-4-oxo-1-(2,2,3,3-tetrafluoro-1,4-benzodioxin-6-yl)cinnoline-3-carboxylate (Compound A); a herbicidal compound such as [5-carboxy-1-(1-hydroxyethyl)pentyl]ammonium;chloride (compound B); a herbicidal pyridone compound such as 2-(3,4-dichlorophenyl)-5-(2,4-difluorophenyl)-1-ethyl-6-methyl-4-oxo-pyridine-3-carboxylic acid (Compound C) or 5-[2-chloro-5-(trifluoromethoxy)phenyl]-2-(3,4-dichlorophenyl)-1-ethyl-6-methyl-4-oxo-pyridine-3-carboxylic acid (Compound D); a herbicidal compound such as [(1R)-2-(6-carboxypyridin-1-ium-2-yl)oxy-1-methyl-ethyl]ammonium;dichloride (Compound E); a herbicidal pyrrolidine-2-one such as 2-[1-[(2,3-difluorophenyl)methyl]-5-oxo-pyrrolidin-2-yl]-N-(2-methyl-1,2,4-triazol-3-yl)acetamide (Compound F), 2-[1-[(2,3-difluorophenyl)methyl]-5-oxo-pyrrolidin-2-yl]acetic acid (Compound G), 2-[5-oxo-1-[(2,3,5-trifluorophenyl)methyl]pyrrolidin-2-yl]acetic acid (Compound H), 2-(4-fluorophenoxy)ethyl 2-[1-[(2,3-difluorophenyl)methyl]-5-oxo-pyrrolidin-2-yl]acetate (Compound I), 2-methoxyethyl 2-[1-[(2,3-difluorophenyl)methyl]-5-oxo-pyrrolidin-2-yl]acetate (Compound J), 2-[5-oxo-1-[(2,3,4-trifluorophenyl)methyl]pyrrolidin-2-yl]acetic acid (Compound K); a N-benzyl azole herbicidal compound such as 2-(3,4-dichlorophenyl)-1-ethyl-4-oxo-6-[[3-(trifluoromethyl)pyrazol-1-yl]methyl]pyridine-3-carboxylic acid (Compound L); and a herbicidal quinolone compound such as 2-(3,4-dichlorophenyl)-1-ethyl-6-fluoro-4-oxo-quinoline-3-carboxylic acid (Compound M).

26. A modified BIO3-BIO1 enzyme having one or more mutations which provide increased resistance to a compound which inhibits the biotin synthesis pathway relative to an unmodified reference BIO3-BIO1 enzyme, wherein the modified BIO3-BIO1 enzyme comprises an amino acid sequence having at least 30% identity to SEQ ID NO:1, and comprises an amino acid substitution at one or more of the following positions: F348, P347, Q350, V354, F370, C388, A389, S390, W391, W392, T393, M419, F420, P421, Q506, A507, P508, S509, P510, Y511, T512, G513, Q516, Q517, Y520, P529, G608, A609, G610, M612, G700, S704, R756, L786, R790, and R797 of SEQ ID NO:1, or at corresponding positions thereto.

27. A modified BIO3-BIO1 enzyme according to claim 26, wherein the BIO3-BIO1 enzyme is further modified to comprise a heterologous targeting peptide, preferably a heterologous mitochondrial targeting peptide.

28. An isolated polynucleotide encoding a modified BIO3-BIO1 enzyme according to claim 26.

29. An expression construct comprising an isolated polynucleotide according to claim 28.

30. A vector comprising the expression construct of claim 29.

31. A method of producing a plant or part thereof according to claim 16, the method comprising: modifying the plant or part thereof to comprise the BIO3-BIO1 enzyme and/or BioA enzyme that provides increased resistance to a compound which inhibits the biotin synthesis pathway relative to an unmodified reference plant.

32. A method according to claim 31, wherein modifying the plant or part thereof comprises transforming the plant or part thereof with a polynucleotide encoding the BIO3-BIO1 enzyme and/or BioA enzyme the expression of which provides increased resistance to a compound which inhibits the biotin synthesis pathway relative to an unmodified reference plant.

33. A method of producing a plant or part thereof according to claim 31, wherein the compound is a herbicide, preferably wherein the compound is a herbicide selected from: a herbicidal cinnoline compound such as ethyl 5-acetyl-4-oxo-1-(2,2,3,3-tetrafluoro-1,4-benzodioxin-6-yl)cinnoline-3-carboxylate (Compound A); a herbicidal compound such as [5-carboxy-1-(1-hydroxyethyl)pentyl]ammonium;chloride (compound B); a herbicidal pyridone compound such as 2-(3,4-dichlorophenyl)-5-(2,4-difluorophenyl)-1-ethyl-6-methyl-4-oxo-pyridine-3-carboxylic acid (Compound C) or 5-[2-chloro-5-(trifluoromethoxy)phenyl]-2-(3,4-dichlorophenyl)-1-ethyl-6-methyl-4-oxo-pyridine-3-carboxylic acid (Compound D); a herbicidal compound such as [(1R)-2-(6-carboxypyridin-1-ium-2-yl)oxy-1-methyl-ethyl]ammonium;dichloride (Compound E); a herbicidal pyrrolidine-2-one such as 2-[1-[(2,3-difluorophenyl)methyl]-5-oxo-pyrrolidin-2-yl]-N-(2-methyl-1,2,4-triazol-3-yl)acetamide (Compound F), 2-[1-[(2,3-difluorophenyl)methyl]-5-oxo-pyrrolidin-2-yl]acetic acid (Compound G), 2-[5-oxo-1-[(2,3,5-trifluorophenyl)methyl]pyrrolidin-2-yl]acetic acid (Compound H), 2-(4-fluorophenoxy)ethyl 2-[1-[(2,3-difluorophenyl)methyl]-5-oxo-pyrrolidin-2-yl]acetate (Compound I), 2-methoxyethyl 2-[1-[(2,3-difluorophenyl)methyl]-5-oxo-pyrrolidin-2-yl]acetate (Compound J), 2-[5-oxo-1-[(2,3,4-trifluorophenyl)methyl]pyrrolidin-2-yl]acetic acid (Compound K); a N-benzyl azole herbicidal compound such as 2-(3,4-dichlorophenyl)-1-ethyl-4-oxo-6-[[3-(trifluoromethyl)pyrazol-1-yl]methyl]pyridine-3-carboxylic acid (Compound L); and a herbicidal quinolone compound such as 2-(3,4-dichlorophenyl)-1-ethyl-6-fluoro-4-oxo-quinoline-3-carboxylic acid (Compound M).
